(19) United States
(12) Patent Application Publication
(10) Pub. No.: US 2024/0043371 A1
(43) Pub. Date: Feb. 8, 2024

(54) NOVEL SPIROFLUORENE COMPOUND AND ORGANIC LIGHTEMITTING DEVICE INCLUDING SAME

(71) Applicant: CENTUM MATERIALS INC., Gyeonggi-do (KR)

(72) Inventors: Sung-Jin EUM, Gyeonggi-do (KR); Sung Min KIM, Seoul (KR)

(21) Appl. No.: 18/257,497

(22) PCT Filed: Dec. 9, 2021
(86) PCT No.: PCT/KR2021/018588

§ 371 Date: Jun. 14, 2023

(30) Foreign Application Priority Data
Dec. 16, 2020 (KR) 10-2020-0176591

(51) Int. Cl. C07C 211/54; C07D 307/91; C07C 211/61; C07D 209/82; C07D 405/12; C07D 403/12; H10K 85/60; H10K 50/11

(52) U.S. Cl. CPC C07C 211/54; C07D 307/91; C07C 211/61; C07D 209/82; C07D 405/12; C07D 403/12; H10K 85/633; H10K 85/636; H10K 50/11

(57) ABSTRACT

The present invention relates to a novel spirofluorene compound and an organic light-emitting device including same. The spirofluorene compound according to the present invention is a polycyclic compound to which at least one amino substituent is introduced and which has a structure in which indene, indole, benzofuran, benzothiophene, or benzosilole is fused to a 9,9′-spirobifluorene moiety.

### TECHNICAL FIELD

The present invention relates to a novel spirofluorene compound and an organic light-emitting device including the same.

### BACKGROUND ART

In general, an organic light-emitting phenomenon refers to a phenomenon that converts electrical energy into light energy using an organic material. An organic electronic device using the organic light-emitting phenomenon has a structure which generally includes an anode, a cathode, and an organic material layer provided between the anode and the cathode. Here, most organic material layers have a multi-layer structure formed of different materials in order to increase efficiency and stability of the organic electronic device, and for example, the organic material layer may include a hole injection layer, a hole transport layer, an emissive layer, an electron transport layer, an electron injection layer, and the like.

A material used in the organic material layer in the organic electronic device may be classified into a light-emitting material and a charge transport material, for example, a hole injection material, a hole transport material, an electron transport material, an electron injection material, and the like. In addition, the light-emitting material may be classified into a high molecular weight material and a low molecular weight material, and may be classified into a fluorescent material derived from a singlet excited state of electrons and a phosphorescent material derived from a triplet excited state of electrons according to a light-emitting mechanism. In addition, the light-emitting material may be classified into blue, green, and red light-emitting materials and yellow and orange light-emitting materials required to realize better natural colors according to a light-emitting color.

In particular, many studies on an organic material inserted into a hole transport layer or a buffer layer for excellent life characteristics of the organic electronic device have been conducted. To this end, there is a demand for a material of the hole injection layer having high uniformity and low crystallinity when a thin film is formed after deposition while imparting high hole transport characteristics to the organic material layer from the anode.

There is also a demand for development of a material of the hole injection layer that delays penetration and diffusion of a metal oxide into the organic material layer from an anode electrode (ITO), which is one of the causes of a shortened life of the organic electronic device, and has stable properties, that is, a high glass transition temperature, even against Joule heating generated during driving of the device. In addition, it has been reported that a low glass transition temperature of a material of the hole transport layer greatly affects the life of the device according to the property that the uniformity of the surface of the thin film collapses during driving of the device. In addition, in the formation of an organic light-emitting diode (OLED) device, a deposition method is mainly used, and a material having strong heat resistance that may withstand such a deposition method for a long time is required.

Meanwhile, in a case where only one material is used as a light-emitting material, there are problems such as a shift of a maximum emission wavelength to a longer wavelength due to intermolecular interactions and a reduction in efficiency of the device due to a decrease in color purity or a reduction in emission efficiency. Therefore, a host/dopant system may be used as the light-emitting material in order to increase the color purity and increase the emission efficiency through energy transfer. This is based on the principle that when a small amount of dopant having a smaller energy gap than a host forming an emissive layer is mixed in the emissive layer, excitons generated in the emissive layer are transported to the dopant, and thus, light is emitted with high efficiency. In this case, the wavelength of the host shifts to the wavelength band of the dopant, and light having a desired wavelength may thus be obtained according to the type of the dopant.

In order to fully exhibit excellent characteristics of the organic electronic device, materials constituting the organic material layer in the device, such as a hole injection material, a hole transport material, a light-emitting material, an electron transport material, and an electron injection material, should be stable and efficient. However, a stable and efficient material of an organic material layer for an organic electronic device has not yet been sufficiently developed, and thus, development of a novel material has been continuously required.

### DISCLOSURE

### Technical Problem

An object of the present invention is to provide a spirofluorene compound having a novel structure that may be used as a material of an organic material layer of an organic light-emitting device.

Another object of the present invention is to provide an organic light-emitting device including the spirofluorene compound as a material of an organic material layer.

### Technical Solution

The present invention relates to a novel spirofluorene compound and an organic light-emitting device including the same. The spirofluorene compound according to the present invention has a structure in which a polycyclic ring is fused with a 9,9′-spirobifluorene moiety, and may be used as a material of an organic material layer of an organic light-emitting device.

In one general aspect, there is provided a spirofluorene compound represented by the following Chemical Formula 1:

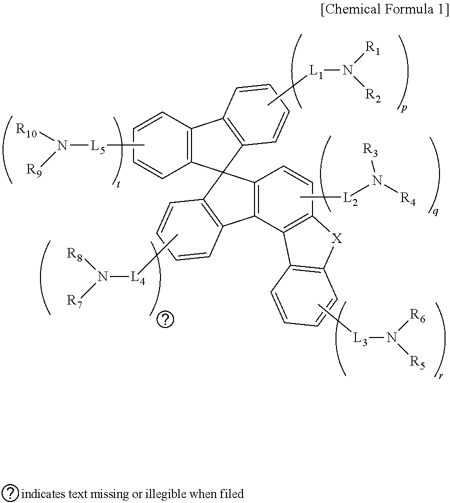

in Chemical Formula 1,
X is CR^{a}R^{b}, NR^{c}, O, S, or SiR^{d}R^{e};
R^{a}, R^{b}, R^{c}, R^{d}, and R^{e} are each independently hydrogen, deuterium, C1-C60 alkyl, C3-C60 cycloalkyl, C6-C60 aryl, or C3-C60 heteroaryl;
L₁ to L₅ are each independently a single bond, C6-C60 arylene, or C3-C60 heteroarylene;
R₁ to R₁₀ are each independently C1-C60 alkyl, C2-C60 alkenyl, C2-C60 alkynyl, C3-C60 cycloalkyl, C2-C60 heterocycloalkyl, C6-C60 aryl, C3-C60 heteroaryl, or -L₁₁-R₁₁;
L₁₁ is C6-C60 arylene or C3-C60 heteroarylene;
R₁₁ is C6-C60 aryl, C3-C60 heteroaryl, or —NR₁₂R₁₃;
R₁₂ and R₁₃ are each independently hydrogen, C1-C60 alkyl, C6-C60 aryl, or C3-C60 heteroaryl;
the arylene and heteroarylene of L₁ to L₅, the alkyl, alkenyl, alkynyl, cycloalkyl, heterocycloalkyl, aryl, and heteroaryl of R₁ to R₁₀, the arylene and heteroarylene of L₁₁, the aryl and heteroaryl of R₁₁, and the alkyl, aryl, and heteroaryl of R₁₂ and R₁₃ may be further substituted by one or more selected from the group consisting of C1-C60 alkyl, halo C1-C60 alkyl, deuterium, halogen, cyano, C3-C60 cycloalkyl, C1-C60 alkoxy, C6-C60 aryl, C6-C60 aryloxy, C6-C60 aryl C1-C60 alkyl, C1-C60 alkyl C6-C60 aryl, C3-C60 heteroaryl, —NR′R″, nitro, and hydroxy;
R′ and R″ are each independently hydrogen, C1-C60 alkyl, C6-C60 aryl, or C3-C60 heteroaryl;
p, r, s, and t are each independently an integer of 0 to 4, and q is an integer of 0 to 2, but p, q, r, s, and t are not simultaneously 0; and
the heteroarylene and heteroaryl contain one or more heteroatoms selected from N, O, S, and Se.

In another general aspect, an organic light-emitting device includes: an anode; a cathode; and one or more organic material layers provided between the anode and the cathode, wherein one or more layers of the organic material layers include the spirofluorene compound of Chemical Formula 1.

### Advantageous Effects

The spirofluorene compound according to the present invention is a polycyclic compound into which at least one amino substituent is introduced, the polycyclic compound having a skeleton in which a substituted or unsubstituted polycyclic ring, specifically, indene, indole, benzofuran, benzothiophene, or benzosilole, is fused with a 9,9′-spirobifluorene moiety, and may be used as a material of an organic material layer of an organic light-emitting device. The spirofluorene compound may act as a light-emitting material, a hole injection material, a hole transport material, an electron transport material, an electron injection material, or the like in an organic light-emitting device.

The spirofluorene compound according to the present invention may be employed as a material of an organic material layer such as a light-emitting material, a hole injection material, a hole transport material, an electron transport material, or an electron injection material due to its structural specificity, and therefore, an organic light-emitting device employing the same has high hole mobility, and thus, may have both high efficiency and a low drive voltage and may also have a significantly improved life.

That is, since the spirofluorene compound according to the present invention has excellent electron mobility due to its structural specificity, current characteristics of the device are improved to intensify the drive voltage, which induces an increase in power efficiency, such that it is possible to manufacture an organic light-emitting device having improved power consumption.

### BEST MODE

Hereinafter, the present invention will be described. However, technical terms and scientific terms used herein have the general meanings understood by those skilled in the art to which the present invention pertains unless otherwise defined, and a description for the known function and configuration unnecessarily obscuring the gist of the present invention will be omitted in the following description.

It should be understood that in the present specification, unless otherwise required in the context, the terms “comprise” and “comprising” include a suggested step or constituent element, or a group of steps or constituent elements, but imply that any other step or constituent element or any group of steps or constituent groups is not excluded.

In the present specification, the terms “substituent”, “radical”, “group”, “moiety”, and “fragment” may be used interchangeably.

In the present specification, the term “C_{A}-C_{B}” means that “the number of carbon atoms is A or more and B or less”.

In the present specification, the terms “alkyl”, “alkoxy”, and other substituents containing “alkyl” moiety include all linear or branched forms.

In the present specification, “alkyl” includes linear or branched alkyl having 1 to 60 carbon atoms, and may be further substituted by other substituents. The number of carbon atoms of alkyl may be 1 to 60, specifically, 1 to 30, and more specifically, 1 to 20, and may be preferably 1 to 10.

In the present specification, “alkenyl” includes linear or branched alkenyl having 2 to 60 carbon atoms, and may be further substituted by other substituents. The number of carbon atoms of alkenyl may be 2 to 60, specifically, 2 to 30, and more specifically, 2 to 20, and may be preferably 2 to 10.

In the present specification, “alkynyl” includes linear or branched alkynyl having 2 to 60 carbon atoms, and may be further substituted by other substituents. The number of carbon atoms of alkynyl may be 2 to 60, specifically, 2 to 30, and more specifically, 2 to 20, and may be preferably 2 to 10.

In the present specification, “cycloalkyl” includes monocyclic or polycyclic cycloalkyl having 3 to 60 carbon atoms, and may be further substituted by other substituents. Here, the term “polycyclic” means a group in which cycloalkyl is directly connected to or fused with other ring groups. Here, the term “other ring groups” may be cycloalkyl, and may also be other types of ring groups, for example, heterocycloalkyl, aryl, heterocycle, and the like. The number of carbon atoms of cycloalkyl may be 3 to 60, specifically, 3 to 30, and more specifically, 5 to 20.

In the present specification, “heterocycloalkyl” includes at least one selected from N, O, S, and Se as a heteroatom, includes monocyclic or polycyclic heterocycloalkyl having 2 to 60 carbon atoms, and may be further substituted by other substituents. Here, the term “polycyclic” means a group in which heterocycloalkyl is directly connected to or fused with other ring groups. Here, the term “other ring groups” may be heterocycloalkyl, and may also be other types of ring groups, for example, cycloalkyl, aryl, heterocycle, and the like. The number of carbon atoms of heterocycloalkyl may be 2 to 60, specifically, 2 to 30, and more specifically, 3 to 20.

In the present specification, “aryl” is an organic radical derived from an aromatic hydrocarbon by removing one hydrogen atom, includes monocyclic or polycyclic aryl having 6 to 60 carbon atoms, and may be further substituted by other substituents. Here, the term “polycyclic” means a group in which aryl is directly connected to or fused with other ring groups. Here, the term “other ring groups” may be aryl, and may also be other types of ring groups, for example, cycloalkyl, heterocycloalkyl, heterocycle, and the like. The number of carbon atoms of aryl may be 6 to 60, specifically, 6 to 30, and more specifically, 6 to 25. Specific examples of aryl include, but are not limited to, phenyl, biphenyl, triphenyl, naphthyl, anthryl, chrysenyl, phenanthrenyl, perylenyl, fluoranthenyl, triphenylenyl, phenalenyl, pyrenyl, tetracenyl, pentacenyl, fluorenyl, indenyl, acenaphthylenyl, and fused rings thereof.

In the present specification, the term “arylene” means a divalent organic radical derived by removing one hydrogen atom from the above aryl, and follows the above definition of aryl.

In the present specification, a “heterocyclic group” includes at least one selected from N, O, S, and Se as a heteroatom, includes a monocyclic or polycyclic heterocyclic group having 2 to 60 carbon atoms, and may be further substituted by other substituents. Heteroaryl is included in the scope of the heterocyclic group, and is a heteroaromatic ring group. Here, the term “polycyclic” means a group in which a heterocyclic group is directly connected to or fused with other ring groups. Here, the term “other ring groups” may be a heterocyclic group, and may also be other types of ring groups, for example, cycloalkyl, heterocycloalkyl, aryl, and the like. The number of carbon atoms of the heterocyclic group may be 2 to 60, specifically, 2 to 30, and more specifically, 3 to 25. Specific examples of the heterocyclic group include, but are not limited to, pyridyl, pyrrolyl, pyrimidyl, pyridazinyl, furanyl, thienyl, imidazolyl, pyrazolyl, oxazolyl, isoxazolyl, thiazolyl, isothiazolyl, triazolyl, furazanyl, oxadiazolyl, thiadiazolyl, dithiazolyl, tetrazolyl, pyranyl, thiopyranyl, diazinyl, oxazinyl, thiazinyl, dioxynyl, triazinyl, tetrazinyl, quinolyl, isoquinolyl, quinazolinyl, isoquinazolinyl, naphthyridyl, acridinyl, phenanthridinyl, imidazopyridinyl, diazanaphthalenyl, triazaindene, indolyl, indolizinyl, benzothiazolyl, benzoxazolyl, benzoimidazolyl, a benzothiophene group, a benzofuran group, a dibenzothiophene group, a dibenzofuran group, carbazolyl, benzocarbazolyl, phenazinyl, and fused rings thereof.

In the present specification, the term “heteroaryl” means an aryl group containing at least one heteroatom selected from N, O, S, and Se as an aromatic ring skeleton atom and carbon as the remaining aromatic ring skeleton atoms, is 5- or 6-membered monocyclic heteroaryl or polycyclic heteroaryl which is fused with one or more benzene rings, and may be partially saturated. In addition, heteroaryl in the present invention includes a form in which one or more heteroaryls are linked to one another by a single bond. Specific examples thereof include, but are not limited to, monocyclic heteroaryl such as furyl, thiophenyl, pyrrolyl, imidazolyl, pyrazolyl, thiazolyl, isothiazolyl, isoxazolyl, oxazolyl, triazinyl, pyridyl, pyrazinyl, pyrimidinyl, or pyridazinyl; and polycyclic heteroaryl such as benzofuranyl, benzothiophenyl, isobenzofuranyl, benzoimidazolyl, benzothiazolyl, benzoisothiazolyl, benzoisoxazolyl, benzoxazolyl, isoindolyl, indolyl, indazolyl, quinolyl, isoquinolyl, dibenzofuranyl, dibenzothiophenyl, carbazolyl, or benzocarbazolyl.

In the present specification, the term “heteroarylene” means a divalent organic radical derived by removing one hydrogen atom from the above heteroaryl, and follows the above definition of heteroaryl.

The present invention relates to a novel spirofluorene compound and an organic light-emitting device including the same, and more particularly, provides a spirofluorene compound having a structure in which a polycyclic ring is fused with a 9,9′-spirobifluorene moiety, and an organic light-emitting device including the same.

A spirofluorene compound according to the present invention may be represented by the following Chemical Formula 1:

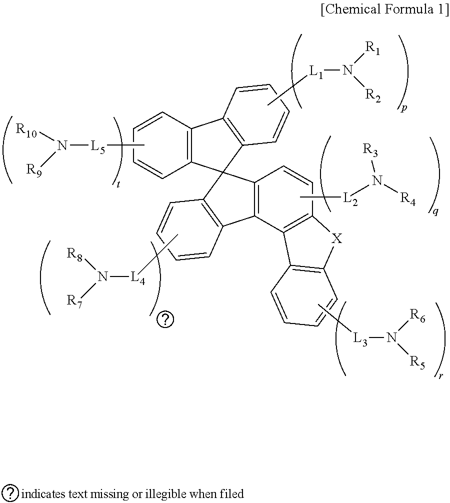

in Chemical Formula 1,
X is CR^{a}R^{b}, NR^{c}, O, S, or SiR^{d}R^{e};
R^{a}, R^{b}, R^{c}, R^{d}, and R^{e} are each independently hydrogen, deuterium, C1-C60 alkyl, C3-C60 cycloalkyl, C6-C60 aryl, or C3-C60 heteroaryl;
L₁ to L₅ are each independently a single bond, C6-C60 arylene, or C3-C60 heteroarylene;
R₁ to R₁₀ are each independently C1-C60 alkyl, C2-C60 alkenyl, C2-C60 alkynyl, C3-C60 cycloalkyl, C2-C60 heterocycloalkyl, C6-C60 aryl, C3-C60 heteroaryl, or -L₁₁-R₁₁;
L₁₁ is C6-C60 arylene or C3-C60 heteroarylene;
R₁₁ is C6-C60 aryl, C3-C60 heteroaryl, or —NR₁₂R₁₃;
R₁₂ and R₁₃ are each independently hydrogen, C1-C60 alkyl, C6-C60 aryl, or C3-C60 heteroaryl;
the arylene and heteroarylene of L₁ to L₅, the alkyl, alkenyl, alkynyl, cycloalkyl, heterocycloalkyl, aryl, and heteroaryl of R₁ to R₁₀, the arylene and heteroarylene of L₁₁, the aryl and heteroaryl of R₁₁, and the alkyl, aryl, and heteroaryl of R₁₂ and R₁₃ may be further substituted by one or more selected from the group consisting of C1-C60 alkyl, halo C1-C60 alkyl, deuterium, halogen, cyano, C3-C60 cycloalkyl, C1-C60 alkoxy, C6-C60 aryl, C6-C60 aryloxy, C6-C60 aryl C1-C60 alkyl, C1-C60 alkyl C6-C60 aryl, C3-C60 heteroaryl, —NR′R″, nitro, and hydroxy;
R′ and R″ are each independently hydrogen, C1-C60 alkyl, C6-C60 aryl, or C3-C60 heteroaryl;
p, r, s, and t are each independently an integer of 0 to 4, and q is an integer of 0 to 2, but p, q, r, s, and t are not simultaneously 0; and
the heteroarylene and heteroaryl contain one or more heteroatoms selected from N, O, S, and Se.

Specifically, the spirofluorene compound of Chemical Formula 1 is a polycyclic compound into which at least one amino substituent is introduced, the polycyclic compound having a structure in which a polycyclic ring, specifically, indene, indole, benzofuran, benzothiophene, or benzosilole, is fused with a 9,9′-spirobifluorene moiety, and may be used as a material of an organic material layer of an organic light-emitting device.

When the spirofluorene compound of Chemical Formula 1 is employed as a material of an organic material layer of an organic light-emitting device, in particular, a hole transport material of an organic light-emitting device, due to the structural characteristics thereof, it is possible to reduce a drive voltage, improve emission efficiency and color purity, and exhibit significantly improved life characteristics.

In Chemical Formula 1 according to an embodiment, X may be CR^{a}R^{b}, NR^{c}, O, S, or SiR^{d}R^{e}; R^{a}, R^{b}, R^{c}, R^{d}, and R^{e} may be each independently hydrogen, deuterium, C1-C60 alkyl, or C6-C60 aryl; L₁ to L₅ may be each independently a single bond, C6-C60 arylene, or C3-C60 heteroarylene, the arylene and heteroarylene of L₁ to L₅ may be further substituted by one or more selected from the group consisting of C1-C60 alkyl, C6-C30 aryl, and —NR′R″; R′ and R″ may be each independently C6-C60 aryl or C3-C60 heteroaryl; R₁ to R₁₀ may be each independently C6-C60 aryl, C3-C60 heteroaryl, or -L₁₁-R₁₁; L₁₁ may be C6-C60 arylene or C3-C60 heteroarylene; R₁₁ may be C6-C60 aryl, C3-C60 heteroaryl, or —NR₁₂R₁₃; R₁₂ and R₁₃ may be each independently C6-C60 aryl or C3-C60 heteroaryl; the aryl and heteroaryl of R₁ to R₁₀, the arylene and heteroarylene of L₁₁, the aryl and heteroaryl of R₁₁, and the aryl and heteroaryl of R₁₂ and R₁₃ may be further substituted by one or more selected from the group consisting of C1-C60 alkyl, deuterium, C6-C60 aryl, C6-C60 aryl C1-C60 alkyl, C1-C60 alkyl C6-C60 aryl, and C3-C60 heteroaryl; and p, q, r, s, and t may be each independently an integer of 0 to 2 and may satisfy 1≤p+q+r+s+t≤10.

In order to implement further improved device characteristics, in the spirofluorene compound, p+q+r+s+t may be an integer of 1 or 2.

In an embodiment, the spirofluorene compound may be represented by any one of the following Chemical Formulas 2 to 6:

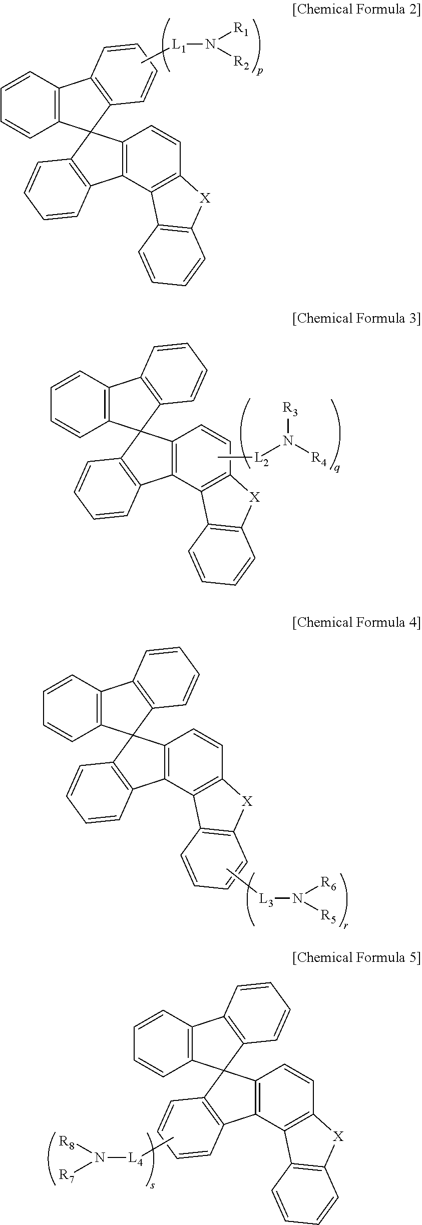

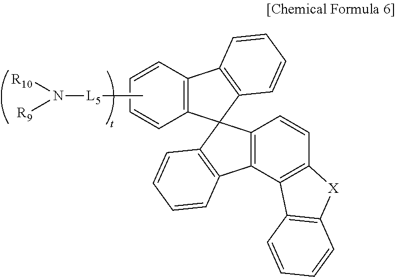

in Chemical Formulas 2 to 6,
X is CR^{a}R^{b}, NR^{c}, O, S, or SiR^{d}R^{e};
R^{a}, R^{b}, R^{c}, R^{d}, and R^{e} are each independently C1-C30 alkyl or C6-C30 aryl;
L₁ to L₅ are each independently a single bond, C6-C30 arylene, or C3-C30 heteroarylene, and the arylene and heteroarylene of L₁ to L₅ may be further substituted by one or more selected from the group consisting of C1-C30 alkyl, C6-C30 aryl, and —NR′R″;
R′ and R″ are each independently C6-C30 aryl or C3-C30 heteroaryl;
R₁ to R₁₀ are each independently C6-C30 aryl, C3-C30 heteroaryl, or -L₁₁-R₁₁;
L₁₁ is C6-C30 arylene or C3-C30 heteroarylene;
R₁₁ is C6-C30 aryl, C3-C30 heteroaryl, or —NR₁₂R₁₃;
R₁₂ and R₁₃ are each independently C6-C30 aryl or C3-C30 heteroaryl;
the aryl and heteroaryl of R₁ to R₁₀, the arylene and heteroarylene of L₁₁, the aryl and heteroaryl of R₁₁, and the aryl and heteroaryl of R₁₂ and R₁₃ may be further substituted by one or more selected from the group consisting of C1-C30 alkyl, C6-C30 aryl, C6-C30 aryl C1-C30 alkyl, C1-C30 alkyl C6-C30 aryl, and C3-C30 heteroaryl; and
p, q, r, s, and t are each independently an integer of 1 or 2.

In an embodiment, the spirofluorene compound may be represented by any one of the following Chemical Formulas 7 to 13:

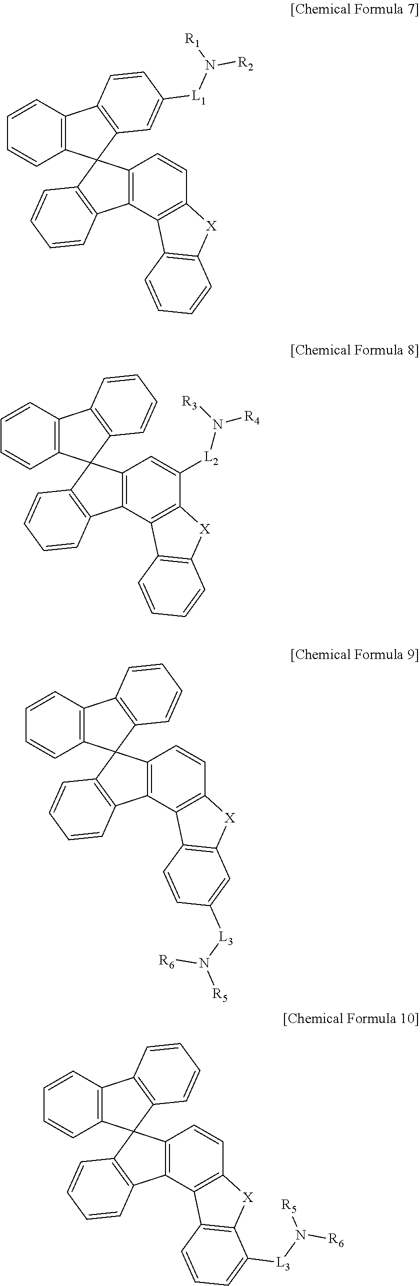

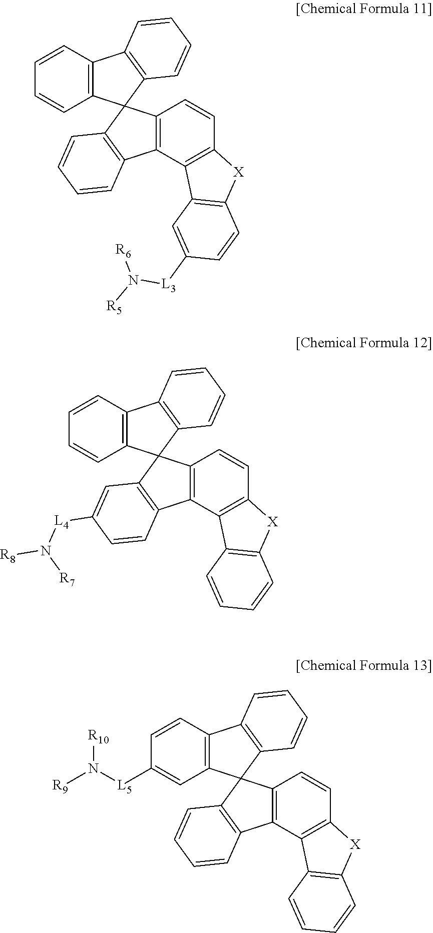

in Chemical Formulas 7 to 13,
X is CR^{a}R^{b}, NR^{c}, or O;
R^{a} and R^{b} are each independently C1-C20 alkyl;
R^{c} is C6-C20 aryl;
L₁ to L₅ are each independently a single bond, C6-C20 arylene, or C3-C20 heteroarylene, and the arylene and heteroarylene of L₁ to L₅ may be further substituted by one or more selected from the group consisting of C1-C20 alkyl, C6-C20 aryl, and —NR′R″;
R′ and R″ are each independently C6-C20 aryl or C3-C20 heteroaryl;
R₁ to R₁₀ are each independently C6-C20 aryl, C3-C20 heteroaryl, or -L₁₁-R₁₁;
L₁₁ is C6-C20 arylene or C3-C20 heteroarylene;
R₁₁ is C6-C20 aryl, C3-C20 heteroaryl, or —NR₁₂R₁₃;
R₁₂ and R₁₃ are each independently C6-C20 aryl or C3-C20 heteroaryl; and
the aryl and heteroaryl of R₁ to R₁₀, the arylene and heteroarylene of L₁₁, the aryl and heteroaryl of R₁₁, and the aryl and heteroaryl of R₁₂ and R₁₃ may be further substituted by one or more selected from the group consisting of C1-C20 alkyl, C6-C20 aryl, C6-C20 aryl C1-C20 alkyl, C1-C20 alkyl C6-C20 aryl, and C3-C20 heteroaryl.

In an embodiment, L₁ to L₅ may be each independently a single bond or selected from the following structures, but are not limited to:

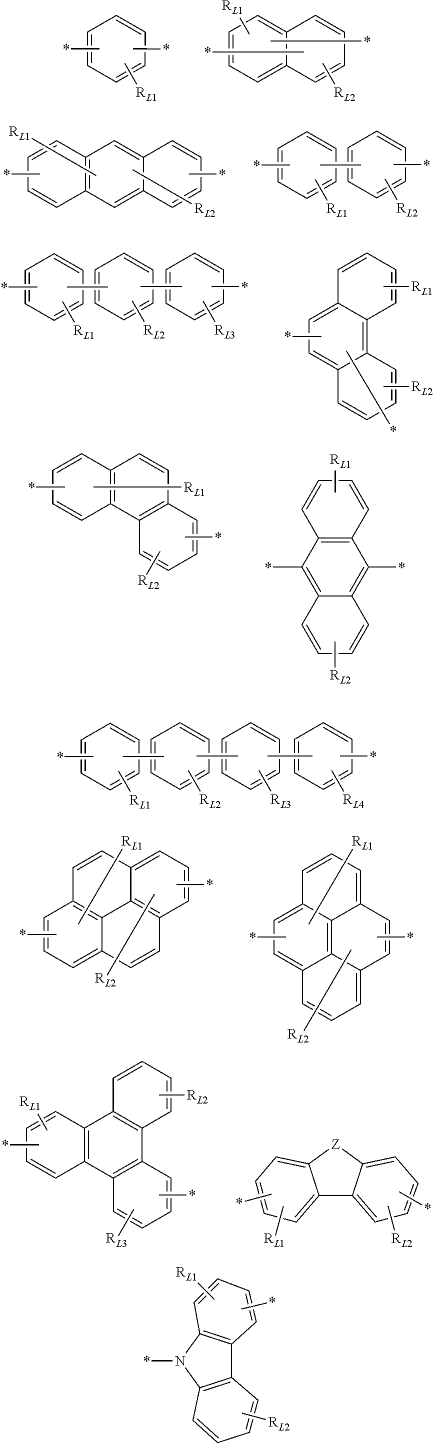

wherein
R_{L1}, R_{L2}, R_{L3}, and R_{L4} are each independently hydrogen, C6-C20 aryl, or NR′R″;
R′ and R″ are each independently C6-C20 aryl or C3-C20 heteroaryl;
Z is CR_{Z1}R_{Z2}, NR_{Z3}, O, or S;
R_{Z1} and R_{Z2} are each independently C1-C20 alkyl or C6-C20 aryl; and
R_{Z3} is C6-C20 aryl.

In an embodiment, R₁ to R₁₀ may be each independently selected from the following structures, but are not limited to:

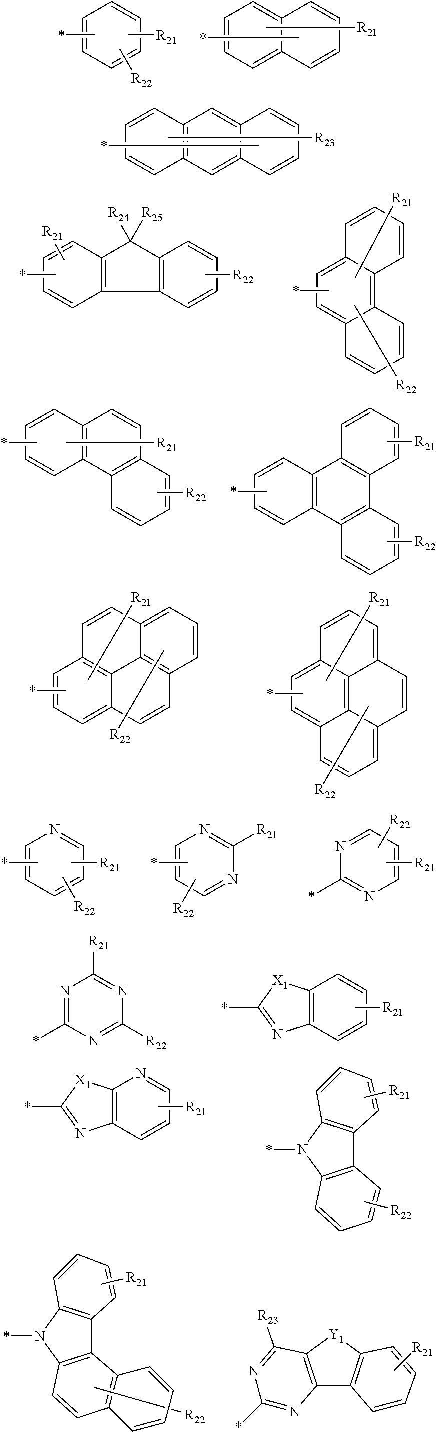

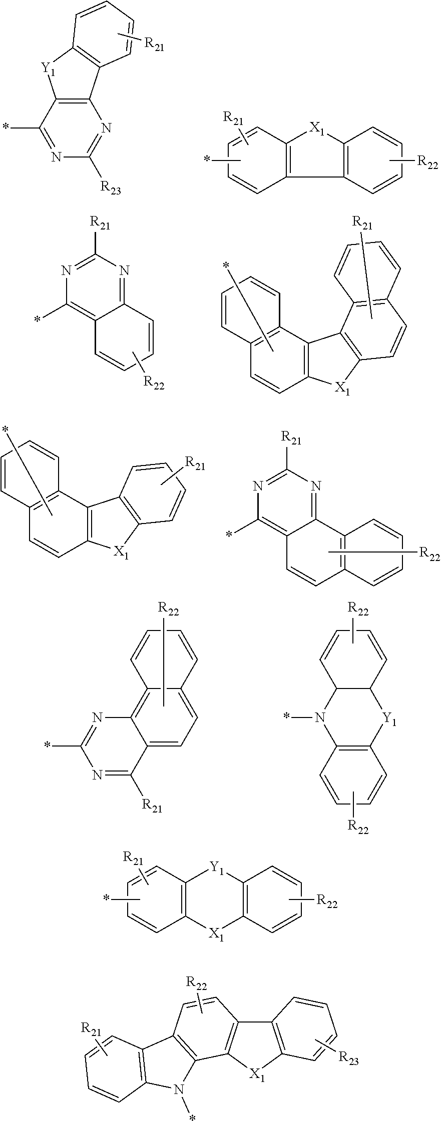

wherein
X₁ is NR₃₁, O, or S;
Y₁ is CR₃₂R₃₃, O, or S;
R₃₁ is C6-C20 aryl or C3-C20 heteroaryl;
R₃₂ and R₃₃ are each independently C1-C20 alkyl, C6-C20 aryl, or C3-C20 heteroaryl;
R₂₁, R₂₂, and R₂₃ are each independently hydrogen, C6-C20 aryl, C3-C20 heteroaryl, or —NR₁₂R₁₃;
R₁₂ and R₁₃ are each independently C6-C20 aryl or C3-C20 heteroaryl;
R₂₄ and R₂₅ are each independently C1-C20 alkyl or C6-C20 aryl; and
the aryl and heteroaryl of R₂₁, R₂₂, and R₂₃ may be further substituted by one or more selected from the group consisting of C1-C20 alkyl and C6-C20 aryl.

In an embodiment, the spirofluorene compound may be selected from the following compounds, but is not limited thereto:

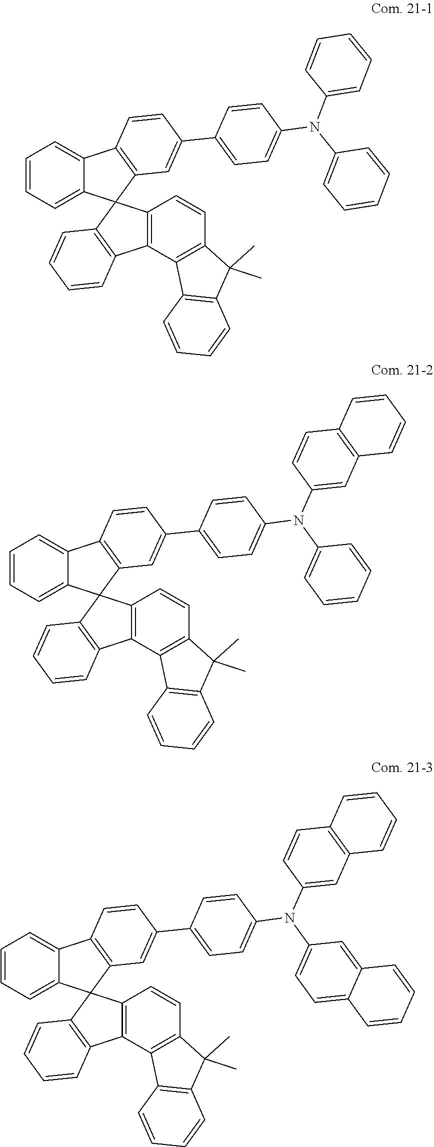 [SMILES: CC1(C)c2ccccc2-c2c1ccc1c2-c2ccccc2C12c1ccccc1-c1ccc(-c3ccc(N(c4ccccc4)c4ccccc4)cc3)cc12 ; CC1(C)c2ccccc2-c2c1ccc1c2-c2ccccc2C12c1ccccc1-c1ccc(-c3ccc(N(c4ccccc4)c4ccc5ccccc5c4)cc3)cc12 ; CC1(C)c2ccccc2-c2c1ccc1c2-c2ccccc2C12c1ccccc1-c1ccc(-c3ccc(N(c4ccc5ccccc5c4)c4ccc5ccccc5c4)cc3)cc12]

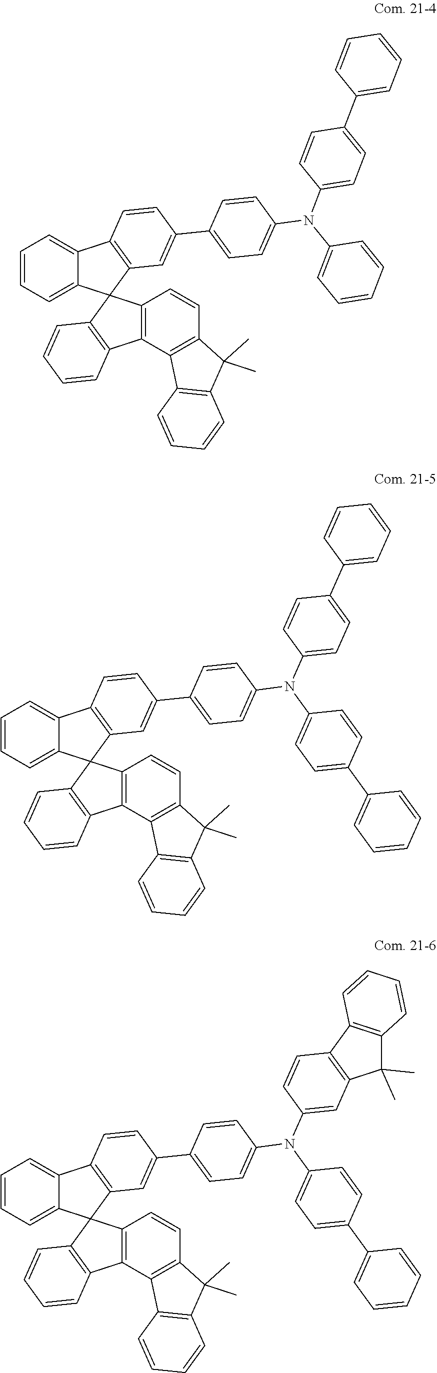 [SMILES: CC1(C)c2ccccc2-c2c1ccc1c2-c2ccccc2C12c1ccccc1-c1ccc(-c3ccc(N(c4ccccc4)c4ccc(-c5ccccc5)cc4)cc3)cc12 ; CC1(C)c2ccccc2-c2c1ccc1c2-c2ccccc2C12c1ccccc1-c1ccc(-c3ccc(N(c4ccc(-c5ccccc5)cc4)c4ccc(-c5ccccc5)cc4)cc3)cc12 ; CC1(C)c2ccccc2-c2ccc(N(c3ccc(-c4ccccc4)cc3)c3ccc(-c4ccc5c(c4)C4(c6ccccc6-5)c5ccccc5-c5c4ccc4c5-c5ccccc5C4(C)C)cc3)cc21]

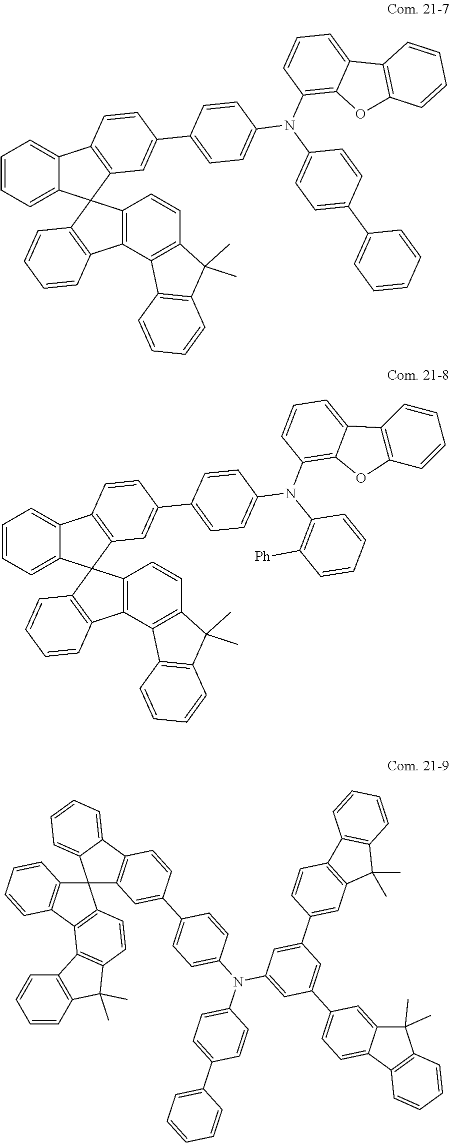 [SMILES: CC1(C)c2ccccc2-c2c1ccc1c2-c2ccccc2C12c1ccccc1-c1ccc(-c3ccc(N(c4ccc(-c5ccccc5)cc4)c4cccc5c4oc4ccccc45)cc3)cc12 ; CC1(C)c2ccccc2-c2c1ccc1c2-c2ccccc2C12c1ccccc1-c1ccc(-c3ccc(N(c4ccccc4-c4ccccc4)c4cccc5c4oc4ccccc45)cc3)cc12 ; CC1(C)c2ccccc2-c2ccc(-c3cc(-c4ccc5c(c4)C(C)(C)c4ccccc4-5)cc(N(c4ccc(-c5ccccc5)cc4)c4ccc(-c5ccc6c(c5)C5(c7ccccc7-6)c6ccccc6-c6c5ccc5c6-c6ccccc6C5(C)C)cc4)c3)cc21]

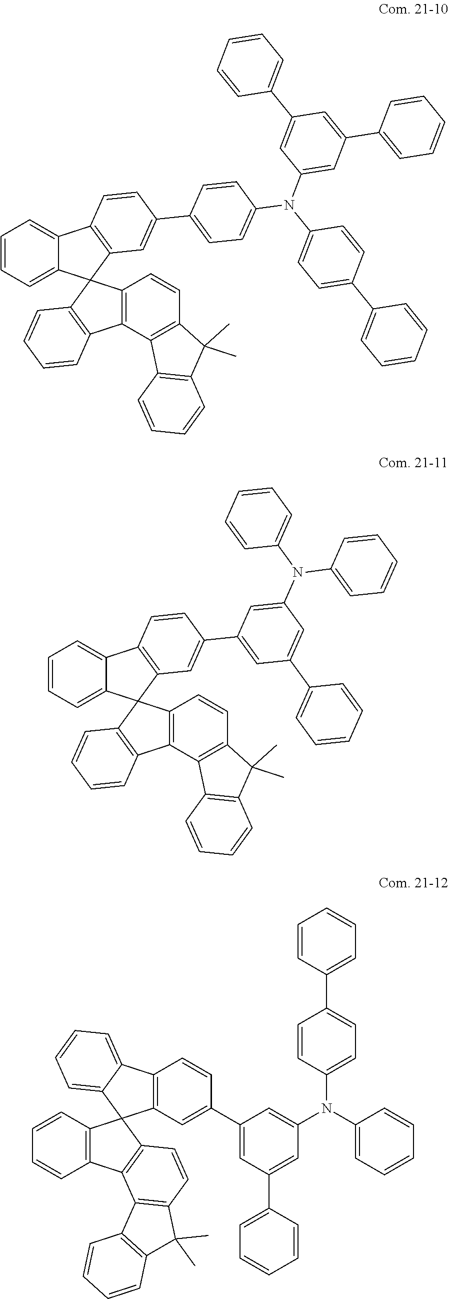 [SMILES: CC1(C)c2ccccc2-c2c1ccc1c2-c2ccccc2C12c1ccccc1-c1ccc(-c3ccc(N(c4ccc(-c5ccccc5)cc4)c4cc(-c5ccccc5)cc(-c5ccccc5)c4)cc3)cc12 ; CC1(C)c2ccccc2-c2c1ccc1c2-c2ccccc2C12c1ccccc1-c1ccc(-c3cc(-c4ccccc4)cc(N(c4ccccc4)c4ccccc4)c3)cc12 ; CC1(C)c2ccccc2-c2c1ccc1c2-c2ccccc2C12c1ccccc1-c1ccc(-c3cc(-c4ccccc4)cc(N(c4ccccc4)c4ccc(-c5ccccc5)cc4)c3)cc12]

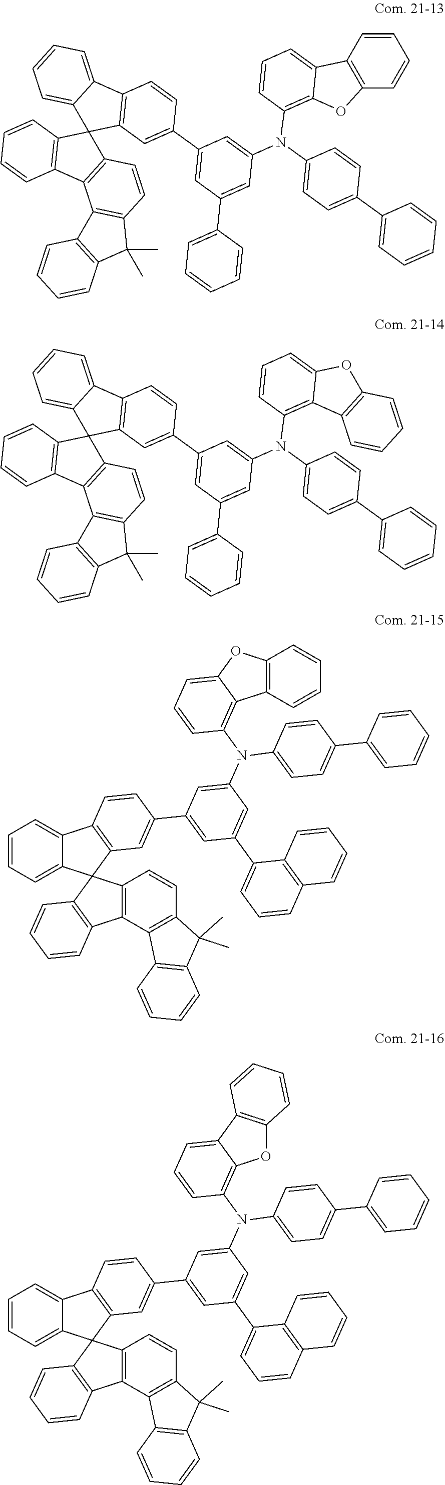

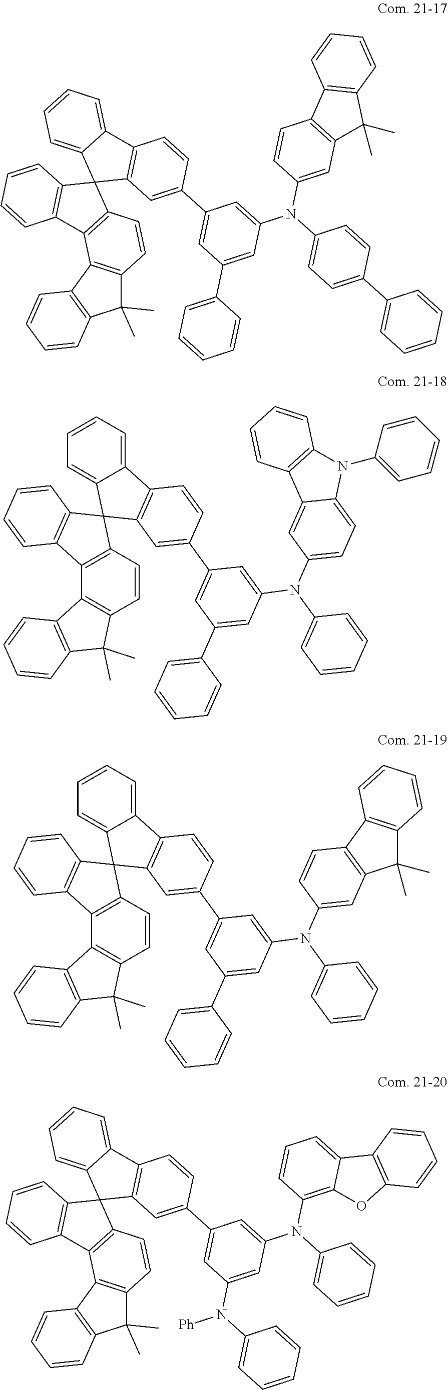 [SMILES: CC1(C)c2ccccc2-c2ccc(N(c3ccc(-c4ccccc4)cc3)c3cc(-c4ccccc4)cc(-c4ccc5c(c4)C4(c6ccccc6-5)c5ccccc5-c5c4ccc4c5-c5ccccc5C4(C)C)c3)cc21 ; CC1(C)c2ccccc2-c2c1ccc1c2-c2ccccc2C12c1ccccc1-c1ccc(-c3cc(-c4ccccc4)cc(N(c4ccccc4)c4ccc5c(c4)c4ccccc4n5-c4ccccc4)c3)cc12 ; CC1(C)c2ccccc2-c2ccc(N(c3ccccc3)c3cc(-c4ccccc4)cc(-c4ccc5c(c4)C4(c6ccccc6-5)c5ccccc5-c5c4ccc4c5-c5ccccc5C4(C)C)c3)cc21 ; CC1(C)c2ccccc2-c2c1ccc1c2-c2ccccc2C12c1ccccc1-c1ccc(-c3cc(N(c4ccccc4)c4ccccc4)cc(N(c4ccccc4)c4cccc5c4oc4ccccc45)c3)cc12]

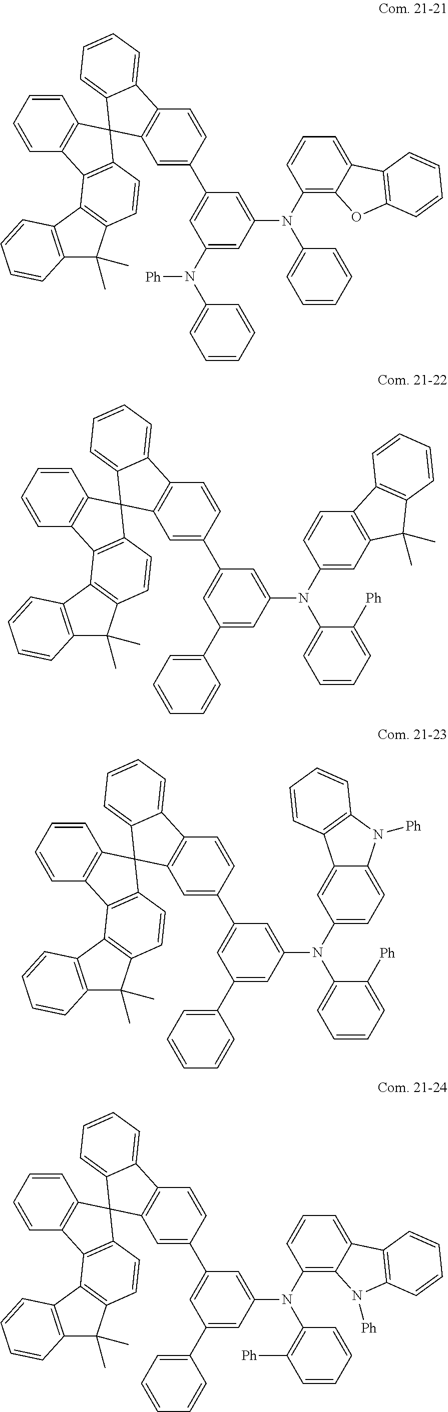

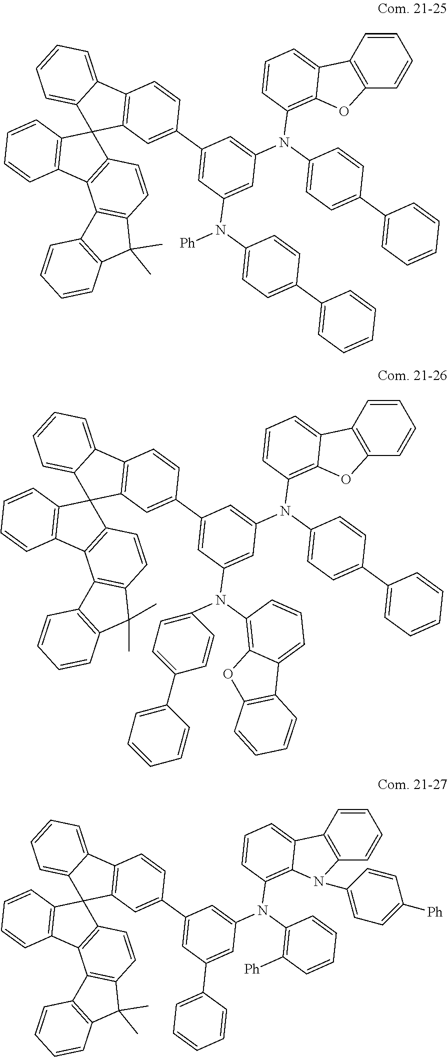 [SMILES: CC1(C)c2ccccc2-c2c1ccc1c2-c2ccccc2C12c1ccccc1-c1ccc(-c3cc(N(c4ccccc4)c4ccc(-c5ccccc5)cc4)cc(N(c4ccc(-c5ccccc5)cc4)c4cccc5c4oc4ccccc45)c3)cc12 ; CC1(C)c2ccccc2-c2c1ccc1c2-c2ccccc2C12c1ccccc1-c1ccc(-c3cc(N(c4ccc(-c5ccccc5)cc4)c4cccc5c4oc4ccccc45)cc(N(c4ccc(-c5ccccc5)cc4)c4cccc5c4oc4ccccc45)c3)cc12 ; CC1(C)c2ccccc2-c2c1ccc1c2-c2ccccc2C12c1ccccc1-c1ccc(-c3cc(-c4ccccc4)cc(N(c4ccccc4-c4ccccc4)c4cccc5c6ccccc6n(-c6ccc(-c7ccccc7)cc6)c45)c3)cc12]

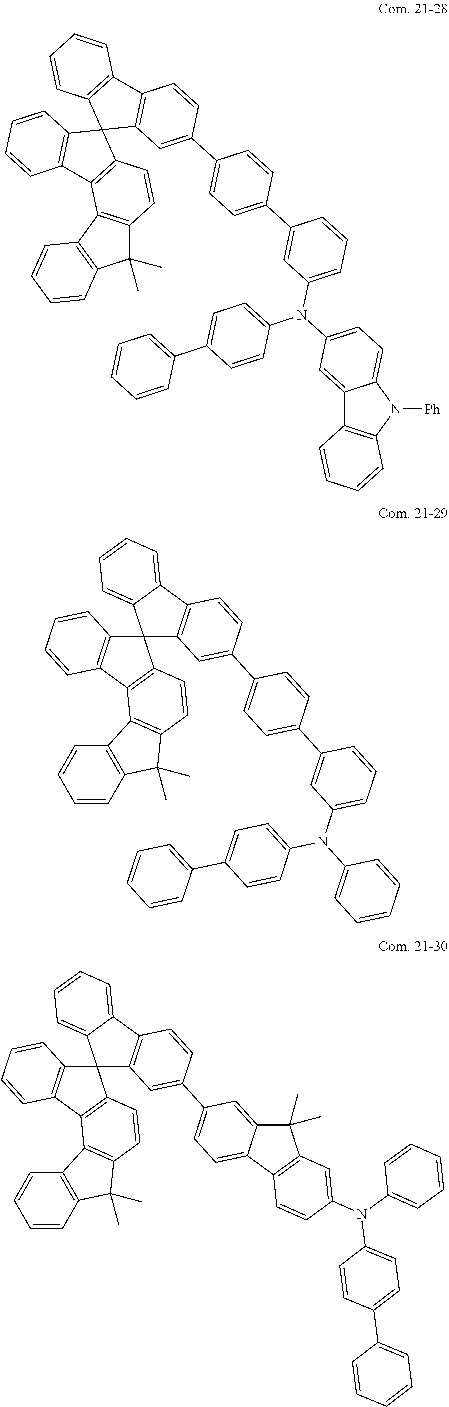 [SMILES: CC1(C)c2ccccc2-c2c1ccc1c2-c2ccccc2C12c1ccccc1-c1ccc(-c3ccc(-c4cccc(N(c5ccc(-c6ccccc6)cc5)c5ccc6c(c5)c5ccccc5n6-c5ccccc5)c4)cc3)cc12 ; CC1(C)c2ccccc2-c2c1ccc1c2-c2ccccc2C12c1ccccc1-c1ccc(-c3ccc(-c4cccc(N(c5ccccc5)c5ccc(-c6ccccc6)cc5)c4)cc3)cc12 ; CC1(C)c2cc(-c3ccc4c(c3)C3(c5ccccc5-4)c4ccccc4-c4c3ccc3c4-c4ccccc4C3(C)C)ccc2-c2ccc(N(c3ccccc3)c3ccc(-c4ccccc4)cc3)cc21]

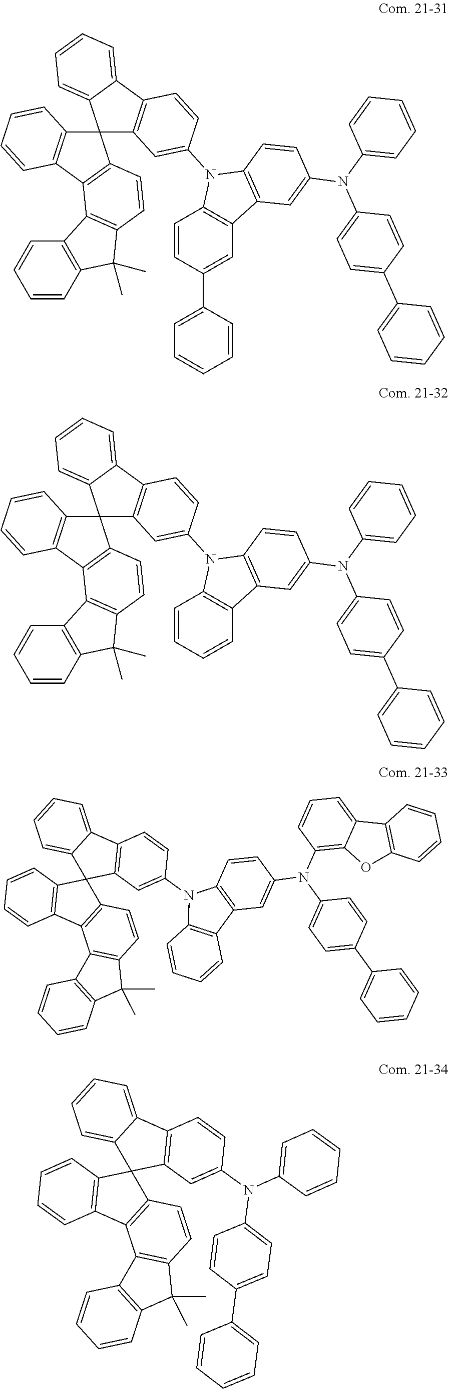 [SMILES: CC1(C)c2ccccc2-c2c1ccc1c2-c2ccccc2C12c1ccccc1-c1ccc(-n3c4ccc(-c5ccccc5)cc4c4cc(N(c5ccccc5)c5ccc(-c6ccccc6)cc5)ccc43)cc12 ; CC1(C)c2ccccc2-c2c1ccc1c2-c2ccccc2C12c1ccccc1-c1ccc(-n3c4ccccc4c4cc(N(c5ccccc5)c5ccc(-c6ccccc6)cc5)ccc43)cc12 ; CC1(C)c2ccccc2-c2c1ccc1c2-c2ccccc2C12c1ccccc1-c1ccc(-n3c4ccccc4c4cc(N(c5ccc(-c6ccccc6)cc5)c5cccc6c5oc5ccccc56)ccc43)cc12 ; CC1(C)c2ccccc2-c2c1ccc1c2-c2ccccc2C12c1ccccc1-c1ccc(N(c3ccccc3)c3ccc(-c4ccccc4)cc3)cc12]

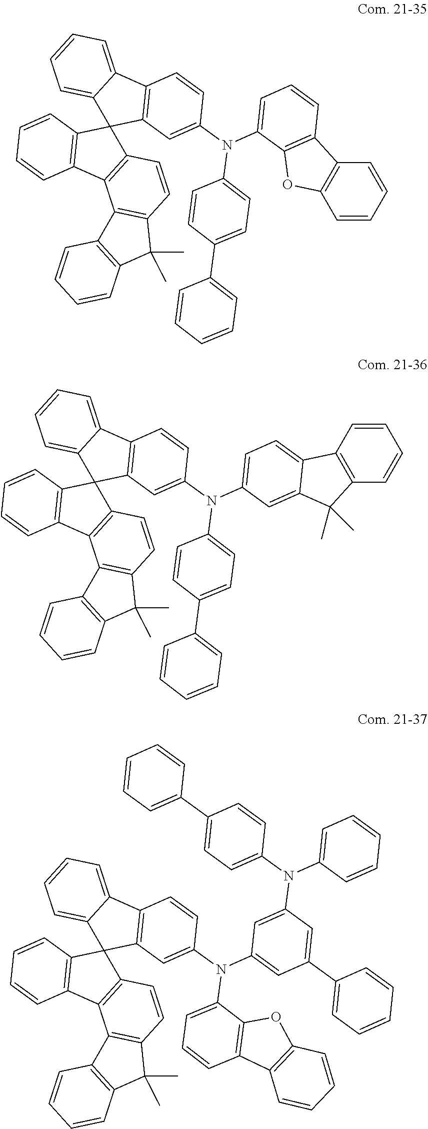 [SMILES: CC1(C)c2ccccc2-c2c1ccc1c2-c2ccccc2C12c1ccccc1-c1ccc(N(c3ccc(-c4ccccc4)cc3)c3cccc4c3oc3ccccc34)cc12 ; CC1(C)c2ccccc2-c2ccc(N(c3ccc(-c4ccccc4)cc3)c3ccc4c(c3)C3(c5ccccc5-4)c4ccccc4-c4c3ccc3c4-c4ccccc4C3(C)C)cc21 ; CC1(C)c2ccccc2-c2c1ccc1c2-c2ccccc2C12c1ccccc1-c1ccc(N(c3cc(-c4ccccc4)cc(N(c4ccccc4)c4ccc(-c5ccccc5)cc4)c3)c3cccc4c3oc3ccccc34)cc12]

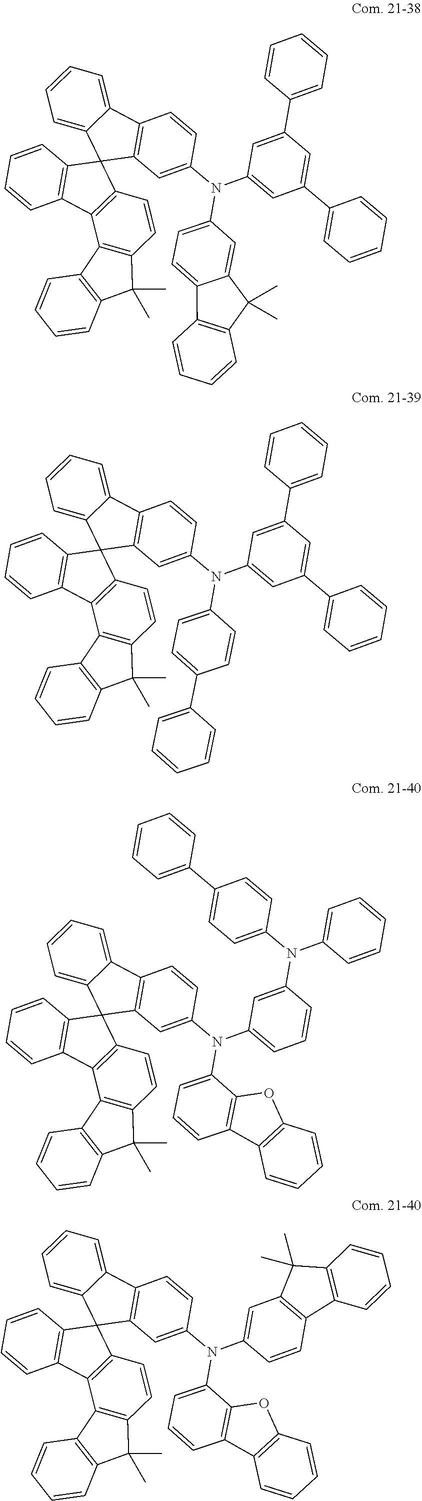

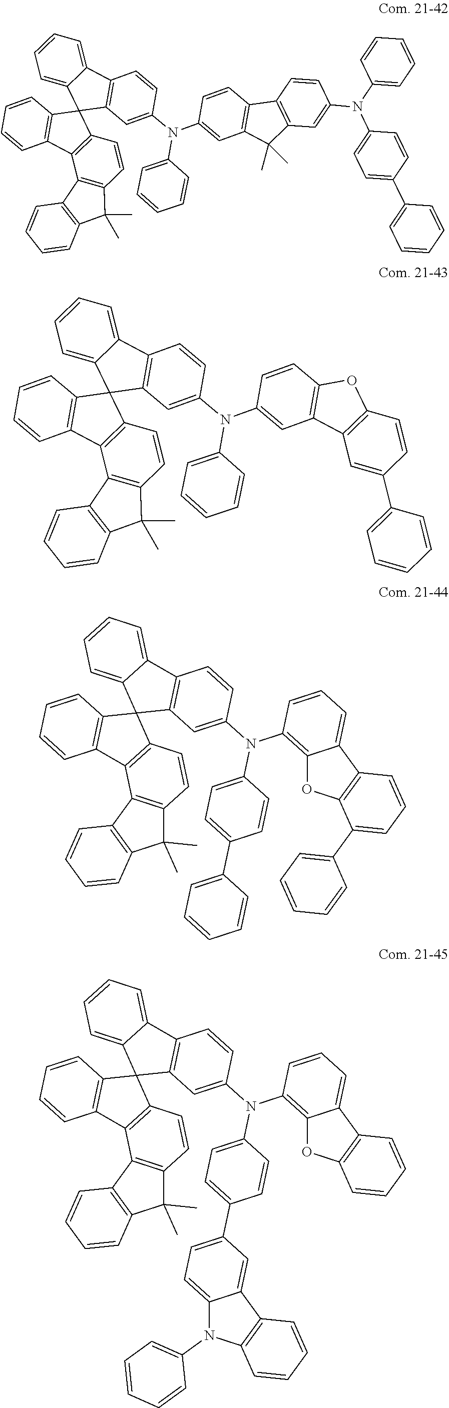 [SMILES: CC1(C)c2cc(N(c3ccccc3)c3ccc(-c4ccccc4)cc3)ccc2-c2ccc(N(c3ccccc3)c3ccc4c(c3)C3(c5ccccc5-4)c4ccccc4-c4c3ccc3c4-c4ccccc4C3(C)C)cc21 ; CC1(C)c2ccccc2-c2c1ccc1c2-c2ccccc2C12c1ccccc1-c1ccc(N(c3ccccc3)c3ccc4oc5ccc(-c6ccccc6)cc5c4c3)cc12 ; CC1(C)c2ccccc2-c2c1ccc1c2-c2ccccc2C12c1ccccc1-c1ccc(N(c3ccc(-c4ccccc4)cc3)c3cccc4c3oc3c(-c5ccccc5)cccc34)cc12 ; CC1(C)c2ccccc2-c2c1ccc1c2-c2ccccc2C12c1ccccc1-c1ccc(N(c3ccc(-c4ccc5c(c4)c4ccccc4n5-c4ccccc4)cc3)c3cccc4c3oc3ccccc34)cc12]

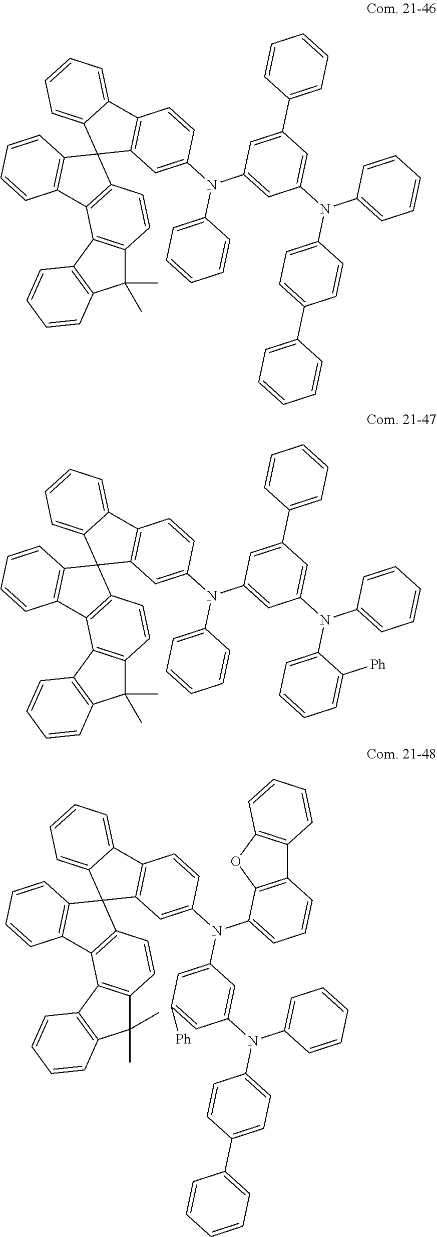

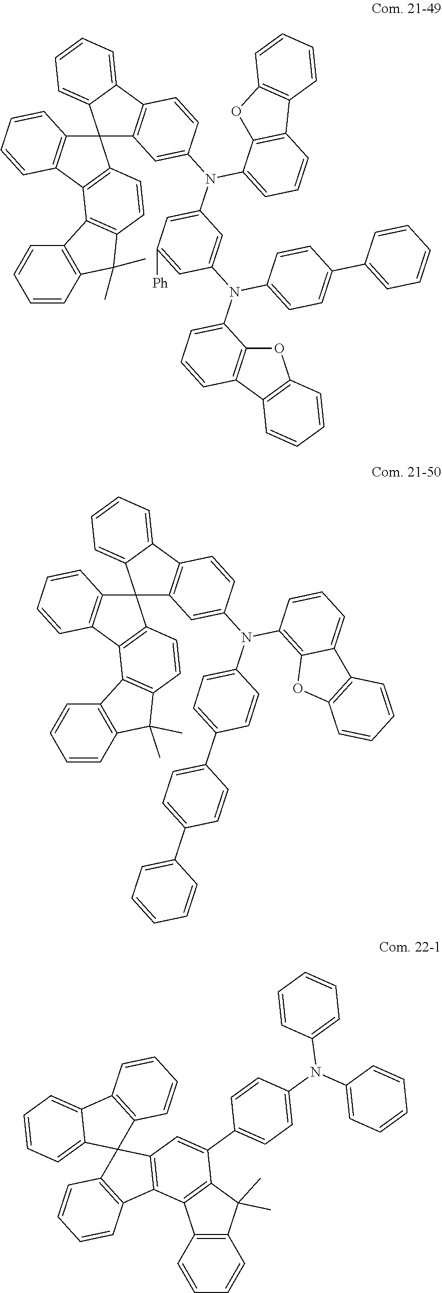 [SMILES: CC1(C)c2ccccc2-c2c1ccc1c2-c2ccccc2C12c1ccccc1-c1ccc(N(c3cc(-c4ccccc4)cc(N(c4ccc(-c5ccccc5)cc4)c4cccc5c4oc4ccccc45)c3)c3cccc4c3oc3ccccc34)cc12 ; CC1(C)c2ccccc2-c2c1ccc1c2-c2ccccc2C12c1ccccc1-c1ccc(N(c3ccc(-c4ccc(-c5ccccc5)cc4)cc3)c3cccc4c3oc3ccccc34)cc12 ; CC1(C)c2ccccc2-c2c3c(cc(-c4ccc(N(c5ccccc5)c5ccccc5)cc4)c21)C1(c2ccccc2-c2ccccc21)c1ccccc1-3]

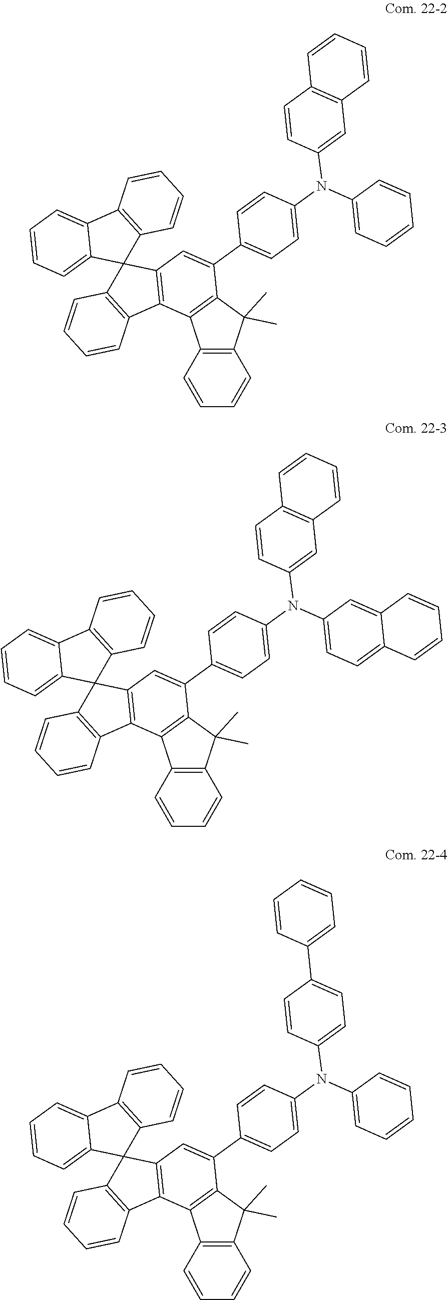 [SMILES: CC1(C)c2ccccc2-c2c3c(cc(-c4ccc(N(c5ccccc5)c5ccc6ccccc6c5)cc4)c21)C1(c2ccccc2-c2ccccc21)c1ccccc1-3 ; CC1(C)c2ccccc2-c2c3c(cc(-c4ccc(N(c5ccc6ccccc6c5)c5ccc6ccccc6c5)cc4)c21)C1(c2ccccc2-c2ccccc21)c1ccccc1-3 ; CC1(C)c2ccccc2-c2c3c(cc(-c4ccc(N(c5ccccc5)c5ccc(-c6ccccc6)cc5)cc4)c21)C1(c2ccccc2-c2ccccc21)c1ccccc1-3]

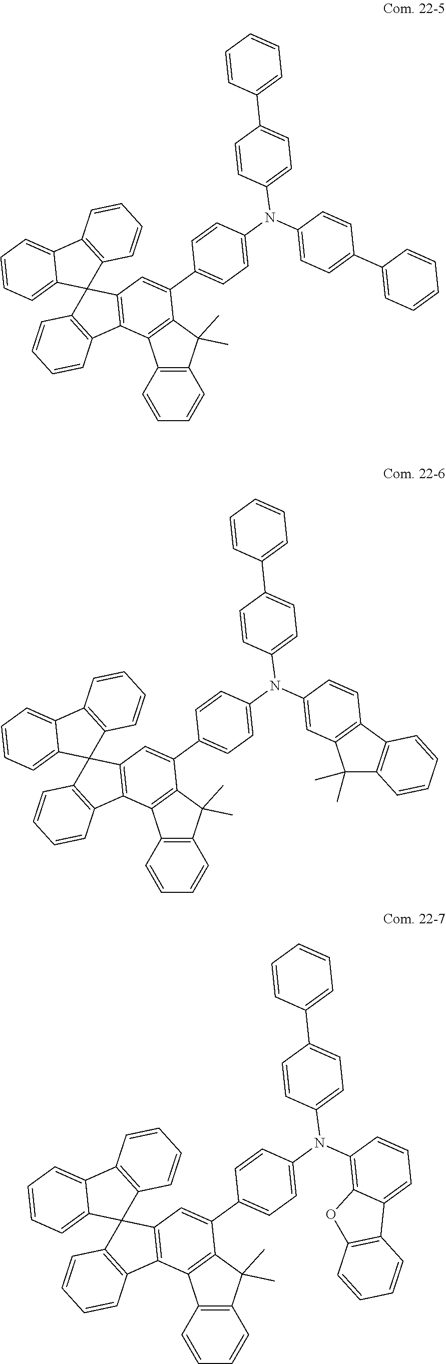 [SMILES: CC1(C)c2ccccc2-c2c3c(cc(-c4ccc(N(c5ccc(-c6ccccc6)cc5)c5ccc(-c6ccccc6)cc5)cc4)c21)C1(c2ccccc2-c2ccccc21)c1ccccc1-3 ; CC1(C)c2ccccc2-c2ccc(N(c3ccc(-c4ccccc4)cc3)c3ccc(-c4cc5c(c6c4C(C)(C)c4ccccc4-6)-c4ccccc4C54c5ccccc5-c5ccccc54)cc3)cc21 ; CC1(C)c2ccccc2-c2c3c(cc(-c4ccc(N(c5ccc(-c6ccccc6)cc5)c5cccc6c5oc5ccccc56)cc4)c21)C1(c2ccccc2-c2ccccc21)c1ccccc1-3]

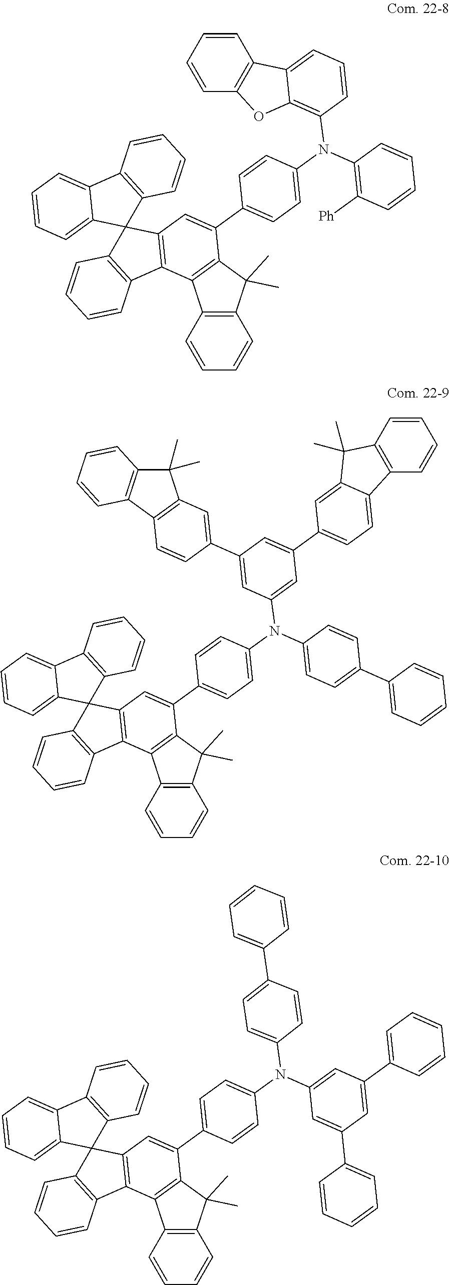 [SMILES: CC1(C)c2ccccc2-c2c3c(cc(-c4ccc(N(c5ccccc5-c5ccccc5)c5cccc6c5oc5ccccc56)cc4)c21)C1(c2ccccc2-c2ccccc21)c1ccccc1-3 ; CC1(C)c2ccccc2-c2ccc(-c3cc(-c4ccc5c(c4)C(C)(C)c4ccccc4-5)cc(N(c4ccc(-c5ccccc5)cc4)c4ccc(-c5cc6c(c7c5C(C)(C)c5ccccc5-7)-c5ccccc5C65c6ccccc6-c6ccccc65)cc4)c3)cc21 ; CC1(C)c2ccccc2-c2c3c(cc(-c4ccc(N(c5ccc(-c6ccccc6)cc5)c5cc(-c6ccccc6)cc(-c6ccccc6)c5)cc4)c21)C1(c2ccccc2-c2ccccc21)c1ccccc1-3]

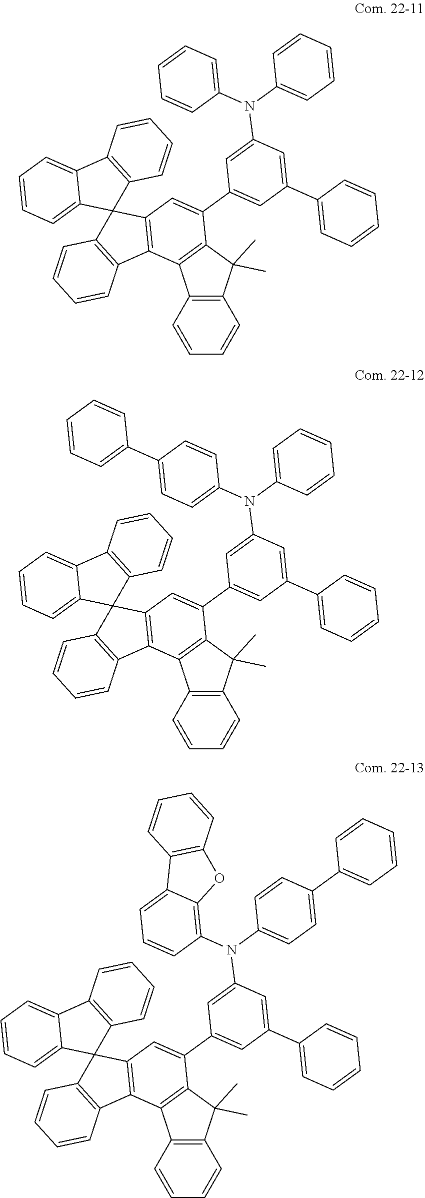

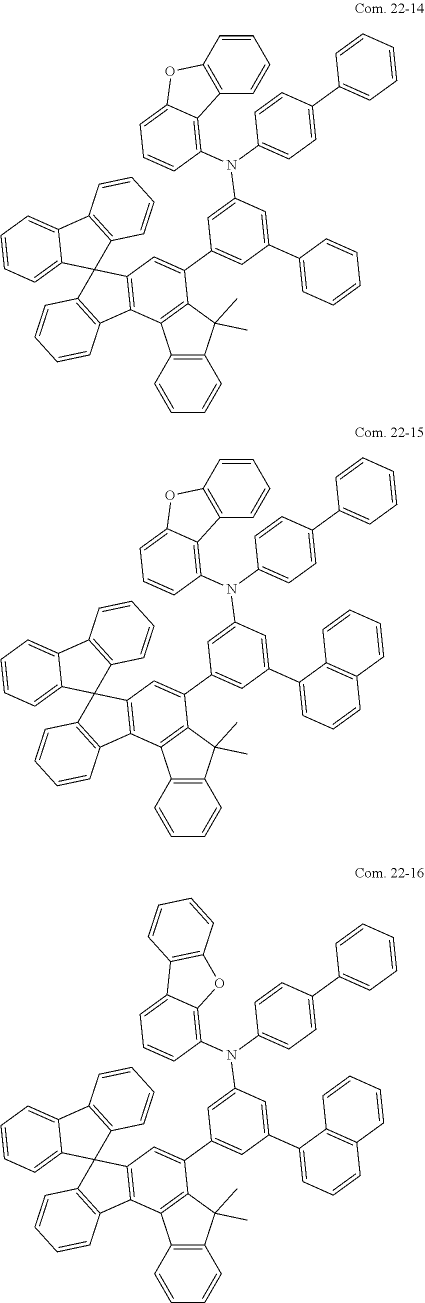 [SMILES: CC1(C)c2ccccc2-c2c3c(cc(-c4cc(-c5ccccc5)cc(N(c5ccc(-c6ccccc6)cc5)c5cccc6oc7ccccc7c56)c4)c21)C1(c2ccccc2-c2ccccc21)c1ccccc1-3 ; CC1(C)c2ccccc2-c2c3c(cc(-c4cc(-c5cccc6ccccc56)cc(N(c5ccc(-c6ccccc6)cc5)c5cccc6oc7ccccc7c56)c4)c21)C1(c2ccccc2-c2ccccc21)c1ccccc1-3 ; CC1(C)c2ccccc2-c2c3c(cc(-c4cc(-c5cccc6ccccc56)cc(N(c5ccc(-c6ccccc6)cc5)c5cccc6c5oc5ccccc56)c4)c21)C1(c2ccccc2-c2ccccc21)c1ccccc1-3]

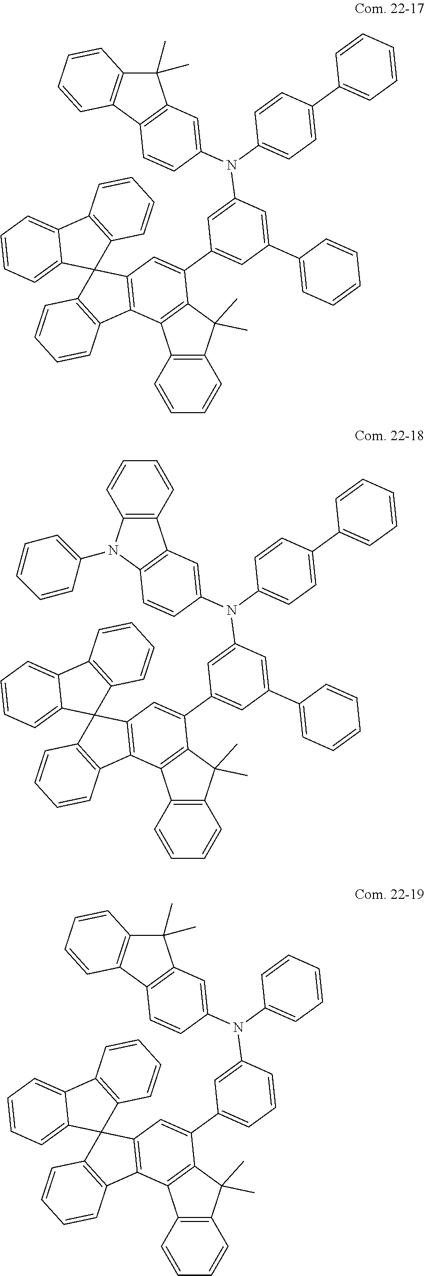

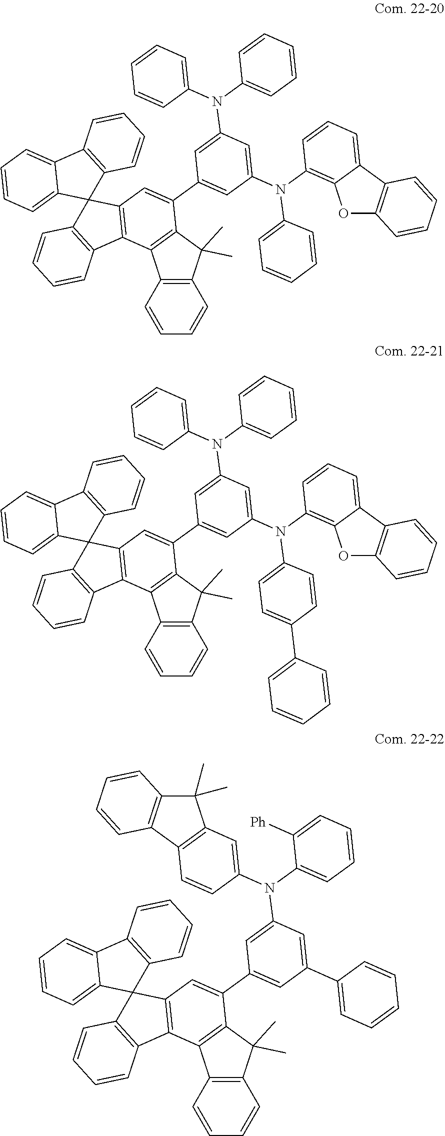 [SMILES: CC1(C)c2ccccc2-c2c3c(cc(-c4cc(N(c5ccccc5)c5ccccc5)cc(N(c5ccccc5)c5cccc6c5oc5ccccc56)c4)c21)C1(c2ccccc2-c2ccccc21)c1ccccc1-3 ; CC1(C)c2ccccc2-c2c3c(cc(-c4cc(N(c5ccccc5)c5ccccc5)cc(N(c5ccc(-c6ccccc6)cc5)c5cccc6c5oc5ccccc56)c4)c21)C1(c2ccccc2-c2ccccc21)c1ccccc1-3 ; CC1(C)c2ccccc2-c2ccc(N(c3cc(-c4ccccc4)cc(-c4cc5c(c6c4C(C)(C)c4ccccc4-6)-c4ccccc4C54c5ccccc5-c5ccccc54)c3)c3ccccc3-c3ccccc3)cc21]

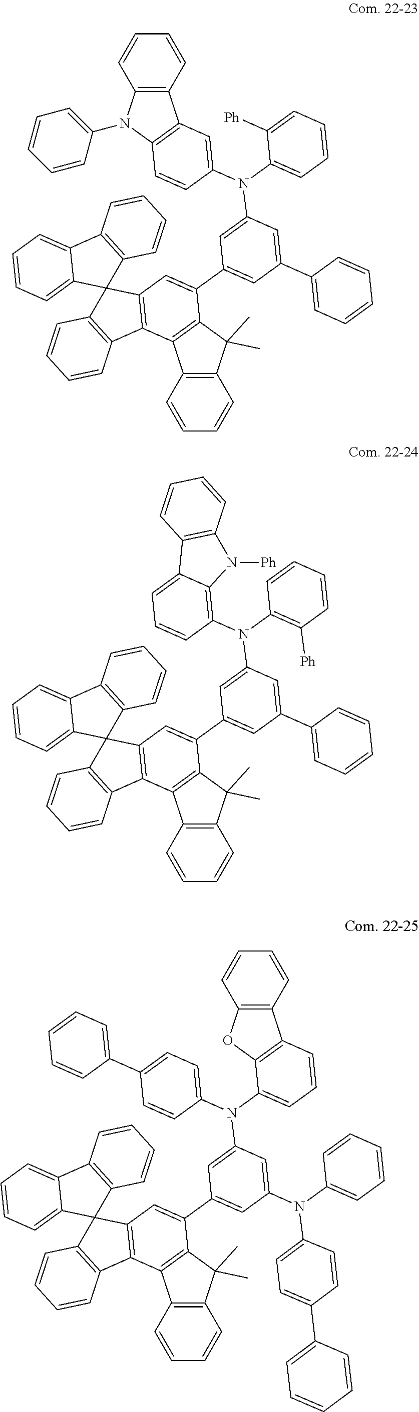 [SMILES: CC1(C)c2ccccc2-c2c3c(cc(-c4cc(-c5ccccc5)cc(N(c5ccc6c(c5)c5ccccc5n6-c5ccccc5)c5ccccc5-c5ccccc5)c4)c21)C1(c2ccccc2-c2ccccc21)c1ccccc1-3 ; CC1(C)c2ccccc2-c2c3c(cc(-c4cc(-c5ccccc5)cc(N(c5ccccc5-c5ccccc5)c5cccc6c7ccccc7n(-c7ccccc7)c56)c4)c21)C1(c2ccccc2-c2ccccc21)c1ccccc1-3 ; CC1(C)c2ccccc2-c2c3c(cc(-c4cc(N(c5ccccc5)c5ccc(-c6ccccc6)cc5)cc(N(c5ccc(-c6ccccc6)cc5)c5cccc6c5oc5ccccc56)c4)c21)C1(c2ccccc2-c2ccccc21)c1ccccc1-3]

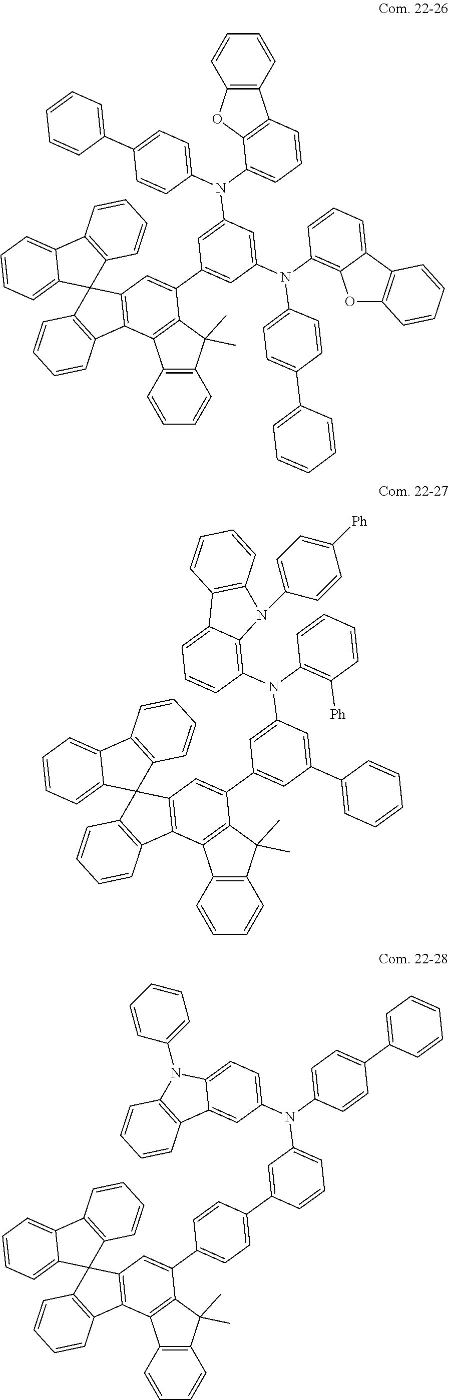 [SMILES: CC1(C)c2ccccc2-c2c3c(cc(-c4cc(N(c5ccc(-c6ccccc6)cc5)c5cccc6c5oc5ccccc56)cc(N(c5ccc(-c6ccccc6)cc5)c5cccc6c5oc5ccccc56)c4)c21)C1(c2ccccc2-c2ccccc21)c1ccccc1-3 ; CC1(C)c2ccccc2-c2c3c(cc(-c4cc(-c5ccccc5)cc(N(c5ccccc5-c5ccccc5)c5cccc6c7ccccc7n(-c7ccc(-c8ccccc8)cc7)c56)c4)c21)C1(c2ccccc2-c2ccccc21)c1ccccc1-3 ; CC1(C)c2ccccc2-c2c3c(cc(-c4ccc(-c5cccc(N(c6ccc(-c7ccccc7)cc6)c6ccc7c(c6)c6ccccc6n7-c6ccccc6)c5)cc4)c21)C1(c2ccccc2-c2ccccc21)c1ccccc1-3]

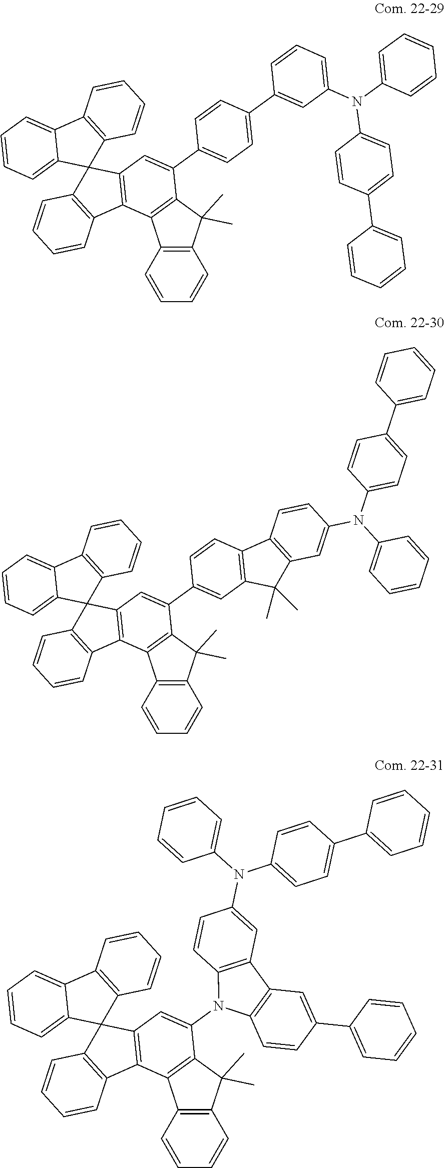 [SMILES: CC1(C)c2ccccc2-c2c3c(cc(-c4ccc(-c5cccc(N(c6ccccc6)c6ccc(-c7ccccc7)cc6)c5)cc4)c21)C1(c2ccccc2-c2ccccc21)c1ccccc1-3 ; CC1(C)c2cc(-c3cc4c(c5c3C(C)(C)c3ccccc3-5)-c3ccccc3C43c4ccccc4-c4ccccc43)ccc2-c2ccc(N(c3ccccc3)c3ccc(-c4ccccc4)cc3)cc21 ; CC1(C)c2ccccc2-c2c3c(cc(-n4c5ccc(-c6ccccc6)cc5c5cc(N(c6ccccc6)c6ccc(-c7ccccc7)cc6)ccc54)c21)C1(c2ccccc2-c2ccccc21)c1ccccc1-3]

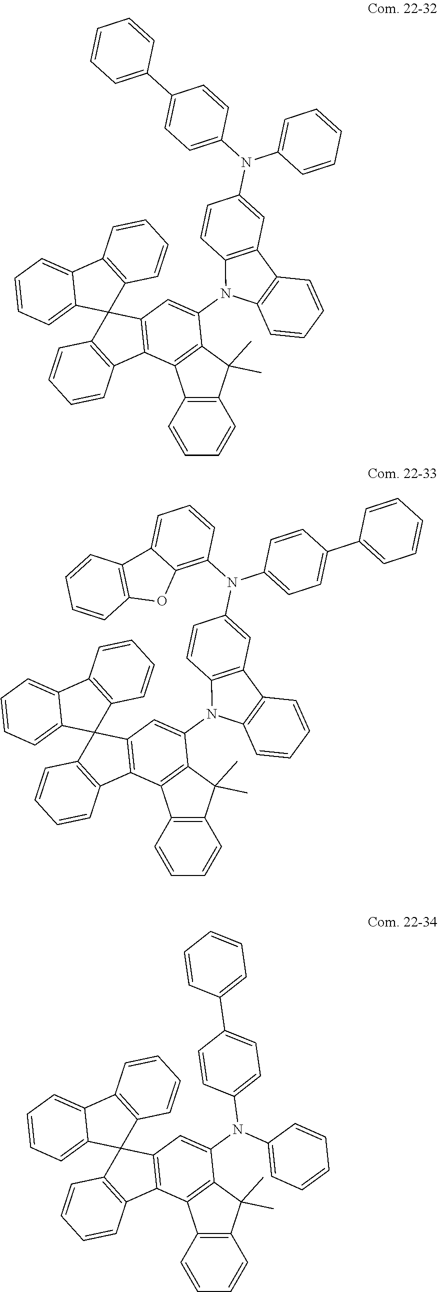 [SMILES: CC1(C)c2ccccc2-c2c3c(cc(-n4c5ccccc5c5cc(N(c6ccccc6)c6ccc(-c7ccccc7)cc6)ccc54)c21)C1(c2ccccc2-c2ccccc21)c1ccccc1-3 ; CC1(C)c2ccccc2-c2c3c(cc(-n4c5ccccc5c5cc(N(c6ccc(-c7ccccc7)cc6)c6cccc7c6oc6ccccc67)ccc54)c21)C1(c2ccccc2-c2ccccc21)c1ccccc1-3 ; CC1(C)c2ccccc2-c2c3c(cc(N(c4ccccc4)c4ccc(-c5ccccc5)cc4)c21)C1(c2ccccc2-c2ccccc21)c1ccccc1-3]

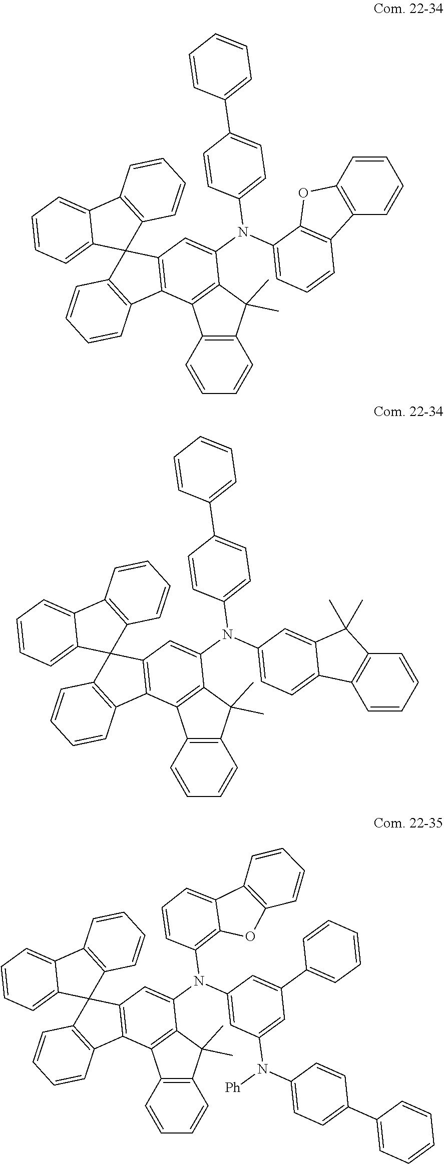

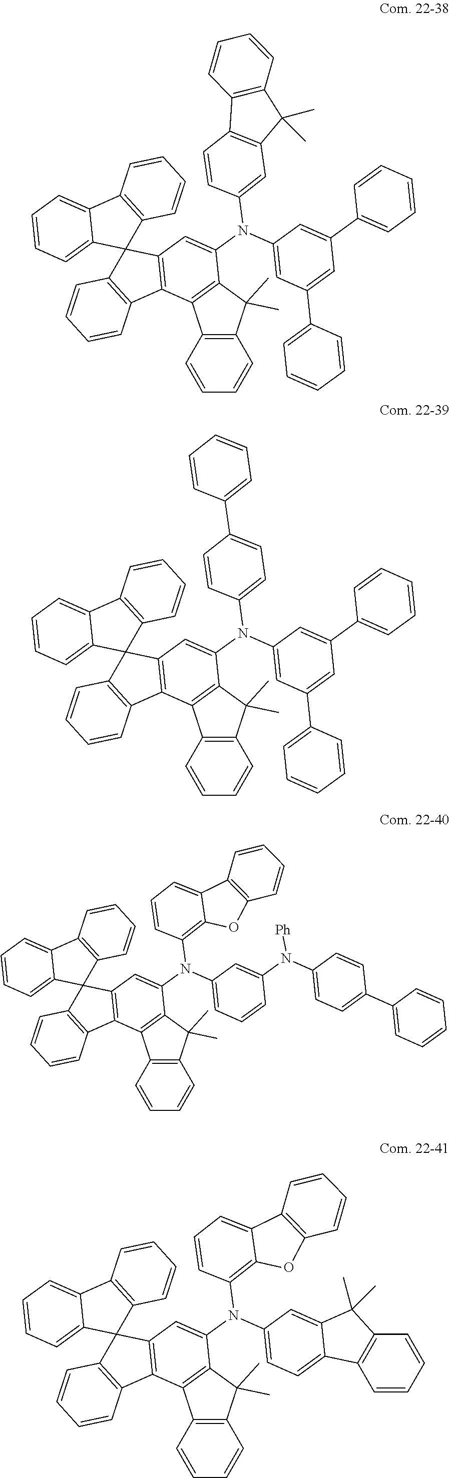

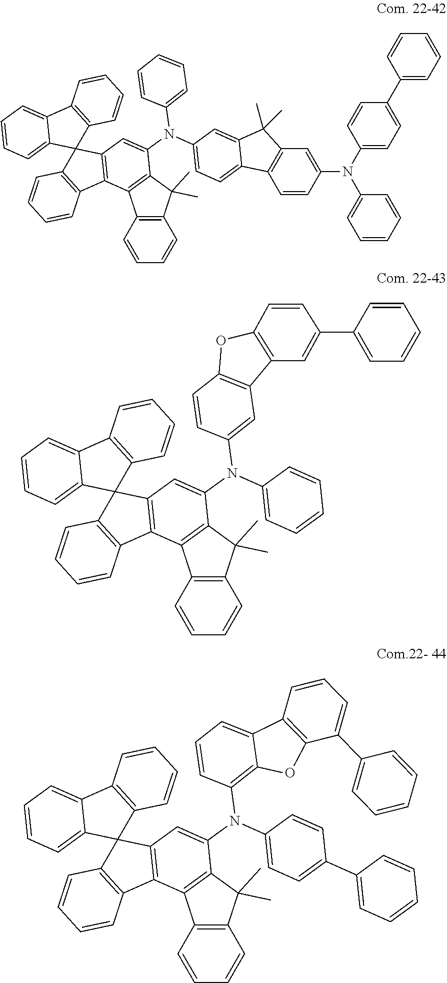 [SMILES: CC1(C)c2cc(N(c3ccccc3)c3ccc(-c4ccccc4)cc3)ccc2-c2ccc(N(c3ccccc3)c3cc4c(c5c3C(C)(C)c3ccccc3-5)-c3ccccc3C43c4ccccc4-c4ccccc43)cc21 ; CC1(C)c2ccccc2-c2c3c(cc(N(c4ccccc4)c4ccc5oc6ccc(-c7ccccc7)cc6c5c4)c21)C1(c2ccccc2-c2ccccc21)c1ccccc1-3 ; CC1(C)c2ccccc2-c2c3c(cc(N(c4ccc(-c5ccccc5)cc4)c4cccc5c4oc4c(-c6ccccc6)cccc45)c21)C1(c2ccccc2-c2ccccc21)c1ccccc1-3]

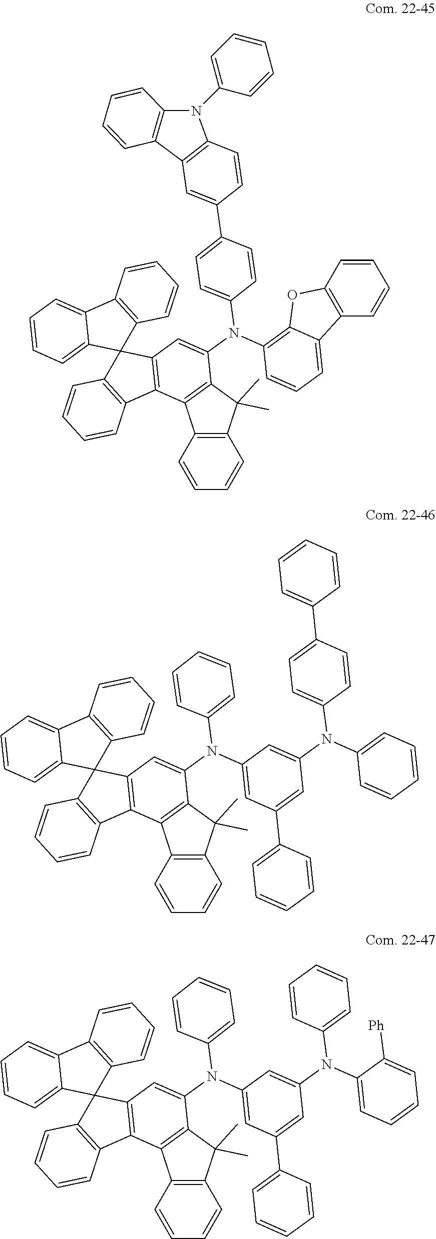 [SMILES: CC1(C)c2ccccc2-c2c3c(cc(N(c4ccc(-c5ccc6c(c5)c5ccccc5n6-c5ccccc5)cc4)c4cccc5c4oc4ccccc45)c21)C1(c2ccccc2-c2ccccc21)c1ccccc1-3 ; CC1(C)c2ccccc2-c2c3c(cc(N(c4ccccc4)c4cc(-c5ccccc5)cc(N(c5ccccc5)c5ccc(-c6ccccc6)cc5)c4)c21)C1(c2ccccc2-c2ccccc21)c1ccccc1-3 ; CC1(C)c2ccccc2-c2c3c(cc(N(c4ccccc4)c4cc(-c5ccccc5)cc(N(c5ccccc5)c5ccccc5-c5ccccc5)c4)c21)C1(c2ccccc2-c2ccccc21)c1ccccc1-3]

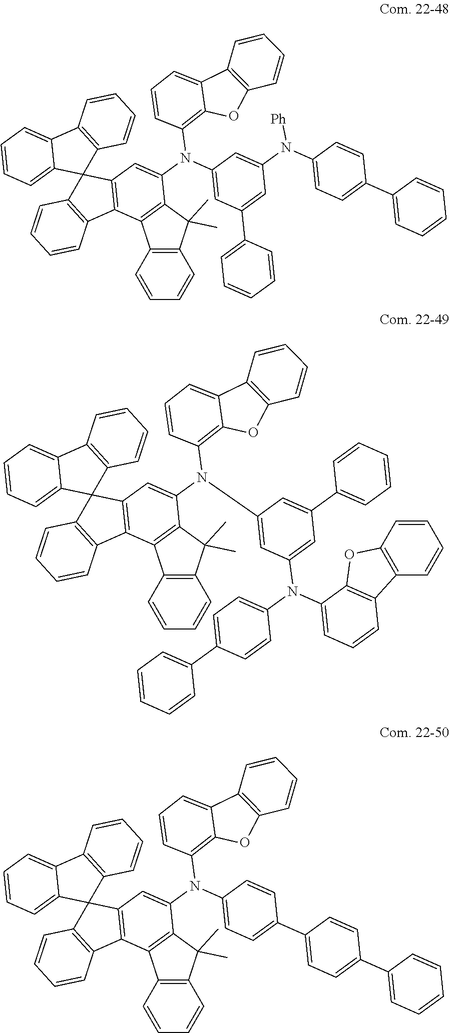 [SMILES: CC1(C)c2ccccc2-c2c3c(cc(N(c4cc(-c5ccccc5)cc(N(c5ccccc5)c5ccc(-c6ccccc6)cc5)c4)c4cccc5c4oc4ccccc45)c21)C1(c2ccccc2-c2ccccc21)c1ccccc1-3 ; CC1(C)c2ccccc2-c2c3c(cc(N(c4cc(-c5ccccc5)cc(N(c5ccc(-c6ccccc6)cc5)c5cccc6c5oc5ccccc56)c4)c4cccc5c4oc4ccccc45)c21)C1(c2ccccc2-c2ccccc21)c1ccccc1-3 ; CC1(C)c2ccccc2-c2c3c(cc(N(c4ccc(-c5ccc(-c6ccccc6)cc5)cc4)c4cccc5c4oc4ccccc45)c21)C1(c2ccccc2-c2ccccc21)c1ccccc1-3]

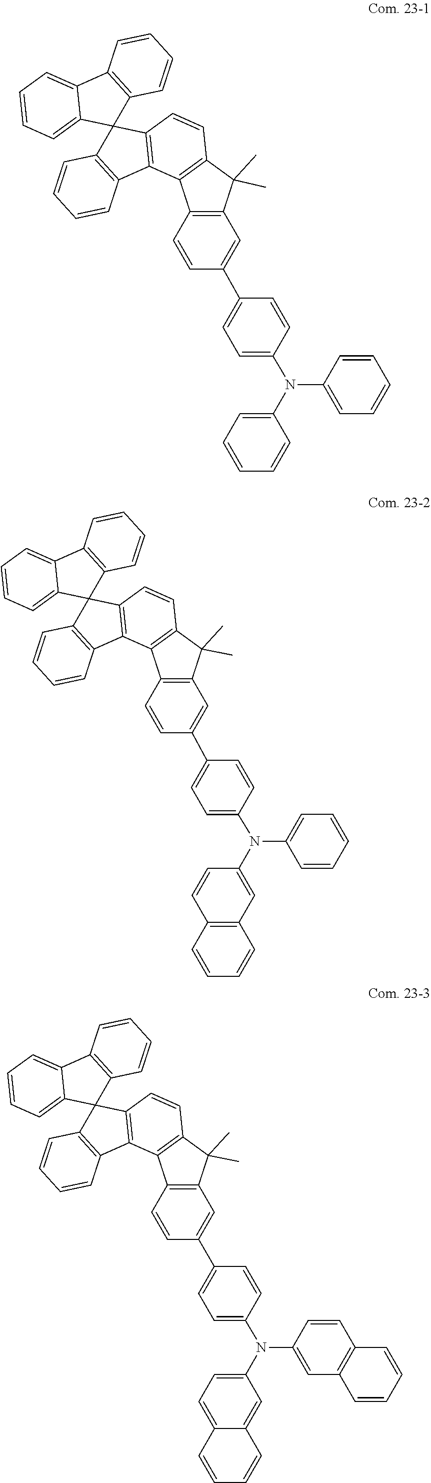 [SMILES: CC1(C)c2cc(-c3ccc(N(c4ccccc4)c4ccccc4)cc3)ccc2-c2c1ccc1c2-c2ccccc2C12c1ccccc1-c1ccccc12 ; CC1(C)c2cc(-c3ccc(N(c4ccccc4)c4ccc5ccccc5c4)cc3)ccc2-c2c1ccc1c2-c2ccccc2C12c1ccccc1-c1ccccc12 ; CC1(C)c2cc(-c3ccc(N(c4ccc5ccccc5c4)c4ccc5ccccc5c4)cc3)ccc2-c2c1ccc1c2-c2ccccc2C12c1ccccc1-c1ccccc12]

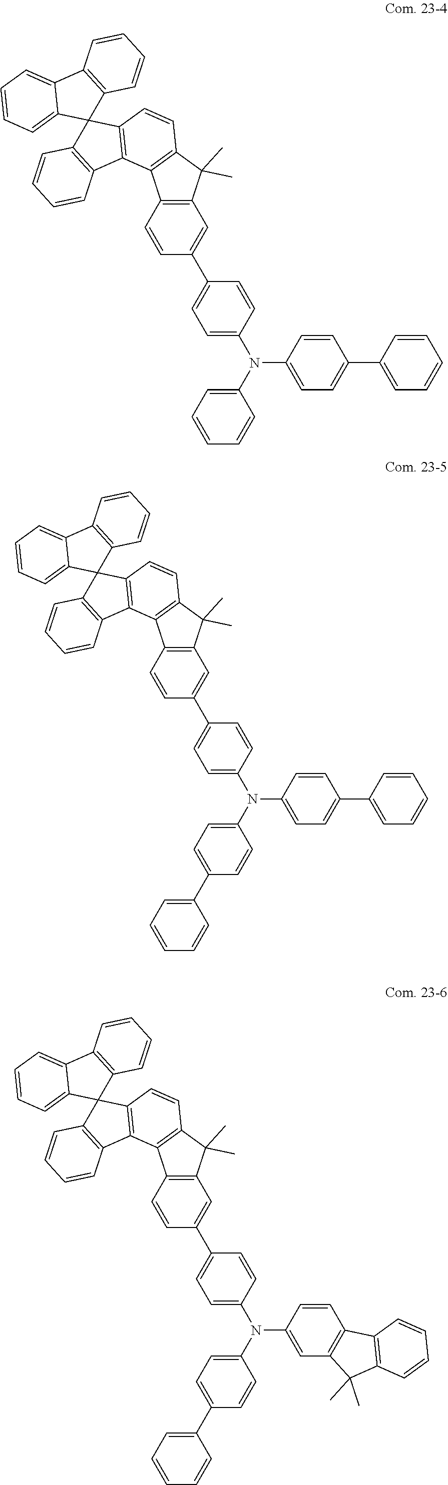 [SMILES: CC1(C)c2cc(-c3ccc(N(c4ccccc4)c4ccc(-c5ccccc5)cc4)cc3)ccc2-c2c1ccc1c2-c2ccccc2C12c1ccccc1-c1ccccc12 ; CC1(C)c2cc(-c3ccc(N(c4ccc(-c5ccccc5)cc4)c4ccc(-c5ccccc5)cc4)cc3)ccc2-c2c1ccc1c2-c2ccccc2C12c1ccccc1-c1ccccc12 ; CC1(C)c2ccccc2-c2ccc(N(c3ccc(-c4ccccc4)cc3)c3ccc(-c4ccc5c(c4)C(C)(C)c4ccc6c(c4-5)-c4ccccc4C64c5ccccc5-c5ccccc54)cc3)cc21]

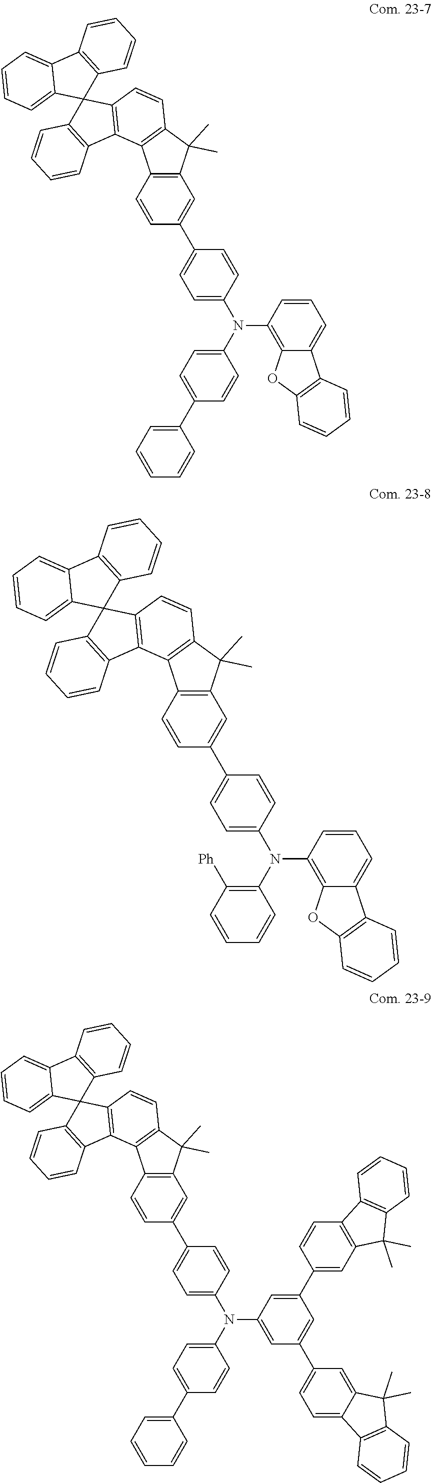 [SMILES: CC1(C)c2cc(-c3ccc(N(c4ccc(-c5ccccc5)cc4)c4cccc5c4oc4ccccc45)cc3)ccc2-c2c1ccc1c2-c2ccccc2C12c1ccccc1-c1ccccc12 ; CC1(C)c2cc(-c3ccc(N(c4ccccc4-c4ccccc4)c4cccc5c4oc4ccccc45)cc3)ccc2-c2c1ccc1c2-c2ccccc2C12c1ccccc1-c1ccccc12 ; CC1(C)c2ccccc2-c2ccc(-c3cc(-c4ccc5c(c4)C(C)(C)c4ccccc4-5)cc(N(c4ccc(-c5ccccc5)cc4)c4ccc(-c5ccc6c(c5)C(C)(C)c5ccc7c(c5-6)-c5ccccc5C75c6ccccc6-c6ccccc65)cc4)c3)cc21]

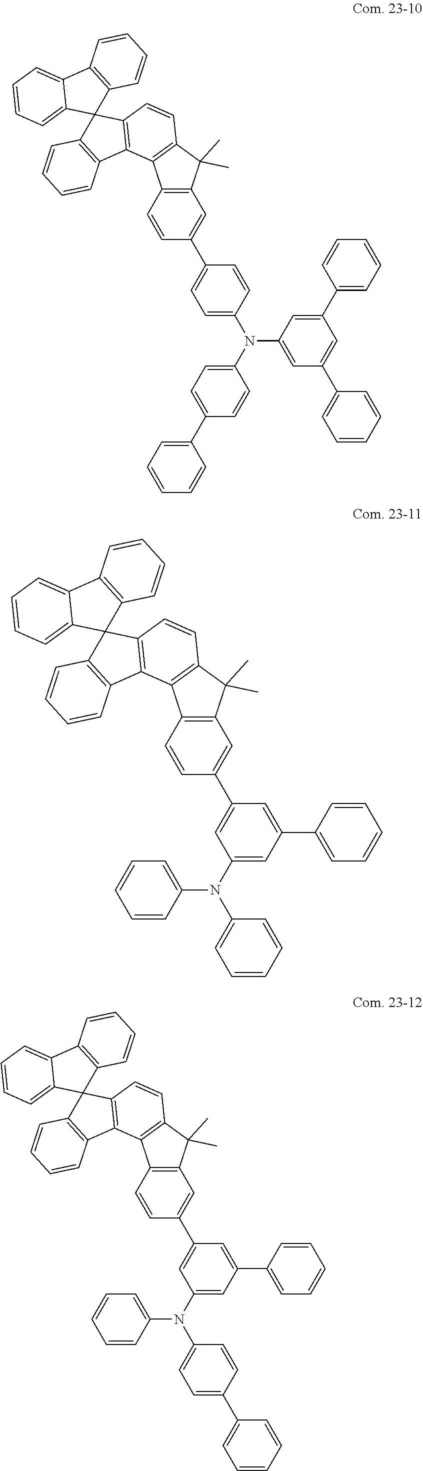 [SMILES: CC1(C)c2cc(-c3ccc(N(c4ccc(-c5ccccc5)cc4)c4cc(-c5ccccc5)cc(-c5ccccc5)c4)cc3)ccc2-c2c1ccc1c2-c2ccccc2C12c1ccccc1-c1ccccc12 ; CC1(C)c2cc(-c3cc(-c4ccccc4)cc(N(c4ccccc4)c4ccccc4)c3)ccc2-c2c1ccc1c2-c2ccccc2C12c1ccccc1-c1ccccc12 ; CC1(C)c2cc(-c3cc(-c4ccccc4)cc(N(c4ccccc4)c4ccc(-c5ccccc5)cc4)c3)ccc2-c2c1ccc1c2-c2ccccc2C12c1ccccc1-c1ccccc12]

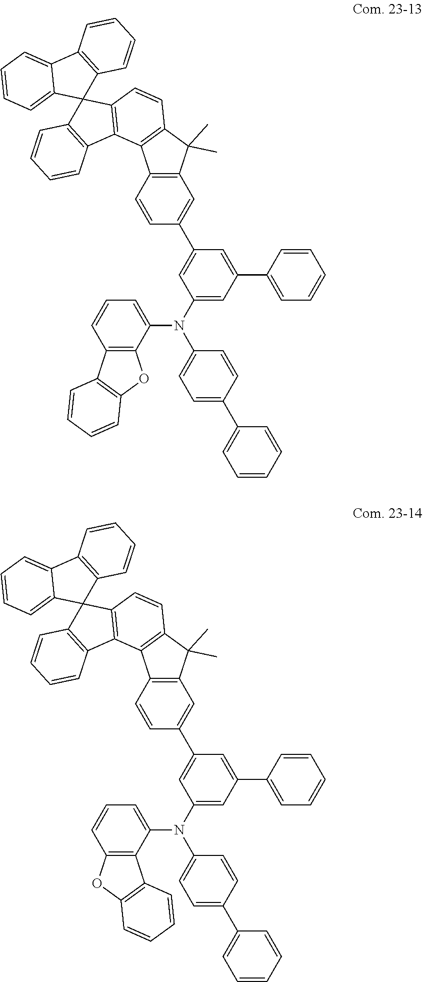 [SMILES: CC1(C)c2cc(-c3cc(-c4ccccc4)cc(N(c4ccc(-c5ccccc5)cc4)c4cccc5c4oc4ccccc45)c3)ccc2-c2c1ccc1c2-c2ccccc2C12c1ccccc1-c1ccccc12 ; CC1(C)c2cc(-c3cc(-c4ccccc4)cc(N(c4ccc(-c5ccccc5)cc4)c4cccc5oc6ccccc6c45)c3)ccc2-c2c1ccc1c2-c2ccccc2C12c1ccccc1-c1ccccc12]

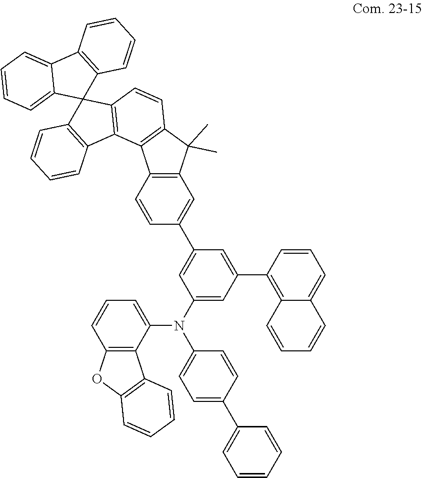 [SMILES: CC1(C)c2cc(-c3cc(-c4cccc5ccccc45)cc(N(c4ccc(-c5ccccc5)cc4)c4cccc5oc6ccccc6c45)c3)ccc2-c2c1ccc1c2-c2ccccc2C12c1ccccc1-c1ccccc12]

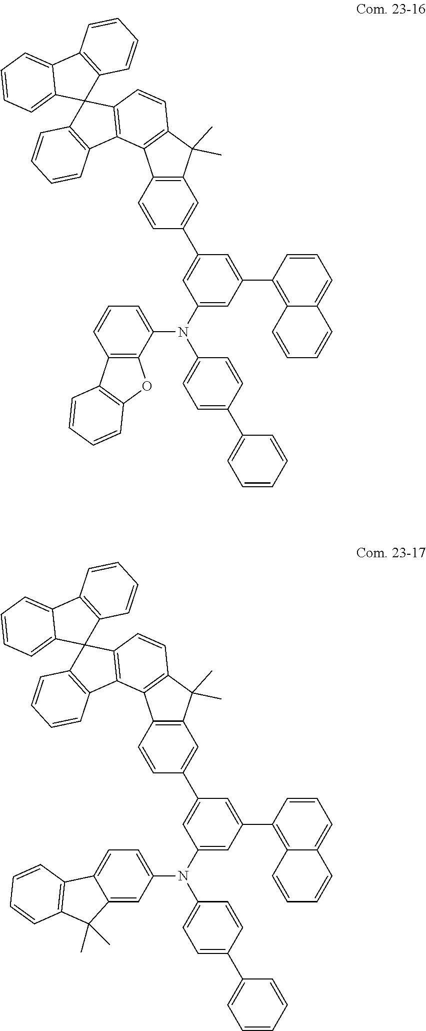 [SMILES: CC1(C)c2cc(-c3cc(-c4cccc5ccccc45)cc(N(c4ccc(-c5ccccc5)cc4)c4cccc5c4oc4ccccc45)c3)ccc2-c2c1ccc1c2-c2ccccc2C12c1ccccc1-c1ccccc12 ; CC1(C)c2ccccc2-c2ccc(N(c3ccc(-c4ccccc4)cc3)c3cc(-c4ccc5c(c4)C(C)(C)c4ccc6c(c4-5)-c4ccccc4C64c5ccccc5-c5ccccc54)cc(-c4cccc5ccccc45)c3)cc21]

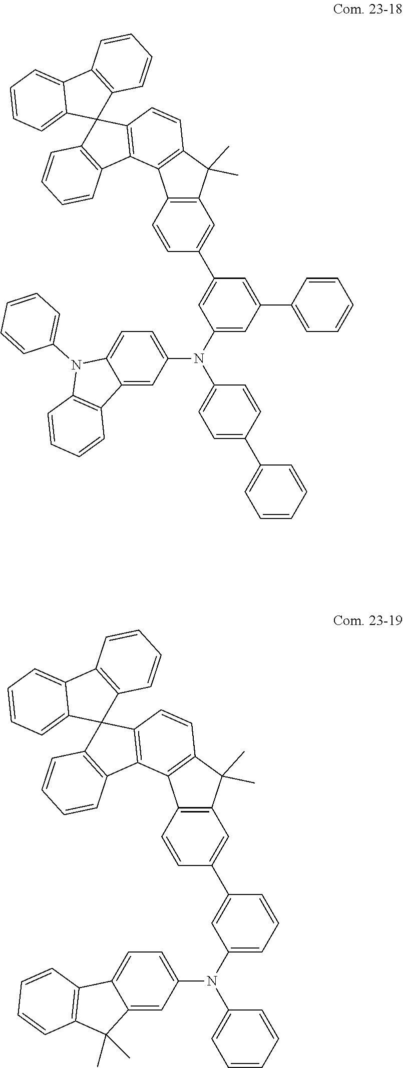 [SMILES: CC1(C)c2cc(-c3cc(-c4ccccc4)cc(N(c4ccc(-c5ccccc5)cc4)c4ccc5c(c4)c4ccccc4n5-c4ccccc4)c3)ccc2-c2c1ccc1c2-c2ccccc2C12c1ccccc1-c1ccccc12 ; CC1(C)c2ccccc2-c2ccc(N(c3ccccc3)c3cccc(-c4ccc5c(c4)C(C)(C)c4ccc6c(c4-5)-c4ccccc4C64c5ccccc5-c5ccccc54)c3)cc21]

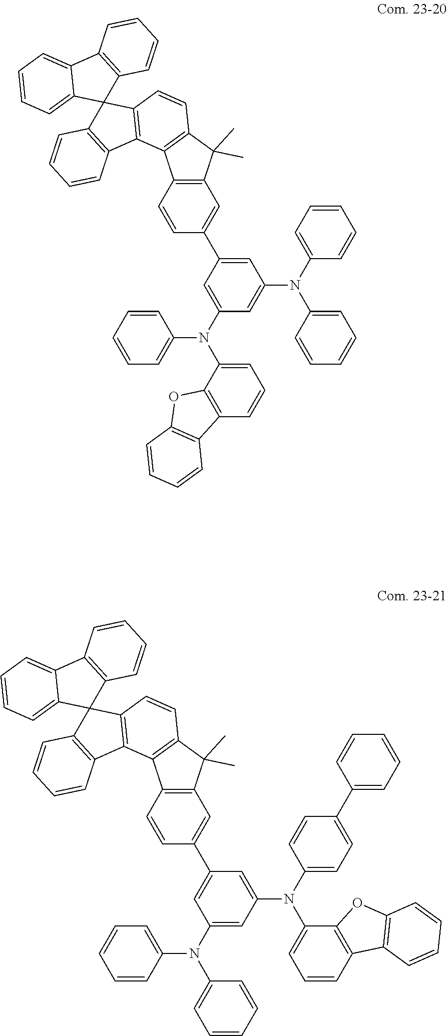 [SMILES: CC1(C)c2cc(-c3cc(N(c4ccccc4)c4ccccc4)cc(N(c4ccccc4)c4cccc5c4oc4ccccc45)c3)ccc2-c2c1ccc1c2-c2ccccc2C12c1ccccc1-c1ccccc12 ; CC1(C)c2cc(-c3cc(N(c4ccccc4)c4ccccc4)cc(N(c4ccc(-c5ccccc5)cc4)c4cccc5c4oc4ccccc45)c3)ccc2-c2c1ccc1c2-c2ccccc2C12c1ccccc1-c1ccccc12]

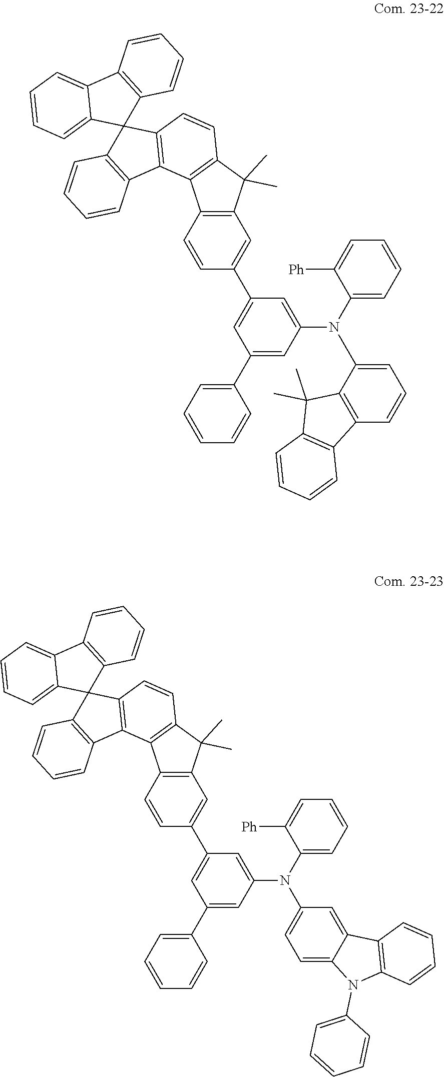 [SMILES: CC1(C)c2cc(-c3cc(-c4ccccc4)cc(N(c4ccccc4-c4ccccc4)c4cccc5c4C(C)(C)c4ccccc4-5)c3)ccc2-c2c1ccc1c2-c2ccccc2C12c1ccccc1-c1ccccc12 ; CC1(C)c2cc(-c3cc(-c4ccccc4)cc(N(c4ccc5c(c4)c4ccccc4n5-c4ccccc4)c4ccccc4-c4ccccc4)c3)ccc2-c2c1ccc1c2-c2ccccc2C12c1ccccc1-c1ccccc12]

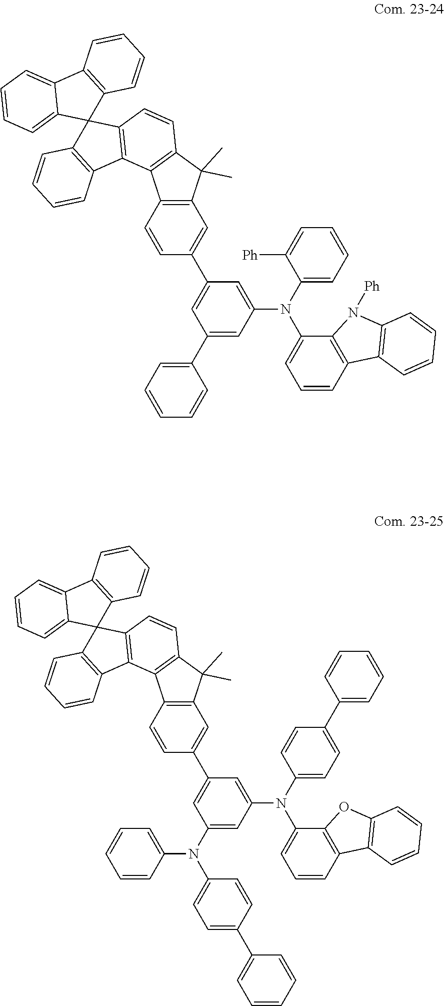 [SMILES: CC1(C)c2cc(-c3cc(-c4ccccc4)cc(N(c4ccccc4-c4ccccc4)c4cccc5c6ccccc6n(-c6ccccc6)c45)c3)ccc2-c2c1ccc1c2-c2ccccc2C12c1ccccc1-c1ccccc12 ; CC1(C)c2cc(-c3cc(N(c4ccccc4)c4ccc(-c5ccccc5)cc4)cc(N(c4ccc(-c5ccccc5)cc4)c4cccc5c4oc4ccccc45)c3)ccc2-c2c1ccc1c2-c2ccccc2C12c1ccccc1-c1ccccc12]

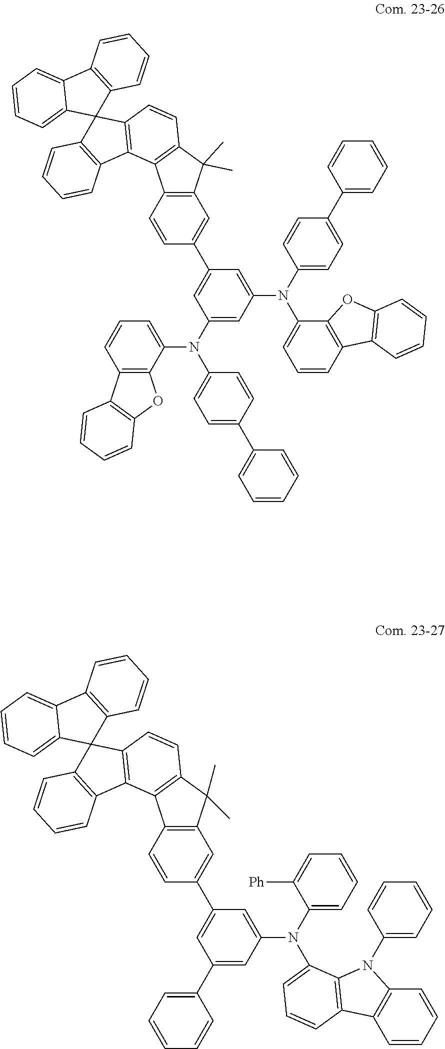 [SMILES: CC1(C)c2cc(-c3cc(N(c4ccc(-c5ccccc5)cc4)c4cccc5c4oc4ccccc45)cc(N(c4ccc(-c5ccccc5)cc4)c4cccc5c4oc4ccccc45)c3)ccc2-c2c1ccc1c2-c2ccccc2C12c1ccccc1-c1ccccc12 ; CC1(C)c2cc(-c3cc(-c4ccccc4)cc(N(c4ccccc4-c4ccccc4)c4cccc5c6ccccc6n(-c6ccccc6)c45)c3)ccc2-c2c1ccc1c2-c2ccccc2C12c1ccccc1-c1ccccc12]

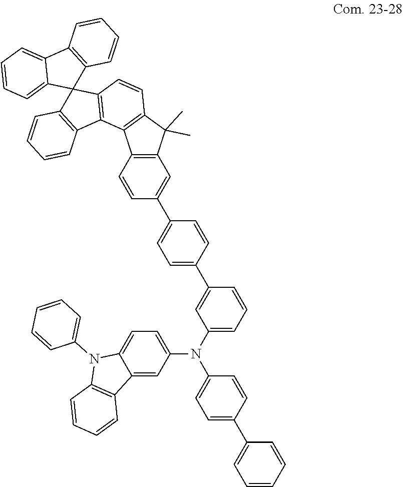 [SMILES: CC1(C)c2cc(-c3ccc(-c4cccc(N(c5ccc(-c6ccccc6)cc5)c5ccc6c(c5)c5ccccc5n6-c5ccccc5)c4)cc3)ccc2-c2c1ccc1c2-c2ccccc2C12c1ccccc1-c1ccccc12]

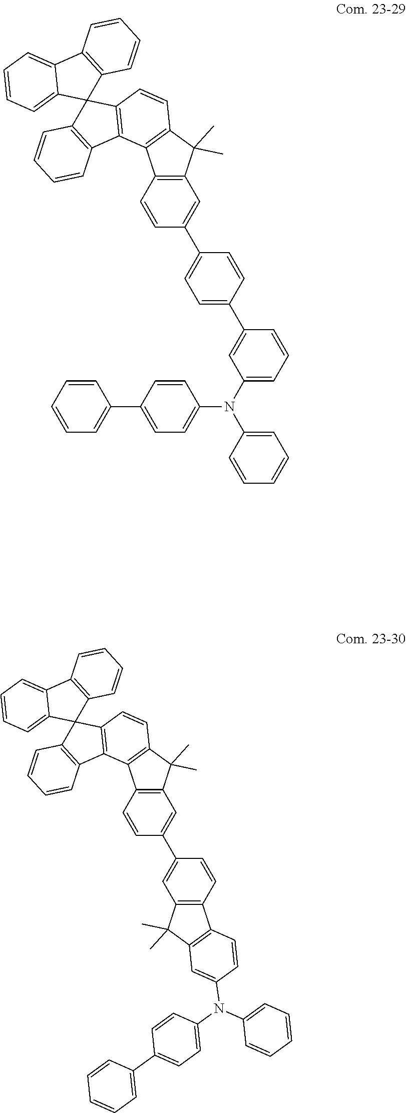 [SMILES: CC1(C)c2cc(-c3ccc(-c4cccc(N(c5ccccc5)c5ccc(-c6ccccc6)cc5)c4)cc3)ccc2-c2c1ccc1c2-c2ccccc2C12c1ccccc1-c1ccccc12 ; CC1(C)c2cc(-c3ccc4c(c3)C(C)(C)c3ccc5c(c3-4)-c3ccccc3C53c4ccccc4-c4ccccc43)ccc2-c2ccc(N(c3ccccc3)c3ccc(-c4ccccc4)cc3)cc21]

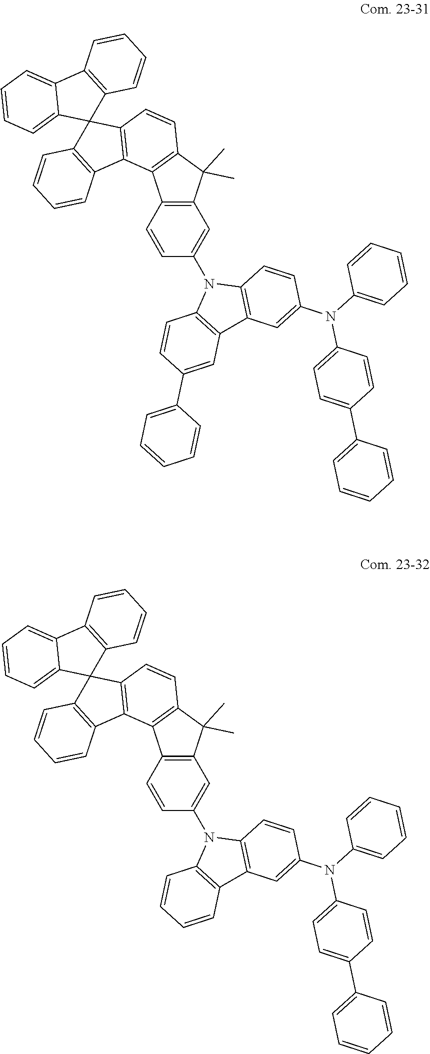 [SMILES: CC1(C)c2cc(-n3c4ccc(-c5ccccc5)cc4c4cc(N(c5ccccc5)c5ccc(-c6ccccc6)cc5)ccc43)ccc2-c2c1ccc1c2-c2ccccc2C12c1ccccc1-c1ccccc12 ; CC1(C)c2cc(-n3c4ccccc4c4cc(N(c5ccccc5)c5ccc(-c6ccccc6)cc5)ccc43)ccc2-c2c1ccc1c2-c2ccccc2C12c1ccccc1-c1ccccc12]

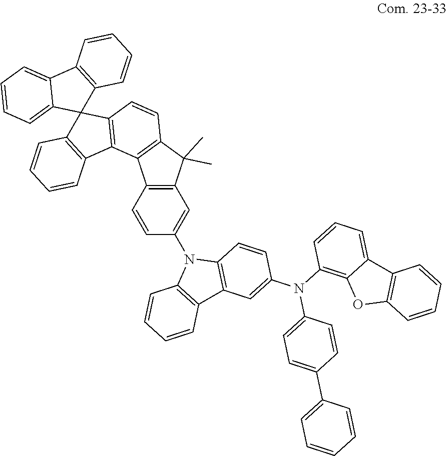 [SMILES: CC1(C)c2cc(-n3c4ccccc4c4cc(N(c5ccc(-c6ccccc6)cc5)c5cccc6c5oc5ccccc56)ccc43)ccc2-c2c1ccc1c2-c2ccccc2C12c1ccccc1-c1ccccc12]

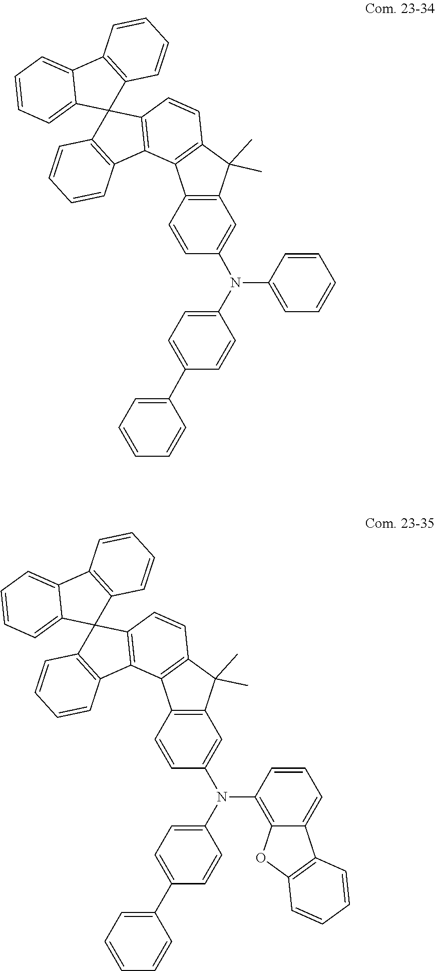 [SMILES: CC1(C)c2cc(N(c3ccccc3)c3ccc(-c4ccccc4)cc3)ccc2-c2c1ccc1c2-c2ccccc2C12c1ccccc1-c1ccccc12 ; CC1(C)c2cc(N(c3ccc(-c4ccccc4)cc3)c3cccc4c3oc3ccccc34)ccc2-c2c1ccc1c2-c2ccccc2C12c1ccccc1-c1ccccc12]

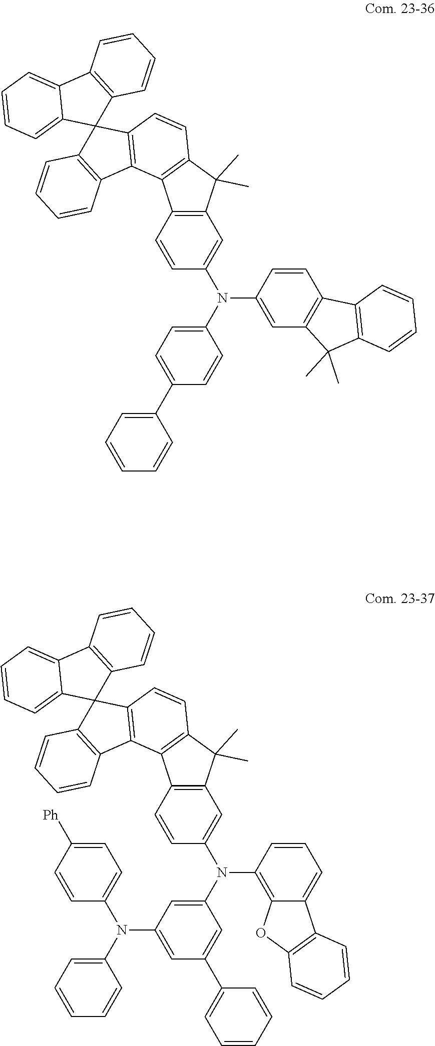 [SMILES: CC1(C)c2ccccc2-c2ccc(N(c3ccc(-c4ccccc4)cc3)c3ccc4c(c3)C(C)(C)c3ccc5c(c3-4)-c3ccccc3C53c4ccccc4-c4ccccc43)cc21 ; CC1(C)c2cc(N(c3cc(-c4ccccc4)cc(N(c4ccccc4)c4ccc(-c5ccccc5)cc4)c3)c3cccc4c3oc3ccccc34)ccc2-c2c1ccc1c2-c2ccccc2C12c1ccccc1-c1ccccc12]

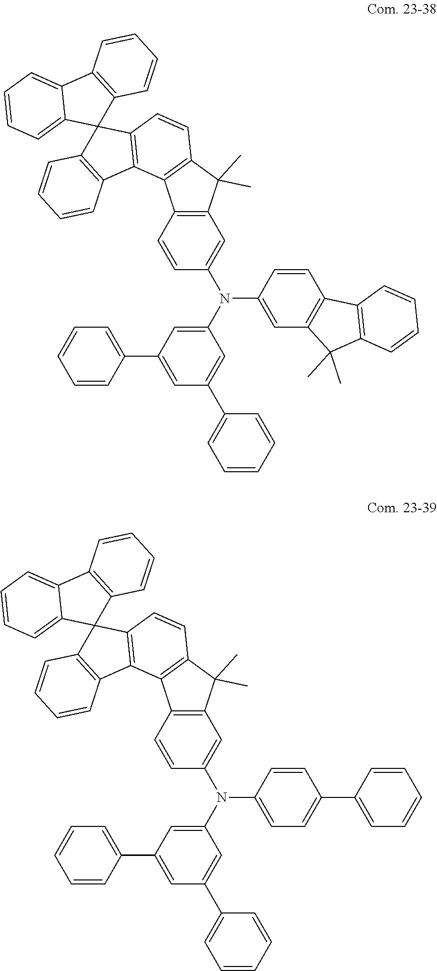 [SMILES: CC1(C)c2ccccc2-c2ccc(N(c3cc(-c4ccccc4)cc(-c4ccccc4)c3)c3ccc4c(c3)C(C)(C)c3ccc5c(c3-4)-c3ccccc3C53c4ccccc4-c4ccccc43)cc21 ; CC1(C)c2cc(N(c3ccc(-c4ccccc4)cc3)c3cc(-c4ccccc4)cc(-c4ccccc4)c3)ccc2-c2c1ccc1c2-c2ccccc2C12c1ccccc1-c1ccccc12]

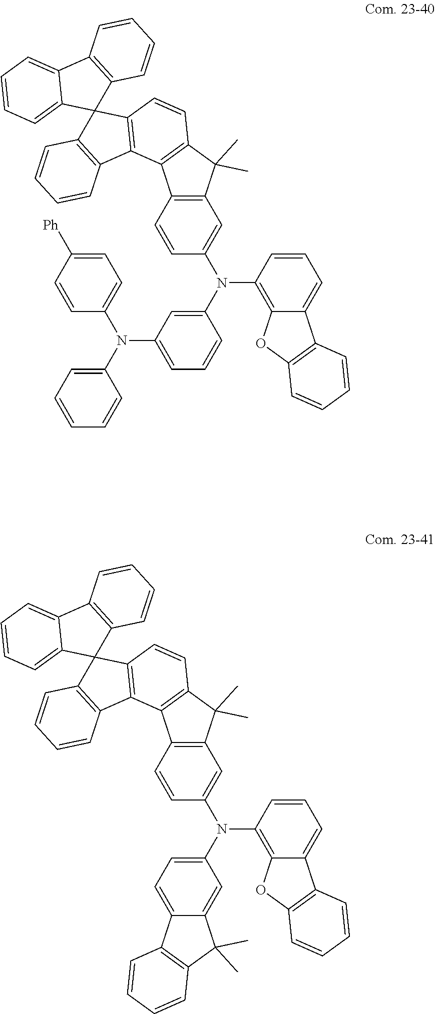 [SMILES: CC1(C)c2cc(N(c3cccc(N(c4ccccc4)c4ccc(-c5ccccc5)cc4)c3)c3cccc4c3oc3ccccc34)ccc2-c2c1ccc1c2-c2ccccc2C12c1ccccc1-c1ccccc12 ; CC1(C)c2ccccc2-c2ccc(N(c3ccc4c(c3)C(C)(C)c3ccc5c(c3-4)-c3ccccc3C53c4ccccc4-c4ccccc43)c3cccc4c3oc3ccccc34)cc21]

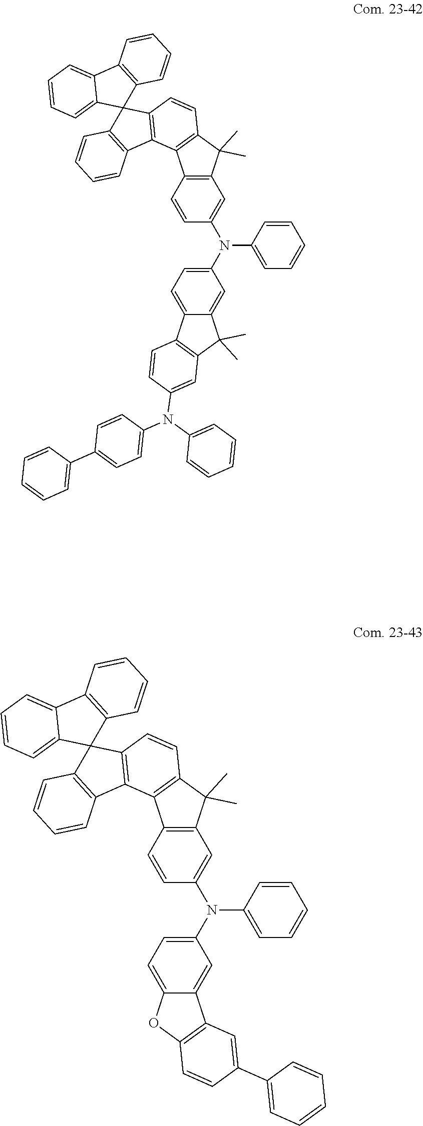 [SMILES: CC1(C)c2cc(N(c3ccccc3)c3ccc(-c4ccccc4)cc3)ccc2-c2ccc(N(c3ccccc3)c3ccc4c(c3)C(C)(C)c3ccc5c(c3-4)-c3ccccc3C53c4ccccc4-c4ccccc43)cc21 ; CC1(C)c2cc(N(c3ccccc3)c3ccc4oc5ccc(-c6ccccc6)cc5c4c3)ccc2-c2c1ccc1c2-c2ccccc2C12c1ccccc1-c1ccccc12]

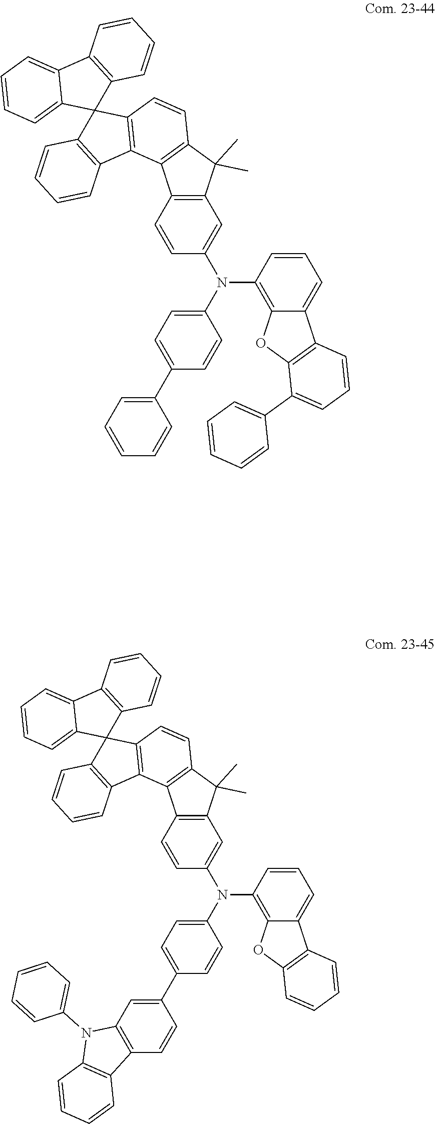 [SMILES: CC1(C)c2cc(N(c3ccc(-c4ccccc4)cc3)c3cccc4c3oc3c(-c5ccccc5)cccc34)ccc2-c2c1ccc1c2-c2ccccc2C12c1ccccc1-c1ccccc12 ; CC1(C)c2cc(N(c3ccc(-c4ccc5c6ccccc6n(-c6ccccc6)c5c4)cc3)c3cccc4c3oc3ccccc34)ccc2-c2c1ccc1c2-c2ccccc2C12c1ccccc1-c1ccccc12]

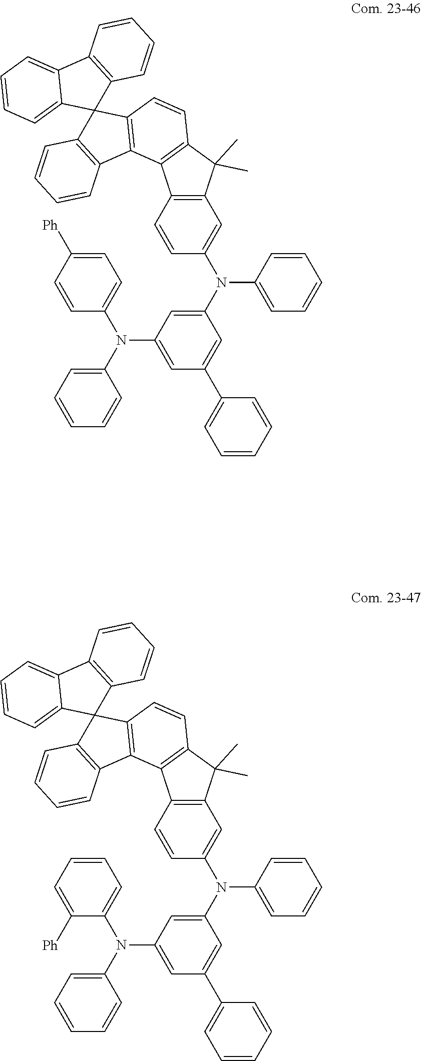 [SMILES: CC1(C)c2cc(N(c3ccccc3)c3cc(-c4ccccc4)cc(N(c4ccccc4)c4ccc(-c5ccccc5)cc4)c3)ccc2-c2c1ccc1c2-c2ccccc2C12c1ccccc1-c1ccccc12 ; CC1(C)c2cc(N(c3ccccc3)c3cc(-c4ccccc4)cc(N(c4ccccc4)c4ccccc4-c4ccccc4)c3)ccc2-c2c1ccc1c2-c2ccccc2C12c1ccccc1-c1ccccc12]

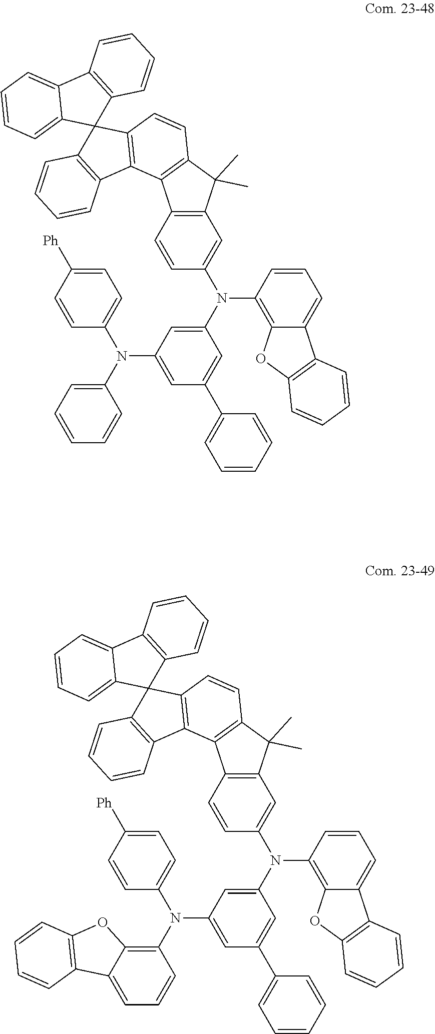 [SMILES: CC1(C)c2cc(N(c3cc(-c4ccccc4)cc(N(c4ccccc4)c4ccc(-c5ccccc5)cc4)c3)c3cccc4c3oc3ccccc34)ccc2-c2c1ccc1c2-c2ccccc2C12c1ccccc1-c1ccccc12 ; CC1(C)c2cc(N(c3cc(-c4ccccc4)cc(N(c4ccc(-c5ccccc5)cc4)c4cccc5c4oc4ccccc45)c3)c3cccc4c3oc3ccccc34)ccc2-c2c1ccc1c2-c2ccccc2C12c1ccccc1-c1ccccc12]

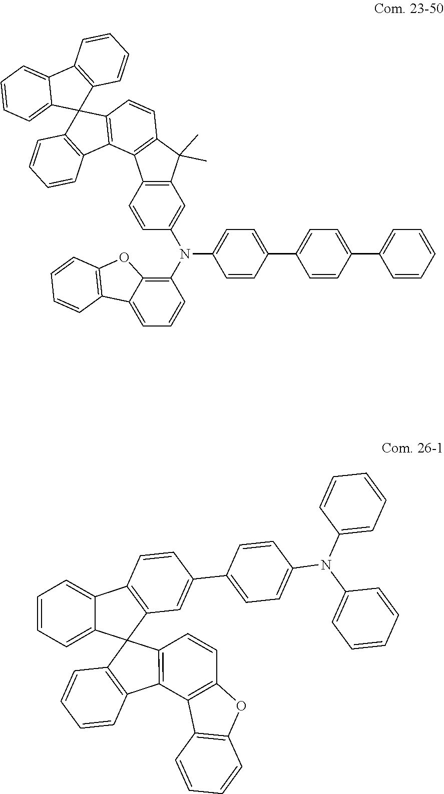 [SMILES: CC1(C)c2cc(N(c3ccc(-c4ccc(-c5ccccc5)cc4)cc3)c3cccc4c3oc3ccccc34)ccc2-c2c1ccc1c2-c2ccccc2C12c1ccccc1-c1ccccc12 ; c1ccc(N(c2ccccc2)c2ccc(-c3ccc4c(c3)C3(c5ccccc5-4)c4ccccc4-c4c3ccc3oc5ccccc5c43)cc2)cc1]

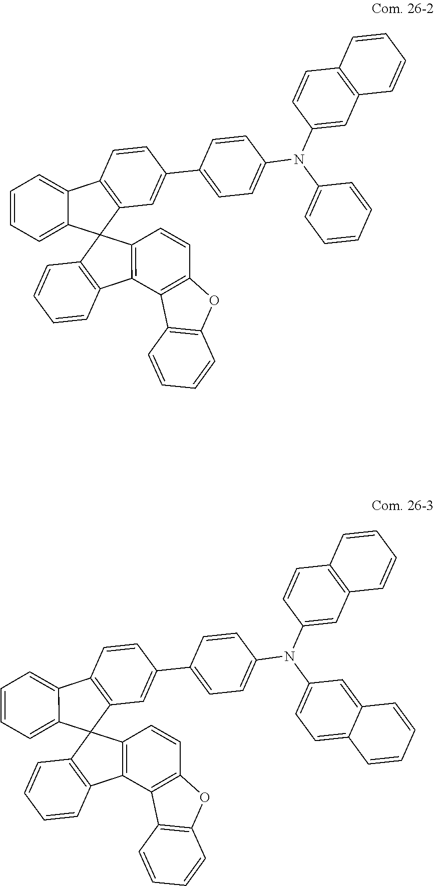 [SMILES: c1ccc(N(c2ccc(-c3ccc4c(c3)C3(c5ccccc5-4)c4ccccc4-c4c3ccc3oc5ccccc5c43)cc2)c2ccc3ccccc3c2)cc1 ; c1ccc2c(c1)-c1ccc(-c3ccc(N(c4ccc5ccccc5c4)c4ccc5ccccc5c4)cc3)cc1C21c2ccccc2-c2c1ccc1oc3ccccc3c21]

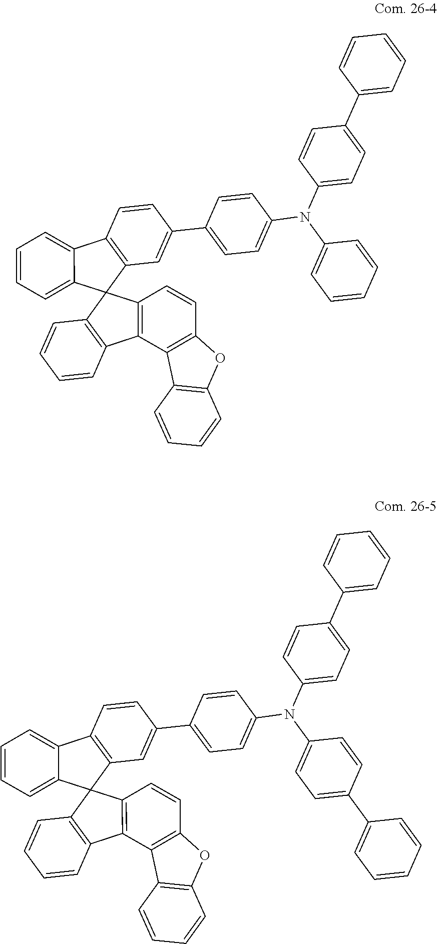 [SMILES: c1ccc(-c2ccc(N(c3ccccc3)c3ccc(-c4ccc5c(c4)C4(c6ccccc6-5)c5ccccc5-c5c4ccc4oc6ccccc6c54)cc3)cc2)cc1 ; c1ccc(-c2ccc(N(c3ccc(-c4ccccc4)cc3)c3ccc(-c4ccc5c(c4)C4(c6ccccc6-5)c5ccccc5-c5c4ccc4oc6ccccc6c54)cc3)cc2)cc1]

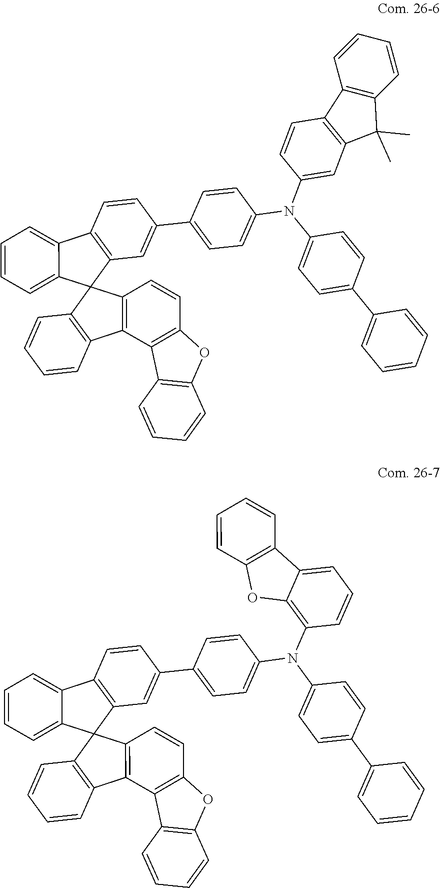 [SMILES: CC1(C)c2ccccc2-c2ccc(N(c3ccc(-c4ccccc4)cc3)c3ccc(-c4ccc5c(c4)C4(c6ccccc6-5)c5ccccc5-c5c4ccc4oc6ccccc6c54)cc3)cc21 ; c1ccc(-c2ccc(N(c3ccc(-c4ccc5c(c4)C4(c6ccccc6-5)c5ccccc5-c5c4ccc4oc6ccccc6c54)cc3)c3cccc4c3oc3ccccc34)cc2)cc1]

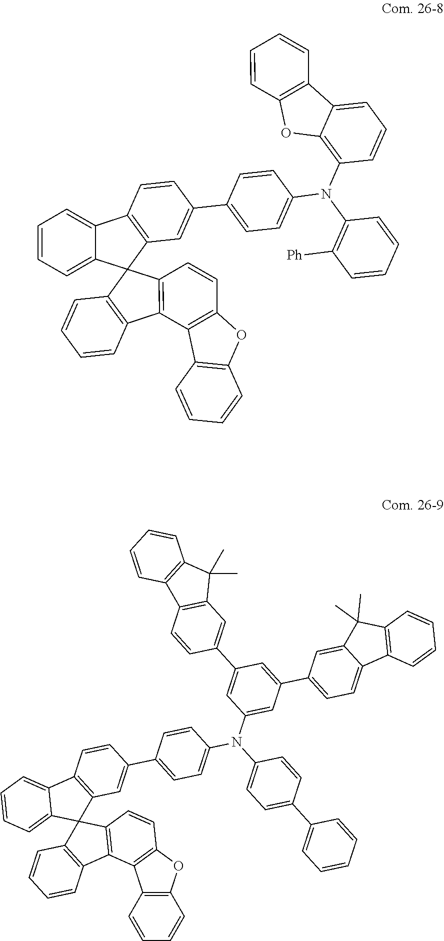 [SMILES: c1ccc(-c2ccccc2N(c2ccc(-c3ccc4c(c3)C3(c5ccccc5-4)c4ccccc4-c4c3ccc3oc5ccccc5c43)cc2)c2cccc3c2oc2ccccc23)cc1 ; CC1(C)c2ccccc2-c2ccc(-c3cc(-c4ccc5c(c4)C(C)(C)c4ccccc4-5)cc(N(c4ccc(-c5ccccc5)cc4)c4ccc(-c5ccc6c(c5)C5(c7ccccc7-6)c6ccccc6-c6c5ccc5oc7ccccc7c65)cc4)c3)cc21]

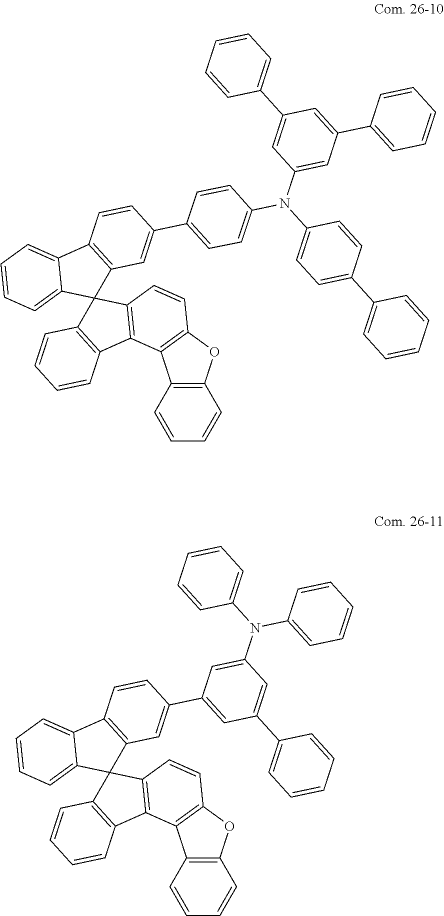 [SMILES: c1ccc(-c2ccc(N(c3ccc(-c4ccc5c(c4)C4(c6ccccc6-5)c5ccccc5-c5c4ccc4oc6ccccc6c54)cc3)c3cc(-c4ccccc4)cc(-c4ccccc4)c3)cc2)cc1 ; c1ccc(-c2cc(-c3ccc4c(c3)C3(c5ccccc5-4)c4ccccc4-c4c3ccc3oc5ccccc5c43)cc(N(c3ccccc3)c3ccccc3)c2)cc1]

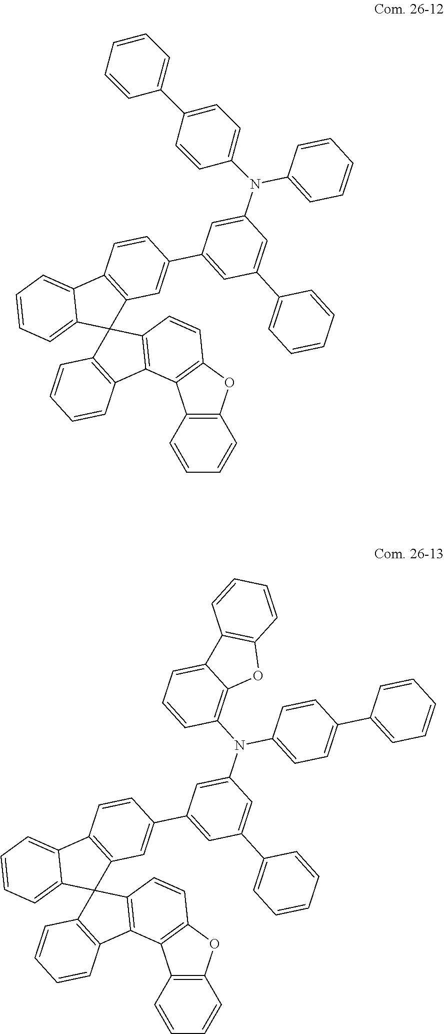 [SMILES: c1ccc(-c2ccc(N(c3ccccc3)c3cc(-c4ccccc4)cc(-c4ccc5c(c4)C4(c6ccccc6-5)c5ccccc5-c5c4ccc4oc6ccccc6c54)c3)cc2)cc1 ; c1ccc(-c2ccc(N(c3cc(-c4ccccc4)cc(-c4ccc5c(c4)C4(c6ccccc6-5)c5ccccc5-c5c4ccc4oc6ccccc6c54)c3)c3cccc4c3oc3ccccc34)cc2)cc1]

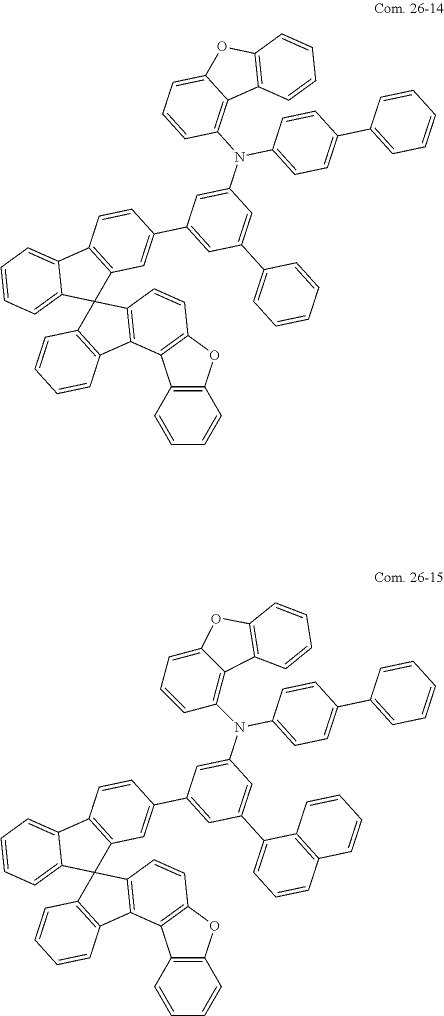 [SMILES: c1ccc(-c2ccc(N(c3cc(-c4ccccc4)cc(-c4ccc5c(c4)C4(c6ccccc6-5)c5ccccc5-c5c4ccc4oc6ccccc6c54)c3)c3cccc4oc5ccccc5c34)cc2)cc1 ; c1ccc(-c2ccc(N(c3cc(-c4ccc5c(c4)C4(c6ccccc6-5)c5ccccc5-c5c4ccc4oc6ccccc6c54)cc(-c4cccc5ccccc45)c3)c3cccc4oc5ccccc5c34)cc2)cc1]

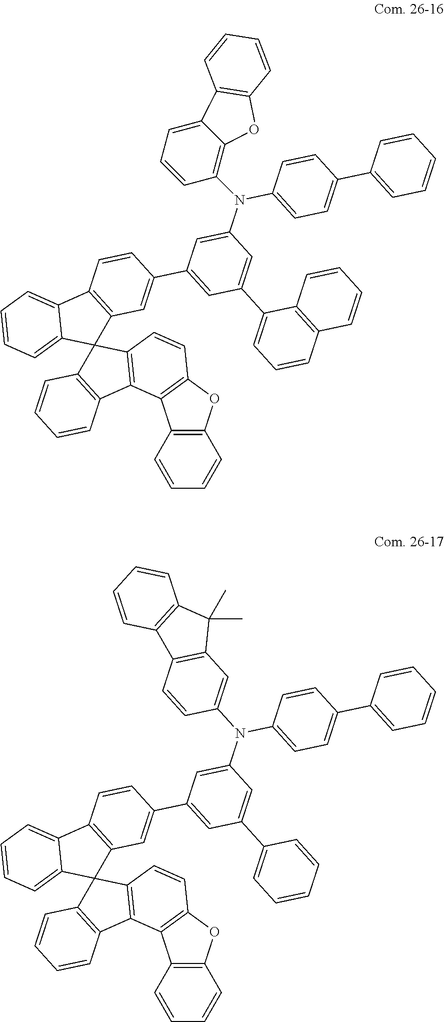 [SMILES: c1ccc(-c2ccc(N(c3cc(-c4ccc5c(c4)C4(c6ccccc6-5)c5ccccc5-c5c4ccc4oc6ccccc6c54)cc(-c4cccc5ccccc45)c3)c3cccc4c3oc3ccccc34)cc2)cc1 ; CC1(C)c2ccccc2-c2ccc(N(c3ccc(-c4ccccc4)cc3)c3cc(-c4ccccc4)cc(-c4ccc5c(c4)C4(c6ccccc6-5)c5ccccc5-c5c4ccc4oc6ccccc6c54)c3)cc21]

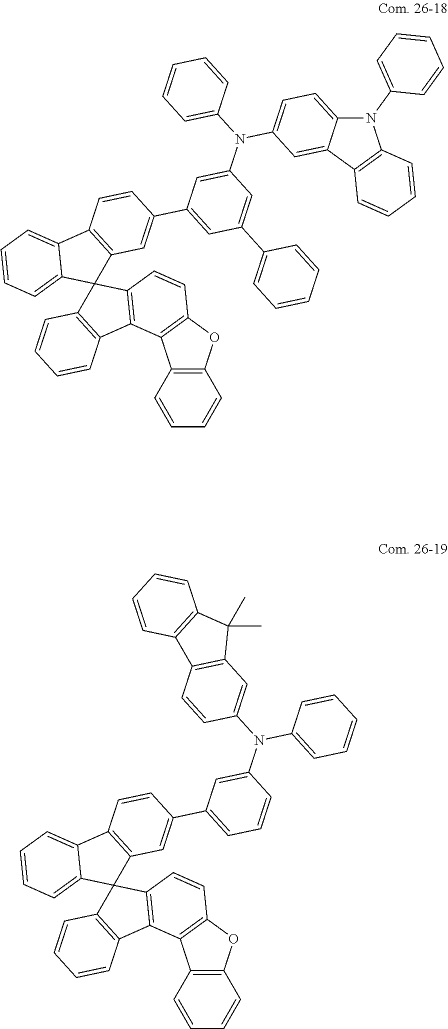 [SMILES: c1ccc(-c2cc(-c3ccc4c(c3)C3(c5ccccc5-4)c4ccccc4-c4c3ccc3oc5ccccc5c43)cc(N(c3ccccc3)c3ccc4c(c3)c3ccccc3n4-c3ccccc3)c2)cc1 ; CC1(C)c2ccccc2-c2ccc(N(c3ccccc3)c3cccc(-c4ccc5c(c4)C4(c6ccccc6-5)c5ccccc5-c5c4ccc4oc6ccccc6c54)c3)cc21]

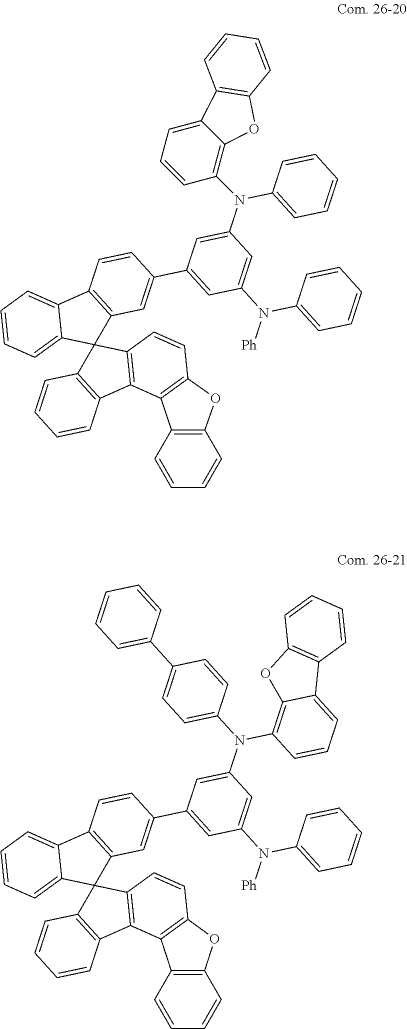 [SMILES: c1ccc(N(c2ccccc2)c2cc(-c3ccc4c(c3)C3(c5ccccc5-4)c4ccccc4-c4c3ccc3oc5ccccc5c43)cc(N(c3ccccc3)c3cccc4c3oc3ccccc34)c2)cc1 ; c1ccc(-c2ccc(N(c3cc(-c4ccc5c(c4)C4(c6ccccc6-5)c5ccccc5-c5c4ccc4oc6ccccc6c54)cc(N(c4ccccc4)c4ccccc4)c3)c3cccc4c3oc3ccccc34)cc2)cc1]

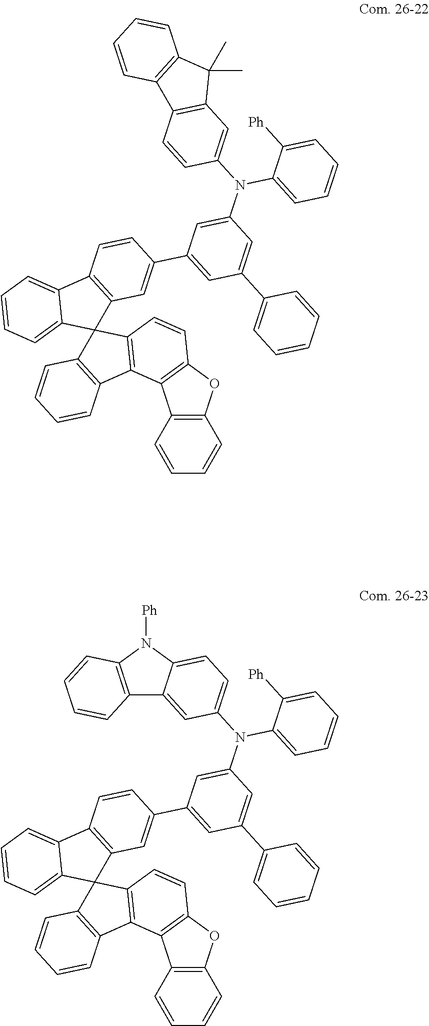 [SMILES: CC1(C)c2ccccc2-c2ccc(N(c3cc(-c4ccccc4)cc(-c4ccc5c(c4)C4(c6ccccc6-5)c5ccccc5-c5c4ccc4oc6ccccc6c54)c3)c3ccccc3-c3ccccc3)cc21 ; c1ccc(-c2cc(-c3ccc4c(c3)C3(c5ccccc5-4)c4ccccc4-c4c3ccc3oc5ccccc5c43)cc(N(c3ccc4c(c3)c3ccccc3n4-c3ccccc3)c3ccccc3-c3ccccc3)c2)cc1]

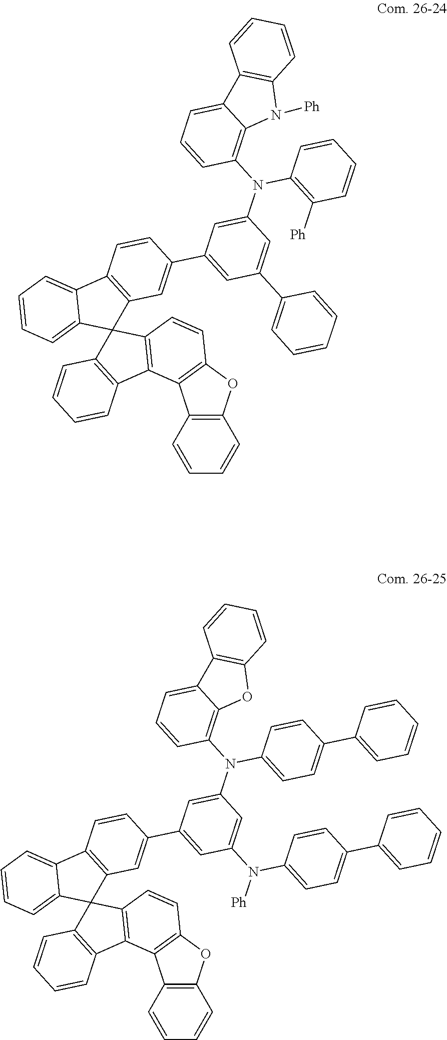 [SMILES: c1ccc(-c2cc(-c3ccc4c(c3)C3(c5ccccc5-4)c4ccccc4-c4c3ccc3oc5ccccc5c43)cc(N(c3ccccc3-c3ccccc3)c3cccc4c5ccccc5n(-c5ccccc5)c34)c2)cc1 ; c1ccc(-c2ccc(N(c3ccccc3)c3cc(-c4ccc5c(c4)C4(c6ccccc6-5)c5ccccc5-c5c4ccc4oc6ccccc6c54)cc(N(c4ccc(-c5ccccc5)cc4)c4cccc5c4oc4ccccc45)c3)cc2)cc1]

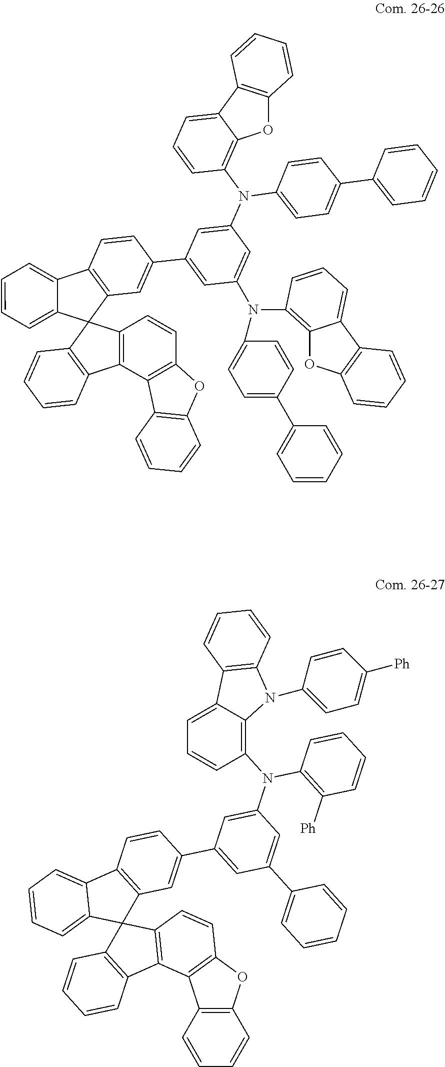 [SMILES: c1ccc(-c2ccc(N(c3cc(-c4ccc5c(c4)C4(c6ccccc6-5)c5ccccc5-c5c4ccc4oc6ccccc6c54)cc(N(c4ccc(-c5ccccc5)cc4)c4cccc5c4oc4ccccc45)c3)c3cccc4c3oc3ccccc34)cc2)cc1 ; c1ccc(-c2ccc(-n3c4ccccc4c4cccc(N(c5cc(-c6ccccc6)cc(-c6ccc7c(c6)C6(c8ccccc8-7)c7ccccc7-c7c6ccc6oc8ccccc8c76)c5)c5ccccc5-c5ccccc5)c43)cc2)cc1]

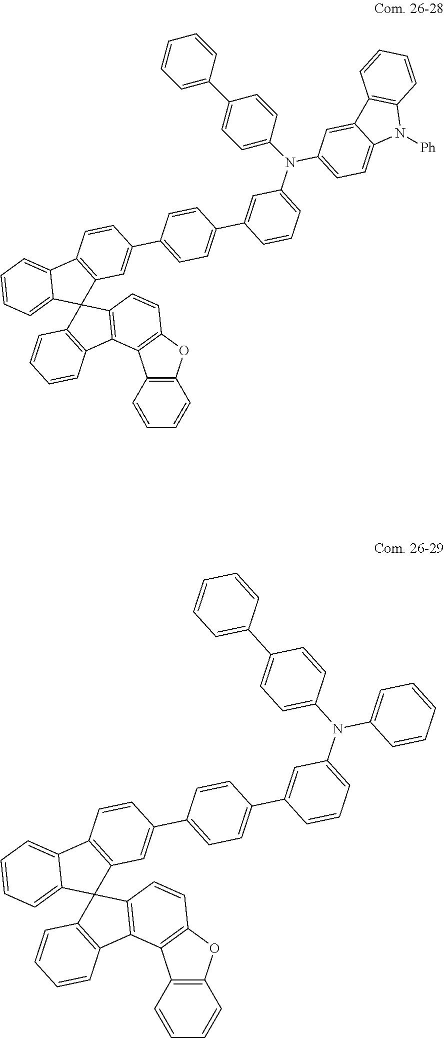 [SMILES: c1ccc(-c2ccc(N(c3cccc(-c4ccc(-c5ccc6c(c5)C5(c7ccccc7-6)c6ccccc6-c6c5ccc5oc7ccccc7c65)cc4)c3)c3ccc4c(c3)c3ccccc3n4-c3ccccc3)cc2)cc1 ; c1ccc(-c2ccc(N(c3ccccc3)c3cccc(-c4ccc(-c5ccc6c(c5)C5(c7ccccc7-6)c6ccccc6-c6c5ccc5oc7ccccc7c65)cc4)c3)cc2)cc1]

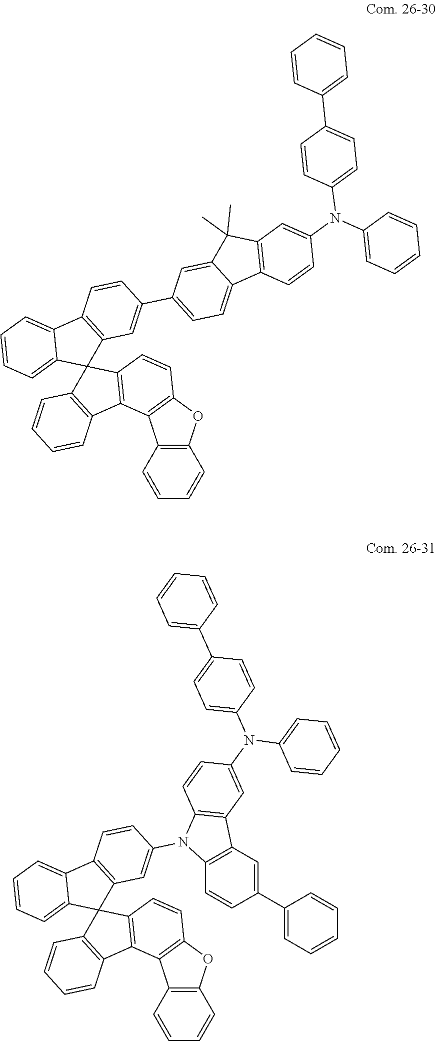 [SMILES: CC1(C)c2cc(-c3ccc4c(c3)C3(c5ccccc5-4)c4ccccc4-c4c3ccc3oc5ccccc5c43)ccc2-c2ccc(N(c3ccccc3)c3ccc(-c4ccccc4)cc3)cc21 ; c1ccc(-c2ccc(N(c3ccccc3)c3ccc4c(c3)c3cc(-c5ccccc5)ccc3n4-c3ccc4c(c3)C3(c5ccccc5-4)c4ccccc4-c4c3ccc3oc5ccccc5c43)cc2)cc1]

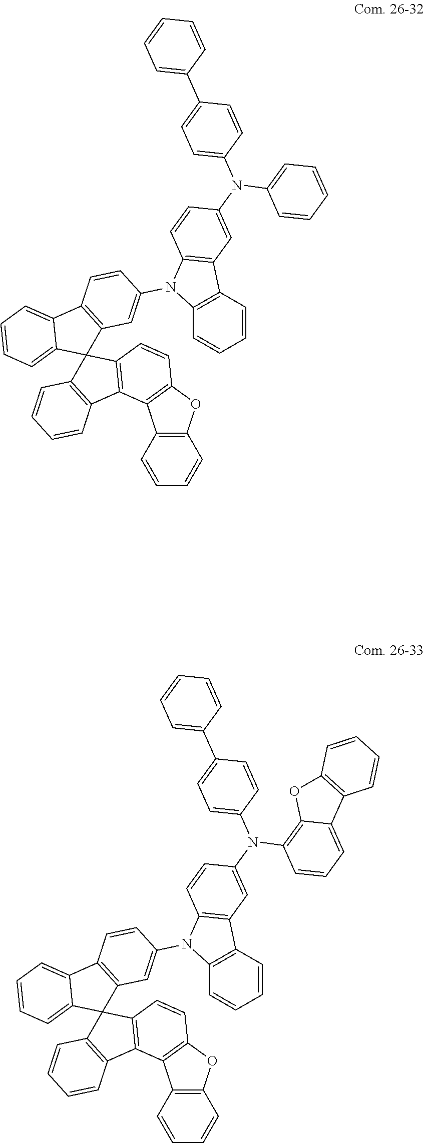 [SMILES: c1ccc(-c2ccc(N(c3ccccc3)c3ccc4c(c3)c3ccccc3n4-c3ccc4c(c3)C3(c5ccccc5-4)c4ccccc4-c4c3ccc3oc5ccccc5c43)cc2)cc1 ; c1ccc(-c2ccc(N(c3ccc4c(c3)c3ccccc3n4-c3ccc4c(c3)C3(c5ccccc5-4)c4ccccc4-c4c3ccc3oc5ccccc5c43)c3cccc4c3oc3ccccc34)cc2)cc1]

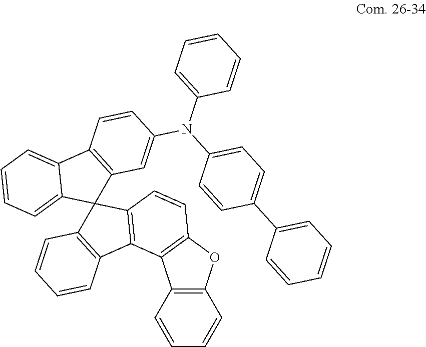 [SMILES: c1ccc(-c2ccc(N(c3ccccc3)c3ccc4c(c3)C3(c5ccccc5-4)c4ccccc4-c4c3ccc3oc5ccccc5c43)cc2)cc1]

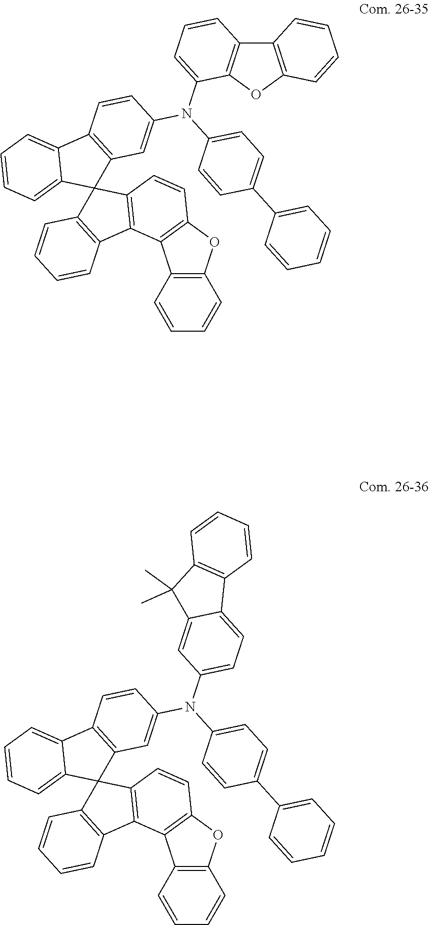 [SMILES: c1ccc(-c2ccc(N(c3ccc4c(c3)C3(c5ccccc5-4)c4ccccc4-c4c3ccc3oc5ccccc5c43)c3cccc4c3oc3ccccc34)cc2)cc1 ; CC1(C)c2ccccc2-c2ccc(N(c3ccc(-c4ccccc4)cc3)c3ccc4c(c3)C3(c5ccccc5-4)c4ccccc4-c4c3ccc3oc5ccccc5c43)cc21]

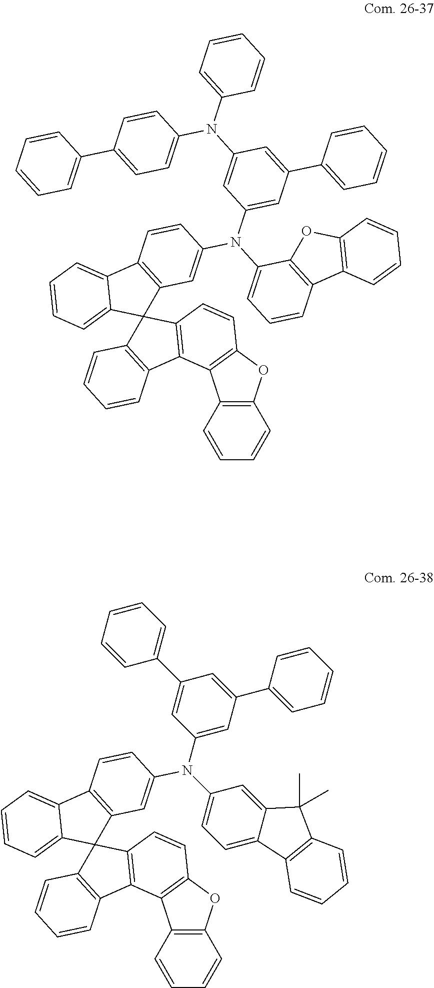 [SMILES: c1ccc(-c2ccc(N(c3ccccc3)c3cc(-c4ccccc4)cc(N(c4ccc5c(c4)C4(c6ccccc6-5)c5ccccc5-c5c4ccc4oc6ccccc6c54)c4cccc5c4oc4ccccc45)c3)cc2)cc1 ; CC1(C)c2ccccc2-c2ccc(N(c3cc(-c4ccccc4)cc(-c4ccccc4)c3)c3ccc4c(c3)C3(c5ccccc5-4)c4ccccc4-c4c3ccc3oc5ccccc5c43)cc21]

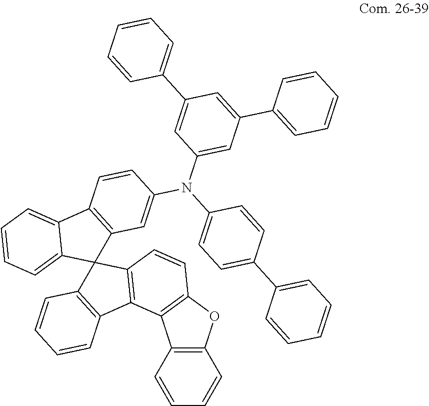 [SMILES: c1ccc(-c2ccc(N(c3cc(-c4ccccc4)cc(-c4ccccc4)c3)c3ccc4c(c3)C3(c5ccccc5-4)c4ccccc4-c4c3ccc3oc5ccccc5c43)cc2)cc1]

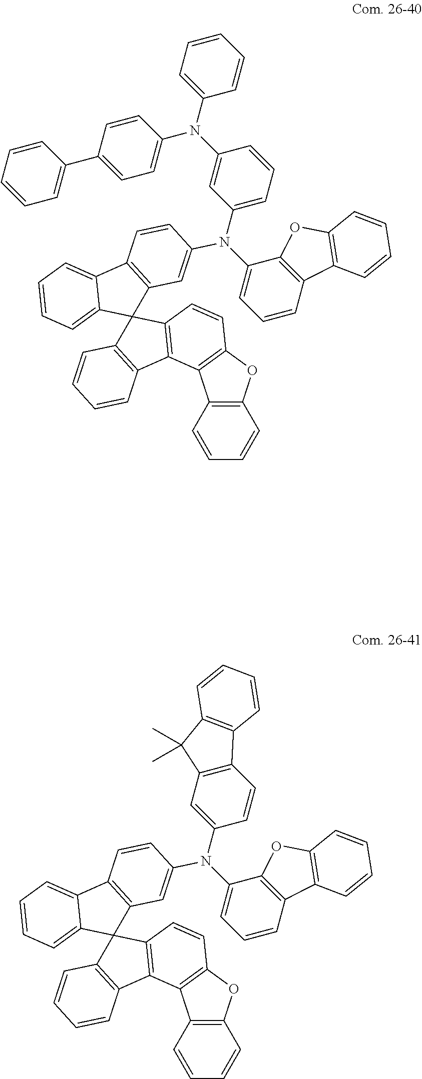 [SMILES: c1ccc(-c2ccc(N(c3ccccc3)c3cccc(N(c4ccc5c(c4)C4(c6ccccc6-5)c5ccccc5-c5c4ccc4oc6ccccc6c54)c4cccc5c4oc4ccccc45)c3)cc2)cc1 ; CC1(C)c2ccccc2-c2ccc(N(c3ccc4c(c3)C3(c5ccccc5-4)c4ccccc4-c4c3ccc3oc5ccccc5c43)c3cccc4c3oc3ccccc34)cc21]

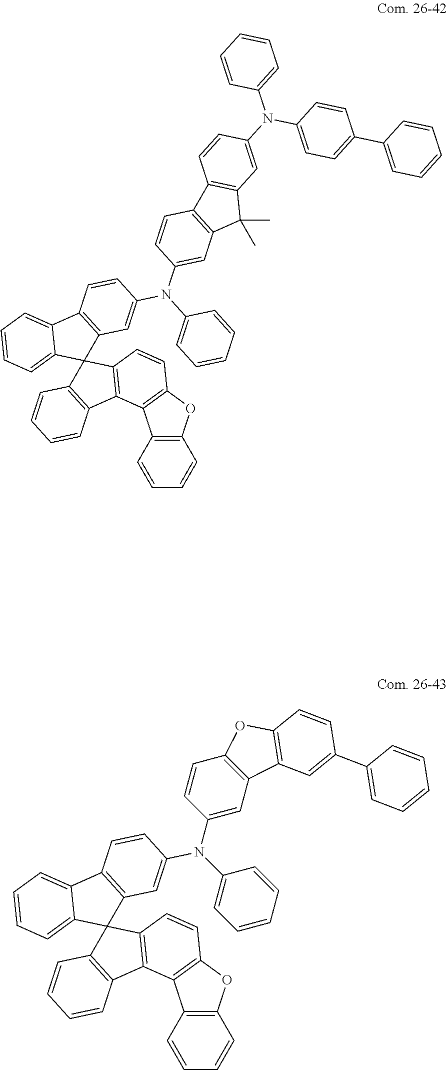 [SMILES: CC1(C)c2cc(N(c3ccccc3)c3ccc(-c4ccccc4)cc3)ccc2-c2ccc(N(c3ccccc3)c3ccc4c(c3)C3(c5ccccc5-4)c4ccccc4-c4c3ccc3oc5ccccc5c43)cc21 ; c1ccc(-c2ccc3oc4ccc(N(c5ccccc5)c5ccc6c(c5)C5(c7ccccc7-6)c6ccccc6-c6c5ccc5oc7ccccc7c65)cc4c3c2)cc1]

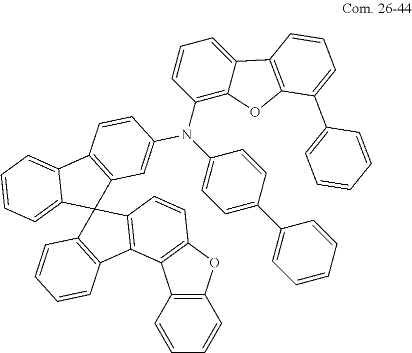 [SMILES: c1ccc(-c2ccc(N(c3ccc4c(c3)C3(c5ccccc5-4)c4ccccc4-c4c3ccc3oc5ccccc5c43)c3cccc4c3oc3c(-c5ccccc5)cccc34)cc2)cc1]

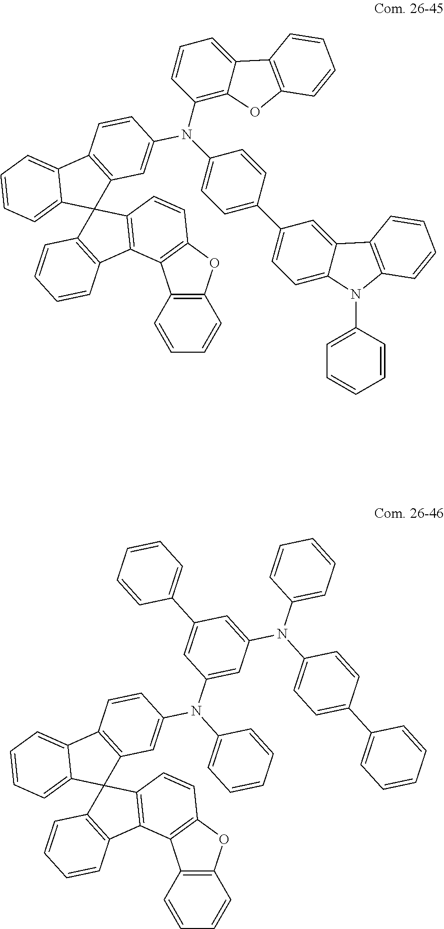 [SMILES: c1ccc(-n2c3ccccc3c3cc(-c4ccc(N(c5ccc6c(c5)C5(c7ccccc7-6)c6ccccc6-c6c5ccc5oc7ccccc7c65)c5cccc6c5oc5ccccc56)cc4)ccc32)cc1 ; c1ccc(-c2ccc(N(c3ccccc3)c3cc(-c4ccccc4)cc(N(c4ccccc4)c4ccc5c(c4)C4(c6ccccc6-5)c5ccccc5-c5c4ccc4oc6ccccc6c54)c3)cc2)cc1]

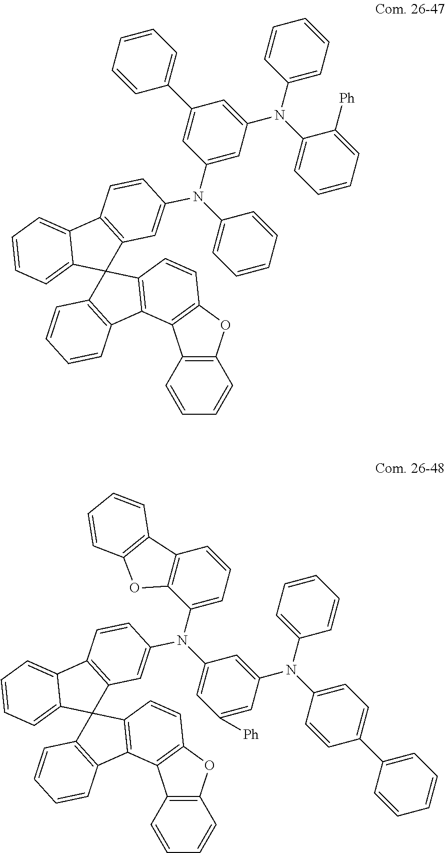 [SMILES: c1ccc(-c2cc(N(c3ccccc3)c3ccc4c(c3)C3(c5ccccc5-4)c4ccccc4-c4c3ccc3oc5ccccc5c43)cc(N(c3ccccc3)c3ccccc3-c3ccccc3)c2)cc1 ; c1ccc(-c2ccc(N(c3ccccc3)c3cc(-c4ccccc4)cc(N(c4ccc5c(c4)C4(c6ccccc6-5)c5ccccc5-c5c4ccc4oc6ccccc6c54)c4cccc5c4oc4ccccc45)c3)cc2)cc1]

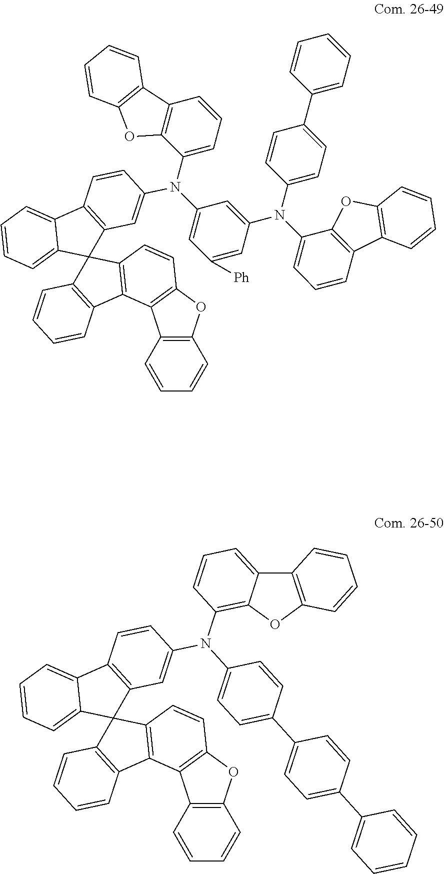 [SMILES: c1ccc(-c2ccc(N(c3cc(-c4ccccc4)cc(N(c4ccc5c(c4)C4(c6ccccc6-5)c5ccccc5-c5c4ccc4oc6ccccc6c54)c4cccc5c4oc4ccccc45)c3)c3cccc4c3oc3ccccc34)cc2)cc1 ; c1ccc(-c2ccc(-c3ccc(N(c4ccc5c(c4)C4(c6ccccc6-5)c5ccccc5-c5c4ccc4oc6ccccc6c54)c4cccc5c4oc4ccccc45)cc3)cc2)cc1]

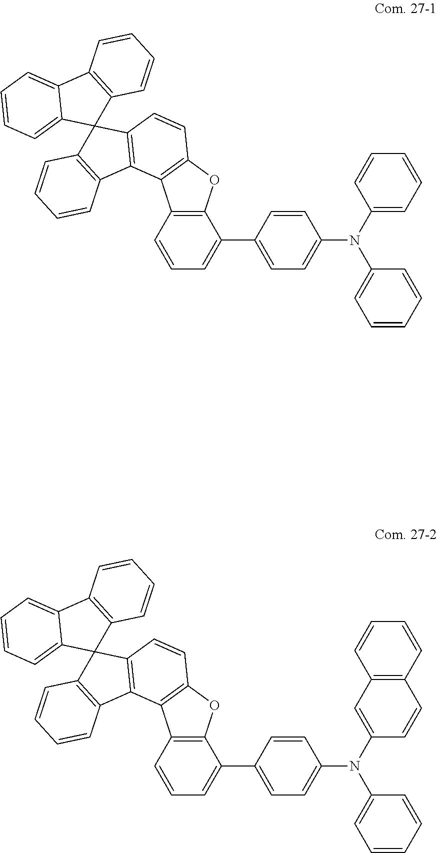 [SMILES: c1ccc(N(c2ccccc2)c2ccc(-c3cccc4c3oc3ccc5c(c34)-c3ccccc3C53c4ccccc4-c4ccccc43)cc2)cc1 ; c1ccc(N(c2ccc(-c3cccc4c3oc3ccc5c(c34)-c3ccccc3C53c4ccccc4-c4ccccc43)cc2)c2ccc3ccccc3c2)cc1]

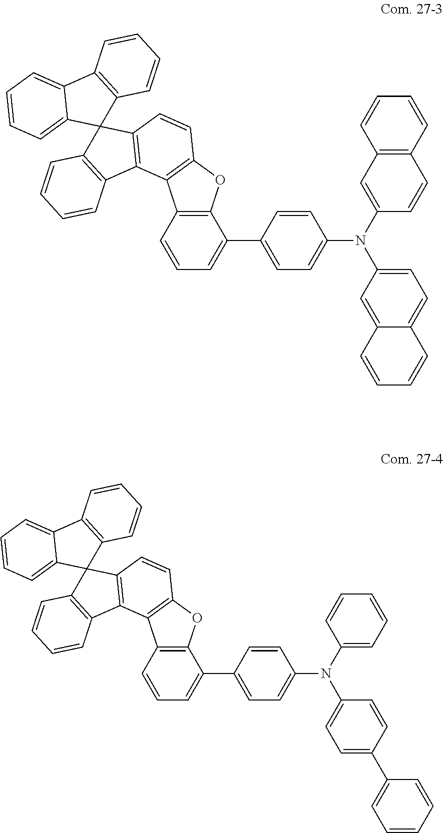 [SMILES: c1ccc2c(c1)-c1ccccc1C21c2ccccc2-c2c1ccc1oc3c(-c4ccc(N(c5ccc6ccccc6c5)c5ccc6ccccc6c5)cc4)cccc3c21 ; c1ccc(-c2ccc(N(c3ccccc3)c3ccc(-c4cccc5c4oc4ccc6c(c45)-c4ccccc4C64c5ccccc5-c5ccccc54)cc3)cc2)cc1]

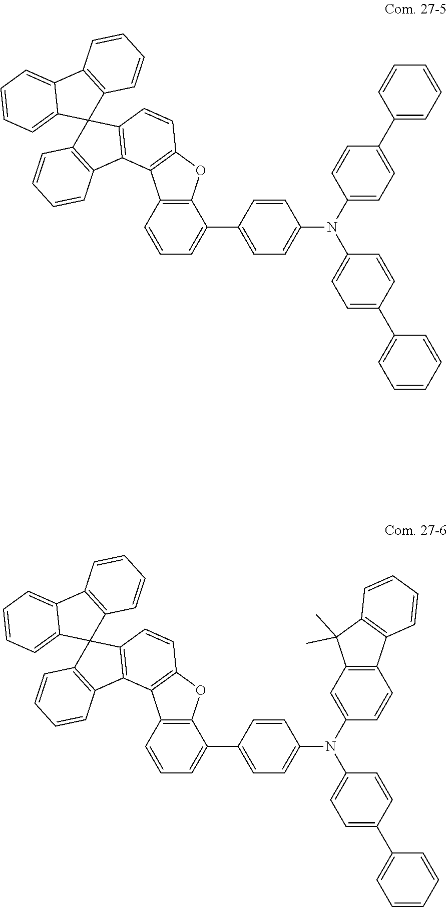 [SMILES: c1ccc(-c2ccc(N(c3ccc(-c4ccccc4)cc3)c3ccc(-c4cccc5c4oc4ccc6c(c45)-c4ccccc4C64c5ccccc5-c5ccccc54)cc3)cc2)cc1 ; CC1(C)c2ccccc2-c2ccc(N(c3ccc(-c4ccccc4)cc3)c3ccc(-c4cccc5c4oc4ccc6c(c45)-c4ccccc4C64c5ccccc5-c5ccccc54)cc3)cc21]

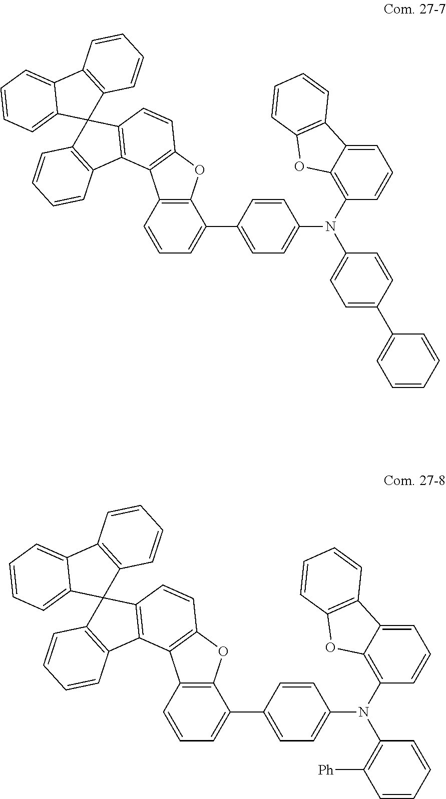 [SMILES: c1ccc(-c2ccc(N(c3ccc(-c4cccc5c4oc4ccc6c(c45)-c4ccccc4C64c5ccccc5-c5ccccc54)cc3)c3cccc4c3oc3ccccc34)cc2)cc1 ; c1ccc(-c2ccccc2N(c2ccc(-c3cccc4c3oc3ccc5c(c34)-c3ccccc3C53c4ccccc4-c4ccccc43)cc2)c2cccc3c2oc2ccccc23)cc1]

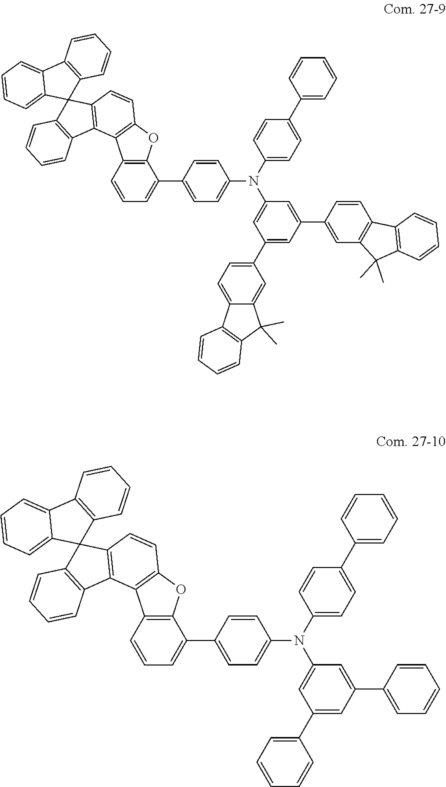 [SMILES: CC1(C)c2ccccc2-c2ccc(-c3cc(-c4ccc5c(c4)C(C)(C)c4ccccc4-5)cc(N(c4ccc(-c5ccccc5)cc4)c4ccc(-c5cccc6c5oc5ccc7c(c56)-c5ccccc5C75c6ccccc6-c6ccccc65)cc4)c3)cc21 ; c1ccc(-c2ccc(N(c3ccc(-c4cccc5c4oc4ccc6c(c45)-c4ccccc4C64c5ccccc5-c5ccccc54)cc3)c3cc(-c4ccccc4)cc(-c4ccccc4)c3)cc2)cc1]

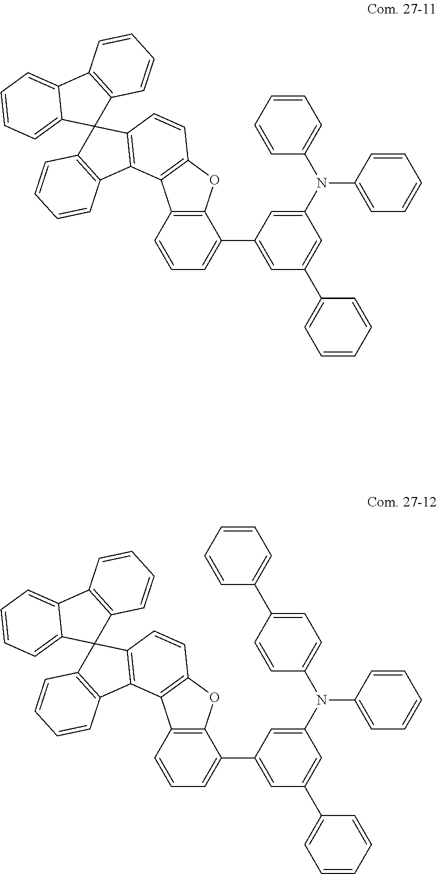 [SMILES: c1ccc(-c2cc(-c3cccc4c3oc3ccc5c(c34)-c3ccccc3C53c4ccccc4-c4ccccc43)cc(N(c3ccccc3)c3ccccc3)c2)cc1 ; c1ccc(-c2ccc(N(c3ccccc3)c3cc(-c4ccccc4)cc(-c4cccc5c4oc4ccc6c(c45)-c4ccccc4C64c5ccccc5-c5ccccc54)c3)cc2)cc1]

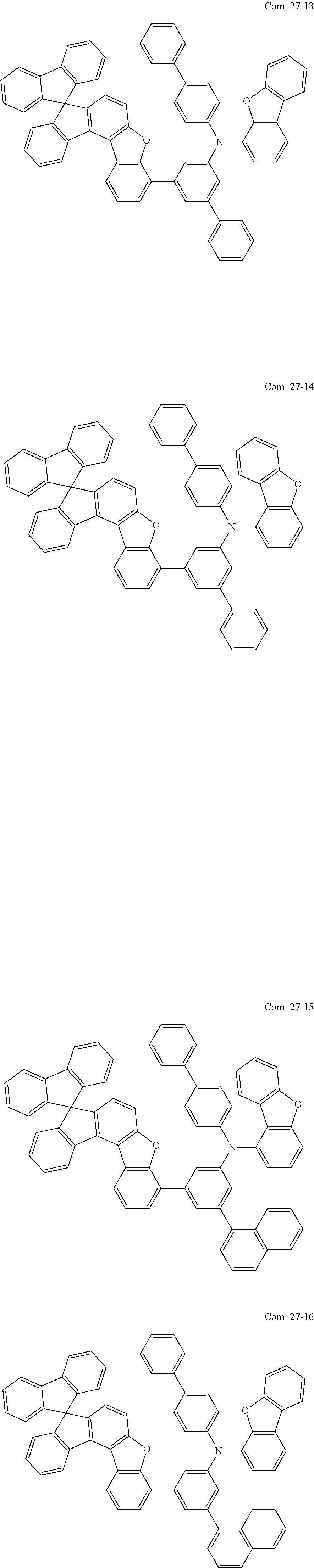 [SMILES: c1ccc(-c2ccc(N(c3cc(-c4ccccc4)cc(-c4cccc5c4oc4ccc6c(c45)-c4ccccc4C64c5ccccc5-c5ccccc54)c3)c3cccc4c3oc3ccccc34)cc2)cc1 ; c1ccc(-c2ccc(N(c3cc(-c4ccccc4)cc(-c4cccc5c4oc4ccc6c(c45)-c4ccccc4C64c5ccccc5-c5ccccc54)c3)c3cccc4oc5ccccc5c34)cc2)cc1 ; c1ccc(-c2ccc(N(c3cc(-c4cccc5ccccc45)cc(-c4cccc5c4oc4ccc6c(c45)-c4ccccc4C64c5ccccc5-c5ccccc54)c3)c3cccc4oc5ccccc5c34)cc2)cc1 ; c1ccc(-c2ccc(N(c3cc(-c4cccc5ccccc45)cc(-c4cccc5c4oc4ccc6c(c45)-c4ccccc4C64c5ccccc5-c5ccccc54)c3)c3cccc4c3oc3ccccc34)cc2)cc1]

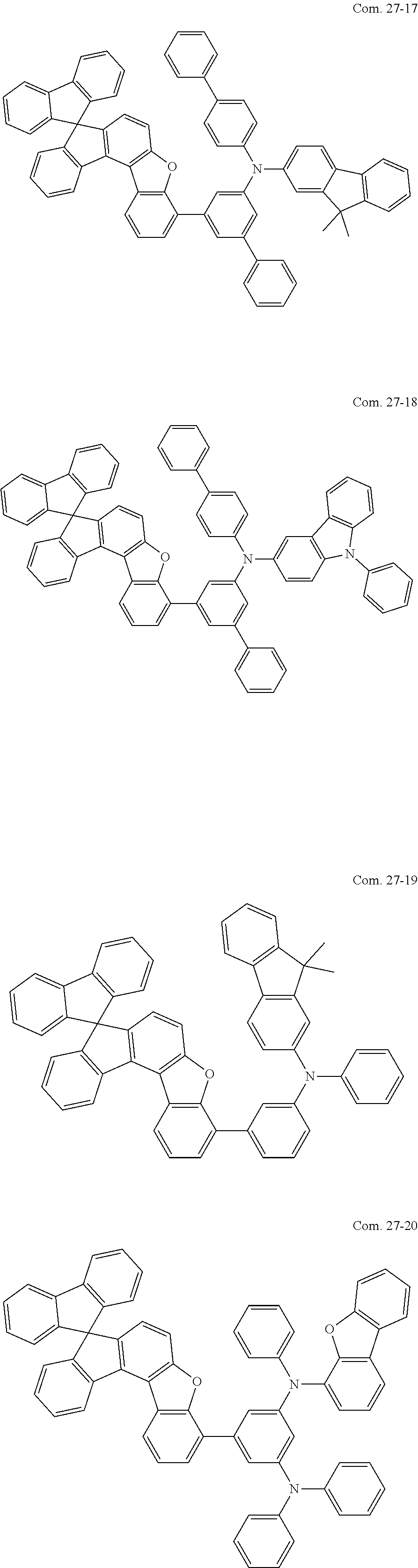 [SMILES: CC1(C)c2ccccc2-c2ccc(N(c3ccc(-c4ccccc4)cc3)c3cc(-c4ccccc4)cc(-c4cccc5c4oc4ccc6c(c45)-c4ccccc4C64c5ccccc5-c5ccccc54)c3)cc21 ; c1ccc(-c2ccc(N(c3cc(-c4ccccc4)cc(-c4cccc5c4oc4ccc6c(c45)-c4ccccc4C64c5ccccc5-c5ccccc54)c3)c3ccc4c(c3)c3ccccc3n4-c3ccccc3)cc2)cc1 ; CC1(C)c2ccccc2-c2ccc(N(c3ccccc3)c3cccc(-c4cccc5c4oc4ccc6c(c45)-c4ccccc4C64c5ccccc5-c5ccccc54)c3)cc21 ; c1ccc(N(c2ccccc2)c2cc(-c3cccc4c3oc3ccc5c(c34)-c3ccccc3C53c4ccccc4-c4ccccc43)cc(N(c3ccccc3)c3cccc4c3oc3ccccc34)c2)cc1]

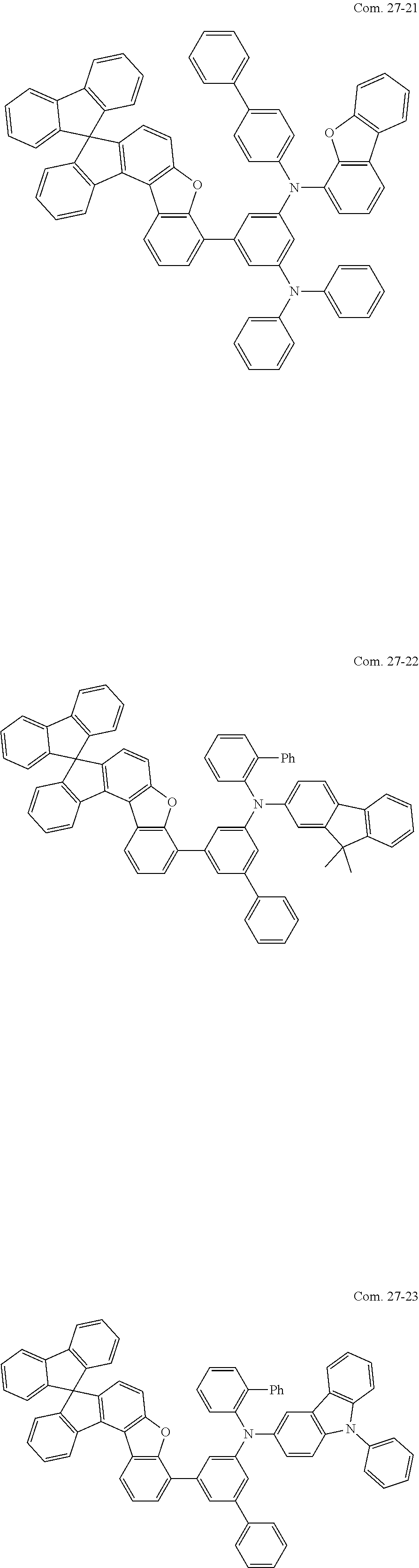 [SMILES: c1ccc(-c2ccc(N(c3cc(-c4cccc5c4oc4ccc6c(c45)-c4ccccc4C64c5ccccc5-c5ccccc54)cc(N(c4ccccc4)c4ccccc4)c3)c3cccc4c3oc3ccccc34)cc2)cc1 ; CC1(C)c2ccccc2-c2ccc(N(c3cc(-c4ccccc4)cc(-c4cccc5c4oc4ccc6c(c45)-c4ccccc4C64c5ccccc5-c5ccccc54)c3)c3ccccc3-c3ccccc3)cc21 ; c1ccc(-c2cc(-c3cccc4c3oc3ccc5c(c34)-c3ccccc3C53c4ccccc4-c4ccccc43)cc(N(c3ccc4c(c3)c3ccccc3n4-c3ccccc3)c3ccccc3-c3ccccc3)c2)cc1]

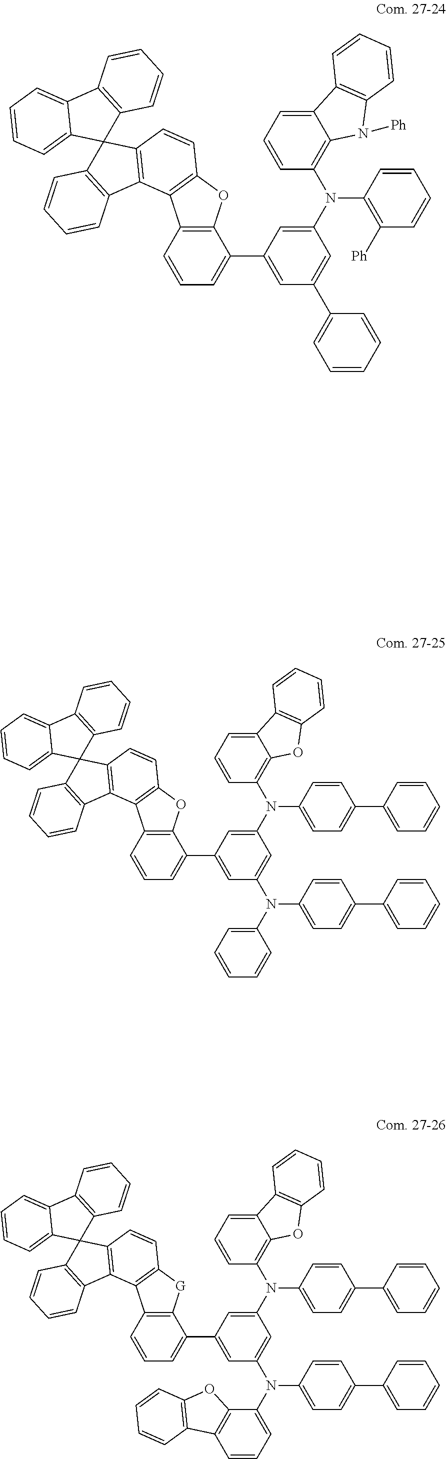

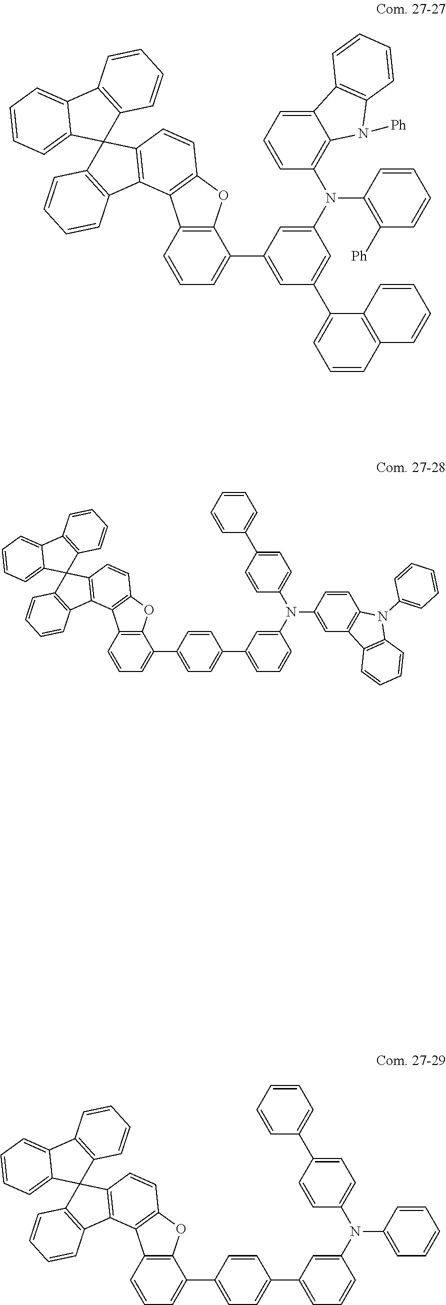 [SMILES: c1ccc(-c2ccccc2N(c2cc(-c3cccc4ccccc34)cc(-c3cccc4c3oc3ccc5c(c34)-c3ccccc3C53c4ccccc4-c4ccccc43)c2)c2cccc3c4ccccc4n(-c4ccccc4)c23)cc1 ; c1ccc(-c2ccc(N(c3cccc(-c4ccc(-c5cccc6c5oc5ccc7c(c56)-c5ccccc5C75c6ccccc6-c6ccccc65)cc4)c3)c3ccc4c(c3)c3ccccc3n4-c3ccccc3)cc2)cc1 ; c1ccc(-c2ccc(N(c3ccccc3)c3cccc(-c4ccc(-c5cccc6c5oc5ccc7c(c56)-c5ccccc5C75c6ccccc6-c6ccccc65)cc4)c3)cc2)cc1]

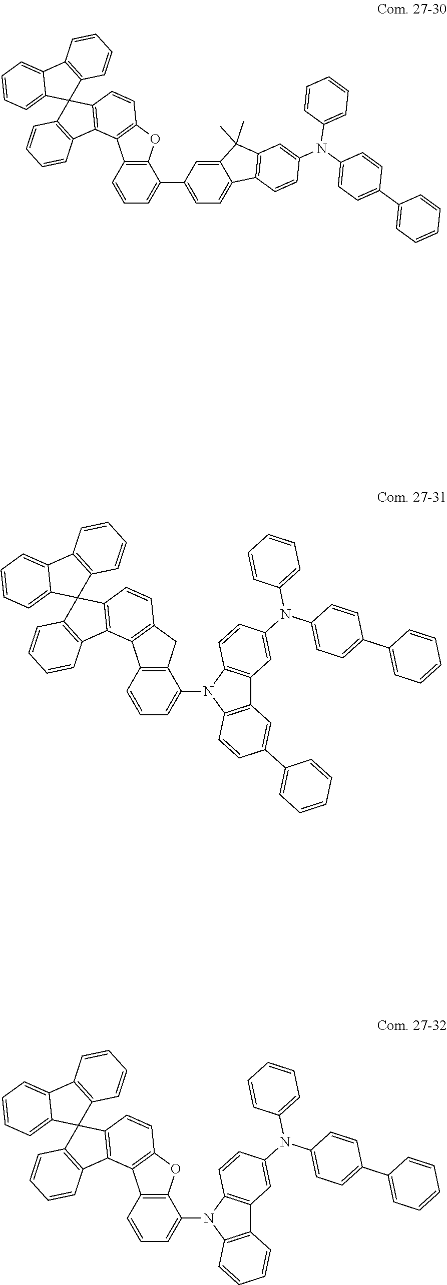 [SMILES: CC1(C)c2cc(-c3cccc4c3oc3ccc5c(c34)-c3ccccc3C53c4ccccc4-c4ccccc43)ccc2-c2ccc(N(c3ccccc3)c3ccc(-c4ccccc4)cc3)cc21 ; c1ccc(-c2ccc(N(c3ccccc3)c3ccc4c(c3)c3cc(-c5ccccc5)ccc3n4-c3cccc4c3Cc3ccc5c(c3-4)-c3ccccc3C53c4ccccc4-c4ccccc43)cc2)cc1 ; c1ccc(-c2ccc(N(c3ccccc3)c3ccc4c(c3)c3ccccc3n4-c3cccc4c3oc3ccc5c(c34)-c3ccccc3C53c4ccccc4-c4ccccc43)cc2)cc1]

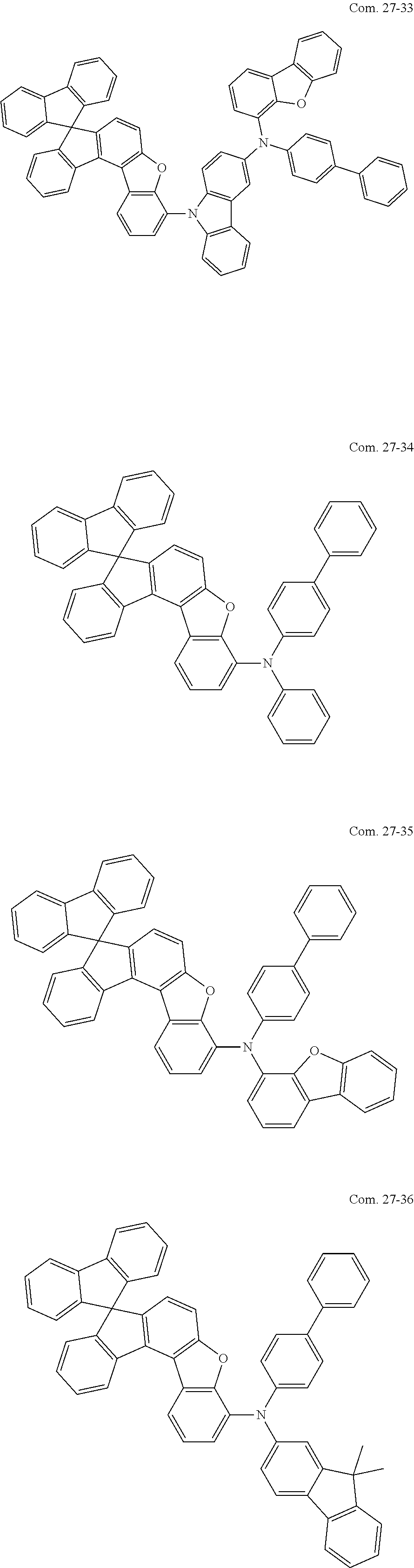 [SMILES: c1ccc(-c2ccc(N(c3ccc4c(c3)c3ccccc3n4-c3cccc4c3oc3ccc5c(c34)-c3ccccc3C53c4ccccc4-c4ccccc43)c3cccc4c3oc3ccccc34)cc2)cc1 ; c1ccc(-c2ccc(N(c3ccccc3)c3cccc4c3oc3ccc5c(c34)-c3ccccc3C53c4ccccc4-c4ccccc43)cc2)cc1 ; c1ccc(-c2ccc(N(c3cccc4c3oc3ccccc34)c3cccc4c3oc3ccc5c(c34)-c3ccccc3C53c4ccccc4-c4ccccc43)cc2)cc1 ; CC1(C)c2ccccc2-c2ccc(N(c3ccc(-c4ccccc4)cc3)c3cccc4c3oc3ccc5c(c34)-c3ccccc3C53c4ccccc4-c4ccccc43)cc21]

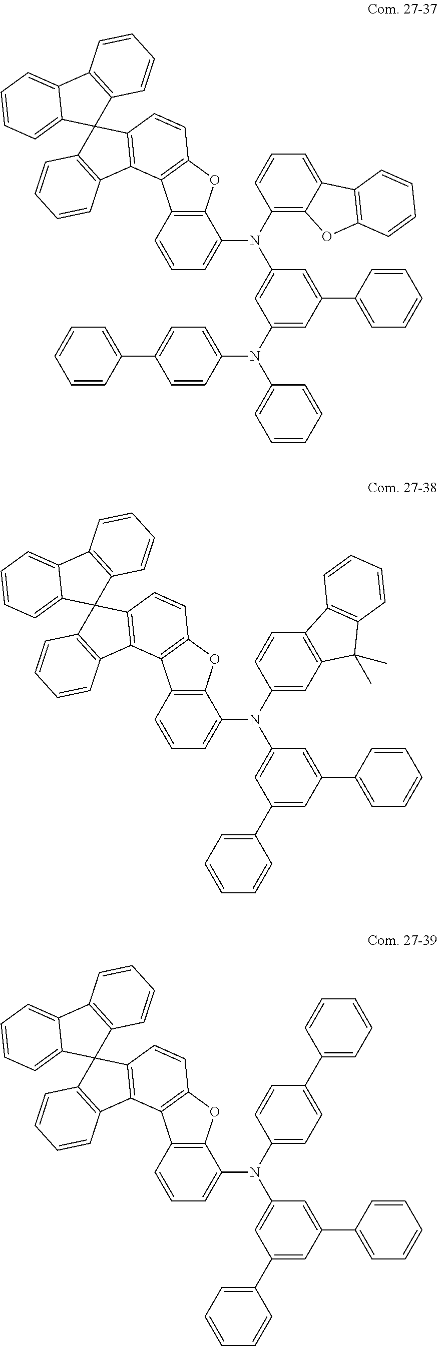 [SMILES: c1ccc(-c2ccc(N(c3ccccc3)c3cc(-c4ccccc4)cc(N(c4cccc5c4oc4ccccc45)c4cccc5c4oc4ccc6c(c45)-c4ccccc4C64c5ccccc5-c5ccccc54)c3)cc2)cc1 ; CC1(C)c2ccccc2-c2ccc(N(c3cc(-c4ccccc4)cc(-c4ccccc4)c3)c3cccc4c3oc3ccc5c(c34)-c3ccccc3C53c4ccccc4-c4ccccc43)cc21 ; c1ccc(-c2ccc(N(c3cc(-c4ccccc4)cc(-c4ccccc4)c3)c3cccc4c3oc3ccc5c(c34)-c3ccccc3C53c4ccccc4-c4ccccc43)cc2)cc1]

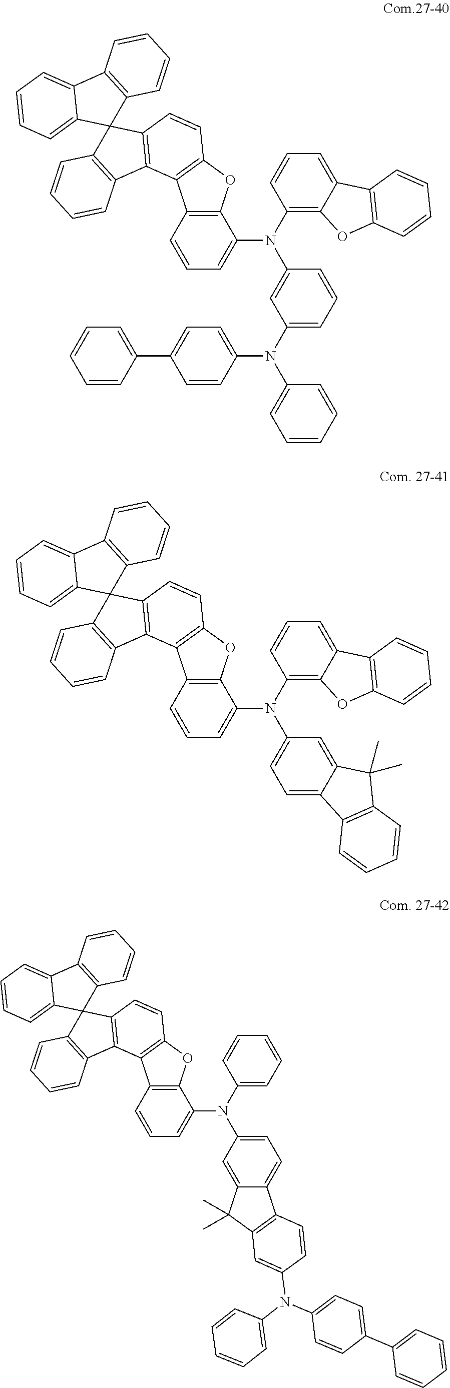 [SMILES: c1ccc(-c2ccc(N(c3ccccc3)c3cccc(N(c4cccc5c4oc4ccccc45)c4cccc5c4oc4ccc6c(c45)-c4ccccc4C64c5ccccc5-c5ccccc54)c3)cc2)cc1 ; CC1(C)c2ccccc2-c2ccc(N(c3cccc4c3oc3ccccc34)c3cccc4c3oc3ccc5c(c34)-c3ccccc3C53c4ccccc4-c4ccccc43)cc21 ; CC1(C)c2cc(N(c3ccccc3)c3ccc(-c4ccccc4)cc3)ccc2-c2ccc(N(c3ccccc3)c3cccc4c3oc3ccc5c(c34)-c3ccccc3C53c4ccccc4-c4ccccc43)cc21]

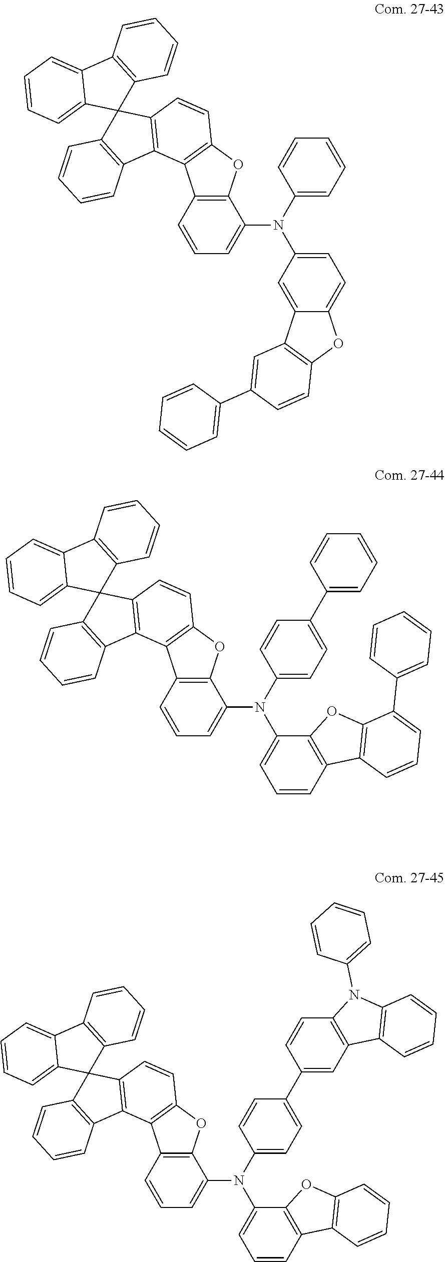 [SMILES: c1ccc(-c2ccc3oc4ccc(N(c5ccccc5)c5cccc6c5oc5ccc7c(c56)-c5ccccc5C75c6ccccc6-c6ccccc65)cc4c3c2)cc1 ; c1ccc(-c2ccc(N(c3cccc4c3oc3c(-c5ccccc5)cccc34)c3cccc4c3oc3ccc5c(c34)-c3ccccc3C53c4ccccc4-c4ccccc43)cc2)cc1 ; c1ccc(-n2c3ccccc3c3cc(-c4ccc(N(c5cccc6c5oc5ccccc56)c5cccc6c5oc5ccc7c(c56)-c5ccccc5C75c6ccccc6-c6ccccc65)cc4)ccc32)cc1]

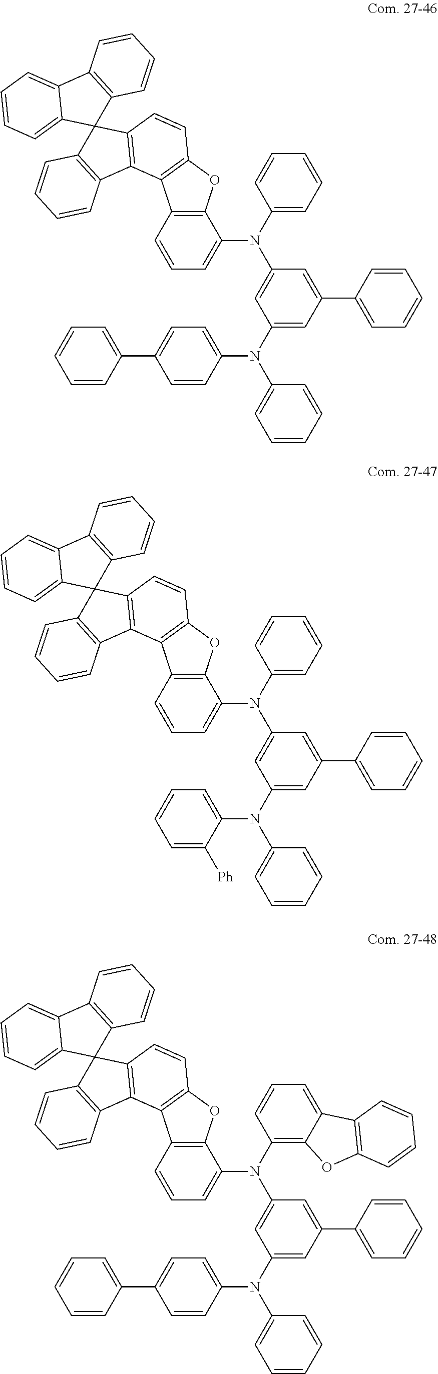 [SMILES: c1ccc(-c2ccc(N(c3ccccc3)c3cc(-c4ccccc4)cc(N(c4ccccc4)c4cccc5c4oc4ccc6c(c45)-c4ccccc4C64c5ccccc5-c5ccccc54)c3)cc2)cc1 ; c1ccc(-c2cc(N(c3ccccc3)c3ccccc3-c3ccccc3)cc(N(c3ccccc3)c3cccc4c3oc3ccc5c(c34)-c3ccccc3C53c4ccccc4-c4ccccc43)c2)cc1 ; c1ccc(-c2ccc(N(c3ccccc3)c3cc(-c4ccccc4)cc(N(c4cccc5c4oc4ccccc45)c4cccc5c4oc4ccc6c(c45)-c4ccccc4C64c5ccccc5-c5ccccc54)c3)cc2)cc1]

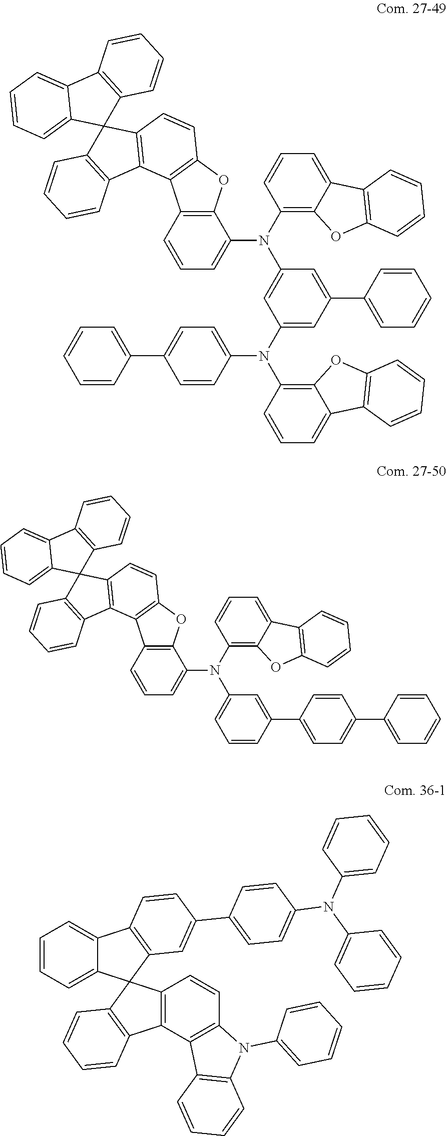 [SMILES: c1ccc(-c2ccc(N(c3cc(-c4ccccc4)cc(N(c4cccc5c4oc4ccccc45)c4cccc5c4oc4ccc6c(c45)-c4ccccc4C64c5ccccc5-c5ccccc54)c3)c3cccc4c3oc3ccccc34)cc2)cc1 ; c1ccc(-c2ccc(-c3cccc(N(c4cccc5c4oc4ccccc45)c4cccc5c4oc4ccc6c(c45)-c4ccccc4C64c5ccccc5-c5ccccc54)c3)cc2)cc1 ; c1ccc(N(c2ccccc2)c2ccc(-c3ccc4c(c3)C3(c5ccccc5-4)c4ccccc4-c4c3ccc3c4c4ccccc4n3-c3ccccc3)cc2)cc1]

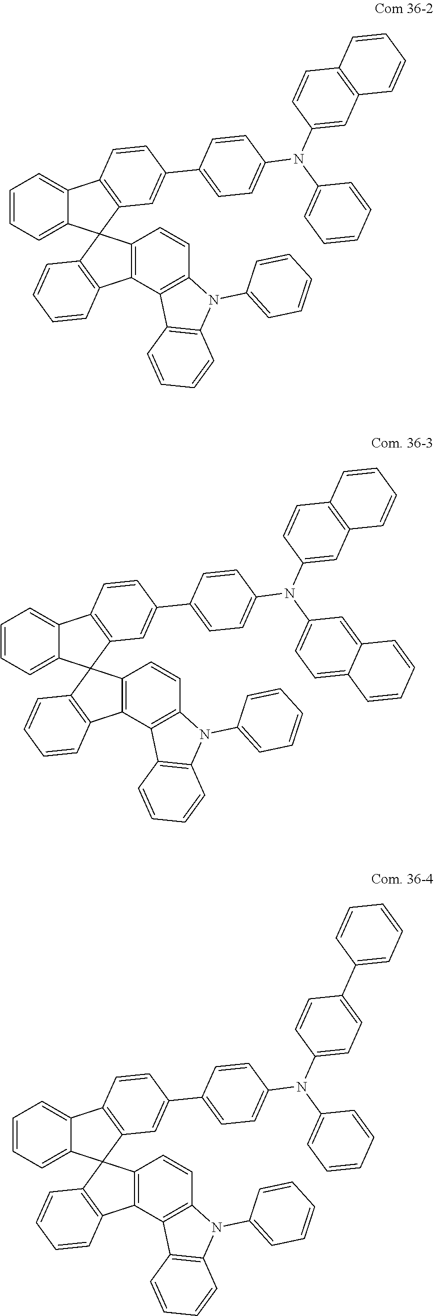 [SMILES: c1ccc(N(c2ccc(-c3ccc4c(c3)C3(c5ccccc5-4)c4ccccc4-c4c3ccc3c4c4ccccc4n3-c3ccccc3)cc2)c2ccc3ccccc3c2)cc1 ; c1ccc(-n2c3ccccc3c3c4c(ccc32)C2(c3ccccc3-c3ccc(-c5ccc(N(c6ccc7ccccc7c6)c6ccc7ccccc7c6)cc5)cc32)c2ccccc2-4)cc1 ; c1ccc(-c2ccc(N(c3ccccc3)c3ccc(-c4ccc5c(c4)C4(c6ccccc6-5)c5ccccc5-c5c4ccc4c5c5ccccc5n4-c4ccccc4)cc3)cc2)cc1]

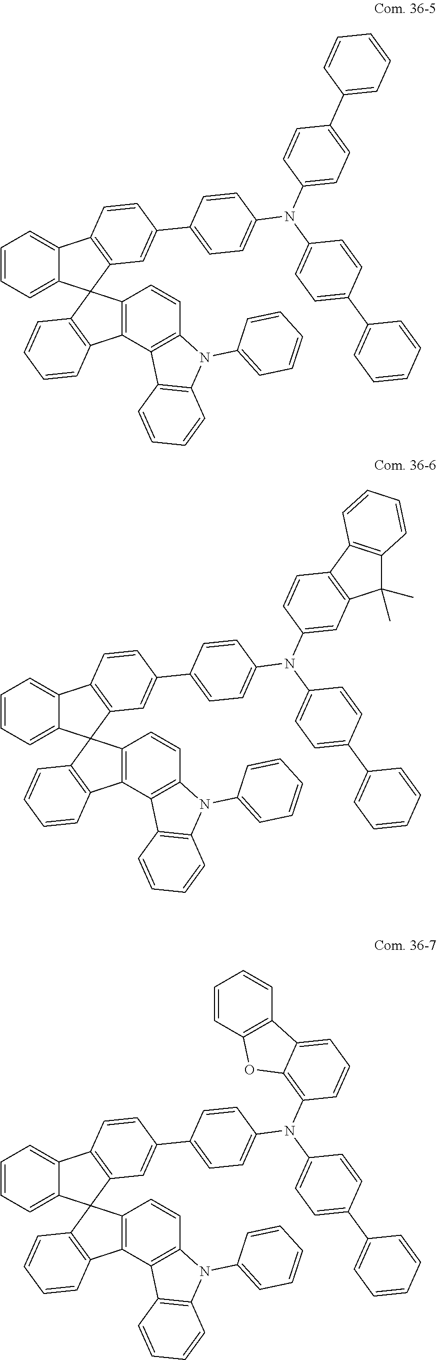 [SMILES: c1ccc(-c2ccc(N(c3ccc(-c4ccccc4)cc3)c3ccc(-c4ccc5c(c4)C4(c6ccccc6-5)c5ccccc5-c5c4ccc4c5c5ccccc5n4-c4ccccc4)cc3)cc2)cc1 ; CC1(C)c2ccccc2-c2ccc(N(c3ccc(-c4ccccc4)cc3)c3ccc(-c4ccc5c(c4)C4(c6ccccc6-5)c5ccccc5-c5c4ccc4c5c5ccccc5n4-c4ccccc4)cc3)cc21 ; c1ccc(-c2ccc(N(c3ccc(-c4ccc5c(c4)C4(c6ccccc6-5)c5ccccc5-c5c4ccc4c5c5ccccc5n4-c4ccccc4)cc3)c3cccc4c3oc3ccccc34)cc2)cc1]

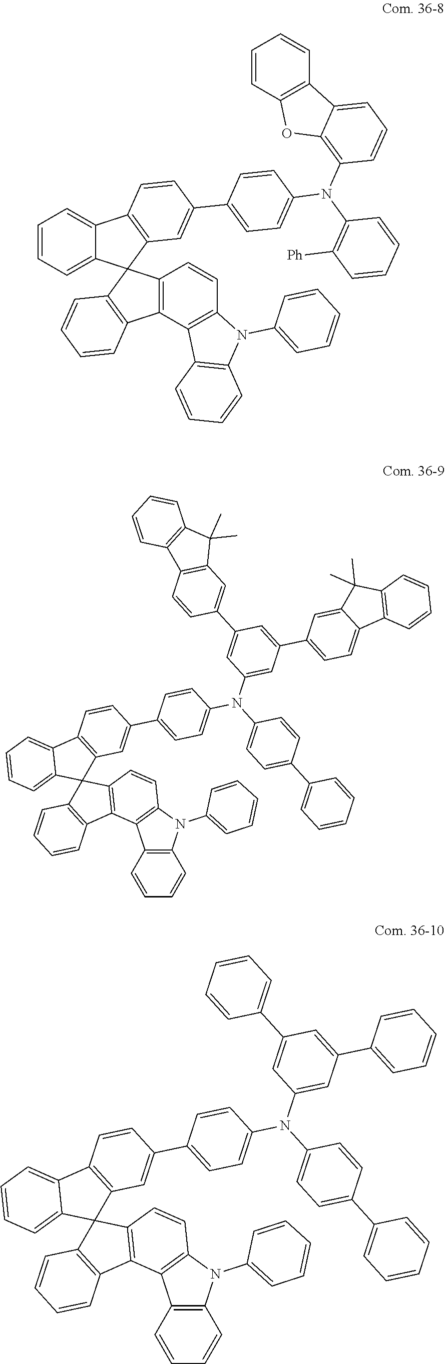 [SMILES: c1ccc(-c2ccccc2N(c2ccc(-c3ccc4c(c3)C3(c5ccccc5-4)c4ccccc4-c4c3ccc3c4c4ccccc4n3-c3ccccc3)cc2)c2cccc3c2oc2ccccc23)cc1 ; CC1(C)c2ccccc2-c2ccc(-c3cc(-c4ccc5c(c4)C(C)(C)c4ccccc4-5)cc(N(c4ccc(-c5ccccc5)cc4)c4ccc(-c5ccc6c(c5)C5(c7ccccc7-6)c6ccccc6-c6c5ccc5c6c6ccccc6n5-c5ccccc5)cc4)c3)cc21 ; c1ccc(-c2ccc(N(c3ccc(-c4ccc5c(c4)C4(c6ccccc6-5)c5ccccc5-c5c4ccc4c5c5ccccc5n4-c4ccccc4)cc3)c3cc(-c4ccccc4)cc(-c4ccccc4)c3)cc2)cc1]

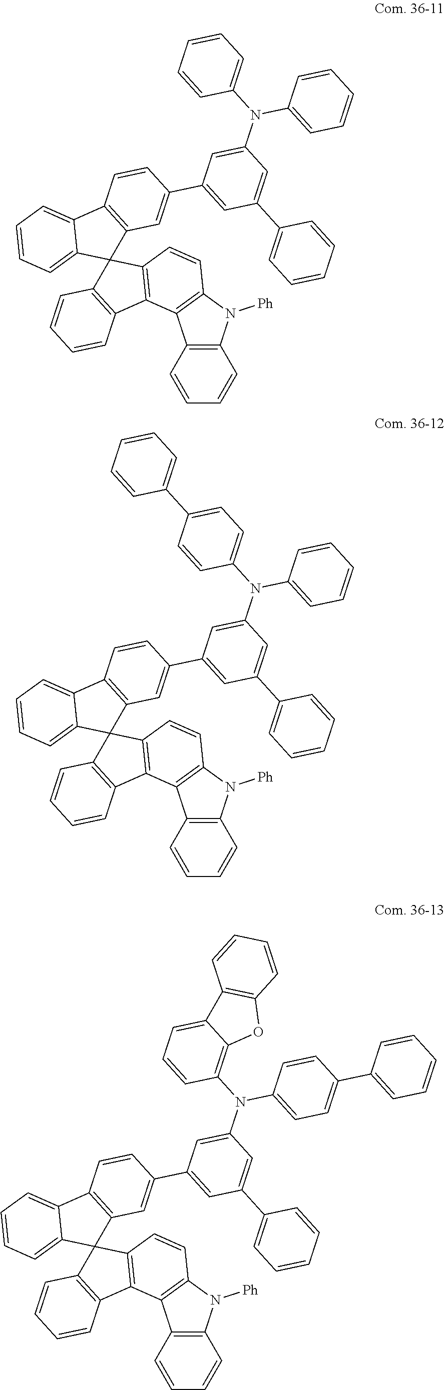

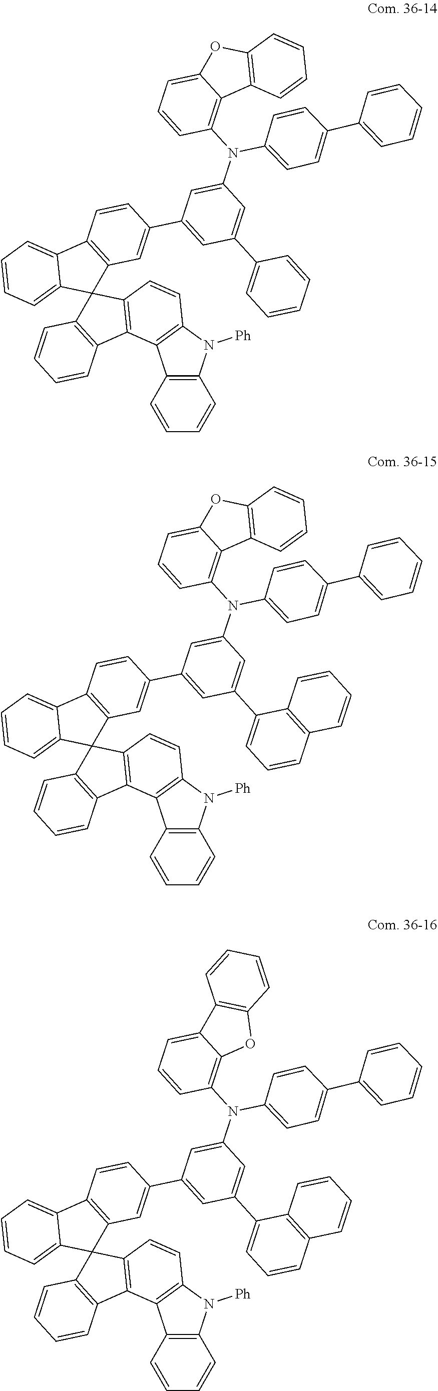 [SMILES: c1ccc(-c2ccc(N(c3cc(-c4ccccc4)cc(-c4ccc5c(c4)C4(c6ccccc6-5)c5ccccc5-c5c4ccc4c5c5ccccc5n4-c4ccccc4)c3)c3cccc4oc5ccccc5c34)cc2)cc1 ; c1ccc(-c2ccc(N(c3cc(-c4ccc5c(c4)C4(c6ccccc6-5)c5ccccc5-c5c4ccc4c5c5ccccc5n4-c4ccccc4)cc(-c4cccc5ccccc45)c3)c3cccc4oc5ccccc5c34)cc2)cc1 ; c1ccc(-c2ccc(N(c3cc(-c4ccc5c(c4)C4(c6ccccc6-5)c5ccccc5-c5c4ccc4c5c5ccccc5n4-c4ccccc4)cc(-c4cccc5ccccc45)c3)c3cccc4c3oc3ccccc34)cc2)cc1]

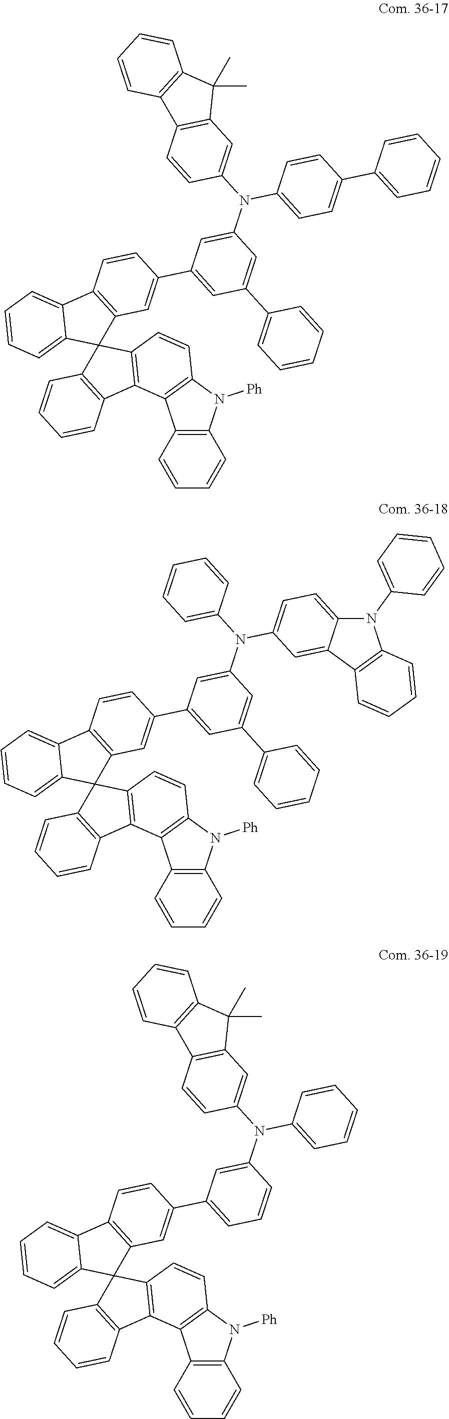

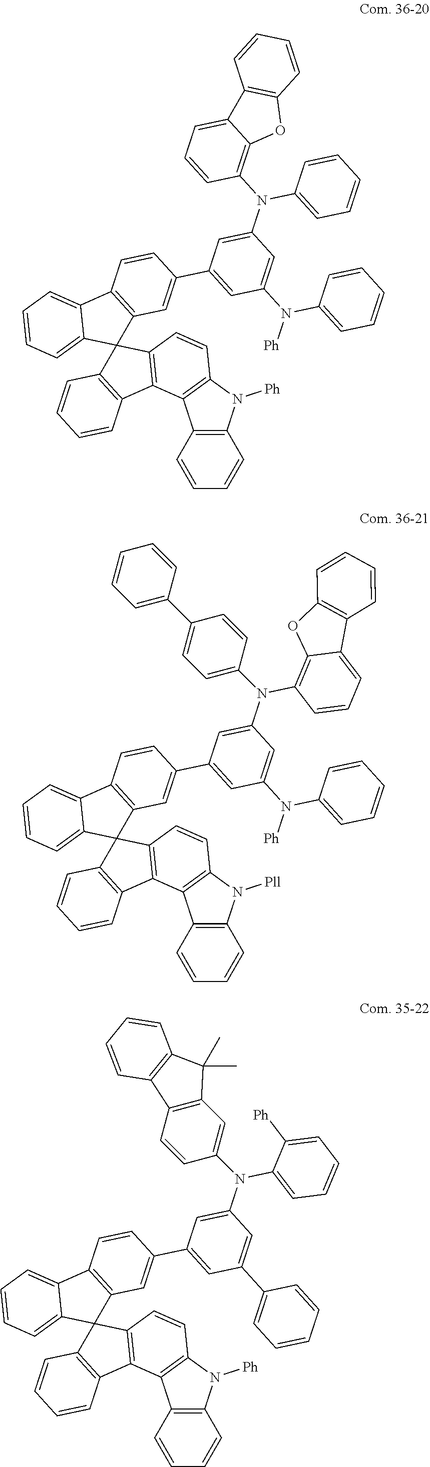

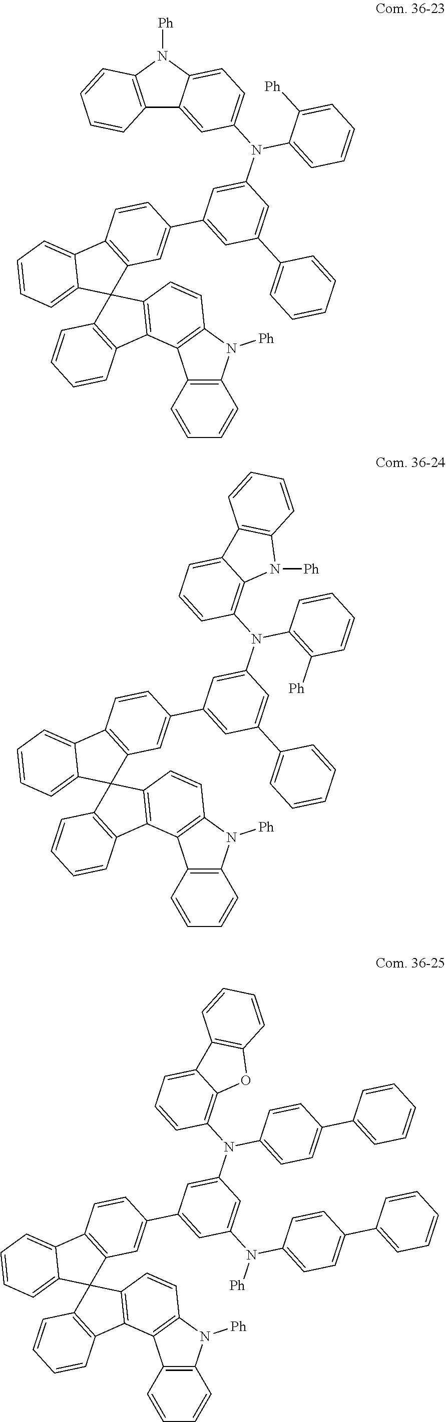

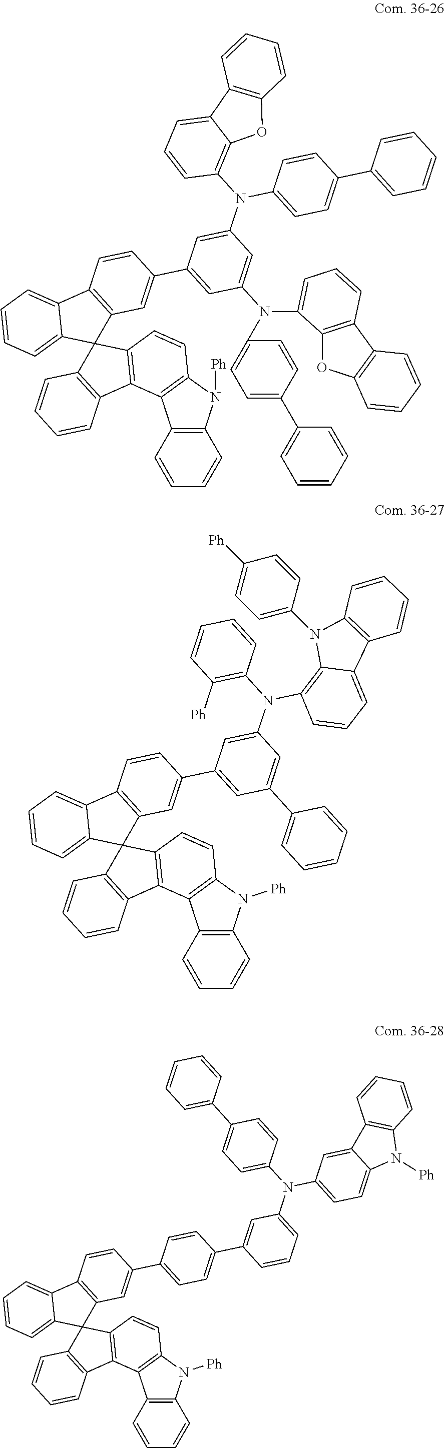 [SMILES: c1ccc(-c2ccc(N(c3cc(-c4ccc5c(c4)C4(c6ccccc6-5)c5ccccc5-c5c4ccc4c5c5ccccc5n4-c4ccccc4)cc(N(c4ccc(-c5ccccc5)cc4)c4cccc5c4oc4ccccc45)c3)c3cccc4c3oc3ccccc34)cc2)cc1 ; c1ccc(-c2ccc(-n3c4ccccc4c4cccc(N(c5cc(-c6ccccc6)cc(-c6ccc7c(c6)C6(c8ccccc8-7)c7ccccc7-c7c6ccc6c7c7ccccc7n6-c6ccccc6)c5)c5ccccc5-c5ccccc5)c43)cc2)cc1 ; c1ccc(-c2ccc(N(c3cccc(-c4ccc(-c5ccc6c(c5)C5(c7ccccc7-6)c6ccccc6-c6c5ccc5c6c6ccccc6n5-c5ccccc5)cc4)c3)c3ccc4c(c3)c3ccccc3n4-c3ccccc3)cc2)cc1]

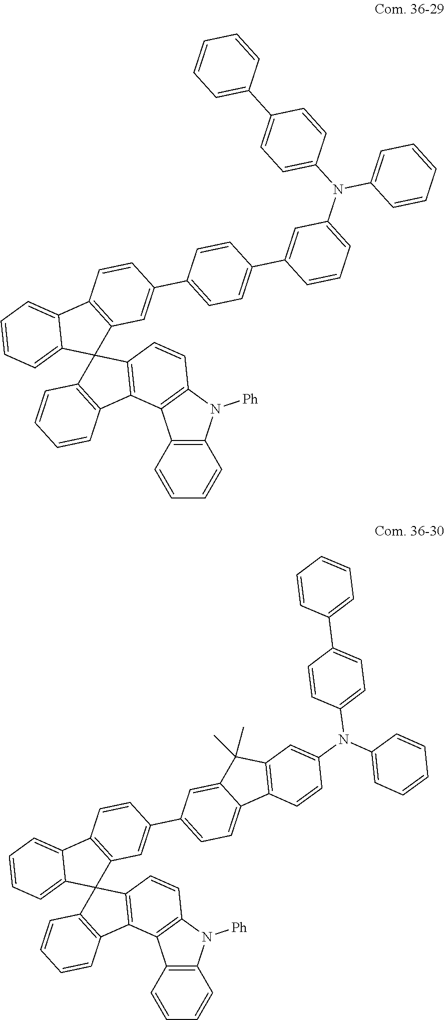 [SMILES: c1ccc(-c2ccc(N(c3ccccc3)c3cccc(-c4ccc(-c5ccc6c(c5)C5(c7ccccc7-6)c6ccccc6-c6c5ccc5c6c6ccccc6n5-c5ccccc5)cc4)c3)cc2)cc1 ; CC1(C)c2cc(-c3ccc4c(c3)C3(c5ccccc5-4)c4ccccc4-c4c3ccc3c4c4ccccc4n3-c3ccccc3)ccc2-c2ccc(N(c3ccccc3)c3ccc(-c4ccccc4)cc3)cc21]

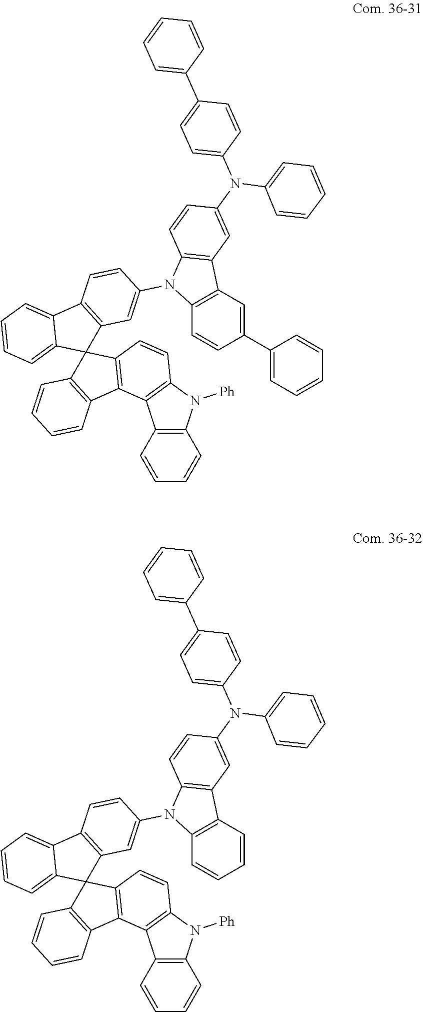 [SMILES: c1ccc(-c2ccc(N(c3ccccc3)c3ccc4c(c3)c3cc(-c5ccccc5)ccc3n4-c3ccc4c(c3)C3(c5ccccc5-4)c4ccccc4-c4c3ccc3c4c4ccccc4n3-c3ccccc3)cc2)cc1 ; c1ccc(-c2ccc(N(c3ccccc3)c3ccc4c(c3)c3ccccc3n4-c3ccc4c(c3)C3(c5ccccc5-4)c4ccccc4-c4c3ccc3c4c4ccccc4n3-c3ccccc3)cc2)cc1]

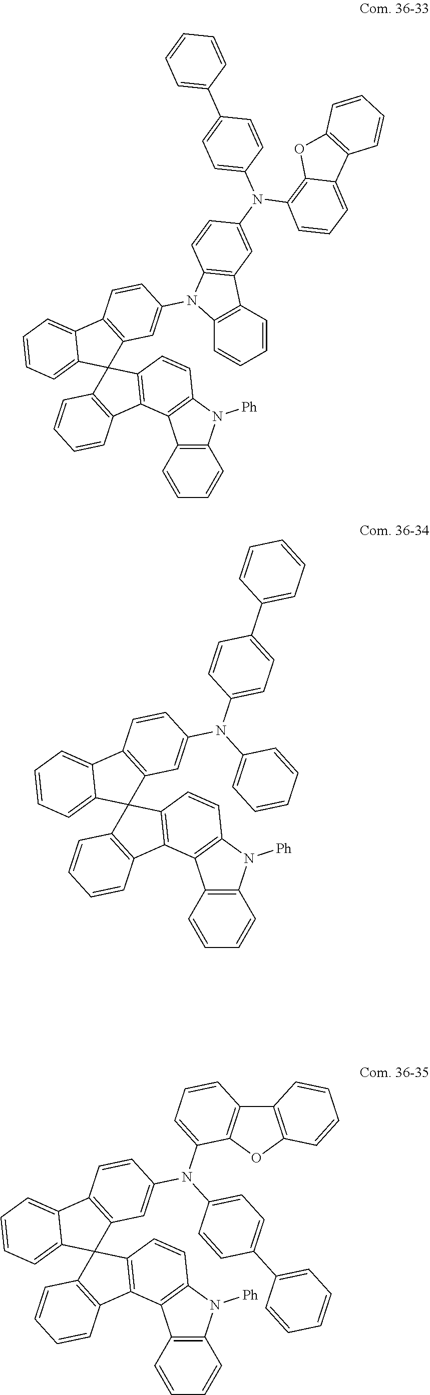 [SMILES: c1ccc(-c2ccc(N(c3ccc4c(c3)c3ccccc3n4-c3ccc4c(c3)C3(c5ccccc5-4)c4ccccc4-c4c3ccc3c4c4ccccc4n3-c3ccccc3)c3cccc4c3oc3ccccc34)cc2)cc1 ; c1ccc(-c2ccc(N(c3ccccc3)c3ccc4c(c3)C3(c5ccccc5-4)c4ccccc4-c4c3ccc3c4c4ccccc4n3-c3ccccc3)cc2)cc1 ; c1ccc(-c2ccc(N(c3ccc4c(c3)C3(c5ccccc5-4)c4ccccc4-c4c3ccc3c4c4ccccc4n3-c3ccccc3)c3cccc4c3oc3ccccc34)cc2)cc1]

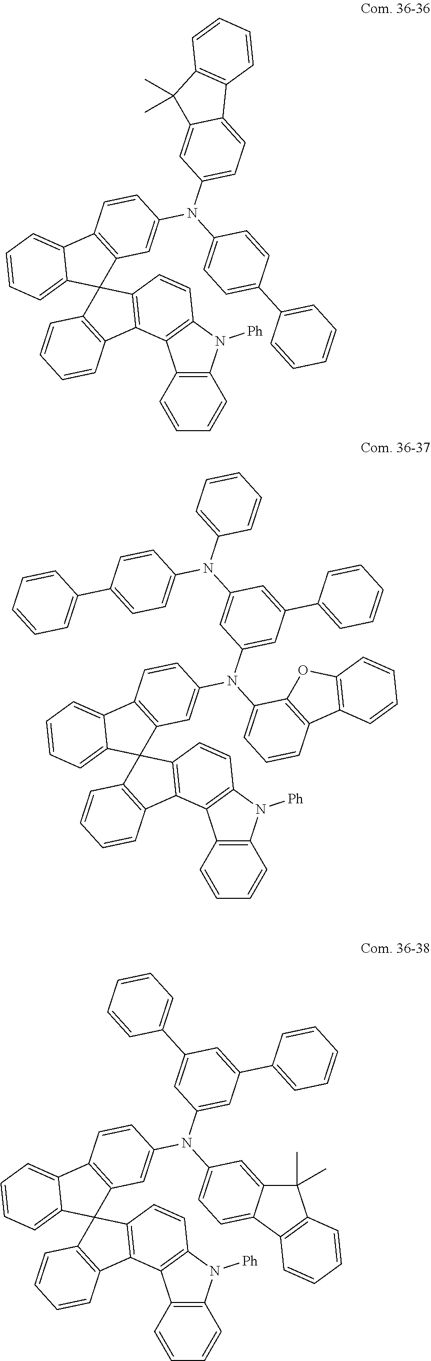 [SMILES: CC1(C)c2ccccc2-c2ccc(N(c3ccc(-c4ccccc4)cc3)c3ccc4c(c3)C3(c5ccccc5-4)c4ccccc4-c4c3ccc3c4c4ccccc4n3-c3ccccc3)cc21 ; c1ccc(-c2ccc(N(c3ccccc3)c3cc(-c4ccccc4)cc(N(c4ccc5c(c4)C4(c6ccccc6-5)c5ccccc5-c5c4ccc4c5c5ccccc5n4-c4ccccc4)c4cccc5c4oc4ccccc45)c3)cc2)cc1 ; CC1(C)c2ccccc2-c2ccc(N(c3cc(-c4ccccc4)cc(-c4ccccc4)c3)c3ccc4c(c3)C3(c5ccccc5-4)c4ccccc4-c4c3ccc3c4c4ccccc4n3-c3ccccc3)cc21]

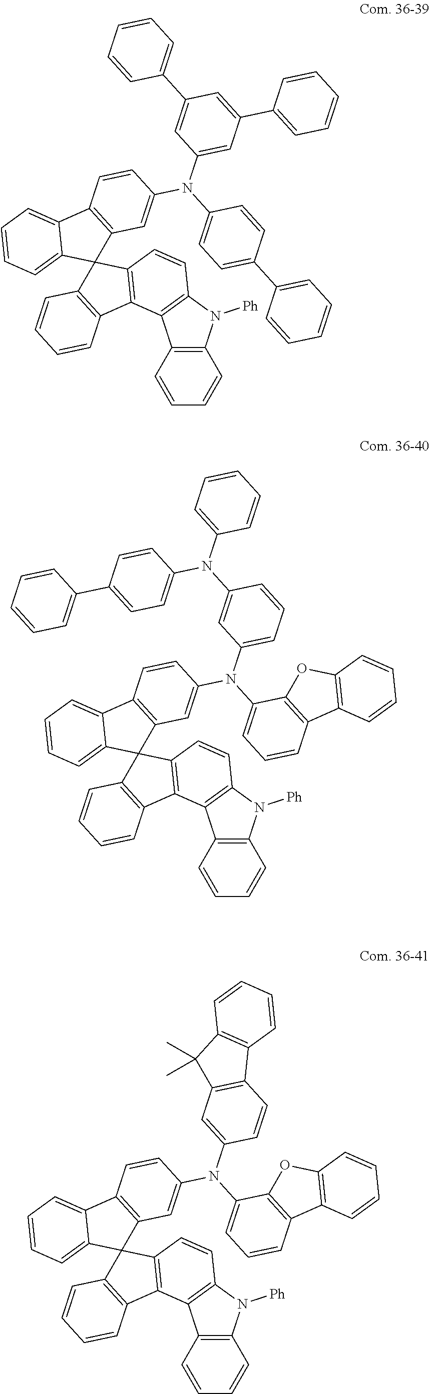 [SMILES: c1ccc(-c2ccc(N(c3cc(-c4ccccc4)cc(-c4ccccc4)c3)c3ccc4c(c3)C3(c5ccccc5-4)c4ccccc4-c4c3ccc3c4c4ccccc4n3-c3ccccc3)cc2)cc1 ; c1ccc(-c2ccc(N(c3ccccc3)c3cccc(N(c4ccc5c(c4)C4(c6ccccc6-5)c5ccccc5-c5c4ccc4c5c5ccccc5n4-c4ccccc4)c4cccc5c4oc4ccccc45)c3)cc2)cc1 ; CC1(C)c2ccccc2-c2ccc(N(c3ccc4c(c3)C3(c5ccccc5-4)c4ccccc4-c4c3ccc3c4c4ccccc4n3-c3ccccc3)c3cccc4c3oc3ccccc34)cc21]

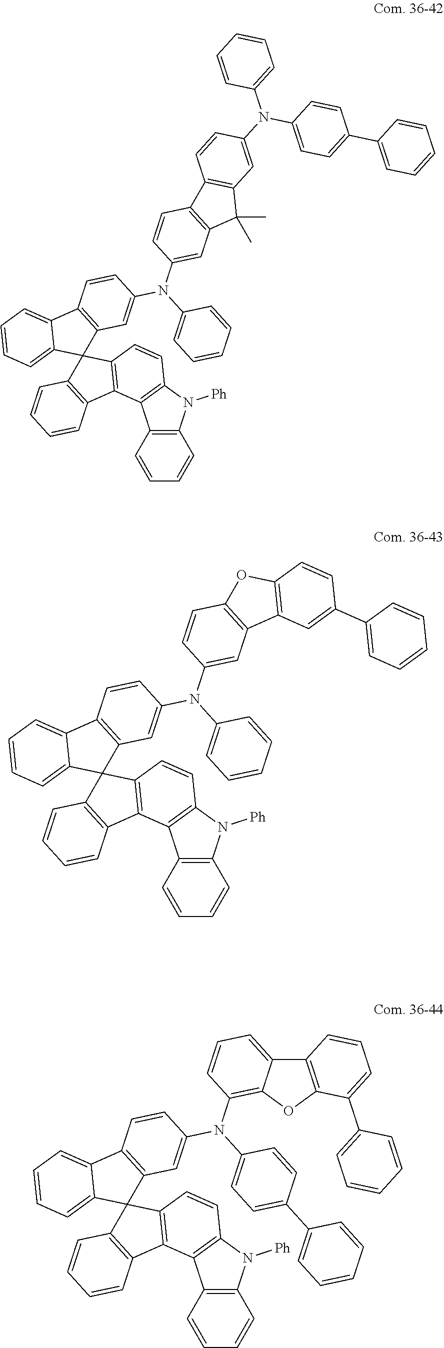 [SMILES: CC1(C)c2cc(N(c3ccccc3)c3ccc(-c4ccccc4)cc3)ccc2-c2ccc(N(c3ccccc3)c3ccc4c(c3)C3(c5ccccc5-4)c4ccccc4-c4c3ccc3c4c4ccccc4n3-c3ccccc3)cc21 ; c1ccc(-c2ccc3oc4ccc(N(c5ccccc5)c5ccc6c(c5)C5(c7ccccc7-6)c6ccccc6-c6c5ccc5c6c6ccccc6n5-c5ccccc5)cc4c3c2)cc1 ; c1ccc(-c2ccc(N(c3ccc4c(c3)C3(c5ccccc5-4)c4ccccc4-c4c3ccc3c4c4ccccc4n3-c3ccccc3)c3cccc4c3oc3c(-c5ccccc5)cccc34)cc2)cc1]

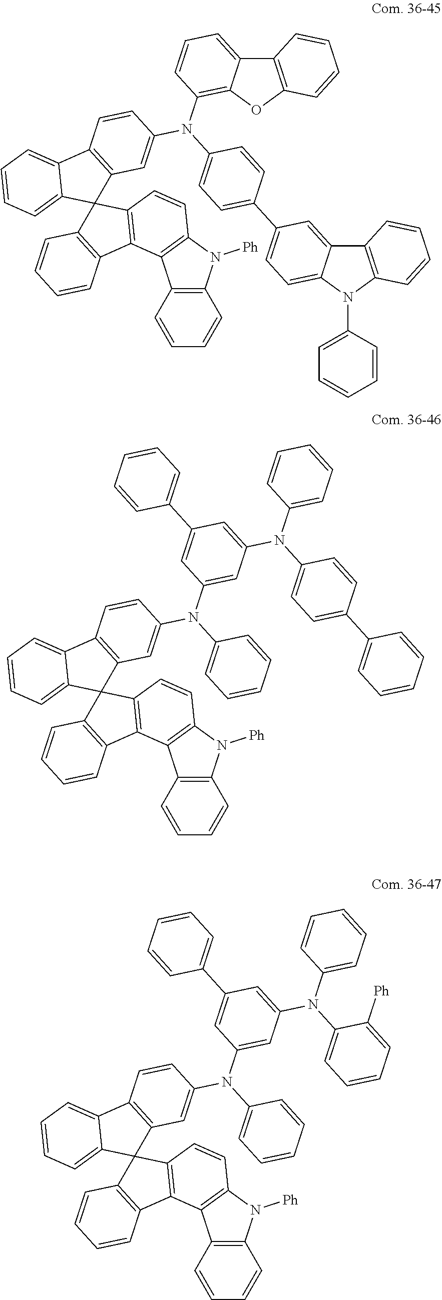 [SMILES: c1ccc(-n2c3ccccc3c3cc(-c4ccc(N(c5ccc6c(c5)C5(c7ccccc7-6)c6ccccc6-c6c5ccc5c6c6ccccc6n5-c5ccccc5)c5cccc6c5oc5ccccc56)cc4)ccc32)cc1 ; c1ccc(-c2ccc(N(c3ccccc3)c3cc(-c4ccccc4)cc(N(c4ccccc4)c4ccc5c(c4)C4(c6ccccc6-5)c5ccccc5-c5c4ccc4c5c5ccccc5n4-c4ccccc4)c3)cc2)cc1 ; c1ccc(-c2cc(N(c3ccccc3)c3ccc4c(c3)C3(c5ccccc5-4)c4ccccc4-c4c3ccc3c4c4ccccc4n3-c3ccccc3)cc(N(c3ccccc3)c3ccccc3-c3ccccc3)c2)cc1]

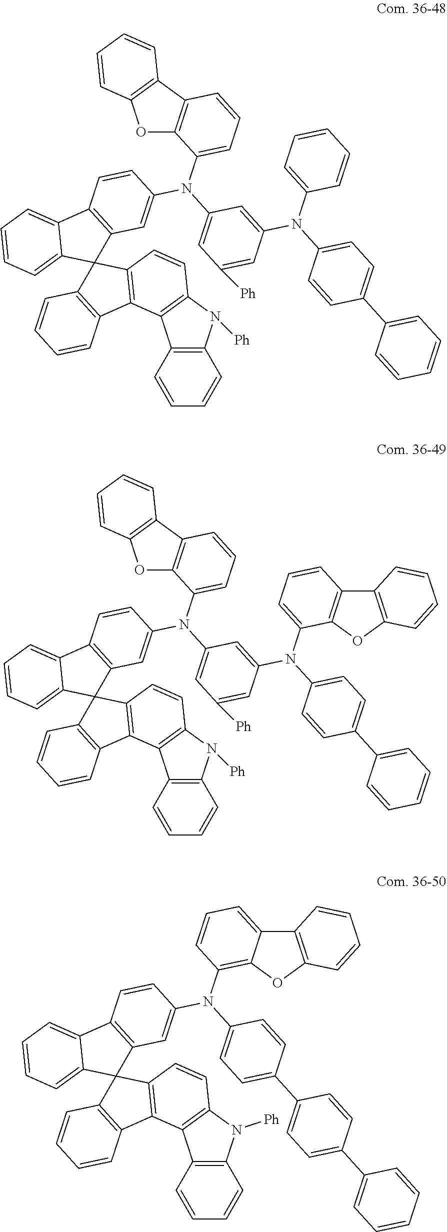 [SMILES: c1ccc(-c2ccc(N(c3ccccc3)c3cc(-c4ccccc4)cc(N(c4ccc5c(c4)C4(c6ccccc6-5)c5ccccc5-c5c4ccc4c5c5ccccc5n4-c4ccccc4)c4cccc5c4oc4ccccc45)c3)cc2)cc1 ; c1ccc(-c2ccc(N(c3cc(-c4ccccc4)cc(N(c4ccc5c(c4)C4(c6ccccc6-5)c5ccccc5-c5c4ccc4c5c5ccccc5n4-c4ccccc4)c4cccc5c4oc4ccccc45)c3)c3cccc4c3oc3ccccc34)cc2)cc1 ; c1ccc(-c2ccc(-c3ccc(N(c4ccc5c(c4)C4(c6ccccc6-5)c5ccccc5-c5c4ccc4c5c5ccccc5n4-c4ccccc4)c4cccc5c4oc4ccccc45)cc3)cc2)cc1]

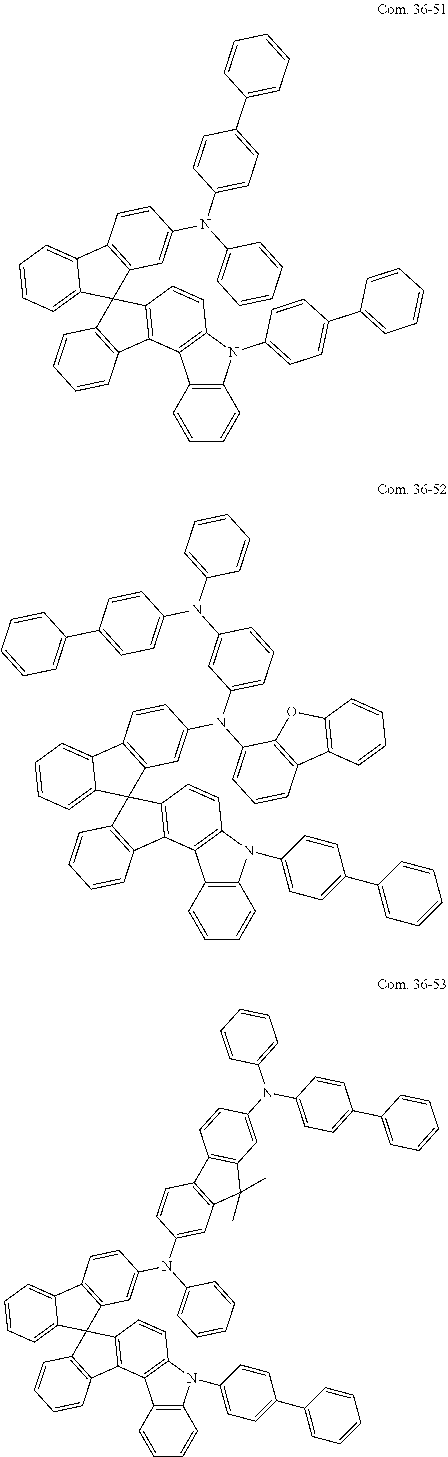 [SMILES: c1ccc(-c2ccc(N(c3ccccc3)c3ccc4c(c3)C3(c5ccccc5-4)c4ccccc4-c4c3ccc3c4c4ccccc4n3-c3ccc(-c4ccccc4)cc3)cc2)cc1 ; c1ccc(-c2ccc(N(c3ccccc3)c3cccc(N(c4ccc5c(c4)C4(c6ccccc6-5)c5ccccc5-c5c4ccc4c5c5ccccc5n4-c4ccc(-c5ccccc5)cc4)c4cccc5c4oc4ccccc45)c3)cc2)cc1 ; CC1(C)c2cc(N(c3ccccc3)c3ccc(-c4ccccc4)cc3)ccc2-c2ccc(N(c3ccccc3)c3ccc4c(c3)C3(c5ccccc5-4)c4ccccc4-c4c3ccc3c4c4ccccc4n3-c3ccc(-c4ccccc4)cc3)cc21]

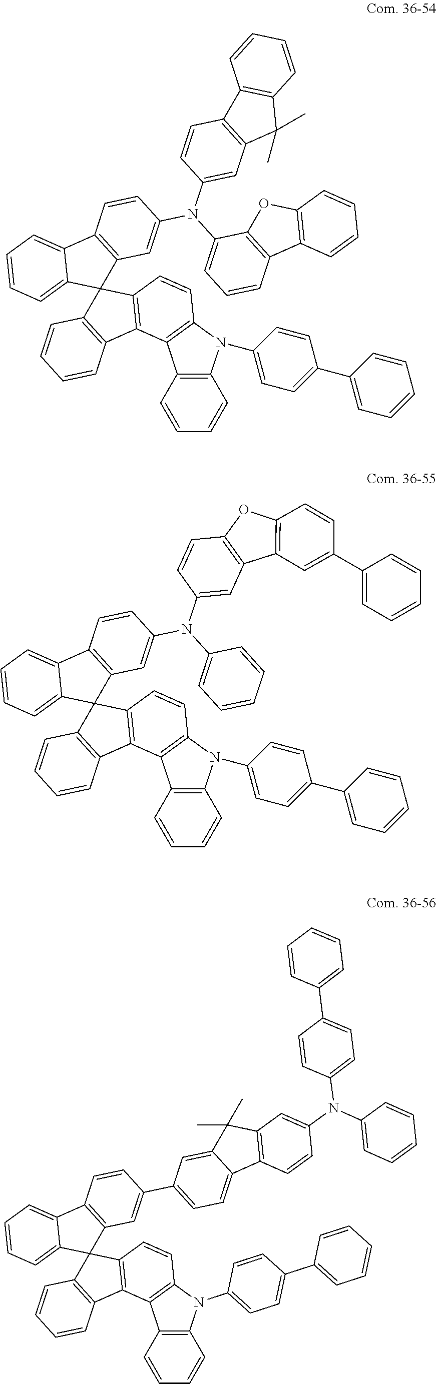 [SMILES: CC1(C)c2ccccc2-c2ccc(N(c3ccc4c(c3)C3(c5ccccc5-4)c4ccccc4-c4c3ccc3c4c4ccccc4n3-c3ccc(-c4ccccc4)cc3)c3cccc4c3oc3ccccc34)cc21 ; c1ccc(-c2ccc(-n3c4ccccc4c4c5c(ccc43)C3(c4ccccc4-c4ccc(N(c6ccccc6)c6ccc7oc8ccc(-c9ccccc9)cc8c7c6)cc43)c3ccccc3-5)cc2)cc1 ; CC1(C)c2cc(-c3ccc4c(c3)C3(c5ccccc5-4)c4ccccc4-c4c3ccc3c4c4ccccc4n3-c3ccc(-c4ccccc4)cc3)ccc2-c2ccc(N(c3ccccc3)c3ccc(-c4ccccc4)cc3)cc21]

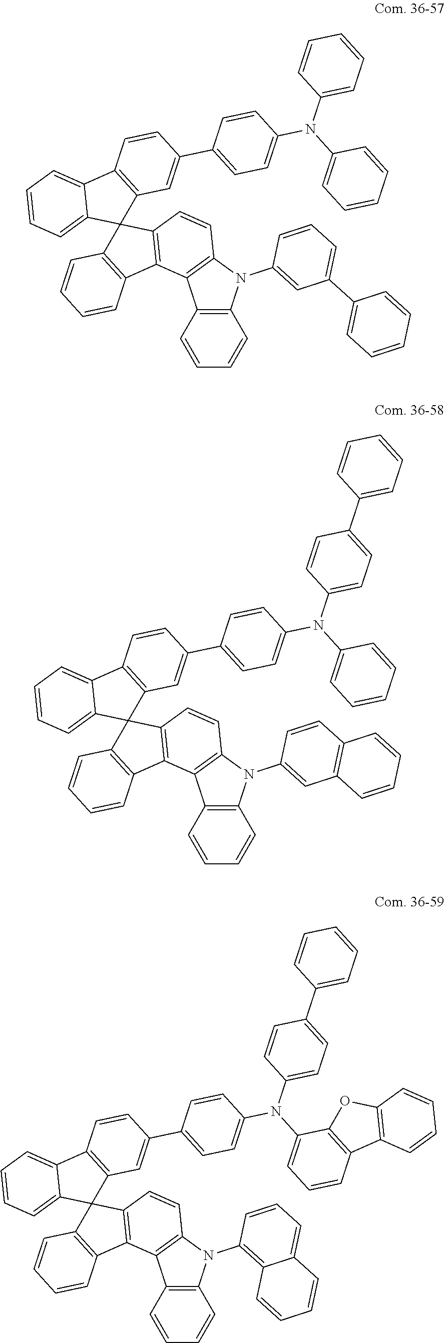 [SMILES: c1ccc(-c2cccc(-n3c4ccccc4c4c5c(ccc43)C3(c4ccccc4-c4ccc(-c6ccc(N(c7ccccc7)c7ccccc7)cc6)cc43)c3ccccc3-5)c2)cc1 ; c1ccc(-c2ccc(N(c3ccccc3)c3ccc(-c4ccc5c(c4)C4(c6ccccc6-5)c5ccccc5-c5c4ccc4c5c5ccccc5n4-c4ccc5ccccc5c4)cc3)cc2)cc1 ; c1ccc(-c2ccc(N(c3ccc(-c4ccc5c(c4)C4(c6ccccc6-5)c5ccccc5-c5c4ccc4c5c5ccccc5n4-c4cccc5ccccc45)cc3)c3cccc4c3oc3ccccc34)cc2)cc1]

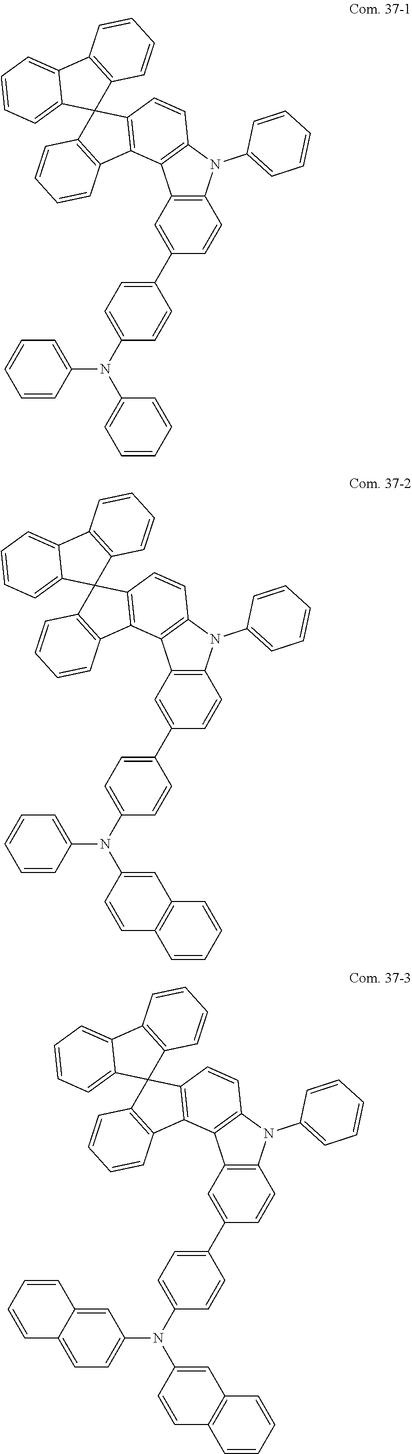

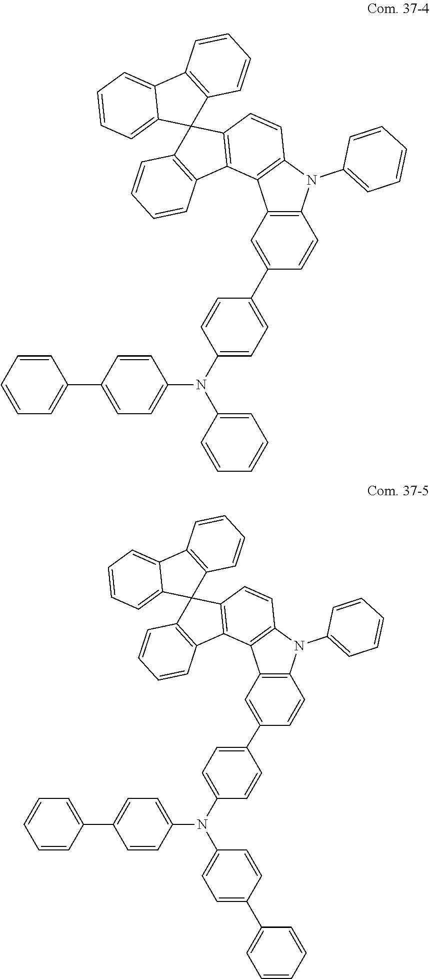 [SMILES: c1ccc(-c2ccc(N(c3ccccc3)c3ccc(-c4ccc5c(c4)c4c6c(ccc4n5-c4ccccc4)C4(c5ccccc5-c5ccccc54)c4ccccc4-6)cc3)cc2)cc1 ; c1ccc(-c2ccc(N(c3ccc(-c4ccccc4)cc3)c3ccc(-c4ccc5c(c4)c4c6c(ccc4n5-c4ccccc4)C4(c5ccccc5-c5ccccc54)c4ccccc4-6)cc3)cc2)cc1]

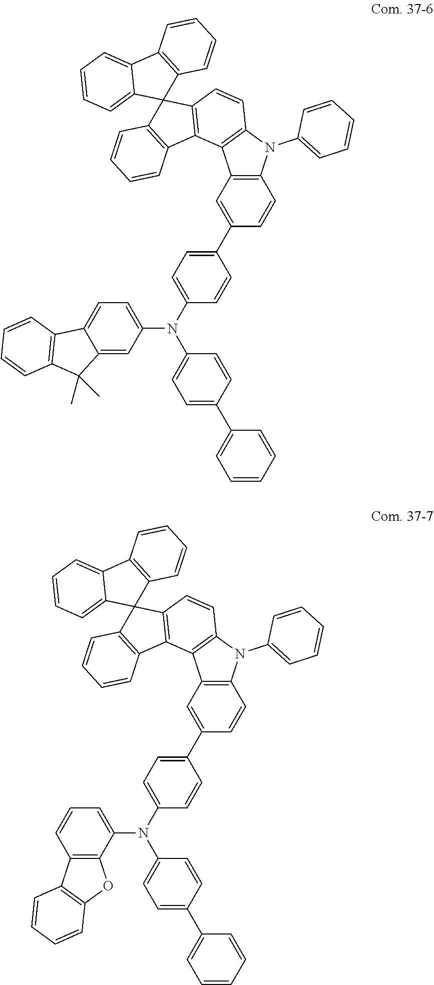 [SMILES: CC1(C)c2ccccc2-c2ccc(N(c3ccc(-c4ccccc4)cc3)c3ccc(-c4ccc5c(c4)c4c6c(ccc4n5-c4ccccc4)C4(c5ccccc5-c5ccccc54)c4ccccc4-6)cc3)cc21 ; c1ccc(-c2ccc(N(c3ccc(-c4ccc5c(c4)c4c6c(ccc4n5-c4ccccc4)C4(c5ccccc5-c5ccccc54)c4ccccc4-6)cc3)c3cccc4c3oc3ccccc34)cc2)cc1]

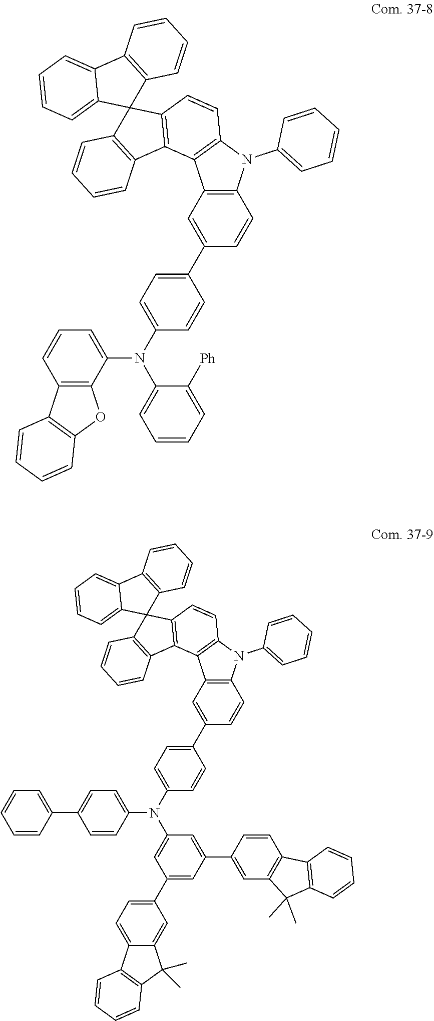 [SMILES: c1ccc(-c2ccccc2N(c2ccc(-c3ccc4c(c3)c3c5c(ccc3n4-c3ccccc3)C3(c4ccccc4-c4ccccc43)c3ccccc3-5)cc2)c2cccc3c2oc2ccccc23)cc1 ; CC1(C)c2ccccc2-c2ccc(-c3cc(-c4ccc5c(c4)C(C)(C)c4ccccc4-5)cc(N(c4ccc(-c5ccccc5)cc4)c4ccc(-c5ccc6c(c5)c5c7c(ccc5n6-c5ccccc5)C5(c6ccccc6-c6ccccc65)c5ccccc5-7)cc4)c3)cc21]

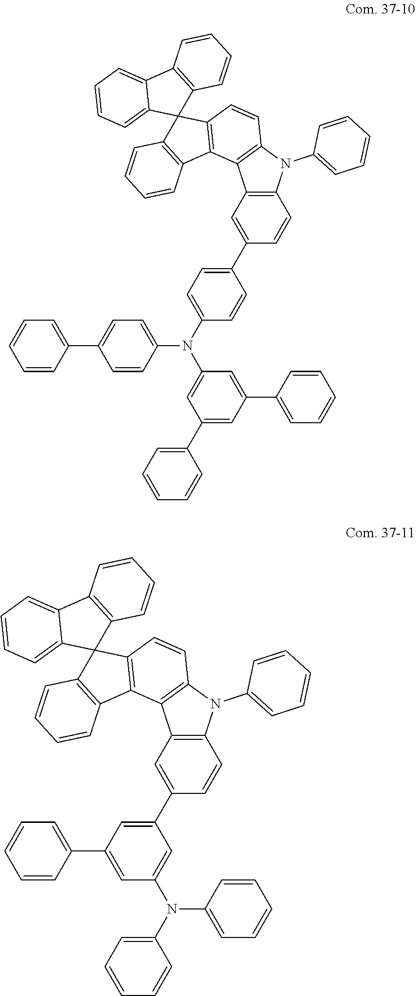 [SMILES: c1ccc(-c2ccc(N(c3ccc(-c4ccc5c(c4)c4c6c(ccc4n5-c4ccccc4)C4(c5ccccc5-c5ccccc54)c4ccccc4-6)cc3)c3cc(-c4ccccc4)cc(-c4ccccc4)c3)cc2)cc1 ; c1ccc(-c2cc(-c3ccc4c(c3)c3c5c(ccc3n4-c3ccccc3)C3(c4ccccc4-c4ccccc43)c3ccccc3-5)cc(N(c3ccccc3)c3ccccc3)c2)cc1]

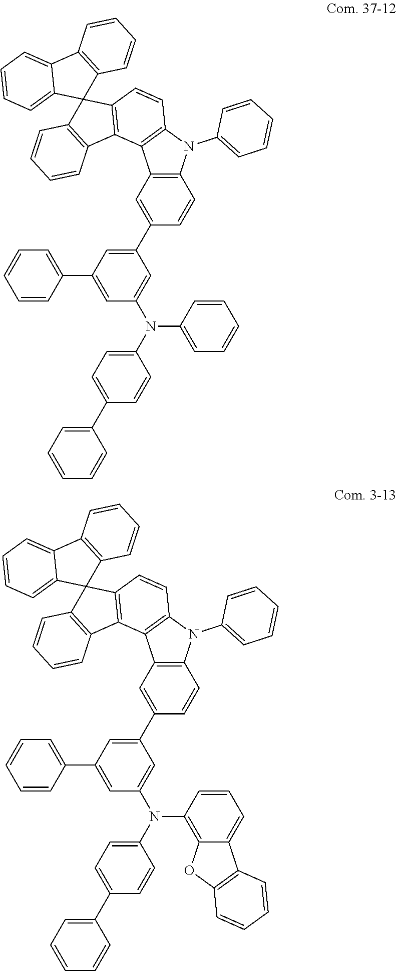

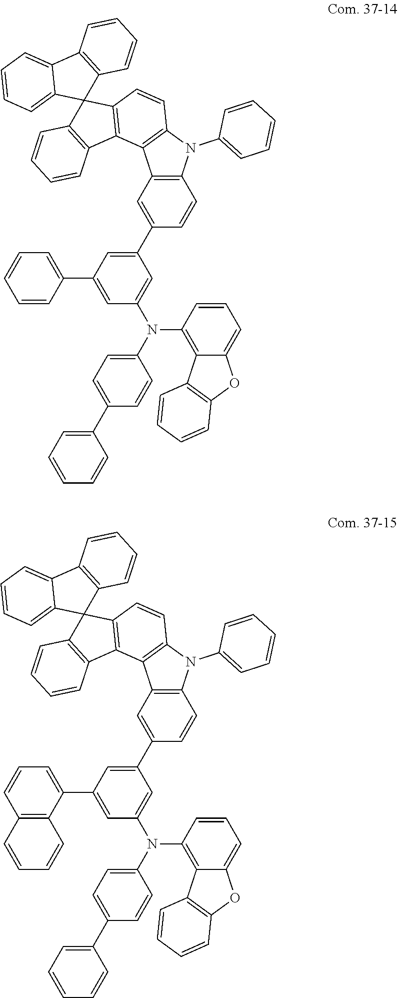 [SMILES: c1ccc(-c2ccc(N(c3cc(-c4ccccc4)cc(-c4ccc5c(c4)c4c6c(ccc4n5-c4ccccc4)C4(c5ccccc5-c5ccccc54)c4ccccc4-6)c3)c3cccc4oc5ccccc5c34)cc2)cc1 ; c1ccc(-c2ccc(N(c3cc(-c4ccc5c(c4)c4c6c(ccc4n5-c4ccccc4)C4(c5ccccc5-c5ccccc54)c4ccccc4-6)cc(-c4cccc5ccccc45)c3)c3cccc4oc5ccccc5c34)cc2)cc1]

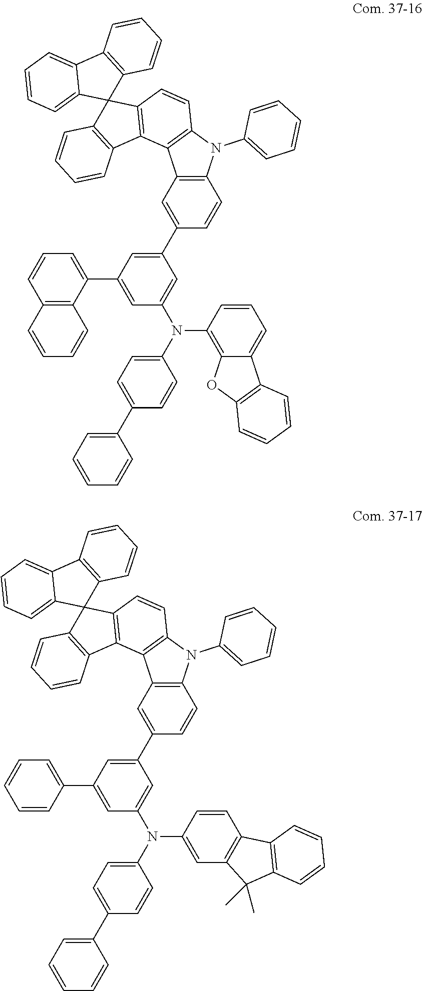

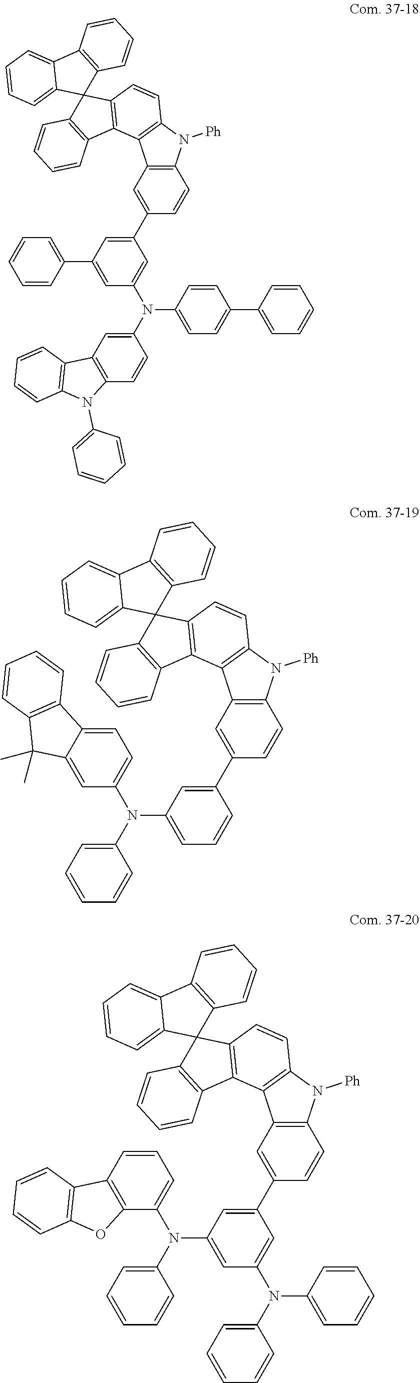 [SMILES: c1ccc(-c2ccc(N(c3cc(-c4ccccc4)cc(-c4ccc5c(c4)c4c6c(ccc4n5-c4ccccc4)C4(c5ccccc5-c5ccccc54)c4ccccc4-6)c3)c3ccc4c(c3)c3ccccc3n4-c3ccccc3)cc2)cc1 ; CC1(C)c2ccccc2-c2ccc(N(c3ccccc3)c3cccc(-c4ccc5c(c4)c4c6c(ccc4n5-c4ccccc4)C4(c5ccccc5-c5ccccc54)c4ccccc4-6)c3)cc21 ; c1ccc(N(c2ccccc2)c2cc(-c3ccc4c(c3)c3c5c(ccc3n4-c3ccccc3)C3(c4ccccc4-c4ccccc43)c3ccccc3-5)cc(N(c3ccccc3)c3cccc4c3oc3ccccc34)c2)cc1]

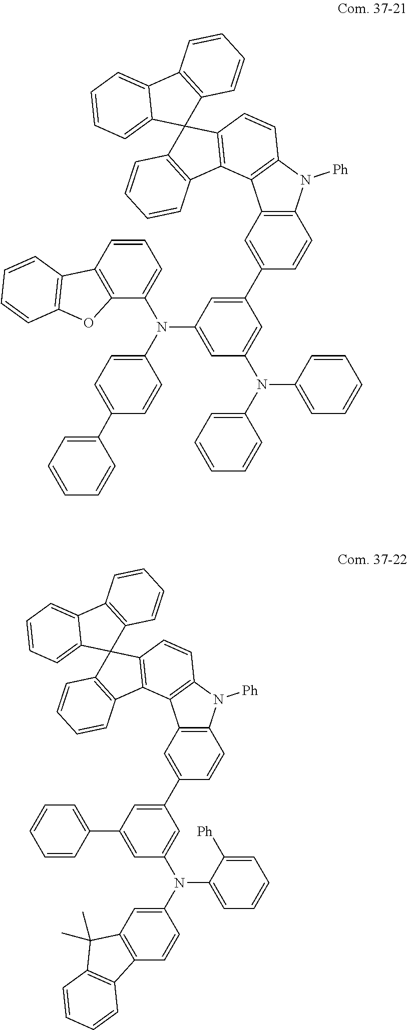 [SMILES: c1ccc(-c2ccc(N(c3cc(-c4ccc5c(c4)c4c6c(ccc4n5-c4ccccc4)C4(c5ccccc5-c5ccccc54)c4ccccc4-6)cc(N(c4ccccc4)c4ccccc4)c3)c3cccc4c3oc3ccccc34)cc2)cc1 ; CC1(C)c2ccccc2-c2ccc(N(c3cc(-c4ccccc4)cc(-c4ccc5c(c4)c4c6c(ccc4n5-c4ccccc4)C4(c5ccccc5-c5ccccc54)c4ccccc4-6)c3)c3ccccc3-c3ccccc3)cc21]

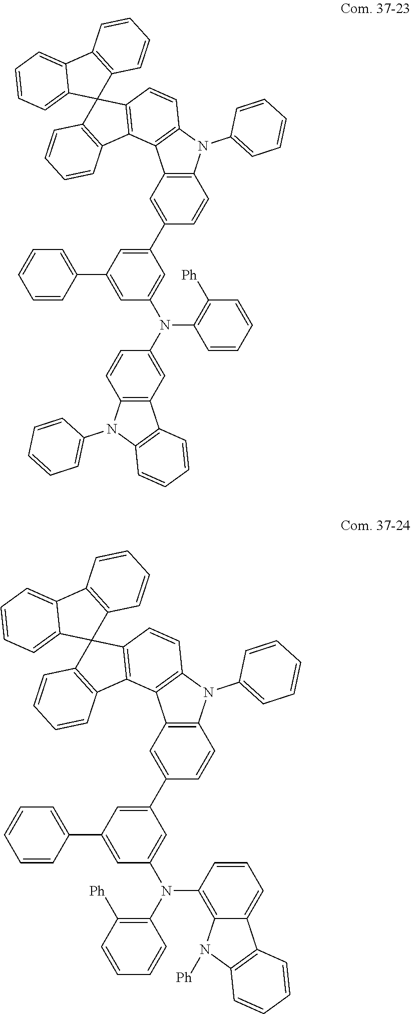 [SMILES: c1ccc(-c2cc(-c3ccc4c(c3)c3c5c(ccc3n4-c3ccccc3)C3(c4ccccc4-c4ccccc43)c3ccccc3-5)cc(N(c3ccc4c(c3)c3ccccc3n4-c3ccccc3)c3ccccc3-c3ccccc3)c2)cc1 ; c1ccc(-c2cc(-c3ccc4c(c3)c3c5c(ccc3n4-c3ccccc3)C3(c4ccccc4-c4ccccc43)c3ccccc3-5)cc(N(c3ccccc3-c3ccccc3)c3cccc4c5ccccc5n(-c5ccccc5)c34)c2)cc1]

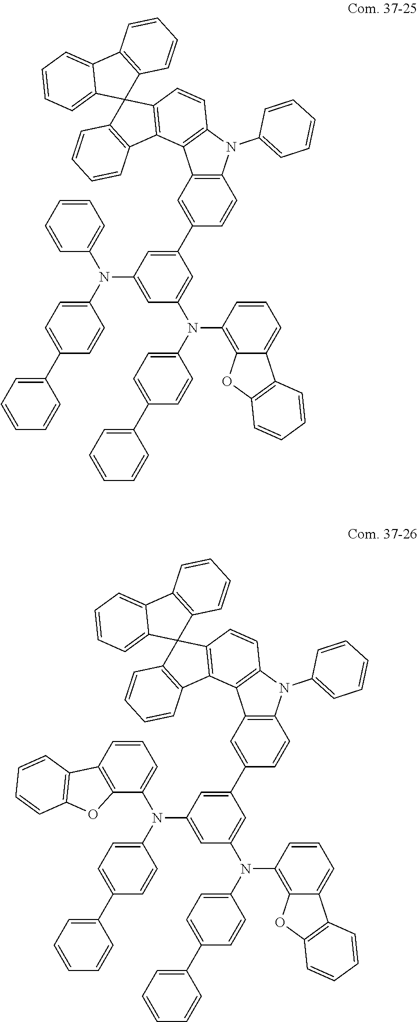 [SMILES: c1ccc(-c2ccc(N(c3ccccc3)c3cc(-c4ccc5c(c4)c4c6c(ccc4n5-c4ccccc4)C4(c5ccccc5-c5ccccc54)c4ccccc4-6)cc(N(c4ccc(-c5ccccc5)cc4)c4cccc5c4oc4ccccc45)c3)cc2)cc1 ; c1ccc(-c2ccc(N(c3cc(-c4ccc5c(c4)c4c6c(ccc4n5-c4ccccc4)C4(c5ccccc5-c5ccccc54)c4ccccc4-6)cc(N(c4ccc(-c5ccccc5)cc4)c4cccc5c4oc4ccccc45)c3)c3cccc4c3oc3ccccc34)cc2)cc1]

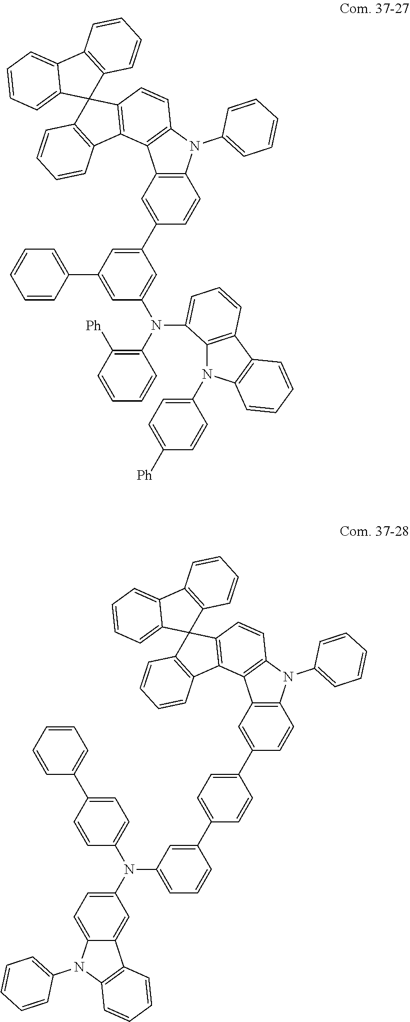 [SMILES: c1ccc(-c2ccc(-n3c4ccccc4c4cccc(N(c5cc(-c6ccccc6)cc(-c6ccc7c(c6)c6c8c(ccc6n7-c6ccccc6)C6(c7ccccc7-c7ccccc76)c6ccccc6-8)c5)c5ccccc5-c5ccccc5)c43)cc2)cc1 ; c1ccc(-c2ccc(N(c3cccc(-c4ccc(-c5ccc6c(c5)c5c7c(ccc5n6-c5ccccc5)C5(c6ccccc6-c6ccccc65)c5ccccc5-7)cc4)c3)c3ccc4c(c3)c3ccccc3n4-c3ccccc3)cc2)cc1]

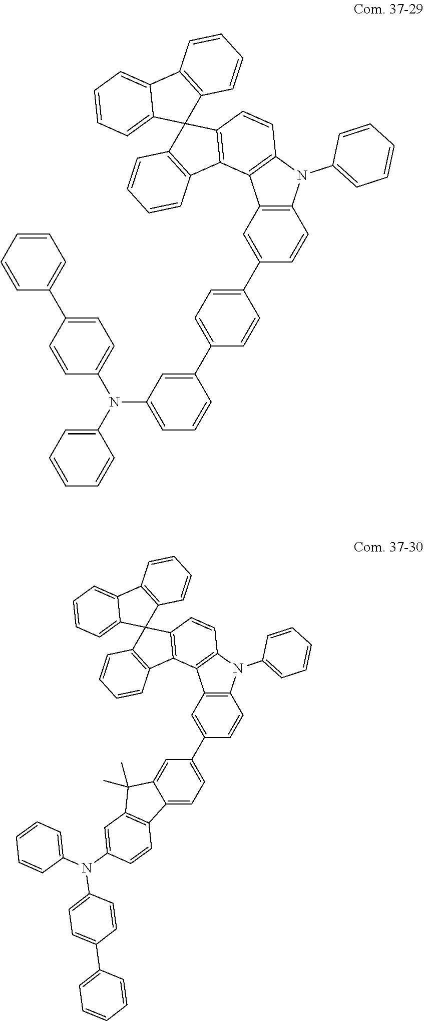 [SMILES: c1ccc(-c2ccc(N(c3ccccc3)c3cccc(-c4ccc(-c5ccc6c(c5)c5c7c(ccc5n6-c5ccccc5)C5(c6ccccc6-c6ccccc65)c5ccccc5-7)cc4)c3)cc2)cc1 ; CC1(C)c2cc(-c3ccc4c(c3)c3c5c(ccc3n4-c3ccccc3)C3(c4ccccc4-c4ccccc43)c3ccccc3-5)ccc2-c2ccc(N(c3ccccc3)c3ccc(-c4ccccc4)cc3)cc21]

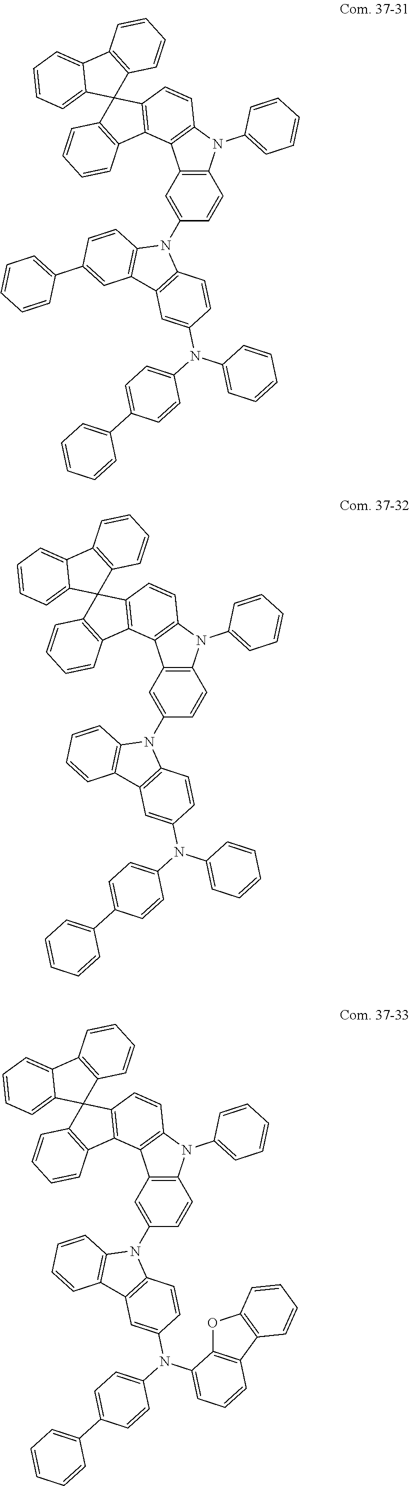 [SMILES: c1ccc(-c2ccc(N(c3ccccc3)c3ccc4c(c3)c3cc(-c5ccccc5)ccc3n4-c3ccc4c(c3)c3c5c(ccc3n4-c3ccccc3)C3(c4ccccc4-c4ccccc43)c3ccccc3-5)cc2)cc1 ; c1ccc(-c2ccc(N(c3ccccc3)c3ccc4c(c3)c3ccccc3n4-c3ccc4c(c3)c3c5c(ccc3n4-c3ccccc3)C3(c4ccccc4-c4ccccc43)c3ccccc3-5)cc2)cc1 ; c1ccc(-c2ccc(N(c3ccc4c(c3)c3ccccc3n4-c3ccc4c(c3)c3c5c(ccc3n4-c3ccccc3)C3(c4ccccc4-c4ccccc43)c3ccccc3-5)c3cccc4c3oc3ccccc34)cc2)cc1]

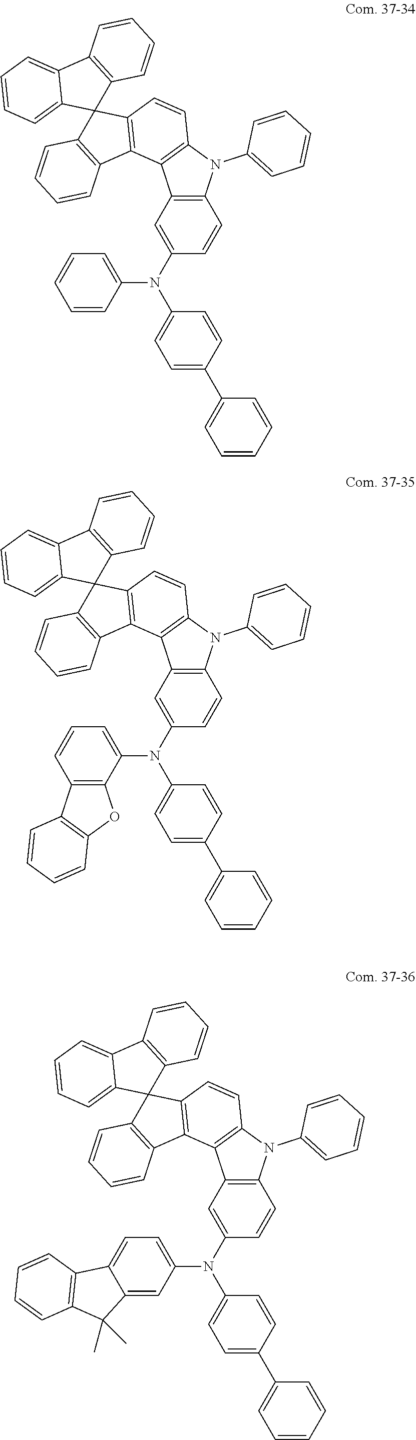 [SMILES: c1ccc(-c2ccc(N(c3ccccc3)c3ccc4c(c3)c3c5c(ccc3n4-c3ccccc3)C3(c4ccccc4-c4ccccc43)c3ccccc3-5)cc2)cc1 ; c1ccc(-c2ccc(N(c3ccc4c(c3)c3c5c(ccc3n4-c3ccccc3)C3(c4ccccc4-c4ccccc43)c3ccccc3-5)c3cccc4c3oc3ccccc34)cc2)cc1 ; CC1(C)c2ccccc2-c2ccc(N(c3ccc(-c4ccccc4)cc3)c3ccc4c(c3)c3c5c(ccc3n4-c3ccccc3)C3(c4ccccc4-c4ccccc43)c3ccccc3-5)cc21]

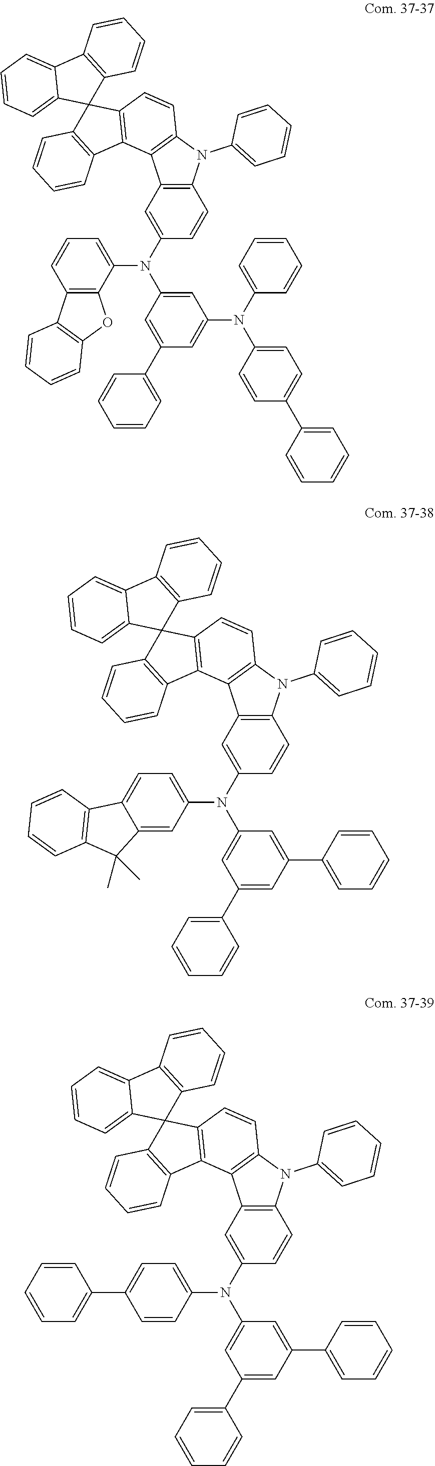 [SMILES: c1ccc(-c2ccc(N(c3ccccc3)c3cc(-c4ccccc4)cc(N(c4ccc5c(c4)c4c6c(ccc4n5-c4ccccc4)C4(c5ccccc5-c5ccccc54)c4ccccc4-6)c4cccc5c4oc4ccccc45)c3)cc2)cc1 ; CC1(C)c2ccccc2-c2ccc(N(c3cc(-c4ccccc4)cc(-c4ccccc4)c3)c3ccc4c(c3)c3c5c(ccc3n4-c3ccccc3)C3(c4ccccc4-c4ccccc43)c3ccccc3-5)cc21 ; c1ccc(-c2ccc(N(c3cc(-c4ccccc4)cc(-c4ccccc4)c3)c3ccc4c(c3)c3c5c(ccc3n4-c3ccccc3)C3(c4ccccc4-c4ccccc43)c3ccccc3-5)cc2)cc1]

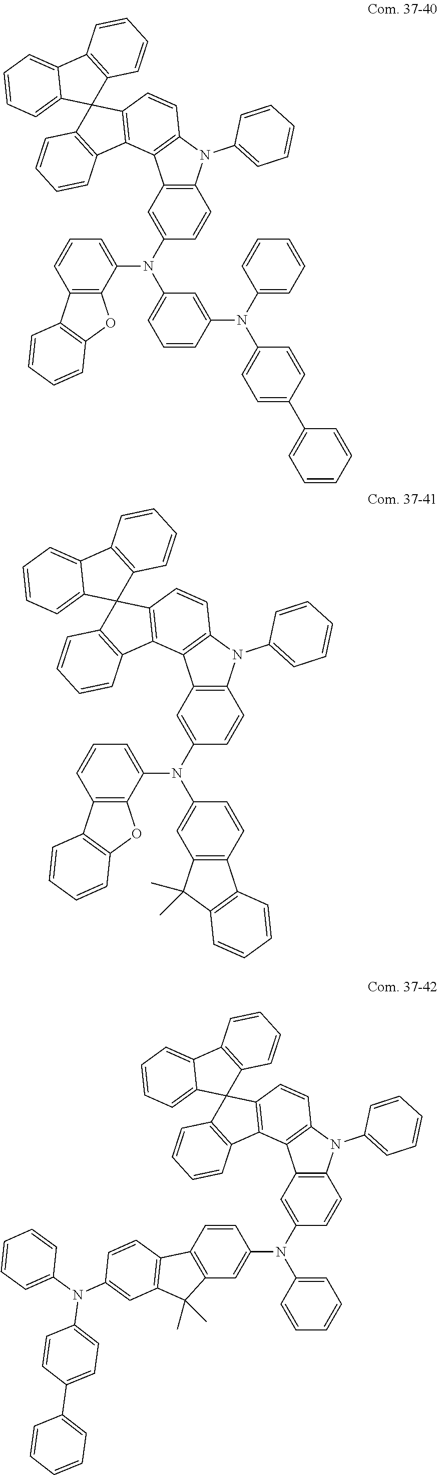 [SMILES: c1ccc(-c2ccc(N(c3ccccc3)c3cccc(N(c4ccc5c(c4)c4c6c(ccc4n5-c4ccccc4)C4(c5ccccc5-c5ccccc54)c4ccccc4-6)c4cccc5c4oc4ccccc45)c3)cc2)cc1 ; CC1(C)c2ccccc2-c2ccc(N(c3ccc4c(c3)c3c5c(ccc3n4-c3ccccc3)C3(c4ccccc4-c4ccccc43)c3ccccc3-5)c3cccc4c3oc3ccccc34)cc21 ; CC1(C)c2cc(N(c3ccccc3)c3ccc(-c4ccccc4)cc3)ccc2-c2ccc(N(c3ccccc3)c3ccc4c(c3)c3c5c(ccc3n4-c3ccccc3)C3(c4ccccc4-c4ccccc43)c3ccccc3-5)cc21]

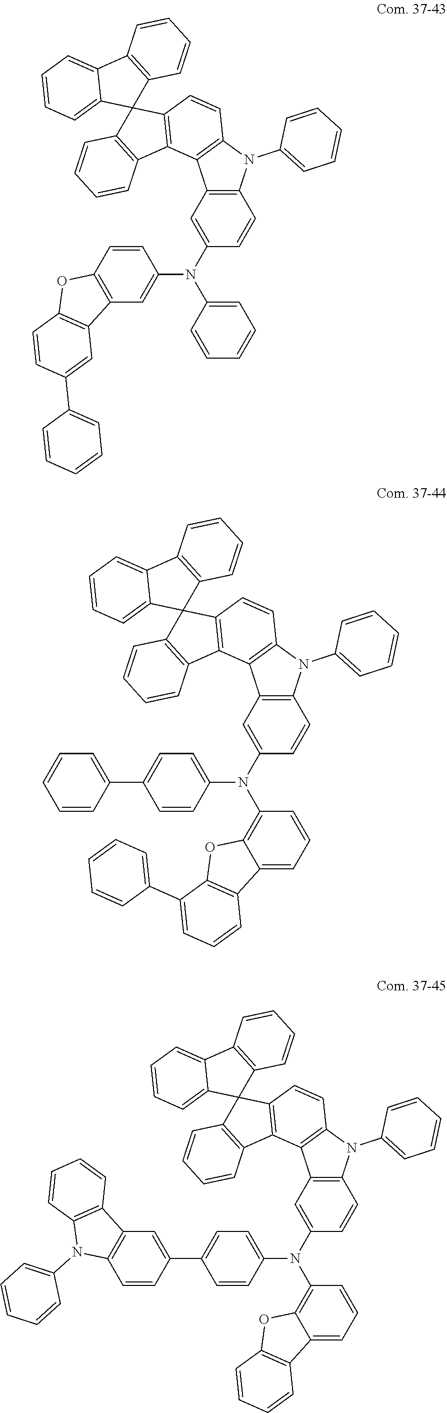 [SMILES: c1ccc(-c2ccc3oc4ccc(N(c5ccccc5)c5ccc6c(c5)c5c7c(ccc5n6-c5ccccc5)C5(c6ccccc6-c6ccccc65)c5ccccc5-7)cc4c3c2)cc1 ; c1ccc(-c2ccc(N(c3ccc4c(c3)c3c5c(ccc3n4-c3ccccc3)C3(c4ccccc4-c4ccccc43)c3ccccc3-5)c3cccc4c3oc3c(-c5ccccc5)cccc34)cc2)cc1 ; c1ccc(-n2c3ccccc3c3cc(-c4ccc(N(c5ccc6c(c5)c5c7c(ccc5n6-c5ccccc5)C5(c6ccccc6-c6ccccc65)c5ccccc5-7)c5cccc6c5oc5ccccc56)cc4)ccc32)cc1]

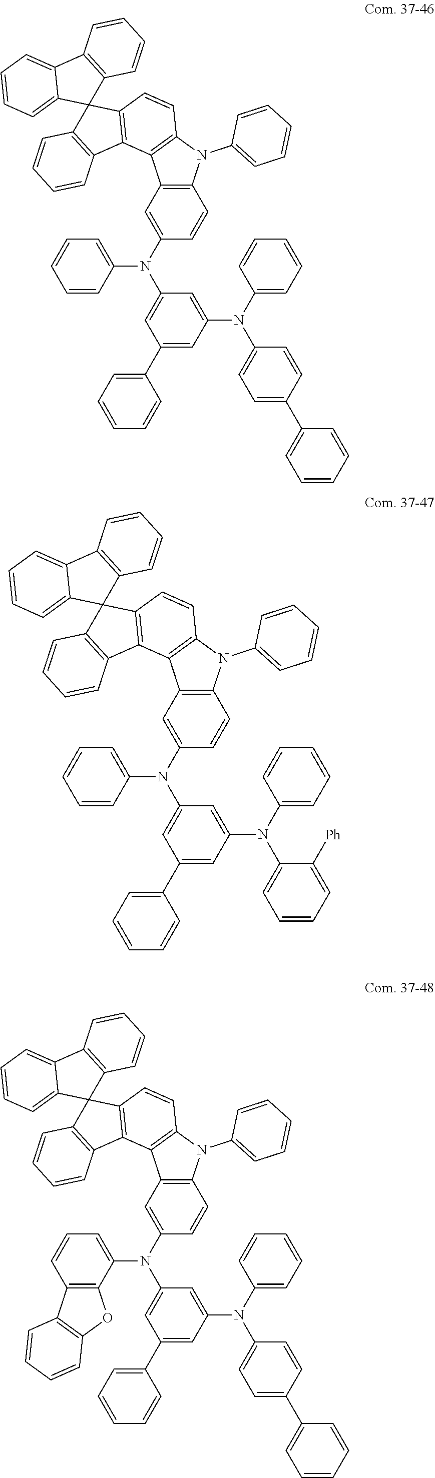 [SMILES: c1ccc(-c2ccc(N(c3ccccc3)c3cc(-c4ccccc4)cc(N(c4ccccc4)c4ccc5c(c4)c4c6c(ccc4n5-c4ccccc4)C4(c5ccccc5-c5ccccc54)c4ccccc4-6)c3)cc2)cc1 ; c1ccc(-c2cc(N(c3ccccc3)c3ccc4c(c3)c3c5c(ccc3n4-c3ccccc3)C3(c4ccccc4-c4ccccc43)c3ccccc3-5)cc(N(c3ccccc3)c3ccccc3-c3ccccc3)c2)cc1 ; c1ccc(-c2ccc(N(c3ccccc3)c3cc(-c4ccccc4)cc(N(c4ccc5c(c4)c4c6c(ccc4n5-c4ccccc4)C4(c5ccccc5-c5ccccc54)c4ccccc4-6)c4cccc5c4oc4ccccc45)c3)cc2)cc1]

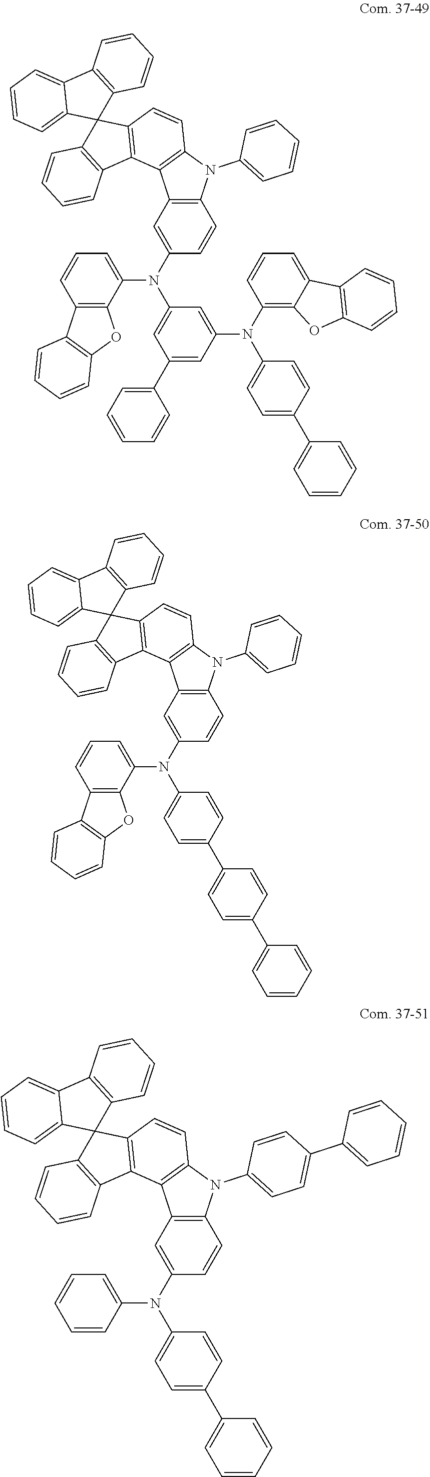 [SMILES: c1ccc(-c2ccc(N(c3cc(-c4ccccc4)cc(N(c4ccc5c(c4)c4c6c(ccc4n5-c4ccccc4)C4(c5ccccc5-c5ccccc54)c4ccccc4-6)c4cccc5c4oc4ccccc45)c3)c3cccc4c3oc3ccccc34)cc2)cc1 ; c1ccc(-c2ccc(-c3ccc(N(c4ccc5c(c4)c4c6c(ccc4n5-c4ccccc4)C4(c5ccccc5-c5ccccc54)c4ccccc4-6)c4cccc5c4oc4ccccc45)cc3)cc2)cc1 ; c1ccc(-c2ccc(N(c3ccccc3)c3ccc4c(c3)c3c5c(ccc3n4-c3ccc(-c4ccccc4)cc3)C3(c4ccccc4-c4ccccc43)c3ccccc3-5)cc2)cc1]

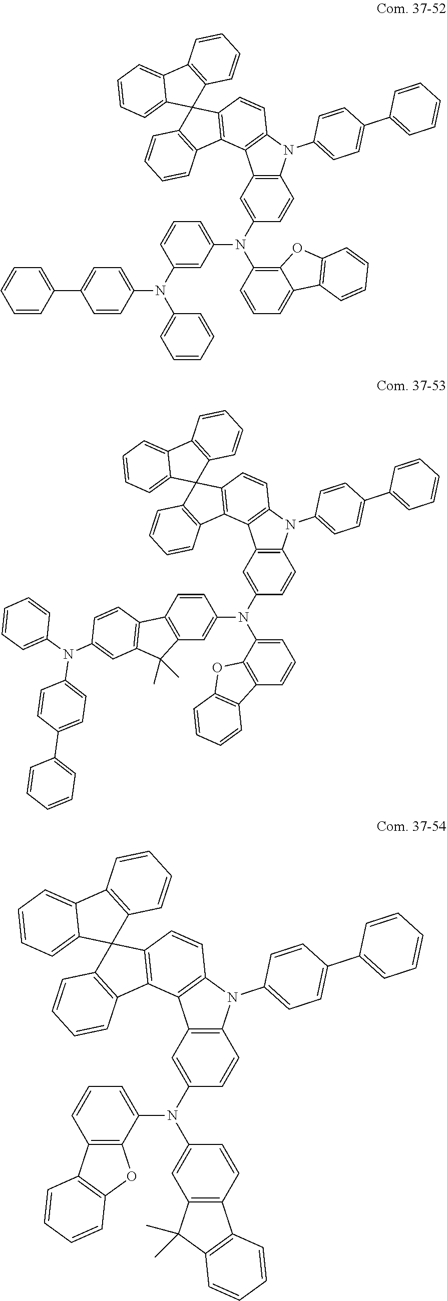 [SMILES: c1ccc(-c2ccc(N(c3ccccc3)c3cccc(N(c4ccc5c(c4)c4c6c(ccc4n5-c4ccc(-c5ccccc5)cc4)C4(c5ccccc5-c5ccccc54)c4ccccc4-6)c4cccc5c4oc4ccccc45)c3)cc2)cc1 ; CC1(C)c2cc(N(c3ccccc3)c3ccc(-c4ccccc4)cc3)ccc2-c2ccc(N(c3ccc4c(c3)c3c5c(ccc3n4-c3ccc(-c4ccccc4)cc3)C3(c4ccccc4-c4ccccc43)c3ccccc3-5)c3cccc4c3oc3ccccc34)cc21 ; CC1(C)c2ccccc2-c2ccc(N(c3ccc4c(c3)c3c5c(ccc3n4-c3ccc(-c4ccccc4)cc3)C3(c4ccccc4-c4ccccc43)c3ccccc3-5)c3cccc4c3oc3ccccc34)cc21]

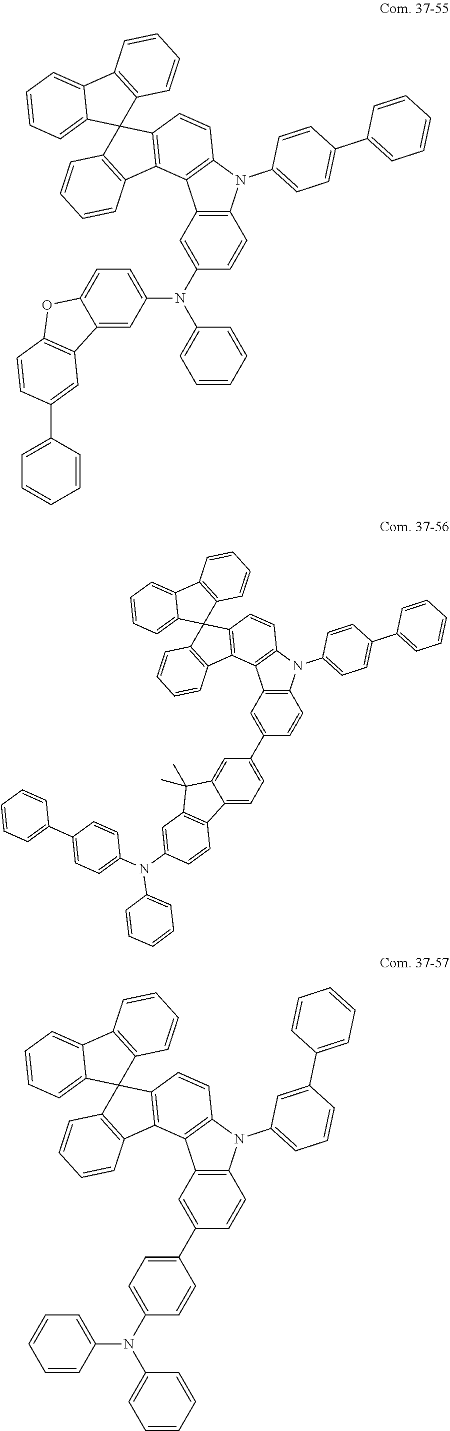

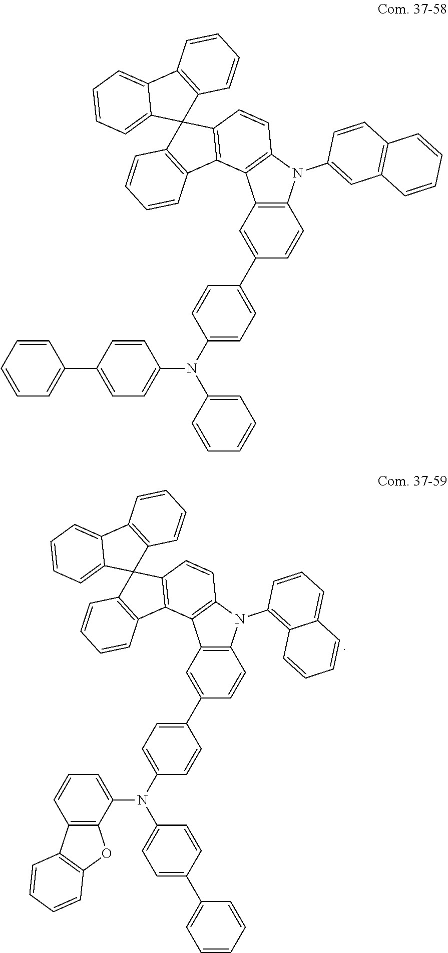 [SMILES: c1ccc(-c2ccc(N(c3ccccc3)c3ccc(-c4ccc5c(c4)c4c6c(ccc4n5-c4ccc5ccccc5c4)C4(c5ccccc5-c5ccccc54)c4ccccc4-6)cc3)cc2)cc1 ; c1ccc(-c2ccc(N(c3ccc(-c4ccc5c(c4)c4c6c(ccc4n5-c4cccc5ccccc45)C4(c5ccccc5-c5ccccc54)c4ccccc4-6)cc3)c3cccc4c3oc3ccccc34)cc2)cc1]

The spirofluorene compound according to an embodiment of the present invention may be used in an organic material layer of an organic light-emitting device due to its structural specificity, and specifically, may be used as a material for forming a hole transport layer in the organic material layer.

The compounds described above may be prepared based on Preparation Examples and Examples described below. In Preparation Examples and Examples described below, representative examples are described, and if necessary, a substituent may be added or excluded, and a position of the substituent may be changed. In addition, a starting material, a reaction material, a reaction condition, and the like may be changed based on the technology known in the art. If necessary, types or positions of substituents at remaining positions may be changed by those skilled in the art using the technology known in the art.

In addition, the present invention provides an organic light-emitting device including the spirofluorene compound of Chemical Formula 1.

Specifically, the organic light-emitting device according to the present invention includes an anode, a cathode, and one or more organic material layers provided between the anode and the cathode, and one or more layers of the organic material layers include the spirofluorene compound of Chemical Formula 1.

However, structures of the organic light-emitting device known in the art may also be applied to the present invention. The scope of the present invention is not limited by such a stacked structure.

The organic light-emitting device according to the present invention may be manufactured by materials and methods known in the art, except that the spirofluorene compound of Chemical Formula 1 is included in one or more layers of the organic material layers.

The spirofluorene compound of Chemical Formula 1 alone may constitute one or more layers of the organic material layers of the organic light-emitting device. However, if necessary, the spirofluorene compound of Chemical Formula 1 may be mixed with other materials to constitute the organic material layer.

The spirofluorene compound of Chemical Formula 1 may be used as a hole injection material, a hole transport material, a light-emitting material, an electron transport material, an electron injection material, and the like in the organic light-emitting device. The spirofluorene compound may be used as a material of one or more layers of a hole injection layer, a hole transport layer, an emissive layer, an electron transport layer, and an electron injection layer. As an example, the spirofluorene compound may be used as a material of the hole injection and transport layers of the organic light-emitting device. As an example, the spirofluorene compound may be used as a material of the electron injection and transport layers of the organic light-emitting device. In addition, as an example, the spirofluorene compound may be used as a material of the emissive layer of the organic light-emitting device. Preferably, the spirofluorene compound is used as a material of the hole transport layer of the organic light-emitting device.

In the organic light-emitting device according to the present invention, materials other than the spirofluorene compound will be described below, but these materials are for illustrative purposes only, are not intended to limit the scope of the present invention, and may be substituted by materials known in the art.

As a material of the anode, materials having a relatively high work function may be used, and as a specific example, metals such as vanadium, chromium, copper, zinc, and gold, or alloys thereof, metal oxides such as zinc oxide, indium oxide, indium tin oxide (ITO), and indium zinc oxide (IZO), a combination of a metal and an oxide, such as ZnO:Al or SnO₂:Sb, conductive polymers such as poly(3-methylthiophene), poly[3,4-(ethylene-1,2-dioxy)thiophene](PEDT), polypyrrole, and polyaniline, and the like may be used, but the material of the anode is not limited thereto. In addition, the anode layer may be formed of only one type of the materials described above or a mixture of a plurality of materials, and may be formed to have a multi-layer structure composed of a plurality of layers having the same composition or different compositions.

As a material of the cathode, materials having a relatively low work function may be used, and as a specific example, metals such as magnesium, calcium, sodium, potassium, titanium, indium, yttrium, lithium, gadolinium, aluminum, silver, tin, and lead, or alloys thereof, a multi-layered material such as LiF/Al or LiO2/Al, and the like may be used.

As the hole injection material, known hole injection materials may be used, and for example, a phthalocyanine compound such as copper phthalocyanine (CuPc) disclosed in U.S. Pat. No. 4,356,429, or a starburst-type amine derivative disclosed in the literature [Advanced Material, 6, p. 677 (1994)], for example, tris(4-carbazoyl-9-ylphenyl) amine (TCTA), 4,4′,4″-tris (3-methylphenylphenylamino)triphenylamine (m-MTDATA), 1,3,5-tris[4-(3-metylphenylphenylamino)phenyl]benzene (m-MTDAPB), polyaniline/dodecylbenzenesulfonic acid (Pani/DBSA) or poly(3,4-ethylenedioxythiophene)-poly(styrnesulfonate) (PEDOT:PSS), which is a conductive polymer having solubility, polyaniline/camphor sulfonic acid (Pani/CSA), polyaniline/poly(4-styrene-sulfonate) (PANI/PSS), or the like may be used.

As the hole transport material, the spirofluorene compound according to an embodiment of the present invention may be used alone or in combination with known hole transport materials.

Specifically, the hole transport material includes the spirofluorene compound according to an embodiment of the present invention, but may be used together with a pyrazoline derivative, an arylamine-based derivative, a stilbene derivative, a triphenyldiamine derivative, or the like, and may also be used together with a low molecular weight or high molecular weight material. Specific examples of the hole transport material include a low molecular weight hole transport material such as N,N′-bis(naphthalen-1-yl)-N,N′-bis(phenyl)-benzidine (NPB), N,N′-bis(naphthalen-1-yl)-N,N′-bis(phenyl)-2,2′-dimethylbenzidine (NPD), 1,3-bis(N-carbazolyl)benzene (mCP), N,N′-bis(3-methylphenyl)-N,N′-diphenylbenzidine (TPD), N,N,N′,N′-tetrakis(4-methylphenyl)-(1,1′-biphenyl)-4,4-diamine (TTB), N1,N4-diphenyl-N1,N4-dim-tolylbenzene-1,4-diamine (TTP), N,N′-bis(4-methylphenyl)-N,N′-bis (4-ethylphenyl)-[1,1′-(3,3′-dimethyl)biphenyl]-4,4′-diamine (ETPD), N4,N4′-di(naphthalen-1-yl)-N4,N4′-bis(4-vinylphenyl)biphenyl-4,4′-diamine (VNPB), N4,N4′-bis(4-(6-((3-ethyloxetan-3-yl)methoxy)hexyl)phenyl)-N4,N4′-diphenylbiphenyl-4,4′-diamine (ONPB), or N4,N4′-bis(4-(6-((3-ethyloxetan-3-yl)methoxy)hexyl)phenyl)-N4,N4′-diphenylbiphenyl-4,4′-diamine (OTPD); and a high molecular weight hole transport material such as poly-N-vinylcarbazole (PVK), polyaniline, or (phenylmethyl)polysilane.

As the electron transport material, an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyl dicyanoethylene and a derivative thereof, a diphenoquinone derivative, 8-hydroxyquinoline and a metal complex of derivatives thereof, or the like may be used, and a high molecular weight material as well as a low molecular weight material may also be used. As a specific example, diphenyl[4-(triphenylsilyl)phenyl]phosphine oxide (TSPO1) or 1,3,5-tris (N-phenylbenzimidazol-2-yl)benzene (TPBI); tris (8-hydroxyquinolinato)aluminum (Alq₃); 2,9-dimethyl-4,7-diphenyl-1,10-phenanthroline (BCP); an azole compound such as 2-(4-biphenyl)-5-(4-tert-butyl-phenyl)-1,3,4-oxadizole (PBD), 3-(4-biphenyl)-4-phenyl-5-(4-tert-butyl-phenyl)-1,2,4-triazole (TAZ), or 1,3-bis[2-(4-tert-butylphenyl)-1,3,4-oxadiazo-5-yl]benzene (OXD-7); tris(phenylquinoxaline) (TPQ); 3,3′-[5′-[3-(3-pyridinyl) phenyl] [1,1′:3′,1″-terphenyl]-3,3″-diyl]bispyridine (TmPyPB); and the like may be used, but the electron transport material is not limited thereto.

As the electron injection material, for example, LIF or lithium quinolate (Liq) is typically used, but the electron injection material is not limited thereto.

As the light-emitting material, a red, green, or blue light-emitting material may be used, and if necessary, a mixture of two or more light-emitting materials may be used. In addition, as the light-emitting material, a fluorescent material may be used and a phosphorescent material may also be used. As the light-emitting material, materials that emit light alone by binding holes and electrons injected from the anode and the cathode, respectively, may be used, and materials in which a host material and a dopant material are both involved in light emitting may also be used.

The emissive layer may be formed using a light-emitting material by methods such as a vacuum deposition method, a spin coating method, a cast method, and a Langmuir-Blodgett (LB) method, and more specifically, when the emissive layer is formed by a vacuum deposition method, the deposition conditions vary depending on a compound to be used, but generally may be selected within the same range of conditions as those for forming the hole injection layer. In addition, as the material of the emissive layer, a known compound may be used as a host or dopant.

In addition, as an example of the material of the emissive layer, IDE102, IDE105, BD-331, or BD-142 (N⁶,N¹²-bis (3,4-dimethylphenyl)-N⁶,N¹²-dimesitylchrysene-6,12-diamine) available from Idemitsu Kosan Co., Ltd. may be used as a fluorescent dopant, and as a phosphorescent dopant, tris(2-phenylpyridine)iridium (Ir(ppy)₃) as a green phosphorescent dopant, iridium(III) bis[4,6-difluorophenyl)-pyridinato-N,C2′]picolinate (F2Irpic) as a blue phosphorescent dopant, RD61 available from Universal Display Corporation (UDC) as a red phosphorescent dopant, and the like may be co-vacuum deposited (doped).

The phosphorescent dopant is a compound capable of emitting light from triplet excitons, and is not particularly limited as long as light is emitted from triplet excitons. As a specific example, the phosphorescent dopant may be a metal complex including one or more metals selected from the group consisting of Ir, Ru, Pd, Pt, Os, and R^{e}, and may be a porphyrin metal complex or an ortho-metallized metal complex.

The porphyrin metal complex may be specifically a porphyrin platinum complex.

The ortho-metallized metal complex may include, as ligands, 2-phenylpyridine (ppy) derivatives, 7,8-benzoquinoline derivatives, 2-(2-thienyl)pyridine (tp) derivatives, 2-(1-naphthyl)pyridine (npy) derivatives, 2-phenylquinoline (pq) derivatives, and the like. In this case, these derivatives may have substituents, if necessary. The ortho-metallized metal complex may further have ligands other than the above ligands, such as acetylacetonato (acac) and picric acid, as auxiliary ligands. Specific examples of the ortho-metallized metal complex include, but are not limited to, bisthienylpyridine acetylacetonate iridium, bis(2-benzo[b]thiophen-2-yl-pyridine) (acetylacetonato)iridium(III) (Ir(btp)₂(acac)), bis(2-phenylbenzothiazole) (acetylacetonato)iridium(III) (Ir(bt)₂(acac)), bis(1-phenylisoquinoline) (acetylacetonato)iridium(III) (Ir(piq)₂(acac)), tris(1-phenylisoquinoline)iridium(III) (Ir(piq)₃), tris(2-phenylpyridine)iridium(III) (Ir(ppy)₃), tris(2-biphenylpyridine)iridium, tris(3-biphenylpyridine)iridium, and tris(4-biphenylpyridine)iridium.

In addition, when the phosphorescent dopant is also used in the emissive layer, a hole blocking layer (HBL) may be additionally stacked by a vacuum deposition method or a spin coating material to prevent diffusion of triplet excitons or holes into the electron transport layer. In this case, the hole blocking material that may be used is not particularly limited, and any known hole blocking material may be selected and used. For example, the hole blocking material may be an oxadiazole derivative, a triazole derivative, or a phenanthroline derivative, and specifically, bis(8-hydroxy-2-methylquinolinato)-aluminum biphenoxide (Balq), a phenanthroline-based compound (Universal Display Corporation (UDC), bathocuproine (BCP)), and the like may be used.

Hereinafter, the present invention will be described in more detail with reference to Examples, but these Examples are only for exemplifying the present invention and are not intended to limit the scope of the present invention.

### [Preparation Example 1] Preparation of Compound A-1

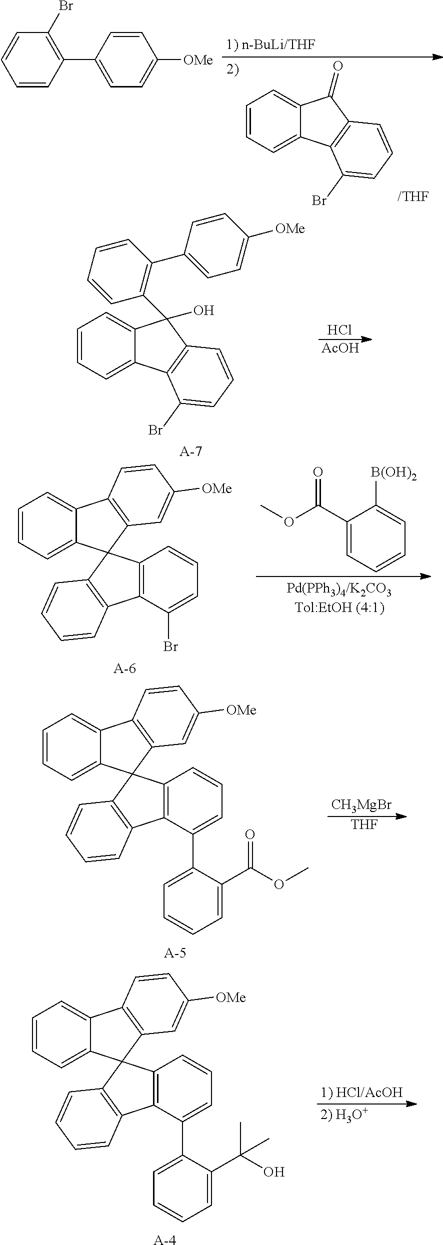

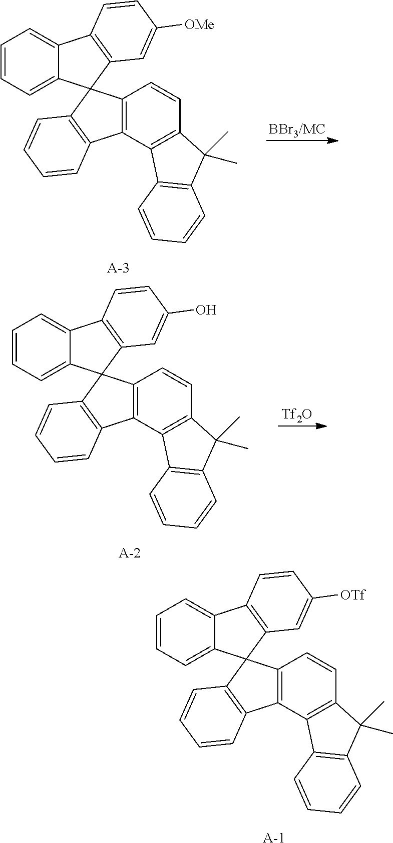

Preparation of Compound A-7

10 g of (38.00 mmol) of 2-bromo-4′-methoxy-1,1′-biphenyl was dissolved in 200 mL of tetrahydrofuran (THF), the temperature was lowered to −78° C., 29 mL (45.61 mmol) of n-butyllithium (1.6 M solution in hexane) was slowly added dropwise thereto, and stirring was performed at the same temperature for 30 minutes. While stirring was continued, a 4-bromo-9H-fluoren-9-one solution (solution obtained by dissolving 10.83 g (41.80 mmol) of 4-bromo-9H-fluoren-9-one in 210 mL of THF) was slowly added dropwise at the same temperature. After dropwise addition was completed, the temperature was slowly raised to room temperature, and the reaction was stopped. Filtering was performed using celite and florisil, and then washing was performed with methylene chloride (MC). Purification was performed by column chromatography using MC and Hex (hexane), thereby obtaining 12.13 g (72%) of a target compound A-7.

Preparation of Compound A-6

10 g (22.57 mmol) of the compound A-7 was dissolved in 120 mL of AcOH, 50 mL of HCl was added thereto, and stirring was performed under reflux at 130° C. for 24 hours. After the reaction was stopped, the reaction solution was cooled to room temperature, and the produced solid was filtered and washed with Hex and ethyl acetate (EA). Thereafter, recrystallization was performed with Hex/EA, thereby obtaining 7.10 g (74%) of a target compound A-6.

Preparation of Compound A-5

10 g (23.51 mmol) of the compound A-6 was dissolved in 100 mL of toluene, 5.08 g (28.21 mmol) of (2-(methoxycarbonyl)phenyl)boronic acid, 1.36 g (1.18 mmol) of tetrakis (triphenylphosphine)palladium(0) (Pd(PPh₃)₄), and 8.12 g (58.78 mmol) of potassium carbonate (K₂CO₃) were added, and stirring was performed. 25 mL of ethanol and 25 mL of water were added thereto, and the mixed solution was refluxed at 130° C. for 24 hours. After the reaction was completed, extraction was performed using MC and H₂O, the solvent was removed, and then purification was performed by column chromatography with Hex and MC, thereby obtaining 8.59 g (76%) of a target compound A-5.

Preparation of Compound A-4

10 g (20.81 mmol) of the compound A-5 was dissolved in 110 mL of THF, and then the temperature was lowered to −78° C. 16 mL (47.86 mmol) of methylmagnesium bromide (CH₃MgBr, 3.0 M solution in diethyl ether) was slowly added dropwise thereto, and stirring was performed at the same temperature for 30 minutes. After stirring was completed, the temperature of the reaction solution was slowly raised to room temperature, and stirring was additionally performed at room temperature for 24 hours. After the reaction was completed, extraction was performed using a sodium thiosulfate (Na₂S2O₃) solution and MC, the solvent was removed, and recrystallization purification was performed with Hex and EA, thereby obtaining 7.00 g (70%) of a target compound A-4.

Preparation of Compound A-3

10 g (20.81 mmol) of the compound A-4 was dissolved in 120 mL of AcOH, 50 mL of HCl was added thereto, and stirring was performed under reflux at 130° C. for 24 hours. After the reaction was completed, the reaction solution was cooled to room temperature, the produced solid was filtered, and washing was performed with a sodium bicarbonate (NaHCO₃) aqueous solution. Thereafter, washing was performed with water, ethanol, Hex, and EA, and then recrystallization was performed with Hex/EA, thereby obtaining 7.90 g (82%) of a target compound A-3.

Preparation of Compound A-2

10 g (21.62 mmol) of the compound A-3 was dissolved in 110 mL of MC, and then the temperature was lowered to 0° C. 25 mL (25.94 mmol) of tribromoboron (1.0 M solution in MC) was slowly added dropwise thereto, and stirring was performed at the same temperature for 30 minutes. The temperature of the reaction solution was slowly raised to room temperature, and stirring was additionally performed at room temperature for 24 hours. After the reaction was completed, extraction was performed with MC and a NaHCO₃ aqueous solution, the MC layer was collected, the solvent was removed, and the produced solid was purified by column chromatography using MC and Hex, thereby obtaining 6.59 g (68%) of a target compound A-2.

Preparation of Compound A-1

10 g (22.30 mmol) of the compound A-2 was dissolved in 120 mL of THF, 3.1 mL (22.30 mmol) of triethylamine (TEA) and 4.5 mL (26.76 mmol) of triflic anhydride (CF₃SO₂)₂O) were added, and then stirring was performed at room temperature for 24 hours. After the reaction was completed, extraction was performed using MC and a NaHCO₃ aqueous solution, the MC layer was collected, the solvent was removed, and the produced solid was purified by column chromatography using MC and Hex, thereby obtaining 8.93 g (69%) of a target compound A-1.

### [Example 1] Preparation of Compounds P1 and P2

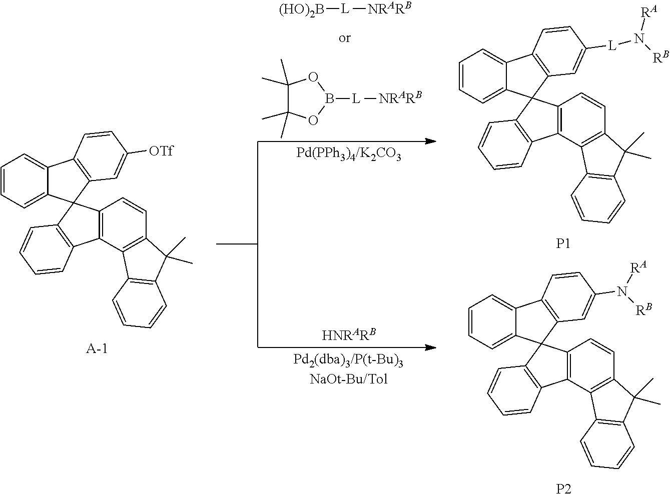

Preparation of Compound P1

A target compound P1 was obtained through a Suzuki coupling reaction using the compound A-1, a substituted boronic acid or a boronic ester compound, and a palladium catalyst.

Preparation of Compound P2

A target compound P2 was obtained through palladium-amination using the compound A-1, a secondary amine compound, and a palladium catalyst.

The structures of the substituted boronic acid, boronic ester, and secondary amine compound as the reactants and the structures and yields of the prepared compounds P1 and P2 are shown in Table 1.

TABLE 1

| com- | | | |
|---|---|---|---|
| pound | | | |
| No. | Reactant | Product (P1, P2) | Yield |
| Com 21-1 | 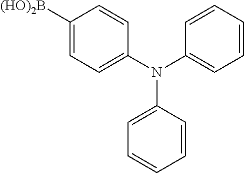 [SMILES: OB(O)c1ccc(N(c2ccccc2)c2ccccc2)cc1] | 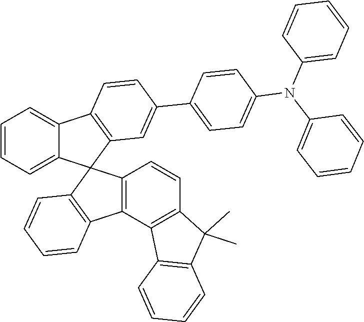 [SMILES: CC1(C)c2ccccc2-c2c1ccc1c2-c2ccccc2C12c1ccccc1-c1ccc(-c3ccc(N(c4ccccc4)c4ccccc4)cc3)cc12] | 76% |
| Com 21-2 | 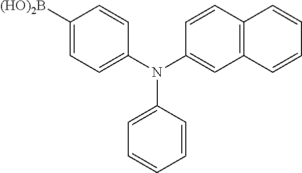 [SMILES: OB(O)c1ccc(N(c2ccccc2)c2ccc3ccccc3c2)cc1] | 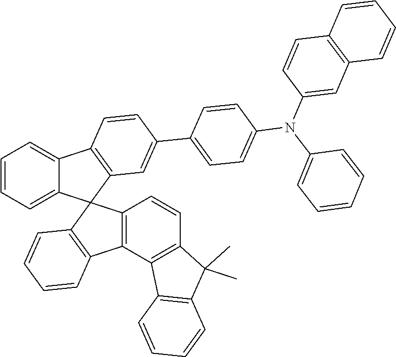 [SMILES: CC1(C)c2ccccc2-c2c1ccc1c2-c2ccccc2C12c1ccccc1-c1ccc(-c3ccc(N(c4ccccc4)c4ccc5ccccc5c4)cc3)cc12] | 80% |
| Com 21-3 | 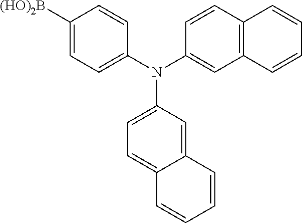 [SMILES: OB(O)c1ccc(N(c2ccc3ccccc3c2)c2ccc3ccccc3c2)cc1] | 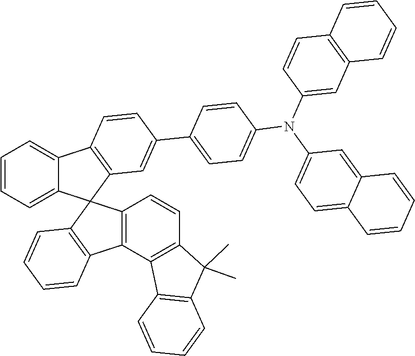 [SMILES: CC1(C)c2ccccc2-c2c1ccc1c2-c2ccccc2C12c1ccccc1-c1ccc(-c3ccc(N(c4ccc5ccccc5c4)c4ccc5ccccc5c4)cc3)cc12] | 71% |
| Com 21-4 | 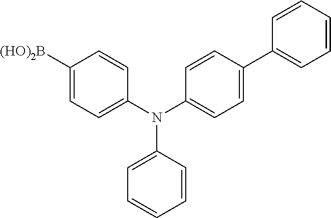 [SMILES: OB(O)c1ccc(N(c2ccccc2)c2ccc(-c3ccccc3)cc2)cc1] | 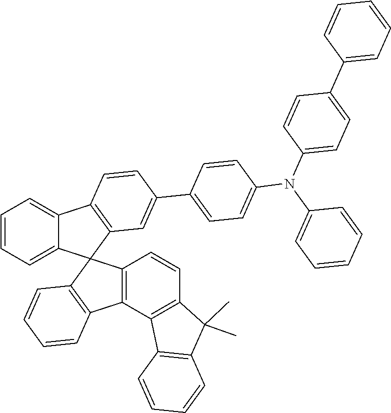 [SMILES: CC1(C)c2ccccc2-c2c1ccc1c2-c2ccccc2C12c1ccccc1-c1ccc(-c3ccc(N(c4ccccc4)c4ccc(-c5ccccc5)cc4)cc3)cc12] | 75% |
| Com 21-5 | 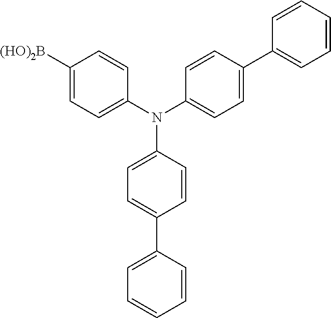 [SMILES: OB(O)c1ccc(N(c2ccc(-c3ccccc3)cc2)c2ccc(-c3ccccc3)cc2)cc1] | 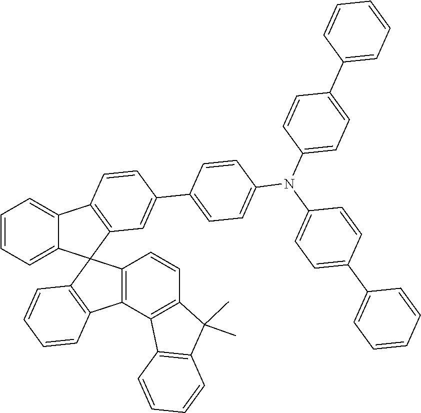 [SMILES: CC1(C)c2ccccc2-c2c1ccc1c2-c2ccccc2C12c1ccccc1-c1ccc(-c3ccc(N(c4ccc(-c5ccccc5)cc4)c4ccc(-c5ccccc5)cc4)cc3)cc12] | 75% |
| Com 21-6 | 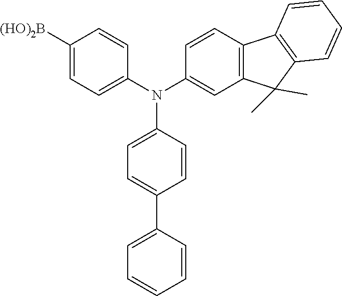 [SMILES: CC1(C)c2ccccc2-c2ccc(N(c3ccc(B(O)O)cc3)c3ccc(-c4ccccc4)cc3)cc21] | 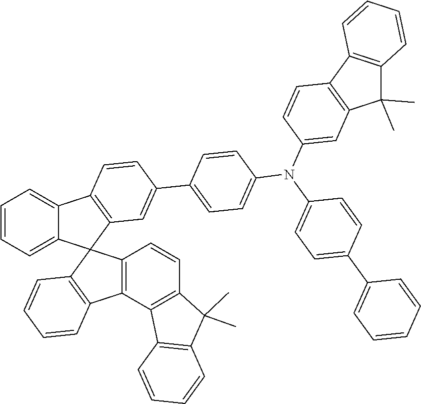 [SMILES: CC1(C)c2ccccc2-c2ccc(N(c3ccc(-c4ccccc4)cc3)c3ccc(-c4ccc5c(c4)C4(c6ccccc6-5)c5ccccc5-c5c4ccc4c5-c5ccccc5C4(C)C)cc3)cc21] | 69% |
| Com 21-7 | 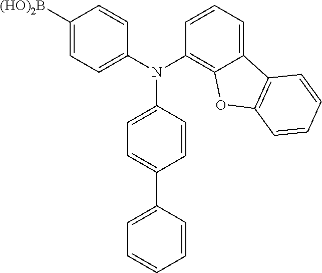 [SMILES: OB(O)c1ccc(N(c2ccc(-c3ccccc3)cc2)c2cccc3c2oc2ccccc23)cc1] | 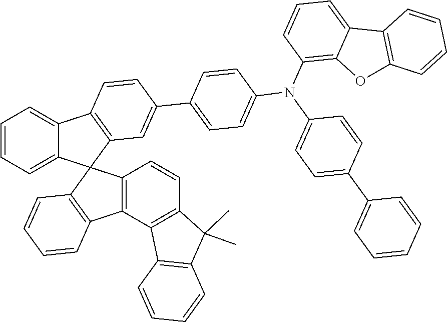 [SMILES: CC1(C)c2ccccc2-c2c1ccc1c2-c2ccccc2C12c1ccccc1-c1ccc(-c3ccc(N(c4ccc(-c5ccccc5)cc4)c4cccc5c4oc4ccccc45)cc3)cc12] | 65% |
| Com 21-8 | 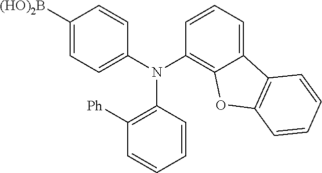 [SMILES: OB(O)c1ccc(N(c2ccccc2-c2ccccc2)c2cccc3c2oc2ccccc23)cc1] | 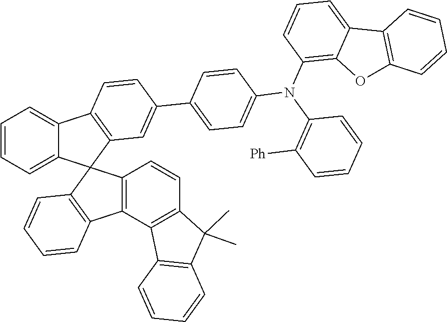 [SMILES: CC1(C)c2ccccc2-c2c1ccc1c2-c2ccccc2C12c1ccccc1-c1ccc(-c3ccc(N(c4ccccc4-c4ccccc4)c4cccc5c4oc4ccccc45)cc3)cc12] | 63% |
| Com 21-9 | 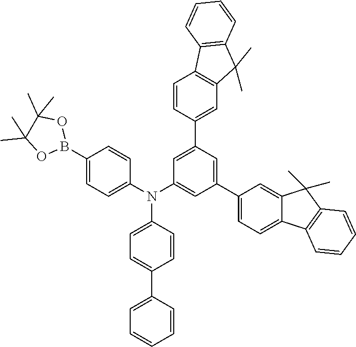 [SMILES: CC1(C)c2ccccc2-c2ccc(-c3cc(-c4ccc5c(c4)C(C)(C)c4ccccc4-5)cc(N(c4ccc(B5OC(C)(C)C(C)(C)O5)cc4)c4ccc(-c5ccccc5)cc4)c3)cc21] | 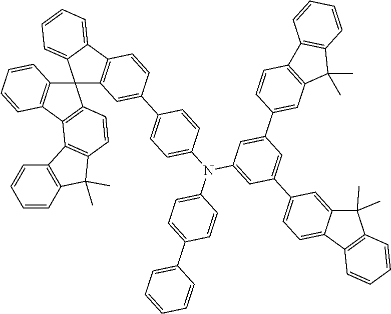 [SMILES: CC1(C)c2ccccc2-c2ccc(-c3cc(-c4ccc5c(c4)C(C)(C)c4ccccc4-5)cc(N(c4ccc(-c5ccccc5)cc4)c4ccc(-c5ccc6c(c5)C5(c7ccccc7-6)c6ccccc6-c6c5ccc5c6-c6ccccc6C5(C)C)cc4)c3)cc21] | |
| Com 21-10 | 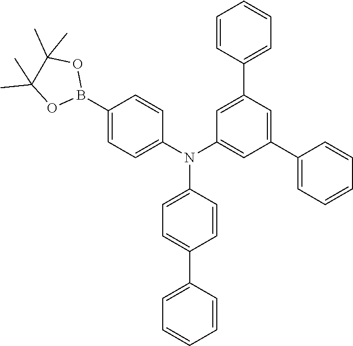 [SMILES: CC1(C)OB(c2ccc(N(c3ccc(-c4ccccc4)cc3)c3cc(-c4ccccc4)cc(-c4ccccc4)c3)cc2)OC1(C)C] | 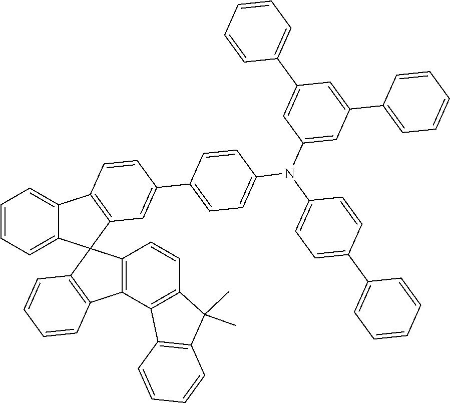 [SMILES: CC1(C)c2ccccc2-c2c1ccc1c2-c2ccccc2C12c1ccccc1-c1ccc(-c3ccc(N(c4ccc(-c5ccccc5)cc4)c4cc(-c5ccccc5)cc(-c5ccccc5)c4)cc3)cc12] | 75% |
| Com 21-11 | 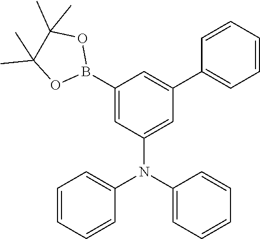 [SMILES: CC1(C)OB(c2cc(-c3ccccc3)cc(N(c3ccccc3)c3ccccc3)c2)OC1(C)C] | 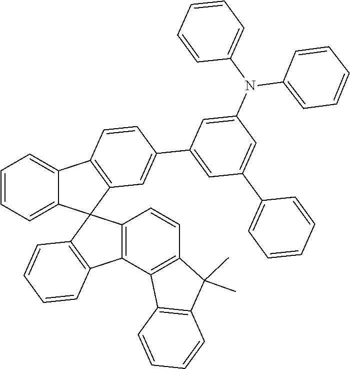 [SMILES: CC1(C)c2ccccc2-c2c1ccc1c2-c2ccccc2C12c1ccccc1-c1ccc(-c3cc(-c4ccccc4)cc(N(c4ccccc4)c4ccccc4)c3)cc12] | 69% |
| Com 21-12 | 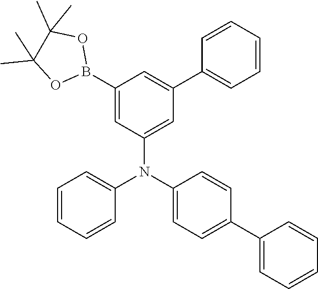 [SMILES: CC1(C)OB(c2cc(-c3ccccc3)cc(N(c3ccccc3)c3ccc(-c4ccccc4)cc3)c2)OC1(C)C] | 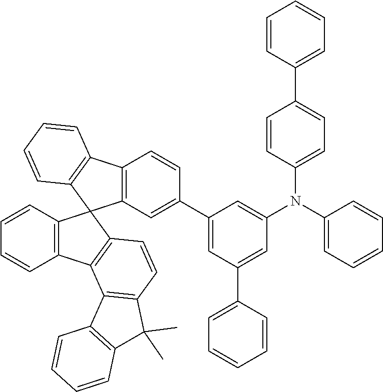 [SMILES: CC1(C)c2ccccc2-c2c1ccc1c2-c2ccccc2C12c1ccccc1-c1ccc(-c3cc(-c4ccccc4)cc(N(c4ccccc4)c4ccc(-c5ccccc5)cc4)c3)cc12] | 65% |
| Com 21-13 | 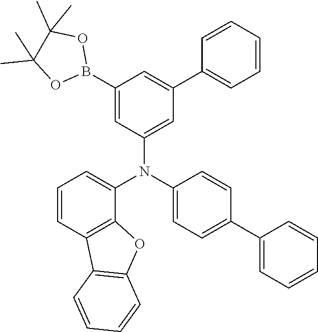 [SMILES: CC1(C)OB(c2cc(-c3ccccc3)cc(N(c3ccc(-c4ccccc4)cc3)c3cccc4c3oc3ccccc34)c2)OC1(C)C] | 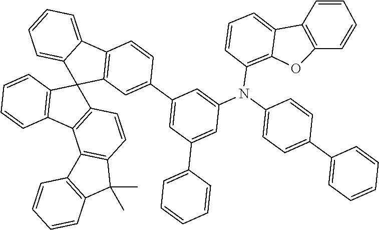 [SMILES: CC1(C)c2ccccc2-c2c1ccc1c2-c2ccccc2C12c1ccccc1-c1ccc(-c3cc(-c4ccccc4)cc(N(c4ccc(-c5ccccc5)cc4)c4cccc5c4oc4ccccc45)c3)cc12] | 63% |
| Com 21-14 | 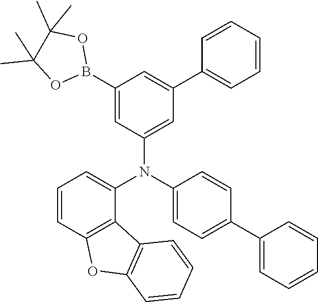 [SMILES: CC1(C)OB(c2cc(-c3ccccc3)cc(N(c3ccc(-c4ccccc4)cc3)c3cccc4oc5ccccc5c34)c2)OC1(C)C] | 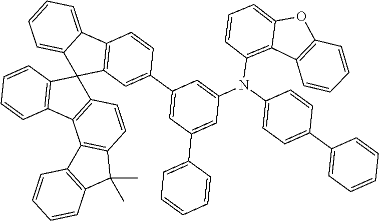 [SMILES: CC1(C)c2ccccc2-c2c1ccc1c2-c2ccccc2C12c1ccccc1-c1ccc(-c3cc(-c4ccccc4)cc(N(c4ccc(-c5ccccc5)cc4)c4cccc5oc6ccccc6c45)c3)cc12] | 71% |
| Com 21-15 | 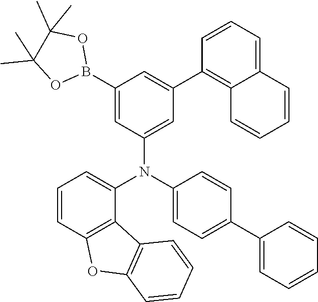 [SMILES: CC1(C)OB(c2cc(-c3cccc4ccccc34)cc(N(c3ccc(-c4ccccc4)cc3)c3cccc4oc5ccccc5c34)c2)OC1(C)C] | 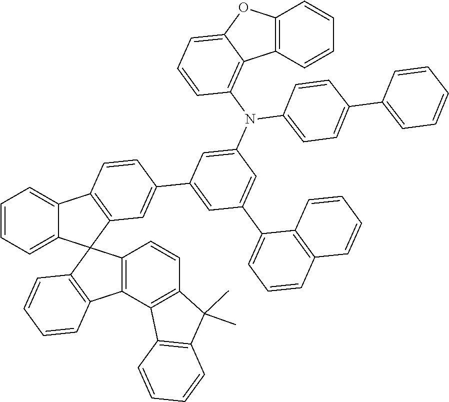 [SMILES: CC1(C)c2ccccc2-c2c1ccc1c2-c2ccccc2C12c1ccccc1-c1ccc(-c3cc(-c4cccc5ccccc45)cc(N(c4ccc(-c5ccccc5)cc4)c4cccc5oc6ccccc6c45)c3)cc12] | 58% |
| Com 21-16 | 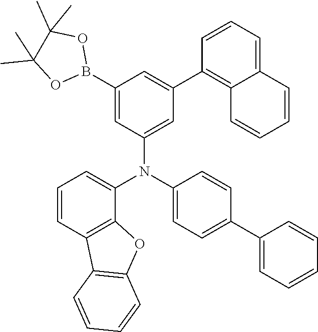 [SMILES: CC1(C)OB(c2cc(-c3cccc4ccccc34)cc(N(c3ccc(-c4ccccc4)cc3)c3cccc4c3oc3ccccc34)c2)OC1(C)C] | 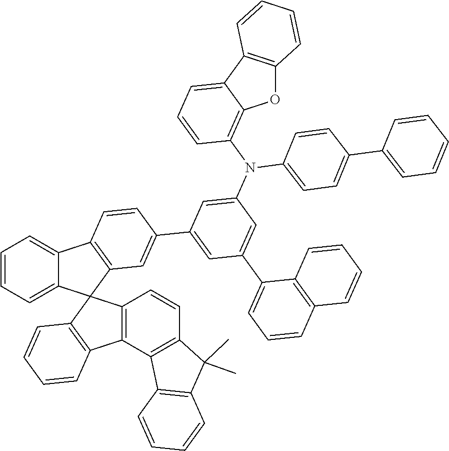 [SMILES: CC1(C)c2ccccc2-c2c1ccc1c2-c2ccccc2C12c1ccccc1-c1ccc(-c3cc(-c4cccc5ccccc45)cc(N(c4ccc(-c5ccccc5)cc4)c4cccc5c4oc4ccccc45)c3)cc12] | 61% |
| Com 21-17 | 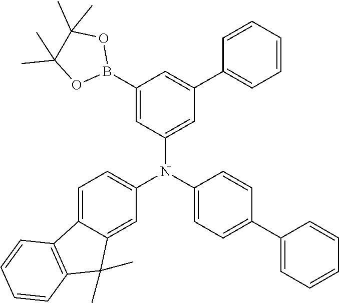 [SMILES: CC1(C)c2ccccc2-c2ccc(N(c3ccc(-c4ccccc4)cc3)c3cc(B4OC(C)(C)C(C)(C)O4)cc(-c4ccccc4)c3)cc21] | 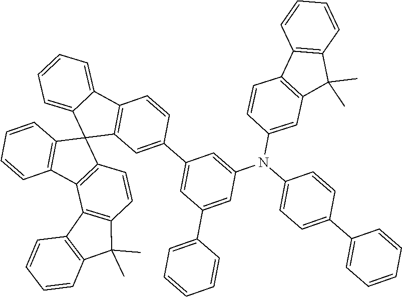 [SMILES: CC1(C)c2ccccc2-c2ccc(N(c3ccc(-c4ccccc4)cc3)c3cc(-c4ccccc4)cc(-c4ccc5c(c4)C4(c6ccccc6-5)c5ccccc5-c5c4ccc4c5-c5ccccc5C4(C)C)c3)cc21] | 69% |
| Com 21-18 | 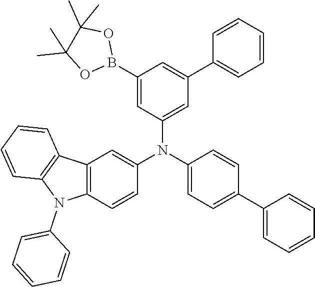 [SMILES: CC1(C)OB(c2cc(-c3ccccc3)cc(N(c3ccc(-c4ccccc4)cc3)c3ccc4c(c3)c3ccccc3n4-c3ccccc3)c2)OC1(C)C] | 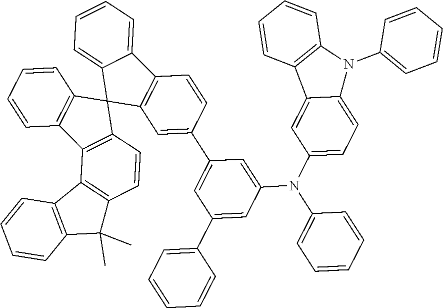 [SMILES: CC1(C)c2ccccc2-c2c1ccc1c2-c2ccccc2C12c1ccccc1-c1ccc(-c3cc(-c4ccccc4)cc(N(c4ccccc4)c4ccc5c(c4)c4ccccc4n5-c4ccccc4)c3)cc12] | 74% |
| Com 21-19 | 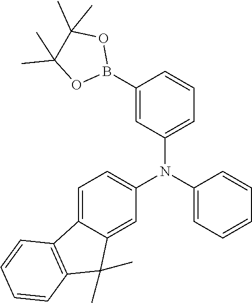 [SMILES: CC1(C)c2ccccc2-c2ccc(N(c3ccccc3)c3cccc(B4OC(C)(C)C(C)(C)O4)c3)cc21] | 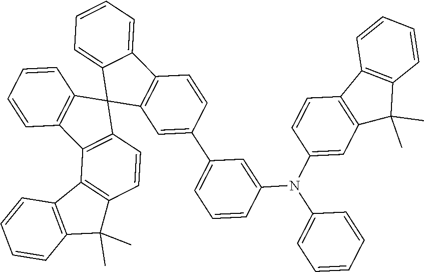 [SMILES: CC1(C)c2ccccc2-c2ccc(N(c3ccccc3)c3cccc(-c4ccc5c(c4)C4(c6ccccc6-5)c5ccccc5-c5c4ccc4c5-c5ccccc5C4(C)C)c3)cc21] | 87% |
| Com 21-20 | 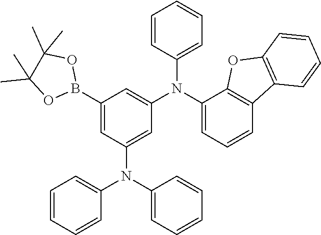 [SMILES: CC1(C)OB(c2cc(N(c3ccccc3)c3ccccc3)cc(N(c3ccccc3)c3cccc4c3oc3ccccc34)c2)OC1(C)C] | 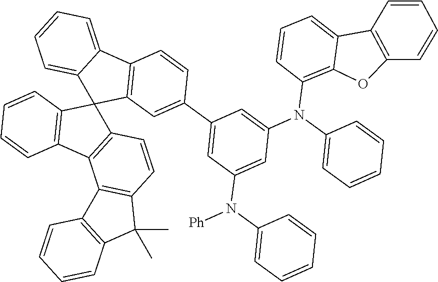 [SMILES: CC1(C)c2ccccc2-c2c1ccc1c2-c2ccccc2C12c1ccccc1-c1ccc(-c3cc(N(c4ccccc4)c4ccccc4)cc(N(c4ccccc4)c4cccc5c4oc4ccccc45)c3)cc12] | 81% |
| Com 21-21 | 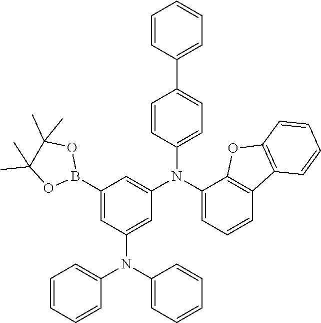 [SMILES: CC1(C)OB(c2cc(N(c3ccccc3)c3ccccc3)cc(N(c3ccc(-c4ccccc4)cc3)c3cccc4c3oc3ccccc34)c2)OC1(C)C] | 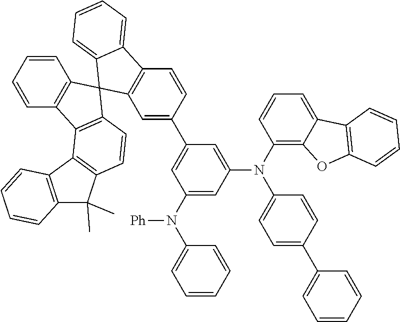 [SMILES: CC1(C)c2ccccc2-c2c1ccc1c2-c2ccccc2C12c1ccccc1-c1ccc(-c3cc(N(c4ccccc4)c4ccccc4)cc(N(c4ccc(-c5ccccc5)cc4)c4cccc5c4oc4ccccc45)c3)cc12] | 75% |
| Com 21-22 | 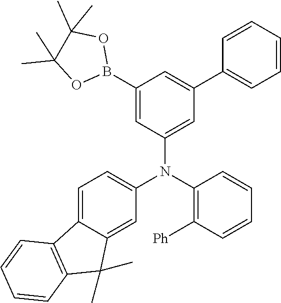 [SMILES: CC1(C)c2ccccc2-c2ccc(N(c3cc(B4OC(C)(C)C(C)(C)O4)cc(-c4ccccc4)c3)c3ccccc3-c3ccccc3)cc21] | 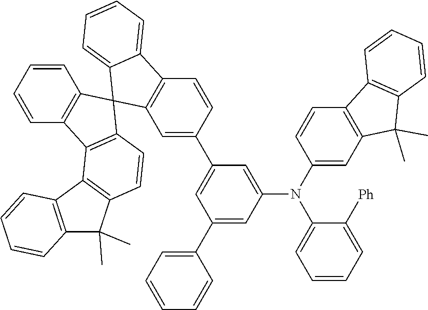 [SMILES: CC1(C)c2ccccc2-c2ccc(N(c3cc(-c4ccccc4)cc(-c4ccc5c(c4)C4(c6ccccc6-5)c5ccccc5-c5c4ccc4c5-c5ccccc5C4(C)C)c3)c3ccccc3-c3ccccc3)cc21] | 80% |
| Com 21-23 | 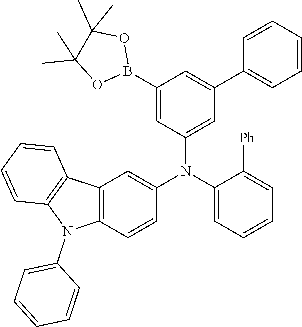 [SMILES: CC1(C)OB(c2cc(-c3ccccc3)cc(N(c3ccc4c(c3)c3ccccc3n4-c3ccccc3)c3ccccc3-c3ccccc3)c2)OC1(C)C] | 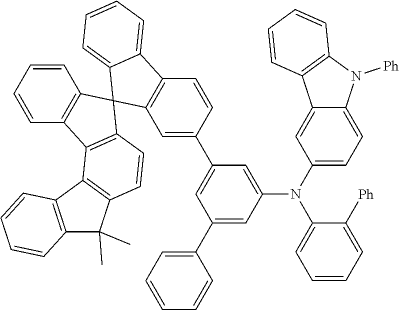 [SMILES: CC1(C)c2ccccc2-c2c1ccc1c2-c2ccccc2C12c1ccccc1-c1ccc(-c3cc(-c4ccccc4)cc(N(c4ccc5c(c4)c4ccccc4n5-c4ccccc4)c4ccccc4-c4ccccc4)c3)cc12] | 62% |
| Com 21-24 | 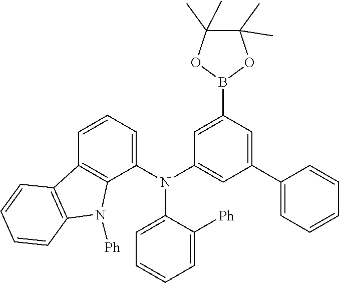 [SMILES: CC1(C)OB(c2cc(-c3ccccc3)cc(N(c3ccccc3-c3ccccc3)c3cccc4c5ccccc5n(-c5ccccc5)c34)c2)OC1(C)C] | 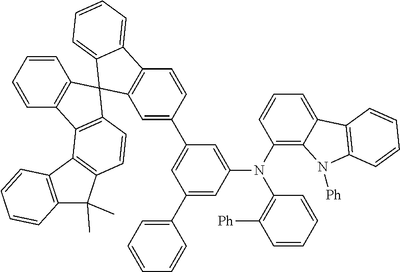 [SMILES: CC1(C)c2ccccc2-c2c1ccc1c2-c2ccccc2C12c1ccccc1-c1ccc(-c3cc(-c4ccccc4)cc(N(c4ccccc4-c4ccccc4)c4cccc5c6ccccc6n(-c6ccccc6)c45)c3)cc12] | 61% |
| Com 21-25 | 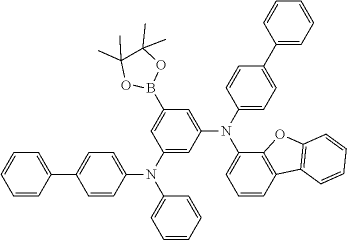 [SMILES: CC1(C)OB(c2cc(N(c3ccccc3)c3ccc(-c4ccccc4)cc3)cc(N(c3ccc(-c4ccccc4)cc3)c3cccc4c3oc3ccccc34)c2)OC1(C)C] | 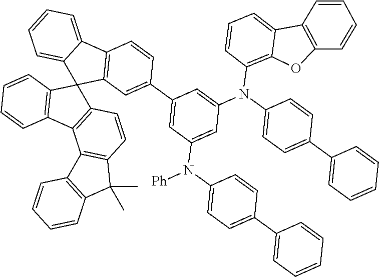 [SMILES: CC1(C)c2ccccc2-c2c1ccc1c2-c2ccccc2C12c1ccccc1-c1ccc(-c3cc(N(c4ccccc4)c4ccc(-c5ccccc5)cc4)cc(N(c4ccc(-c5ccccc5)cc4)c4cccc5c4oc4ccccc45)c3)cc12] | 64% |
| Com 21-26 | 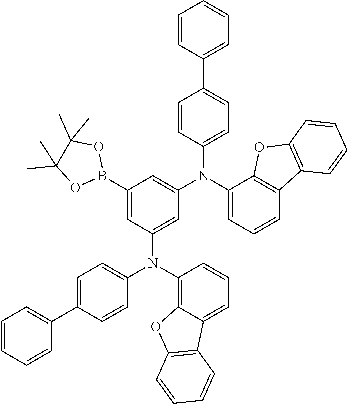 [SMILES: CC1(C)OB(c2cc(N(c3ccc(-c4ccccc4)cc3)c3cccc4c3oc3ccccc34)cc(N(c3ccc(-c4ccccc4)cc3)c3cccc4c3oc3ccccc34)c2)OC1(C)C] | 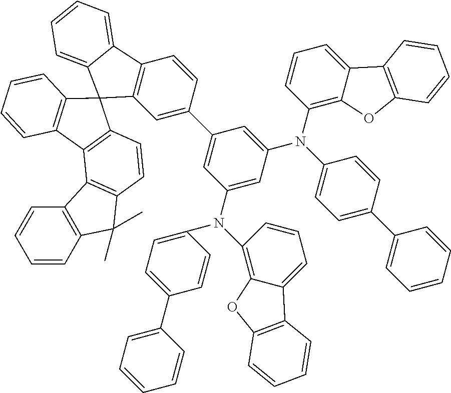 [SMILES: CC1(C)c2ccccc2-c2c1ccc1c2-c2ccccc2C12c1ccccc1-c1ccc(-c3cc(N(c4ccc(-c5ccccc5)cc4)c4cccc5c4oc4ccccc45)cc(N(c4ccc(-c5ccccc5)cc4)c4cccc5c4oc4ccccc45)c3)cc12] | 54% |
| Com 21-27 | 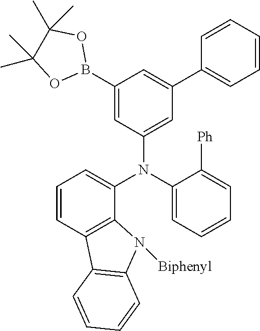 | 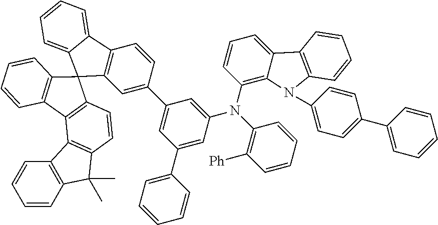 [SMILES: CC1(C)c2ccccc2-c2c1ccc1c2-c2ccccc2C12c1ccccc1-c1ccc(-c3cc(-c4ccccc4)cc(N(c4ccccc4-c4ccccc4)c4cccc5c6ccccc6n(-c6ccc(-c7ccccc7)cc6)c45)c3)cc12] | 65% |
| Com 21-28 | 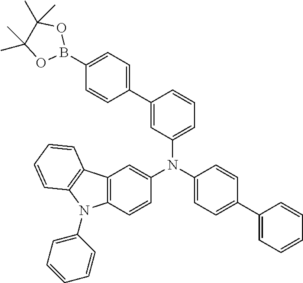 [SMILES: CC1(C)OB(c2ccc(-c3cccc(N(c4ccc(-c5ccccc5)cc4)c4ccc5c(c4)c4ccccc4n5-c4ccccc4)c3)cc2)OC1(C)C] | 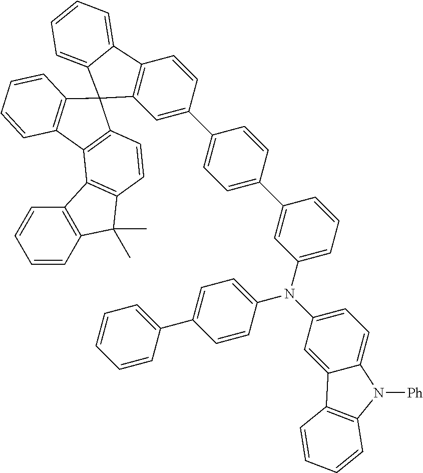 [SMILES: CC1(C)c2ccccc2-c2c1ccc1c2-c2ccccc2C12c1ccccc1-c1ccc(-c3ccc(-c4cccc(N(c5ccc(-c6ccccc6)cc5)c5ccc6c(c5)c5ccccc5n6-c5ccccc5)c4)cc3)cc12] | 78% |
| Com 21-29 | 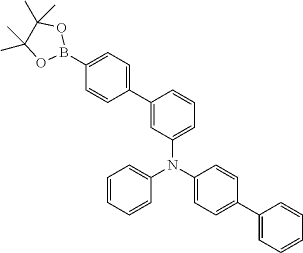 [SMILES: CC1(C)OB(c2ccc(-c3cccc(N(c4ccccc4)c4ccc(-c5ccccc5)cc4)c3)cc2)OC1(C)C] | 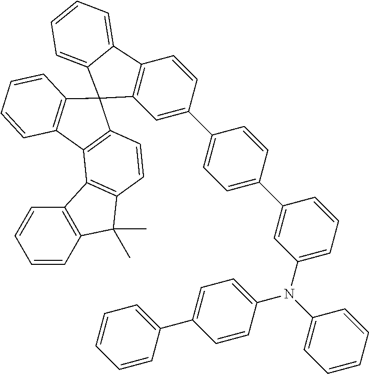 [SMILES: CC1(C)c2ccccc2-c2c1ccc1c2-c2ccccc2C12c1ccccc1-c1ccc(-c3ccc(-c4cccc(N(c5ccccc5)c5ccc(-c6ccccc6)cc5)c4)cc3)cc12] | 80% |
| Com 21-30 | 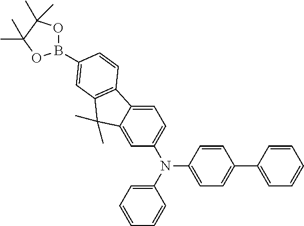 [SMILES: CC1(C)c2cc(B3OC(C)(C)C(C)(C)O3)ccc2-c2ccc(N(c3ccccc3)c3ccc(-c4ccccc4)cc3)cc21] | 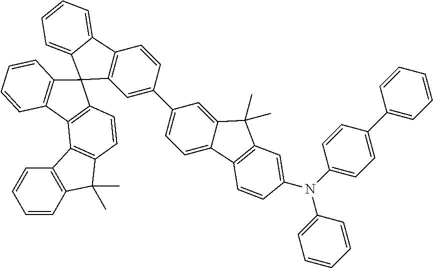 [SMILES: CC1(C)c2cc(-c3ccc4c(c3)C3(c5ccccc5-4)c4ccccc4-c4c3ccc3c4-c4ccccc4C3(C)C)ccc2-c2ccc(N(c3ccccc3)c3ccc(-c4ccccc4)cc3)cc21] | 89% |
| Com 21-31 | 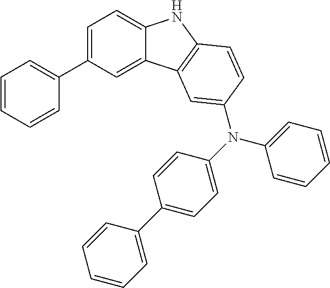 [SMILES: c1ccc(-c2ccc(N(c3ccccc3)c3ccc4[nH]c5ccc(-c6ccccc6)cc5c4c3)cc2)cc1] | 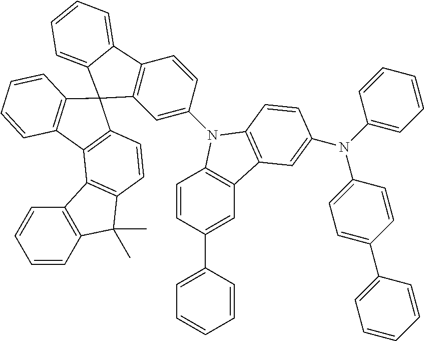 [SMILES: CC1(C)c2ccccc2-c2c1ccc1c2-c2ccccc2C12c1ccccc1-c1ccc(-n3c4ccc(-c5ccccc5)cc4c4cc(N(c5ccccc5)c5ccc(-c6ccccc6)cc5)ccc43)cc12] | 91% |
| Com 21-32 | 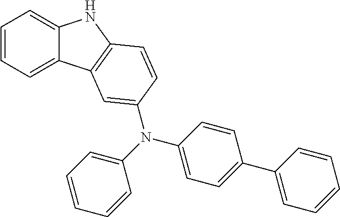 [SMILES: c1ccc(-c2ccc(N(c3ccccc3)c3ccc4[nH]c5ccccc5c4c3)cc2)cc1] | 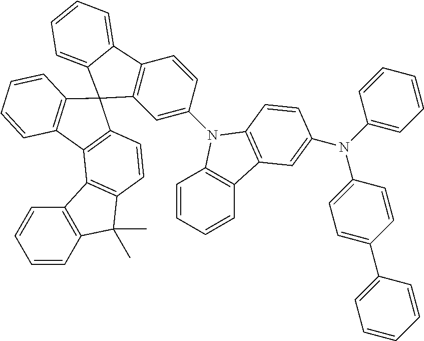 [SMILES: CC1(C)c2ccccc2-c2c1ccc1c2-c2ccccc2C12c1ccccc1-c1ccc(-n3c4ccccc4c4cc(N(c5ccccc5)c5ccc(-c6ccccc6)cc5)ccc43)cc12] | 88% |
| Com 21-33 | 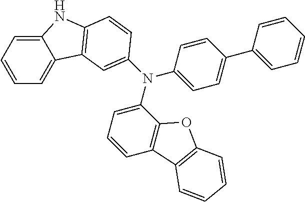 [SMILES: c1ccc(-c2ccc(N(c3ccc4[nH]c5ccccc5c4c3)c3cccc4c3oc3ccccc34)cc2)cc1] | 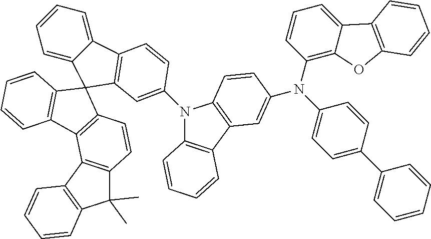 [SMILES: CC1(C)c2ccccc2-c2c1ccc1c2-c2ccccc2C12c1ccccc1-c1ccc(-n3c4ccccc4c4cc(N(c5ccc(-c6ccccc6)cc5)c5cccc6c5oc5ccccc56)ccc43)cc12] | 74% |
| Com 21-34 | 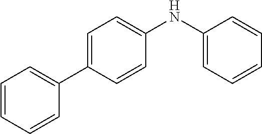 [SMILES: c1ccc(Nc2ccc(-c3ccccc3)cc2)cc1] | 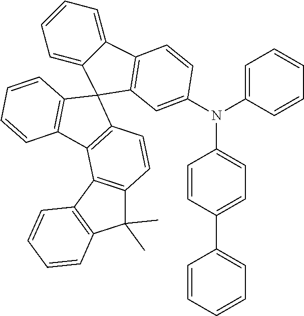 [SMILES: CC1(C)c2ccccc2-c2c1ccc1c2-c2ccccc2C12c1ccccc1-c1ccc(N(c3ccccc3)c3ccc(-c4ccccc4)cc3)cc12] | 92% |
| Com 21-35 | 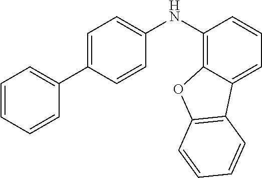 [SMILES: c1ccc(-c2ccc(Nc3cccc4c3oc3ccccc34)cc2)cc1] | 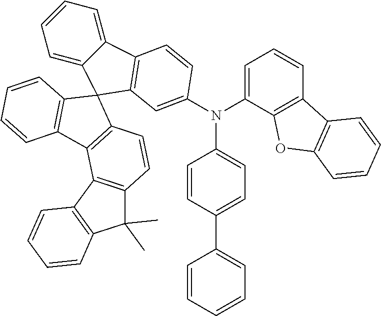 [SMILES: CC1(C)c2ccccc2-c2c1ccc1c2-c2ccccc2C12c1ccccc1-c1ccc(N(c3ccc(-c4ccccc4)cc3)c3cccc4c3oc3ccccc34)cc12] | 94% |
| Com 21-36 | 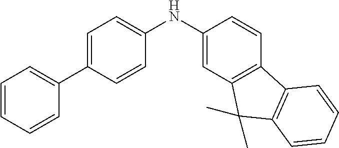 [SMILES: CC1(C)c2ccccc2-c2ccc(Nc3ccc(-c4ccccc4)cc3)cc21] | 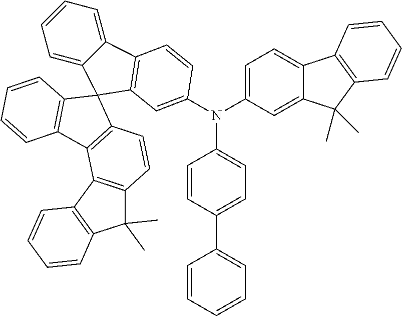 [SMILES: CC1(C)c2ccccc2-c2ccc(N(c3ccc(-c4ccccc4)cc3)c3ccc4c(c3)C3(c5ccccc5-4)c4ccccc4-c4c3ccc3c4-c4ccccc4C3(C)C)cc21] | 93% |
| Com 21-37 | 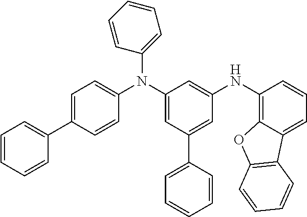 [SMILES: c1ccc(-c2ccc(N(c3ccccc3)c3cc(Nc4cccc5c4oc4ccccc45)cc(-c4ccccc4)c3)cc2)cc1] | 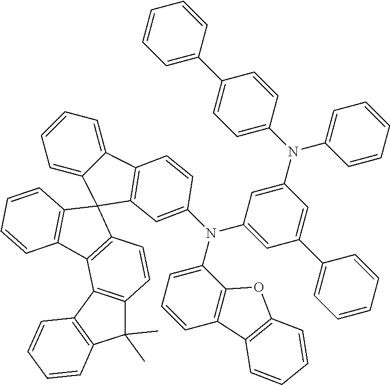 [SMILES: CC1(C)c2ccccc2-c2c1ccc1c2-c2ccccc2C12c1ccccc1-c1ccc(N(c3cc(-c4ccccc4)cc(N(c4ccccc4)c4ccc(-c5ccccc5)cc4)c3)c3cccc4c3oc3ccccc34)cc12] | 78% |
| Com 21-38 | 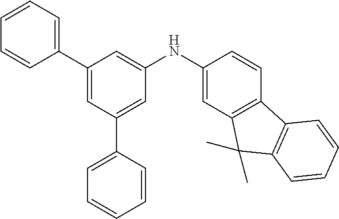 [SMILES: CC1(C)c2ccccc2-c2ccc(Nc3cc(-c4ccccc4)cc(-c4ccccc4)c3)cc21] | 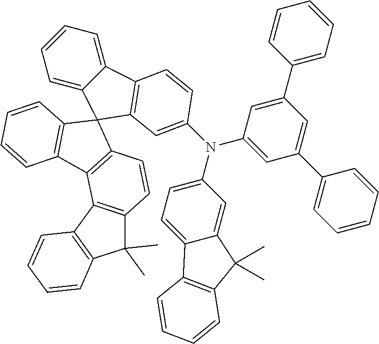 [SMILES: CC1(C)c2ccccc2-c2ccc(N(c3cc(-c4ccccc4)cc(-c4ccccc4)c3)c3ccc4c(c3)C3(c5ccccc5-4)c4ccccc4-c4c3ccc3c4-c4ccccc4C3(C)C)cc21] | 89% |
| Com 21-39 | 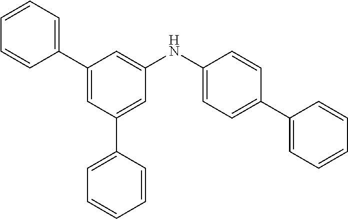 [SMILES: c1ccc(-c2ccc(Nc3cc(-c4ccccc4)cc(-c4ccccc4)c3)cc2)cc1] | 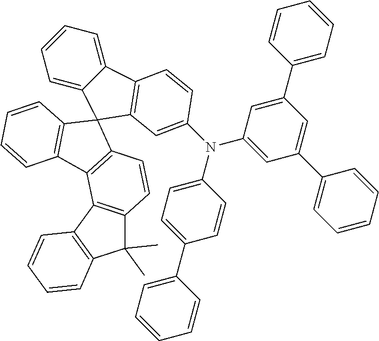 [SMILES: CC1(C)c2ccccc2-c2c1ccc1c2-c2ccccc2C12c1ccccc1-c1ccc(N(c3ccc(-c4ccccc4)cc3)c3cc(-c4ccccc4)cc(-c4ccccc4)c3)cc12] | 88% |
| Com 21-40 | 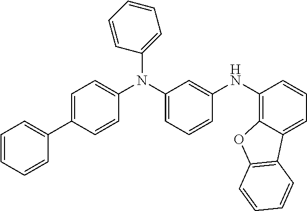 [SMILES: c1ccc(-c2ccc(N(c3ccccc3)c3cccc(Nc4cccc5c4oc4ccccc45)c3)cc2)cc1] | 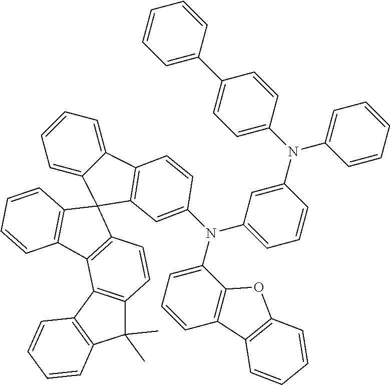 [SMILES: CC1(C)c2ccccc2-c2c1ccc1c2-c2ccccc2C12c1ccccc1-c1ccc(N(c3cccc(N(c4ccccc4)c4ccc(-c5ccccc5)cc4)c3)c3cccc4c3oc3ccccc34)cc12] | 69% |
| Com 21-41 | 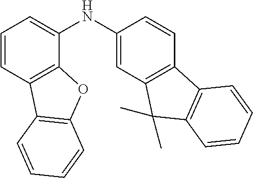 [SMILES: CC1(C)c2ccccc2-c2ccc(Nc3cccc4c3oc3ccccc34)cc21] | 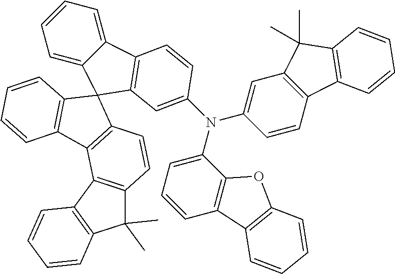 [SMILES: CC1(C)c2ccccc2-c2ccc(N(c3ccc4c(c3)C3(c5ccccc5-4)c4ccccc4-c4c3ccc3c4-c4ccccc4C3(C)C)c3cccc4c3oc3ccccc34)cc21] | 82% |
| Com 21-42 | 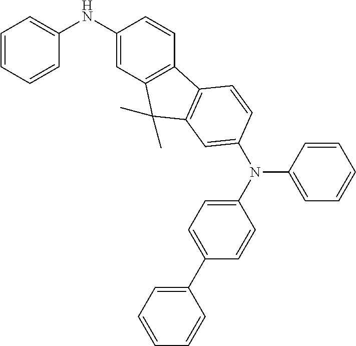 [SMILES: CC1(C)c2cc(Nc3ccccc3)ccc2-c2ccc(N(c3ccccc3)c3ccc(-c4ccccc4)cc3)cc21] | 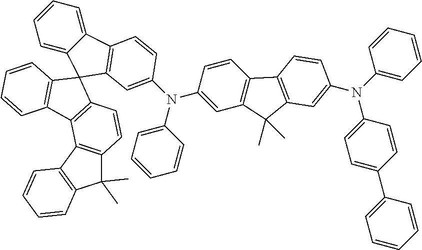 [SMILES: CC1(C)c2cc(N(c3ccccc3)c3ccc(-c4ccccc4)cc3)ccc2-c2ccc(N(c3ccccc3)c3ccc4c(c3)C3(c5ccccc5-4)c4ccccc4-c4c3ccc3c4-c4ccccc4C3(C)C)cc21] | 73% |
| Com 21-43 | 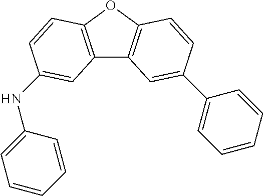 [SMILES: c1ccc(Nc2ccc3oc4ccc(-c5ccccc5)cc4c3c2)cc1] | 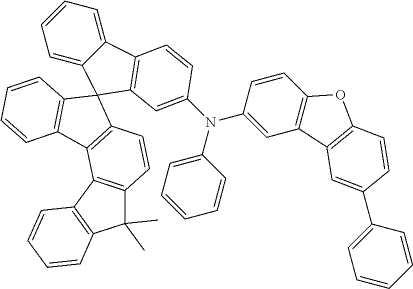 [SMILES: CC1(C)c2ccccc2-c2c1ccc1c2-c2ccccc2C12c1ccccc1-c1ccc(N(c3ccccc3)c3ccc4oc5ccc(-c6ccccc6)cc5c4c3)cc12] | 83% |
| Com 21-44 | 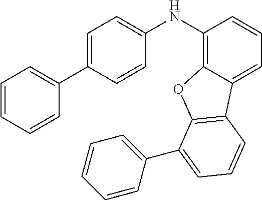 [SMILES: c1ccc(-c2ccc(Nc3cccc4c3oc3c(-c5ccccc5)cccc34)cc2)cc1] | 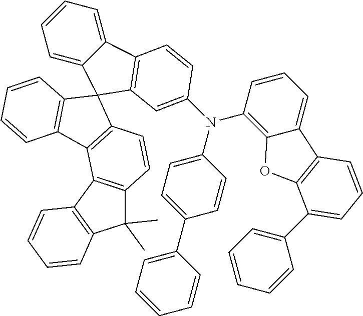 [SMILES: CC1(C)c2ccccc2-c2c1ccc1c2-c2ccccc2C12c1ccccc1-c1ccc(N(c3ccc(-c4ccccc4)cc3)c3cccc4c3oc3c(-c5ccccc5)cccc34)cc12] | 86% |
| Com 21-45 | 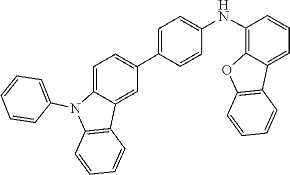 [SMILES: c1ccc(-n2c3ccccc3c3cc(-c4ccc(Nc5cccc6c5oc5ccccc56)cc4)ccc32)cc1] | 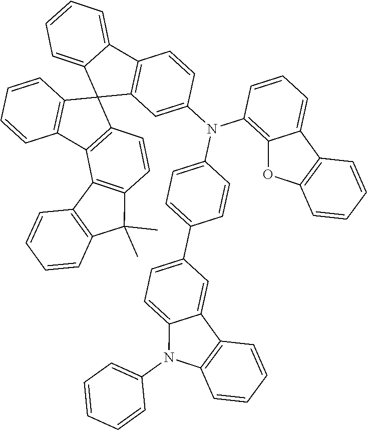 [SMILES: CC1(C)c2ccccc2-c2c1ccc1c2-c2ccccc2C12c1ccccc1-c1ccc(N(c3ccc(-c4ccc5c(c4)c4ccccc4n5-c4ccccc4)cc3)c3cccc4c3oc3ccccc34)cc12] | 72% |
| Com 21-46 | 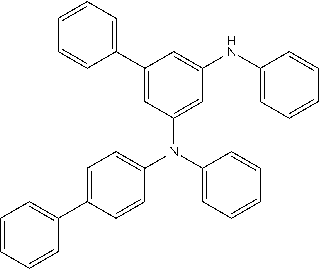 [SMILES: c1ccc(Nc2cc(-c3ccccc3)cc(N(c3ccccc3)c3ccc(-c4ccccc4)cc3)c2)cc1] | 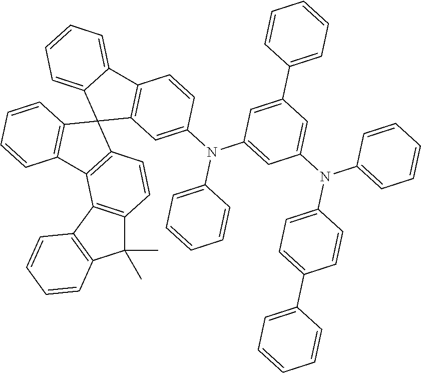 [SMILES: CC1(C)c2ccccc2-c2c1ccc1c2-c2ccccc2C12c1ccccc1-c1ccc(N(c3ccccc3)c3cc(-c4ccccc4)cc(N(c4ccccc4)c4ccc(-c5ccccc5)cc4)c3)cc12] | 88% |
| Com 21-47 | 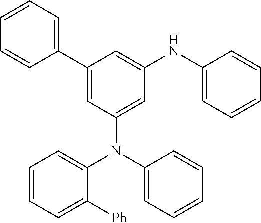 [SMILES: c1ccc(Nc2cc(-c3ccccc3)cc(N(c3ccccc3)c3ccccc3-c3ccccc3)c2)cc1] | 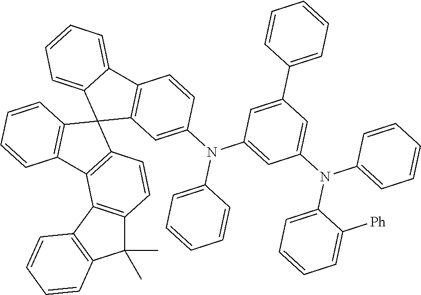 [SMILES: CC1(C)c2ccccc2-c2c1ccc1c2-c2ccccc2C12c1ccccc1-c1ccc(N(c3ccccc3)c3cc(-c4ccccc4)cc(N(c4ccccc4)c4ccccc4-c4ccccc4)c3)cc12] | 63% |
| Com 21-48 | 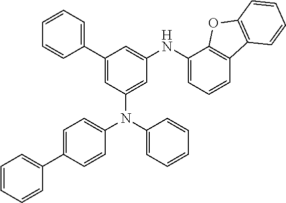 [SMILES: c1ccc(-c2ccc(N(c3ccccc3)c3cc(Nc4cccc5c4oc4ccccc45)cc(-c4ccccc4)c3)cc2)cc1] | 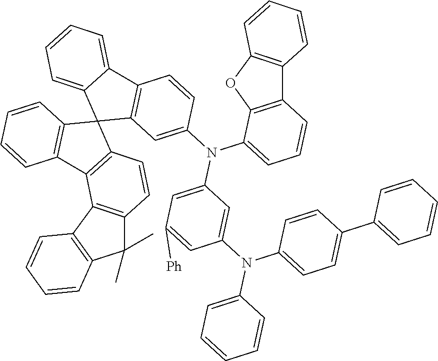 [SMILES: CC1(C)c2ccccc2-c2c1ccc1c2-c2ccccc2C12c1ccccc1-c1ccc(N(c3cc(-c4ccccc4)cc(N(c4ccccc4)c4ccc(-c5ccccc5)cc4)c3)c3cccc4c3oc3ccccc34)cc12] | 76% |
| Com 21-49 | 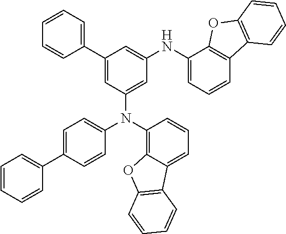 [SMILES: c1ccc(-c2ccc(N(c3cc(Nc4cccc5c4oc4ccccc45)cc(-c4ccccc4)c3)c3cccc4c3oc3ccccc34)cc2)cc1] | 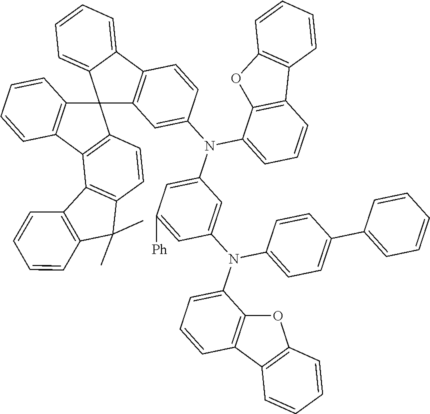 [SMILES: CC1(C)c2ccccc2-c2c1ccc1c2-c2ccccc2C12c1ccccc1-c1ccc(N(c3cc(-c4ccccc4)cc(N(c4ccc(-c5ccccc5)cc4)c4cccc5c4oc4ccccc45)c3)c3cccc4c3oc3ccccc34)cc12] | 66% |
| Com 21-50 | 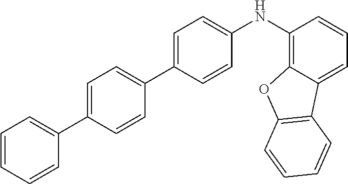 [SMILES: c1ccc(-c2ccc(-c3ccc(Nc4cccc5c4oc4ccccc45)cc3)cc2)cc1] | 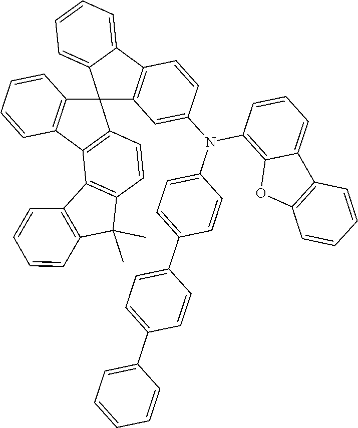 [SMILES: CC1(C)c2ccccc2-c2c1ccc1c2-c2ccccc2C12c1ccccc1-c1ccc(N(c3ccc(-c4ccc(-c5ccccc5)cc4)cc3)c3cccc4c3oc3ccccc34)cc12] | 79% |

### [Preparation Example 2] Preparation of Compound B-1

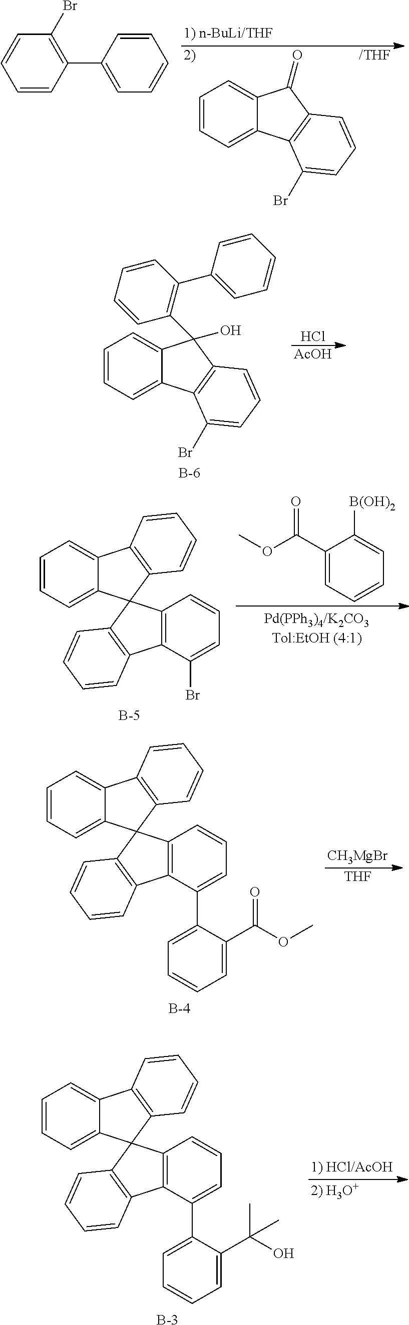

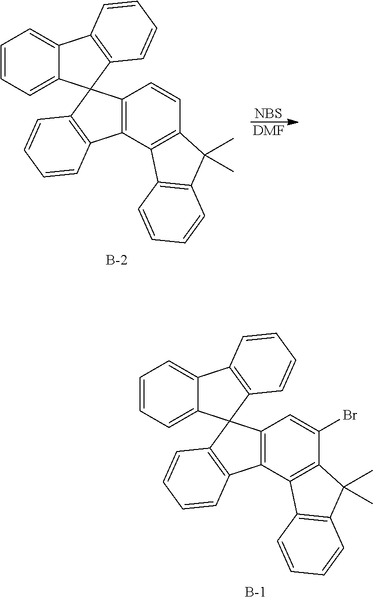

Preparation of Compound B-2

A compound B-2 was prepared in the same manner as that of Preparation Example 1, except that 2-bromo-1,1′-biphenyl (38.00 mmol) was used instead of 2-bromo-4′-methoxy-1,1′-biphenyl as a starting material.

Preparation of Compound B-1

5 g (11.56 mmol) of the compound B-2, 2.26 g (12.71 mmol) of N-bromosuccinimide, and 3 g (100 wt %) of SiO₂ were added to 70 mL of DMF, the mixed solution was wrapped with a silver foil, and then stirring was performed at room temperature. After the reaction was completed, extraction was performed using MC and H₂O, the solvent was removed, and then purification was performed by column chromatography with Hex and EA, thereby obtaining 1.8 g (30%) of a target compound B-1.

### [Example 2] Preparation of Compounds P3 and P4

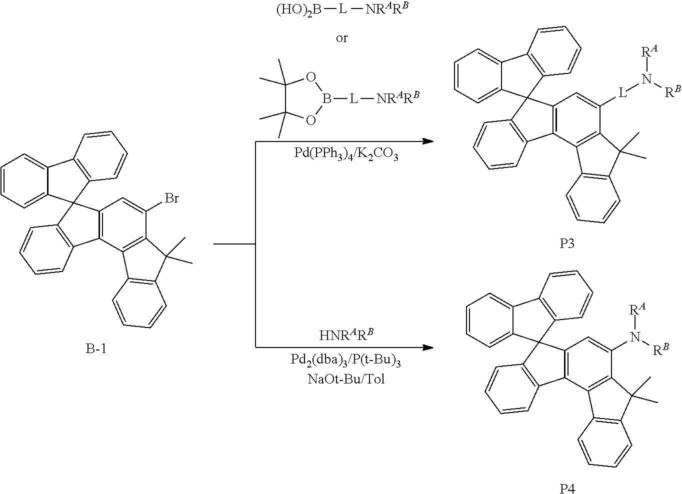

Preparation of Compound P3

A target compound P3 was obtained through a Suzuki coupling reaction using the compound B-1, a substituted boronic acid or a boronic ester compound, and a palladium catalyst.

Preparation of Compound P4

A target compound P4 was obtained through palladium-amination using the compound B-1, a secondary amine compound, and a palladium catalyst.

The structures of the substituted boronic acid, boronic ester, and secondary amine compound as the reactants and the structures and yields of the prepared compounds P3 and P4 are shown in Table 2.

TABLE 2

| Com- | | | |
|---|---|---|---|
| pound | | | |
| No. | Reactant | Product (P3, P4) | Yield |
| Com 22-1 | 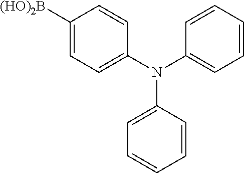 [SMILES: OB(O)c1ccc(N(c2ccccc2)c2ccccc2)cc1] | 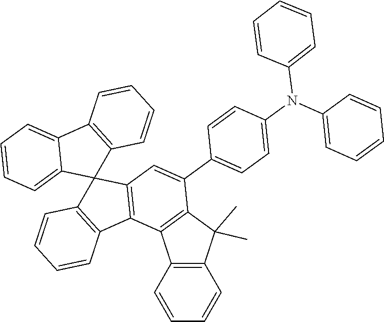 [SMILES: CC1(C)c2ccccc2-c2c3c(cc(-c4ccc(N(c5ccccc5)c5ccccc5)cc4)c21)C1(c2ccccc2-c2ccccc21)c1ccccc1-3] | 61% |
| Com 22-2 | 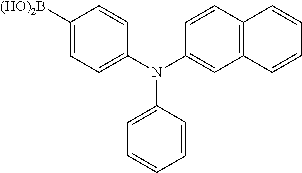 [SMILES: OB(O)c1ccc(N(c2ccccc2)c2ccc3ccccc3c2)cc1] | 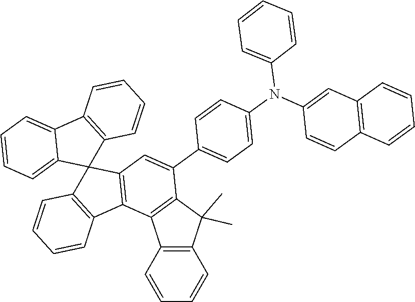 [SMILES: CC1(C)c2ccccc2-c2c3c(cc(-c4ccc(N(c5ccccc5)c5ccc6ccccc6c5)cc4)c21)C1(c2ccccc2-c2ccccc21)c1ccccc1-3] | 75% |
| Com 22-3 | 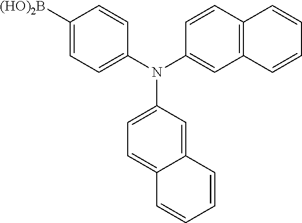 [SMILES: OB(O)c1ccc(N(c2ccc3ccccc3c2)c2ccc3ccccc3c2)cc1] | 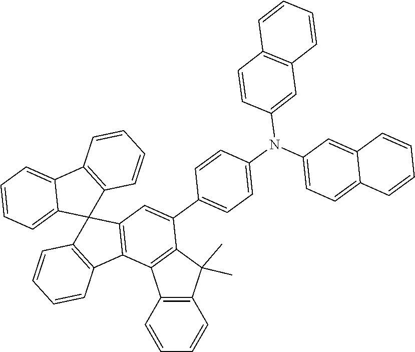 [SMILES: CC1(C)c2ccccc2-c2c3c(cc(-c4ccc(N(c5ccc6ccccc6c5)c5ccc6ccccc6c5)cc4)c21)C1(c2ccccc2-c2ccccc21)c1ccccc1-3] | 63% |
| Com 22-4 | 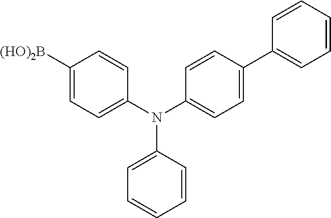 [SMILES: OB(O)c1ccc(N(c2ccccc2)c2ccc(-c3ccccc3)cc2)cc1] | 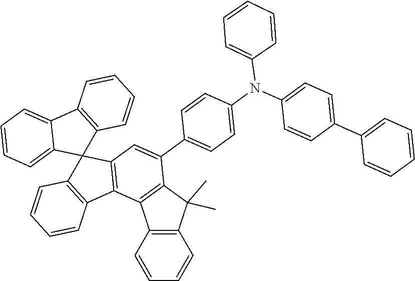 [SMILES: CC1(C)c2ccccc2-c2c3c(cc(-c4ccc(N(c5ccccc5)c5ccc(-c6ccccc6)cc5)cc4)c21)C1(c2ccccc2-c2ccccc21)c1ccccc1-3] | 69% |
| Com 22-5 | 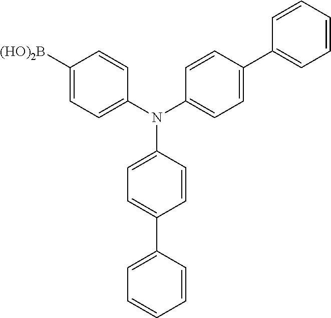 [SMILES: OB(O)c1ccc(N(c2ccc(-c3ccccc3)cc2)c2ccc(-c3ccccc3)cc2)cc1] | 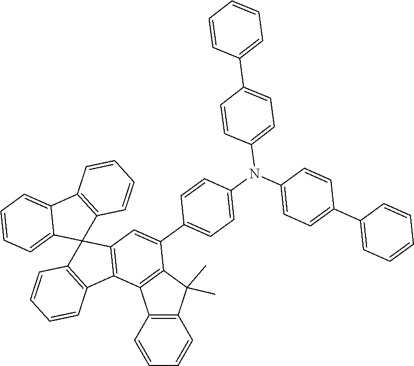 [SMILES: CC1(C)c2ccccc2-c2c3c(cc(-c4ccc(N(c5ccc(-c6ccccc6)cc5)c5ccc(-c6ccccc6)cc5)cc4)c21)C1(c2ccccc2-c2ccccc21)c1ccccc1-3] | 61% |
| Com 22-6 | 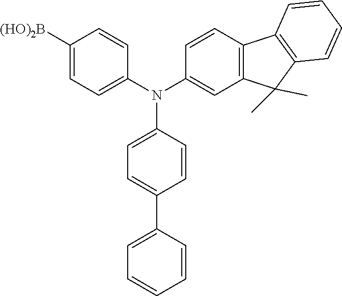 [SMILES: CC1(C)c2ccccc2-c2ccc(N(c3ccc(B(O)O)cc3)c3ccc(-c4ccccc4)cc3)cc21] | 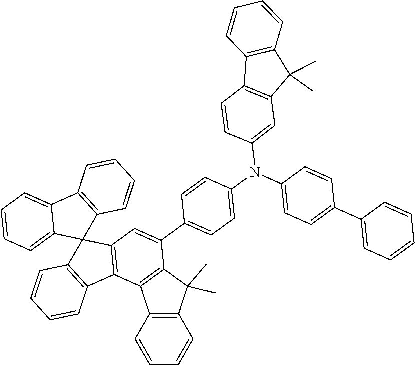 [SMILES: CC1(C)c2ccccc2-c2ccc(N(c3ccc(-c4ccccc4)cc3)c3ccc(-c4cc5c(c6c4C(C)(C)c4ccccc4-6)-c4ccccc4C54c5ccccc5-c5ccccc54)cc3)cc21] | 60% |
| Com 22-7 | 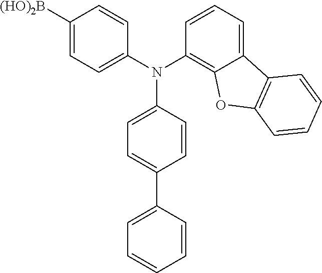 [SMILES: OB(O)c1ccc(N(c2ccc(-c3ccccc3)cc2)c2cccc3c2oc2ccccc23)cc1] | 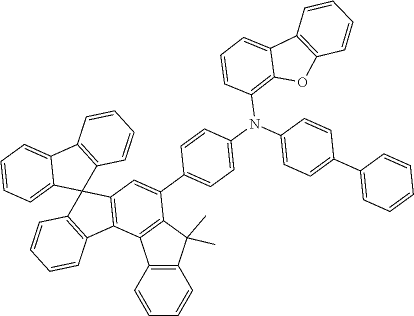 [SMILES: CC1(C)c2ccccc2-c2c3c(cc(-c4ccc(N(c5ccc(-c6ccccc6)cc5)c5cccc6c5oc5ccccc56)cc4)c21)C1(c2ccccc2-c2ccccc21)c1ccccc1-3] | 52% |
| Com 22-8 | 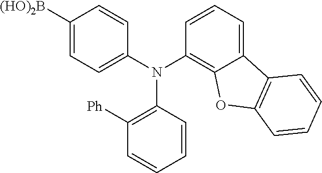 [SMILES: OB(O)c1ccc(N(c2ccccc2-c2ccccc2)c2cccc3c2oc2ccccc23)cc1] | 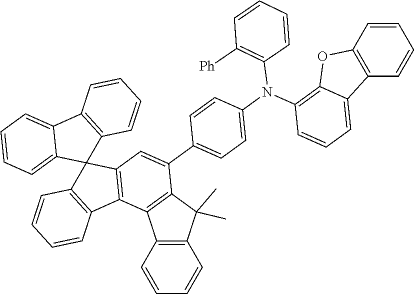 [SMILES: CC1(C)c2ccccc2-c2c3c(cc(-c4ccc(N(c5ccccc5-c5ccccc5)c5cccc6c5oc5ccccc56)cc4)c21)C1(c2ccccc2-c2ccccc21)c1ccccc1-3] | 60% |
| Com 22-9 | 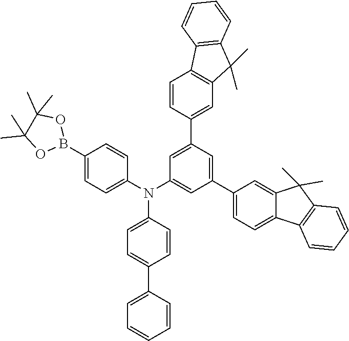 [SMILES: CC1(C)c2ccccc2-c2ccc(-c3cc(-c4ccc5c(c4)C(C)(C)c4ccccc4-5)cc(N(c4ccc(B5OC(C)(C)C(C)(C)O5)cc4)c4ccc(-c5ccccc5)cc4)c3)cc21] | 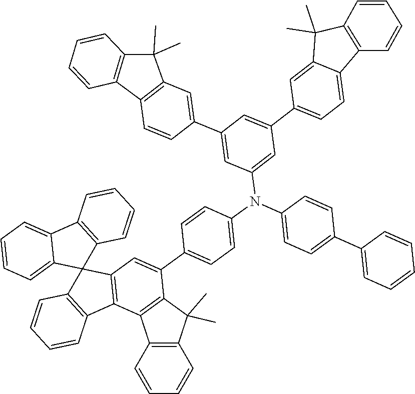 [SMILES: CC1(C)c2ccccc2-c2ccc(-c3cc(-c4ccc5c(c4)C(C)(C)c4ccccc4-5)cc(N(c4ccc(-c5ccccc5)cc4)c4ccc(-c5cc6c(c7c5C(C)(C)c5ccccc5-7)-c5ccccc5C65c6ccccc6-c6ccccc65)cc4)c3)cc21] | 58% |
| Com 22-10 | 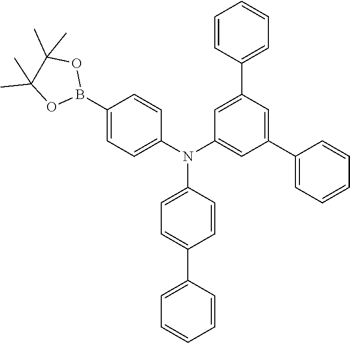 [SMILES: CC1(C)OB(c2ccc(N(c3ccc(-c4ccccc4)cc3)c3cc(-c4ccccc4)cc(-c4ccccc4)c3)cc2)OC1(C)C] | 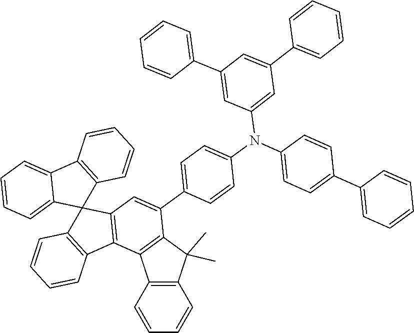 [SMILES: CC1(C)c2ccccc2-c2c3c(cc(-c4ccc(N(c5ccc(-c6ccccc6)cc5)c5cc(-c6ccccc6)cc(-c6ccccc6)c5)cc4)c21)C1(c2ccccc2-c2ccccc21)c1ccccc1-3] | 59% |
| Com 22-11 | 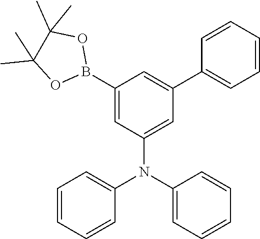 [SMILES: CC1(C)OB(c2cc(-c3ccccc3)cc(N(c3ccccc3)c3ccccc3)c2)OC1(C)C] | 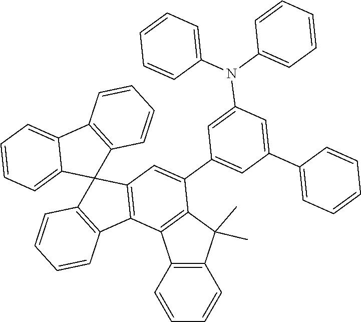 [SMILES: CC1(C)c2ccccc2-c2c3c(cc(-c4cc(-c5ccccc5)cc(N(c5ccccc5)c5ccccc5)c4)c21)C1(c2ccccc2-c2ccccc21)c1ccccc1-3] | 69% |
| Com 22-12 | 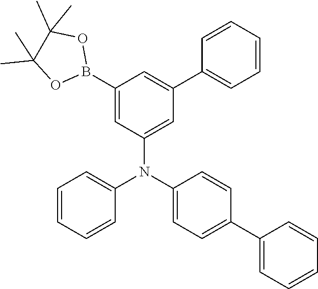 [SMILES: CC1(C)OB(c2cc(-c3ccccc3)cc(N(c3ccccc3)c3ccc(-c4ccccc4)cc3)c2)OC1(C)C] | 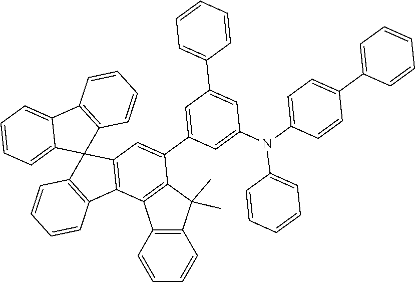 [SMILES: CC1(C)c2ccccc2-c2c3c(cc(-c4cc(-c5ccccc5)cc(N(c5ccccc5)c5ccc(-c6ccccc6)cc5)c4)c21)C1(c2ccccc2-c2ccccc21)c1ccccc1-3] | 70% |
| Com 22-13 | 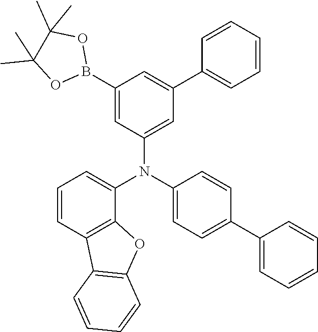 [SMILES: CC1(C)OB(c2cc(-c3ccccc3)cc(N(c3ccc(-c4ccccc4)cc3)c3cccc4c3oc3ccccc34)c2)OC1(C)C] | 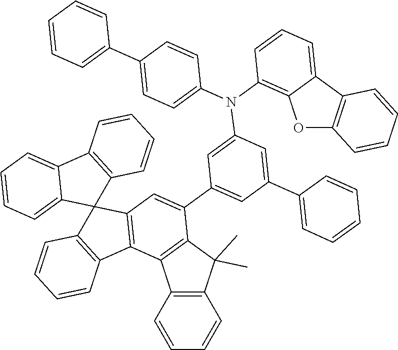 [SMILES: CC1(C)c2ccccc2-c2c3c(cc(-c4cc(-c5ccccc5)cc(N(c5ccc(-c6ccccc6)cc5)c5cccc6c5oc5ccccc56)c4)c21)C1(c2ccccc2-c2ccccc21)c1ccccc1-3] | 71% |
| Com 22-14 | 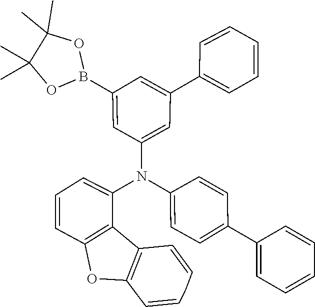 [SMILES: CC1(C)OB(c2cc(-c3ccccc3)cc(N(c3ccc(-c4ccccc4)cc3)c3cccc4oc5c(c34)/C=C\C/C=C\C5)c2)OC1(C)C] | 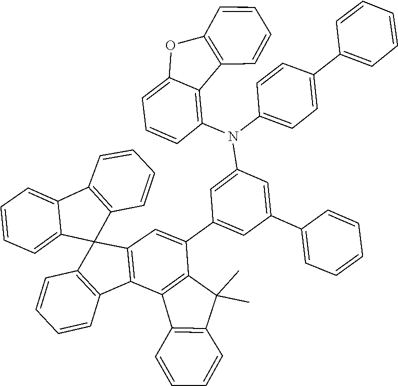 [SMILES: CC1(C)c2ccccc2-c2c3c(cc(-c4cc(-c5ccccc5)cc(N(c5ccc(-c6ccccc6)cc5)c5cccc6oc7ccccc7c56)c4)c21)C1(c2ccccc2-c2ccccc21)c1ccccc1-3] | 69% |
| Com 22-15 | 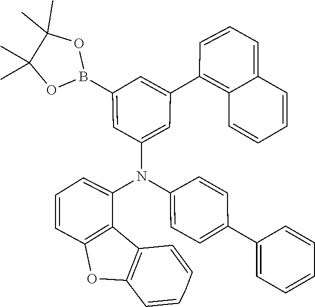 [SMILES: CC1(C)OB(c2cc(-c3cccc4ccccc34)cc(N(c3ccc(-c4ccccc4)cc3)c3cccc4oc5c(c34)C=CCC=C5)c2)OC1(C)C] | 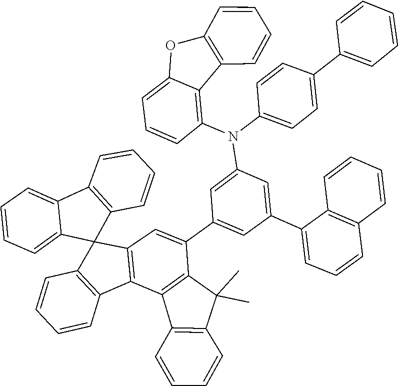 [SMILES: CC1(C)c2ccccc2-c2c3c(cc(-c4cc(-c5cccc6ccccc56)cc(N(c5ccc(-c6ccccc6)cc5)c5cccc6oc7ccccc7c56)c4)c21)C1(c2ccccc2-c2ccccc21)c1ccccc1-3] | 68% |
| Com 22-16 | 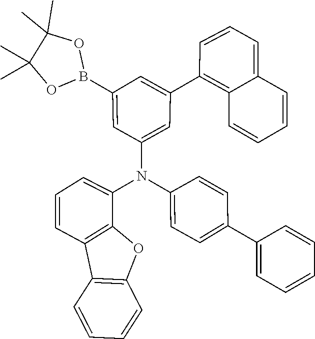 [SMILES: CC1(C)OB(c2cc(-c3cccc4ccccc34)cc(N(c3ccc(-c4ccccc4)cc3)c3cccc4c3oc3ccccc34)c2)OC1(C)C] | 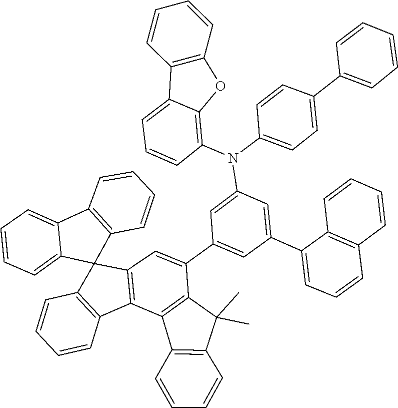 [SMILES: CC1(C)c2ccccc2-c2c3c(cc(-c4cc(-c5cccc6ccccc56)cc(N(c5ccc(-c6ccccc6)cc5)c5cccc6c5oc5ccccc56)c4)c21)C1(c2ccccc2-c2ccccc21)c1ccccc1-3] | 50% |
| Com 22-17 | 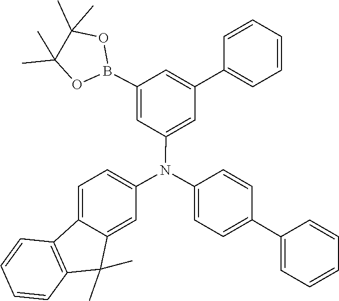 [SMILES: CC1(C)c2ccccc2-c2ccc(N(c3ccc(-c4ccccc4)cc3)c3cc(B4OC(C)(C)C(C)(C)O4)cc(-c4ccccc4)c3)cc21] | 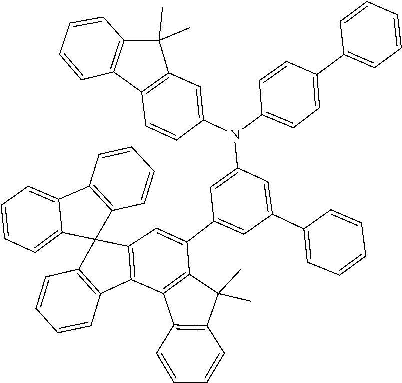 [SMILES: CC1(C)c2ccccc2-c2ccc(N(c3ccc(-c4ccccc4)cc3)c3cc(-c4ccccc4)cc(-c4cc5c(c6c4C(C)(C)c4ccccc4-6)-c4ccccc4C54c5ccccc5-c5ccccc54)c3)cc21] | 58% |
| Com 22-18 | 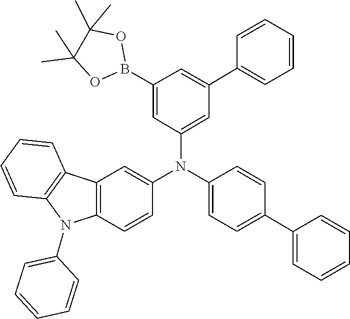 [SMILES: CC1(C)OB(c2cc(-c3ccccc3)cc(N(c3ccc(-c4ccccc4)cc3)c3ccc4c(c3)c3ccccc3n4-c3ccccc3)c2)OC1(C)C] | 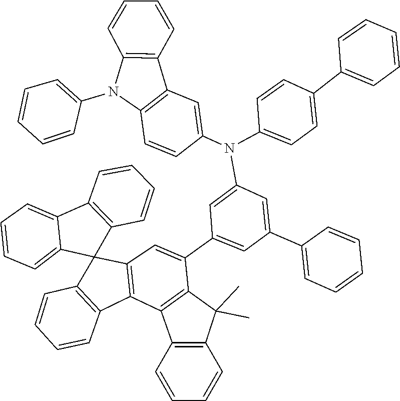 [SMILES: CC1(C)c2ccccc2-c2c3c(cc(-c4cc(-c5ccccc5)cc(N(c5ccc(-c6ccccc6)cc5)c5ccc6c(c5)c5ccccc5n6-c5ccccc5)c4)c21)C1(c2ccccc2-c2ccccc21)c1ccccc1-3] | 64% |
| Com 22-19 | 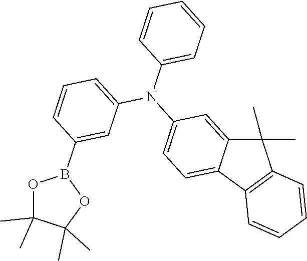 [SMILES: CC1(C)c2ccccc2-c2ccc(N(c3ccccc3)c3cccc(B4OC(C)(C)C(C)(C)O4)c3)cc21] | 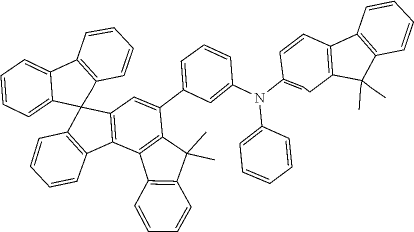 [SMILES: CCC1(CC)C2=C(/C=C\C(N(c3ccccc3)c3cccc(-c4cc5c(c6c4C(C)(C)c4ccccc4-6)-c4ccccc4C54Cc5ccccc5-c5ccccc54)c3)=C\CC2)c2ccccc21] | 75% |
| Com 22-20 | 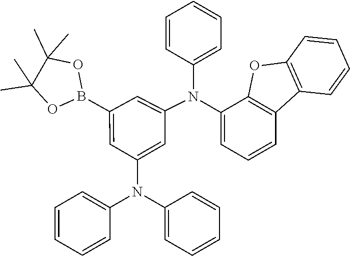 [SMILES: CC1(C)OB(c2cc(N(c3ccccc3)c3ccccc3)cc(N(c3ccccc3)c3cccc4c3oc3ccccc34)c2)OC1(C)C] | 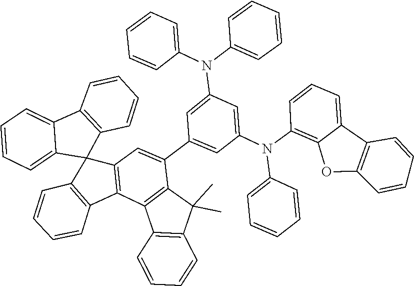 [SMILES: CC1(C)c2ccccc2-c2c3c(cc(-c4cc(N(c5ccccc5)c5ccccc5)cc(N(c5ccccc5)c5cccc6c5oc5ccccc56)c4)c21)C1(Cc2ccccc2-c2ccccc21)c1ccccc1-3] | 72% |
| Com 22-21 | 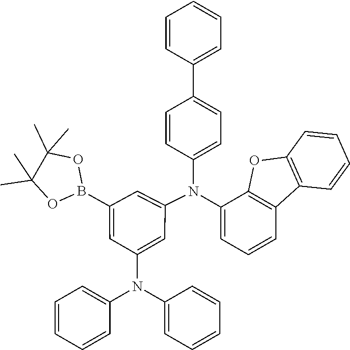 [SMILES: CC1(C)OB(c2cc(N(c3ccccc3)c3ccccc3)cc(N(c3ccc(-c4ccccc4)cc3)c3cccc4c3oc3ccccc34)c2)OC1(C)C] | 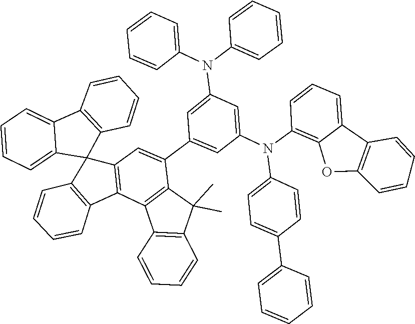 [SMILES: CC1(C)c2ccccc2-c2c3c(cc(-c4cc(N(c5ccccc5)c5ccccc5)cc(N(c5ccc(-c6ccccc6)cc5)c5cccc6c5oc5ccccc56)c4)c21)C1(Cc2ccccc2-c2ccccc21)c1ccccc1-3] | 67% |
| Com 22-22 | 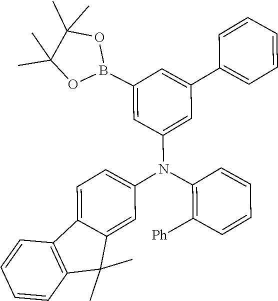 [SMILES: CC1(C)c2ccccc2-c2ccc(N(c3cc(B4OC(C)(C)C(C)(C)O4)cc(-c4ccccc4)c3)c3ccccc3-c3ccccc3)cc21] | 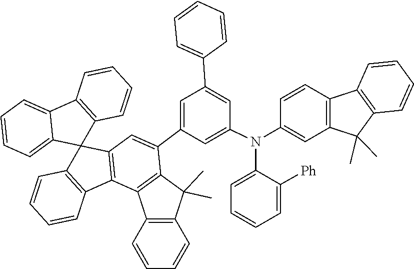 [SMILES: CC1(C)c2ccccc2-c2ccc(N(c3cc(-c4ccccc4)cc(-c4cc5c(c6c4C(C)(C)c4ccccc4-6)-c4ccccc4C54c5ccccc5-c5ccccc54)c3)c3ccccc3-c3ccccc3)cc21] | 61% |
| Com 22-23 | 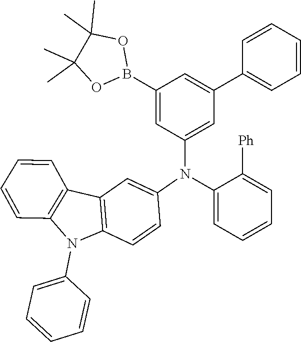 [SMILES: CC1(C)OB(c2cc(-c3ccccc3)cc(N(c3ccc4c(c3)c3ccccc3n4-c3ccccc3)c3ccccc3-c3ccccc3)c2)OC1(C)C] | 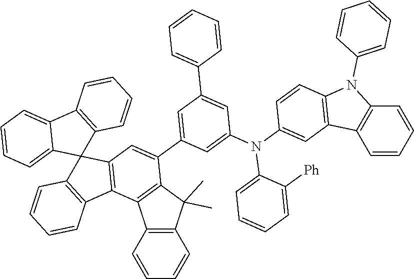 [SMILES: CC1(C)c2ccccc2-c2c3c(cc(-c4cc(-c5ccccc5)cc(N(c5ccc6c(c5)c5ccccc5n6-c5ccccc5)c5ccccc5-c5ccccc5)c4)c21)C1(c2ccccc2-c2ccccc21)c1ccccc1-3] | 72% |
| Com 22-24 | 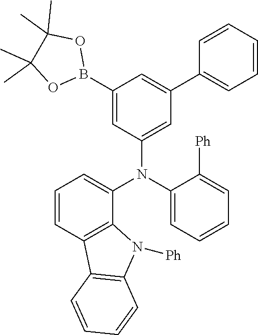 [SMILES: CC1(C)OB(c2cc(-c3ccccc3)cc(N(c3ccccc3-c3ccccc3)c3cccc4c5ccccc5n(-c5ccccc5)c34)c2)OC1(C)C] | 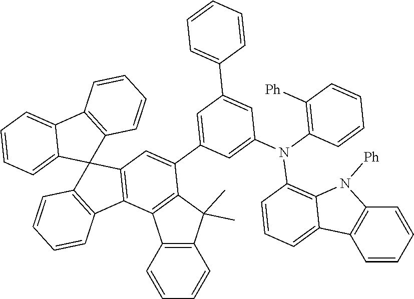 [SMILES: CC1(C)c2ccccc2-c2c3c(cc(-c4cc(-c5ccccc5)cc(N(c5ccccc5-c5ccccc5)c5cccc6c7ccccc7n(-c7ccccc7)c56)c4)c21)C1(c2ccccc2-c2ccccc21)c1ccccc1-3] | 54% |
| Com 22-25 | 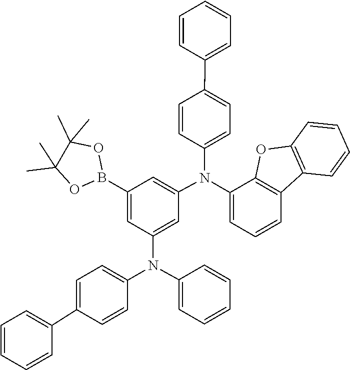 [SMILES: CC1(C)OB(c2cc(N(c3ccccc3)c3ccc(-c4ccccc4)cc3)cc(N(c3ccc(-c4ccccc4)cc3)c3cccc4c3oc3ccccc34)c2)OC1(C)C] | 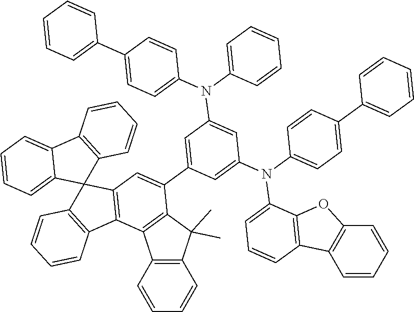 [SMILES: CC1(C)c2ccccc2-c2c3c(cc(-c4cc(N(c5ccccc5)c5ccc(-c6ccccc6)cc5)cc(N(c5ccc(-c6ccccc6)cc5)c5cccc6c5oc5ccccc56)c4)c21)C1(c2ccccc2-c2ccccc21)c1ccccc1-3] | 51% |
| Com 22-26 | 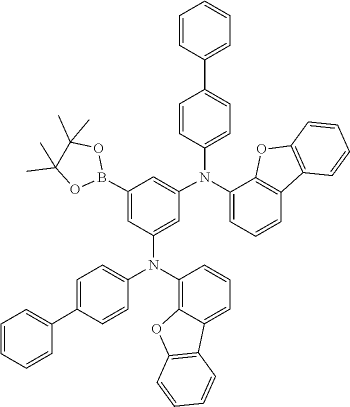 [SMILES: CC1(C)OB(c2cc(N(c3ccc(-c4ccccc4)cc3)c3cccc4c3oc3ccccc34)cc(N(c3ccc(-c4ccccc4)cc3)c3cccc4c3oc3ccccc34)c2)OC1(C)C] | 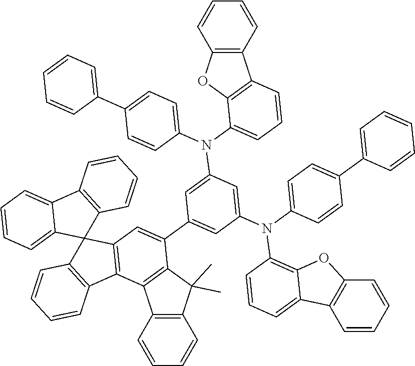 [SMILES: CC1(C)c2ccccc2-c2c3c(cc(-c4cc(N(c5ccc(-c6ccccc6)cc5)c5cccc6c5oc5ccccc56)cc(N(c5ccc(-c6ccccc6)cc5)c5cccc6c5oc5ccccc56)c4)c21)C1(c2ccccc2-c2ccccc21)c1ccccc1-3] | 47% |
| Com 22-27 | 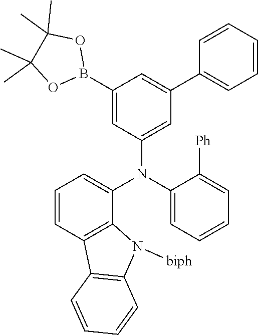 | 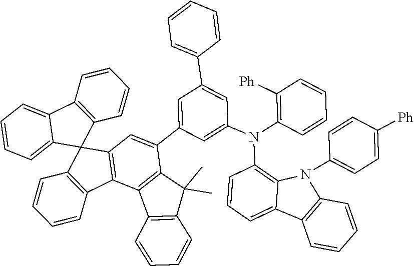 [SMILES: CC1(C)c2ccccc2-c2c3c(cc(-c4cc(-c5ccccc5)cc(N(c5ccccc5-c5ccccc5)c5cccc6c7ccccc7n(-c7ccc(-c8ccccc8)cc7)c56)c4)c21)C1(c2ccccc2-c2ccccc21)c1ccccc1-3] | 52% |
| Com 22-28 | 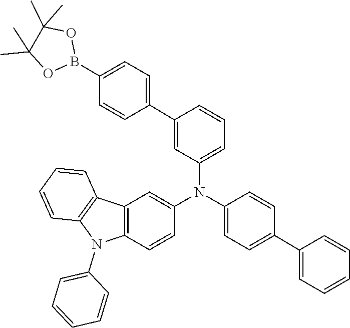 [SMILES: CC1(C)OB(c2ccc(-c3cccc(N(c4ccc(-c5ccccc5)cc4)c4ccc5c(c4)c4ccccc4n5-c4ccccc4)c3)cc2)OC1(C)C] | 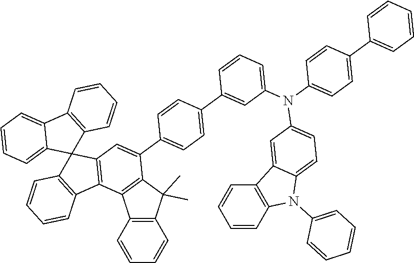 [SMILES: CC1(C)c2ccccc2-c2c3c(cc(-c4ccc(-c5cccc(N(c6ccc(-c7ccccc7)cc6)c6ccc7c(c6)c6ccccc6n7-c6ccccc6)c5)cc4)c21)C1(c2ccccc2-c2ccccc21)c1ccccc1-3] | 58% |
| Com 22-29 | 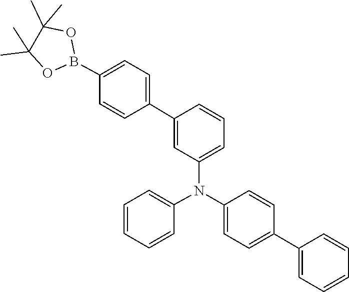 [SMILES: CC1(C)OB(c2ccc(-c3cccc(N(c4ccccc4)c4ccc(-c5ccccc5)cc4)c3)cc2)OC1(C)C] | 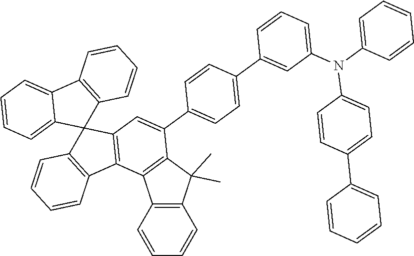 [SMILES: CC1(C)c2ccccc2-c2c3c(cc(-c4ccc(-c5cccc(N(c6ccccc6)c6ccc(-c7ccccc7)cc6)c5)cc4)c21)C1(c2ccccc2-c2ccccc21)c1ccccc1-3] | 87% |
| Com 22-30 | 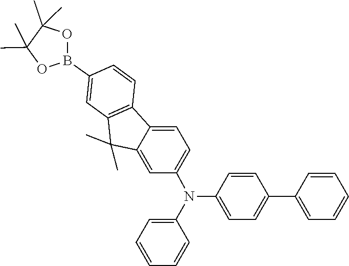 [SMILES: CC1(C)c2cc(B3OC(C)(C)C(C)(C)O3)ccc2-c2ccc(N(c3ccccc3)c3ccc(-c4ccccc4)cc3)cc21] | 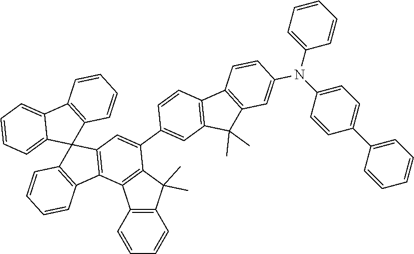 [SMILES: CC1(C)c2cc(-c3cc4c(c5c3C(C)(C)c3ccccc3-5)-c3ccccc3C43c4ccccc4-c4ccccc43)ccc2-c2ccc(N(c3ccccc3)c3ccc(-c4ccccc4)cc3)cc21] | 69% |
| Com 22-31 | 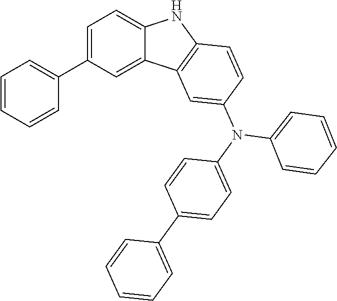 [SMILES: c1ccc(-c2ccc(N(c3ccccc3)c3ccc4[nH]c5ccc(-c6ccccc6)cc5c4c3)cc2)cc1] | 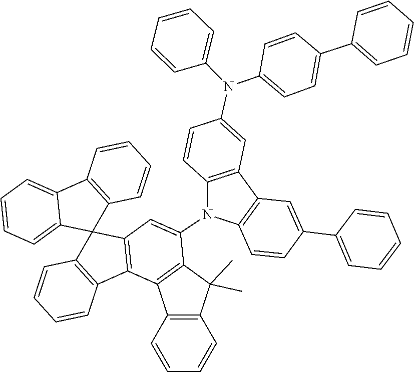 [SMILES: CC1(C)c2ccccc2-c2c3c(cc(-n4c5ccc(-c6ccccc6)cc5c5cc(N(c6ccccc6)c6ccc(-c7ccccc7)cc6)ccc54)c21)C1(c2ccccc2-c2ccccc21)c1ccccc1-3] | 65% |
| Com 22-32 | 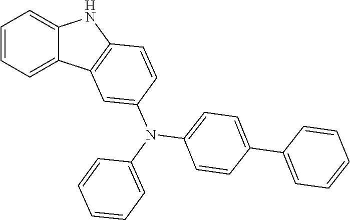 [SMILES: c1ccc(-c2ccc(N(c3ccccc3)c3ccc4[nH]c5ccccc5c4c3)cc2)cc1] | 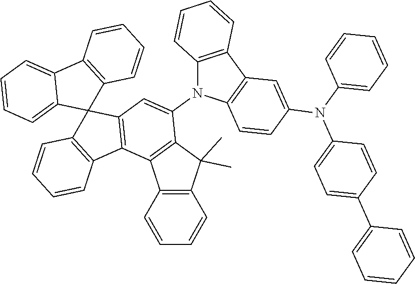 [SMILES: CC1(C)c2ccccc2-c2c3c(cc(-n4c5ccccc5c5cc(N(c6ccccc6)c6ccc(-c7ccccc7)cc6)ccc54)c21)C1(c2ccccc2-c2ccccc21)c1ccccc1-3] | 61% |
| Com 22-33 | 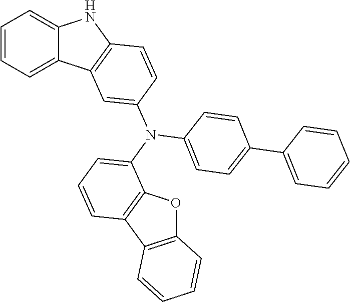 [SMILES: c1ccc(-c2ccc(N(c3ccc4[nH]c5ccccc5c4c3)c3cccc4c3oc3ccccc34)cc2)cc1] | 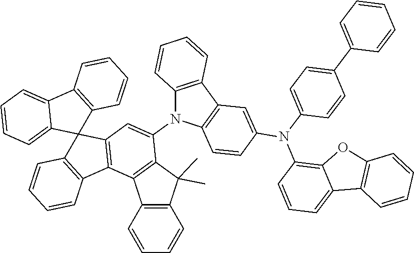 [SMILES: CC1(C)c2ccccc2-c2c3c(cc(-n4c5ccccc5c5cc(N(c6ccc(-c7ccccc7)cc6)c6cccc7c6oc6ccccc67)ccc54)c21)C1(c2ccccc2-c2ccccc21)c1ccccc1-3] | 58% |
| Com 22-34 | 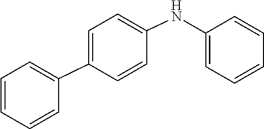 [SMILES: c1ccc(Nc2ccc(-c3ccccc3)cc2)cc1] | 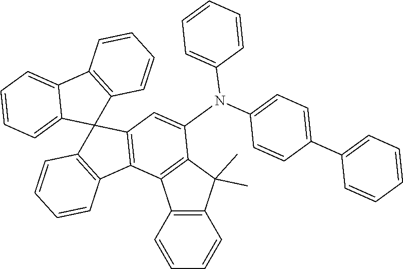 [SMILES: CC1(C)c2ccccc2-c2c3c(cc(N(c4ccccc4)c4ccc(-c5ccccc5)cc4)c21)C1(c2ccccc2-c2ccccc21)c1ccccc1-3] | 79% |
| Com 22-35 | 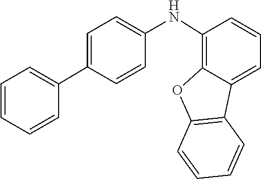 [SMILES: c1ccc(-c2ccc(Nc3cccc4c3oc3ccccc34)cc2)cc1] | 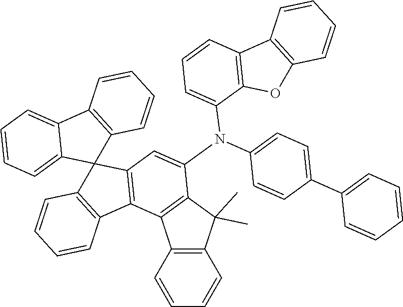 [SMILES: CC1(C)c2ccccc2-c2c3c(cc(N(c4ccc(-c5ccccc5)cc4)c4cccc5c4oc4ccccc45)c21)C1(c2ccccc2-c2ccccc21)c1ccccc1-3] | 85% |
| Com 22-36 | 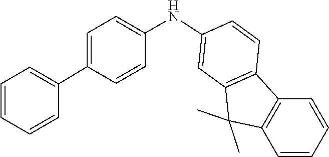 [SMILES: CC1(C)c2ccccc2-c2ccc(Nc3ccc(-c4ccccc4)cc3)cc21] | 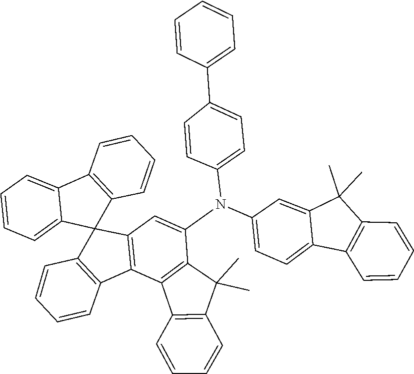 [SMILES: CC1(C)c2ccccc2-c2ccc(N(c3ccc(-c4ccccc4)cc3)c3cc4c(c5c3C(C)(C)c3ccccc3-5)-c3ccccc3C43c4ccccc4-c4ccccc43)cc21] | 91% |
| Com 22-37 | 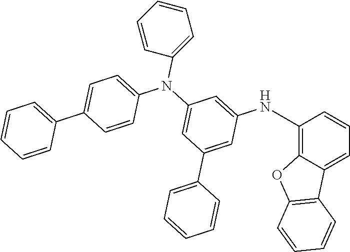 [SMILES: c1ccc(-c2ccc(N(c3ccccc3)c3cc(Nc4cccc5c4oc4ccccc45)cc(-c4ccccc4)c3)cc2)cc1] | 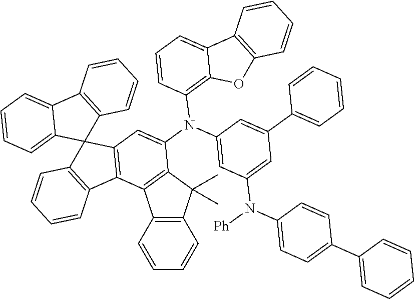 [SMILES: CC1(C)c2ccccc2-c2c3c(cc(N(c4cc(-c5ccccc5)cc(N(c5ccccc5)c5ccc(-c6ccccc6)cc5)c4)c4cccc5c4oc4ccccc45)c21)C1(c2ccccc2-c2ccccc21)c1ccccc1-3] | 78% |
| Com 22-38 | 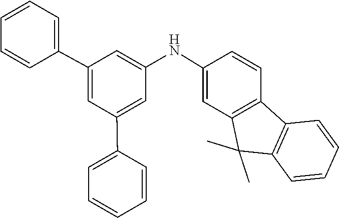 [SMILES: CC1(C)c2ccccc2-c2ccc(Nc3cc(-c4ccccc4)cc(-c4ccccc4)c3)cc21] | 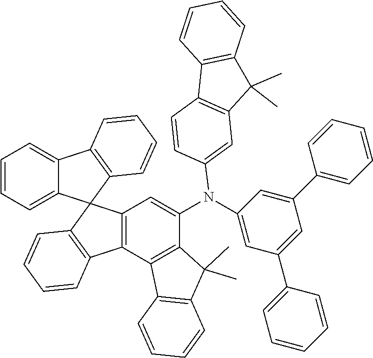 [SMILES: CC1(C)c2ccccc2-c2ccc(N(c3cc(-c4ccccc4)cc(-c4ccccc4)c3)c3cc4c(c5c3C(C)(C)c3ccccc3-5)-c3ccccc3C43c4ccccc4-c4ccccc43)cc21] | 63% |
| Com 22-39 | 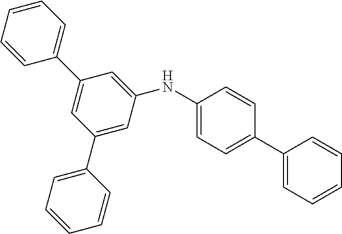 [SMILES: c1ccc(-c2ccc(Nc3cc(-c4ccccc4)cc(-c4ccccc4)c3)cc2)cc1] | 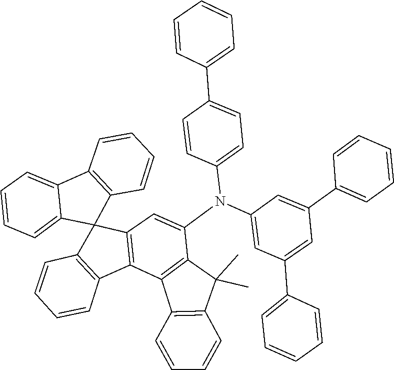 [SMILES: CC1(C)c2ccccc2-c2c3c(cc(N(c4ccc(-c5ccccc5)cc4)c4cc(-c5ccccc5)cc(-c5ccccc5)c4)c21)C1(c2ccccc2-c2ccccc21)c1ccccc1-3] | 81% |
| Com 22-40 | 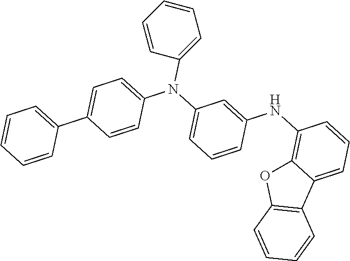 [SMILES: c1ccc(-c2ccc(N(c3ccccc3)c3cccc(Nc4cccc5c4oc4ccccc45)c3)cc2)cc1] | 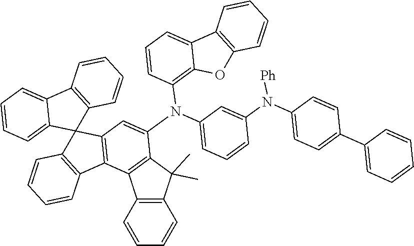 [SMILES: CC1(C)c2ccccc2-c2c3c(cc(N(c4cccc(N(c5ccccc5)c5ccc(-c6ccccc6)cc5)c4)c4cccc5c4oc4ccccc45)c21)C1(c2ccccc2-c2ccccc21)c1ccccc1-3] | 80% |
| Com 22-41 | 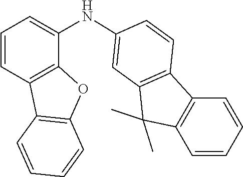 [SMILES: CC1(C)c2ccccc2-c2ccc(Nc3cccc4c3oc3ccccc34)cc21] | 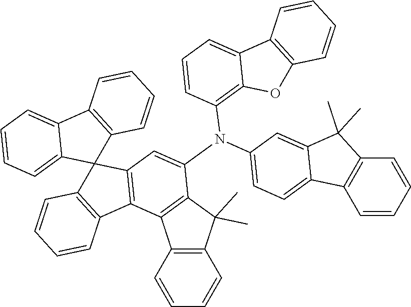 [SMILES: CC1(C)c2ccccc2-c2ccc(N(c3cc4c(c5c3C(C)(C)c3ccccc3-5)-c3ccccc3C43c4ccccc4-c4ccccc43)c3cccc4c3oc3ccccc34)cc21] | 81% |
| Com 22-42 | 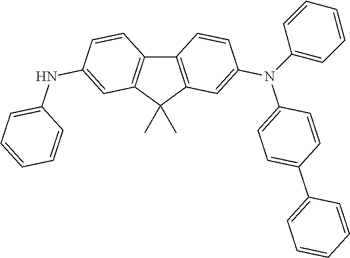 [SMILES: CC1(C)c2cc(Nc3ccccc3)ccc2-c2ccc(N(c3ccccc3)c3ccc(-c4ccccc4)cc3)cc21] | 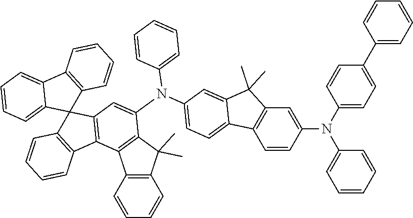 [SMILES: CC1(C)c2cc(N(c3ccccc3)c3ccc(-c4ccccc4)cc3)ccc2-c2ccc(N(c3ccccc3)c3cc4c(c5c3C(C)(C)c3ccccc3-5)-c3ccccc3C43c4ccccc4-c4ccccc43)cc21] | 74% |
| Com 22-43 | 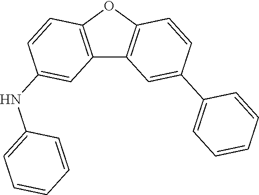 [SMILES: c1ccc(Nc2ccc3oc4ccc(-c5ccccc5)cc4c3c2)cc1] | 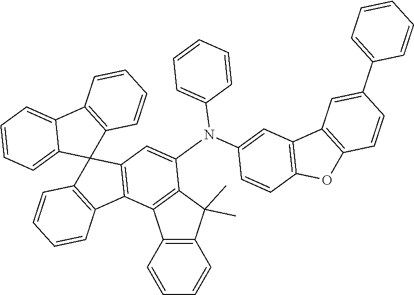 [SMILES: CC1(C)c2ccccc2-c2c3c(cc(N(c4ccccc4)c4ccc5oc6ccc(-c7ccccc7)cc6c5c4)c21)C1(c2ccccc2-c2ccccc21)c1ccccc1-3] | 65% |
| Com 22-44 | 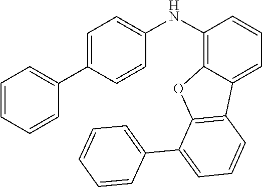 [SMILES: c1ccc(-c2ccc(Nc3cccc4c3oc3c(-c5ccccc5)cccc34)cc2)cc1] | 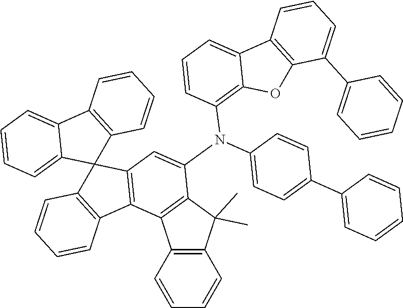 [SMILES: CC1(C)c2ccccc2-c2c3c(cc(N(c4ccc(-c5ccccc5)cc4)c4cccc5c4oc4c(-c6ccccc6)cccc45)c21)C1(c2ccccc2-c2ccccc21)c1ccccc1-3] | 74% |
| Com 22-45 | 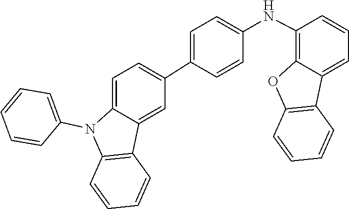 [SMILES: c1ccc(-n2c3ccccc3c3cc(-c4ccc(Nc5cccc6c5oc5ccccc56)cc4)ccc32)cc1] | 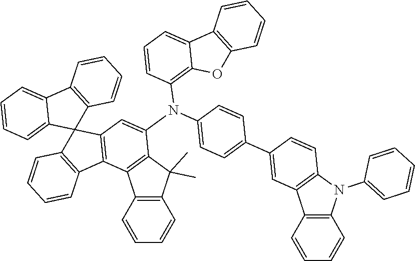 [SMILES: CC1(C)c2ccccc2-c2c3c(cc(N(c4ccc(-c5ccc6c(c5)c5ccccc5n6-c5ccccc5)cc4)c4cccc5c4oc4ccccc45)c21)C1(c2ccccc2-c2ccccc21)c1ccccc1-3] | 71% |
| Com 22-46 | 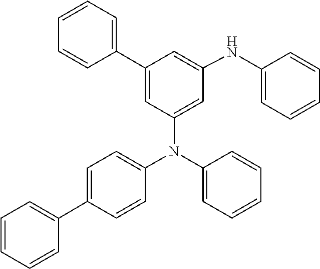 [SMILES: c1ccc(Nc2cc(-c3ccccc3)cc(N(c3ccccc3)c3ccc(-c4ccccc4)cc3)c2)cc1] | 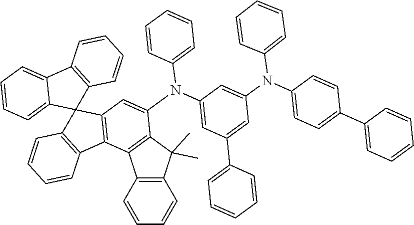 [SMILES: CC1(C)c2ccccc2-c2c3c(cc(N(c4ccccc4)c4cc(-c5ccccc5)cc(N(c5ccccc5)c5ccc(-c6ccccc6)cc5)c4)c21)C1(c2ccccc2-c2ccccc21)c1ccccc1-3] | 66% |
| Com 22-47 | 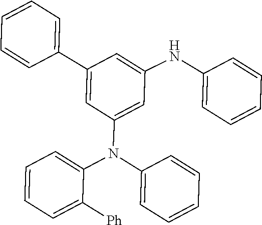 [SMILES: c1ccc(Nc2cc(-c3ccccc3)cc(N(c3ccccc3)c3ccccc3-c3ccccc3)c2)cc1] | 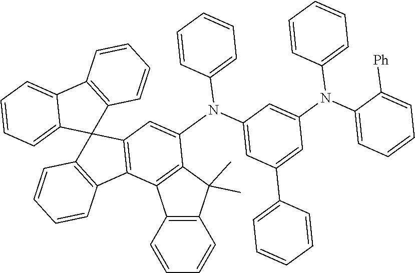 [SMILES: CC1(C)c2ccccc2-c2c3c(cc(N(c4ccccc4)c4cc(-c5ccccc5)cc(N(c5ccccc5)c5ccccc5-c5ccccc5)c4)c21)C1(c2ccccc2-c2ccccc21)c1ccccc1-3] | 54% |
| Com 22-48 | 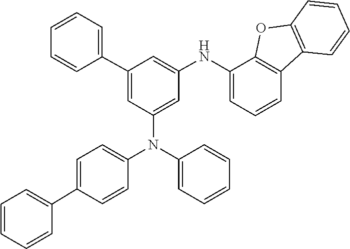 [SMILES: c1ccc(-c2ccc(N(c3ccccc3)c3cc(Nc4cccc5c4oc4ccccc45)cc(-c4ccccc4)c3)cc2)cc1] | 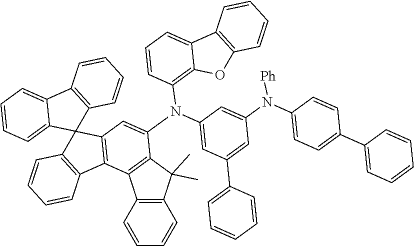 [SMILES: CC1(C)c2ccccc2-c2c3c(cc(N(c4cc(-c5ccccc5)cc(N(c5ccccc5)c5ccc(-c6ccccc6)cc5)c4)c4cccc5c4oc4ccccc45)c21)C1(c2ccccc2-c2ccccc21)c1ccccc1-3] | 54% |
| Com 22-49 | 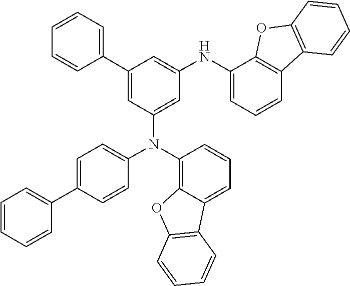 [SMILES: c1ccc(-c2ccc(N(c3cc(Nc4cccc5c4oc4ccccc45)cc(-c4ccccc4)c3)c3cccc4c3oc3ccccc34)cc2)cc1] | 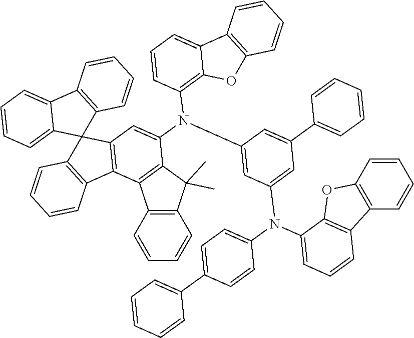 [SMILES: CC1(C)c2ccccc2-c2c3c(cc(N(c4cc(-c5ccccc5)cc(N(c5ccc(-c6ccccc6)cc5)c5cccc6c5oc5ccccc56)c4)c4cccc5c4oc4ccccc45)c21)C1(c2ccccc2-c2ccccc21)c1ccccc1-3] | 48% |
| Com 22-50 | 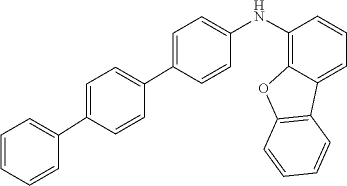 [SMILES: c1ccc(-c2ccc(-c3ccc(Nc4cccc5c4oc4ccccc45)cc3)cc2)cc1] | 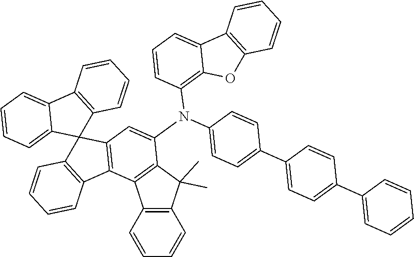 [SMILES: CC1(C)c2ccccc2-c2c3c(cc(N(c4ccc(-c5ccc(-c6ccccc6)cc5)cc4)c4cccc5c4oc4ccccc45)c21)C1(c2ccccc2-c2ccccc21)c1ccccc1-3] | 54% |

### [Preparation Example 3] Preparation of Compound C-1

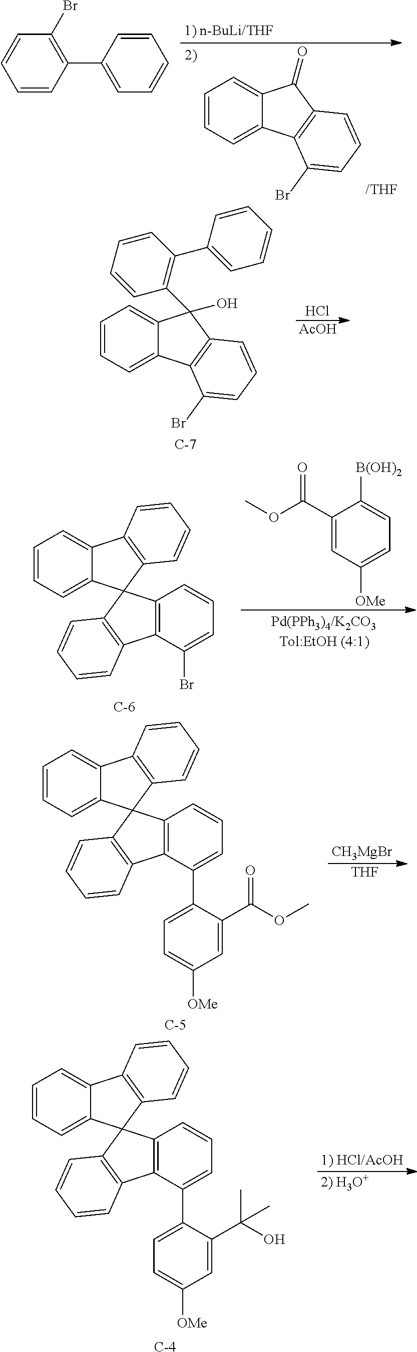

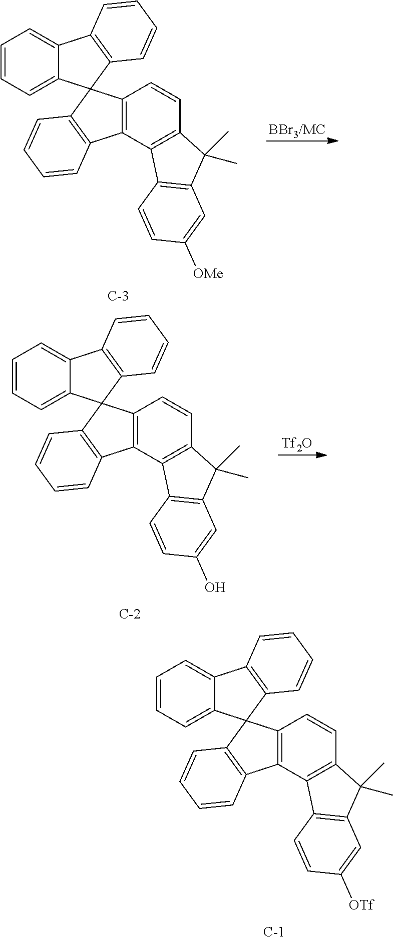

Preparation of Compound C-6

A compound C-6 was prepared in the same manner as that of Preparation Example 1, except that 2-bromo-1,1′-biphenyl (38.00 mmol) was used instead of 2-bromo-4′-methoxy-1,1′-biphenyl as a starting material.

Preparation of Compound C-5

10 g (23.51 mmol) of the compound C-6 was dissolved in 100 mL of toluene, 5.92 g (28.21 mmol) of 4-methoxy-2-(methoxycarbonyl)phenyl)boronic acid, 1.36 g (1.18 mmol) of Pd(PPh₃)₄, and 8.12 g (58.78 mmol) of K₂CO₃ were added, and stirring was performed. 25 mL of ethanol and 25 mL of water were added thereto, and the mixed solution was refluxed at 130° C. for 24 hours. After the reaction was completed, extraction was performed using MC and H₂O, the solvent was removed, and then purification was performed by column chromatography with Hex and MC, thereby obtaining 7.91 g (70%) of a target compound C-5.

Preparation of Compound C-4

10 g (20.81 mmol) of the compound C-5 was dissolved in 110 mL of THF, and then the temperature was lowered to −78° C. 16 mL (47.86 mmol) of methylmagnesium bromide (3.0 M solution in diethyl ether) was slowly added dropwise thereto, and stirring was performed at the same temperature for 30 minutes. After stirring was completed, the temperature of the reaction solution was slowly raised to room temperature, and stirring was additionally performed at room temperature for 24 hours. After the reaction was completed, extraction was performed using a sodium thiosulfate (Na₂S2O₃) solution and MC, the solvent was removed, and recrystallization purification was performed with Hex and EA, thereby obtaining 5.60 g (56%) of a target compound C-4.

Preparation of Compound C-3

10 g (20.81 mmol) of the compound C-4 was dissolved in 120 mL of AcOH, 50 mL of HCl was added thereto, and stirring was performed under reflux at 130° C. for 24 hours. After the reaction was completed, the reaction solution was cooled to room temperature, the produced solid was filtered, and washing was performed with a sodium bicarbonate (NaHCO₃) aqueous solution. Thereafter, washing was performed with water, ethanol, Hex, and EA, and then recrystallization was performed with Hex/EA, thereby obtaining 6.07 g (63%) of a target compound C-3.

Preparation of Compound C-2

10 g (21.62 mmol) of the compound C-3 was dissolved in 110 mL of MC, and then the temperature was lowered to 0° C. 25 mL (25.94 mmol) of tribromoboron (1.0 M solution in MC) was slowly added dropwise thereto, and stirring was performed at the same temperature for 30 minutes. The temperature of the reaction solution was slowly raised to room temperature, and stirring was additionally performed at room temperature for 24 hours. After the reaction was completed, extraction was performed with MC and a NaHCO₃ aqueous solution, the MC layer was collected, the solvent was removed, and the produced solid was purified by column chromatography using MC and Hex, thereby obtaining 6.59 g (68%) of a target compound C-2.

Preparation of Compound C-1

10 g (22.30 mmol) of the compound C-2 was dissolved in 120 mL of THF, 3.1 mL (22.30 mmol) of TEA and 4.5 mL (26.76 mmol) of triflic anhydride were added, and then stirring was performed at room temperature for 24 hours. After the reaction was completed, extraction was performed using MC and a NaHCO₃ aqueous solution, the MC layer was collected, the solvent was removed, and the produced solid was purified by column chromatography using MC and Hex, thereby obtaining 8.93 g (69%) of a target compound C-1.

### [Example 3] Preparation of Compounds P5 and P6

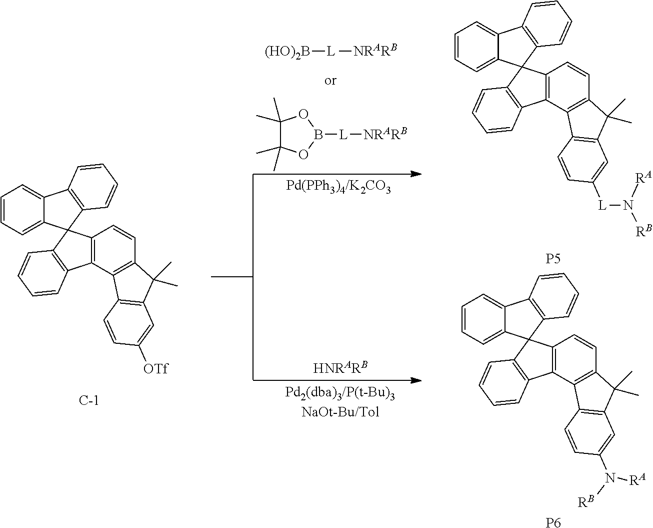

Preparation of Compound P5

A target compound P5 was obtained through a Suzuki coupling reaction using the compound C-1, a substituted boronic acid or a boronic ester compound, and a palladium catalyst.

Preparation of Compound P6

A target compound P6 was obtained through palladium-amination using the compound C-1, a secondary amine compound, and a palladium catalyst.

The structures of the substituted boronic acid, boronic ester, and secondary amine compound as the reactants and the structures and yields of the prepared compounds P5 and P6 are shown in Table 3.

TABLE 3

| Com- | | | |
|---|---|---|---|
| pound | | | |
| No. | Reactant | Product (P5, P6) | Yield |
| Com 23-1 | 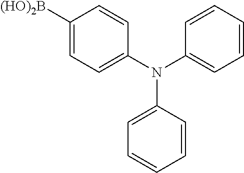 [SMILES: OB(O)c1ccc(N(c2ccccc2)c2ccccc2)cc1] | 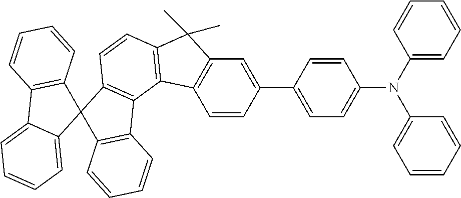 [SMILES: CC1(C)c2cc(-c3ccc(N(c4ccccc4)c4ccccc4)cc3)ccc2-c2c1ccc1c2-c2ccccc2C12c1ccccc1-c1ccccc12] | 81% |
| Com 23-2 | 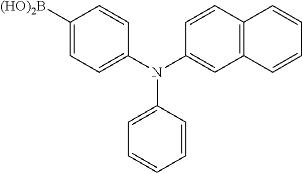 [SMILES: OB(O)c1ccc(N(c2ccccc2)c2ccc3ccccc3c2)cc1] | 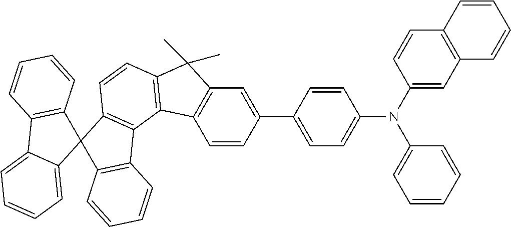 [SMILES: CC1(C)c2cc(-c3ccc(N(c4ccccc4)c4ccc5ccccc5c4)cc3)ccc2-c2c1ccc1c2-c2ccccc2C12c1ccccc1-c1ccccc12] | 78% |
| Com 23-3 | 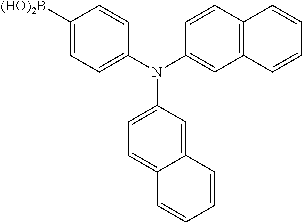 [SMILES: OB(O)c1ccc(N(c2ccc3ccccc3c2)c2ccc3ccccc3c2)cc1] | 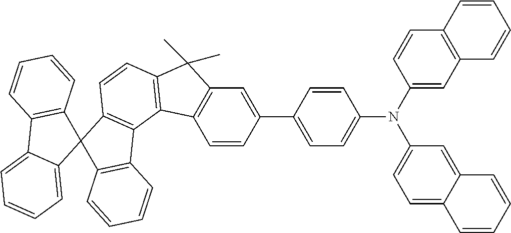 [SMILES: CC1(C)c2cc(-c3ccc(N(c4ccc5ccccc5c4)c4ccc5ccccc5c4)cc3)ccc2-c2c1ccc1c2-c2ccccc2C12c1ccccc1-c1ccccc12] | 81% |
| Com 23-4 | 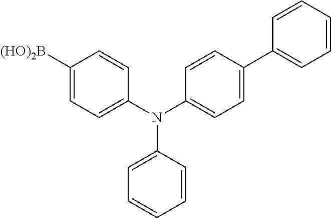 [SMILES: OB(O)c1ccc(N(c2ccccc2)c2ccc(-c3ccccc3)cc2)cc1] | 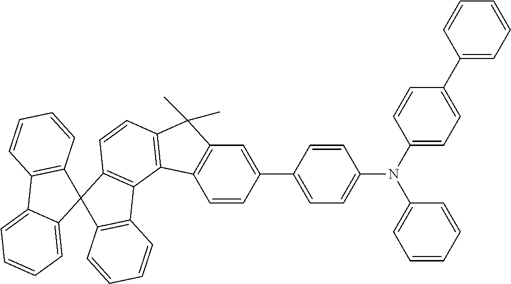 [SMILES: CC1(C)c2cc(-c3ccc(N(c4ccccc4)c4ccc(-c5ccccc5)cc4)cc3)ccc2-c2c1ccc1c2-c2ccccc2C12c1ccccc1-c1ccccc12] | 89% |
| Com 23-5 | 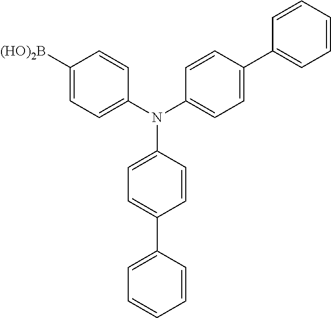 [SMILES: OB(O)c1ccc(N(c2ccc(-c3ccccc3)cc2)c2ccc(-c3ccccc3)cc2)cc1] | 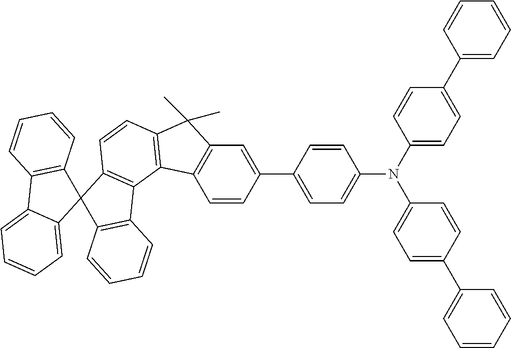 [SMILES: CC1(C)c2cc(-c3ccc(N(c4ccc(-c5ccccc5)cc4)c4ccc(-c5ccccc5)cc4)cc3)ccc2-c2c1ccc1c2-c2ccccc2C12c1ccccc1-c1ccccc12] | 74% |
| Com 23-6 | 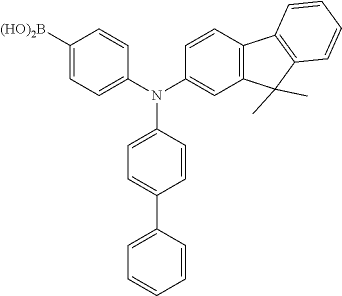 [SMILES: CC1(C)c2ccccc2-c2ccc(N(c3ccc(B(O)O)cc3)c3ccc(-c4ccccc4)cc3)cc21] | 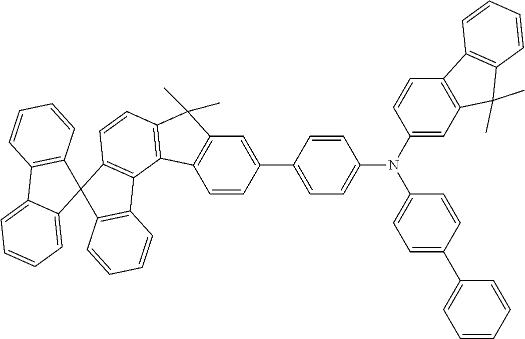 [SMILES: CC1(C)c2ccccc2-c2ccc(N(c3ccc(-c4ccccc4)cc3)c3ccc(-c4ccc5c(c4)C(C)(C)c4ccc6c(c4-5)-c4ccccc4C64c5ccccc5-c5ccccc54)cc3)cc21] | 76% |
| Com 23-7 | 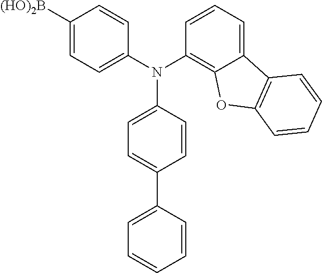 [SMILES: OB(O)c1ccc(N(c2ccc(-c3ccccc3)cc2)c2cccc3c2oc2ccccc23)cc1] | 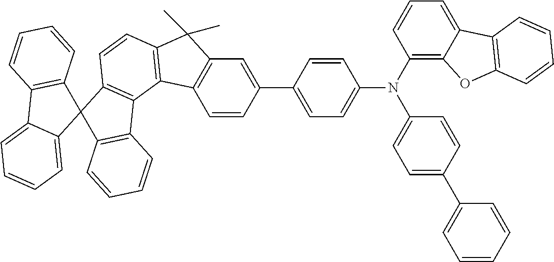 [SMILES: CC1(C)c2cc(-c3ccc(N(c4ccc(-c5ccccc5)cc4)c4cccc5c4oc4ccccc45)cc3)ccc2-c2c1ccc1c2-c2ccccc2C12c1ccccc1-c1ccccc12] | 75% |
| Com 23-8 | 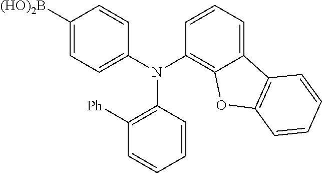 [SMILES: OB(O)c1ccc(N(c2ccccc2-c2ccccc2)c2cccc3c2oc2ccccc23)cc1] | 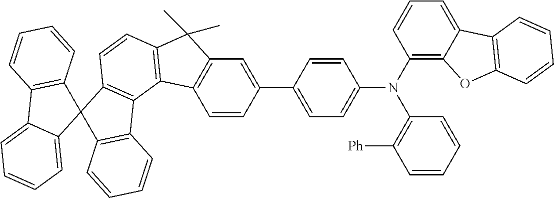 [SMILES: CC1(C)c2cc(-c3ccc(N(c4ccccc4-c4ccccc4)c4cccc5c4oc4ccccc45)cc3)ccc2-c2c1ccc1c2-c2ccccc2C12c1ccccc1-c1ccccc12] | 74% |
| Com 23-9 | 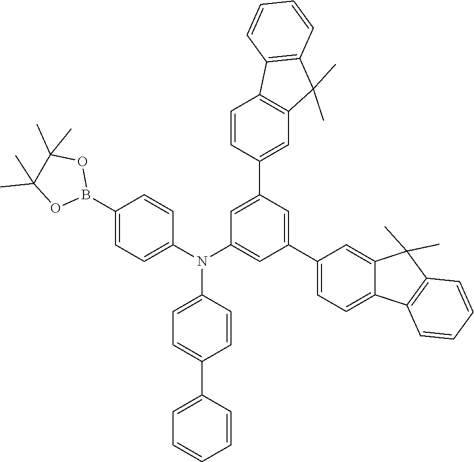 [SMILES: CC1(C)c2ccccc2-c2ccc(-c3cc(-c4ccc5c(c4)C(C)(C)c4ccccc4-5)cc(N(c4ccc(B5OC(C)(C)C(C)(C)O5)cc4)c4ccc(-c5ccccc5)cc4)c3)cc21] | 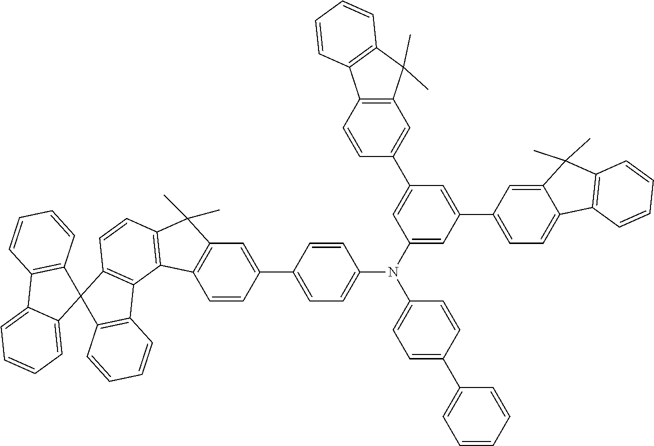 [SMILES: CC1(C)c2ccccc2-c2ccc(-c3cc(-c4ccc5c(c4)C(C)(C)c4ccccc4-5)cc(N(c4ccc(-c5ccccc5)cc4)c4ccc(-c5ccc6c(c5)C(C)(C)c5ccc7c(c5-6)-c5ccccc5C75c6ccccc6-c6ccccc65)cc4)c3)cc21] | 58% |
| Com 23-10 | 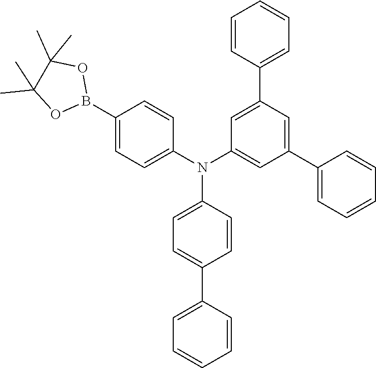 [SMILES: CC1(C)OB(c2ccc(N(c3ccc(-c4ccccc4)cc3)c3cc(-c4ccccc4)cc(-c4ccccc4)c3)cc2)OC1(C)C] | 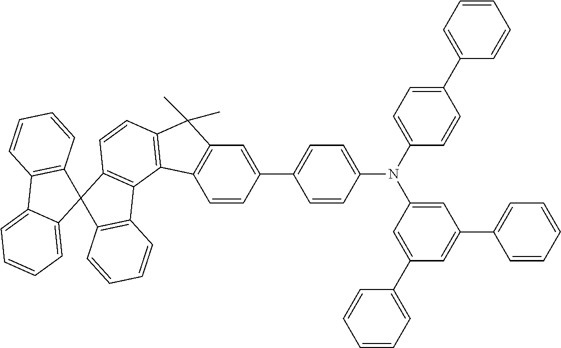 [SMILES: CC1(C)c2cc(-c3ccc(N(c4ccc(-c5ccccc5)cc4)c4cc(-c5ccccc5)cc(-c5ccccc5)c4)cc3)ccc2-c2c1ccc1c2-c2ccccc2C12c1ccccc1-c1ccccc12] | 72% |
| Com 23-11 | 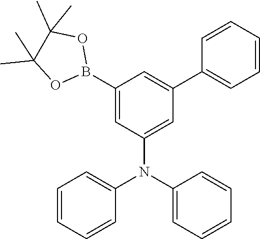 [SMILES: CC1(C)OB(c2cc(-c3ccccc3)cc(N(c3ccccc3)c3ccccc3)c2)OC1(C)C] | 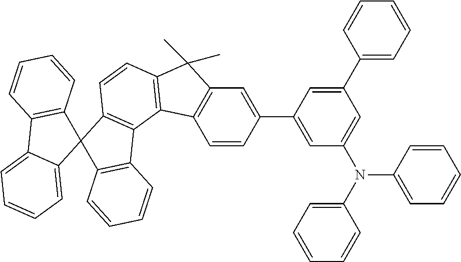 [SMILES: CC1(C)c2cc(-c3cc(-c4ccccc4)cc(N(c4ccccc4)c4ccccc4)c3)ccc2-c2c1ccc1c2-c2ccccc2C12c1ccccc1-c1ccccc12] | 92% |
| Com 23-12 | 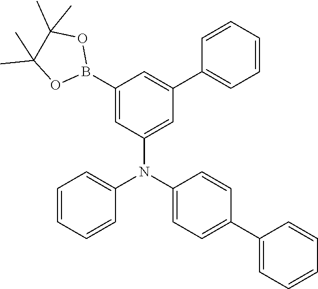 [SMILES: CC1(C)OB(c2cc(-c3ccccc3)cc(N(c3ccccc3)c3ccc(-c4ccccc4)cc3)c2)OC1(C)C] | 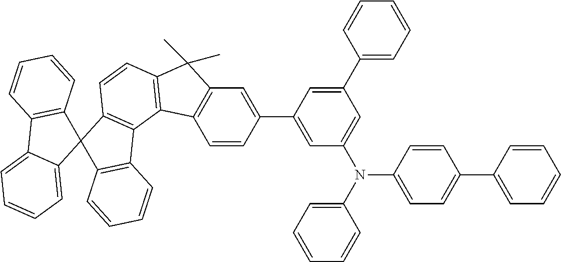 [SMILES: CC1(C)c2cc(-c3cc(-c4ccccc4)cc(N(c4ccccc4)c4ccc(-c5ccccc5)cc4)c3)ccc2-c2c1ccc1c2-c2ccccc2C12c1ccccc1-c1ccccc12] | 94% |
| Com 23-13 | 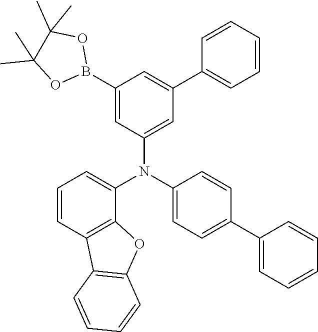 [SMILES: CC1(C)OB(c2cc(-c3ccccc3)cc(N(c3ccc(-c4ccccc4)cc3)c3cccc4c3oc3ccccc34)c2)OC1(C)C] | 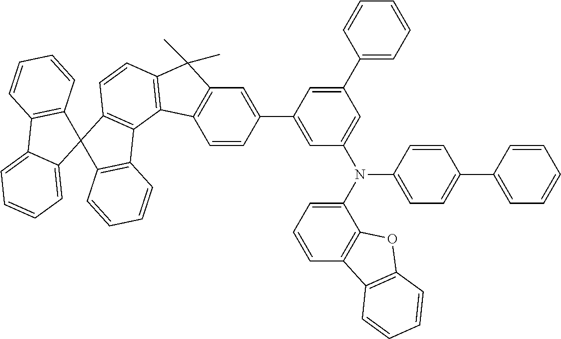 [SMILES: CC1(C)c2cc(-c3cc(-c4ccccc4)cc(N(c4ccc(-c5ccccc5)cc4)c4cccc5c4oc4ccccc45)c3)ccc2-c2c1ccc1c2-c2ccccc2C12c1ccccc1-c1ccccc12] | 84% |
| Com 23-14 | 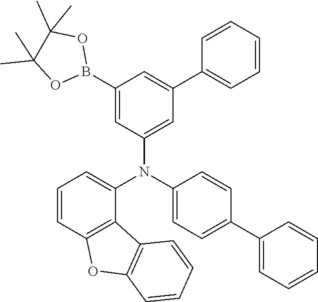 [SMILES: CC1(C)OB(c2cc(-c3ccccc3)cc(N(c3ccc(-c4ccccc4)cc3)c3cccc4oc5ccccc5c34)c2)OC1(C)C] | 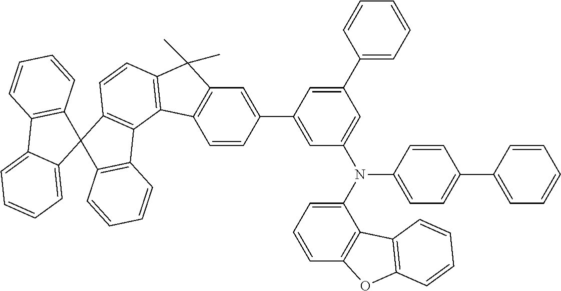 [SMILES: CC1(C)c2cc(-c3cc(-c4ccccc4)cc(N(c4ccc(-c5ccccc5)cc4)c4cccc5oc6ccccc6c45)c3)ccc2-c2c1ccc1c2-c2ccccc2C12c1ccccc1-c1ccccc12] | 88% |
| Com 23-15 | 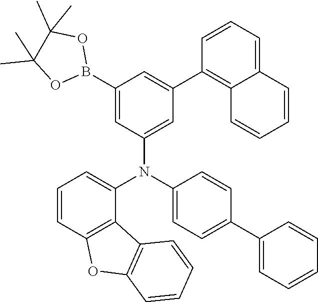 [SMILES: CC1(C)OB(c2cc(-c3cccc4ccccc34)cc(N(c3ccc(-c4ccccc4)cc3)c3cccc4oc5ccccc5c34)c2)OC1(C)C] | 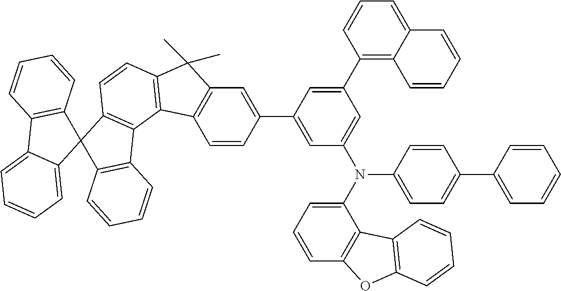 [SMILES: CC1(C)c2cc(-c3cc(-c4cccc5ccccc45)cc(N(c4ccc(-c5ccccc5)cc4)c4cccc5oc6ccccc6c45)c3)ccc2-c2c1ccc1c2-c2ccccc2C12c1ccccc1-c1ccccc12] | 78% |
| Com 23-16 | 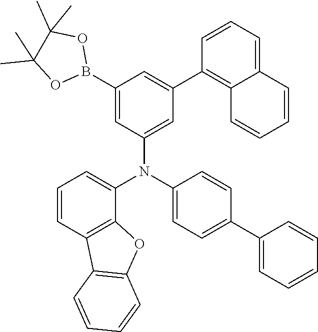 [SMILES: CC1(C)OB(c2cc(-c3cccc4ccccc34)cc(N(c3ccc(-c4ccccc4)cc3)c3cccc4c3oc3ccccc34)c2)OC1(C)C] | 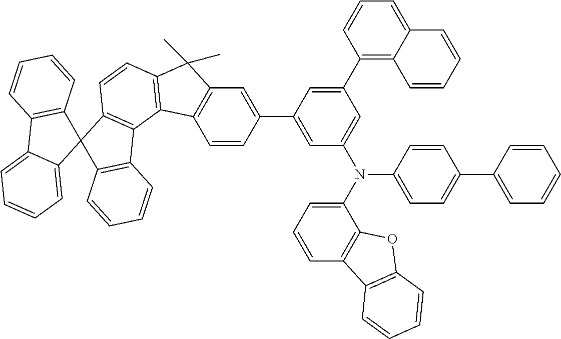 [SMILES: CC1(C)c2cc(-c3cc(-c4cccc5ccccc45)cc(N(c4ccc(-c5ccccc5)cc4)c4cccc5c4oc4ccccc45)c3)ccc2-c2c1ccc1c2-c2ccccc2C12c1ccccc1-c1ccccc12] | 71% |
| Com 23-17 | 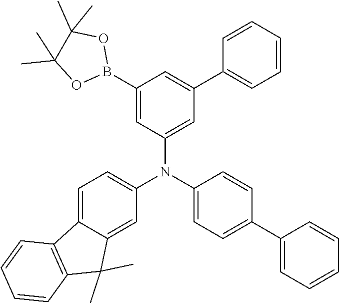 [SMILES: CC1(C)c2ccccc2-c2ccc(N(c3ccc(-c4ccccc4)cc3)c3cc(B4OC(C)(C)C(C)(C)O4)cc(-c4ccccc4)c3)cc21] | 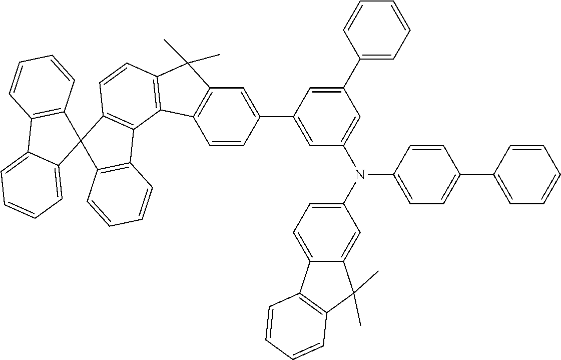 [SMILES: CC1(C)c2ccccc2-c2ccc(N(c3ccc(-c4ccccc4)cc3)c3cc(-c4ccccc4)cc(-c4ccc5c(c4)C(C)(C)c4ccc6c(c4-5)-c4ccccc4C64c5ccccc5-c5ccccc54)c3)cc21] | 78% |
| Com 23-18 | 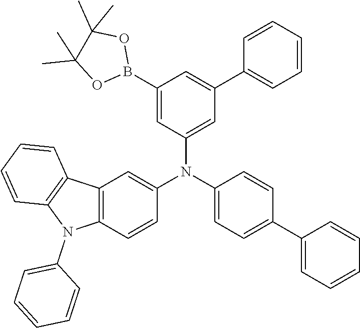 [SMILES: CC1(C)OB(c2cc(-c3ccccc3)cc(N(c3ccc(-c4ccccc4)cc3)c3ccc4c(c3)c3ccccc3n4-c3ccccc3)c2)OC1(C)C] | 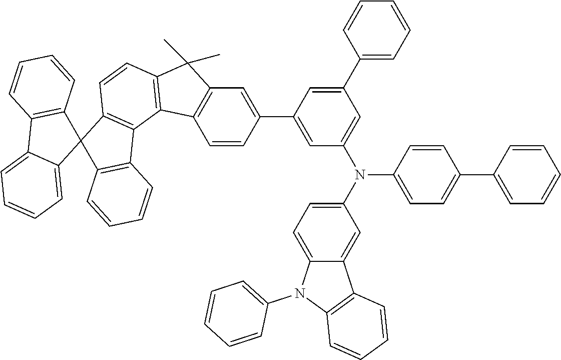 [SMILES: CC1(C)c2cc(-c3cc(-c4ccccc4)cc(N(c4ccc(-c5ccccc5)cc4)c4ccc5c(c4)c4ccccc4n5-c4ccccc4)c3)ccc2-c2c1ccc1c2-c2ccccc2C12c1ccccc1-c1ccccc12] | 74% |
| Com 23-19 | 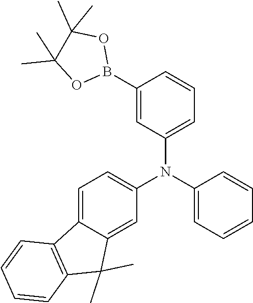 [SMILES: CC1(C)c2ccccc2-c2ccc(N(c3ccccc3)c3cccc(B4OC(C)(C)C(C)(C)O4)c3)cc21] | 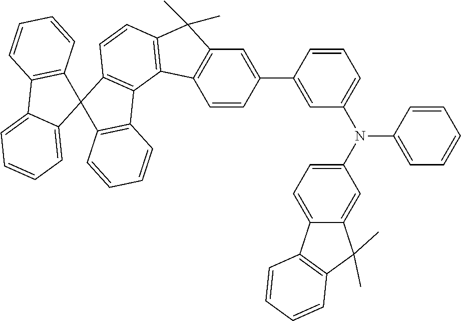 [SMILES: CC1(C)c2ccccc2-c2ccc(N(c3ccccc3)c3cccc(-c4ccc5c(c4)C(C)(C)c4ccc6c(c4-5)-c4ccccc4C64c5ccccc5-c5ccccc54)c3)cc21] | 65% |
| Com 23-20 | 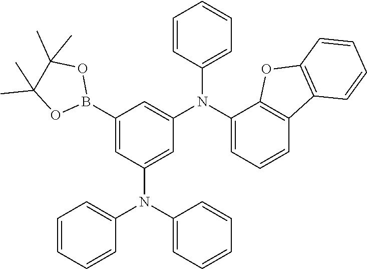 [SMILES: CC1(C)OB(c2cc(N(c3ccccc3)c3ccccc3)cc(N(c3ccccc3)c3cccc4c3oc3ccccc34)c2)OC1(C)C] | 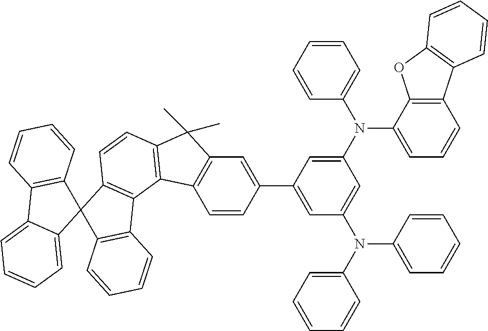 [SMILES: CC1(C)c2cc(-c3cc(N(c4ccccc4)c4ccccc4)cc(N(c4ccccc4)c4cccc5c4oc4ccccc45)c3)ccc2-c2c1ccc1c2-c2ccccc2C12c1ccccc1-c1ccccc12] | 74% |
| Com 23-21 | 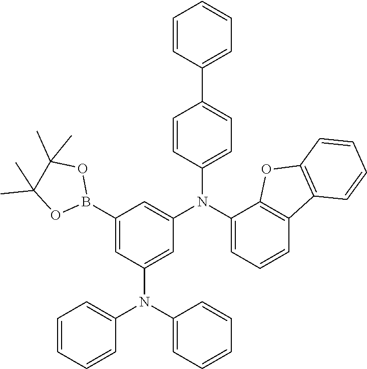 [SMILES: CC1(C)OB(c2cc(N(c3ccccc3)c3ccccc3)cc(N(c3ccc(-c4ccccc4)cc3)c3cccc4c3oc3ccccc34)c2)OC1(C)C] | 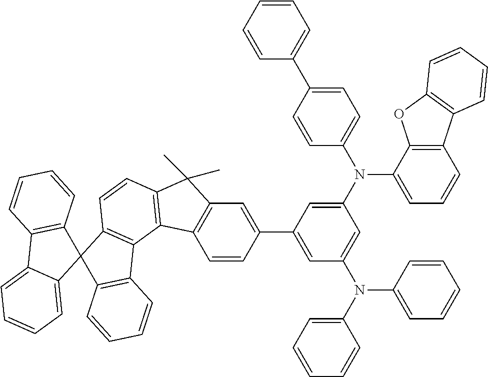 [SMILES: CC1(C)c2cc(-c3cc(N(c4ccccc4)c4ccccc4)cc(N(c4ccc(-c5ccccc5)cc4)c4cccc5c4oc4ccccc45)c3)ccc2-c2c1ccc1c2-c2ccccc2C12c1ccccc1-c1ccccc12] | 75% |
| Com 23-22 | 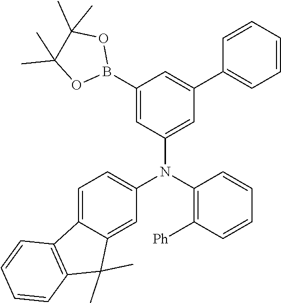 [SMILES: CC1(C)c2ccccc2-c2ccc(N(c3cc(B4OC(C)(C)C(C)(C)O4)cc(-c4ccccc4)c3)c3ccccc3-c3ccccc3)cc21] | 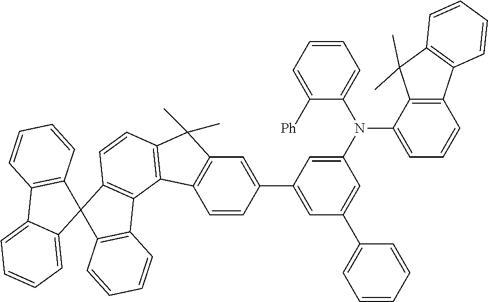 [SMILES: CC1(C)c2cc(-c3cc(-c4ccccc4)cc(N(c4ccccc4-c4ccccc4)c4cccc5c4C(C)(C)c4ccccc4-5)c3)ccc2-c2c1ccc1c2-c2ccccc2C12c1ccccc1-c1ccccc12] | 72% |
| Com 23-23 | 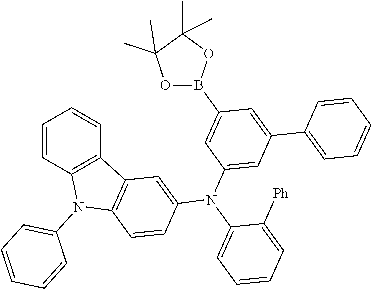 [SMILES: CC1(C)OB(c2cc(-c3ccccc3)cc(N(c3ccc4c(c3)c3ccccc3n4-c3ccccc3)c3ccccc3-c3ccccc3)c2)OC1(C)C] | 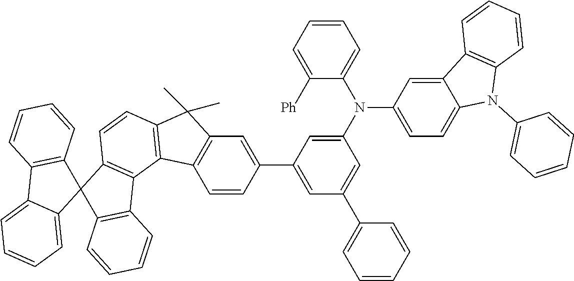 [SMILES: CC1(C)c2cc(-c3cc(-c4ccccc4)cc(N(c4ccc5c(c4)c4ccccc4n5-c4ccccc4)c4ccccc4-c4ccccc4)c3)ccc2-c2c1ccc1c2-c2ccccc2C12c1ccccc1-c1ccccc12] | 77% |
| Com 23-24 | 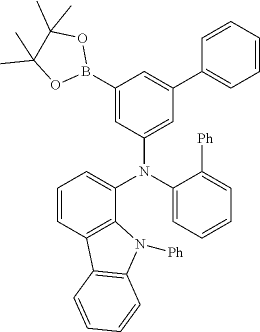 [SMILES: CC1(C)OB(c2cc(-c3ccccc3)cc(N(c3ccccc3-c3ccccc3)c3cccc4c5ccccc5n(-c5ccccc5)c34)c2)OC1(C)C] | 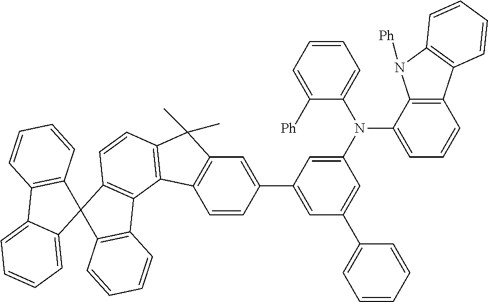 [SMILES: CC1(C)c2cc(-c3cc(-c4ccccc4)cc(N(c4ccccc4-c4ccccc4)c4cccc5c6ccccc6n(-c6ccccc6)c45)c3)ccc2-c2c1ccc1c2-c2ccccc2C12c1ccccc1-c1ccccc12] | 70% |
| Com 23-25 | 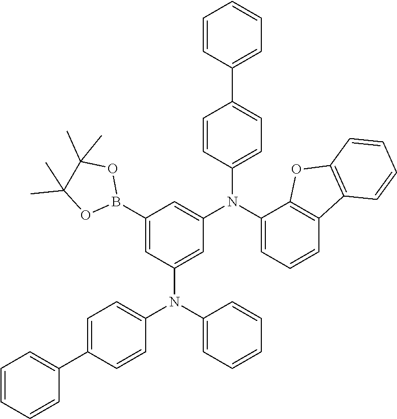 [SMILES: CC1(C)OB(c2cc(N(c3ccccc3)c3ccc(-c4ccccc4)cc3)cc(N(c3ccc(-c4ccccc4)cc3)c3cccc4c3oc3ccccc34)c2)OC1(C)C] | 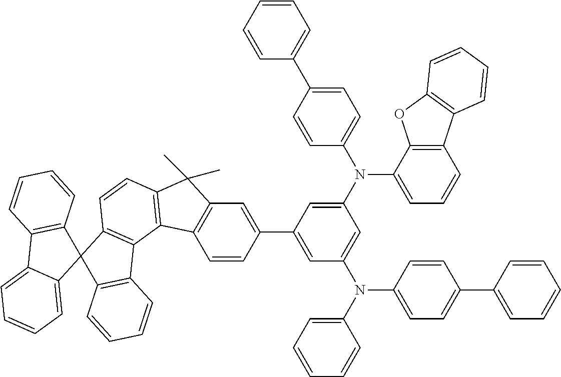 [SMILES: CC1(C)c2cc(-c3cc(N(c4ccccc4)c4ccc(-c5ccccc5)cc4)cc(N(c4ccc(-c5ccccc5)cc4)c4cccc5c4oc4ccccc45)c3)ccc2-c2c1ccc1c2-c2ccccc2C12c1ccccc1-c1ccccc12] | 72% |
| Com 23-26 | 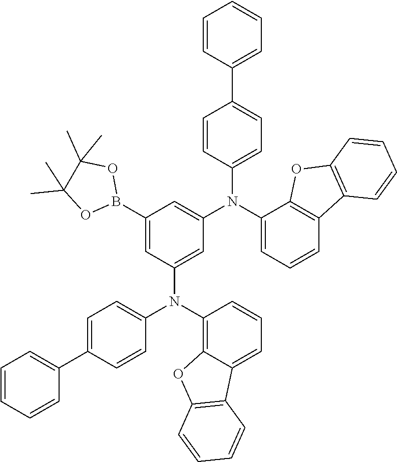 [SMILES: CC1(C)OB(c2cc(N(c3ccc(-c4ccccc4)cc3)c3cccc4c3oc3ccccc34)cc(N(c3ccc(-c4ccccc4)cc3)c3cccc4c3oc3ccccc34)c2)OC1(C)C] | 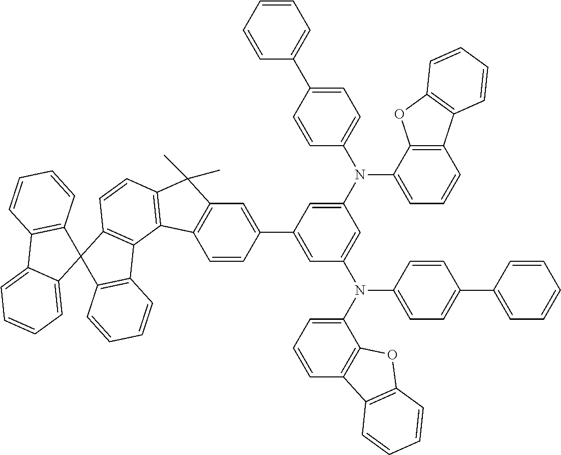 [SMILES: CC1(C)c2cc(-c3cc(N(c4ccc(-c5ccccc5)cc4)c4cccc5c4oc4ccccc45)cc(N(c4ccc(-c5ccccc5)cc4)c4cccc5c4oc4ccccc45)c3)ccc2-c2c1ccc1c2-c2ccccc2C12c1ccccc1-c1ccccc12] | 63% |
| Com 23-27 | 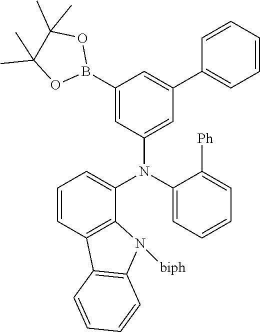 | 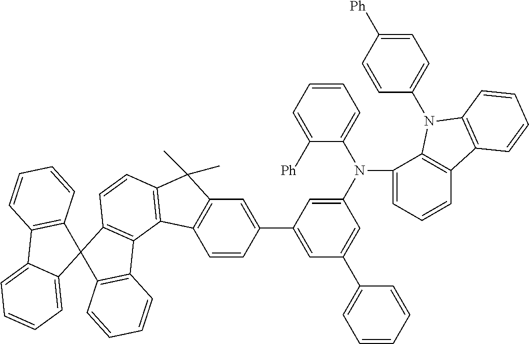 [SMILES: CC1(C)c2cc(-c3cc(-c4ccccc4)cc(N(c4ccccc4-c4ccccc4)c4cccc5c6ccccc6n(-c6ccc(-c7ccccc7)cc6)c45)c3)ccc2-c2c1ccc1c2-c2ccccc2C12c1ccccc1-c1ccccc12] | 60% |
| Com 23-28 | 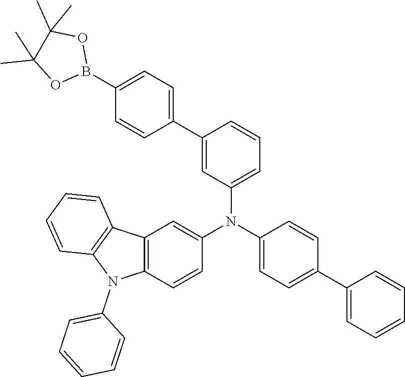 [SMILES: CC1(C)OB(c2ccc(-c3cccc(N(c4ccc(-c5ccccc5)cc4)c4ccc5c(c4)c4ccccc4n5-c4ccccc4)c3)cc2)OC1(C)C] | 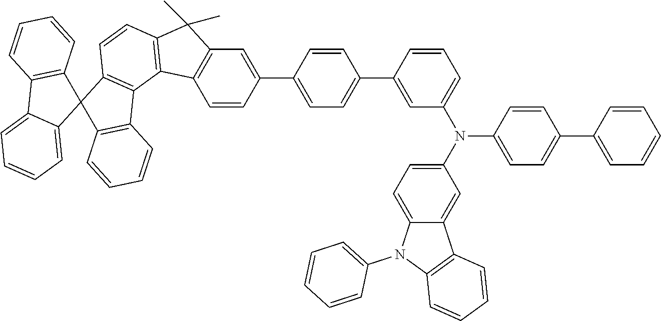 [SMILES: CC1(C)c2cc(-c3ccc(-c4cccc(N(c5ccc(-c6ccccc6)cc5)c5ccc6c(c5)c5ccccc5n6-c5ccccc5)c4)cc3)ccc2-c2c1ccc1c2-c2ccccc2C12c1ccccc1-c1ccccc12] | 52% |
| Com 23-29 | 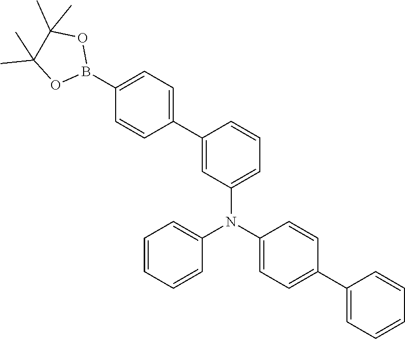 [SMILES: CC1(C)OB(c2ccc(-c3cccc(N(c4ccccc4)c4ccc(-c5ccccc5)cc4)c3)cc2)OC1(C)C] | 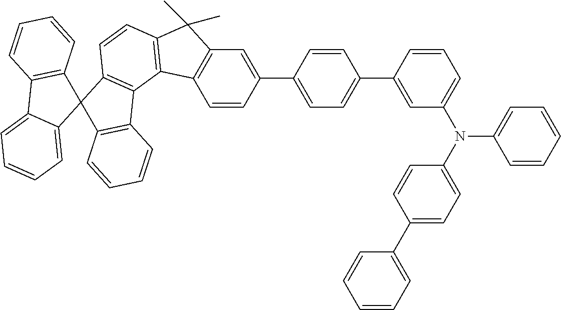 [SMILES: CC1(C)c2cc(-c3ccc(-c4cccc(N(c5ccccc5)c5ccc(-c6ccccc6)cc5)c4)cc3)ccc2-c2c1ccc1c2-c2ccccc2C12c1ccccc1-c1ccccc12] | 54% |
| Com 23-30 | 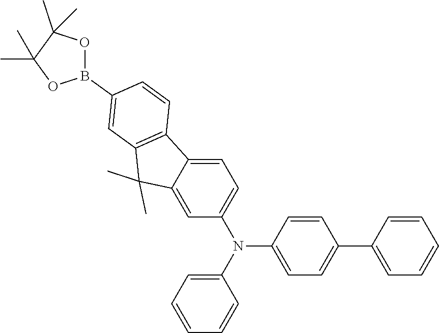 [SMILES: CC1(C)c2cc(B3OC(C)(C)C(C)(C)O3)ccc2-c2ccc(N(c3ccccc3)c3ccc(-c4ccccc4)cc3)cc21] | 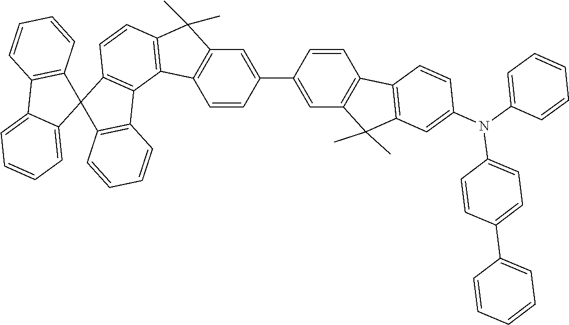 [SMILES: CC1(C)c2cc(-c3ccc4c(c3)C(C)(C)c3ccc5c(c3-4)-c3ccccc3C53c4ccccc4-c4ccccc43)ccc2-c2ccc(N(c3ccccc3)c3ccc(-c4ccccc4)cc3)cc21] | 57% |
| Com 23-31 | 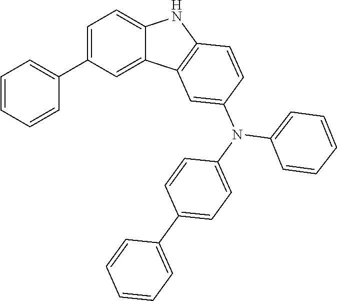 [SMILES: c1ccc(-c2ccc(N(c3ccccc3)c3ccc4[nH]c5ccc(-c6ccccc6)cc5c4c3)cc2)cc1] | 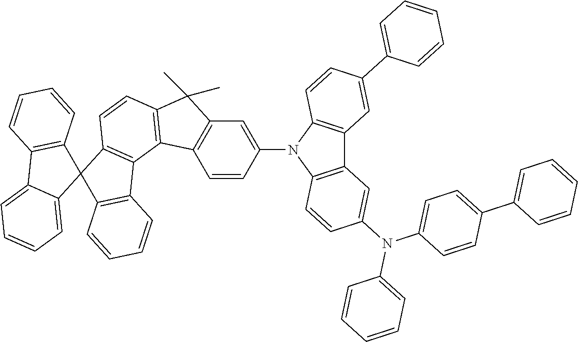 [SMILES: CC1(C)c2cc(-n3c4ccc(-c5ccccc5)cc4c4cc(N(c5ccccc5)c5ccc(-c6ccccc6)cc5)ccc43)ccc2-c2c1ccc1c2-c2ccccc2C12c1ccccc1-c1ccccc12] | 71% |
| Com 23-32 | 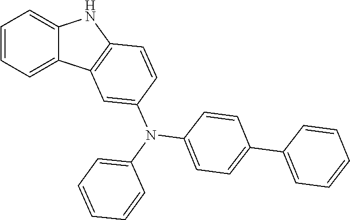 [SMILES: c1ccc(-c2ccc(N(c3ccccc3)c3ccc4[nH]c5ccccc5c4c3)cc2)cc1] | 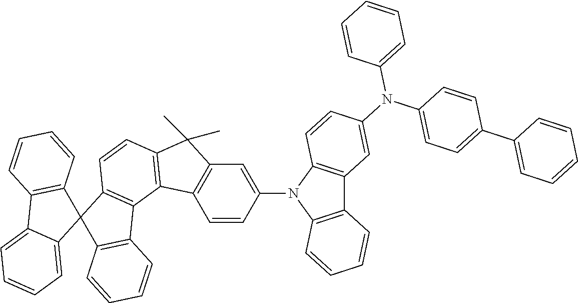 [SMILES: CC1(C)c2cc(-n3c4ccccc4c4cc(N(c5ccccc5)c5ccc(-c6ccccc6)cc5)ccc43)ccc2-c2c1ccc1c2-c2ccccc2C12c1ccccc1-c1ccccc12] | 67% |
| Com 23-33 | 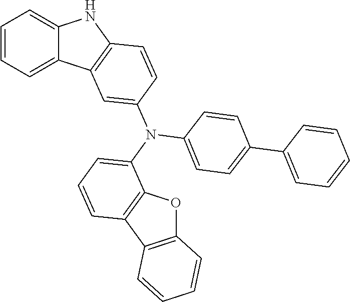 [SMILES: c1ccc(-c2ccc(N(c3ccc4[nH]c5ccccc5c4c3)c3cccc4c3oc3ccccc34)cc2)cc1] | 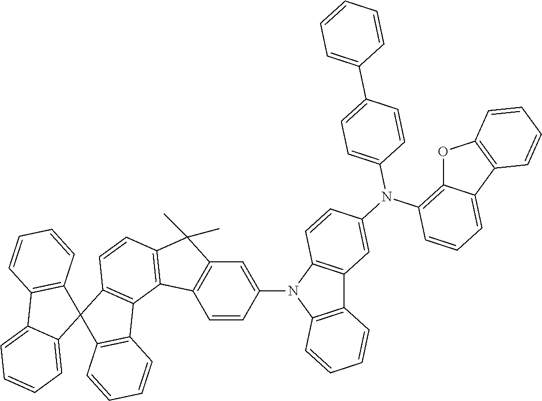 [SMILES: CC1(C)c2cc(-n3c4ccccc4c4cc(N(c5ccc(-c6ccccc6)cc5)c5cccc6c5oc5ccccc56)ccc43)ccc2-c2c1ccc1c2-c2ccccc2C12c1ccccc1-c1ccccc12] | 65% |
| Com 23-34 | 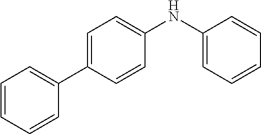 [SMILES: c1ccc(Nc2ccc(-c3ccccc3)cc2)cc1] | 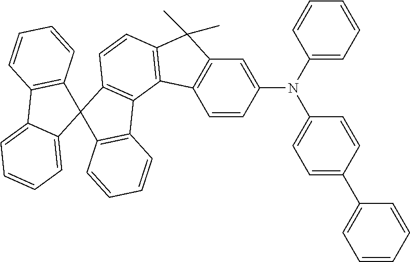 [SMILES: CC1(C)c2cc(N(c3ccccc3)c3ccc(-c4ccccc4)cc3)ccc2-c2c1ccc1c2-c2ccccc2C12c1ccccc1-c1ccccc12] | 81% |
| Com 23-35 | 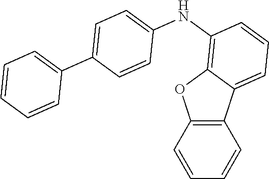 [SMILES: c1ccc(-c2ccc(Nc3cccc4c3oc3ccccc34)cc2)cc1] | 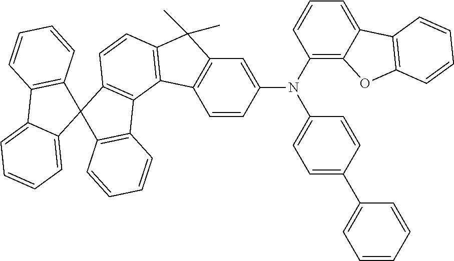 [SMILES: CC1(C)c2cc(N(c3ccc(-c4ccccc4)cc3)c3cccc4c3oc3ccccc34)ccc2-c2c1ccc1c2-c2ccccc2C12c1ccccc1-c1ccccc12] | 85% |
| Com 23-36 | 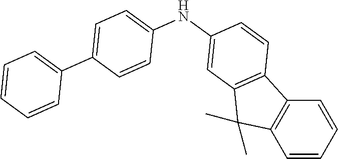 [SMILES: CC1(C)c2ccccc2-c2ccc(Nc3ccc(-c4ccccc4)cc3)cc21] | 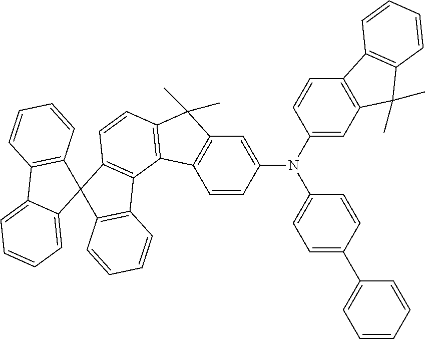 [SMILES: CC1(C)c2ccccc2-c2ccc(N(c3ccc(-c4ccccc4)cc3)c3ccc4c(c3)C(C)(C)c3ccc5c(c3-4)-c3ccccc3C53c4ccccc4-c4ccccc43)cc21] | 82% |
| Com 23-37 | 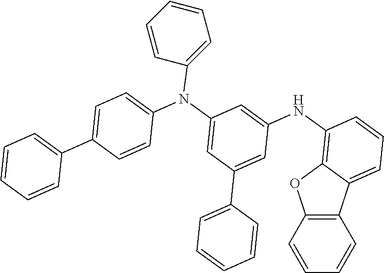 [SMILES: c1ccc(-c2ccc(N(c3ccccc3)c3cc(Nc4cccc5c4oc4ccccc45)cc(-c4ccccc4)c3)cc2)cc1] | 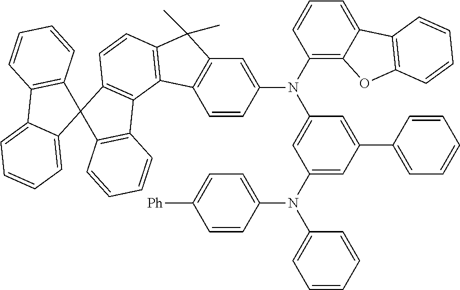 [SMILES: CC1(C)c2cc(N(c3cc(-c4ccccc4)cc(N(c4ccccc4)c4ccc(-c5ccccc5)cc4)c3)c3cccc4c3oc3ccccc34)ccc2-c2c1ccc1c2-c2ccccc2C12c1ccccc1-c1ccccc12] | 82% |
| Com 23-38 | 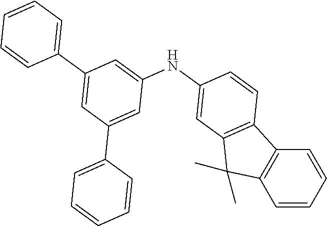 [SMILES: CC1(C)c2ccccc2-c2ccc(Nc3cc(-c4ccccc4)cc(-c4ccccc4)c3)cc21] | 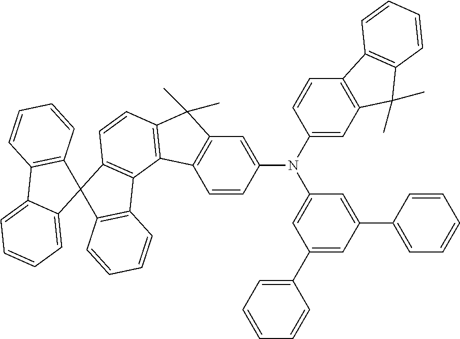 [SMILES: CC1(C)c2ccccc2-c2ccc(N(c3cc(-c4ccccc4)cc(-c4ccccc4)c3)c3ccc4c(c3)C(C)(C)c3ccc5c(c3-4)-c3ccccc3C53c4ccccc4-c4ccccc43)cc21] | 81% |
| Com 23-39 | 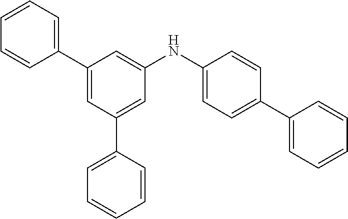 [SMILES: c1ccc(-c2ccc(Nc3cc(-c4ccccc4)cc(-c4ccccc4)c3)cc2)cc1] | 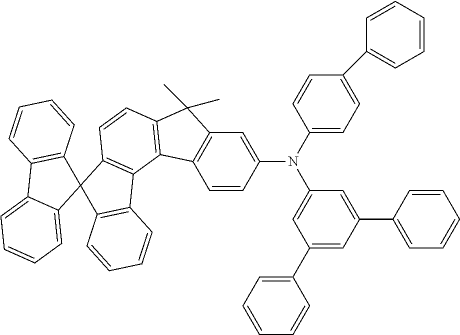 [SMILES: CC1(C)c2cc(N(c3ccc(-c4ccccc4)cc3)c3cc(-c4ccccc4)cc(-c4ccccc4)c3)ccc2-c2c1ccc1c2-c2ccccc2C12c1ccccc1-c1ccccc12] | 80% |
| Com 23-40 | 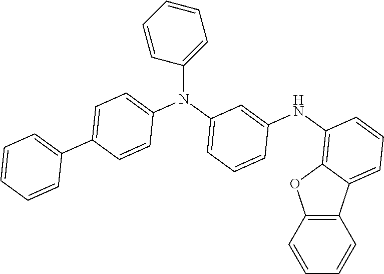 [SMILES: c1ccc(-c2ccc(N(c3ccccc3)c3cccc(Nc4cccc5c4oc4ccccc45)c3)cc2)cc1] | 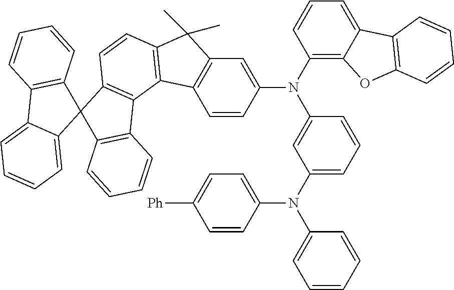 [SMILES: CC1(C)c2cc(N(c3cccc(N(c4ccccc4)c4ccc(-c5ccccc5)cc4)c3)c3cccc4c3oc3ccccc34)ccc2-c2c1ccc1c2-c2ccccc2C12c1ccccc1-c1ccccc12] | 70% |
| Com 23-41 | 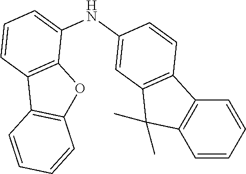 [SMILES: CC1(C)c2ccccc2-c2ccc(Nc3cccc4c3oc3ccccc34)cc21] | 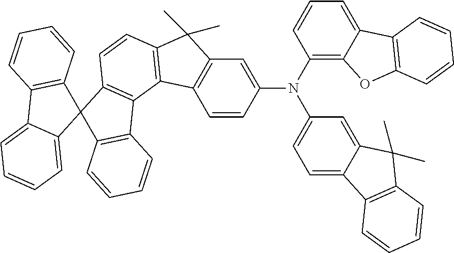 [SMILES: CC1(C)c2ccccc2-c2ccc(N(c3ccc4c(c3)C(C)(C)c3ccc5c(c3-4)-c3ccccc3C53c4ccccc4-c4ccccc43)c3cccc4c3oc3ccccc34)cc21] | 88% |
| Com 23-42 | 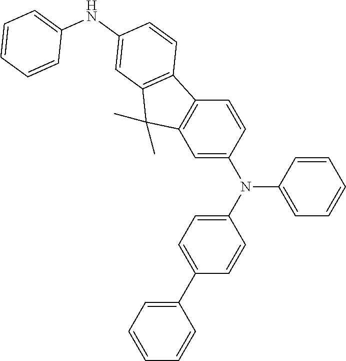 [SMILES: CC1(C)c2cc(Nc3ccccc3)ccc2-c2ccc(N(c3ccccc3)c3ccc(-c4ccccc4)cc3)cc21] | 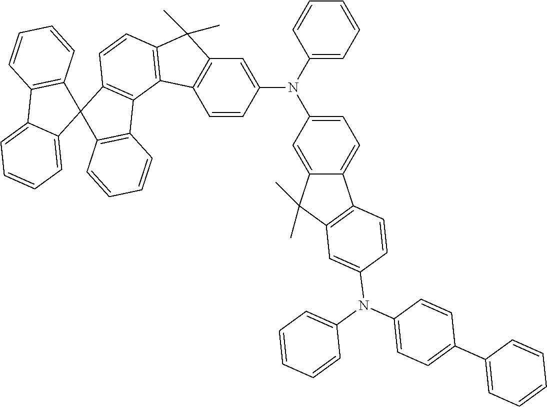 [SMILES: CC1(C)c2cc(N(c3ccccc3)c3ccc(-c4ccccc4)cc3)ccc2-c2ccc(N(c3ccccc3)c3ccc4c(c3)C(C)(C)c3ccc5c(c3-4)-c3ccccc3C53c4ccccc4-c4ccccc43)cc21] | 84% |
| Com 23-43 | 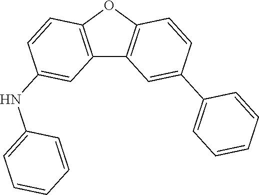 [SMILES: c1ccc(Nc2ccc3oc4ccc(-c5ccccc5)cc4c3c2)cc1] | 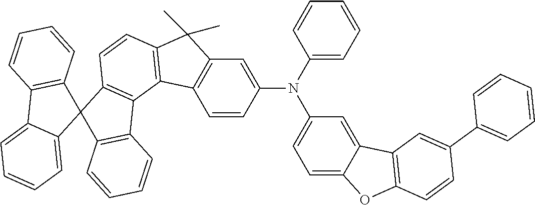 [SMILES: CC1(C)c2cc(N(c3ccccc3)c3ccc4oc5ccc(-c6ccccc6)cc5c4c3)ccc2-c2c1ccc1c2-c2ccccc2C12c1ccccc1-c1ccccc12] | 89% |
| Com 23-44 | 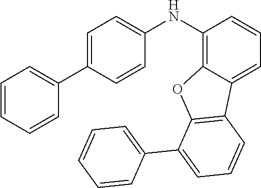 [SMILES: c1ccc(-c2ccc(Nc3cccc4c3oc3c(-c5ccccc5)cccc34)cc2)cc1] | 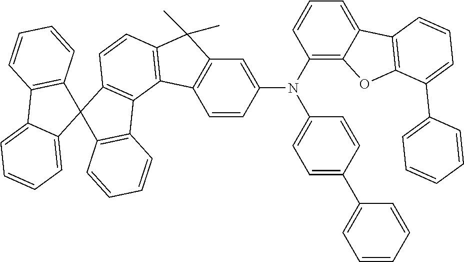 [SMILES: CC1(C)c2cc(N(c3ccc(-c4ccccc4)cc3)c3cccc4c3oc3c(-c5ccccc5)cccc34)ccc2-c2c1ccc1c2-c2ccccc2C12c1ccccc1-c1ccccc12] | 83% |
| Com 23-45 | 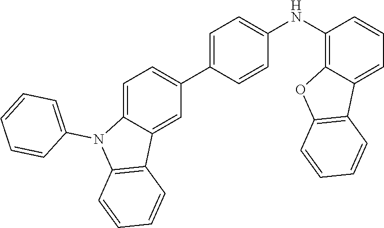 [SMILES: c1ccc(-n2c3ccccc3c3cc(-c4ccc(Nc5cccc6c5oc5ccccc56)cc4)ccc32)cc1] | 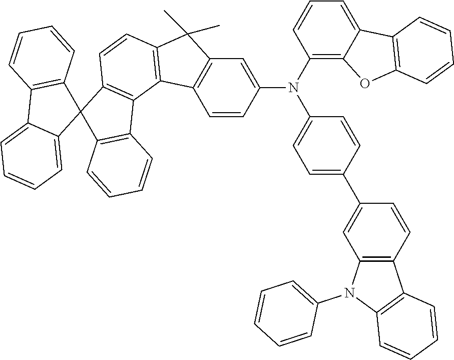 [SMILES: CC1(C)c2cc(N(c3ccc(-c4ccc5c6ccccc6n(-c6ccccc6)c5c4)cc3)c3cccc4c3oc3ccccc34)ccc2-c2c1ccc1c2-c2ccccc2C12c1ccccc1-c1ccccc12] | 75% |
| Com 23-46 | 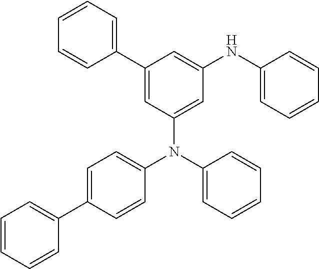 [SMILES: c1ccc(Nc2cc(-c3ccccc3)cc(N(c3ccccc3)c3ccc(-c4ccccc4)cc3)c2)cc1] | 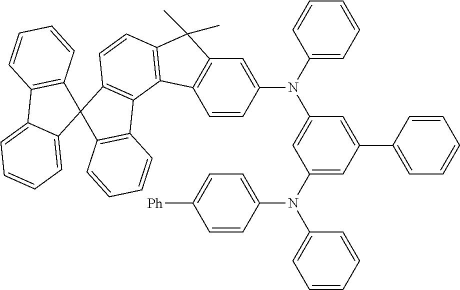 [SMILES: CC1(C)c2cc(N(c3ccccc3)c3cc(-c4ccccc4)cc(N(c4ccccc4)c4ccc(-c5ccccc5)cc4)c3)ccc2-c2c1ccc1c2-c2ccccc2C12c1ccccc1-c1ccccc12] | 65% |
| Com 23-47 | 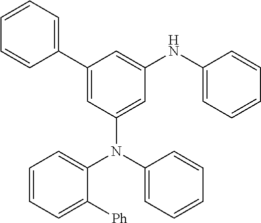 [SMILES: c1ccc(Nc2cc(-c3ccccc3)cc(N(c3ccccc3)c3ccccc3-c3ccccc3)c2)cc1] | 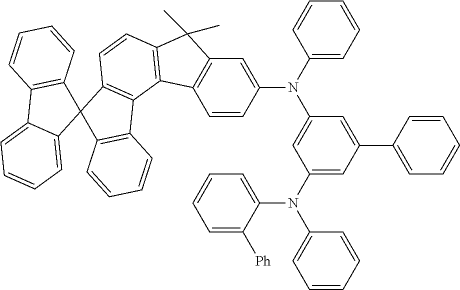 [SMILES: CC1(C)c2cc(N(c3ccccc3)c3cc(-c4ccccc4)cc(N(c4ccccc4)c4ccccc4-c4ccccc4)c3)ccc2-c2c1ccc1c2-c2ccccc2C12c1ccccc1-c1ccccc12] | 69% |
| Com 23-48 | 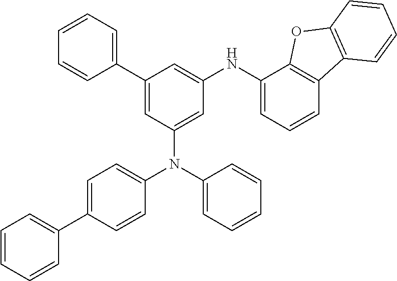 [SMILES: c1ccc(-c2ccc(N(c3ccccc3)c3cc(Nc4cccc5c4oc4ccccc45)cc(-c4ccccc4)c3)cc2)cc1] | 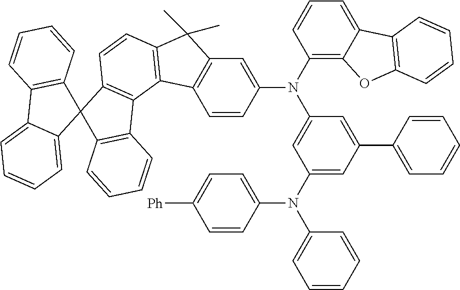 [SMILES: CC1(C)c2cc(N(c3cc(-c4ccccc4)cc(N(c4ccccc4)c4ccc(-c5ccccc5)cc4)c3)c3cccc4c3oc3ccccc34)ccc2-c2c1ccc1c2-c2ccccc2C12c1ccccc1-c1ccccc12] | 66% |
| Com 23-49 | 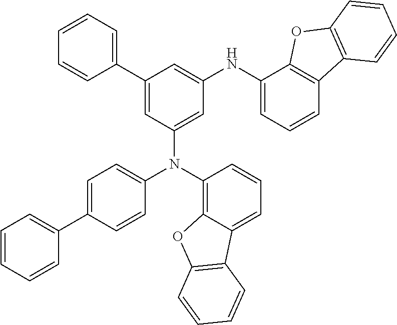 [SMILES: c1ccc(-c2ccc(N(c3cc(Nc4cccc5c4oc4ccccc45)cc(-c4ccccc4)c3)c3cccc4c3oc3ccccc34)cc2)cc1] | 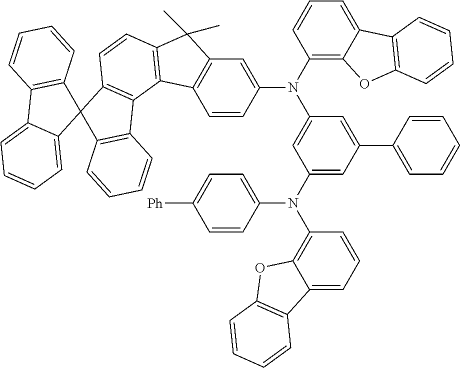 [SMILES: CC1(C)c2cc(N(c3cc(-c4ccccc4)cc(N(c4ccc(-c5ccccc5)cc4)c4cccc5c4oc4ccccc45)c3)c3cccc4c3oc3ccccc34)ccc2-c2c1ccc1c2-c2ccccc2C12c1ccccc1-c1ccccc12] | 54% |
| Com 23-50 | 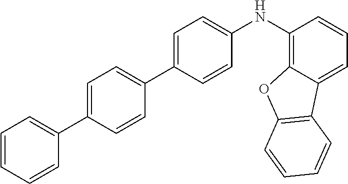 [SMILES: c1ccc(-c2ccc(-c3ccc(Nc4cccc5c4oc4ccccc45)cc3)cc2)cc1] | 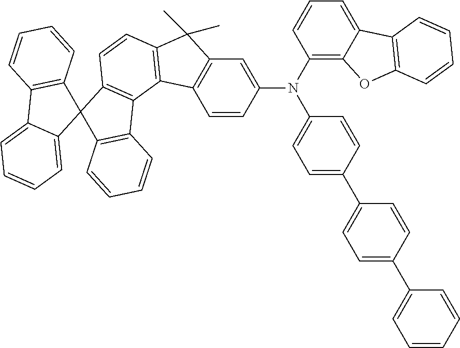 [SMILES: CC1(C)c2cc(N(c3ccc(-c4ccc(-c5ccccc5)cc4)cc3)c3cccc4c3oc3ccccc34)ccc2-c2c1ccc1c2-c2ccccc2C12c1ccccc1-c1ccccc12] | 80% |

### [Preparation Example 4] Preparation of Compound D-1

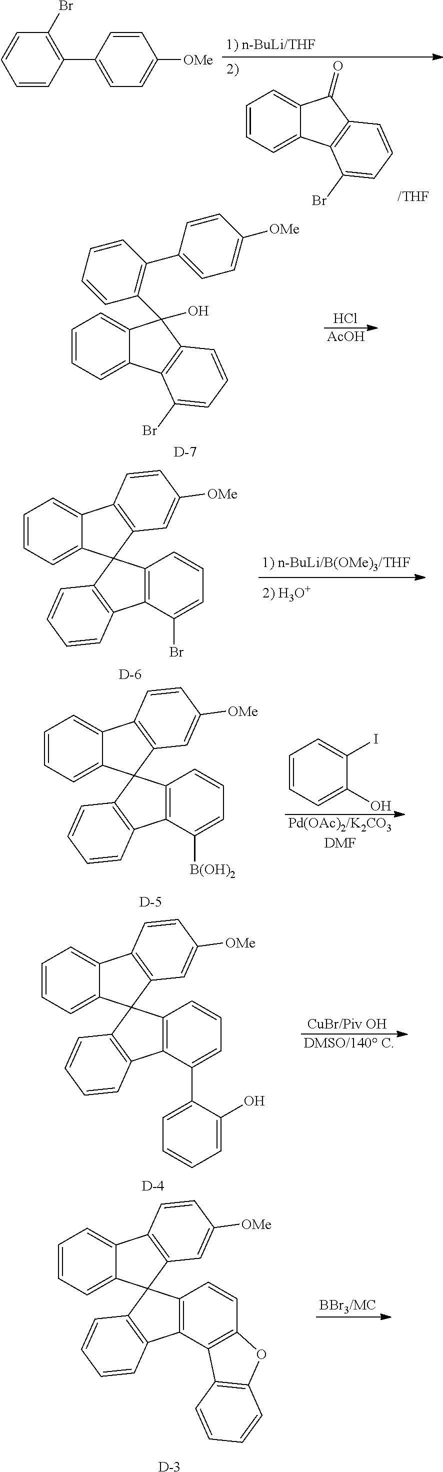

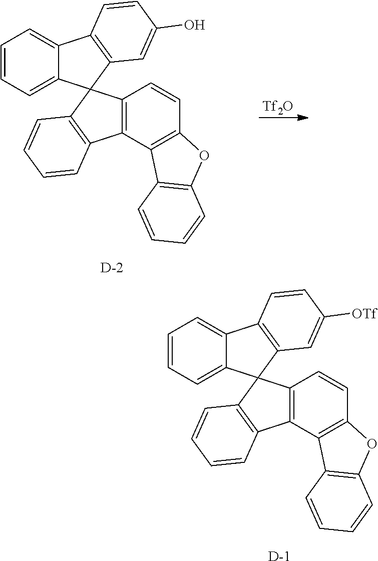

Preparation of Compound D-6

A compound D-6 was prepared in the same manner as that of Preparation Example 1.

Preparation of Compound D-5

10 g (23.51 mmol) of the compound D-6 was dissolved in 100 mL of THF, the temperature was lowered to −78° C., 18 mL (28.21 mmol) of n-butyllithium (1.6 M solution in hexane) was slowly added dropwise thereto, and stirring was performed at the same temperature for 30 minutes. While stirring was continued, the temperature was slowly raised to 0° C., and stirring was performed while slowly adding 3.2 mL (28.21 mmol) of trimethyl borate dropwise at 0° C. Stirring was performed at the same temperature for 2 hours, and stirring was performed for 18 hours while raising the temperature to room temperature. After the reaction was completed, the reaction was stopped with a 1 N HCl aqueous solution, extraction was performed using MC and H₂O, the solvent was removed, and recrystallization was performed with a small amount of THF and Hex, thereby obtaining 5.96 g (65%) of a target compound D-5.

Preparation of Compound D-4

10 g (25.62 mmol) of the compound D-5 and 6.76 g (30.75 mmol) of 2-iodophenol were dissolved in 110 mL of DMF, 0.86 g (3.84 mmol) of palladium acetate(II) and 10.62 g (76.86 mmol) of K₂CO₃ were added thereto under stirring, and stirring was performed under reflux for 24 hours. A sodium bicarbonate (NaHCO₃) aqueous solution was added to stop the reaction, extraction was performed using MC, the solvent was removed, and recrystallization purification was performed with DMF and Hex, thereby obtaining 9.66 g (86%) of a target compound D-4.

Preparation of Compound D-3

10 g (22.80 mmol) of the compound D-4 was dissolved in 120 mL of DMSO, 3.27 g (22.80 mmol) of copper(I) bromide (CuBr) and 5.12 mL (45.60 mmol) of pivalic acid were added, and stirring was performed under reflux at 140° C. for 24 hours. After the reaction was stopped, the reaction solution was cooled to room temperature, extraction was performed with a sodium bicarbonate (NaHCO₃) aqueous solution and MC, the solvent was removed, and the produced solid was washed with Hex and EA. Recrystallization was performed using DMF and Hex, thereby obtaining 6.37 g (64%) of a target compound D-3.

Preparation of Compound D-2

10 g (22.91 mmol) of the compound D-3 was dissolved in 110 mL of MC, and then the temperature was lowered to 0° C. 28 mL (27.49 mmol) of tribromoboron (1.0 M solution in MC) was slowly added dropwise thereto, and stirring was performed at the same temperature for 30 minutes. The temperature of the reaction solution was slowly raised to room temperature, and stirring was additionally performed at room temperature for 24 hours. After the reaction was completed, extraction was performed with MC and a NaHCO₃ aqueous solution, the MC layer was collected, the solvent was removed, and the produced solid was purified by column chromatography using MC and Hex, thereby obtaining 5.34 g (52%) of a target compound D-2.

Preparation of Compound D-1

10 g (22.29 mmol) of the compound D-2 was dissolved in 120 mL of THF, 3.1 mL (22.30 mmol) of TEA and 4.5 mL (26.76 mmol) of triflic anhydride were added, and then stirring was performed at room temperature for 24 hours. After the reaction was completed, extraction was performed using MC and a NaHCO₃ aqueous solution, the MC layer was collected, the solvent was removed, and the produced solid was purified by column chromatography using MC and Hex, thereby obtaining 8.41 g (65%) of a target compound D-1.

### [Example 4] Preparation of Compounds P7 and P8

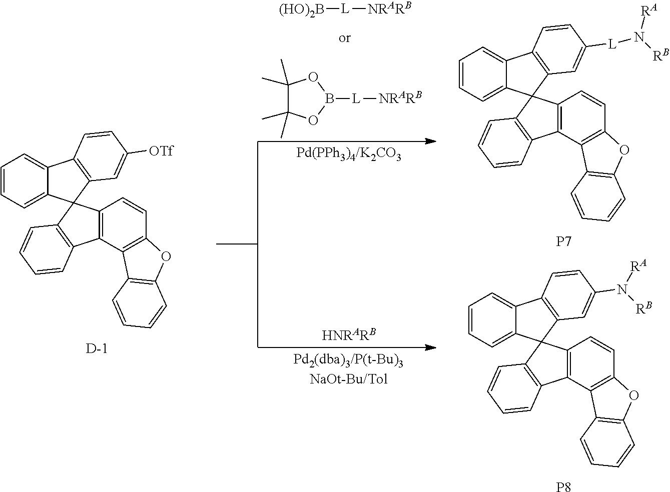

Preparation of Compound P7

A target compound P7 was obtained through a Suzuki coupling reaction using the compound D-1, a substituted boronic acid or a boronic ester compound, and a palladium catalyst.

Preparation of Compound P8

A target compound P8 was obtained through palladium-amination using the compound D-1, a secondary amine compound, and a palladium catalyst.

The structures of the substituted boronic acid, boronic ester, and secondary amine compound as the reactants and the structures and yields of the prepared compounds P7 and P8 are shown in Table 4.

TABLE 4

| Comp | | | |
|---|---|---|---|
| ound | | | |
| No. | Reactant | Product (P7, P8) | Yield |
| Com 26-1 | 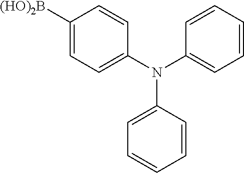 [SMILES: OB(O)c1ccc(N(c2ccccc2)c2ccccc2)cc1] | 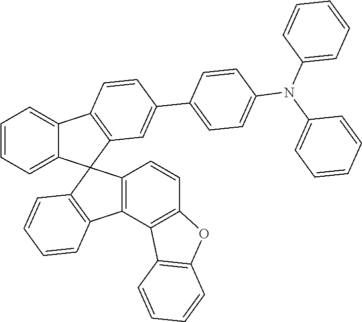 [SMILES: c1ccc(N(c2ccccc2)c2ccc(-c3ccc4c(c3)C3(c5ccccc5-4)c4ccccc4-c4c3ccc3oc5ccccc5c43)cc2)cc1] | 76% |
| Com 26-2 | 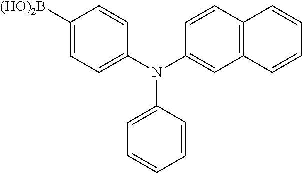 [SMILES: OB(O)c1ccc(N(c2ccccc2)c2ccc3ccccc3c2)cc1] | 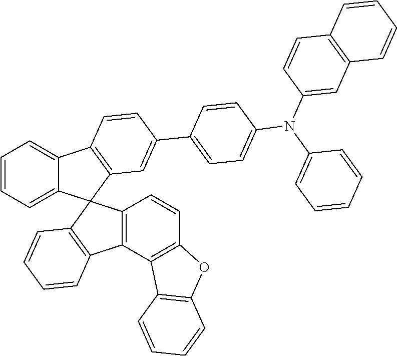 [SMILES: c1ccc(N(c2ccc(-c3ccc4c(c3)C3(c5ccccc5-4)c4ccccc4-c4c3ccc3oc5ccccc5c43)cc2)c2ccc3ccccc3c2)cc1] | 80% |
| Com 26-3 | 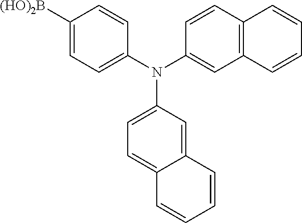 [SMILES: OB(O)c1ccc(N(c2ccc3ccccc3c2)c2ccc3ccccc3c2)cc1] | 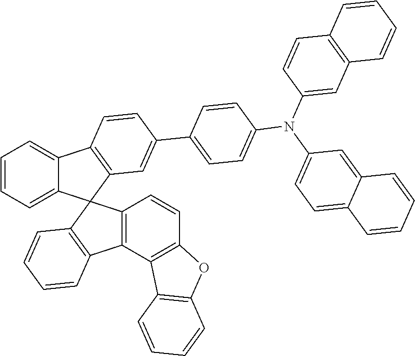 [SMILES: c1ccc2c(c1)-c1ccc(-c3ccc(N(c4ccc5ccccc5c4)c4ccc5ccccc5c4)cc3)cc1C21c2ccccc2-c2c1ccc1oc3ccccc3c21] | 71% |
| Com 26-4 | 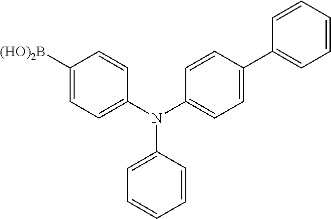 [SMILES: OB(O)c1ccc(N(c2ccccc2)c2ccc(-c3ccccc3)cc2)cc1] | 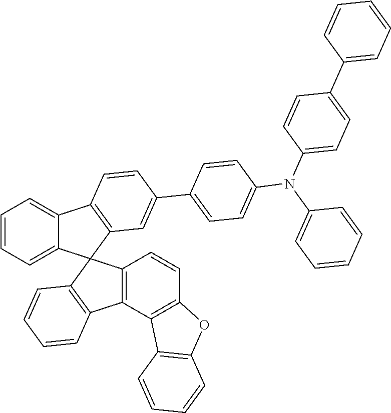 [SMILES: c1ccc(-c2ccc(N(c3ccccc3)c3ccc(-c4ccc5c(c4)C4(c6ccccc6-5)c5ccccc5-c5c4ccc4oc6ccccc6c54)cc3)cc2)cc1] | 75% |
| Com 26-5 | 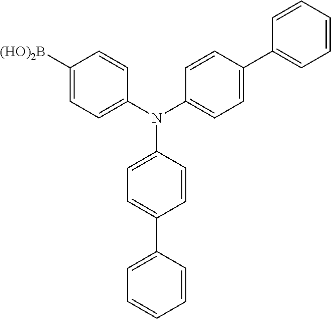 [SMILES: OB(O)c1ccc(N(c2ccc(-c3ccccc3)cc2)c2ccc(-c3ccccc3)cc2)cc1] | 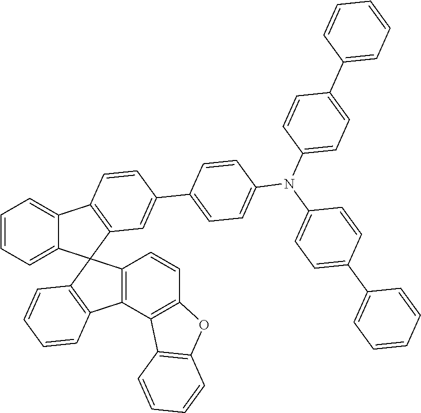 [SMILES: c1ccc(-c2ccc(N(c3ccc(-c4ccccc4)cc3)c3ccc(-c4ccc5c(c4)C4(c6ccccc6-5)c5ccccc5-c5c4ccc4oc6ccccc6c54)cc3)cc2)cc1] | 69% |
| Com 26-6 | 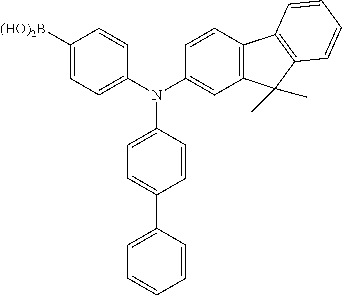 [SMILES: CC1(C)c2ccccc2-c2ccc(N(c3ccc(B(O)O)cc3)c3ccc(-c4ccccc4)cc3)cc21] | 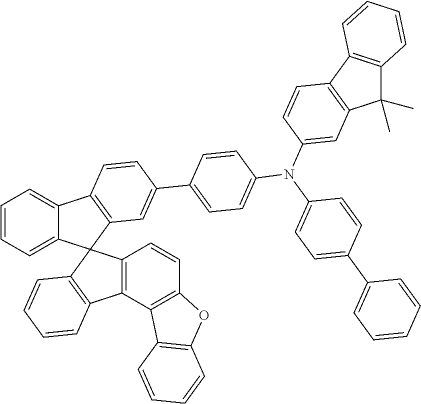 [SMILES: CC1(C)c2ccccc2-c2ccc(N(c3ccc(-c4ccccc4)cc3)c3ccc(-c4ccc5c(c4)C4(c6ccccc6-5)c5ccccc5-c5c4ccc4oc6ccccc6c54)cc3)cc21] | 75% |
| Com 26-7 | 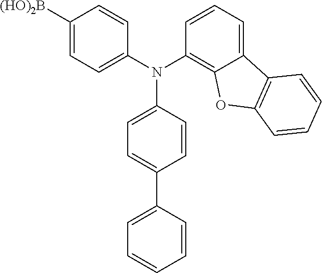 [SMILES: OB(O)c1ccc(N(c2ccc(-c3ccccc3)cc2)c2cccc3c2oc2ccccc23)cc1] | 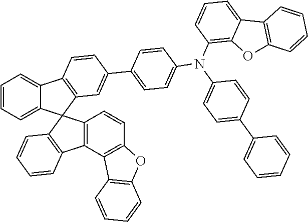 [SMILES: c1ccc(-c2ccc(N(c3ccc(-c4ccc5c(c4)C4(c6ccccc6-5)c5ccccc5-c5c4ccc4oc6ccccc6c54)cc3)c3cccc4c3oc3ccccc34)cc2)cc1] | 69% |
| Com 26-8 | 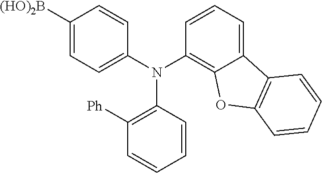 [SMILES: OB(O)c1ccc(N(c2ccccc2-c2ccccc2)c2cccc3c2oc2ccccc23)cc1] | 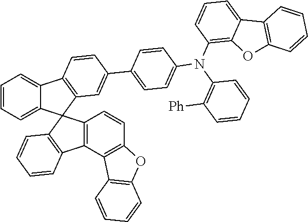 [SMILES: c1ccc(-c2ccccc2N(c2ccc(-c3ccc4c(c3)C3(c5ccccc5-4)c4ccccc4-c4c3ccc3oc5ccccc5c43)cc2)c2cccc3c2oc2ccccc23)cc1] | 65% |
| Com 26-9 | 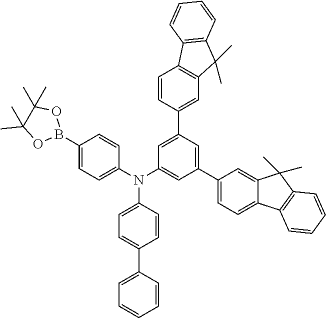 [SMILES: CC1(C)c2ccccc2-c2ccc(-c3cc(-c4ccc5c(c4)C(C)(C)c4ccccc4-5)cc(N(c4ccc(B5OC(C)(C)C(C)(C)O5)cc4)c4ccc(-c5ccccc5)cc4)c3)cc21] | 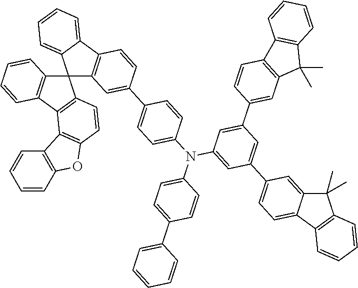 [SMILES: CC1(C)c2ccccc2-c2ccc(-c3cc(-c4ccc5c(c4)C(C)(C)c4ccccc4-5)cc(N(c4ccc(-c5ccccc5)cc4)c4ccc(-c5ccc6c(c5)C5(c7ccccc7-6)c6ccccc6-c6c5ccc5oc7ccccc7c65)cc4)c3)cc21] | 63% |
| Com 26-10 | 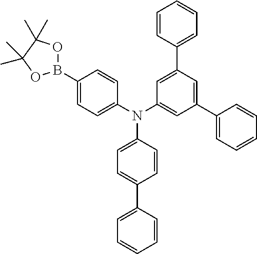 [SMILES: CC1(C)OB(c2ccc(N(c3ccc(-c4ccccc4)cc3)c3cc(-c4ccccc4)cc(-c4ccccc4)c3)cc2)OC1(C)C] | 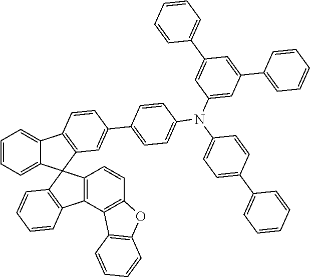 [SMILES: c1ccc(-c2ccc(N(c3ccc(-c4ccc5c(c4)C4(c6ccccc6-5)c5ccccc5-c5c4ccc4oc6ccccc6c54)cc3)c3cc(-c4ccccc4)cc(-c4ccccc4)c3)cc2)cc1] | 71% |
| Com 26-11 | 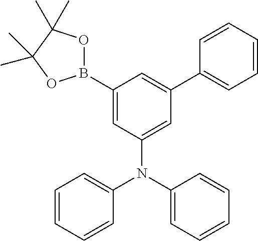 [SMILES: CC1(C)OB(c2cc(-c3ccccc3)cc(N(c3ccccc3)c3ccccc3)c2)OC1(C)C] | 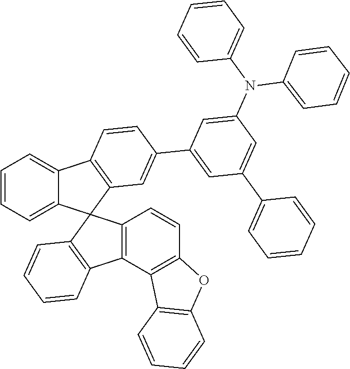 [SMILES: c1ccc(-c2cc(-c3ccc4c(c3)C3(c5ccccc5-4)c4ccccc4-c4c3ccc3oc5ccccc5c43)cc(N(c3ccccc3)c3ccccc3)c2)cc1] | 75% |
| Com 26-12 | 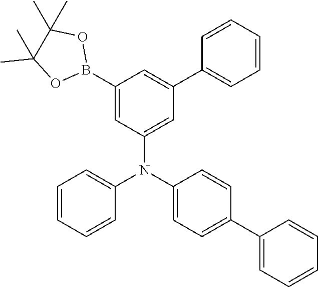 [SMILES: CC1(C)OB(c2cc(-c3ccccc3)cc(N(c3ccccc3)c3ccc(-c4ccccc4)cc3)c2)OC1(C)C] | 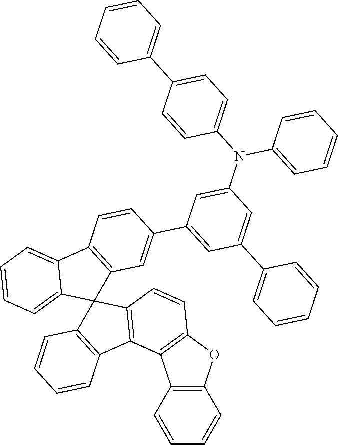 [SMILES: c1ccc(-c2ccc(N(c3ccccc3)c3cc(-c4ccccc4)cc(-c4ccc5c(c4)C4(c6ccccc6-5)c5ccccc5-c5c4ccc4oc6ccccc6c54)c3)cc2)cc1] | 69% |
| Com 26-13 | 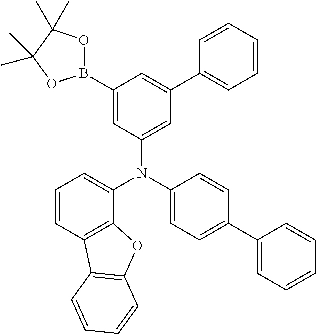 [SMILES: CC1(C)OB(c2cc(-c3ccccc3)cc(N(c3ccc(-c4ccccc4)cc3)c3cccc4c3oc3ccccc34)c2)OC1(C)C] | 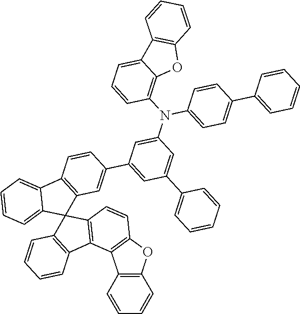 [SMILES: c1ccc(-c2ccc(N(c3cc(-c4ccccc4)cc(-c4ccc5c(c4)C4(c6ccccc6-5)c5ccccc5-c5c4ccc4oc6ccccc6c54)c3)c3cccc4c3oc3ccccc34)cc2)cc1] | 65% |
| Com 26-14 | 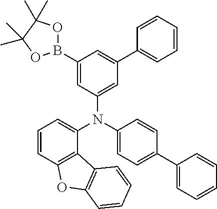 [SMILES: CC1(C)OB(c2cc(-c3ccccc3)cc(N(c3ccc(-c4ccccc4)cc3)c3cccc4oc5ccccc5c34)c2)OC1(C)C] | 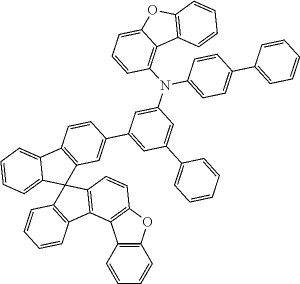 [SMILES: c1ccc(-c2ccc(N(c3cc(-c4ccccc4)cc(-c4ccc5c(c4)C4(c6ccccc6-5)c5ccccc5-c5c4ccc4oc6ccccc6c54)c3)c3cccc4oc5ccccc5c34)cc2)cc1] | 63% |
| Com 26-15 | 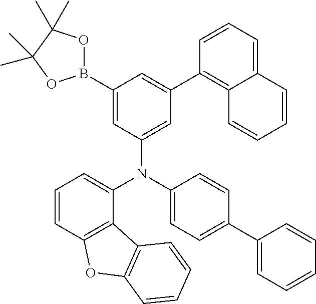 [SMILES: CC1(C)OB(c2cc(-c3cccc4ccccc34)cc(N(c3ccc(-c4ccccc4)cc3)c3cccc4oc5ccccc5c34)c2)OC1(C)C] | 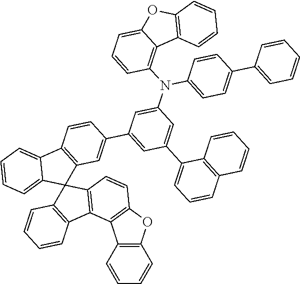 [SMILES: c1ccc(-c2ccc(N(c3cc(-c4ccc5c(c4)C4(c6ccccc6-5)c5ccccc5-c5c4ccc4oc6ccccc6c54)cc(-c4cccc5ccccc45)c3)c3cccc4oc5ccccc5c34)cc2)cc1] | 71% |
| Com 26-16 | 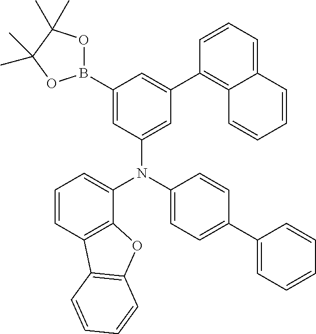 [SMILES: CC1(C)OB(c2cc(-c3cccc4ccccc34)cc(N(c3ccc(-c4ccccc4)cc3)c3cccc4c3oc3ccccc34)c2)OC1(C)C] | 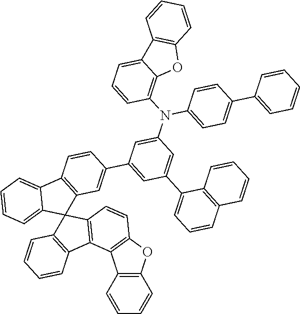 [SMILES: c1ccc(-c2ccc(N(c3cc(-c4ccc5c(c4)C4(c6ccccc6-5)c5ccccc5-c5c4ccc4oc6ccccc6c54)cc(-c4cccc5ccccc45)c3)c3cccc4c3oc3ccccc34)cc2)cc1] | 58% |
| Com 26-17 | 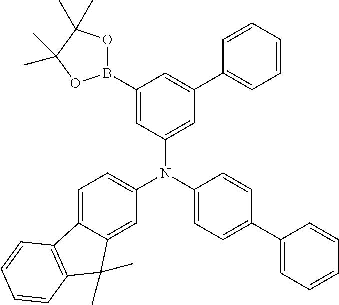 [SMILES: CC1(C)c2ccccc2-c2ccc(N(c3ccc(-c4ccccc4)cc3)c3cc(B4OC(C)(C)C(C)(C)O4)cc(-c4ccccc4)c3)cc21] | 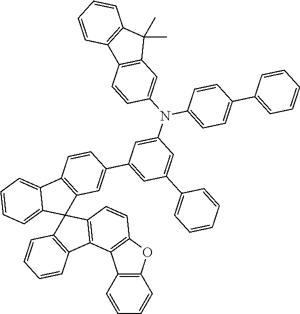 [SMILES: CC1(C)c2ccccc2-c2ccc(N(c3ccc(-c4ccccc4)cc3)c3cc(-c4ccccc4)cc(-c4ccc5c(c4)C4(c6ccccc6-5)c5ccccc5-c5c4ccc4oc6ccccc6c54)c3)cc21] | 61% |
| Com 26-18 | 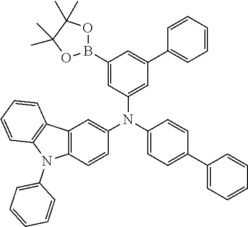 [SMILES: CC1(C)OB(c2cc(-c3ccccc3)cc(N(c3ccc(-c4ccccc4)cc3)c3ccc4c(c3)c3ccccc3n4-c3ccccc3)c2)OC1(C)C] | 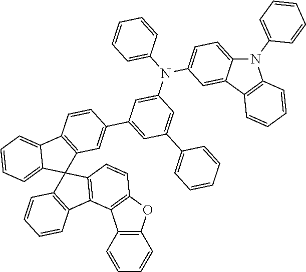 [SMILES: c1ccc(-c2cc(-c3ccc4c(c3)C3(c5ccccc5-4)c4ccccc4-c4c3ccc3oc5ccccc5c43)cc(N(c3ccccc3)c3ccc4c(c3)c3ccccc3n4-c3ccccc3)c2)cc1] | 69% |
| Com 26-19 | 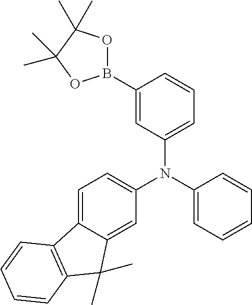 [SMILES: CC1(C)c2ccccc2-c2ccc(N(c3ccccc3)c3cccc(B4OC(C)(C)C(C)(C)O4)c3)cc21] | 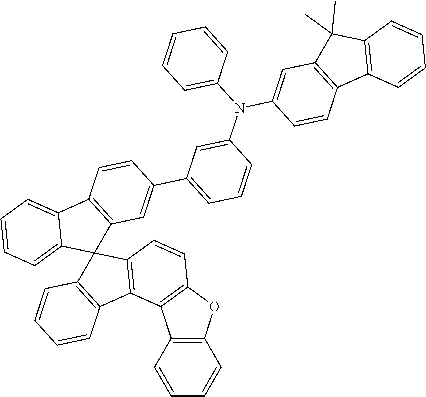 [SMILES: CC1(C)c2ccccc2-c2ccc(N(c3ccccc3)c3cccc(-c4ccc5c(c4)C4(c6ccccc6-5)c5ccccc5-c5c4ccc4oc6ccccc6c54)c3)cc21] | 74% |
| Com 26-20 | 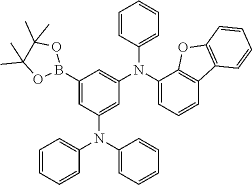 [SMILES: CC1(C)OB(c2cc(N(c3ccccc3)c3ccccc3)cc(N(c3ccccc3)c3cccc4c3oc3ccccc34)c2)OC1(C)C] | 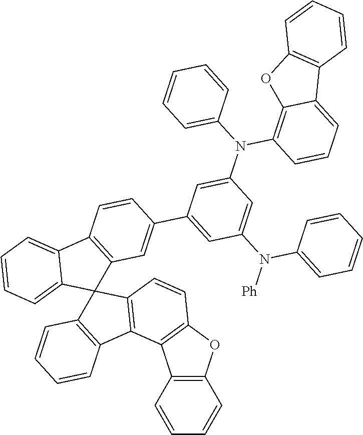 [SMILES: c1ccc(N(c2ccccc2)c2cc(-c3ccc4c(c3)C3(c5ccccc5-4)c4ccccc4-c4c3ccc3oc5ccccc5c43)cc(N(c3ccccc3)c3cccc4c3oc3ccccc34)c2)cc1] | 87% |
| Com 26-21 | 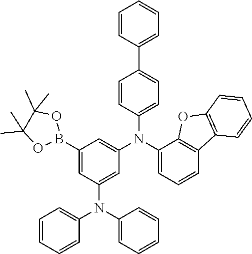 [SMILES: CC1(C)OB(c2cc(N(c3ccccc3)c3ccccc3)cc(N(c3ccc(-c4ccccc4)cc3)c3cccc4c3oc3ccccc34)c2)OC1(C)C] | 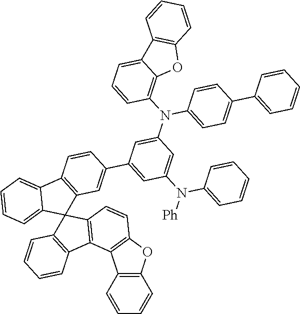 [SMILES: c1ccc(-c2ccc(N(c3cc(-c4ccc5c(c4)C4(c6ccccc6-5)c5ccccc5-c5c4ccc4oc6ccccc6c54)cc(N(c4ccccc4)c4ccccc4)c3)c3cccc4c3oc3ccccc34)cc2)cc1] | 81% |
| Com 26-22 | 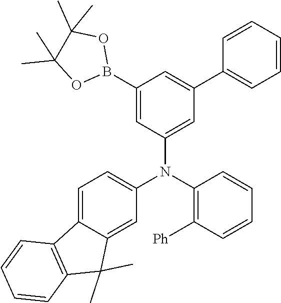 [SMILES: CC1(C)c2ccccc2-c2ccc(N(c3cc(B4OC(C)(C)C(C)(C)O4)cc(-c4ccccc4)c3)c3ccccc3-c3ccccc3)cc21] | 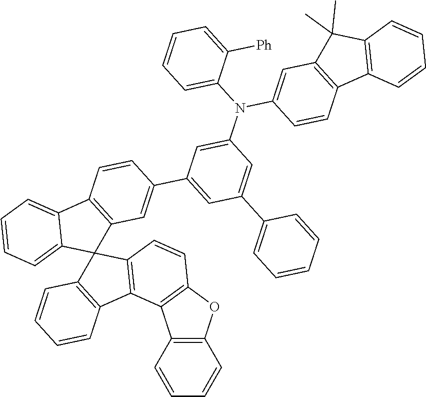 [SMILES: CC1(C)c2ccccc2-c2ccc(N(c3cc(-c4ccccc4)cc(-c4ccc5c(c4)C4(c6ccccc6-5)c5ccccc5-c5c4ccc4oc6ccccc6c54)c3)c3ccccc3-c3ccccc3)cc21] | 75% |
| Com 26-23 | 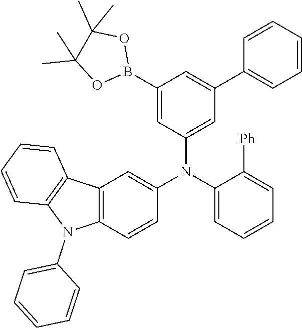 [SMILES: CC1(C)OB(c2cc(-c3ccccc3)cc(N(c3ccc4c(c3)c3ccccc3n4-c3ccccc3)c3ccccc3-c3ccccc3)c2)OC1(C)C] | 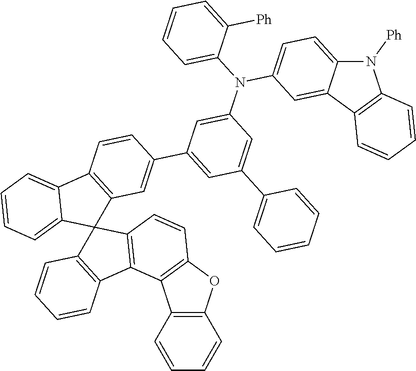 [SMILES: c1ccc(-c2cc(-c3ccc4c(c3)C3(c5ccccc5-4)c4ccccc4-c4c3ccc3oc5ccccc5c43)cc(N(c3ccc4c(c3)c3ccccc3n4-c3ccccc3)c3ccccc3-c3ccccc3)c2)cc1] | 80% |
| Com 26-24 | 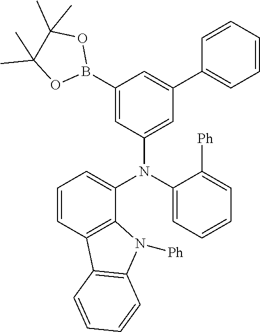 [SMILES: CC1(C)OB(c2cc(-c3ccccc3)cc(N(c3ccccc3-c3ccccc3)c3cccc4c5ccccc5n(-c5ccccc5)c34)c2)OC1(C)C] | 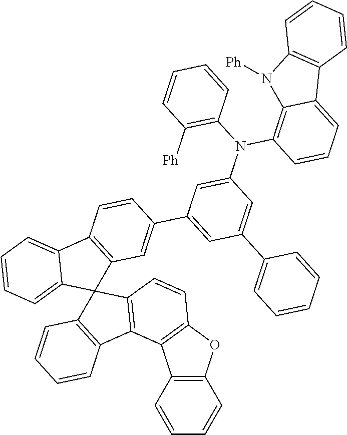 [SMILES: c1ccc(-c2cc(-c3ccc4c(c3)C3(c5ccccc5-4)c4ccccc4-c4c3ccc3oc5ccccc5c43)cc(N(c3ccccc3-c3ccccc3)c3cccc4c5ccccc5n(-c5ccccc5)c34)c2)cc1] | 62% |
| Com 26-25 | 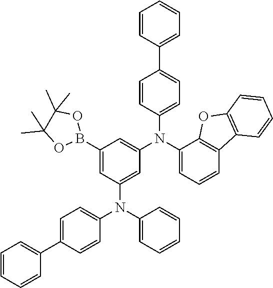 [SMILES: CC1(C)OB(c2cc(N(c3ccccc3)c3ccc(-c4ccccc4)cc3)cc(N(c3ccc(-c4ccccc4)cc3)c3cccc4c3oc3ccccc34)c2)OC1(C)C] | 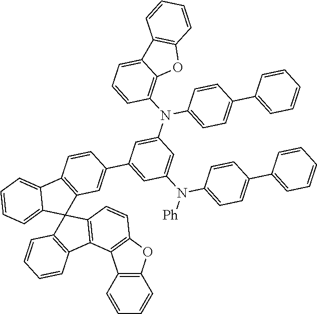 [SMILES: c1ccc(-c2ccc(N(c3ccccc3)c3cc(-c4ccc5c(c4)C4(c6ccccc6-5)c5ccccc5-c5c4ccc4oc6ccccc6c54)cc(N(c4ccc(-c5ccccc5)cc4)c4cccc5c4oc4ccccc45)c3)cc2)cc1] | 61% |
| Com 26-26 | 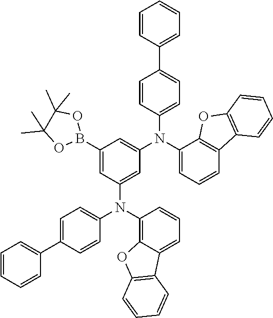 [SMILES: CC1(C)OB(c2cc(N(c3ccc(-c4ccccc4)cc3)c3cccc4c3oc3ccccc34)cc(N(c3ccc(-c4ccccc4)cc3)c3cccc4c3oc3ccccc34)c2)OC1(C)C] | 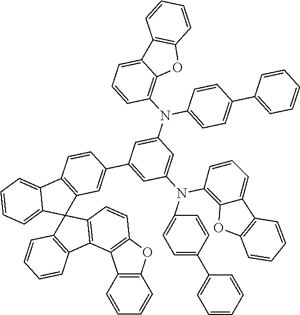 [SMILES: c1ccc(-c2ccc(N(c3cc(-c4ccc5c(c4)C4(c6ccccc6-5)c5ccccc5-c5c4ccc4oc6ccccc6c54)cc(N(c4ccc(-c5ccccc5)cc4)c4cccc5c4oc4ccccc45)c3)c3cccc4c3oc3ccccc34)cc2)cc1] | 64% |
| Com 26-27 | 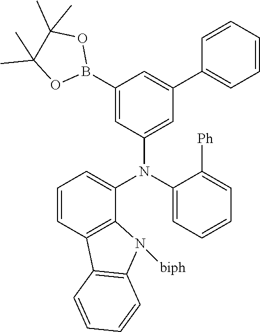 | 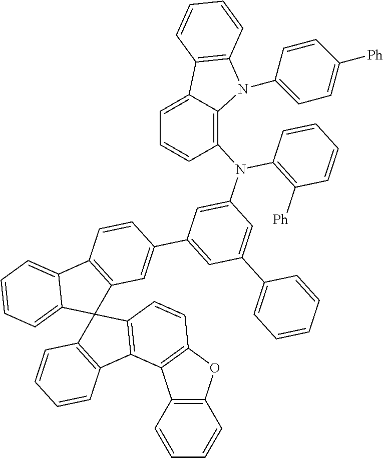 [SMILES: c1ccc(-c2ccc(-n3c4ccccc4c4cccc(N(c5cc(-c6ccccc6)cc(-c6ccc7c(c6)C6(c8ccccc8-7)c7ccccc7-c7c6ccc6oc8ccccc8c76)c5)c5ccccc5-c5ccccc5)c43)cc2)cc1] | 54% |
| Com 26-28 | 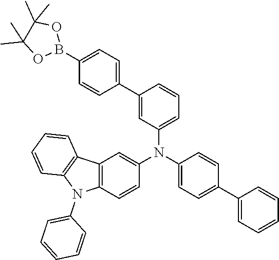 [SMILES: CC1(C)OB(c2ccc(-c3cccc(N(c4ccc(-c5ccccc5)cc4)c4ccc5c(c4)c4ccccc4n5-c4ccccc4)c3)cc2)OC1(C)C] | 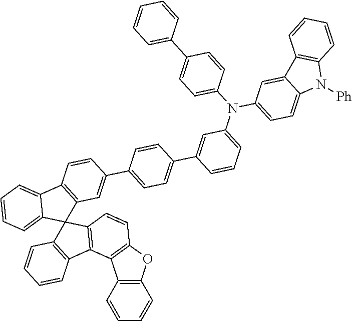 [SMILES: c1ccc(-c2ccc(N(c3cccc(-c4ccc(-c5ccc6c(c5)C5(c7ccccc7-6)c6ccccc6-c6c5ccc5oc7ccccc7c65)cc4)c3)c3ccc4c(c3)c3ccccc3n4-c3ccccc3)cc2)cc1] | 65% |
| Com 26-29 | 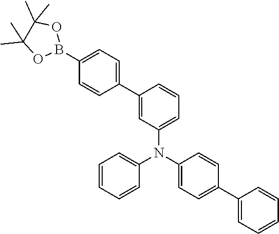 [SMILES: CC1(C)OB(c2ccc(-c3cccc(N(c4ccccc4)c4ccc(-c5ccccc5)cc4)c3)cc2)OC1(C)C] | 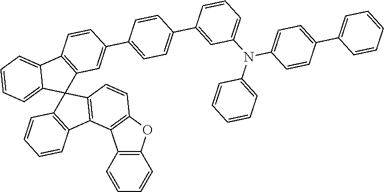 [SMILES: c1ccc(-c2ccc(N(c3ccccc3)c3cccc(-c4ccc(-c5ccc6c(c5)C5(c7ccccc7-6)c6ccccc6-c6c5ccc5oc7ccccc7c65)cc4)c3)cc2)cc1] | 78% |
| Com 26-30 | 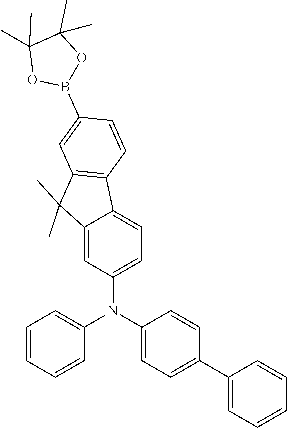 [SMILES: CC1(C)c2cc(B3OC(C)(C)C(C)(C)O3)ccc2-c2ccc(N(c3ccccc3)c3ccc(-c4ccccc4)cc3)cc21] | 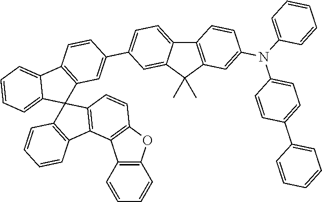 [SMILES: CC1(C)c2cc(-c3ccc4c(c3)C3(c5ccccc5-4)c4ccccc4-c4c3ccc3oc5ccccc5c43)ccc2-c2ccc(N(c3ccccc3)c3ccc(-c4ccccc4)cc3)cc21] | 80% |
| Com 26-31 | 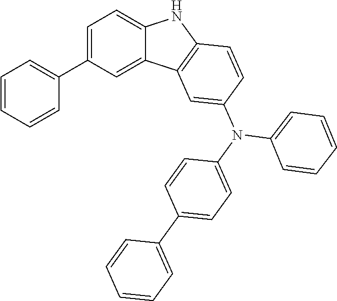 [SMILES: c1ccc(-c2ccc(N(c3ccccc3)c3ccc4[nH]c5ccc(-c6ccccc6)cc5c4c3)cc2)cc1] | 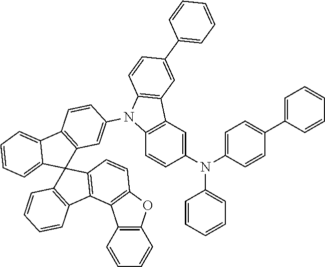 [SMILES: c1ccc(-c2ccc(N(c3ccccc3)c3ccc4c(c3)c3cc(-c5ccccc5)ccc3n4-c3ccc4c(c3)C3(c5ccccc5-4)c4ccccc4-c4c3ccc3oc5ccccc5c43)cc2)cc1] | 89% |
| Com 26-32 | 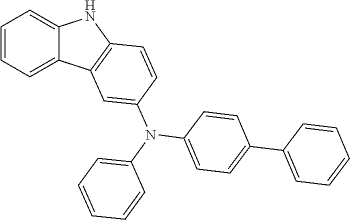 [SMILES: c1ccc(-c2ccc(N(c3ccccc3)c3ccc4[nH]c5ccccc5c4c3)cc2)cc1] | 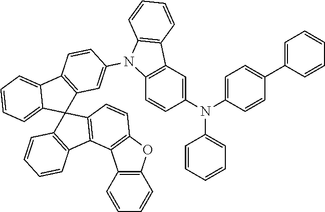 [SMILES: c1ccc(-c2ccc(N(c3ccccc3)c3ccc4c(c3)c3ccccc3n4-c3ccc4c(c3)C3(c5ccccc5-4)c4ccccc4-c4c3ccc3oc5ccccc5c43)cc2)cc1] | 91% |
| Com 26-33 | 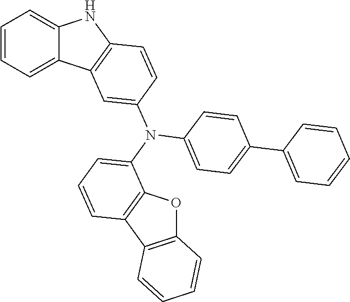 [SMILES: c1ccc(-c2ccc(N(c3ccc4[nH]c5ccccc5c4c3)c3cccc4c3oc3ccccc34)cc2)cc1] | 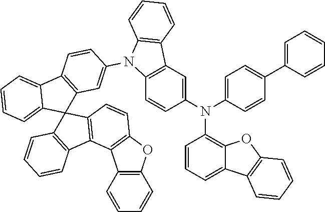 [SMILES: c1ccc(-c2ccc(N(c3ccc4c(c3)c3ccccc3n4-c3ccc4c(c3)C3(c5ccccc5-4)c4ccccc4-c4c3ccc3oc5ccccc5c43)c3cccc4c3oc3ccccc34)cc2)cc1] | 88% |
| Com 26-34 | 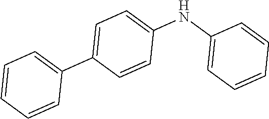 [SMILES: c1ccc(Nc2ccc(-c3ccccc3)cc2)cc1] | 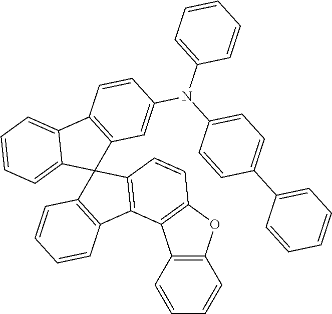 [SMILES: c1ccc(-c2ccc(N(c3ccccc3)c3ccc4c(c3)C3(c5ccccc5-4)c4ccccc4-c4c3ccc3oc5ccccc5c43)cc2)cc1] | 74% |
| Com 26-35 | 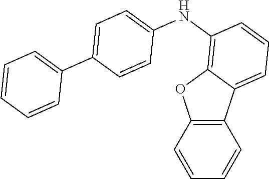 [SMILES: c1ccc(-c2ccc(Nc3cccc4c3oc3ccccc34)cc2)cc1] | 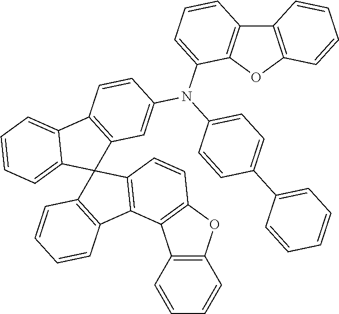 [SMILES: c1ccc(-c2ccc(N(c3ccc4c(c3)C3(c5ccccc5-4)c4ccccc4-c4c3ccc3oc5ccccc5c43)c3cccc4c3oc3ccccc34)cc2)cc1] | 92% |
| Com 26-36 | 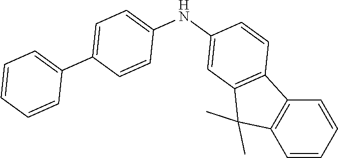 [SMILES: CC1(C)c2ccccc2-c2ccc(Nc3ccc(-c4ccccc4)cc3)cc21] | 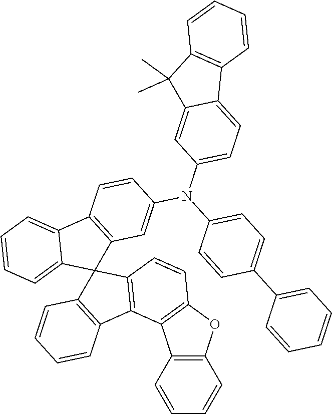 [SMILES: CC1(C)c2ccccc2-c2ccc(N(c3ccc(-c4ccccc4)cc3)c3ccc4c(c3)C3(c5ccccc5-4)c4ccccc4-c4c3ccc3oc5ccccc5c43)cc21] | 94% |
| Com 26-37 | 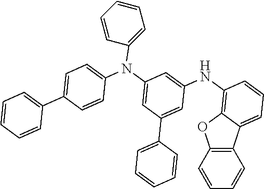 [SMILES: c1ccc(-c2ccc(N(c3ccccc3)c3cc(Nc4cccc5c4oc4ccccc45)cc(-c4ccccc4)c3)cc2)cc1] | 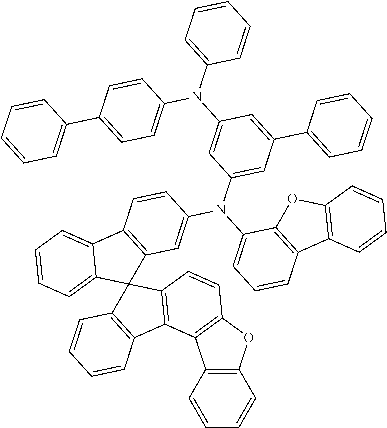 [SMILES: c1ccc(-c2ccc(N(c3ccccc3)c3cc(-c4ccccc4)cc(N(c4ccc5c(c4)C4(c6ccccc6-5)c5ccccc5-c5c4ccc4oc6ccccc6c54)c4cccc5c4oc4ccccc45)c3)cc2)cc1] | 93% |
| Com 26-38 | 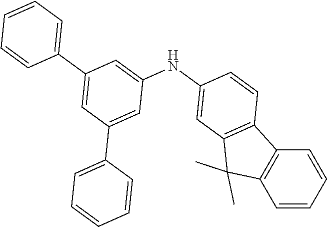 [SMILES: CC1(C)c2ccccc2-c2ccc(Nc3cc(-c4ccccc4)cc(-c4ccccc4)c3)cc21] | 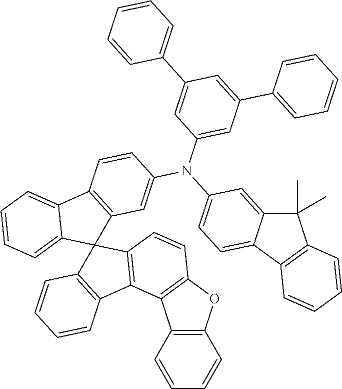 [SMILES: CC1(C)c2ccccc2-c2ccc(N(c3cc(-c4ccccc4)cc(-c4ccccc4)c3)c3ccc4c(c3)C3(c5ccccc5-4)c4ccccc4-c4c3ccc3oc5ccccc5c43)cc21] | 78% |
| Com 26-39 | 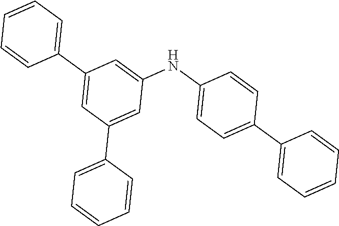 [SMILES: c1ccc(-c2ccc(Nc3cc(-c4ccccc4)cc(-c4ccccc4)c3)cc2)cc1] | 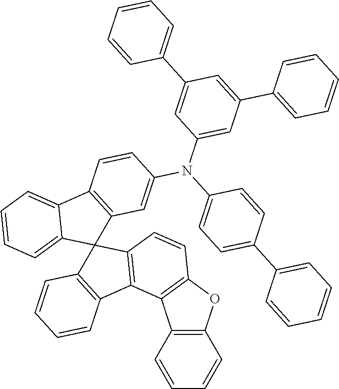 [SMILES: c1ccc(-c2ccc(N(c3cc(-c4ccccc4)cc(-c4ccccc4)c3)c3ccc4c(c3)C3(c5ccccc5-4)c4ccccc4-c4c3ccc3oc5ccccc5c43)cc2)cc1] | 89% |
| Com 26-40 | 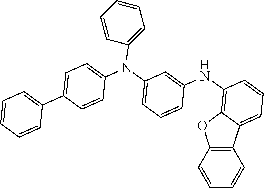 [SMILES: c1ccc(-c2ccc(N(c3ccccc3)c3cccc(Nc4cccc5c4oc4ccccc45)c3)cc2)cc1] | 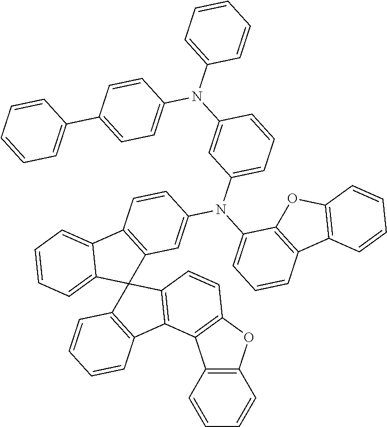 [SMILES: c1ccc(-c2ccc(N(c3ccccc3)c3cccc(N(c4ccc5c(c4)C4(c6ccccc6-5)c5ccccc5-c5c4ccc4oc6ccccc6c54)c4cccc5c4oc4ccccc45)c3)cc2)cc1] | 88% |
| Com 26-41 | 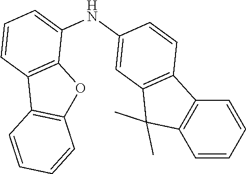 [SMILES: CC1(C)c2ccccc2-c2ccc(Nc3cccc4c3oc3ccccc34)cc21] | 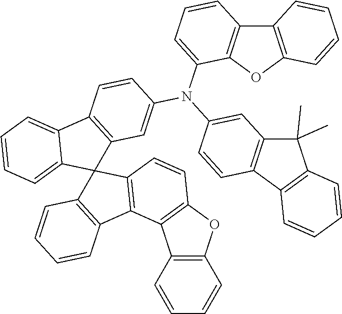 [SMILES: CC1(C)c2ccccc2-c2ccc(N(c3ccc4c(c3)C3(c5ccccc5-4)c4ccccc4-c4c3ccc3oc5ccccc5c43)c3cccc4c3oc3ccccc34)cc21] | 69% |
| Com 26-42 | 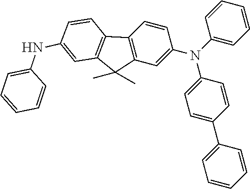 [SMILES: CC1(C)c2cc(Nc3ccccc3)ccc2-c2ccc(N(c3ccccc3)c3ccc(-c4ccccc4)cc3)cc21] | 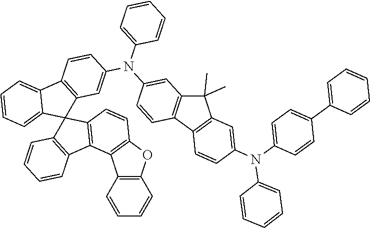 [SMILES: CC1(C)c2cc(N(c3ccccc3)c3ccc(-c4ccccc4)cc3)ccc2-c2ccc(N(c3ccccc3)c3ccc4c(c3)C3(c5ccccc5-4)c4ccccc4-c4c3ccc3oc5ccccc5c43)cc21] | 82% |
| Com 26-43 | 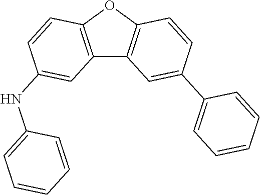 [SMILES: c1ccc(Nc2ccc3oc4ccc(-c5ccccc5)cc4c3c2)cc1] | 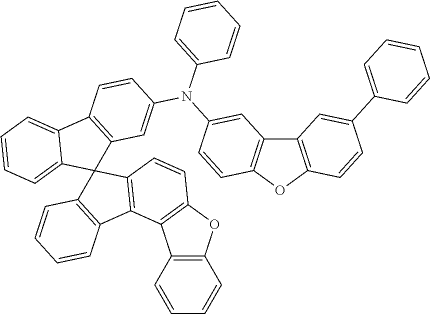 [SMILES: c1ccc(-c2ccc3oc4ccc(N(c5ccccc5)c5ccc6c(c5)C5(c7ccccc7-6)c6ccccc6-c6c5ccc5oc7ccccc7c65)cc4c3c2)cc1] | 73% |
| Com 26-44 | 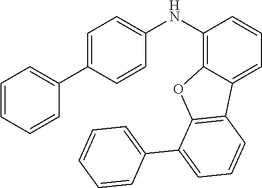 [SMILES: c1ccc(-c2ccc(Nc3cccc4c3oc3c(-c5ccccc5)cccc34)cc2)cc1] | 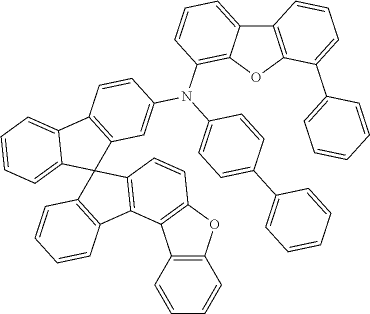 [SMILES: c1ccc(-c2ccc(N(c3ccc4c(c3)C3(c5ccccc5-4)c4ccccc4-c4c3ccc3oc5ccccc5c43)c3cccc4c3oc3c(-c5ccccc5)cccc34)cc2)cc1] | 83% |
| Com 26-45 | 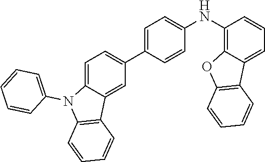 [SMILES: c1ccc(-n2c3ccccc3c3cc(-c4ccc(Nc5cccc6c5oc5ccccc56)cc4)ccc32)cc1] | 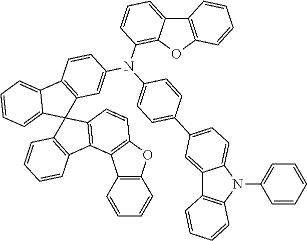 [SMILES: c1ccc(-n2c3ccccc3c3cc(-c4ccc(N(c5ccc6c(c5)C5(c7ccccc7-6)c6ccccc6-c6c5ccc5oc7ccccc7c65)c5cccc6c5oc5ccccc56)cc4)ccc32)cc1] | 86% |
| Com 26-46 | 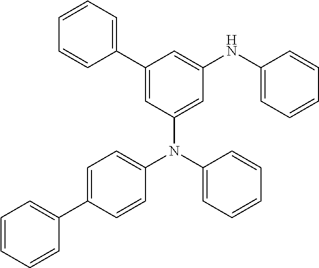 [SMILES: c1ccc(Nc2cc(-c3ccccc3)cc(N(c3ccccc3)c3ccc(-c4ccccc4)cc3)c2)cc1] | 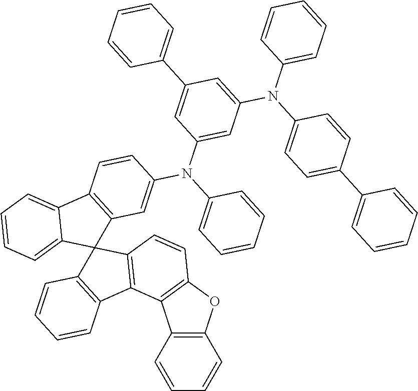 [SMILES: c1ccc(-c2ccc(N(c3ccccc3)c3cc(-c4ccccc4)cc(N(c4ccccc4)c4ccc5c(c4)C4(c6ccccc6-5)c5ccccc5-c5c4ccc4oc6ccccc6c54)c3)cc2)cc1] | 72% |
| Com 26-47 | 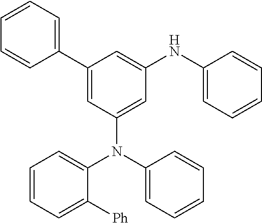 [SMILES: c1ccc(Nc2cc(-c3ccccc3)cc(N(c3ccccc3)c3ccccc3-c3ccccc3)c2)cc1] | 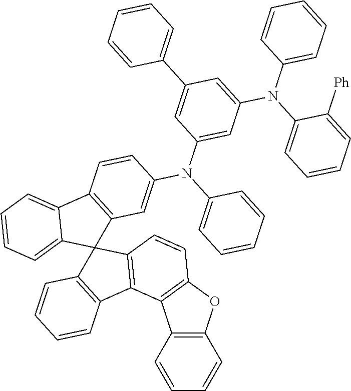 [SMILES: c1ccc(-c2cc(N(c3ccccc3)c3ccc4c(c3)C3(c5ccccc5-4)c4ccccc4-c4c3ccc3oc5ccccc5c43)cc(N(c3ccccc3)c3ccccc3-c3ccccc3)c2)cc1] | 88% |
| Com 26-48 | 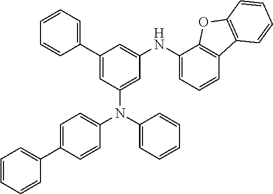 [SMILES: c1ccc(-c2ccc(N(c3ccccc3)c3cc(Nc4cccc5c4oc4ccccc45)cc(-c4ccccc4)c3)cc2)cc1] | 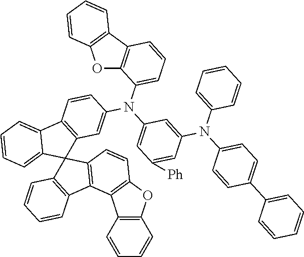 [SMILES: c1ccc(-c2ccc(N(c3ccccc3)c3cc(-c4ccccc4)cc(N(c4ccc5c(c4)C4(c6ccccc6-5)c5ccccc5-c5c4ccc4oc6ccccc6c54)c4cccc5c4oc4ccccc45)c3)cc2)cc1] | 69% |
| Com 26-49 | 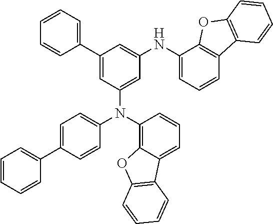 [SMILES: c1ccc(-c2ccc(N(c3cc(Nc4cccc5c4oc4ccccc45)cc(-c4ccccc4)c3)c3cccc4c3oc3ccccc34)cc2)cc1] | 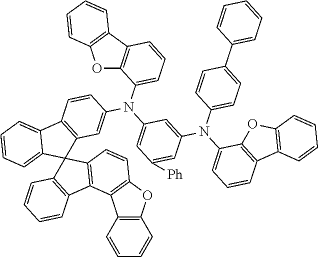 [SMILES: c1ccc(-c2ccc(N(c3cc(-c4ccccc4)cc(N(c4ccc5c(c4)C4(c6ccccc6-5)c5ccccc5-c5c4ccc4oc6ccccc6c54)c4cccc5c4oc4ccccc45)c3)c3cccc4c3oc3ccccc34)cc2)cc1] | 66% |
| Com 26-50 | 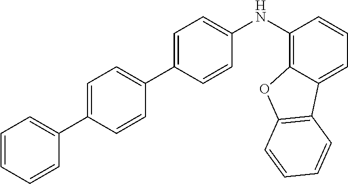 [SMILES: c1ccc(-c2ccc(-c3ccc(Nc4cccc5c4oc4ccccc45)cc3)cc2)cc1] | 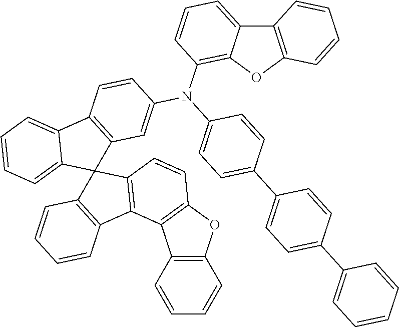 [SMILES: c1ccc(-c2ccc(-c3ccc(N(c4ccc5c(c4)C4(c6ccccc6-5)c5ccccc5-c5c4ccc4oc6ccccc6c54)c4cccc5c4oc4ccccc45)cc3)cc2)cc1] | 79% |

### [Preparation Example 5] Preparation of Compound E-1

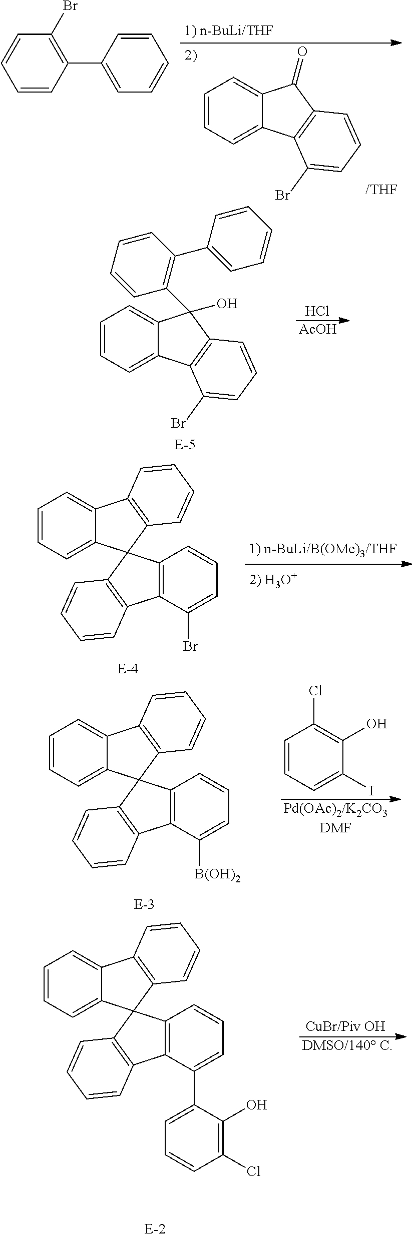

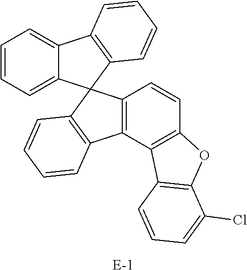

Preparation of Compound E-4

A compound E-4 was prepared in the same manner as that of Preparation Example 1, except that 2-bromo-1,1′-biphenyl (38.00 mmol) was used instead of 2-bromo-4′-methoxy-1,1′-biphenyl as a starting material.

Preparation of Compound E-3

10 g (23.51 mmol) of the compound E-4 was dissolved in 100 mL of THF, the temperature was lowered to −78° C., 18 mL (28.21 mmol) of n-butyllithium (1.6 M solution in hexane) was slowly added dropwise thereto, and stirring was performed at the same temperature for 30 minutes. While stirring was continued, the temperature was slowly raised to 0° C., and stirring was performed while slowly adding 3.2 mL (28.21 mmol) of trimethyl borate dropwise at 0° C. Stirring was performed at the same temperature for 2 hours, and stirring was performed for 18 hours while raising the temperature to room temperature. After the reaction was completed, the reaction was stopped with a 1 N HCl aqueous solution, extraction was performed using MC and H₂O, the solvent was removed, and recrystallization was performed with a small amount of THF and Hex, thereby obtaining 5.96 g (65%) of a target compound E-3.

Preparation of Compound E-2

10 g (25.62 mmol) of the compound E-3 and 7.82 g (30.75 mmol) of 2-chloro-6-iodophenol were dissolved in 110 mL of DMF, 0.86 g (3.84 mmol) of palladium acetate(II) and 10.62 g (76.86 mmol) of K₂CO₃ were added thereto under stirring, and stirring was performed under reflux for 24 hours. A sodium bicarbonate (NaHCO₃) aqueous solution was added to stop the reaction, extraction was performed using MC, the solvent was removed, and recrystallization purification was performed with DMF and Hex, thereby obtaining 6.13 g (54%) of a target compound E-2.

Preparation of Compound E-1

10 g (22.58 mmol) of the compound E-2 was dissolved in 120 mL of DMSO, 3.24 g (22.58 mmol) of copper(I) bromide (CuBr) and 5.07 mL (45.16 mmol) of pivalic acid were added, and stirring was performed under reflux at 140° C. for 24 hours. After the reaction was stopped, the reaction solution was cooled to room temperature, extraction was performed with a sodium bicarbonate (NaHCO₃) aqueous solution and MC, the solvent was removed, and the produced solid was washed with Hex and EA. Recrystallization was performed using DMF and Hex, thereby obtaining 6.67 g (67%) of a target compound E-1.

### [Example 5] Preparation of Compounds P9 and P10

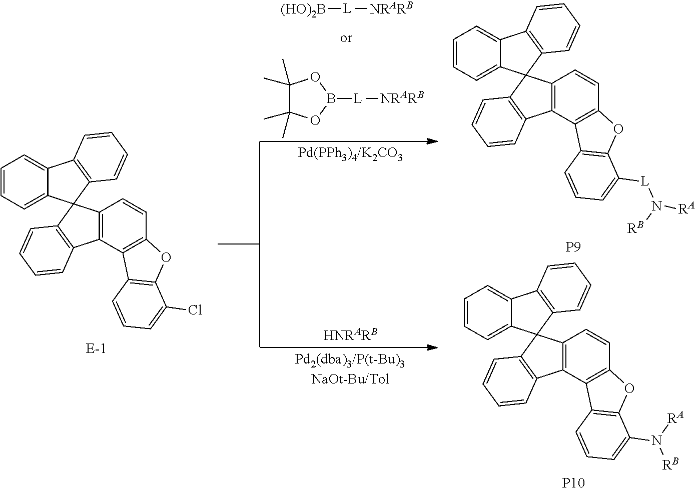

Preparation of Compound P9

A target compound P9 was obtained through a Suzuki coupling reaction using the compound E-1, a substituted boronic acid or a boronic ester compound, and a palladium catalyst.

Preparation of Compound P10

A target compound P10 was obtained through palladium-amination using the compound E-1, a secondary amine compound, and a palladium catalyst.

The structures of the substituted boronic acid, boronic ester, and secondary amine compound as the reactants and the structures and yields of the prepared compounds P9 and P10 are shown in Table 5.

TABLE 5

| Compound | | | |
|---|---|---|---|
| No. | Reactant | Product (P9, P10) | Yield |
| Com 27-1 | 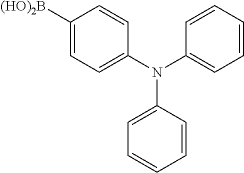 [SMILES: OB(O)c1ccc(N(c2ccccc2)c2ccccc2)cc1] | 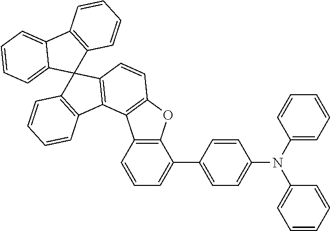 [SMILES: c1ccc(N(c2ccccc2)c2ccc(-c3cccc4c3oc3ccc5c(c34)-c3ccccc3C53c4ccccc4-c4ccccc43)cc2)cc1] | 81% |
| Com 27-2 | 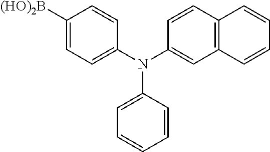 [SMILES: OB(O)c1ccc(N(c2ccccc2)c2ccc3ccccc3c2)cc1] | 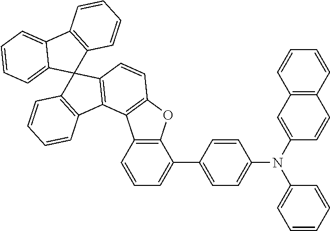 [SMILES: c1ccc(N(c2ccc(-c3cccc4c3oc3ccc5c(c34)-c3ccccc3C53c4ccccc4-c4ccccc43)cc2)c2ccc3ccccc3c2)cc1] | 78% |
| Com 27-3 | 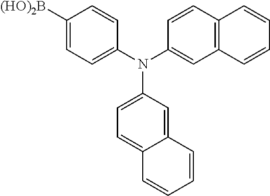 [SMILES: OB(O)c1ccc(N(c2ccc3ccccc3c2)c2ccc3ccccc3c2)cc1] | 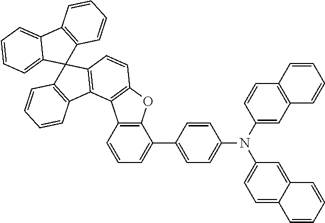 [SMILES: c1ccc2c(c1)-c1ccccc1C21c2ccccc2-c2c1ccc1oc3c(-c4ccc(N(c5ccc6ccccc6c5)c5ccc6ccccc6c5)cc4)cccc3c21] | 81% |
| Com 27-4 | 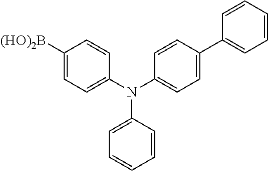 [SMILES: OB(O)c1ccc(N(c2ccccc2)c2ccc(-c3ccccc3)cc2)cc1] | 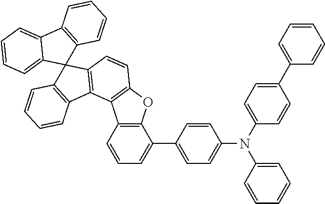 [SMILES: c1ccc(-c2ccc(N(c3ccccc3)c3ccc(-c4cccc5c4oc4ccc6c(c45)-c4ccccc4C64c5ccccc5-c5ccccc54)cc3)cc2)cc1] | 89% |
| Com 27-5 | 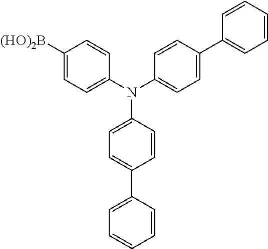 [SMILES: OB(O)c1ccc(N(c2ccc(-c3ccccc3)cc2)c2ccc(-c3ccccc3)cc2)cc1] | 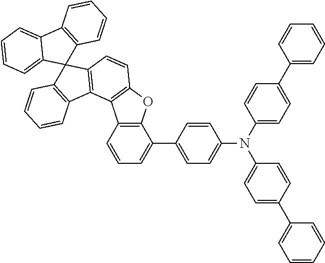 [SMILES: c1ccc(-c2ccc(N(c3ccc(-c4ccccc4)cc3)c3ccc(-c4cccc5c4oc4ccc6c(c45)-c4ccccc4C64c5ccccc5-c5ccccc54)cc3)cc2)cc1] | 74% |
| Com 27-6 | 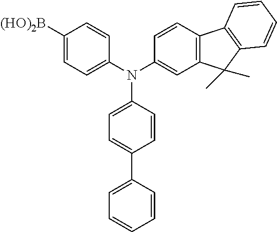 [SMILES: CC1(C)c2ccccc2-c2ccc(N(c3ccc(B(O)O)cc3)c3ccc(-c4ccccc4)cc3)cc21] | 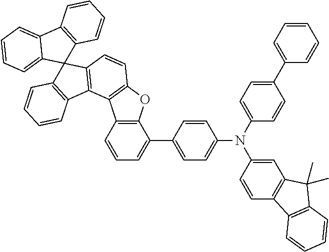 [SMILES: CC1(C)c2ccccc2-c2ccc(N(c3ccc(-c4ccccc4)cc3)c3ccc(-c4cccc5c4oc4ccc6c(c45)-c4ccccc4C64c5ccccc5-c5ccccc54)cc3)cc21] | 76% |
| Com 27-7 | 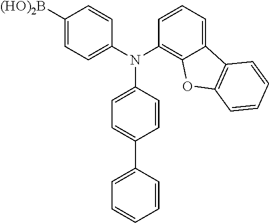 [SMILES: OB(O)c1ccc(N(c2ccc(-c3ccccc3)cc2)c2cccc3c2oc2ccccc23)cc1] | 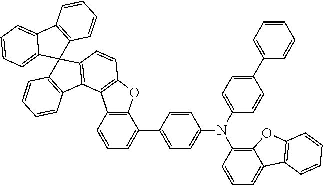 [SMILES: c1ccc(-c2ccc(N(c3ccc(-c4cccc5c4oc4ccc6c(c45)-c4ccccc4C64c5ccccc5-c5ccccc54)cc3)c3cccc4c3oc3ccccc34)cc2)cc1] | 75% |
| Com 27-8 | 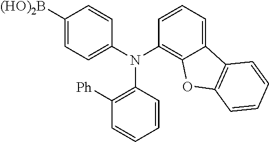 [SMILES: OB(O)c1ccc(N(c2ccccc2-c2ccccc2)c2cccc3c2oc2ccccc23)cc1] | 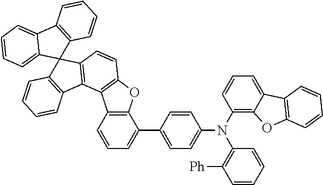 [SMILES: c1ccc(-c2ccccc2N(c2ccc(-c3cccc4c3oc3ccc5c(c34)-c3ccccc3C53c4ccccc4-c4ccccc43)cc2)c2cccc3c2oc2ccccc23)cc1] | 74% |
| Com 27-9 | 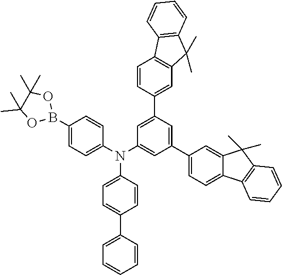 [SMILES: CC1(C)c2ccccc2-c2ccc(-c3cc(-c4ccc5c(c4)C(C)(C)c4ccccc4-5)cc(N(c4ccc(B5OC(C)(C)C(C)(C)O5)cc4)c4ccc(-c5ccccc5)cc4)c3)cc21] | 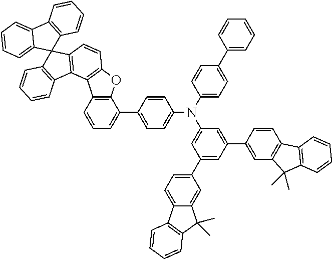 [SMILES: CC1(C)c2ccccc2-c2ccc(-c3cc(-c4ccc5c(c4)C(C)(C)c4ccccc4-5)cc(N(c4ccc(-c5ccccc5)cc4)c4ccc(-c5cccc6c5oc5ccc7c(c56)-c5ccccc5C75c6ccccc6-c6ccccc65)cc4)c3)cc21] | 58% |
| Com 27-10 | 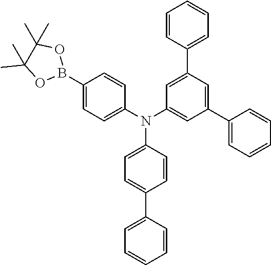 [SMILES: CC1(C)OB(c2ccc(N(c3ccc(-c4ccccc4)cc3)c3cc(-c4ccccc4)cc(-c4ccccc4)c3)cc2)OC1(C)C] | 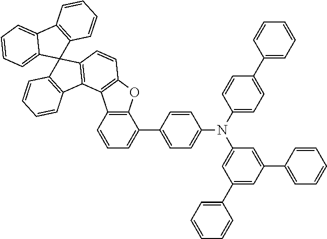 [SMILES: c1ccc(-c2ccc(N(c3ccc(-c4cccc5c4oc4ccc6c(c45)-c4ccccc4C64c5ccccc5-c5ccccc54)cc3)c3cc(-c4ccccc4)cc(-c4ccccc4)c3)cc2)cc1] | 72% |
| Com 27-11 | 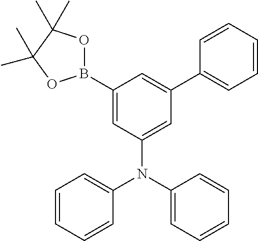 [SMILES: CC1(C)OB(c2cc(-c3ccccc3)cc(N(c3ccccc3)c3ccccc3)c2)OC1(C)C] | 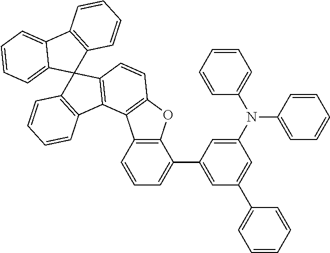 [SMILES: c1ccc(-c2cc(-c3cccc4c3oc3ccc5c(c34)-c3ccccc3C53c4ccccc4-c4ccccc43)cc(N(c3ccccc3)c3ccccc3)c2)cc1] | 92% |
| Com 27-12 | 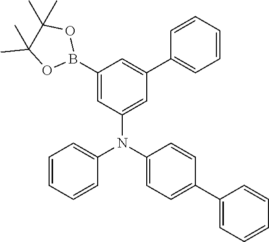 [SMILES: CC1(C)OB(c2cc(-c3ccccc3)cc(N(c3ccccc3)c3ccc(-c4ccccc4)cc3)c2)OC1(C)C] | 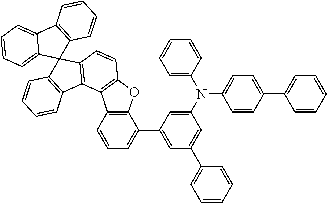 [SMILES: c1ccc(-c2ccc(N(c3ccccc3)c3cc(-c4ccccc4)cc(-c4cccc5c4oc4ccc6c(c45)-c4ccccc4C64c5ccccc5-c5ccccc54)c3)cc2)cc1] | 94% |
| Com 27-13 | 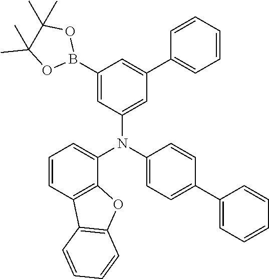 [SMILES: CC1(C)OB(c2cc(-c3ccccc3)cc(N(c3ccc(-c4ccccc4)cc3)c3cccc4c3oc3ccccc34)c2)OC1(C)C] | 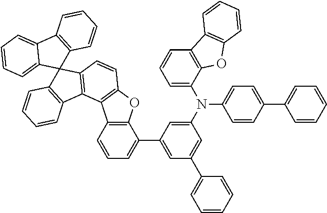 [SMILES: c1ccc(-c2ccc(N(c3cc(-c4ccccc4)cc(-c4cccc5c4oc4ccc6c(c45)-c4ccccc4C64c5ccccc5-c5ccccc54)c3)c3cccc4c3oc3ccccc34)cc2)cc1] | 84% |
| Com 27-14 | 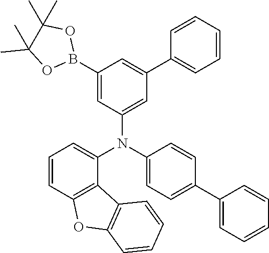 [SMILES: CC1(C)OB(c2cc(-c3ccccc3)cc(N(c3ccc(-c4ccccc4)cc3)c3cccc4oc5ccccc5c34)c2)OC1(C)C] | 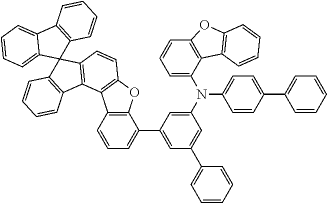 [SMILES: c1ccc(-c2ccc(N(c3cc(-c4ccccc4)cc(-c4cccc5c4oc4ccc6c(c45)-c4ccccc4C64c5ccccc5-c5ccccc54)c3)c3cccc4oc5ccccc5c34)cc2)cc1] | 88% |
| Com 27-15 | 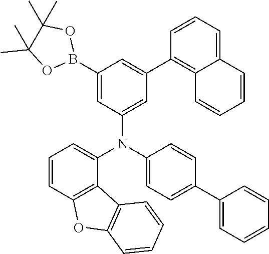 [SMILES: CC1(C)OB(c2cc(-c3cccc4ccccc34)cc(N(c3ccc(-c4ccccc4)cc3)c3cccc4oc5ccccc5c34)c2)OC1(C)C] | 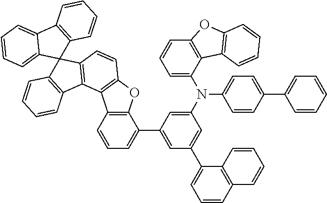 [SMILES: c1ccc(-c2ccc(N(c3cc(-c4cccc5ccccc45)cc(-c4cccc5c4oc4ccc6c(c45)-c4ccccc4C64c5ccccc5-c5ccccc54)c3)c3cccc4oc5ccccc5c34)cc2)cc1] | 78% |
| Com 27-16 | 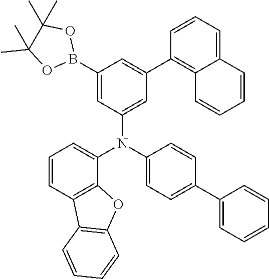 [SMILES: CC1(C)OB(c2cc(-c3cccc4ccccc34)cc(N(c3ccc(-c4ccccc4)cc3)c3cccc4c3oc3ccccc34)c2)OC1(C)C] | 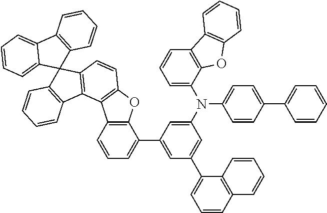 [SMILES: c1ccc(-c2ccc(N(c3cc(-c4cccc5ccccc45)cc(-c4cccc5c4oc4ccc6c(c45)-c4ccccc4C64c5ccccc5-c5ccccc54)c3)c3cccc4c3oc3ccccc34)cc2)cc1] | 71% |
| Com 27-17 | 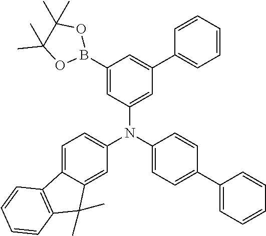 [SMILES: CC1(C)c2ccccc2-c2ccc(N(c3ccc(-c4ccccc4)cc3)c3cc(B4OC(C)(C)C(C)(C)O4)cc(-c4ccccc4)c3)cc21] | 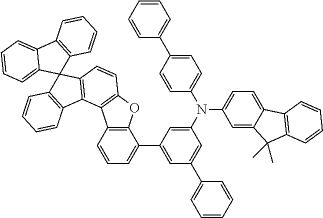 [SMILES: CC1(C)c2ccccc2-c2ccc(N(c3ccc(-c4ccccc4)cc3)c3cc(-c4ccccc4)cc(-c4cccc5c4oc4ccc6c(c45)-c4ccccc4C64c5ccccc5-c5ccccc54)c3)cc21] | 78% |
| Com 27-18 | 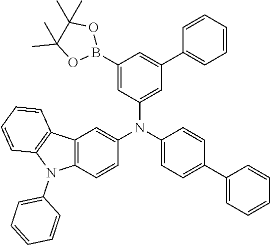 [SMILES: CC1(C)OB(c2cc(-c3ccccc3)cc(N(c3ccc(-c4ccccc4)cc3)c3ccc4c(c3)c3ccccc3n4-c3ccccc3)c2)OC1(C)C] | 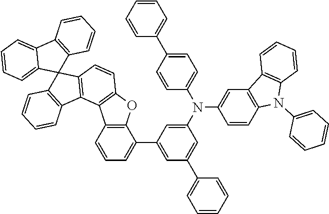 [SMILES: c1ccc(-c2ccc(N(c3cc(-c4ccccc4)cc(-c4cccc5c4oc4ccc6c(c45)-c4ccccc4C64c5ccccc5-c5ccccc54)c3)c3ccc4c(c3)c3ccccc3n4-c3ccccc3)cc2)cc1] | 74% |
| Com 27-19 | 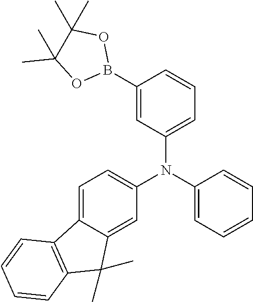 [SMILES: CC1(C)c2ccccc2-c2ccc(N(c3ccccc3)c3cccc(B4OC(C)(C)C(C)(C)O4)c3)cc21] | 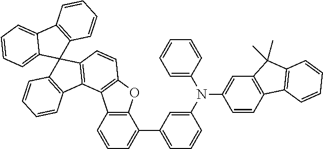 [SMILES: CC1(C)c2ccccc2-c2ccc(N(c3ccccc3)c3cccc(-c4cccc5c4oc4ccc6c(c45)-c4ccccc4C64c5ccccc5-c5ccccc54)c3)cc21] | 65% |
| Com 27-20 | 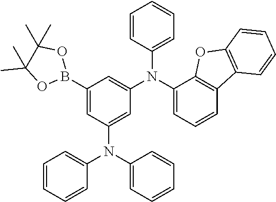 [SMILES: CC1(C)OB(c2cc(N(c3ccccc3)c3ccccc3)cc(N(c3ccccc3)c3cccc4c3oc3ccccc34)c2)OC1(C)C] | 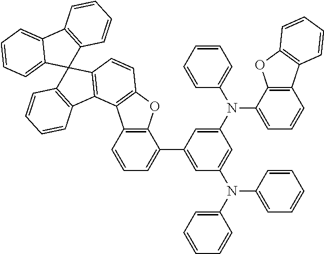 [SMILES: c1ccc(N(c2ccccc2)c2cc(-c3cccc4c3oc3ccc5c(c34)-c3ccccc3C53c4ccccc4-c4ccccc43)cc(N(c3ccccc3)c3cccc4c3oc3ccccc34)c2)cc1] | 74% |
| Com 27-21 | 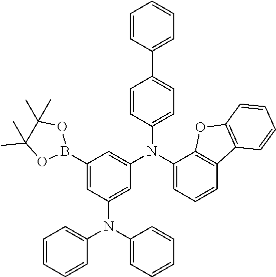 [SMILES: CC1(C)OB(c2cc(N(c3ccccc3)c3ccccc3)cc(N(c3ccc(-c4ccccc4)cc3)c3cccc4c3oc3ccccc34)c2)OC1(C)C] | 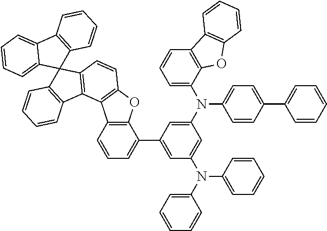 [SMILES: c1ccc(-c2ccc(N(c3cc(-c4cccc5c4oc4ccc6c(c45)-c4ccccc4C64c5ccccc5-c5ccccc54)cc(N(c4ccccc4)c4ccccc4)c3)c3cccc4c3oc3ccccc34)cc2)cc1] | 75% |
| Com 27-22 | 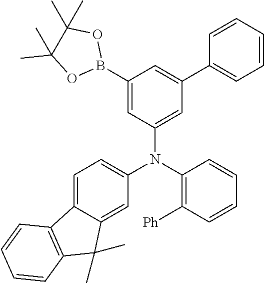 [SMILES: CC1(C)c2ccccc2-c2ccc(N(c3cc(B4OC(C)(C)C(C)(C)O4)cc(-c4ccccc4)c3)c3ccccc3-c3ccccc3)cc21] | 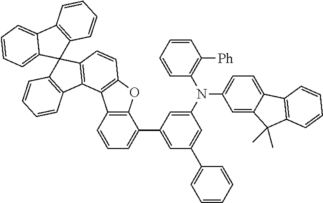 [SMILES: CC1(C)c2ccccc2-c2ccc(N(c3cc(-c4ccccc4)cc(-c4cccc5c4oc4ccc6c(c45)-c4ccccc4C64c5ccccc5-c5ccccc54)c3)c3ccccc3-c3ccccc3)cc21] | 72% |
| Com 27-23 | 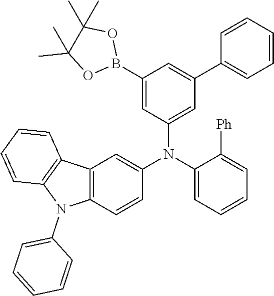 [SMILES: CC1(C)OB(c2cc(-c3ccccc3)cc(N(c3ccc4c(c3)c3ccccc3n4-c3ccccc3)c3ccccc3-c3ccccc3)c2)OC1(C)C] | 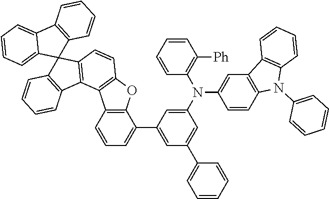 [SMILES: c1ccc(-c2cc(-c3cccc4c3oc3ccc5c(c34)-c3ccccc3C53c4ccccc4-c4ccccc43)cc(N(c3ccc4c(c3)c3ccccc3n4-c3ccccc3)c3ccccc3-c3ccccc3)c2)cc1] | 77% |
| Com 27-24 | 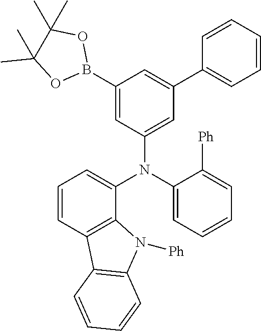 [SMILES: CC1(C)OB(c2cc(-c3ccccc3)cc(N(c3ccccc3-c3ccccc3)c3cccc4c5ccccc5n(-c5ccccc5)c34)c2)OC1(C)C] | 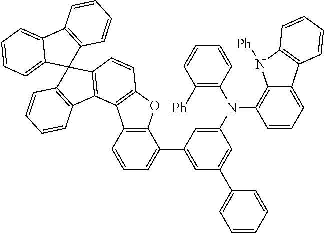 [SMILES: c1ccc(-c2cc(-c3cccc4c3oc3ccc5c(c34)-c3ccccc3C53c4ccccc4-c4ccccc43)cc(N(c3ccccc3-c3ccccc3)c3cccc4c5ccccc5n(-c5ccccc5)c34)c2)cc1] | 70% |
| Com 27-25 | 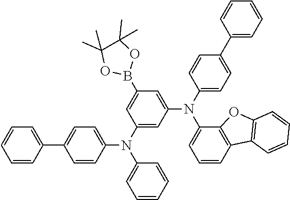 [SMILES: CC1(C)OB(c2cc(N(c3ccccc3)c3ccc(-c4ccccc4)cc3)cc(N(c3ccc(-c4ccccc4)cc3)c3cccc4c3oc3ccccc34)c2)OC1(C)C] | 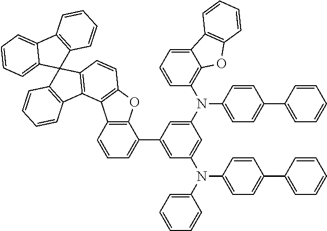 [SMILES: c1ccc(-c2ccc(N(c3ccccc3)c3cc(-c4cccc5c4oc4ccc6c(c45)-c4ccccc4C64c5ccccc5-c5ccccc54)cc(N(c4ccc(-c5ccccc5)cc4)c4cccc5c4oc4ccccc45)c3)cc2)cc1] | 72% |
| Com 27-26 | 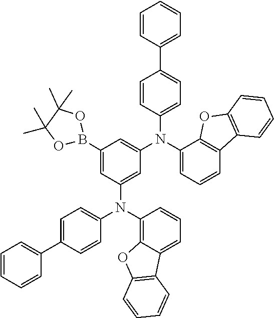 [SMILES: CC1(C)OB(c2cc(N(c3ccc(-c4ccccc4)cc3)c3cccc4c3oc3ccccc34)cc(N(c3ccc(-c4ccccc4)cc3)c3cccc4c3oc3ccccc34)c2)OC1(C)C] | 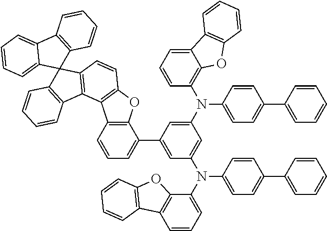 [SMILES: c1ccc(-c2ccc(N(c3cc(-c4cccc5c4oc4ccc6c(c45)-c4ccccc4C64c5ccccc5-c5ccccc54)cc(N(c4ccc(-c5ccccc5)cc4)c4cccc5c4oc4ccccc45)c3)c3cccc4c3oc3ccccc34)cc2)cc1] | 63% |
| Com 27-27 | 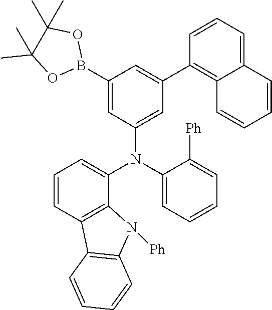 [SMILES: CC1(C)OB(c2cc(-c3cccc4ccccc34)cc(N(c3ccccc3-c3ccccc3)c3cccc4c5ccccc5n(-c5ccccc5)c34)c2)OC1(C)C] | 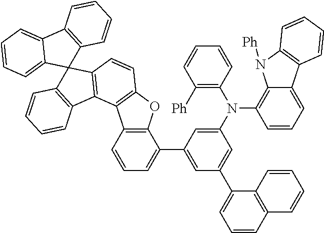 [SMILES: c1ccc(-c2ccccc2N(c2cc(-c3cccc4ccccc34)cc(-c3cccc4c3oc3ccc5c(c34)-c3ccccc3C53c4ccccc4-c4ccccc43)c2)c2cccc3c4ccccc4n(-c4ccccc4)c23)cc1] | 60% |
| Com 27-28 | 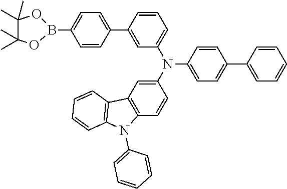 [SMILES: CC1(C)OB(c2ccc(-c3cccc(N(c4ccc(-c5ccccc5)cc4)c4ccc5c(c4)c4ccccc4n5-c4ccccc4)c3)cc2)OC1(C)C] | 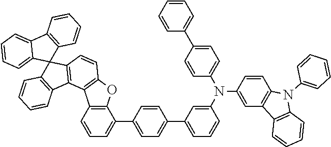 [SMILES: c1ccc(-c2ccc(N(c3cccc(-c4ccc(-c5cccc6c5oc5ccc7c(c56)-c5ccccc5C75c6ccccc6-c6ccccc65)cc4)c3)c3ccc4c(c3)c3ccccc3n4-c3ccccc3)cc2)cc1] | 52% |
| Com 27-29 | 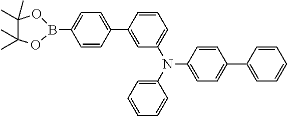 [SMILES: CC1(C)OB(c2ccc(-c3cccc(N(c4ccccc4)c4ccc(-c5ccccc5)cc4)c3)cc2)OC1(C)C] | 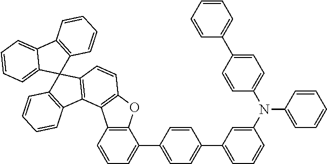 [SMILES: c1ccc(-c2ccc(N(c3ccccc3)c3cccc(-c4ccc(-c5cccc6c5oc5ccc7c(c56)-c5ccccc5C75c6ccccc6-c6ccccc65)cc4)c3)cc2)cc1] | 54% |
| Com 27-30 | 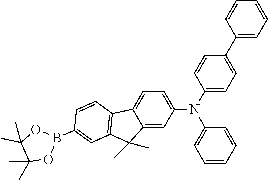 [SMILES: CC1(C)c2cc(B3OC(C)(C)C(C)(C)O3)ccc2-c2ccc(N(c3ccccc3)c3ccc(-c4ccccc4)cc3)cc21] | 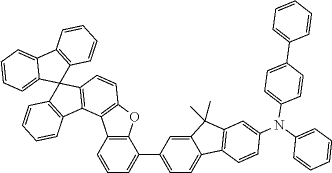 [SMILES: CC1(C)c2cc(-c3cccc4c3oc3ccc5c(c34)-c3ccccc3C53c4ccccc4-c4ccccc43)ccc2-c2ccc(N(c3ccccc3)c3ccc(-c4ccccc4)cc3)cc21] | 57% |
| Com 27-31 | 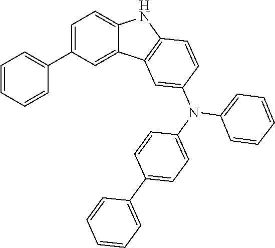 [SMILES: c1ccc(-c2ccc(N(c3ccccc3)c3ccc4[nH]c5ccc(-c6ccccc6)cc5c4c3)cc2)cc1] | 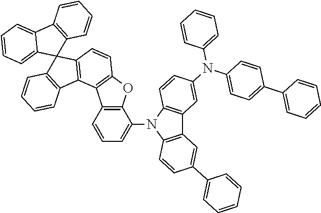 [SMILES: c1ccc(-c2ccc(N(c3ccccc3)c3ccc4c(c3)c3cc(-c5ccccc5)ccc3n4-c3cccc4c3oc3ccc5c(c34)-c3ccccc3C53c4ccccc4-c4ccccc43)cc2)cc1] | 71% |
| Com 27-32 | 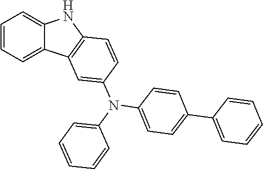 [SMILES: c1ccc(-c2ccc(N(c3ccccc3)c3ccc4[nH]c5ccccc5c4c3)cc2)cc1] | 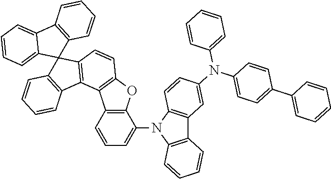 [SMILES: c1ccc(-c2ccc(N(c3ccccc3)c3ccc4c(c3)c3ccccc3n4-c3cccc4c3oc3ccc5c(c34)-c3ccccc3C53c4ccccc4-c4ccccc43)cc2)cc1] | 67% |
| Com 27-33 | 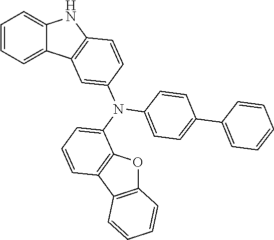 [SMILES: c1ccc(-c2ccc(N(c3ccc4[nH]c5ccccc5c4c3)c3cccc4c3oc3ccccc34)cc2)cc1] | 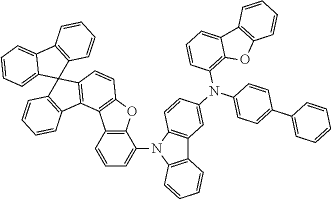 [SMILES: c1ccc(-c2ccc(N(c3ccc4c(c3)c3ccccc3n4-c3cccc4c3oc3ccc5c(c34)-c3ccccc3C53c4ccccc4-c4ccccc43)c3cccc4c3oc3ccccc34)cc2)cc1] | 68% |
| Com 27-34 | 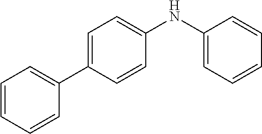 [SMILES: c1ccc(Nc2ccc(-c3ccccc3)cc2)cc1] | 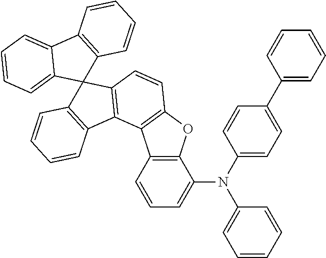 [SMILES: c1ccc(-c2ccc(N(c3ccccc3)c3cccc4c3oc3ccc5c(c34)-c3ccccc3C53c4ccccc4-c4ccccc43)cc2)cc1] | 65% |
| Com 27-35 | 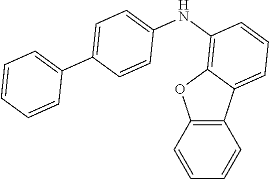 [SMILES: c1ccc(-c2ccc(Nc3cccc4c3oc3ccccc34)cc2)cc1] | 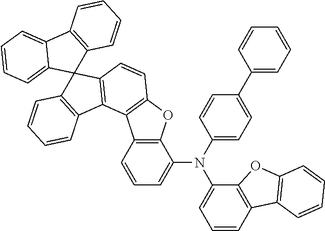 [SMILES: c1ccc(-c2ccc(N(c3cccc4c3oc3ccccc34)c3cccc4c3oc3ccc5c(c34)-c3ccccc3C53c4ccccc4-c4ccccc43)cc2)cc1] | 81% |
| Com 27-36 | 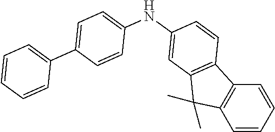 [SMILES: CC1(C)c2ccccc2-c2ccc(Nc3ccc(-c4ccccc4)cc3)cc21] | 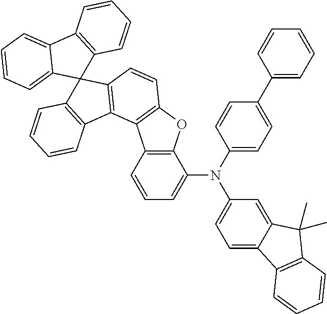 [SMILES: CC1(C)c2ccccc2-c2ccc(N(c3ccc(-c4ccccc4)cc3)c3cccc4c3oc3ccc5c(c34)-c3ccccc3C53c4ccccc4-c4ccccc43)cc21] | 85% |
| Com 27-37 | 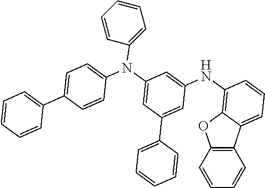 [SMILES: c1ccc(-c2ccc(N(c3ccccc3)c3cc(Nc4cccc5c4oc4ccccc45)cc(-c4ccccc4)c3)cc2)cc1] | 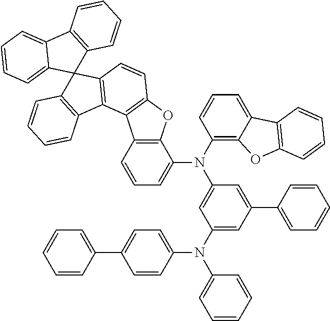 [SMILES: c1ccc(-c2ccc(N(c3ccccc3)c3cc(-c4ccccc4)cc(N(c4cccc5c4oc4ccccc45)c4cccc5c4oc4ccc6c(c45)-c4ccccc4C64c5ccccc5-c5ccccc54)c3)cc2)cc1] | 82% |
| Com 27-38 | 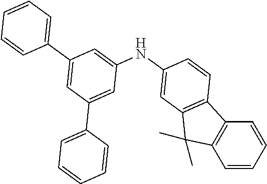 [SMILES: CC1(C)c2ccccc2-c2ccc(Nc3cc(-c4ccccc4)cc(-c4ccccc4)c3)cc21] | 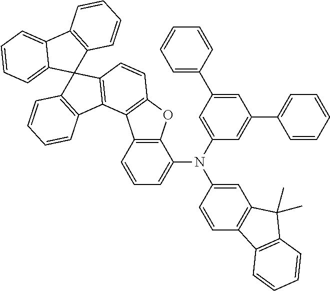 [SMILES: CC1(C)c2ccccc2-c2ccc(N(c3cc(-c4ccccc4)cc(-c4ccccc4)c3)c3cccc4c3oc3ccc5c(c34)-c3ccccc3C53c4ccccc4-c4ccccc43)cc21] | 82% |
| Com 27-39 | 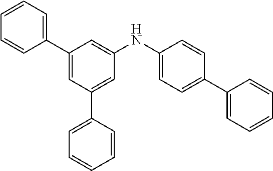 [SMILES: c1ccc(-c2ccc(Nc3cc(-c4ccccc4)cc(-c4ccccc4)c3)cc2)cc1] | 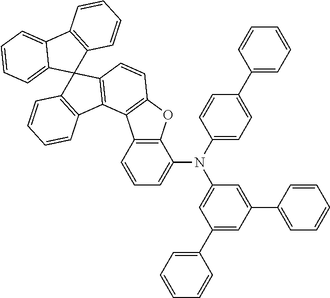 [SMILES: c1ccc(-c2ccc(N(c3cc(-c4ccccc4)cc(-c4ccccc4)c3)c3cccc4c3oc3ccc5c(c34)-c3ccccc3C53c4ccccc4-c4ccccc43)cc2)cc1] | 81% |
| Com 27-40 | 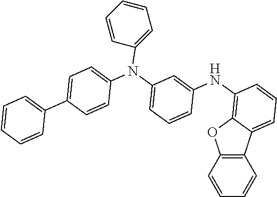 [SMILES: c1ccc(-c2ccc(N(c3ccccc3)c3cccc(Nc4cccc5c4oc4ccccc45)c3)cc2)cc1] | 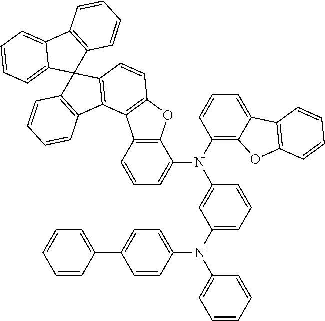 [SMILES: c1ccc(-c2ccc(N(c3ccccc3)c3cccc(N(c4cccc5c4oc4ccccc45)c4cccc5c4oc4ccc6c(c45)-c4ccccc4C64c5ccccc5-c5ccccc54)c3)cc2)cc1] | 80% |
| Com 27-41 | 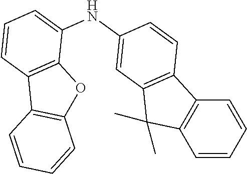 [SMILES: CC1(C)c2ccccc2-c2ccc(Nc3cccc4c3oc3ccccc34)cc21] | 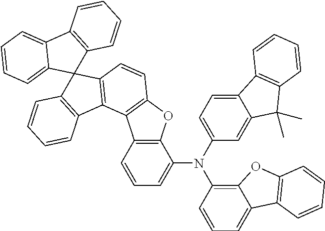 [SMILES: CC1(C)c2ccccc2-c2ccc(N(c3cccc4c3oc3ccccc34)c3cccc4c3oc3ccc5c(c34)-c3ccccc3C53c4ccccc4-c4ccccc43)cc21] | 70% |
| Com 27-42 | 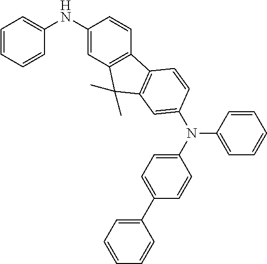 [SMILES: CC1(C)c2cc(Nc3ccccc3)ccc2-c2ccc(N(c3ccccc3)c3ccc(-c4ccccc4)cc3)cc21] | 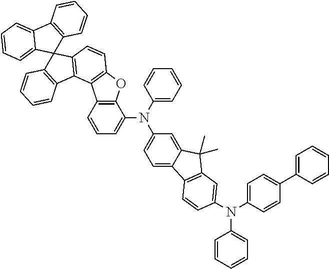 [SMILES: CC1(C)c2cc(N(c3ccccc3)c3ccc(-c4ccccc4)cc3)ccc2-c2ccc(N(c3ccccc3)c3cccc4c3oc3ccc5c(c34)-c3ccccc3C53c4ccccc4-c4ccccc43)cc21] | 88% |
| Com 27-43 | 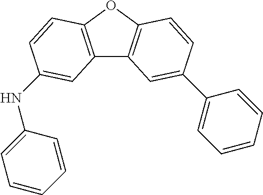 [SMILES: c1ccc(Nc2ccc3oc4ccc(-c5ccccc5)cc4c3c2)cc1] | 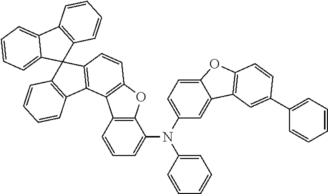 [SMILES: c1ccc(-c2ccc3oc4ccc(N(c5ccccc5)c5cccc6c5oc5ccc7c(c56)-c5ccccc5C75c6ccccc6-c6ccccc65)cc4c3c2)cc1] | 84% |
| Com 27-44 | 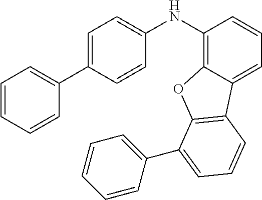 [SMILES: c1ccc(-c2ccc(Nc3cccc4c3oc3c(-c5ccccc5)cccc34)cc2)cc1] | 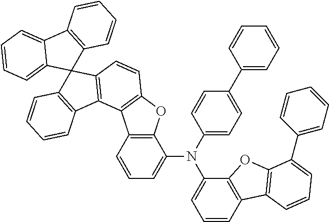 [SMILES: c1ccc(-c2ccc(N(c3cccc4c3oc3c(-c5ccccc5)cccc34)c3cccc4c3oc3ccc5c(c34)-c3ccccc3C53c4ccccc4-c4ccccc43)cc2)cc1] | 89% |
| Com 27-45 | 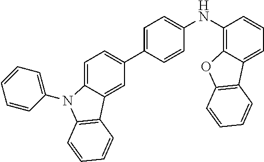 [SMILES: c1ccc(-n2c3ccccc3c3cc(-c4ccc(Nc5cccc6c5oc5ccccc56)cc4)ccc32)cc1] | 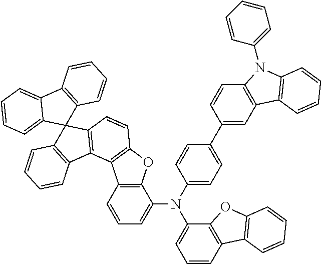 [SMILES: c1ccc(-n2c3ccccc3c3cc(-c4ccc(N(c5cccc6c5oc5ccccc56)c5cccc6c5oc5ccc7c(c56)-c5ccccc5C75c6ccccc6-c6ccccc65)cc4)ccc32)cc1] | 83% |
| Com 27-46 | 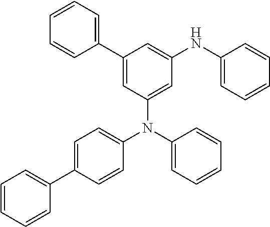 [SMILES: c1ccc(Nc2cc(-c3ccccc3)cc(N(c3ccccc3)c3ccc(-c4ccccc4)cc3)c2)cc1] | 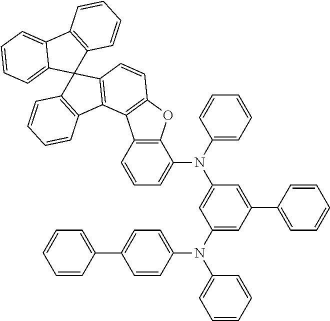 [SMILES: c1ccc(-c2ccc(N(c3ccccc3)c3cc(-c4ccccc4)cc(N(c4ccccc4)c4cccc5c4oc4ccc6c(c45)-c4ccccc4C64c5ccccc5-c5ccccc54)c3)cc2)cc1] | 71% |
| Com 27-47 | 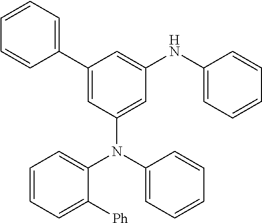 [SMILES: c1ccc(Nc2cc(-c3ccccc3)cc(N(c3ccccc3)c3ccccc3-c3ccccc3)c2)cc1] | 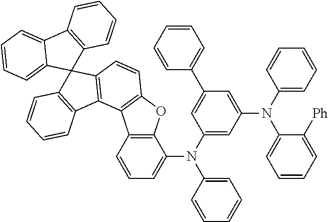 [SMILES: c1ccc(-c2cc(N(c3ccccc3)c3ccccc3-c3ccccc3)cc(N(c3ccccc3)c3cccc4c3oc3ccc5c(c34)-c3ccccc3C53c4ccccc4-c4ccccc43)c2)cc1] | 75% |
| Com 27-48 | 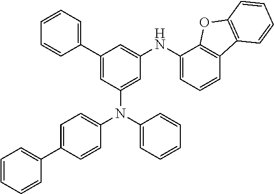 [SMILES: c1ccc(-c2ccc(N(c3ccccc3)c3cc(Nc4cccc5c4oc4ccccc45)cc(-c4ccccc4)c3)cc2)cc1] | 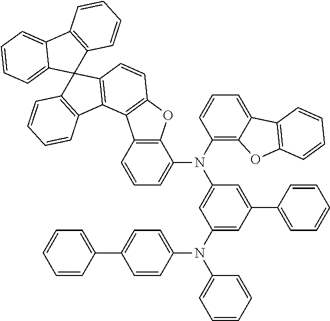 [SMILES: c1ccc(-c2ccc(N(c3ccccc3)c3cc(-c4ccccc4)cc(N(c4cccc5c4oc4ccccc45)c4cccc5c4oc4ccc6c(c45)-c4ccccc4C64c5ccccc5-c5ccccc54)c3)cc2)cc1] | 65% |
| Com 27-49 | 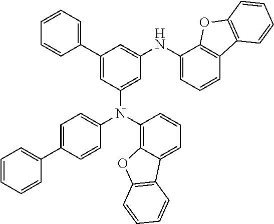 [SMILES: c1ccc(-c2ccc(N(c3cc(Nc4cccc5c4oc4ccccc45)cc(-c4ccccc4)c3)c3cccc4c3oc3ccccc34)cc2)cc1] | 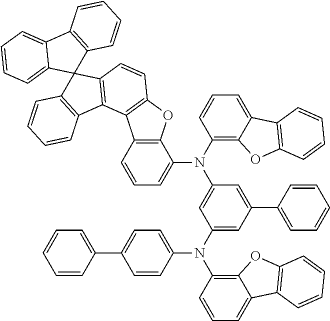 [SMILES: c1ccc(-c2ccc(N(c3cc(-c4ccccc4)cc(N(c4cccc5c4oc4ccccc45)c4cccc5c4oc4ccc6c(c45)-c4ccccc4C64c5ccccc5-c5ccccc54)c3)c3cccc4c3oc3ccccc34)cc2)cc1] | 69% |
| Com 27-50 | 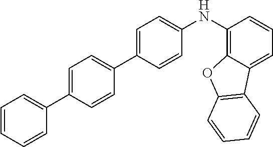 [SMILES: c1ccc(-c2ccc(-c3ccc(Nc4cccc5c4oc4ccccc45)cc3)cc2)cc1] | 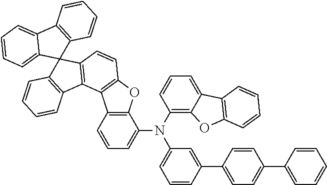 [SMILES: c1ccc(-c2ccc(-c3cccc(N(c4cccc5c4oc4ccccc45)c4cccc5c4oc4ccc6c(c45)-c4ccccc4C64c5ccccc5-c5ccccc54)c3)cc2)cc1] | 66% |

### [Preparation Example 6] Preparation of Compound F-1

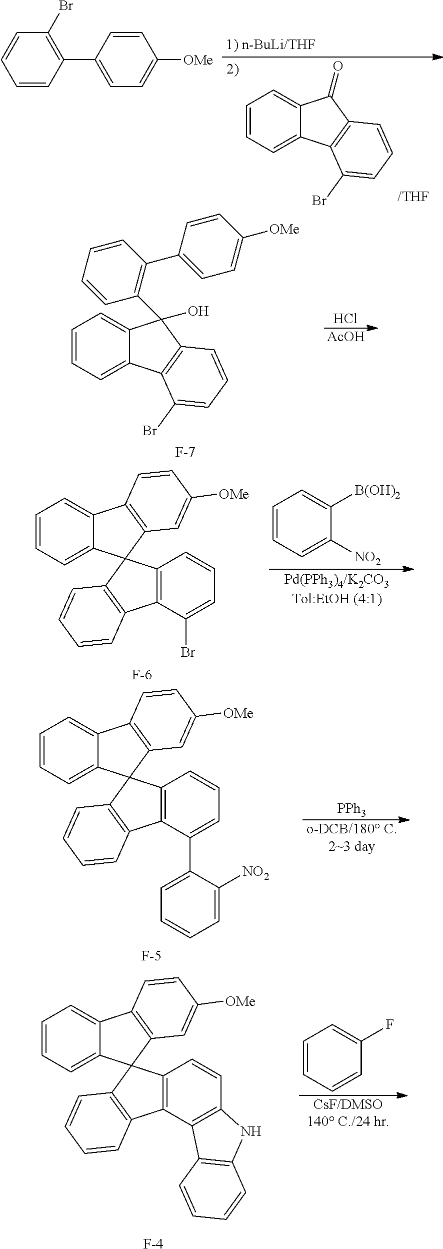

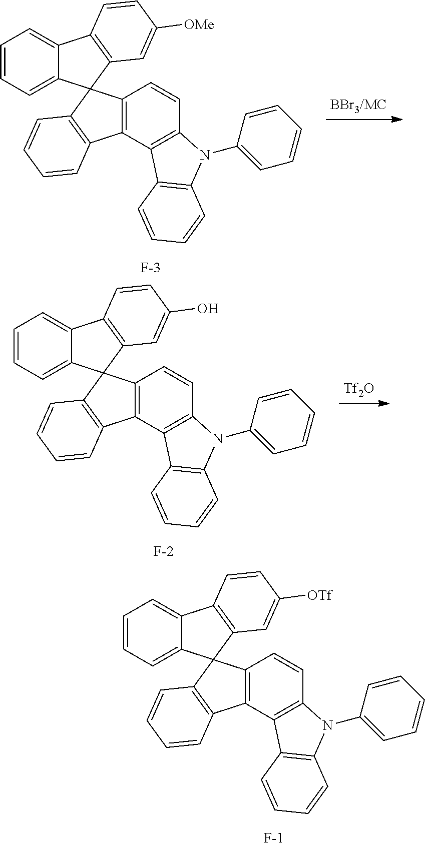

Preparation of Compound F-6

A compound F-6 was prepared in the same manner as that of Preparation Example 1.

Preparation of Compound F-5

10 g (23.51 mmol) of the compound F-6 was dissolved in 100 mL of toluene, 4.77 g (28.21 mmol) of 2-nitrophenylboronic acid, 1.36 g (1.18 mmol) of Pd(PPh₃)₄, and 8.12 g (58.78 mmol) of K₂CO₃ were added, and stirring was performed. 25 mL of ethanol and 25 mL of water were added thereto, and the mixed solution was refluxed at 130° C. for 24 hours. After the reaction was completed, extraction was performed using MC and H₂O, the solvent was removed, and then purification was performed by column chromatography with Hex and MC, thereby obtaining 9.67 g (88%) of a target compound F-5.

Preparation of Compound F-4

10 g (21.39 mmol) of the compound F-5 was dissolved in 110 mL of o-DCB, 16.83 g (64.17 mmol) of triphenylphosphine (PPh₃) was added, and stirring was performed under reflux at 180° C. for 2 days. After the reaction was completed, a sodium bicarbonate (NaHCO₃) aqueous solution was added to stop the reaction, extraction was performed using MC and H₂O, the solvent was removed, and then purification was performed by column chromatography with Hex and MC, thereby obtaining 8.01 g (86%) of a target compound F-4.

Preparation of Compound F-3

10 g (22.96 mmol) of the compound F-4 was dissolved in 120 mL of DMSO, 2.65 g (27.55 mmol) of fluorobenzene and 4.19 g (27.55 mmol) of cesium fluoride (CsF) were added thereto, and stirring was performed under reflux at 140° C. for 24 hours. After the reaction was completed, the reaction solution was cooled to room temperature, the produced solid was filtered and then washed with water, ethanol, Hex, and EA, and then recrystallization was performed with Hex/EA, thereby obtaining 9.99 g (85%) of a target compound F-3.

Preparation of Compound F-2

10 g (19.55 mmol) of the compound F-3 was dissolved in 110 mL of MC, and then the temperature was lowered to 0° C. 24 mL (23.46 mmol) of tribromoboron (1.0 M solution in MC) was slowly added dropwise thereto, and stirring was performed at the same temperature for 30 minutes. The temperature of the reaction solution was slowly raised to room temperature, and stirring was additionally performed at room temperature for 24 hours. After the reaction was completed, extraction was performed with MC and a NaHCO₃ aqueous solution, the MC layer was collected, the solvent was removed, and the produced solid was recrystallized using MC and Hex, thereby obtaining 7.00 g (72%) of a target compound F-2.

Preparation of Compound F-1

10 g (20.10 mmol) of the compound F-2 was dissolved in 120 mL of THF, 6.1 mL (44.22 mmol) of TEA and 4.1 mL (24.12 mmol) of triflic anhydride were added, and then stirring was performed at room temperature for 24 hours. After the reaction was completed, extraction was performed using MC and a NaHCO₃ aqueous solution, the MC layer was collected, the solvent was removed, and the produced solid was recrystallized and purified using DMF and Hex, thereby obtaining 9.37 g (74%) of a target compound F-1.

### [Example 6] Preparation of Compounds P11 and P12

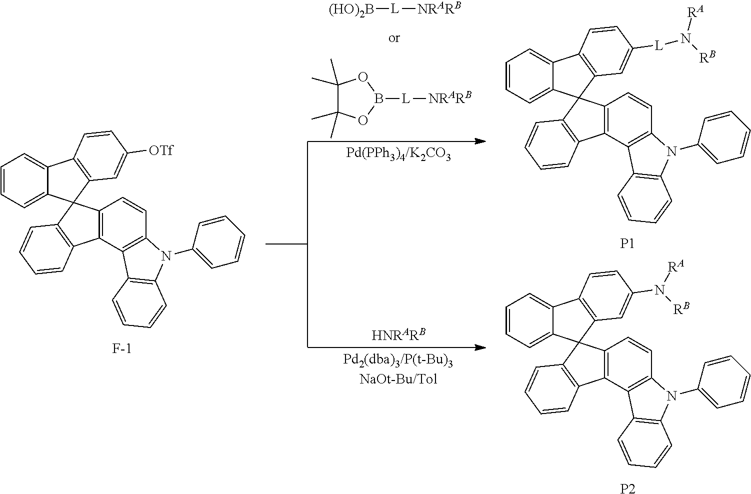

Preparation of Compound P11

A target compound P11 was obtained through a Suzuki coupling reaction using the compound F-1, a substituted boronic acid or boronic ester compound, and a palladium catalyst.

Preparation of Compound P12

A target compound P12 was obtained through palladium-amination using the compound F-1, a secondary amine compound, and a palladium catalyst.

The structures of the substituted boronic acid, boronic ester, and secondary amine compound as the reactants and the structures and yields of the prepared compounds P11 and P12 are shown in Table 6.

TABLE 6

| Comp | | | |
|---|---|---|---|
| ound | | | |
| No. | Reactant | Product (P11, P12) | Yield |
| Com 36-1 | 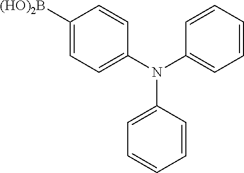 [SMILES: OB(O)c1ccc(N(c2ccccc2)c2ccccc2)cc1] | 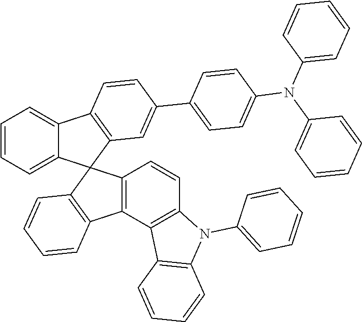 [SMILES: c1ccc(N(c2ccccc2)c2ccc(-c3ccc4c(c3)C3(c5ccccc5-4)c4ccccc4-c4c3ccc3c4c4ccccc4n3-c3ccccc3)cc2)cc1] | 71% |
| Com 36-2 | 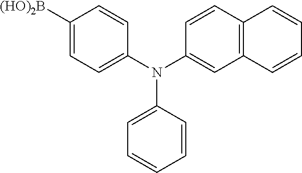 [SMILES: OB(O)c1ccc(N(c2ccccc2)c2ccc3ccccc3c2)cc1] | 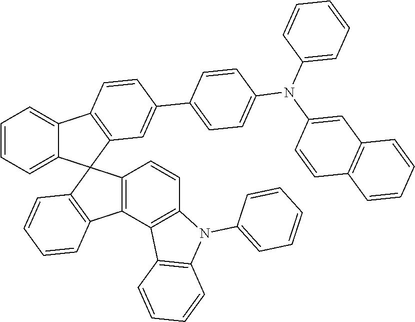 [SMILES: c1ccc(N(c2ccc(-c3ccc4c(c3)C3(c5ccccc5-4)c4ccccc4-c4c3ccc3c4c4ccccc4n3-c3ccccc3)cc2)c2ccc3ccccc3c2)cc1] | 83% |
| Com 36-3 | 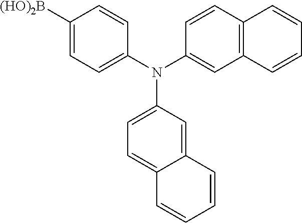 [SMILES: OB(O)c1ccc(N(c2ccc3ccccc3c2)c2ccc3ccccc3c2)cc1] | 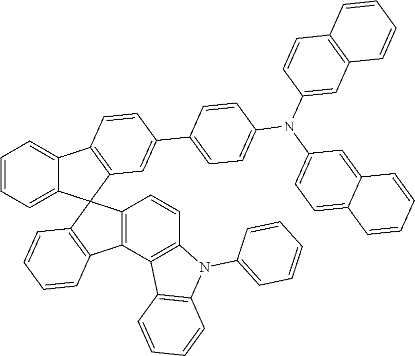 [SMILES: c1ccc(-n2c3ccccc3c3c4c(ccc32)C2(c3ccccc3-c3ccc(-c5ccc(N(c6ccc7ccccc7c6)c6ccc7ccccc7c6)cc5)cc32)c2ccccc2-4)cc1] | 75% |
| Com 36-4 | 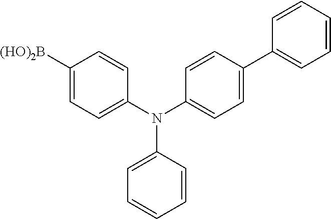 [SMILES: OB(O)c1ccc(N(c2ccccc2)c2ccc(-c3ccccc3)cc2)cc1] | 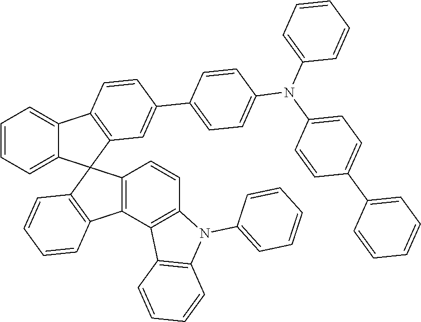 [SMILES: c1ccc(-c2ccc(N(c3ccccc3)c3ccc(-c4ccc5c(c4)C4(c6ccccc6-5)c5ccccc5-c5c4ccc4c5c5ccccc5n4-c4ccccc4)cc3)cc2)cc1] | 75% |
| Com 36-5 | 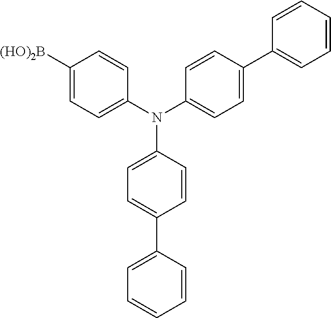 [SMILES: OB(O)c1ccc(N(c2ccc(-c3ccccc3)cc2)c2ccc(-c3ccccc3)cc2)cc1] | 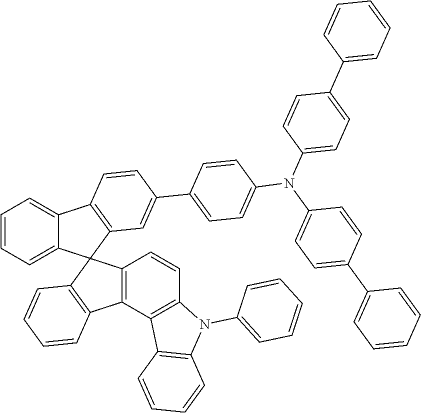 [SMILES: c1ccc(-c2ccc(N(c3ccc(-c4ccccc4)cc3)c3ccc(-c4ccc5c(c4)C4(c6ccccc6-5)c5ccccc5-c5c4ccc4c5c5ccccc5n4-c4ccccc4)cc3)cc2)cc1] | 65% |
| Com 36-6 | 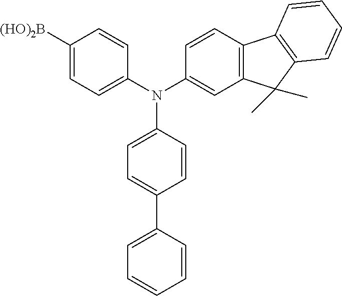 [SMILES: CC1(C)c2ccccc2-c2ccc(N(c3ccc(B(O)O)cc3)c3ccc(-c4ccccc4)cc3)cc21] | 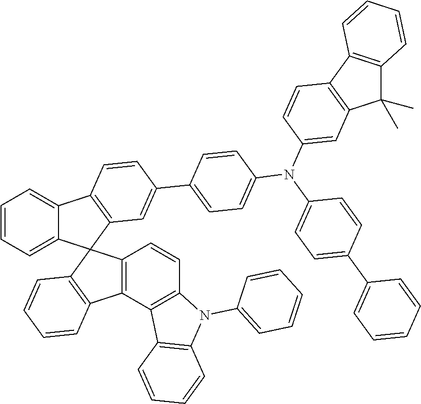 [SMILES: CC1(C)c2ccccc2-c2ccc(N(c3ccc(-c4ccccc4)cc3)c3ccc(-c4ccc5c(c4)C4(c6ccccc6-5)c5ccccc5-c5c4ccc4c5c5ccccc5n4-c4ccccc4)cc3)cc21] | 71% |
| Com 36-7 | 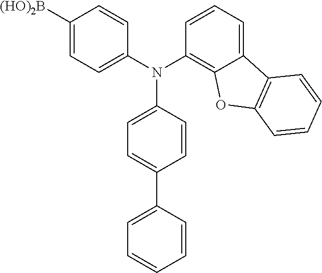 [SMILES: OB(O)c1ccc(N(c2ccc(-c3ccccc3)cc2)c2cccc3c2oc2ccccc23)cc1] | 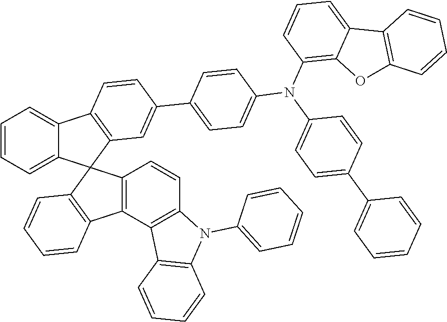 [SMILES: c1ccc(-c2ccc(N(c3ccc(-c4ccc5c(c4)C4(c6ccccc6-5)c5ccccc5-c5c4ccc4c5c5ccccc5n4-c4ccccc4)cc3)c3cccc4c3oc3ccccc34)cc2)cc1] | 62% |
| Com 36-8 | 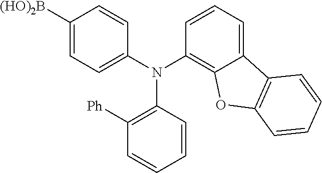 [SMILES: OB(O)c1ccc(N(c2ccccc2-c2ccccc2)c2cccc3c2oc2ccccc23)cc1] | 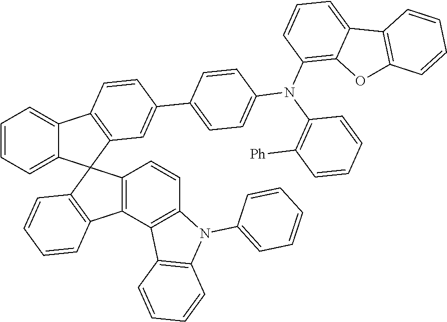 [SMILES: c1ccc(-c2ccccc2N(c2ccc(-c3ccc4c(c3)C3(c5ccccc5-4)c4ccccc4-c4c3ccc3c4c4ccccc4n3-c3ccccc3)cc2)c2cccc3c2oc2ccccc23)cc1] | 61% |
| Com 36-9 | 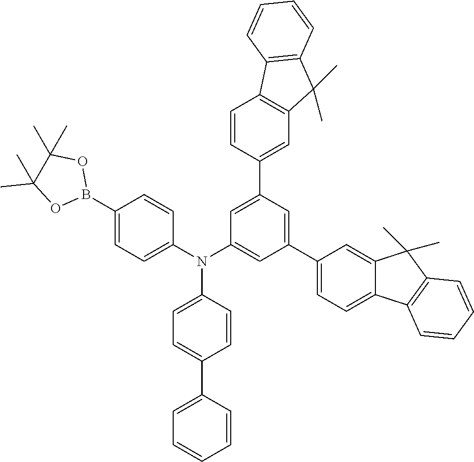 [SMILES: CC1(C)c2ccccc2-c2ccc(-c3cc(-c4ccc5c(c4)C(C)(C)c4ccccc4-5)cc(N(c4ccc(B5OC(C)(C)C(C)(C)O5)cc4)c4ccc(-c5ccccc5)cc4)c3)cc21] | 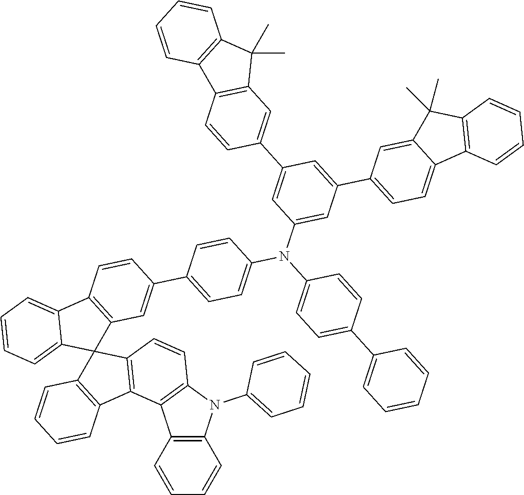 [SMILES: CC1(C)c2ccccc2-c2ccc(-c3cc(-c4ccc5c(c4)C(C)(C)c4ccccc4-5)cc(N(c4ccc(-c5ccccc5)cc4)c4ccc(-c5ccc6c(c5)C5(c7ccccc7-6)c6ccccc6-c6c5ccc5c6c6ccccc6n5-c5ccccc5)cc4)c3)cc21] | 66% |
| Com 36-10 | 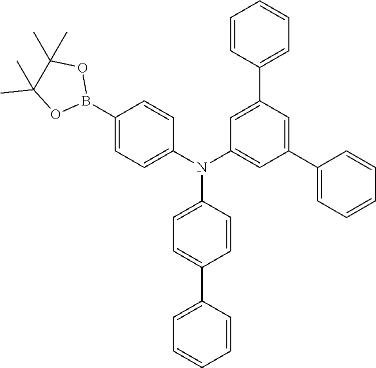 [SMILES: CC1(C)OB(c2ccc(N(c3ccc(-c4ccccc4)cc3)c3cc(-c4ccccc4)cc(-c4ccccc4)c3)cc2)OC1(C)C] | 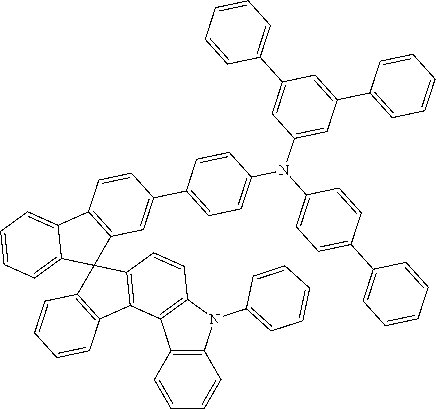 [SMILES: c1ccc(-c2ccc(N(c3ccc(-c4ccc5c(c4)C4(c6ccccc6-5)c5ccccc5-c5c4ccc4c5c5ccccc5n4-c4ccccc4)cc3)c3cc(-c4ccccc4)cc(-c4ccccc4)c3)cc2)cc1] | 63% |
| Com 36-11 | 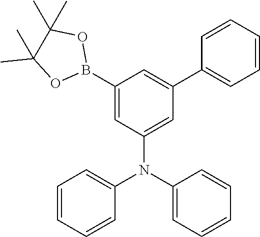 [SMILES: CC1(C)OB(c2cc(-c3ccccc3)cc(N(c3ccccc3)c3ccccc3)c2)OC1(C)C] | 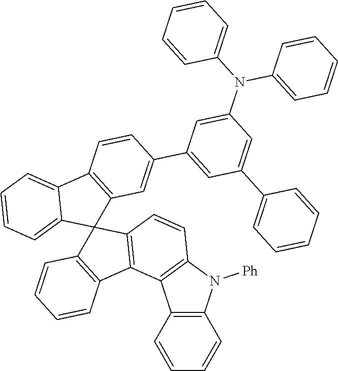 [SMILES: c1ccc(-c2cc(-c3ccc4c(c3)C3(c5ccccc5-4)c4ccccc4-c4c3ccc3c4c4ccccc4n3-c3ccccc3)cc(N(c3ccccc3)c3ccccc3)c2)cc1] | 71% |
| Com 36-12 | 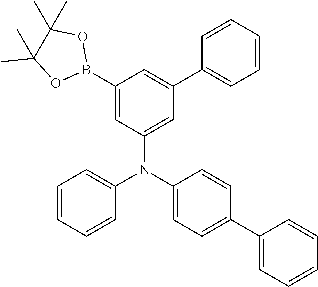 [SMILES: CC1(C)OB(c2cc(-c3ccccc3)cc(N(c3ccccc3)c3ccc(-c4ccccc4)cc3)c2)OC1(C)C] | 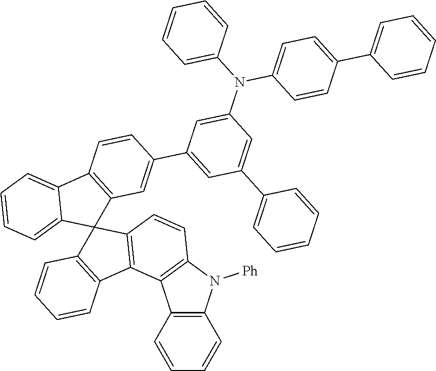 [SMILES: c1ccc(-c2ccc(N(c3ccccc3)c3cc(-c4ccccc4)cc(-c4ccc5c(c4)C4(c6ccccc6-5)c5ccccc5-c5c4ccc4c5c5ccccc5n4-c4ccccc4)c3)cc2)cc1] | 65% |
| Com 36-13 | 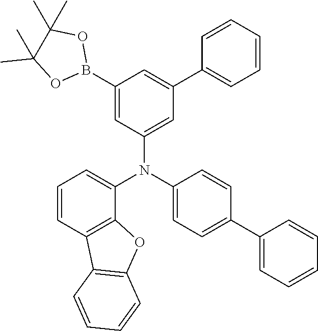 [SMILES: CC1(C)OB(c2cc(-c3ccccc3)cc(N(c3ccc(-c4ccccc4)cc3)c3cccc4c3oc3ccccc34)c2)OC1(C)C] | 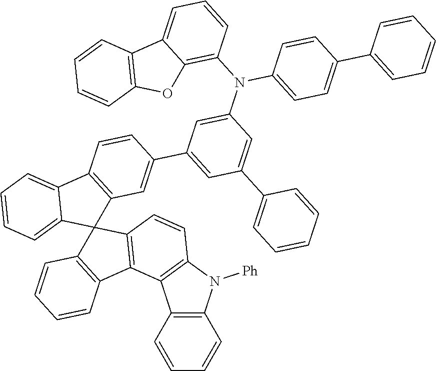 [SMILES: c1ccc(-c2ccc(N(c3cc(-c4ccccc4)cc(-c4ccc5c(c4)C4(c6ccccc6-5)c5ccccc5-c5c4ccc4c5c5ccccc5n4-c4ccccc4)c3)c3cccc4c3oc3ccccc34)cc2)cc1] | 65% |
| Com 36-14 | 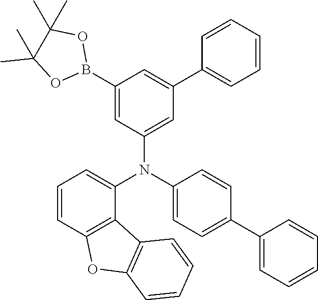 [SMILES: CC1(C)OB(c2cc(-c3ccccc3)cc(N(c3ccc(-c4ccccc4)cc3)c3cccc4oc5ccccc5c34)c2)OC1(C)C] | 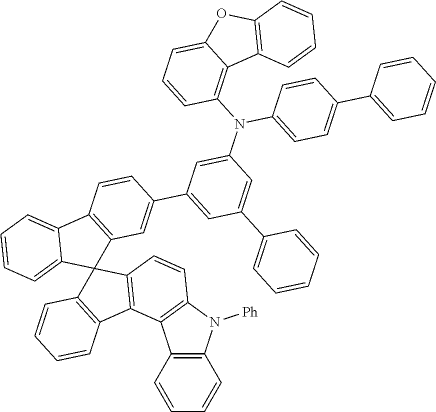 [SMILES: c1ccc(-c2ccc(N(c3cc(-c4ccccc4)cc(-c4ccc5c(c4)C4(c6ccccc6-5)c5ccccc5-c5c4ccc4c5c5ccccc5n4-c4ccccc4)c3)c3cccc4oc5ccccc5c34)cc2)cc1] | 58% |
| Com 36-15 | 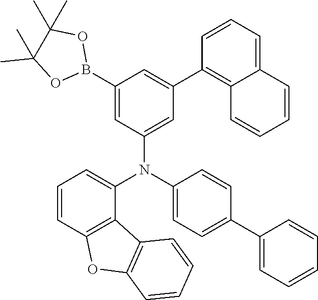 [SMILES: CC1(C)OB(c2cc(-c3cccc4ccccc34)cc(N(c3ccc(-c4ccccc4)cc3)c3cccc4oc5ccccc5c34)c2)OC1(C)C] | 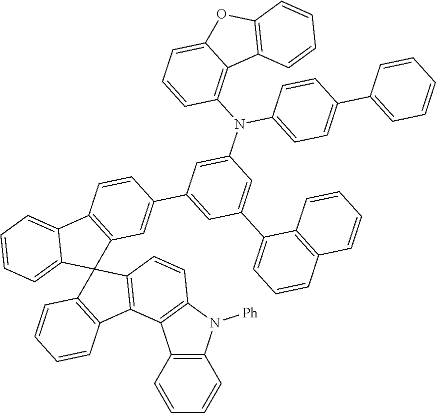 [SMILES: c1ccc(-c2ccc(N(c3cc(-c4ccc5c(c4)C4(c6ccccc6-5)c5ccccc5-c5c4ccc4c5c5ccccc5n4-c4ccccc4)cc(-c4cccc5ccccc45)c3)c3cccc4oc5ccccc5c34)cc2)cc1] | 74% |
| Com 36-16 | 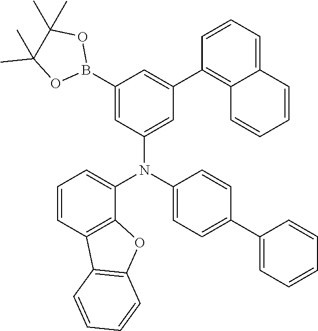 [SMILES: CC1(C)OB(c2cc(-c3cccc4ccccc34)cc(N(c3ccc(-c4ccccc4)cc3)c3cccc4c3oc3ccccc34)c2)OC1(C)C] | 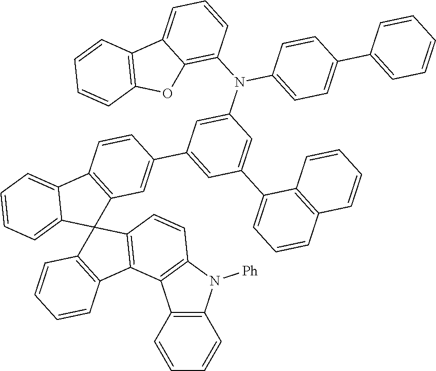 [SMILES: c1ccc(-c2ccc(N(c3cc(-c4ccc5c(c4)C4(c6ccccc6-5)c5ccccc5-c5c4ccc4c5c5ccccc5n4-c4ccccc4)cc(-c4cccc5ccccc45)c3)c3cccc4c3oc3ccccc34)cc2)cc1] | 58% |
| Com 36-17 | 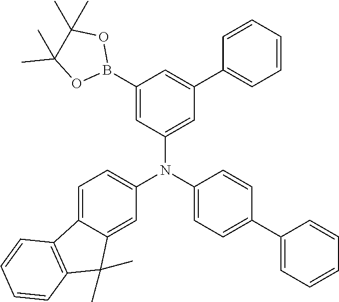 [SMILES: CC1(C)c2ccccc2-c2ccc(N(c3ccc(-c4ccccc4)cc3)c3cc(B4OC(C)(C)C(C)(C)O4)cc(-c4ccccc4)c3)cc21] | 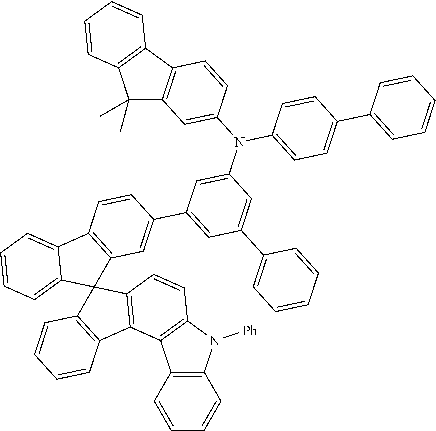 [SMILES: CC1(C)c2ccccc2-c2ccc(N(c3ccc(-c4ccccc4)cc3)c3cc(-c4ccccc4)cc(-c4ccc5c(c4)C4(c6ccccc6-5)c5ccccc5-c5c4ccc4c5c5ccccc5n4-c4ccccc4)c3)cc21] | 65% |
| Com 36-18 | 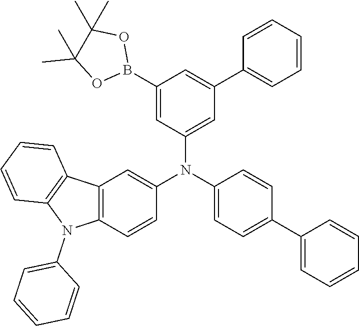 [SMILES: CC1(C)OB(c2cc(-c3ccccc3)cc(N(c3ccc(-c4ccccc4)cc3)c3ccc4c(c3)c3ccccc3n4-c3ccccc3)c2)OC1(C)C] | 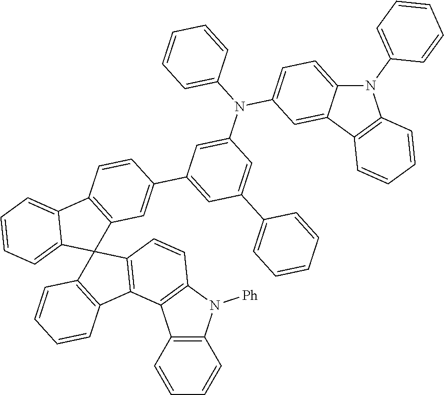 [SMILES: c1ccc(-c2cc(-c3ccc4c(c3)C3(c5ccccc5-4)c4ccccc4-c4c3ccc3c4c4ccccc4n3-c3ccccc3)cc(N(c3ccccc3)c3ccc4c(c3)c3ccccc3n4-c3ccccc3)c2)cc1] | 61% |
| Com 36-19 | 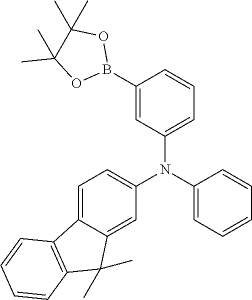 [SMILES: CC1(C)c2ccccc2-c2ccc(N(c3ccccc3)c3cccc(B4OC(C)(C)C(C)(C)O4)c3)cc21] | 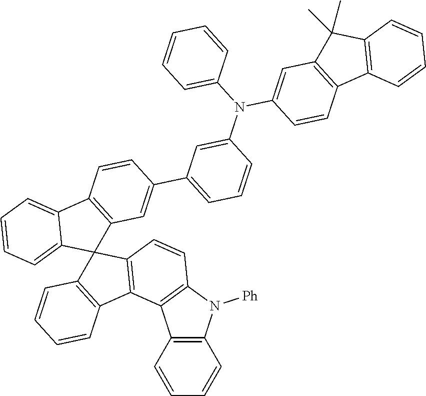 [SMILES: CC1(C)c2ccccc2-c2ccc(N(c3ccccc3)c3cccc(-c4ccc5c(c4)C4(c6ccccc6-5)c5ccccc5-c5c4ccc4c5c5ccccc5n4-c4ccccc4)c3)cc21] | 77% |
| Com 36-20 | 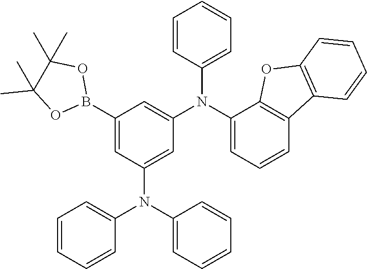 [SMILES: CC1(C)OB(c2cc(N(c3ccccc3)c3ccccc3)cc(N(c3ccccc3)c3cccc4c3oc3ccccc34)c2)OC1(C)C] | 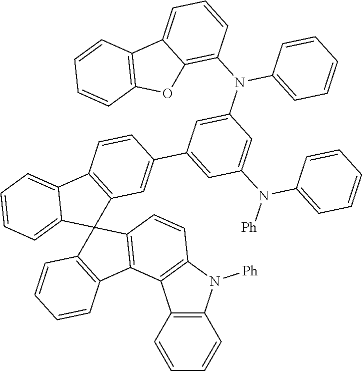 [SMILES: c1ccc(N(c2ccccc2)c2cc(-c3ccc4c(c3)C3(c5ccccc5-4)c4ccccc4-c4c3ccc3c4c4ccccc4n3-c3ccccc3)cc(N(c3ccccc3)c3cccc4c3oc3ccccc34)c2)cc1] | 69% |
| Com 36-21 | 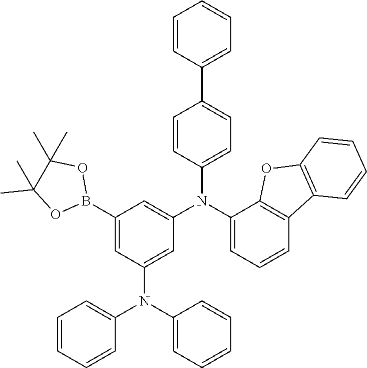 [SMILES: CC1(C)OB(c2cc(N(c3ccccc3)c3ccccc3)cc(N(c3ccc(-c4ccccc4)cc3)c3cccc4c3oc3ccccc34)c2)OC1(C)C] | 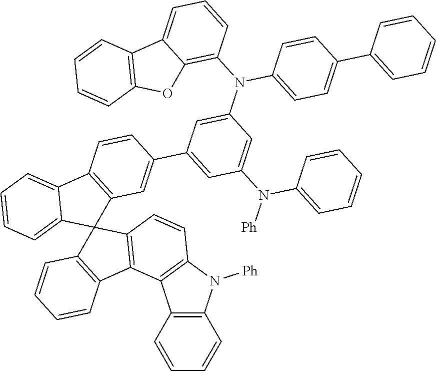 [SMILES: c1ccc(-c2ccc(N(c3cc(-c4ccc5c(c4)C4(c6ccccc6-5)c5ccccc5-c5c4ccc4c5c5ccccc5n4-c4ccccc4)cc(N(c4ccccc4)c4ccccc4)c3)c3cccc4c3oc3ccccc34)cc2)cc1] | 81% |
| Com 36-22 | 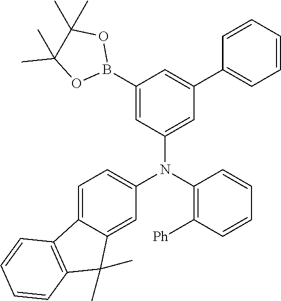 [SMILES: CC1(C)c2ccccc2-c2ccc(N(c3cc(B4OC(C)(C)C(C)(C)O4)cc(-c4ccccc4)c3)c3ccccc3-c3ccccc3)cc21] | 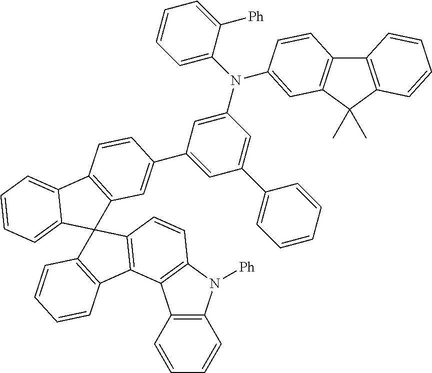 [SMILES: CC1(C)c2ccccc2-c2ccc(N(c3cc(-c4ccccc4)cc(-c4ccc5c(c4)C4(c6ccccc6-5)c5ccccc5-c5c4ccc4c5c5ccccc5n4-c4ccccc4)c3)c3ccccc3-c3ccccc3)cc21] | 75% |
| Com 36-23 | 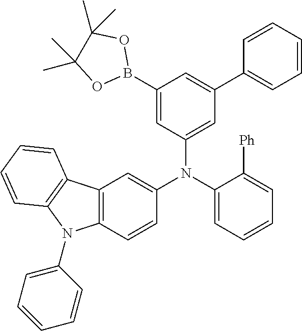 [SMILES: CC1(C)OB(c2cc(-c3ccccc3)cc(N(c3ccc4c(c3)c3ccccc3n4-c3ccccc3)c3ccccc3-c3ccccc3)c2)OC1(C)C] | 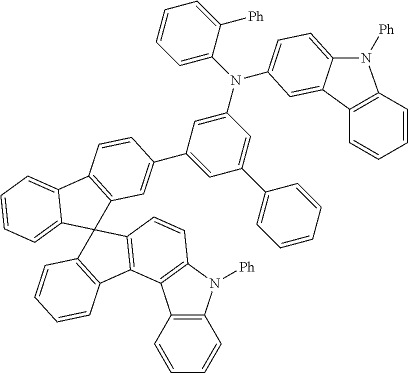 [SMILES: c1ccc(-c2cc(-c3ccc4c(c3)C3(c5ccccc5-4)c4ccccc4-c4c3ccc3c4c4ccccc4n3-c3ccccc3)cc(N(c3ccc4c(c3)c3ccccc3n4-c3ccccc3)c3ccccc3-c3ccccc3)c2)cc1] | 80% |
| Com 36-24 | 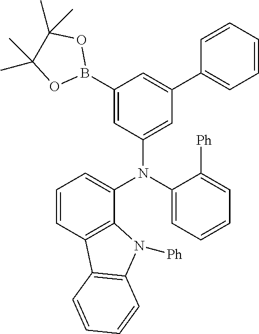 [SMILES: CC1(C)OB(c2cc(-c3ccccc3)cc(N(c3ccccc3-c3ccccc3)c3cccc4c5ccccc5n(-c5ccccc5)c34)c2)OC1(C)C] | 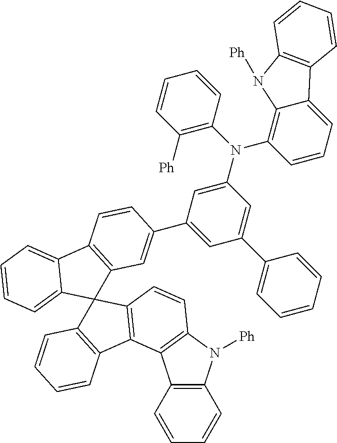 [SMILES: c1ccc(-c2cc(-c3ccc4c(c3)C3(c5ccccc5-4)c4ccccc4-c4c3ccc3c4c4ccccc4n3-c3ccccc3)cc(N(c3ccccc3-c3ccccc3)c3cccc4c5ccccc5n(-c5ccccc5)c34)c2)cc1] | 62% |
| Com 36-25 | 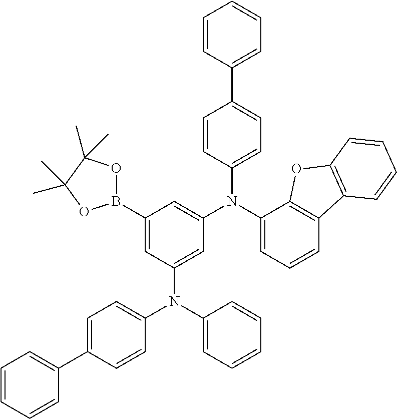 [SMILES: CC1(C)OB(c2cc(N(c3ccccc3)c3ccc(-c4ccccc4)cc3)cc(N(c3ccc(-c4ccccc4)cc3)c3cccc4c3oc3ccccc34)c2)OC1(C)C] | 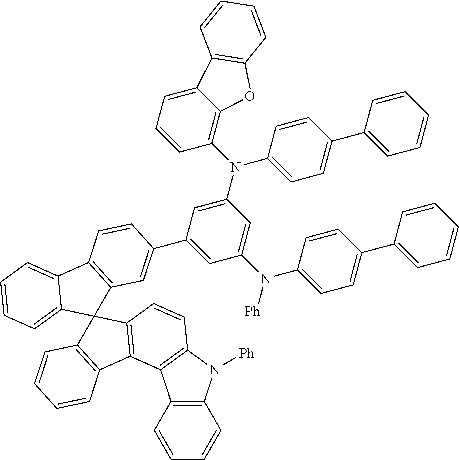 [SMILES: c1ccc(-c2ccc(N(c3ccccc3)c3cc(-c4ccc5c(c4)C4(c6ccccc6-5)c5ccccc5-c5c4ccc4c5c5ccccc5n4-c4ccccc4)cc(N(c4ccc(-c5ccccc5)cc4)c4cccc5c4oc4ccccc45)c3)cc2)cc1] | 61% |
| Com 36-26 | 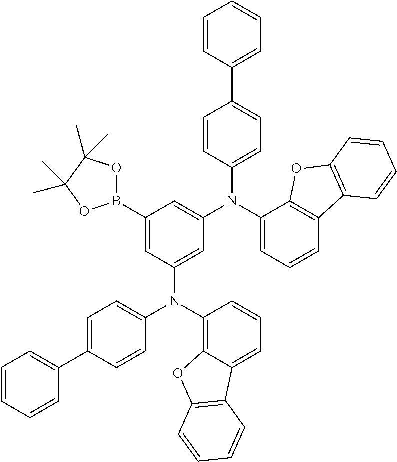 [SMILES: CC1(C)OB(c2cc(N(c3ccc(-c4ccccc4)cc3)c3cccc4c3oc3ccccc34)cc(N(c3ccc(-c4ccccc4)cc3)c3cccc4c3oc3ccccc34)c2)OC1(C)C] | 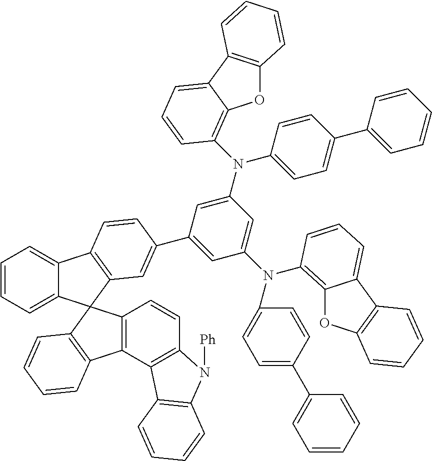 [SMILES: c1ccc(-c2ccc(N(c3cc(-c4ccc5c(c4)C4(c6ccccc6-5)c5ccccc5-c5c4ccc4c5c5ccccc5n4-c4ccccc4)cc(N(c4ccc(-c5ccccc5)cc4)c4cccc5c4oc4ccccc45)c3)c3cccc4c3oc3ccccc34)cc2)cc1] | 64% |
| Com 36-27 | 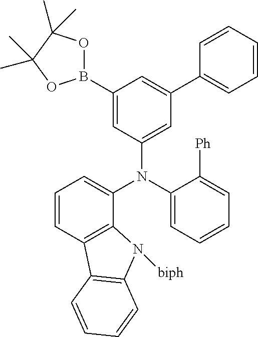 | 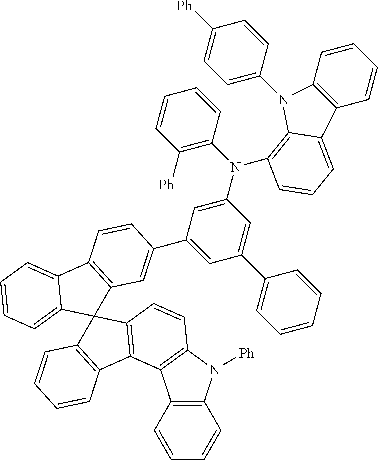 [SMILES: c1ccc(-c2ccc(-n3c4ccccc4c4cccc(N(c5cc(-c6ccccc6)cc(-c6ccc7c(c6)C6(c8ccccc8-7)c7ccccc7-c7c6ccc6c7c7ccccc7n6-c6ccccc6)c5)c5ccccc5-c5ccccc5)c43)cc2)cc1] | 54% |
| Com 36-28 | 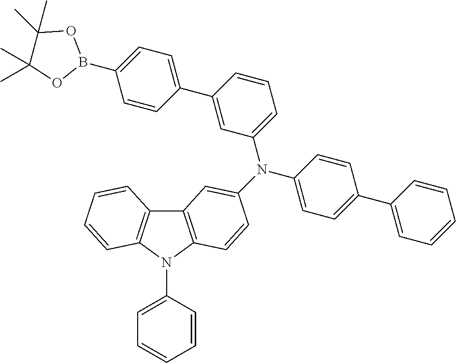 [SMILES: CC1(C)OB(c2ccc(-c3cccc(N(c4ccc(-c5ccccc5)cc4)c4ccc5c(c4)c4ccccc4n5-c4ccccc4)c3)cc2)OC1(C)C] | 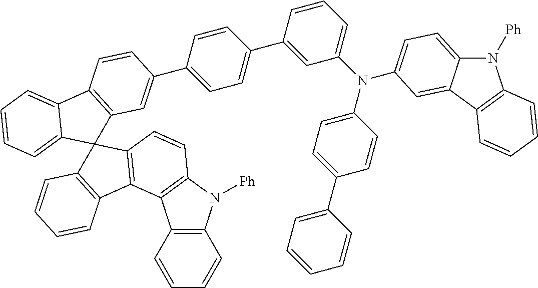 [SMILES: c1ccc(-c2ccc(N(c3cccc(-c4ccc(-c5ccc6c(c5)C5(c7ccccc7-6)c6ccccc6-c6c5ccc5c6c6ccccc6n5-c5ccccc5)cc4)c3)c3ccc4c(c3)c3ccccc3n4-c3ccccc3)cc2)cc1] | 65% |
| Com 36-29 | 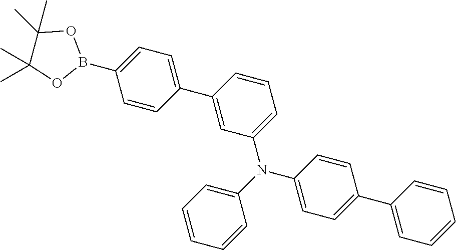 [SMILES: CC1(C)OB(c2ccc(-c3cccc(N(c4ccccc4)c4ccc(-c5ccccc5)cc4)c3)cc2)OC1(C)C] | 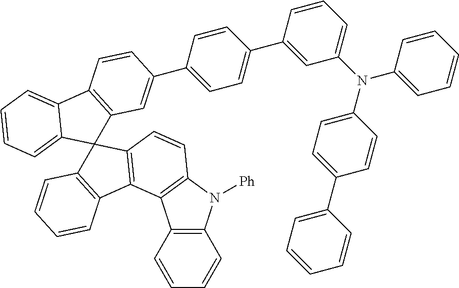 [SMILES: c1ccc(-c2ccc(N(c3ccccc3)c3cccc(-c4ccc(-c5ccc6c(c5)C5(c7ccccc7-6)c6ccccc6-c6c5ccc5c6c6ccccc6n5-c5ccccc5)cc4)c3)cc2)cc1] | 71% |
| Com 36-30 | 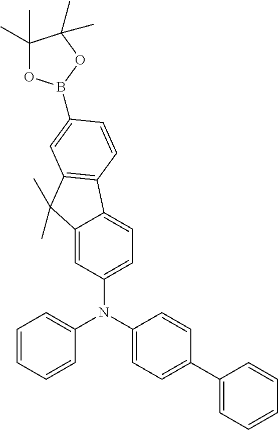 [SMILES: CC1(C)c2cc(B3OC(C)(C)C(C)(C)O3)ccc2-c2ccc(N(c3ccccc3)c3ccc(-c4ccccc4)cc3)cc21] | 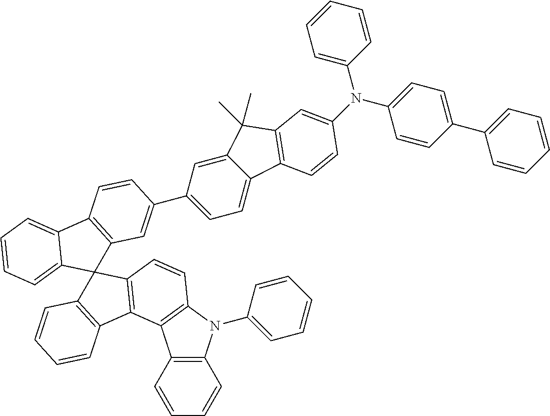 [SMILES: CC1(C)c2cc(-c3ccc4c(c3)C3(c5ccccc5-4)c4ccccc4-c4c3ccc3c4c4ccccc4n3-c3ccccc3)ccc2-c2ccc(N(c3ccccc3)c3ccc(-c4ccccc4)cc3)cc21] | 72% |
| Com 36-31 | 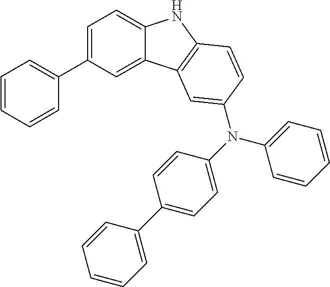 [SMILES: c1ccc(-c2ccc(N(c3ccccc3)c3ccc4[nH]c5ccc(-c6ccccc6)cc5c4c3)cc2)cc1] | 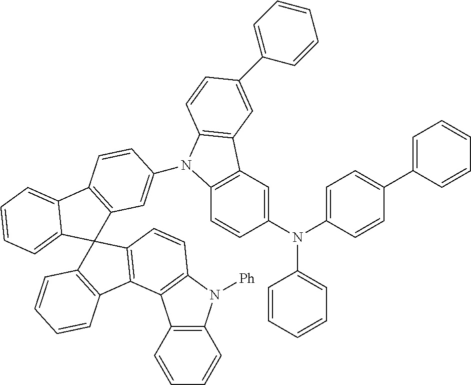 [SMILES: c1ccc(-c2ccc(N(c3ccccc3)c3ccc4c(c3)c3cc(-c5ccccc5)ccc3n4-c3ccc4c(c3)C3(c5ccccc5-4)c4ccccc4-c4c3ccc3c4c4ccccc4n3-c3ccccc3)cc2)cc1] | 62% |
| Com 36-32 | 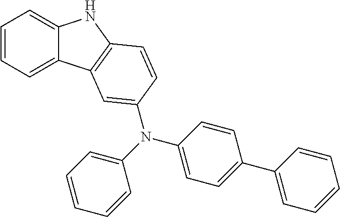 [SMILES: c1ccc(-c2ccc(N(c3ccccc3)c3ccc4[nH]c5ccccc5c4c3)cc2)cc1] | 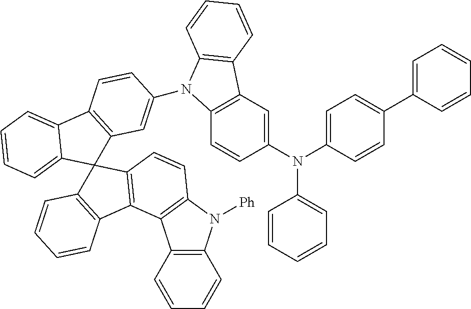 [SMILES: c1ccc(-c2ccc(N(c3ccccc3)c3ccc4c(c3)c3ccccc3n4-c3ccc4c(c3)C3(c5ccccc5-4)c4ccccc4-c4c3ccc3c4c4ccccc4n3-c3ccccc3)cc2)cc1] | 67% |
| Com 36-33 | 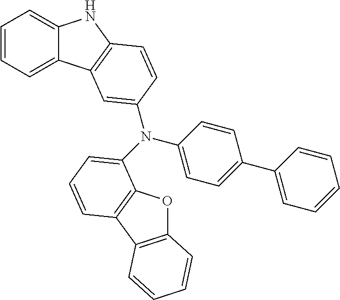 [SMILES: c1ccc(-c2ccc(N(c3ccc4[nH]c5ccccc5c4c3)c3cccc4c3oc3ccccc34)cc2)cc1] | 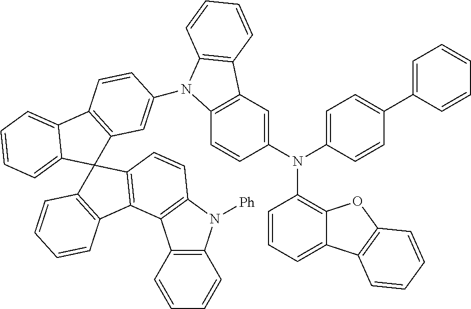 [SMILES: c1ccc(-c2ccc(N(c3ccc4c(c3)c3ccccc3n4-c3ccc4c(c3)C3(c5ccccc5-4)c4ccccc4-c4c3ccc3c4c4ccccc4n3-c3ccccc3)c3cccc4c3oc3ccccc34)cc2)cc1] | 69% |
| Com 36-34 | 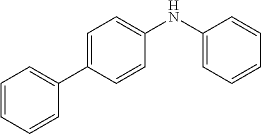 [SMILES: c1ccc(Nc2ccc(-c3ccccc3)cc2)cc1] | 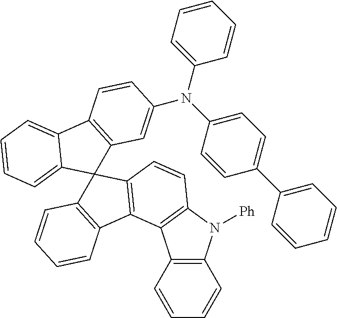 [SMILES: c1ccc(-c2ccc(N(c3ccccc3)c3ccc4c(c3)C3(c5ccccc5-4)c4ccccc4-c4c3ccc3c4c4ccccc4n3-c3ccccc3)cc2)cc1] | 65% |
| Com 36-35 | 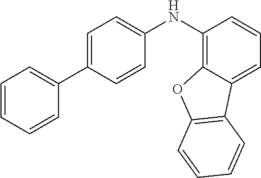 [SMILES: c1ccc(-c2ccc(Nc3cccc4c3oc3ccccc34)cc2)cc1] | 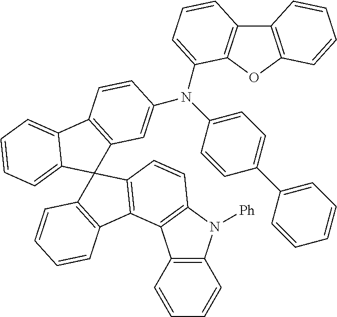 [SMILES: c1ccc(-c2ccc(N(c3ccc4c(c3)C3(c5ccccc5-4)c4ccccc4-c4c3ccc3c4c4ccccc4n3-c3ccccc3)c3cccc4c3oc3ccccc34)cc2)cc1] | 70% |
| Com 36-36 | 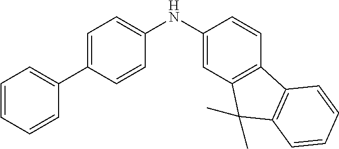 [SMILES: CC1(C)c2ccccc2-c2ccc(Nc3ccc(-c4ccccc4)cc3)cc21] | 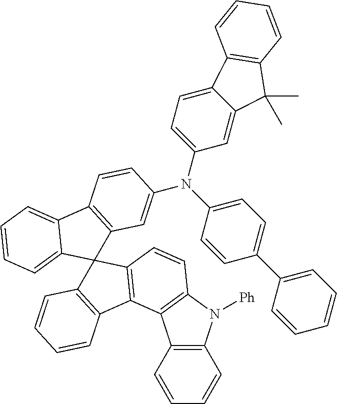 [SMILES: CC1(C)c2ccccc2-c2ccc(N(c3ccc(-c4ccccc4)cc3)c3ccc4c(c3)C3(c5ccccc5-4)c4ccccc4-c4c3ccc3c4c4ccccc4n3-c3ccccc3)cc21] | 75% |
| Com 36-37 | 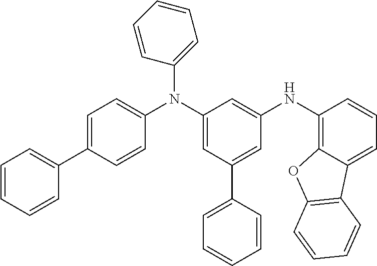 [SMILES: c1ccc(-c2ccc(N(c3ccccc3)c3cc(Nc4cccc5c4oc4ccccc45)cc(-c4ccccc4)c3)cc2)cc1] | 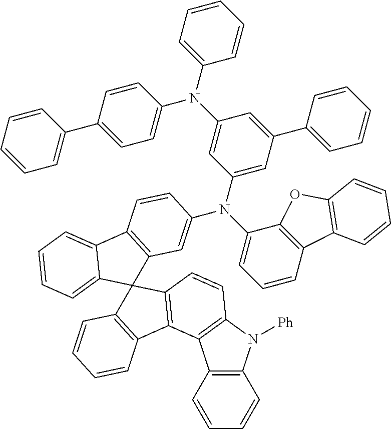 [SMILES: c1ccc(-c2ccc(N(c3ccccc3)c3cc(-c4ccccc4)cc(N(c4ccc5c(c4)C4(c6ccccc6-5)c5ccccc5-c5c4ccc4c5c5ccccc5n4-c4ccccc4)c4cccc5c4oc4ccccc45)c3)cc2)cc1] | 71% |
| Com 36-38 | 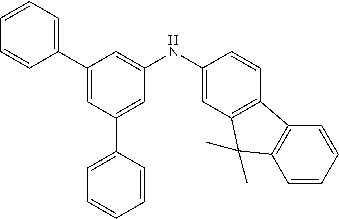 [SMILES: CC1(C)c2ccccc2-c2ccc(Nc3cc(-c4ccccc4)cc(-c4ccccc4)c3)cc21] | 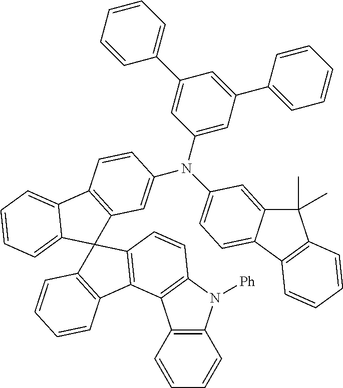 [SMILES: CC1(C)c2ccccc2-c2ccc(N(c3cc(-c4ccccc4)cc(-c4ccccc4)c3)c3ccc4c(c3)C3(c5ccccc5-4)c4ccccc4-c4c3ccc3c4c4ccccc4n3-c3ccccc3)cc21] | 84% |
| Com 36-39 | 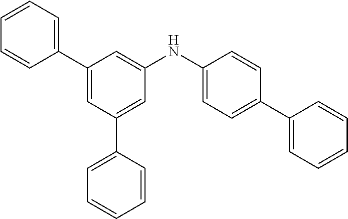 [SMILES: c1ccc(-c2ccc(Nc3cc(-c4ccccc4)cc(-c4ccccc4)c3)cc2)cc1] | 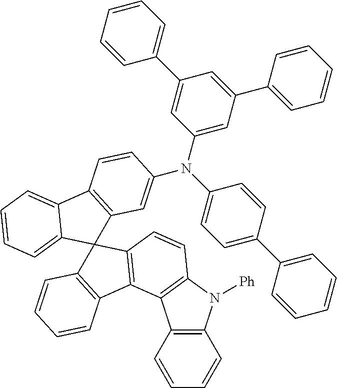 [SMILES: c1ccc(-c2ccc(N(c3cc(-c4ccccc4)cc(-c4ccccc4)c3)c3ccc4c(c3)C3(c5ccccc5-4)c4ccccc4-c4c3ccc3c4c4ccccc4n3-c3ccccc3)cc2)cc1] | 78% |
| Com 36-40 | 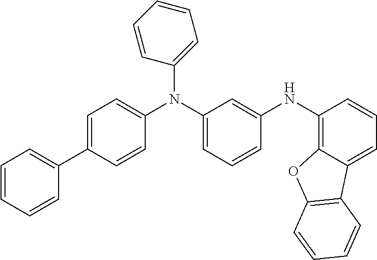 [SMILES: c1ccc(-c2ccc(N(c3ccccc3)c3cccc(Nc4cccc5c4oc4ccccc45)c3)cc2)cc1] | 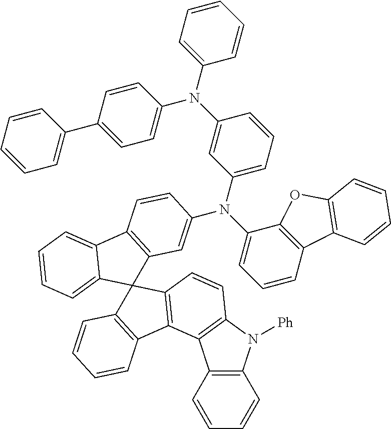 [SMILES: c1ccc(-c2ccc(N(c3ccccc3)c3cccc(N(c4ccc5c(c4)C4(c6ccccc6-5)c5ccccc5-c5c4ccc4c5c5ccccc5n4-c4ccccc4)c4cccc5c4oc4ccccc45)c3)cc2)cc1] | 81% |
| Com 36-41 | 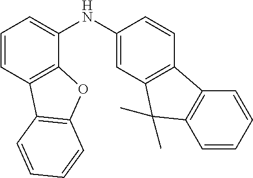 [SMILES: CC1(C)c2ccccc2-c2ccc(Nc3cccc4c3oc3ccccc34)cc21] | 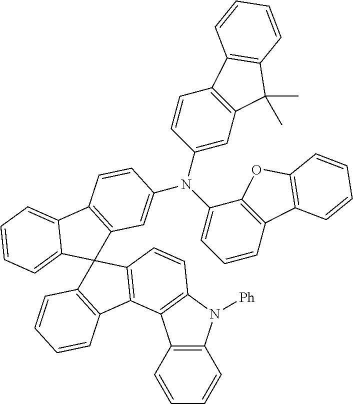 [SMILES: CC1(C)c2ccccc2-c2ccc(N(c3ccc4c(c3)C3(c5ccccc5-4)c4ccccc4-c4c3ccc3c4c4ccccc4n3-c3ccccc3)c3cccc4c3oc3ccccc34)cc21] | 74% |
| Com 36-42 | 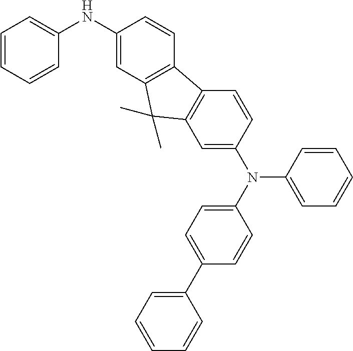 [SMILES: CC1(C)c2cc(Nc3ccccc3)ccc2-c2ccc(N(c3ccccc3)c3ccc(-c4ccccc4)cc3)cc21] | 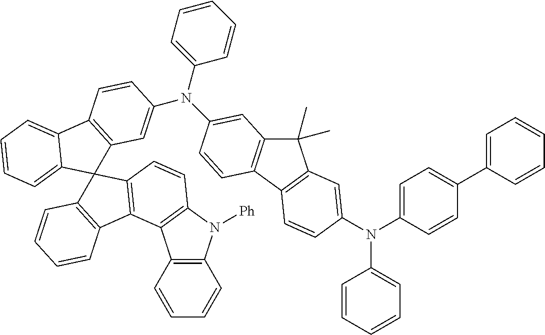 [SMILES: CC1(C)c2cc(N(c3ccccc3)c3ccc(-c4ccccc4)cc3)ccc2-c2ccc(N(c3ccccc3)c3ccc4c(c3)C3(c5ccccc5-4)c4ccccc4-c4c3ccc3c4c4ccccc4n3-c3ccccc3)cc21] | 74% |
| Com 36-43 | 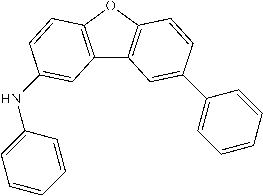 [SMILES: c1ccc(Nc2ccc3oc4ccc(-c5ccccc5)cc4c3c2)cc1] | 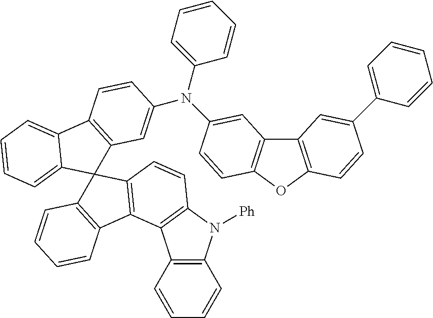 [SMILES: c1ccc(-c2ccc3oc4ccc(N(c5ccccc5)c5ccc6c(c5)C5(c7ccccc7-6)c6ccccc6-c6c5ccc5c6c6ccccc6n5-c5ccccc5)cc4c3c2)cc1] | 76% |
| Com 36-44 | 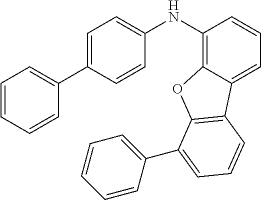 [SMILES: c1ccc(-c2ccc(Nc3cccc4c3oc3c(-c5ccccc5)cccc34)cc2)cc1] | 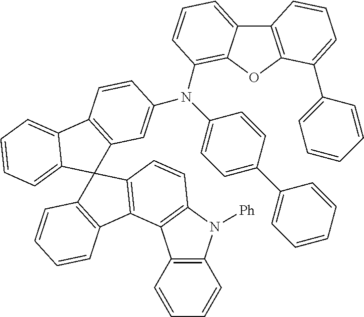 [SMILES: c1ccc(-c2ccc(N(c3ccc4c(c3)C3(c5ccccc5-4)c4ccccc4-c4c3ccc3c4c4ccccc4n3-c3ccccc3)c3cccc4c3oc3c(-c5ccccc5)cccc34)cc2)cc1] | 71% |
| Com 36-45 | 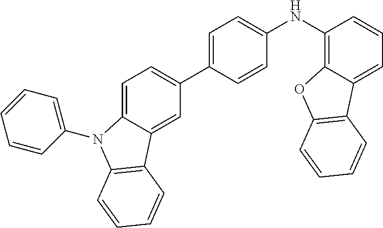 [SMILES: c1ccc(-n2c3ccccc3c3cc(-c4ccc(Nc5cccc6c5oc5ccccc56)cc4)ccc32)cc1] | 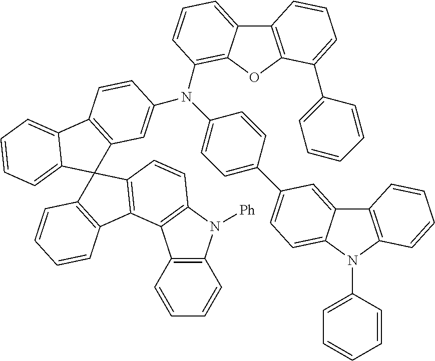 [SMILES: c1ccc(-c2cccc3c2oc2c(N(c4ccc(-c5ccc6c(c5)c5ccccc5n6-c5ccccc5)cc4)c4ccc5c(c4)C4(c6ccccc6-5)c5ccccc5-c5c4ccc4c5c5ccccc5n4-c4ccccc4)cccc23)cc1] | 77% |
| Com 36-46 | 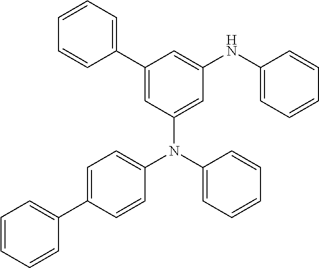 [SMILES: c1ccc(Nc2cc(-c3ccccc3)cc(N(c3ccccc3)c3ccc(-c4ccccc4)cc3)c2)cc1] | 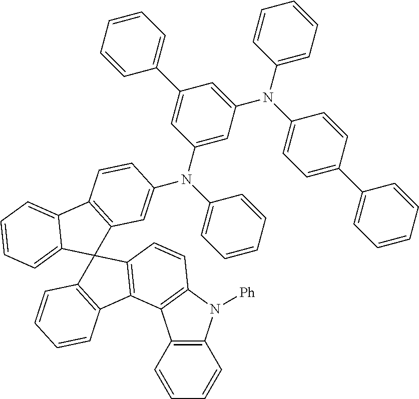 [SMILES: c1ccc(-c2ccc(N(c3ccccc3)c3cc(-c4ccccc4)cc(N(c4ccccc4)c4ccc5c(c4)C4(c6ccccc6-5)c5ccccc5-c5c4ccc4c5c5ccccc5n4-c4ccccc4)c3)cc2)cc1] | 79% |
| Com 36-47 | 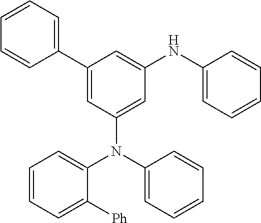 [SMILES: c1ccc(Nc2cc(-c3ccccc3)cc(N(c3ccccc3)c3ccccc3-c3ccccc3)c2)cc1] | 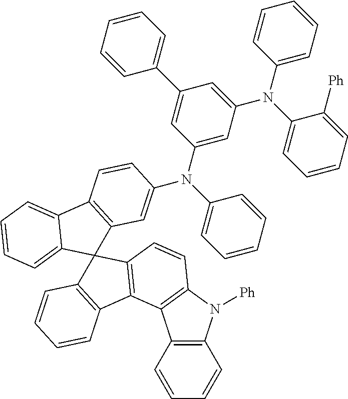 [SMILES: c1ccc(-c2cc(N(c3ccccc3)c3ccc4c(c3)C3(c5ccccc5-4)c4ccccc4-c4c3ccc3c4c4ccccc4n3-c3ccccc3)cc(N(c3ccccc3)c3ccccc3-c3ccccc3)c2)cc1] | 74% |
| Com 36-48 | 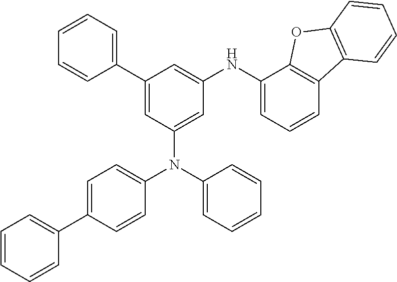 [SMILES: c1ccc(-c2ccc(N(c3ccccc3)c3cc(Nc4cccc5c4oc4ccccc45)cc(-c4ccccc4)c3)cc2)cc1] | 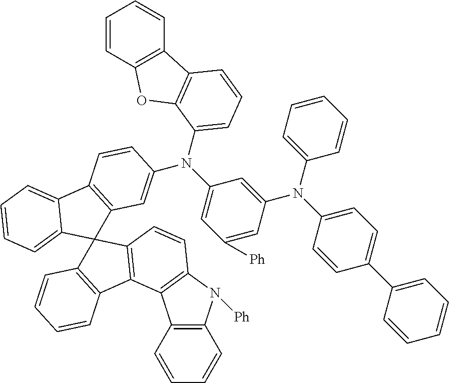 [SMILES: c1ccc(-c2ccc(N(c3ccccc3)c3cc(-c4ccccc4)cc(N(c4ccc5c(c4)C4(c6ccccc6-5)c5ccccc5-c5c4ccc4c5c5ccccc5n4-c4ccccc4)c4cccc5c4oc4ccccc45)c3)cc2)cc1] | 75% |
| Com 36-49 | 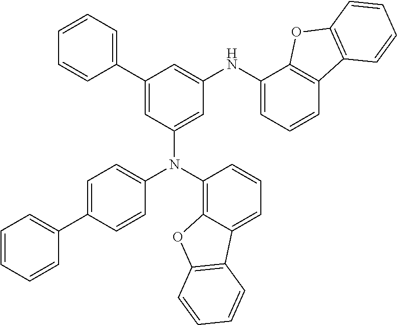 [SMILES: c1ccc(-c2ccc(N(c3cc(Nc4cccc5c4oc4ccccc45)cc(-c4ccccc4)c3)c3cccc4c3oc3ccccc34)cc2)cc1] | 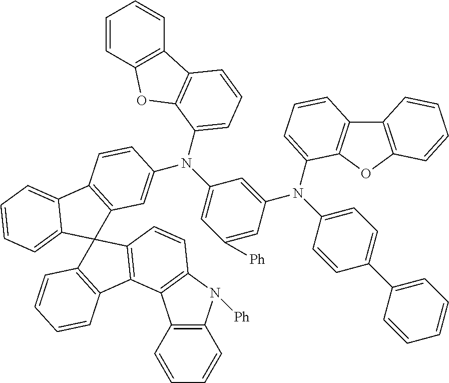 [SMILES: c1ccc(-c2ccc(N(c3cc(-c4ccccc4)cc(N(c4ccc5c(c4)C4(c6ccccc6-5)c5ccccc5-c5c4ccc4c5c5ccccc5n4-c4ccccc4)c4cccc5c4oc4ccccc45)c3)c3cccc4c3oc3ccccc34)cc2)cc1] | 68% |
| Com 36-50 | 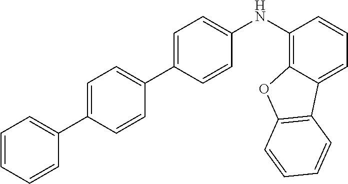 [SMILES: c1ccc(-c2ccc(-c3ccc(Nc4cccc5c4oc4ccccc45)cc3)cc2)cc1] | 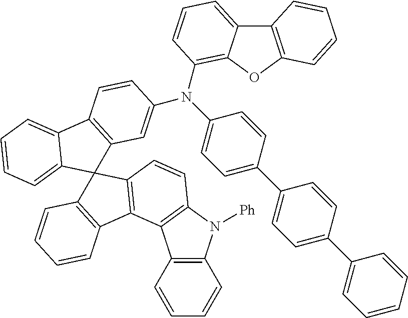 [SMILES: c1ccc(-c2ccc(-c3ccc(N(c4ccc5c(c4)C4(c6ccccc6-5)c5ccccc5-c5c4ccc4c5c5ccccc5n4-c4ccccc4)c4cccc5c4oc4ccccc45)cc3)cc2)cc1] | 74% |
| Com 36-51 | 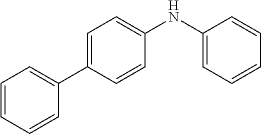 [SMILES: c1ccc(Nc2ccc(-c3ccccc3)cc2)cc1] | 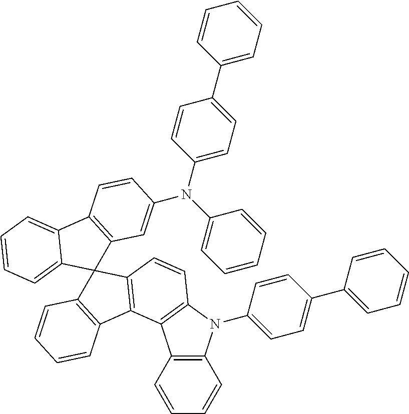 [SMILES: c1ccc(-c2ccc(N(c3ccccc3)c3ccc4c(c3)C3(c5ccccc5-4)c4ccccc4-c4c3ccc3c4c4ccccc4n3-c3ccc(-c4ccccc4)cc3)cc2)cc1] | 63% |
| Com 36-52 | 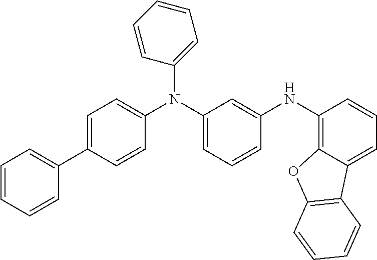 [SMILES: c1ccc(-c2ccc(N(c3ccccc3)c3cccc(Nc4cccc5c4oc4ccccc45)c3)cc2)cc1] | 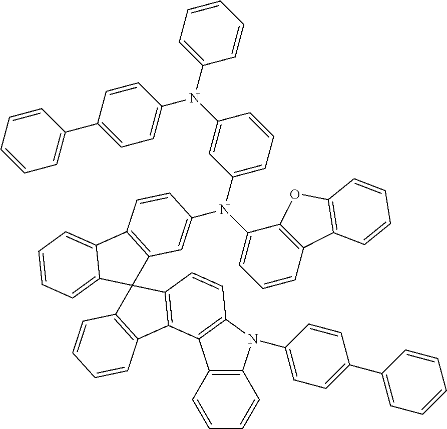 [SMILES: c1ccc(-c2ccc(N(c3ccccc3)c3cccc(N(c4ccc5c(c4)C4(c6ccccc6-5)c5ccccc5-c5c4ccc4c5c5ccccc5n4-c4ccc(-c5ccccc5)cc4)c4cccc5c4oc4ccccc45)c3)cc2)cc1] | 65% |
| Com 36-53 | 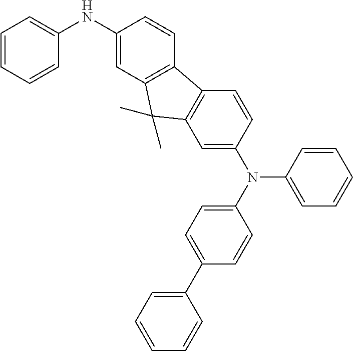 [SMILES: CC1(C)c2cc(Nc3ccccc3)ccc2-c2ccc(N(c3ccccc3)c3ccc(-c4ccccc4)cc3)cc21] | 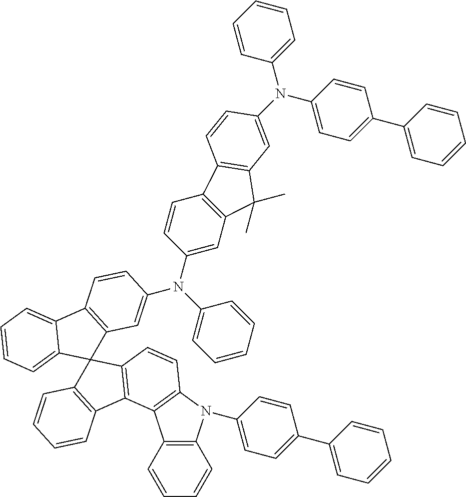 [SMILES: CC1(C)c2cc(N(c3ccccc3)c3ccc(-c4ccccc4)cc3)ccc2-c2ccc(N(c3ccccc3)c3ccc4c(c3)C3(c5ccccc5-4)c4ccccc4-c4c3ccc3c4c4ccccc4n3-c3ccc(-c4ccccc4)cc3)cc21] | 71% |
| Com 36-54 | 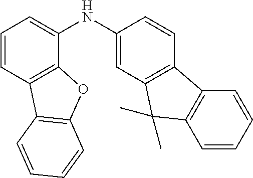 [SMILES: CC1(C)c2ccccc2-c2ccc(Nc3cccc4c3oc3ccccc34)cc21] | 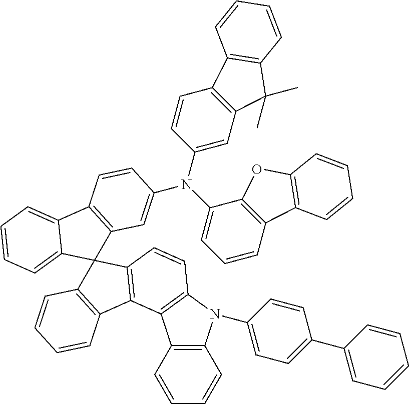 [SMILES: CC1(C)c2ccccc2-c2ccc(N(c3ccc4c(c3)C3(c5ccccc5-4)c4ccccc4-c4c3ccc3c4c4ccccc4n3-c3ccc(-c4ccccc4)cc3)c3cccc4c3oc3ccccc34)cc21] | 60% |
| Com 36-55 | 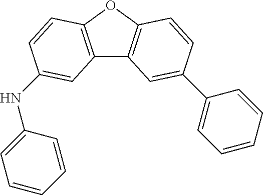 [SMILES: c1ccc(Nc2ccc3oc4ccc(-c5ccccc5)cc4c3c2)cc1] | 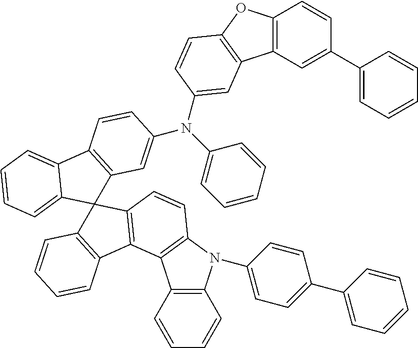 [SMILES: c1ccc(-c2ccc(-n3c4ccccc4c4c5c(ccc43)C3(c4ccccc4-c4ccc(N(c6ccccc6)c6ccc7oc8ccc(-c9ccccc9)cc8c7c6)cc43)c3ccccc3-5)cc2)cc1] | 71% |
| Com 36-56 | 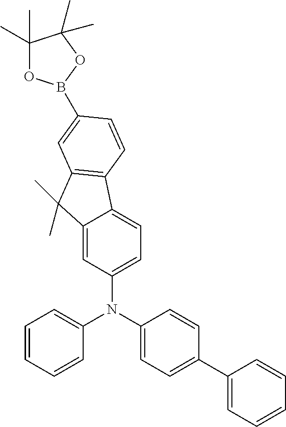 [SMILES: CC1(C)c2cc(B3OC(C)(C)C(C)(C)O3)ccc2-c2ccc(N(c3ccccc3)c3ccc(-c4ccccc4)cc3)cc21] | 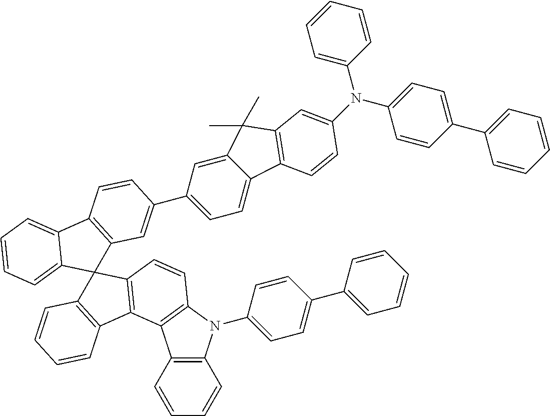 [SMILES: CC1(C)c2cc(-c3ccc4c(c3)C3(c5ccccc5-4)c4ccccc4-c4c3ccc3c4c4ccccc4n3-c3ccc(-c4ccccc4)cc3)ccc2-c2ccc(N(c3ccccc3)c3ccc(-c4ccccc4)cc3)cc21] | 70% |
| Com 36-57 | 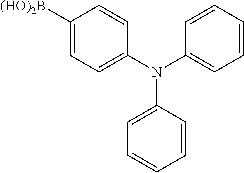 [SMILES: OB(O)c1ccc(N(c2ccccc2)c2ccccc2)cc1] | 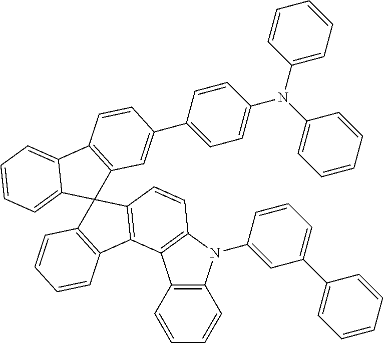 [SMILES: c1ccc(-c2cccc(-n3c4ccccc4c4c5c(ccc43)C3(c4ccccc4-c4ccc(-c6ccc(N(c7ccccc7)c7ccccc7)cc6)cc43)c3ccccc3-5)c2)cc1] | 73% |
| Com 36-58 | 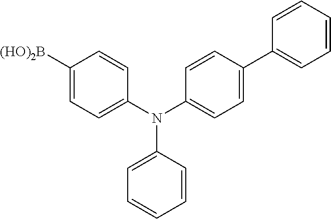 [SMILES: OB(O)c1ccc(N(c2ccccc2)c2ccc(-c3ccccc3)cc2)cc1] | 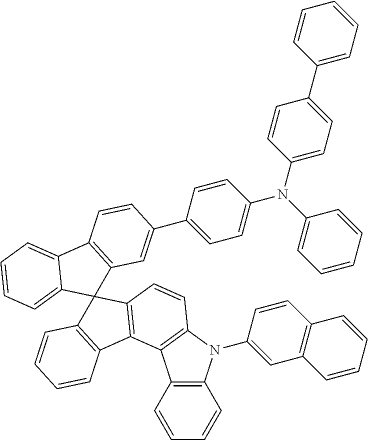 [SMILES: c1ccc(-c2ccc(N(c3ccccc3)c3ccc(-c4ccc5c(c4)C4(c6ccccc6-5)c5ccccc5-c5c4ccc4c5c5ccccc5n4-c4ccc5ccccc5c4)cc3)cc2)cc1] | 75% |
| Com 36-59 | 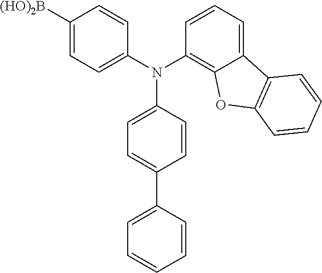 [SMILES: OB(O)c1ccc(N(c2ccc(-c3ccccc3)cc2)c2cccc3c2oc2ccccc23)cc1] | 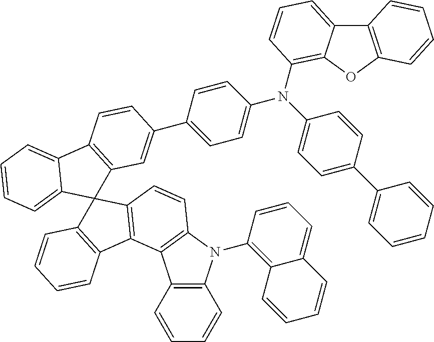 [SMILES: c1ccc(-c2ccc(N(c3ccc(-c4ccc5c(c4)C4(c6ccccc6-5)c5ccccc5-c5c4ccc4c5c5ccccc5n4-c4cccc5ccccc45)cc3)c3cccc4c3oc3ccccc34)cc2)cc1] | 72% |

### [Preparation Example 7] Preparation of Compound G-1

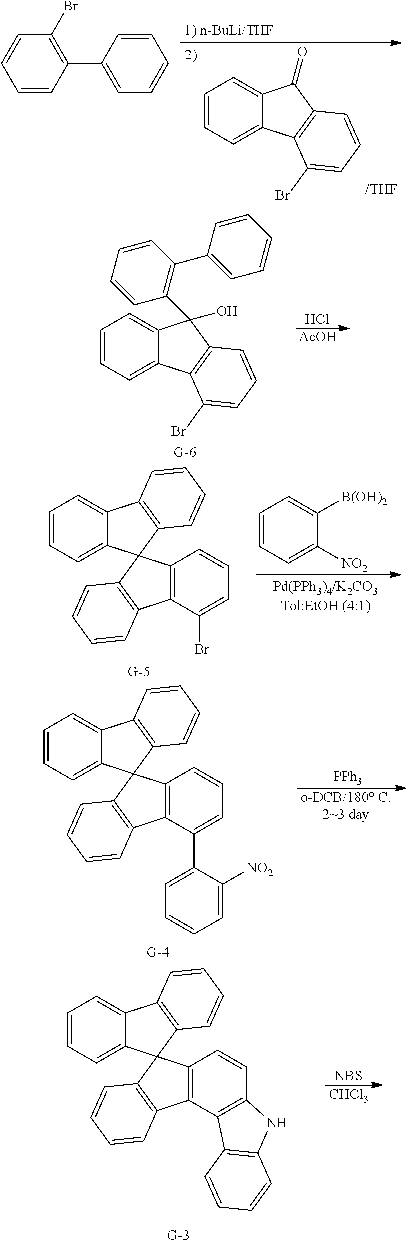

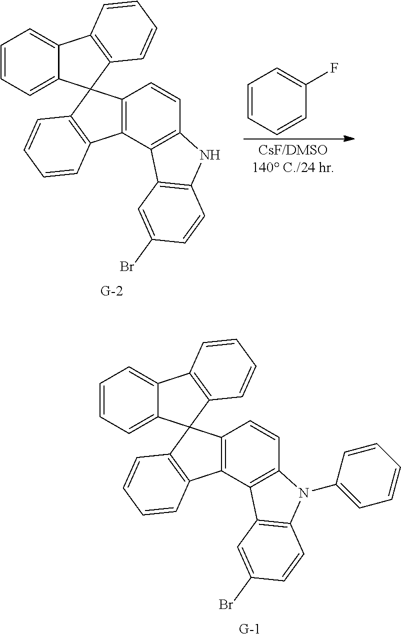

Preparation of Compound G-3

A compound G-3 was prepared in the same manner as that of Preparation Example 6, except that 2-bromo-1,1′-biphenyl (38.00 mmol) was used instead of 2-bromo-4′-methoxy-1,1′-biphenyl as a starting material.

Preparation of Compound G-2

10 g (24.66 mmol) of the compound G-3 was dissolved in 110 mL of chloroform, 4.83 g (27.13 mmol) of N-bromosuccinimide was added, and stirring was performed at 40° C. for 24 hours. After the reaction was completed, a sodium thiosulfate (Na₂S₂O₃) aqueous solution was added to stop the reaction, extraction was performed using MC and H₂O, the solvent was removed, and then purification was performed by column chromatography with Hex and MC, thereby obtaining 8.48 g (71%) of a target compound G-2.

Preparation of Compound G-1

10 g (20.64 mmol) of P-3 was dissolved in 120 mL of DMSO, 2.38 g (24.77 mmol) of fluorobenzene and 4.19 g (27.55 mmol) of CsF were added thereto, and stirring was performed under reflux at 140° C. for 24 hours. After the reaction was completed, the reaction solution was cooled to room temperature, the produced solid was filtered and then washed with water, ethanol, Hex, and EA, and then recrystallization was performed with Hex/EA, thereby obtaining 9.02 g (71%) of a target compound G-1.

### [Example 7] Preparation of Compounds P13 and P14

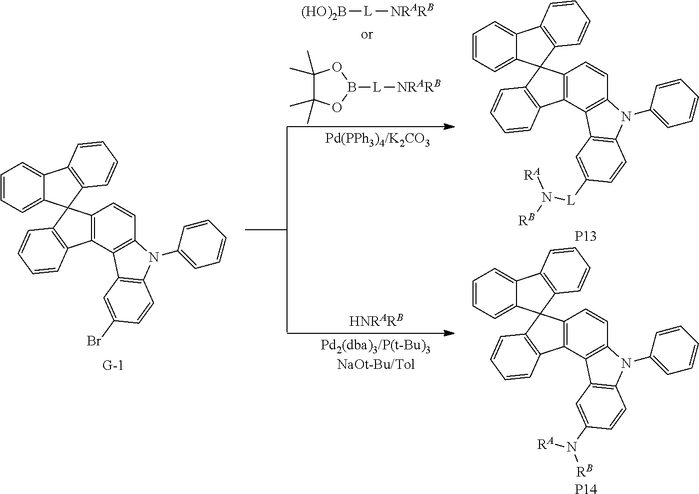

Preparation of Compound P13

A target compound P13 was obtained through a Suzuki coupling reaction using the compound G-1, a substituted boronic acid or boronic ester compound, and a palladium catalyst.

Preparation of Compound P14

A target compound P14 was obtained through palladium-amination using the compound G-1, a secondary amine compound, and a palladium catalyst.

The structures of the substituted boronic acid, boronic ester, and secondary amine compound as the reactants and the structures and yields of the prepared compounds P13 and P14 are shown in Table 7.

TABLE 7

| Comp | | | |
|---|---|---|---|
| ound | | | |
| No. | Reactant | Product (P13, P14) | Yield |
| Com 37-1 | 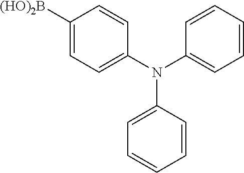 [SMILES: OB(O)c1ccc(N(c2ccccc2)c2ccccc2)cc1] | 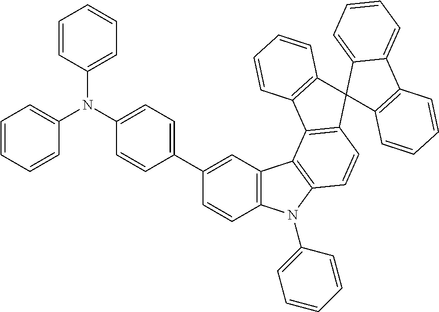 [SMILES: c1ccc(N(c2ccccc2)c2ccc(-c3ccc4c(c3)c3c5c(ccc3n4-c3ccccc3)C3(c4ccccc4-c4ccccc43)c3ccccc3-5)cc2)cc1] | 74% |
| Com 37-2 | 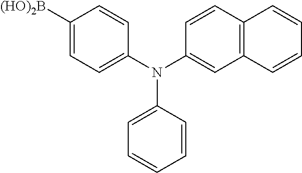 [SMILES: OB(O)c1ccc(N(c2ccccc2)c2ccc3ccccc3c2)cc1] | 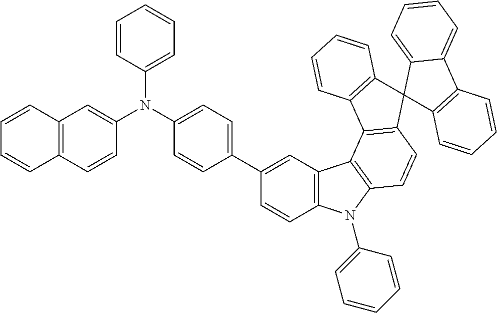 [SMILES: c1ccc(N(c2ccc(-c3ccc4c(c3)c3c5c(ccc3n4-c3ccccc3)C3(c4ccccc4-c4ccccc43)c3ccccc3-5)cc2)c2ccc3ccccc3c2)cc1] | 79% |
| Com 37-3 | 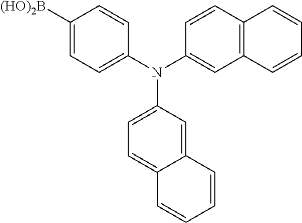 [SMILES: OB(O)c1ccc(N(c2ccc3ccccc3c2)c2ccc3ccccc3c2)cc1] | 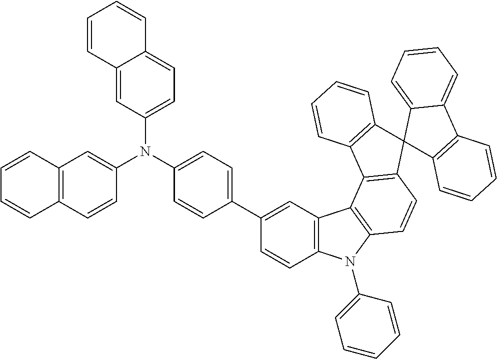 [SMILES: c1ccc(-n2c3ccc(-c4ccc(N(c5ccc6ccccc6c5)c5ccc6ccccc6c5)cc4)cc3c3c4c(ccc32)C2(c3ccccc3-c3ccccc32)c2ccccc2-4)cc1] | 77% |
| Com 37-4 | 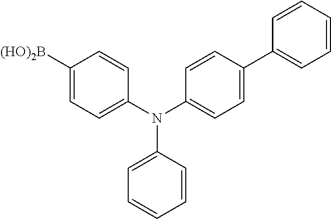 [SMILES: OB(O)c1ccc(N(c2ccccc2)c2ccc(-c3ccccc3)cc2)cc1] | 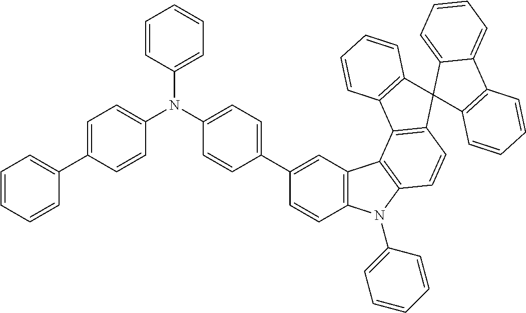 [SMILES: c1ccc(-c2ccc(N(c3ccccc3)c3ccc(-c4ccc5c(c4)c4c6c(ccc4n5-c4ccccc4)C4(c5ccccc5-c5ccccc54)c4ccccc4-6)cc3)cc2)cc1] | 72% |
| Com 37-5 | 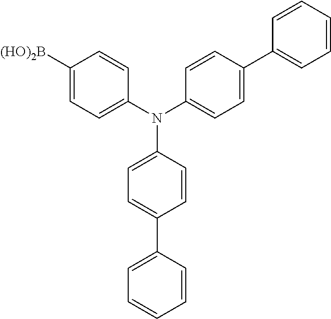 [SMILES: OB(O)c1ccc(N(c2ccc(-c3ccccc3)cc2)c2ccc(-c3ccccc3)cc2)cc1] | 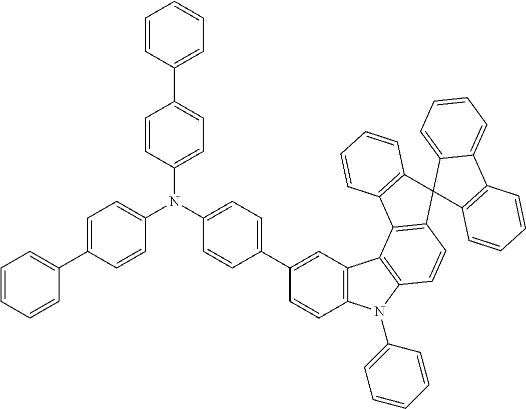 [SMILES: c1ccc(-c2ccc(N(c3ccc(-c4ccccc4)cc3)c3ccc(-c4ccc5c(c4)c4c6c(ccc4n5-c4ccccc4)C4(c5ccccc5-c5ccccc54)c4ccccc4-6)cc3)cc2)cc1] | 81% |
| Com 37-6 | 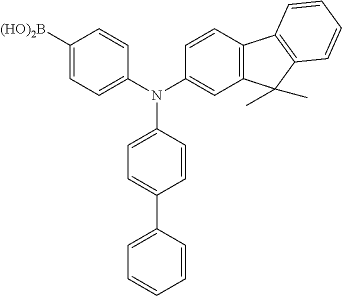 [SMILES: CC1(C)c2ccccc2-c2ccc(N(c3ccc(B(O)O)cc3)c3ccc(-c4ccccc4)cc3)cc21] | 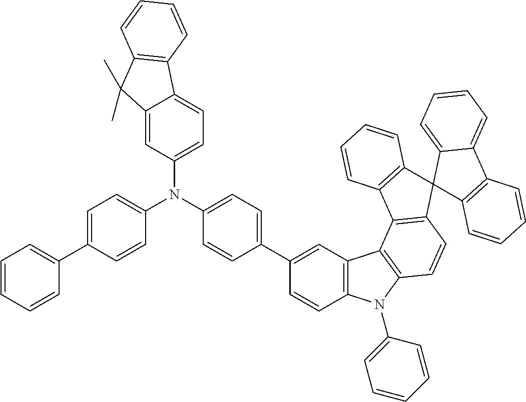 [SMILES: CC1(C)c2ccccc2-c2ccc(N(c3ccc(-c4ccccc4)cc3)c3ccc(-c4ccc5c(c4)c4c6c(ccc4n5-c4ccccc4)C4(c5ccccc5-c5ccccc54)c4ccccc4-6)cc3)cc21] | 80% |
| Com 37-7 | 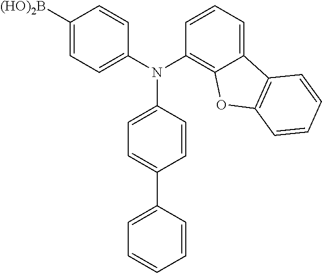 [SMILES: OB(O)c1ccc(N(c2ccc(-c3ccccc3)cc2)c2cccc3c2oc2ccccc23)cc1] | 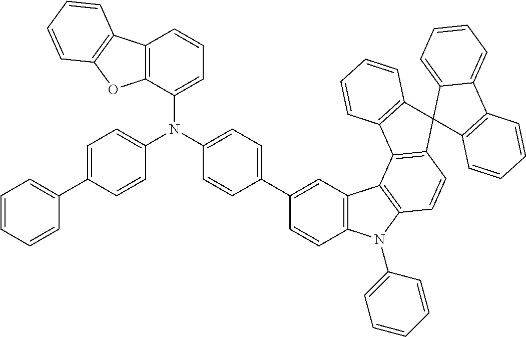 [SMILES: c1ccc(-c2ccc(N(c3ccc(-c4ccc5c(c4)c4c6c(ccc4n5-c4ccccc4)C4(c5ccccc5-c5ccccc54)c4ccccc4-6)cc3)c3cccc4c3oc3ccccc34)cc2)cc1] | 74% |
| Com 37-8 | 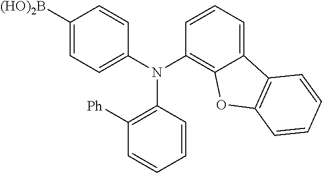 [SMILES: OB(O)c1ccc(N(c2ccccc2-c2ccccc2)c2cccc3c2oc2ccccc23)cc1] | 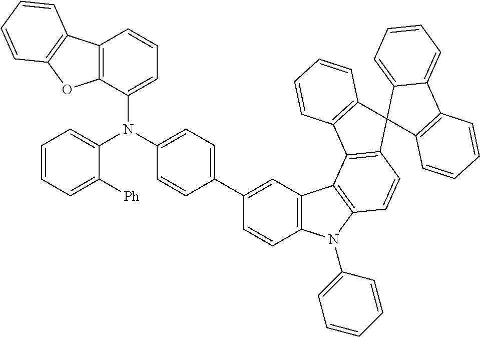 [SMILES: c1ccc(-c2ccccc2N(c2ccc(-c3ccc4c(c3)c3c5c(ccc3n4-c3ccccc3)C3(c4ccccc4-c4ccccc43)c3ccccc3-5)cc2)c2cccc3c2oc2ccccc23)cc1] | 60% |
| Com 37-9 | 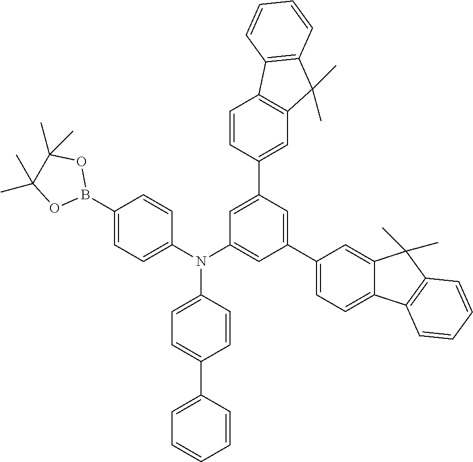 [SMILES: CC1(C)c2ccccc2-c2ccc(-c3cc(-c4ccc5c(c4)C(C)(C)c4ccccc4-5)cc(N(c4ccc(B5OC(C)(C)C(C)(C)O5)cc4)c4ccc(-c5ccccc5)cc4)c3)cc21] | 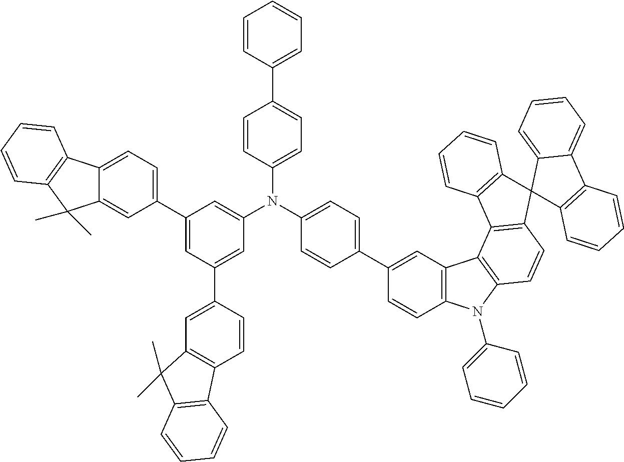 [SMILES: CC1(C)c2ccccc2-c2ccc(-c3cc(-c4ccc5c(c4)C(C)(C)c4ccccc4-5)cc(N(c4ccc(-c5ccccc5)cc4)c4ccc(-c5ccc6c(c5)c5c7c(ccc5n6-c5ccccc5)C5(c6ccccc6-c6ccccc65)c5ccccc5-7)cc4)c3)cc21] | 52% |
| Com 37-10 | 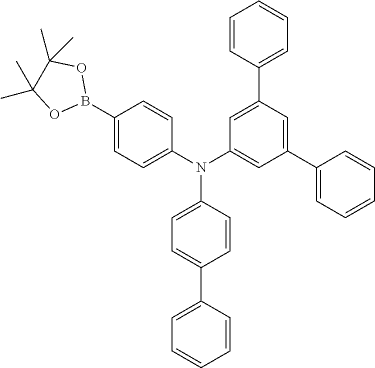 [SMILES: CC1(C)OB(c2ccc(N(c3ccc(-c4ccccc4)cc3)c3cc(-c4ccccc4)cc(-c4ccccc4)c3)cc2)OC1(C)C] | 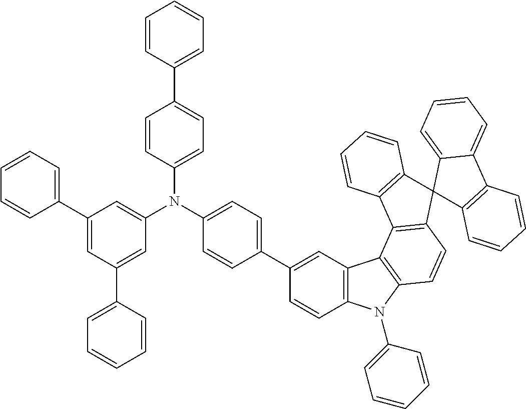 [SMILES: c1ccc(-c2ccc(N(c3ccc(-c4ccc5c(c4)c4c6c(ccc4n5-c4ccccc4)C4(c5ccccc5-c5ccccc54)c4ccccc4-6)cc3)c3cc(-c4ccccc4)cc(-c4ccccc4)c3)cc2)cc1] | 70% |
| Com 37-11 | 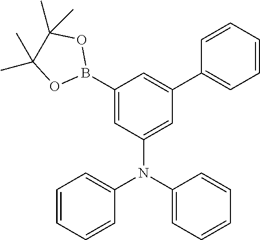 [SMILES: CC1(C)OB(c2cc(-c3ccccc3)cc(N(c3ccccc3)c3ccccc3)c2)OC1(C)C] | 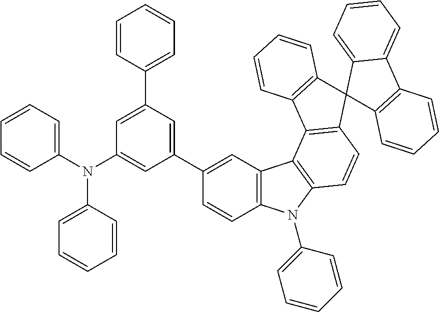 [SMILES: c1ccc(-c2cc(-c3ccc4c(c3)c3c5c(ccc3n4-c3ccccc3)C3(c4ccccc4-c4ccccc43)c3ccccc3-5)cc(N(c3ccccc3)c3ccccc3)c2)cc1] | 65% |
| Com 37-12 | 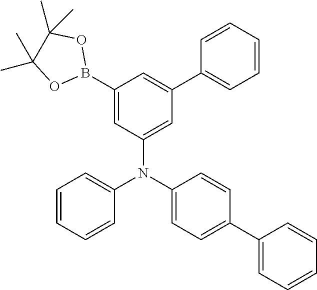 [SMILES: CC1(C)OB(c2cc(-c3ccccc3)cc(N(c3ccccc3)c3ccc(-c4ccccc4)cc3)c2)OC1(C)C] | 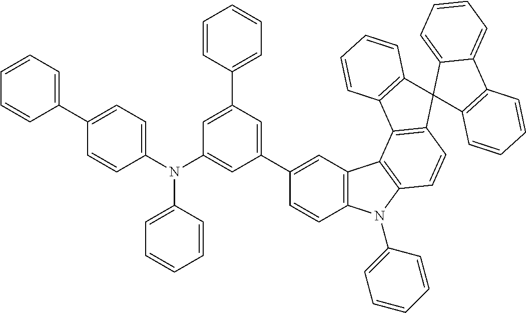 [SMILES: c1ccc(-c2ccc(N(c3ccccc3)c3cc(-c4ccccc4)cc(-c4ccc5c(c4)c4c6c(ccc4n5-c4ccccc4)C4(c5ccccc5-c5ccccc54)c4ccccc4-6)c3)cc2)cc1] | 75% |
| Com 37-13 | 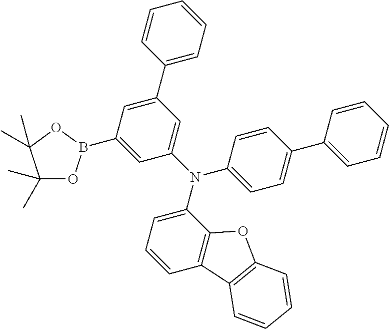 [SMILES: CC1(C)OB(c2cc(-c3ccccc3)cc(N(c3ccc(-c4ccccc4)cc3)c3cccc4c3oc3ccccc34)c2)OC1(C)C] | 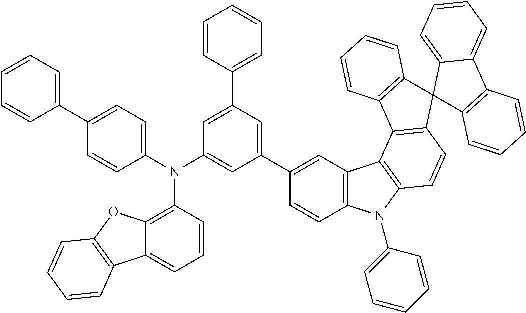 [SMILES: c1ccc(-c2ccc(N(c3cc(-c4ccccc4)cc(-c4ccc5c(c4)c4c6c(ccc4n5-c4ccccc4)C4(c5ccccc5-c5ccccc54)c4ccccc4-6)c3)c3cccc4c3oc3ccccc34)cc2)cc1] | 68% |
| Com 37-14 | 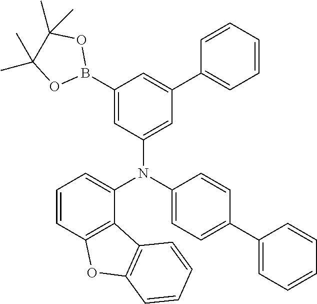 [SMILES: CC1(C)OB(c2cc(-c3ccccc3)cc(N(c3ccc(-c4ccccc4)cc3)c3cccc4oc5ccccc5c34)c2)OC1(C)C] | 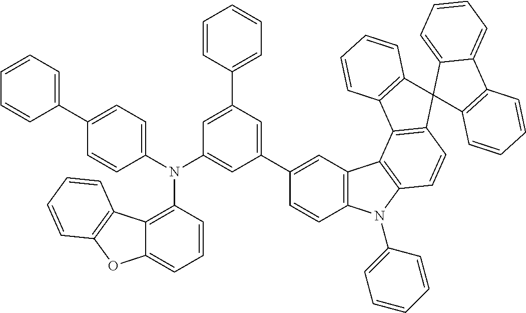 [SMILES: c1ccc(-c2ccc(N(c3cc(-c4ccccc4)cc(-c4ccc5c(c4)c4c6c(ccc4n5-c4ccccc4)C4(c5ccccc5-c5ccccc54)c4ccccc4-6)c3)c3cccc4oc5ccccc5c34)cc2)cc1] | 63% |
| Com 37-15 | 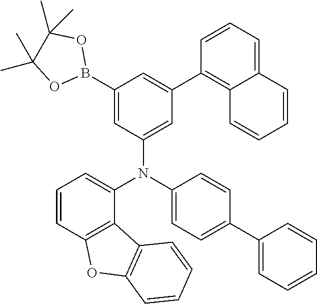 [SMILES: CC1(C)OB(c2cc(-c3cccc4ccccc34)cc(N(c3ccc(-c4ccccc4)cc3)c3cccc4oc5ccccc5c34)c2)OC1(C)C] | 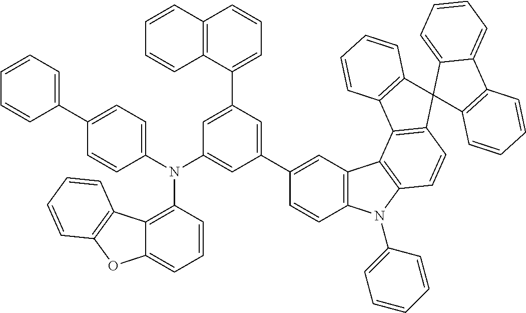 [SMILES: c1ccc(-c2ccc(N(c3cc(-c4ccc5c(c4)c4c6c(ccc4n5-c4ccccc4)C4(c5ccccc5-c5ccccc54)c4ccccc4-6)cc(-c4cccc5ccccc45)c3)c3cccc4oc5ccccc5c34)cc2)cc1] | 59% |
| Com 37-16 | 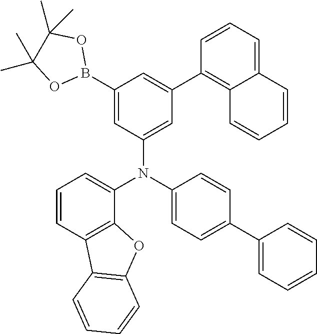 [SMILES: CC1(C)OB(c2cc(-c3cccc4ccccc34)cc(N(c3ccc(-c4ccccc4)cc3)c3cccc4c3oc3ccccc34)c2)OC1(C)C] | 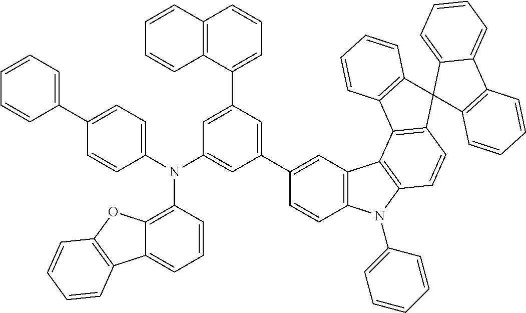 [SMILES: c1ccc(-c2ccc(N(c3cc(-c4ccc5c(c4)c4c6c(ccc4n5-c4ccccc4)C4(c5ccccc5-c5ccccc54)c4ccccc4-6)cc(-c4cccc5ccccc45)c3)c3cccc4c3oc3ccccc34)cc2)cc1] | 75% |
| Com 37-17 | 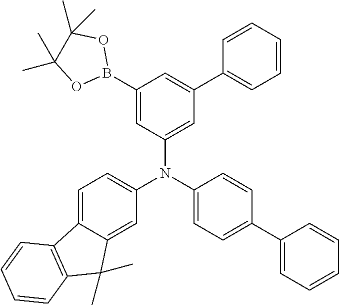 [SMILES: CC1(C)c2ccccc2-c2ccc(N(c3ccc(-c4ccccc4)cc3)c3cc(B4OC(C)(C)C(C)(C)O4)cc(-c4ccccc4)c3)cc21] | 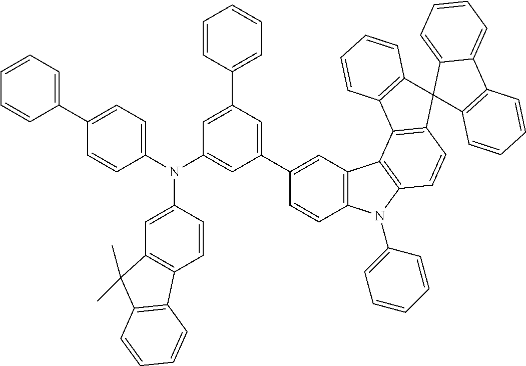 [SMILES: CC1(C)c2ccccc2-c2ccc(N(c3ccc(-c4ccccc4)cc3)c3cc(-c4ccccc4)cc(-c4ccc5c(c4)c4c6c(ccc4n5-c4ccccc4)C4(c5ccccc5-c5ccccc54)c4ccccc4-6)c3)cc21] | 76% |
| Com 37-18 | 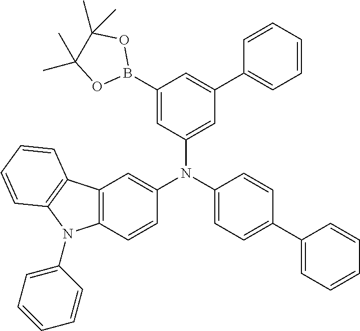 [SMILES: CC1(C)OB(c2cc(-c3ccccc3)cc(N(c3ccc(-c4ccccc4)cc3)c3ccc4c(c3)c3ccccc3n4-c3ccccc3)c2)OC1(C)C] | 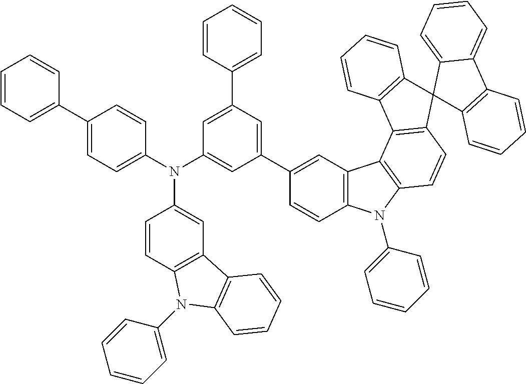 [SMILES: c1ccc(-c2ccc(N(c3cc(-c4ccccc4)cc(-c4ccc5c(c4)c4c6c(ccc4n5-c4ccccc4)C4(c5ccccc5-c5ccccc54)c4ccccc4-6)c3)c3ccc4c(c3)c3ccccc3n4-c3ccccc3)cc2)cc1] | 58% |
| Com 37-19 | 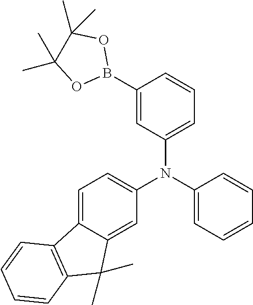 [SMILES: CC1(C)c2ccccc2-c2ccc(N(c3ccccc3)c3cccc(B4OC(C)(C)C(C)(C)O4)c3)cc21] | 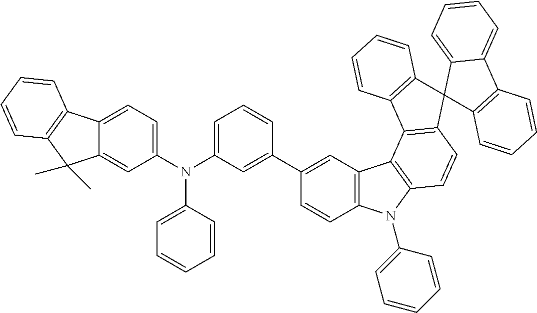 [SMILES: CC1(C)c2ccccc2-c2ccc(N(c3ccccc3)c3cccc(-c4ccc5c(c4)c4c6c(ccc4n5-c4ccccc4)C4(c5ccccc5-c5ccccc54)c4ccccc4-6)c3)cc21] | 65% |
| Com 37-20 | 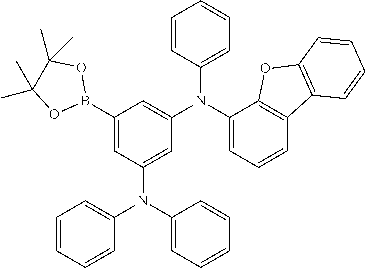 [SMILES: CC1(C)OB(c2cc(N(c3ccccc3)c3ccccc3)cc(N(c3ccccc3)c3cccc4c3oc3ccccc34)c2)OC1(C)C] | 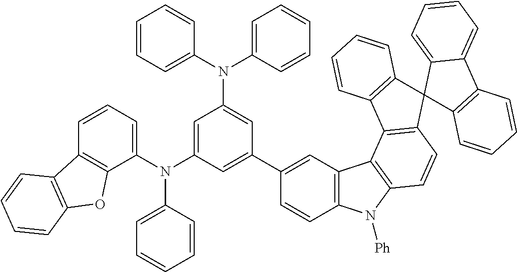 [SMILES: c1ccc(N(c2ccccc2)c2cc(-c3ccc4c(c3)c3c5c(ccc3n4-c3ccccc3)C3(c4ccccc4-c4ccccc43)c3ccccc3-5)cc(N(c3ccccc3)c3cccc4c3oc3ccccc34)c2)cc1] | 61% |
| Com 37-21 | 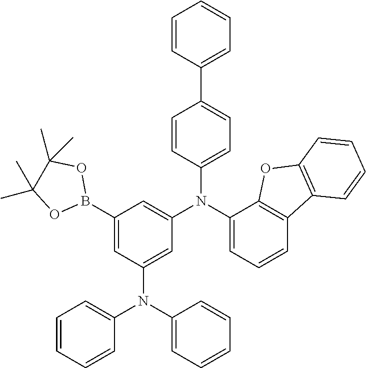 [SMILES: CC1(C)OB(c2cc(N(c3ccccc3)c3ccccc3)cc(N(c3ccc(-c4ccccc4)cc3)c3cccc4c3oc3ccccc34)c2)OC1(C)C] | 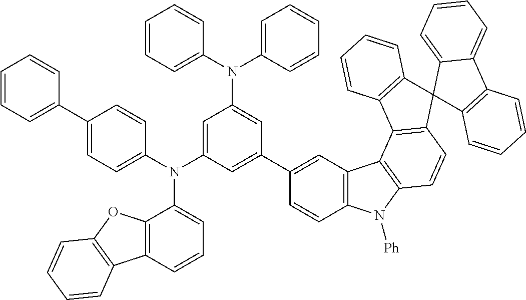 [SMILES: c1ccc(-c2ccc(N(c3cc(-c4ccc5c(c4)c4c6c(ccc4n5-c4ccccc4)C4(c5ccccc5-c5ccccc54)c4ccccc4-6)cc(N(c4ccccc4)c4ccccc4)c3)c3cccc4c3oc3ccccc34)cc2)cc1] | 77% |
| Com 37-22 | 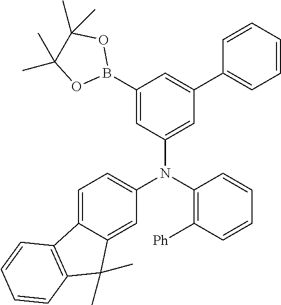 [SMILES: CC1(C)c2ccccc2-c2ccc(N(c3cc(B4OC(C)(C)C(C)(C)O4)cc(-c4ccccc4)c3)c3ccccc3-c3ccccc3)cc21] | 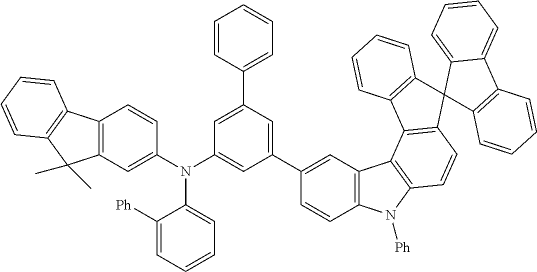 [SMILES: CC1(C)c2ccccc2-c2ccc(N(c3cc(-c4ccccc4)cc(-c4ccc5c(c4)c4c6c(ccc4n5-c4ccccc4)C4(c5ccccc5-c5ccccc54)c4ccccc4-6)c3)c3ccccc3-c3ccccc3)cc21] | 69% |
| Com 37-23 | 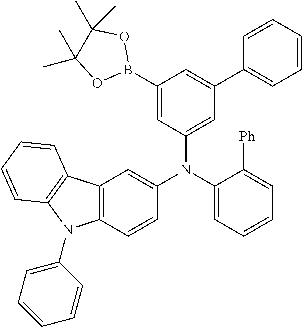 [SMILES: CC1(C)OB(c2cc(-c3ccccc3)cc(N(c3ccc4c(c3)c3ccccc3n4-c3ccccc3)c3ccccc3-c3ccccc3)c2)OC1(C)C] | 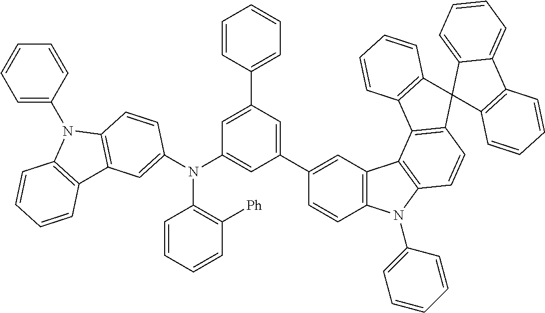 [SMILES: c1ccc(-c2cc(-c3ccc4c(c3)c3c5c(ccc3n4-c3ccccc3)C3(c4ccccc4-c4ccccc43)c3ccccc3-5)cc(N(c3ccc4c(c3)c3ccccc3n4-c3ccccc3)c3ccccc3-c3ccccc3)c2)cc1] | 81% |
| Com 37-24 | 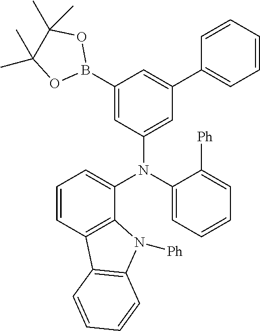 [SMILES: CC1(C)OB(c2cc(-c3ccccc3)cc(N(c3ccccc3-c3ccccc3)c3cccc4c5ccccc5n(-c5ccccc5)c34)c2)OC1(C)C] | 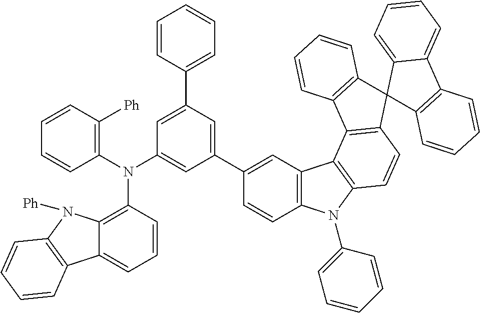 [SMILES: c1ccc(-c2cc(-c3ccc4c(c3)c3c5c(ccc3n4-c3ccccc3)C3(c4ccccc4-c4ccccc43)c3ccccc3-5)cc(N(c3ccccc3-c3ccccc3)c3cccc4c5ccccc5n(-c5ccccc5)c34)c2)cc1] | 75% |
| Com 37-25 | 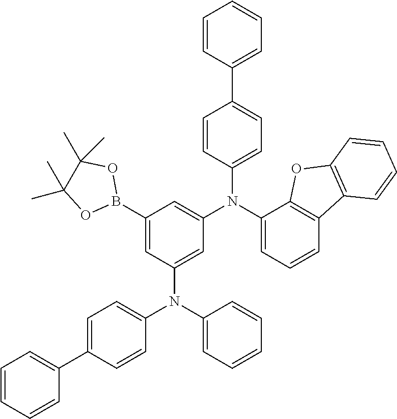 [SMILES: CC1(C)OB(c2cc(N(c3ccccc3)c3ccc(-c4ccccc4)cc3)cc(N(c3ccc(-c4ccccc4)cc3)c3cccc4c3oc3ccccc34)c2)OC1(C)C] | 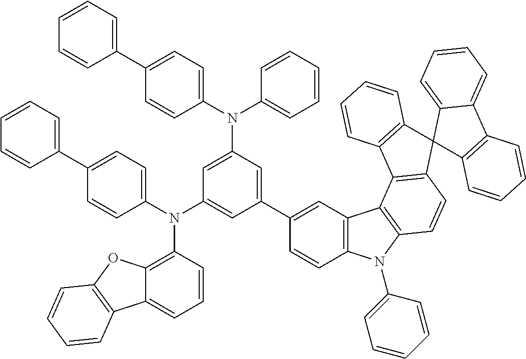 [SMILES: c1ccc(-c2ccc(N(c3ccccc3)c3cc(-c4ccc5c(c4)c4c6c(ccc4n5-c4ccccc4)C4(c5ccccc5-c5ccccc54)c4ccccc4-6)cc(N(c4ccc(-c5ccccc5)cc4)c4cccc5c4oc4ccccc45)c3)cc2)cc1] | 80% |
| Com 37-26 | 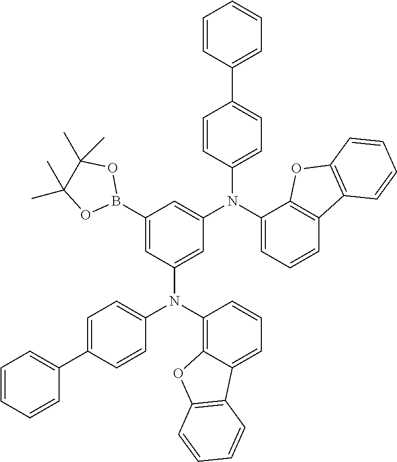 [SMILES: CC1(C)OB(c2cc(N(c3ccc(-c4ccccc4)cc3)c3cccc4c3oc3ccccc34)cc(N(c3ccc(-c4ccccc4)cc3)c3cccc4c3oc3ccccc34)c2)OC1(C)C] | 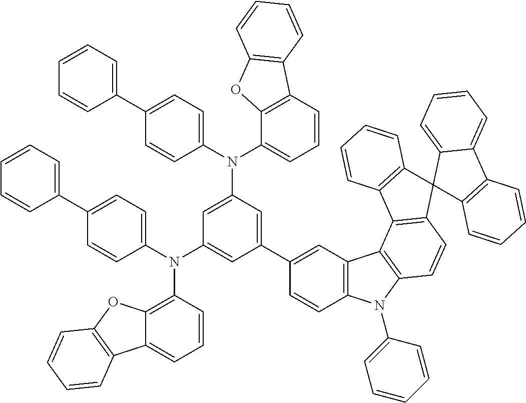 [SMILES: c1ccc(-c2ccc(N(c3cc(-c4ccc5c(c4)c4c6c(ccc4n5-c4ccccc4)C4(c5ccccc5-c5ccccc54)c4ccccc4-6)cc(N(c4ccc(-c5ccccc5)cc4)c4cccc5c4oc4ccccc45)c3)c3cccc4c3oc3ccccc34)cc2)cc1] | 62% |
| Com 37-27 | 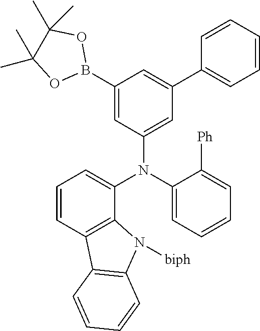 | 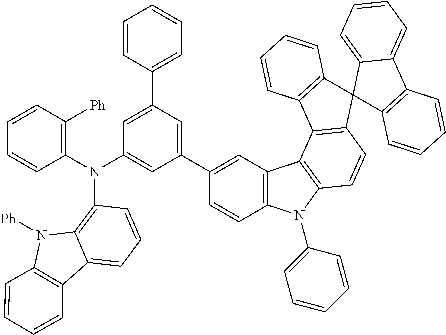 [SMILES: c1ccc(-c2cc(-c3ccc4c(c3)c3c5c(ccc3n4-c3ccccc3)C3(c4ccccc4-c4ccccc43)c3ccccc3-5)cc(N(c3ccccc3-c3ccccc3)c3cccc4c5ccccc5n(-c5ccccc5)c34)c2)cc1] | 61% |
| Com 37-28 | 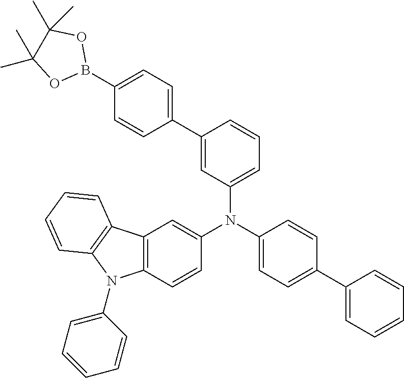 [SMILES: CC1(C)OB(c2ccc(-c3cccc(N(c4ccc(-c5ccccc5)cc4)c4ccc5c(c4)c4ccccc4n5-c4ccccc4)c3)cc2)OC1(C)C] | 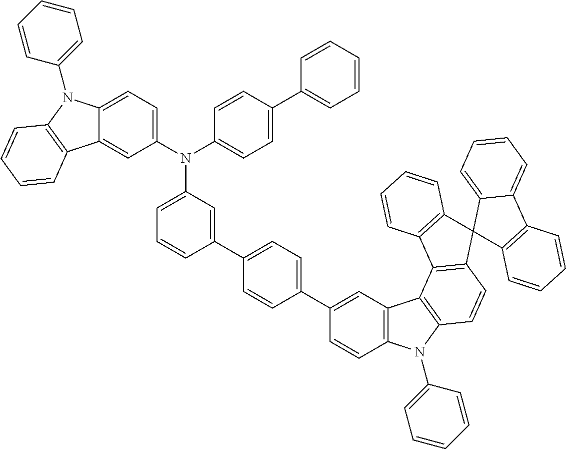 [SMILES: c1ccc(-c2ccc(N(c3cccc(-c4ccc(-c5ccc6c(c5)c5c7c(ccc5n6-c5ccccc5)C5(c6ccccc6-c6ccccc65)c5ccccc5-7)cc4)c3)c3ccc4c(c3)c3ccccc3n4-c3ccccc3)cc2)cc1] | 71% |
| Com 37-29 | 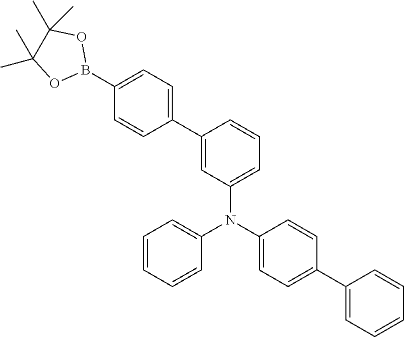 [SMILES: CC1(C)OB(c2ccc(-c3cccc(N(c4ccccc4)c4ccc(-c5ccccc5)cc4)c3)cc2)OC1(C)C] | 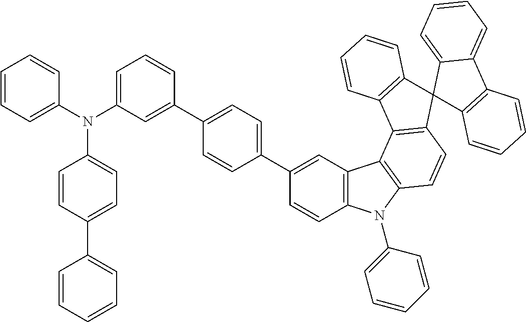 [SMILES: c1ccc(-c2ccc(N(c3ccccc3)c3cccc(-c4ccc(-c5ccc6c(c5)c5c7c(ccc5n6-c5ccccc5)C5(c6ccccc6-c6ccccc65)c5ccccc5-7)cc4)c3)cc2)cc1] | 68% |
| Com 37-30 | 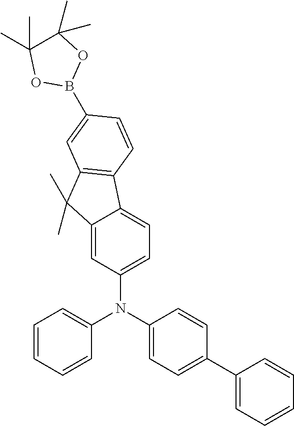 [SMILES: CC1(C)c2cc(B3OC(C)(C)C(C)(C)O3)ccc2-c2ccc(N(c3ccccc3)c3ccc(-c4ccccc4)cc3)cc21] | 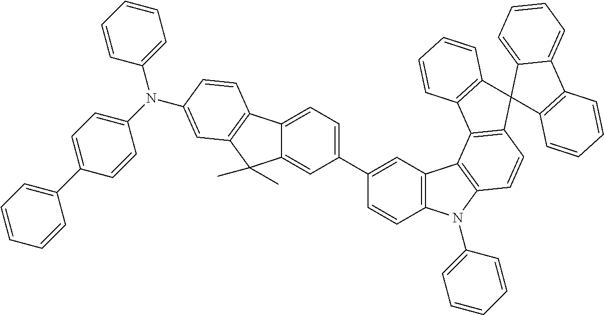 [SMILES: CC1(C)c2cc(-c3ccc4c(c3)c3c5c(ccc3n4-c3ccccc3)C3(c4ccccc4-c4ccccc43)c3ccccc3-5)ccc2-c2ccc(N(c3ccccc3)c3ccc(-c4ccccc4)cc3)cc21] | 69% |
| Com 37-31 | 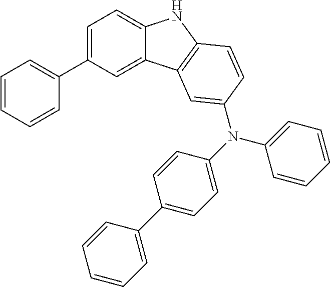 [SMILES: c1ccc(-c2ccc(N(c3ccccc3)c3ccc4[nH]c5ccc(-c6ccccc6)cc5c4c3)cc2)cc1] | 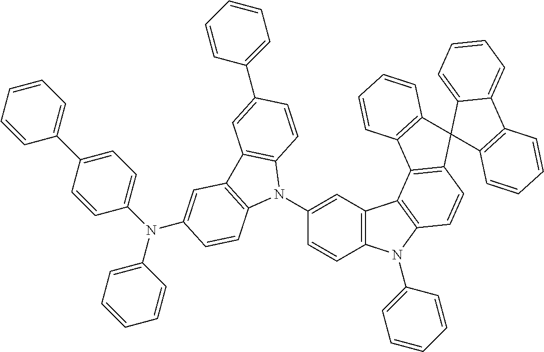 [SMILES: c1ccc(-c2ccc(N(c3ccccc3)c3ccc4c(c3)c3cc(-c5ccccc5)ccc3n4-c3ccc4c(c3)c3c5c(ccc3n4-c3ccccc3)C3(c4ccccc4-c4ccccc43)c3ccccc3-5)cc2)cc1] | 71% |
| Com 37-32 | 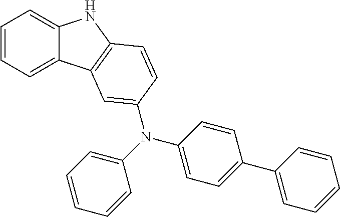 [SMILES: c1ccc(-c2ccc(N(c3ccccc3)c3ccc4[nH]c5ccccc5c4c3)cc2)cc1] | 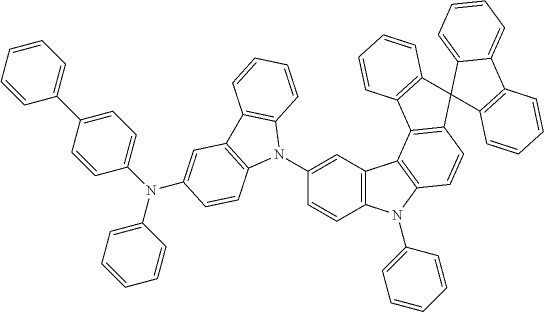 [SMILES: c1ccc(-c2ccc(N(c3ccccc3)c3ccc4c(c3)c3ccccc3n4-c3ccc4c(c3)c3c5c(ccc3n4-c3ccccc3)C3(c4ccccc4-c4ccccc43)c3ccccc3-5)cc2)cc1] | 72% |
| Com 37-33 | 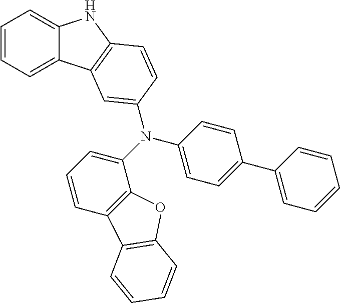 [SMILES: c1ccc(-c2ccc(N(c3ccc4[nH]c5ccccc5c4c3)c3cccc4c3oc3ccccc34)cc2)cc1] | 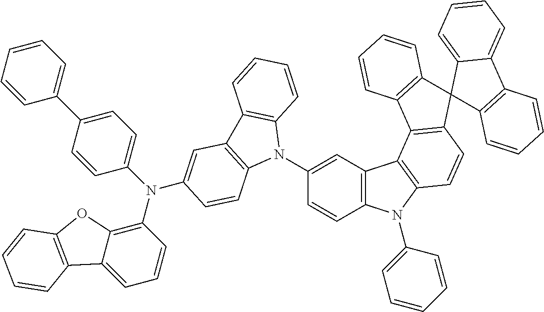 [SMILES: c1ccc(-c2ccc(N(c3ccc4c(c3)c3ccccc3n4-c3ccc4c(c3)c3c5c(ccc3n4-c3ccccc3)C3(c4ccccc4-c4ccccc43)c3ccccc3-5)c3cccc4c3oc3ccccc34)cc2)cc1] | 70% |
| Com 37-34 | 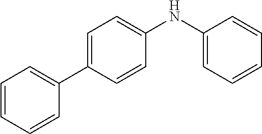 [SMILES: c1ccc(Nc2ccc(-c3ccccc3)cc2)cc1] | 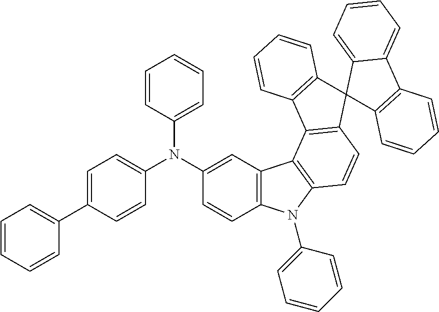 [SMILES: c1ccc(-c2ccc(N(c3ccccc3)c3ccc4c(c3)c3c5c(ccc3n4-c3ccccc3)C3(c4ccccc4-c4ccccc43)c3ccccc3-5)cc2)cc1] | 58% |
| Com 37-35 | 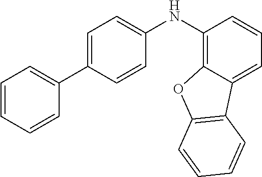 [SMILES: c1ccc(-c2ccc(Nc3cccc4c3oc3ccccc34)cc2)cc1] | 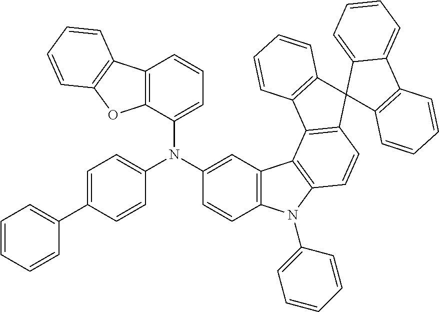 [SMILES: c1ccc(-c2ccc(N(c3ccc4c(c3)c3c5c(ccc3n4-c3ccccc3)C3(c4ccccc4-c4ccccc43)c3ccccc3-5)c3cccc4c3oc3ccccc34)cc2)cc1] | 54% |
| Com 37-36 | 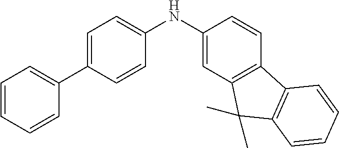 [SMILES: CC1(C)c2ccccc2-c2ccc(Nc3ccc(-c4ccccc4)cc3)cc21] | 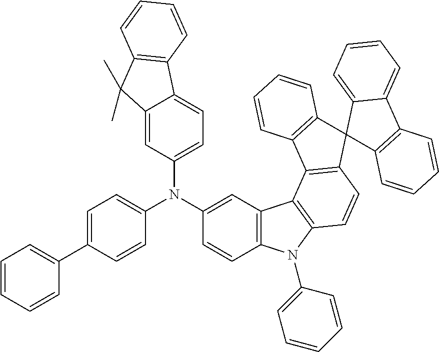 [SMILES: CC1(C)c2ccccc2-c2ccc(N(c3ccc(-c4ccccc4)cc3)c3ccc4c(c3)c3c5c(ccc3n4-c3ccccc3)C3(c4ccccc4-c4ccccc43)c3ccccc3-5)cc21] | 65% |
| Com 37-37 | 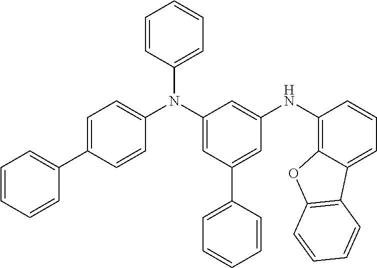 [SMILES: c1ccc(-c2ccc(N(c3ccccc3)c3cc(Nc4cccc5c4oc4ccccc45)cc(-c4ccccc4)c3)cc2)cc1] | 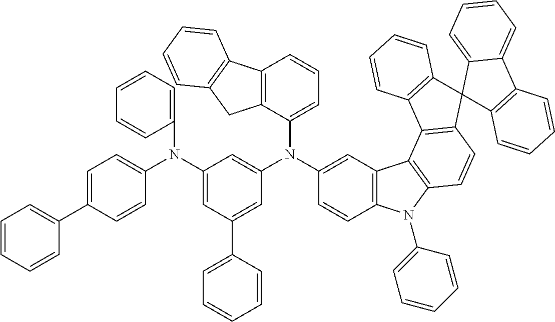 [SMILES: c1ccc(-c2ccc(N(c3ccccc3)c3cc(-c4ccccc4)cc(N(c4ccc5c(c4)c4c6c(ccc4n5-c4ccccc4)C4(c5ccccc5-c5ccccc54)c4ccccc4-6)c4cccc5c4Cc4ccccc4-5)c3)cc2)cc1] | 59% |
| Com 37-38 | 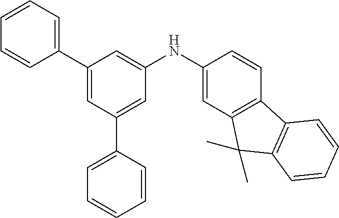 [SMILES: CC1(C)c2ccccc2-c2ccc(Nc3cc(-c4ccccc4)cc(-c4ccccc4)c3)cc21] | 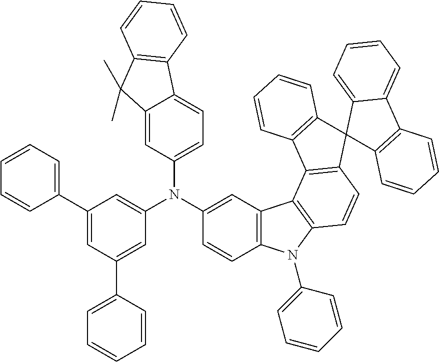 [SMILES: CC1(C)c2ccccc2-c2ccc(N(c3cc(-c4ccccc4)cc(-c4ccccc4)c3)c3ccc4c(c3)c3c5c(ccc3n4-c3ccccc3)C3(c4ccccc4-c4ccccc43)c3ccccc3-5)cc21] | 64% |
| Com 37-39 | 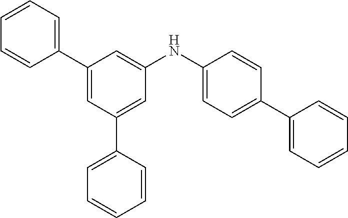 [SMILES: c1ccc(-c2ccc(Nc3cc(-c4ccccc4)cc(-c4ccccc4)c3)cc2)cc1] | 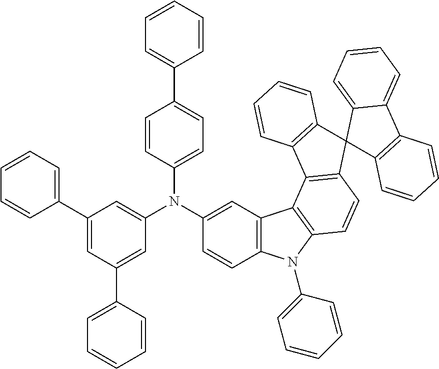 [SMILES: c1ccc(-c2ccc(N(c3cc(-c4ccccc4)cc(-c4ccccc4)c3)c3ccc4c(c3)c3c5c(ccc3n4-c3ccccc3)C3(c4ccccc4-c4ccccc43)c3ccccc3-5)cc2)cc1] | 75% |
| Com 37-40 | 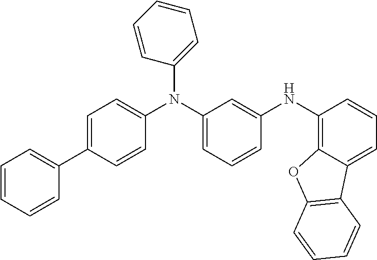 [SMILES: c1ccc(-c2ccc(N(c3ccccc3)c3cccc(Nc4cccc5c4oc4ccccc45)c3)cc2)cc1] | 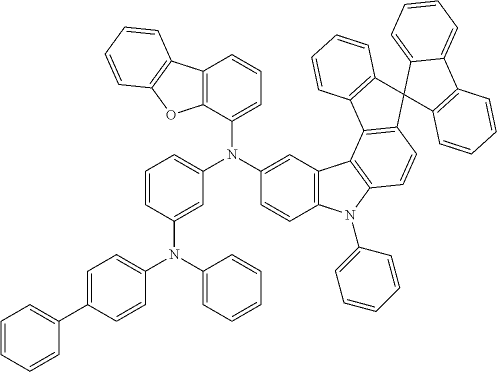 [SMILES: c1ccc(-c2ccc(N(c3ccccc3)c3cccc(N(c4ccc5c(c4)c4c6c(ccc4n5-c4ccccc4)C4(c5ccccc5-c5ccccc54)c4ccccc4-6)c4cccc5c4oc4ccccc45)c3)cc2)cc1] | 74% |
| Com 37-41 | 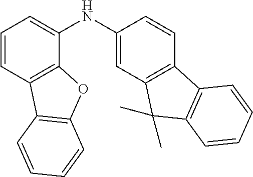 [SMILES: CC1(C)c2ccccc2-c2ccc(Nc3cccc4c3oc3ccccc34)cc21] | 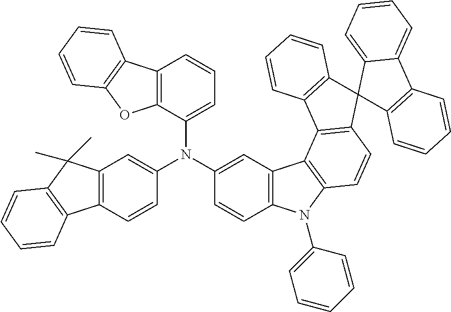 [SMILES: CC1(C)c2ccccc2-c2ccc(N(c3ccc4c(c3)c3c5c(ccc3n4-c3ccccc3)C3(c4ccccc4-c4ccccc43)c3ccccc3-5)c3cccc4c3oc3ccccc34)cc21] | 68% |
| Com 37-42 | 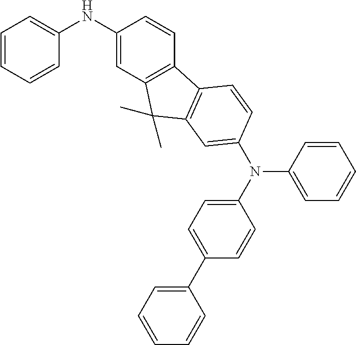 [SMILES: CC1(C)c2cc(Nc3ccccc3)ccc2-c2ccc(N(c3ccccc3)c3ccc(-c4ccccc4)cc3)cc21] | 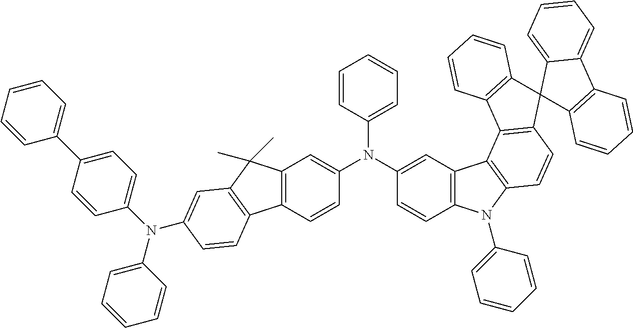 [SMILES: CC1(C)c2cc(N(c3ccccc3)c3ccc(-c4ccccc4)cc3)ccc2-c2ccc(N(c3ccccc3)c3ccc4c(c3)c3c5c(ccc3n4-c3ccccc3)C3(c4ccccc4-c4ccccc43)c3ccccc3-5)cc21] | 71% |
| Com 37-43 | 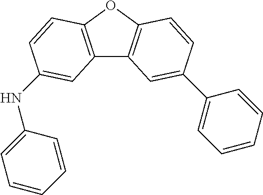 [SMILES: c1ccc(Nc2ccc3oc4ccc(-c5ccccc5)cc4c3c2)cc1] | 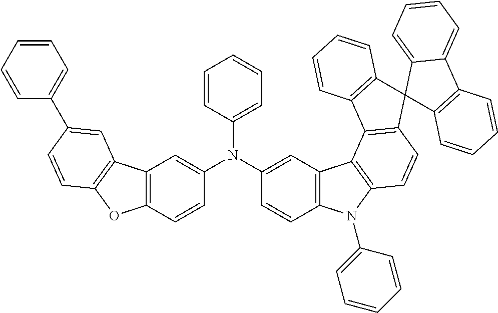 [SMILES: c1ccc(-c2ccc3oc4ccc(N(c5ccccc5)c5ccc6c(c5)c5c7c(ccc5n6-c5ccccc5)C5(c6ccccc6-c6ccccc65)c5ccccc5-7)cc4c3c2)cc1] | 64% |
| Com 37-44 | 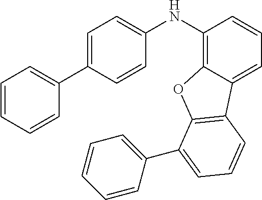 [SMILES: c1ccc(-c2ccc(Nc3cccc4c3oc3c(-c5ccccc5)cccc34)cc2)cc1] | 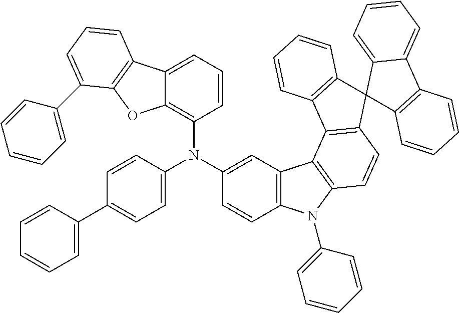 [SMILES: c1ccc(-c2ccc(N(c3ccc4c(c3)c3c5c(ccc3n4-c3ccccc3)C3(c4ccccc4-c4ccccc43)c3ccccc3-5)c3cccc4c3oc3c(-c5ccccc5)cccc34)cc2)cc1] | 64% |
| Com 37-45 | 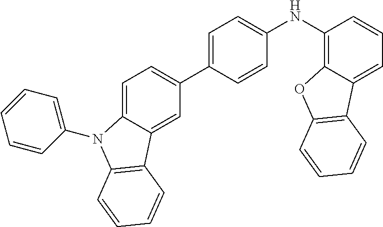 [SMILES: c1ccc(-n2c3ccccc3c3cc(-c4ccc(Nc5cccc6c5oc5ccccc56)cc4)ccc32)cc1] | 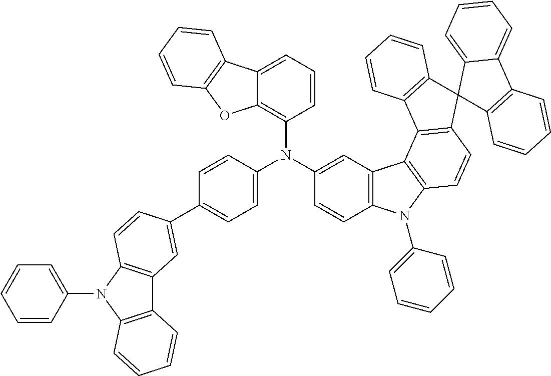 [SMILES: c1ccc(-n2c3ccccc3c3cc(-c4ccc(N(c5ccc6c(c5)c5c7c(ccc5n6-c5ccccc5)C5(c6ccccc6-c6ccccc65)c5ccccc5-7)c5cccc6c5oc5ccccc56)cc4)ccc32)cc1] | 66% |
| Com 37-46 | 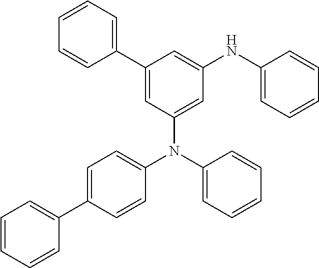 [SMILES: c1ccc(Nc2cc(-c3ccccc3)cc(N(c3ccccc3)c3ccc(-c4ccccc4)cc3)c2)cc1] | 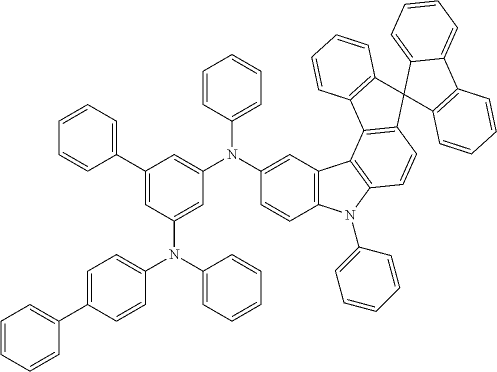 [SMILES: c1ccc(-c2ccc(N(c3ccccc3)c3cc(-c4ccccc4)cc(N(c4ccccc4)c4ccc5c(c4)c4c6c(ccc4n5-c4ccccc4)C4(c5ccccc5-c5ccccc54)c4ccccc4-6)c3)cc2)cc1] | 65% |
| Com 37-47 | 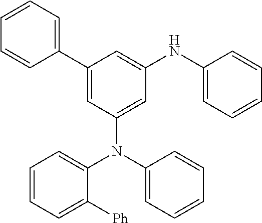 [SMILES: c1ccc(Nc2cc(-c3ccccc3)cc(N(c3ccccc3)c3ccccc3-c3ccccc3)c2)cc1] | 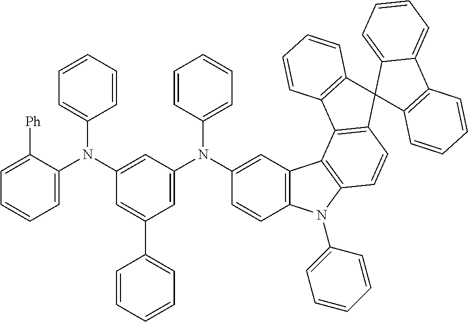 [SMILES: c1ccc(-c2cc(N(c3ccccc3)c3ccc4c(c3)c3c5c(ccc3n4-c3ccccc3)C3(c4ccccc4-c4ccccc43)c3ccccc3-5)cc(N(c3ccccc3)c3ccccc3-c3ccccc3)c2)cc1] | 70% |
| Com 37-48 | 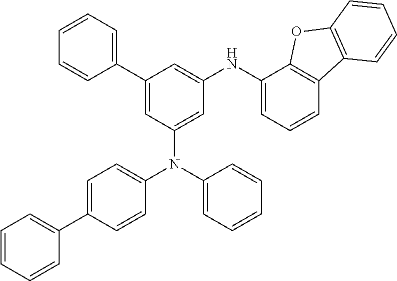 [SMILES: c1ccc(-c2ccc(N(c3ccccc3)c3cc(Nc4cccc5c4oc4ccccc45)cc(-c4ccccc4)c3)cc2)cc1] | 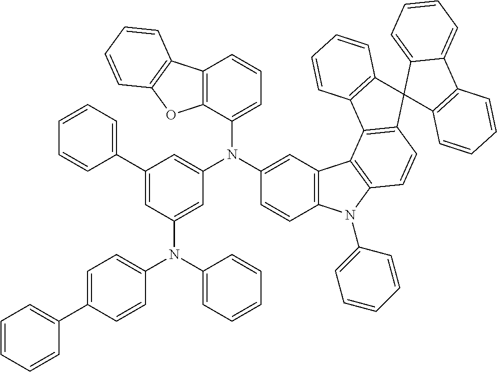 [SMILES: c1ccc(-c2ccc(N(c3ccccc3)c3cc(-c4ccccc4)cc(N(c4ccc5c(c4)c4c6c(ccc4n5-c4ccccc4)C4(c5ccccc5-c5ccccc54)c4ccccc4-6)c4cccc5c4oc4ccccc45)c3)cc2)cc1] | 74% |
| Com 37-49 | 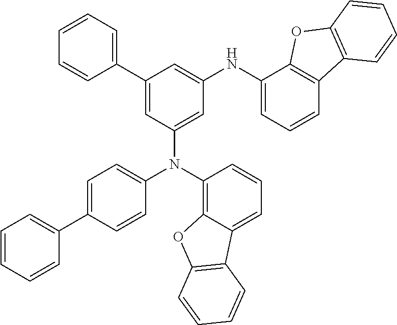 [SMILES: c1ccc(-c2ccc(N(c3cc(Nc4cccc5c4oc4ccccc45)cc(-c4ccccc4)c3)c3cccc4c3oc3ccccc34)cc2)cc1] | 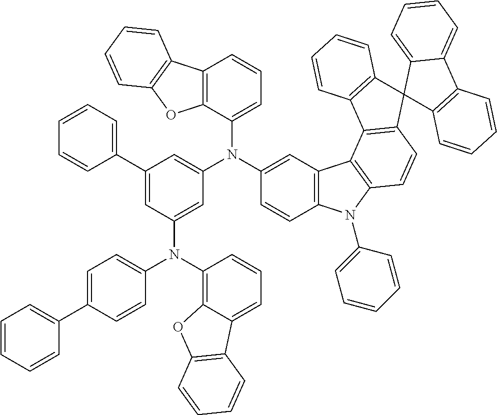 [SMILES: c1ccc(-c2ccc(N(c3cc(-c4ccccc4)cc(N(c4ccc5c(c4)c4c6c(ccc4n5-c4ccccc4)C4(c5ccccc5-c5ccccc54)c4ccccc4-6)c4cccc5c4oc4ccccc45)c3)c3cccc4c3oc3ccccc34)cc2)cc1] | 69% |
| Com 37-50 | 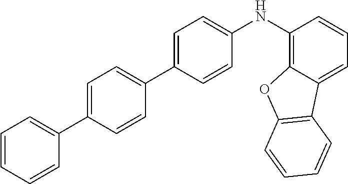 [SMILES: c1ccc(-c2ccc(-c3ccc(Nc4cccc5c4oc4ccccc45)cc3)cc2)cc1] | 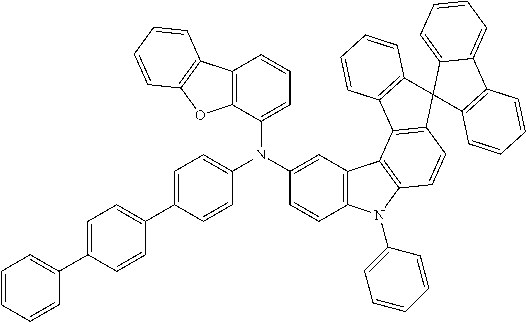 [SMILES: c1ccc(-c2ccc(-c3ccc(N(c4ccc5c(c4)c4c6c(ccc4n5-c4ccccc4)C4(c5ccccc5-c5ccccc54)c4ccccc4-6)c4cccc5c4oc4ccccc45)cc3)cc2)cc1] | 70% |
| Com 37-51 | 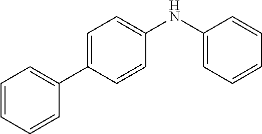 [SMILES: c1ccc(Nc2ccc(-c3ccccc3)cc2)cc1] | 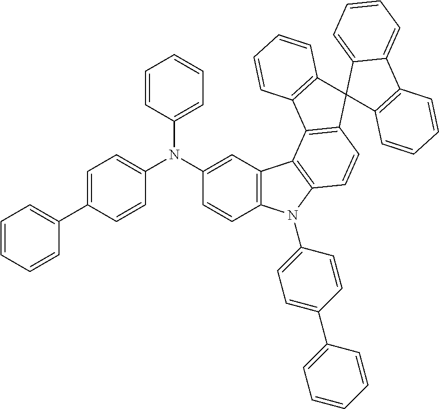 [SMILES: c1ccc(-c2ccc(N(c3ccccc3)c3ccc4c(c3)c3c5c(ccc3n4-c3ccc(-c4ccccc4)cc3)C3(c4ccccc4-c4ccccc43)c3ccccc3-5)cc2)cc1] | 70% |
| Com 37-52 | 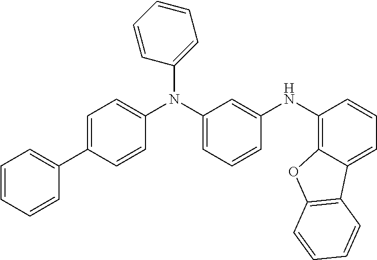 [SMILES: c1ccc(-c2ccc(N(c3ccccc3)c3cccc(Nc4cccc5c4oc4ccccc45)c3)cc2)cc1] | 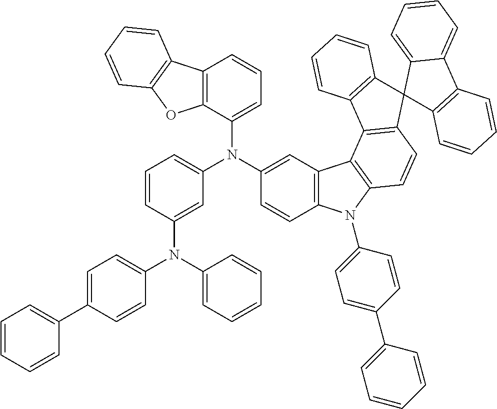 [SMILES: c1ccc(-c2ccc(N(c3ccccc3)c3cccc(N(c4ccc5c(c4)c4c6c(ccc4n5-c4ccc(-c5ccccc5)cc4)C4(c5ccccc5-c5ccccc54)c4ccccc4-6)c4cccc5c4oc4ccccc45)c3)cc2)cc1] | 61% |
| Com 37-53 | 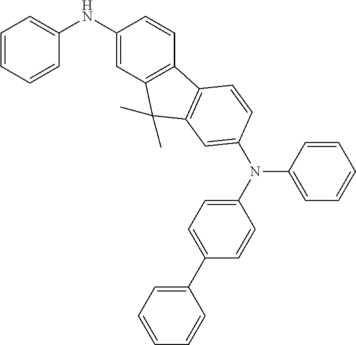 [SMILES: CC1(C)c2cc(Nc3ccccc3)ccc2-c2ccc(N(c3ccccc3)c3ccc(-c4ccccc4)cc3)cc21] | 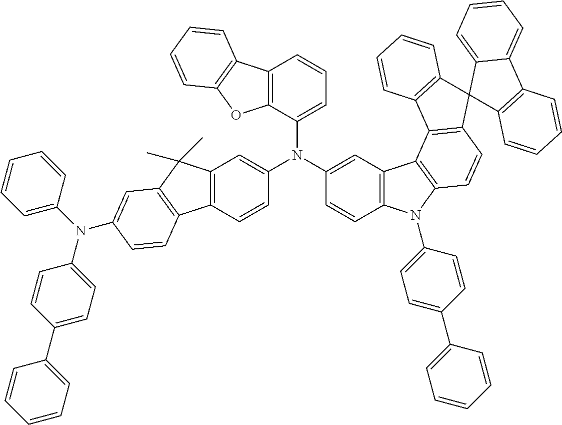 [SMILES: CC1(C)c2cc(N(c3ccccc3)c3ccc(-c4ccccc4)cc3)ccc2-c2ccc(N(c3ccc4c(c3)c3c5c(ccc3n4-c3ccc(-c4ccccc4)cc3)C3(c4ccccc4-c4ccccc43)c3ccccc3-5)c3cccc4c3oc3ccccc34)cc21] | 68% |
| Com 37-54 | 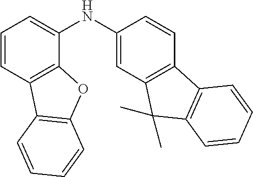 [SMILES: CC1(C)c2ccccc2-c2ccc(Nc3cccc4c3oc3ccccc34)cc21] | 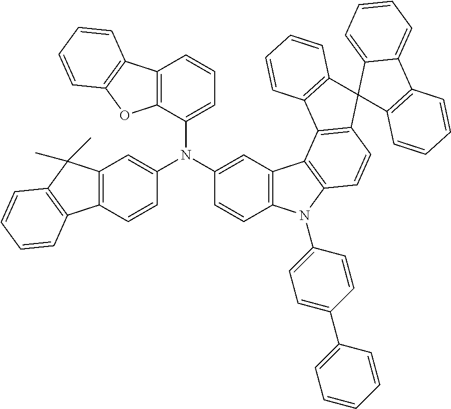 [SMILES: CC1(C)c2ccccc2-c2ccc(N(c3ccc4c(c3)c3c5c(ccc3n4-c3ccc(-c4ccccc4)cc3)C3(c4ccccc4-c4ccccc43)c3ccccc3-5)c3cccc4c3oc3ccccc34)cc21] | 57% |
| Com 37-55 | 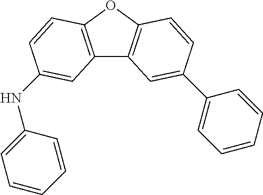 [SMILES: c1ccc(Nc2ccc3oc4ccc(-c5ccccc5)cc4c3c2)cc1] | 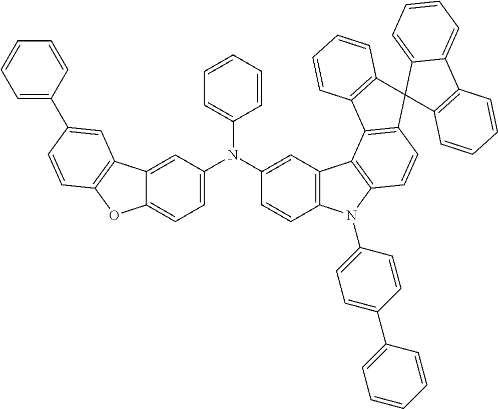 [SMILES: c1ccc(-c2ccc(-n3c4ccc(N(c5ccccc5)c5ccc6oc7ccc(-c8ccccc8)cc7c6c5)cc4c4c5c(ccc43)C3(c4ccccc4-c4ccccc43)c3ccccc3-5)cc2)cc1] | 60% |
| Com 37-56 | 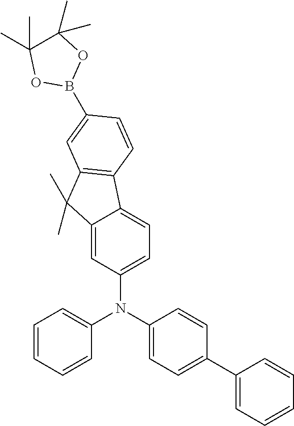 [SMILES: CC1(C)c2cc(B3OC(C)(C)C(C)(C)O3)ccc2-c2ccc(N(c3ccccc3)c3ccc(-c4ccccc4)cc3)cc21] | 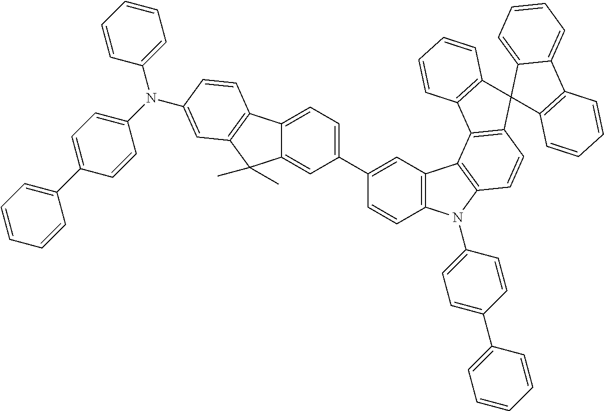 [SMILES: CC1(C)c2cc(-c3ccc4c(c3)c3c5c(ccc3n4-c3ccc(-c4ccccc4)cc3)C3(c4ccccc4-c4ccccc43)c3ccccc3-5)ccc2-c2ccc(N(c3ccccc3)c3ccc(-c4ccccc4)cc3)cc21] | 65% |
| Com 37-57 | 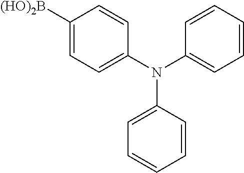 [SMILES: OB(O)c1ccc(N(c2ccccc2)c2ccccc2)cc1] | 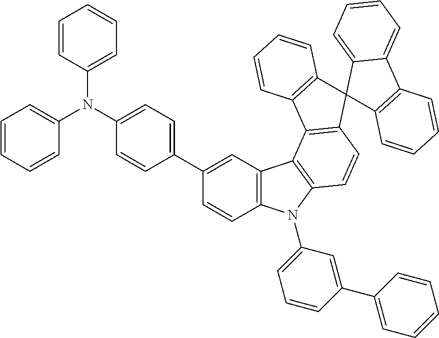 [SMILES: c1ccc(-c2cccc(-n3c4ccc(-c5ccc(N(c6ccccc6)c6ccccc6)cc5)cc4c4c5c(ccc43)C3(c4ccccc4-c4ccccc43)c3ccccc3-5)c2)cc1] | 62% |
| Com 37-58 | 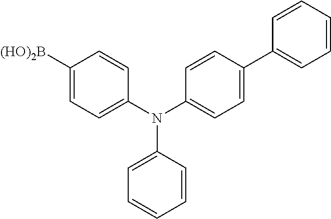 [SMILES: OB(O)c1ccc(N(c2ccccc2)c2ccc(-c3ccccc3)cc2)cc1] | 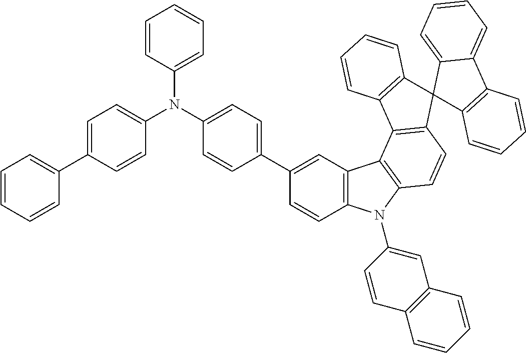 [SMILES: c1ccc(-c2ccc(N(c3ccccc3)c3ccc(-c4ccc5c(c4)c4c6c(ccc4n5-c4ccc5ccccc5c4)C4(c5ccccc5-c5ccccc54)c4ccccc4-6)cc3)cc2)cc1] | 65% |
| Com 37-59 | 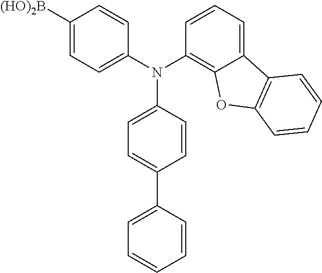 [SMILES: OB(O)c1ccc(N(c2ccc(-c3ccccc3)cc2)c2cccc3c2oc2ccccc23)cc1] | 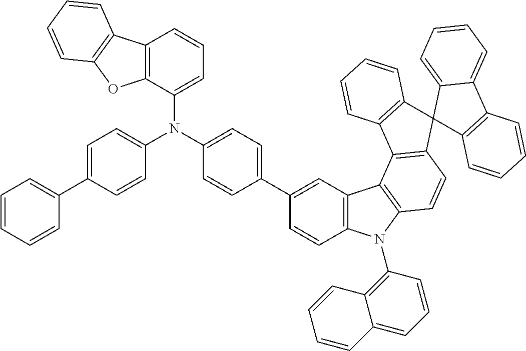 [SMILES: c1ccc(-c2ccc(N(c3ccc(-c4ccc5c(c4)c4c6c(ccc4n5-c4cccc5ccccc45)C4(c5ccccc5-c5ccccc54)c4ccccc4-6)cc3)c3cccc4c3oc3ccccc34)cc2)cc1] | 68% |

The ¹H NMR and MS values of the compounds prepared in Examples described above are shown in Table 8.

TABLE 8

| Compound | | MS | |
|---|---|---|---|
| No. | NMR | found | calculated |
| Com. | δ = 8.24(1H, d), 8.08(1H, d), 7.89(2H, m), 7.79(1H, s), 7.74(1H, | 675.88 | 675.29 |
| 21-1 | d), 7.66(2H, m), 7.59(4H, m), 7.45(1H, d), 7.37(5H, m), | | |
| | 7.26(7H, m), 7.08(4H, d), 7.00(2H, t), 1.69(6H, s) | | |
| Com. | δ = 8.24(1H, d), 8.08(1H, d), 7.89(2H, m), 7.79(1H, s), 7.78(1H, | 725.94 | 725.31 |
| 21-2 | d), 7.74(1H, d), 7.71(1H, d), 7.66(2H, m), 7.55(5H, m), 7.45- | | |
| | 7.24(14H, m), 7.11-7.00(4H, m), 1.69(6H, s) | | |
| Com. | δ = 8.24(1H, d), 8.08(1H, d), 7.89(2H, m), 7.79-7.27(29H, m), | 776.00 | 775.32 |
| 21-3 | 7.11(2H, s), 1.69(6H, s) | | |
| Com. | δ = 8.24(1H, d), 8.08(1H, d), 7.89(2H, m), 7.79(1H, s). 7.75(5H, | 751.97 | 751.32 |
| 21-4 | m), 7.68(1H, d), 7.64(1H, d), 7.56(7H, m), 7.49-7.41(4H, m), | | |
| | 7.37(7H, m), 7.26(5H, m), 7.08(2H, d), 7.00(1H, t), 1.69(6H, s) | | |
| Com. | δ = 8.24(1H, d), 8.08(1H, d), 7.89(2H, m), 7.79(1H, s), 7.75(5H, | 828.07 | 827.36 |
| 21-5 | m), 7.68(1H, d), 7.64(1H, d), 7.56(8H, m), 7.49-7.41(6H, m), | | |
| | 7.37(8H, m), 7.26(3H, m), 1.69(6H, s) | | |
| Com. | δ = 8.24(1H, d), 8.08(1H, d), 7.88(4H, m), 7.79-7.27(30H, m), | 868.14 | 867.39 |
| 21-6 | 7.16(1H, d), 1.69(12H, s) | | |
| Com. | δ = 8.24(1H, dd), 8.08(1H, d), 7.98(1H, dd), 7.89(2H, m), | 842.05 | 841.33 |
| 21-7 | 7.79(1H, s), 7.74(3H, m), 7.68(1H, d), 7.64(2H, m), 7.56(7H, m), | | |
| | 7.47(3H, m), 7.41-7.37(9H, m), 7.31-7.27(5H, m), 6.97(1H, dd), | | |
| | 1.69(6H, s) | | |
| Com. | δ = 8.24(1H, dd), 8.10(1H, d), 8.08(1H, d), 7.98(1H, d), | 842.05 | 841.33 |
| 21-8 | 7.89(2H, m), 7.79(1H, s), 7.74(1H, d), 7.68-7.54(8H, m), 7.45- | | |
| | 7.27(17H, m), 7.14-7.08(3H, m), 6.97(1H, dd), 1.69(6H, s) | | |
| Com. | δ = 8.24(1H, dd), 8.08(3H, m), 7.89(6H, m), 7.78-7.37(32H, m), | 1136.50 | 1135.51 |
| 21-9 | 7.27(5H, m), 1.69(186H, s) | | |
| Com. | δ = 8.24(1H, dd), 8.08(1H, d), 7.89(2H, m), 7.79(1H, sd), | 904.17 | 903.39 |
| 21-10 | 7.74(7H, m), 7.68-7.54(9H, m), 7.49-7.27(22H, m), 1.69(6H, s) | | |
| Com. | δ = 8.24(1H, d), 8.08(1H, d), 7.89(2H, m), 7.79(1H, s), 7.74(3H, | 751.97 | 751.32 |
| 21-11 | m), 7.64(3H, m), 7.56(2H, m), 7.45(3H, m), 7.38-7.24(13H, m), | | |
| | 7.08-7.00(6H, m) 1.69(6H, s) | | |
| Com. | δ = 8.25(1H, dd), 8.08(1H, d), 7.89(2H, m), 7.79(1H, s), | 828.07 | 827.36 |
| 21-12 | 7.74(5H, m), 7.68(1H, d), 7.64(1H, d), 7.60(1H, s), 7.56(4H, m), | | |
| | 7.49-7.41(7H, m), 7.37(7H, m), 7.27(3H, m), 7.24(2H, m), | | |
| | 7.08(2H, d), 7.00(1H, t), 1.69(6H, s) | | |
| Com. | δ = 8.25(1H, dd), 8.08(1H, d), 7.98(1H, dd), 7.89(2H, m), | 918.15 | 917.37 |
| 21-13 | 7.79(1H, sd), 7.74(5H, m), 7.68(1H, d), 7.64(2H, d), 7.60(1H, s), | | |
| | 7.56(5H, m), 7.49-7.41(7H, m), 7.37(8H, m), 7.29(5H, m), | | |
| | 6.97(1H, d), 1.69(6H, s) | | |
| Com. | δ = 8.25(1H, d), 8.08(1H, d), 7.98(1H, dd), 7.89(2H, m), | 918.15 | 917.37 |
| 21-14 | 7.79(1H, sd), 7.74(5H, m), 7.68(1H, d), 7.64(1H, d), 7.60(1H, s), | | |
| | 7.56(5H, m), 7.49-7.41(7H, m), 7.35(10H, m), 7.27(4H, m), | | |
| | 6.91(1H, d), 1.70(6H, s) | | |
| Com. | δ = 8.95(1H, d), 8.50(1H, d), 8.22(2H, m), 8.08(2H, m), | 968.21 | 967.38 |
| 21-15 | 7.98(1H, d), 7.89(2H, m), 7.79(1H, s), 7.74(4H, m), 7.68(1H, d), | | |
| | 7.64(1H, d), 7.60(1H, s), 7.55(6H, m), 7.49-7.41(4H, m), | | |
| | 7.38(9H, m), 7.34-7.25(6H, m), 6.91(1H, d), 1.70(6H, s) | | |
| Com. | δ = 8.95(1H, d), 8.50(1H, d), 8.22(2H, m), 8.08(2H, m), | 968.21 | 967.38 |
| 21-16 | 7.98(1H, d), 7.89(2H, m), 7.79(1H, s), 7.76(4H, m), 7.64(4H, m), | | |
| | 7.53(6H, m), 7.49-7.41(4H, m), 7.37(9H, m), 7.31-7.27(5H, m), | | |
| | 6.97(1H, d), 1.70(6H, s) | | |
| Com. | δ = 8.24(1H, d), 8.08(1H, d), 7.88(4H, m), 7.79(1H, s), 7.75(5H, | 944.23 | 943.42 |
| 21-17 | m), 7.64(3H, m), 7.56(5H, m), 7.49-7.41(7H, m), 7.37(8H, m), | | |
| | 7.33(1H, s), 7.27(4H, m), 7.16(1H, d), 1.70(12H, s) | | |
| Com. | δ = 8.24(1H, d), 8.19(1H, d), 8.08(1H, d), 8.01(1H, s), 7.89(3H, | 917.17 | 916.38 |
| 21-18 | m), 7.79(1H, s), 7.74(3H, m), 7.64(5H, m), 7.56(4H, m), 7.37(5H, | | |
| | m), 7.28-7.20(6H, m), 7.08(2H, d), 7.00(1H, t), 6.48(1H, d), | | |
| | 1.70(6H, s) | | |
| Com. | δ = 8.24(1H, d), 8.08(1H, d), 7.88(4H, m), 7.79(1H, s), 7.74(1H, | 792.04 | 791.36 |
| 21-19 | d), 7.68(1H, d), 7.64(1H, d), 7.56(4H, m), 7.45(1H, m), 7.38(4H, | | |
| | m), 7.33(1H, s), 7.28-7.24(7H, m), 7.17(3H, m), 7.08(2H, d), | | |
| | 7.00(1H, t), 1.68(12H, s) | | |
| Com. | δ = 8.24(1H, d), 8.08(1H, d), 7.98(1H, dd), 7.88(2H, m), | 933.17 | 932.38 |
| 21-20 | 7.79(1H, s), 7.74(1H, d), 7.68(1H, d), 7.64(2H, d), 7.56(3H, m), | | |
| | 7.45(1H, m), 7.38(4H, m), 7.31-7.24(11H, m), 7.08(6H, d), 7.00- | | |
| | 6.97(4H, m), 6.93(3H, s), 1.68(6H, s) | | |
| Com. | δ = 8.24(1H, d), 8.08(1H, d), 7.98(1H, d), 7.89(2H, m), 7.79(1H, | 1009.27 | 1008.41 |
| 21-21 | s), 7.74(3H, m), 7.68(1H, d), 7.64(2H, d), 7.56(5H, m), 7.49- | | |
| | 7.41(4H, m), 7.39-7.24(15H, m), 7.08(4H, d), 7.00-6.97(3H, m), | | |
| | 6.93(3H, s), 1.68(6H, s) | | |
| Com. | δ = 8.24(1H, d), 8.09(2H, m), 7.90-7.86(4H, m), 7.79(1H, s), | 944.23 | 943.42 |
| 21-22 | 7.74(3H, m), 7.68(1H, d), 7.64(1H, d), 7.60(1H, s), 7.56(3H, m), | | |
| | 7.49-7.41(7H, m), 7.39-7.37(8H, m), 7.33(1H, d), 7.27(4H, m), | | |
| | 7.15(2H, m), 7.08(2H, m), 1.68(12H, s) | | |
| Com. | δ = 8.24(1H, d), 8.19(1H, d), 8.09(2H, m), 8.01(1H, s), 7.89(3H, | 993.27 | 992.41 |
| 21-23 | m), 7.79(1H, s), 7.74(3H, m), 7.68(1H, d), 7.64-7.62(3H, d), | | |
| | 7.60(1H, s), 7.57(4H, m), 7.50-7.41(10H, m), 7.39-7.37(7H, m), | | |
| | 7.27(3H, m), 7.20-7.14(2H, m), 7.08(2H, m), 6.48(1H, d), | | |
| | 1.68(6H, s) | | |
| Com. | δ = 8.24(1H, d), 8.21(1H, d), 8.19(1H, d), 8.09(2H, m), 7.89(2H, | 993.27 | 992.41 |
| 21-24 | m), 7.79(1H, s), 7.74(3H, m), 7.68(1H, d), 7.64-7.62(3H, m), | | |
| | 7.60(1H, s), 7.57(4H, m), 7.50-7.41(10H, m), 7.39-7.37(7H, m), | | |
| | 7.27(3H, m), 7.20-7.14(2H, m), 7.08(2H, m), 6.48(1H, d), | | |
| | 6.22(1H, d), 1.68(6H, s) | | |
| Com. | δ = 8.24(1H, d), 8.08(1H, d), 7.98(1H, d), 7.89(2H, m), 7.79(1H, | 1085.36 | 1084.44 |
| 21-25 | s), 7.74(6H, m), 7.68(1H, d), 7.64(2H, d), 7.55(7H, m), 7.50- | | |
| | 7.41(7H, m), 7.39-7.37(8H, m), 7.31-7.27(5H, m), 7.24(2H, m), | | |
| | 7.08(2H, d), 7.00-6.97(2H, d), 6.93(3H, s), 1.68(6H, s) | | |
| Com. | δ = 8.24(1H, d), 8.08(1H, d), 7.98(2H, d), 7.89(2H, m), 7.79(1H, | 1175.44 | 1174.45 |
| 21-26 | s), 7.74(5H, m), 7.68(1H, d), 7.64(2H, d), 7.55(7H, m), 7.50- | | |
| | 7.41(7H, m), 7.39-7.37(8H, m), 7.31-7.27(5H, m), 7.24(2H, m), | | |
| | 7.08(2H, d), 7.00-6.97(2H, d), 6.93(3H, s), 1.68(6H, s) | | |
| Com. | δ = 8.24-8.19(3H, m), 8.09(2H, m), 7.89(2H, m), 7.79(1H, s), | 993.27 | 992.41 |
| 21-27 | 7.74(3H, m), 7.68(1H, d), 7.64(1H, d), 7.62(2H, m), 7.60(1H, s), | | |
| | 7.58-7.55(4H, m), 7.50-7.41(10H, m), 7.39-7.37(7H, m), 7.31- | | |
| | 7.27(3H, m), 7.20(1H, m), 7.14(1H, m), 7.08(2H, m), 6.48(1H, d), | | |
| | 6.22(1H, t), 1.68(6H, s) | | |
| Com. | δ = 8.24(1H, d), 8.19(1H, d), 8.08(1H, d), 8.01(1H, s), 7.89(3H, | 993.27 | 992.41 |
| 21-28 | m), 7.79(1H, s), 7.74(3H, m), 7.68(1H, d), 7.64(1H, d), 7.62(2H, | | |
| | m), 7.58-7.55(7H, m), 7.50-7.41(7H, m), 7.39-7.37(5H, m), 7.28- | | |
| | 7.25(8H, m), 7.20-7.18(3H, m), 6.48(1H, d), 1.68(6H, s) | | |
| Com. | δ = 8.24(1H, d), 8.08(1H, d), 7.89(2H, m), 7.79(1H, s), 7.78- | 828.07 | 827.36 |
| 21-29 | 7.75(3H, m), 7.68(1H, d), 7.64(1H, d), 7.58-7.55(5H, m), 7.50- | | |
| | 7.41(4H, m), 7.39-7.37(5H, m), 7.28-7.17(12H, m), 7.08(2H, d), | | |
| | 7.00(1H, t), 1.98(6H, s) | | |
| Com. | δ = 8.24(1H, d), 8.08(2H, d), 7.90-7.86(4H, m), 7.79(1H, s), | 868.14 | 867.59 |
| 21-30 | 7.78-7.74(4H, m), 7.68(1H, d), 7.64(1H, d), 7.57-7.55(4H, m), | | |
| | 7.50-7.41(6H, m), 7.39-7.37(3H, m), 7.33(1H, s), 7.28-7.16(6H, | | |
| | m), 7.08(2H, d), 7.00(1H, t), 1.98(12H, s) | | |
| Com. | δ = 8.30(1H, d), 8.24(1H, d), 8.13(1H, d), 8.03(1H, d), 7.89(3H, | 917.17 | 916.38 |
| 21-31 | m), 7.74(5H, m), 7.64(1H, d), 7.57-7.55(6H, m), 7.49-7.41(8H, | | |
| | m), 7.38-7.24(12H, m), 7.08(2H, d), 7.00(1H, t), 1.98(6H, s) | | |
| Com. | δ = 8.55(1H, d), 8.24(1H, d), 8.03(1H, d), 7.94-7.89(3H, m), | 841.07 | 840.35 |
| 21-32 | 7.74(3H, m), 7.64(2H, d), 7.57-7.54(6H, m), 7.49-7.40(5H, m), | | |
| | 7.39-7.24(14H, m), 7.16(1H, t), 7.08(2H, d), 7.00(1H, t), 1.98(6H, | | |
| | s) | | |
| Com. | δ = 8.55(1H, d), 8.24(1H, d), 8.03(1H, d), 7.98-7.89(3H, m), | 931.15 | 930.36 |
| 21-33 | 7.74(3H, m), 7.64(2H, d), 7.57-7.54(7H, m), 7.49-7.40(5H, m), | | |
| | 7.38-7.24(15H, m), 7.16(1H, t), 6.97(1H, d), 1.98(6H, s) | | |
| Com. | δ = 8.24(1H, d), 7.9-7.85(3H, m), 7.74(3H, m), 7.64(1H, d), | 675.88 | 675.29 |
| 21-34 | 7.57-7.54(4H, m), 7.49-7.40(4H, m), 7.38-7.23(11H, m), 7.08- | | |
| | 7.00(4H, m), 1.69(6H, s) | | |
| Com. | δ = 8.24(1H, d), 7.98(1H, d), 7.90-7.85(3H, m), 7.74(3H, m), | 765.96 | 765.30 |
| 21-35 | 7.64(2H, d), 7.57-7.54(5H, m), 7.49-7.40(4H, m), 7.38-7.23(12H, | | |
| | m), 7.06(1H, d), 6.97(1H, d), 1.69(6H, s) | | |
| Com. | δ = 8.24(1H, d), 7.90-7.85(5H, m), 7.74(3H, m), 7.64(1H, d), | 792.04 | 791.36 |
| 21-36 | 7.57(1H, t), 7.55(4H, d), 7.49-7.41(4H, m), 7.38(3H, t), 7.37(3H, | | |
| | d), 7.33(1H, s), 7.27(4H, m), 7.23(1H, s), 7.16(1H, d), 7.06(1H, | | |
| | d), 1.69(12H, s) | | |
| Com. | δ = 8.24(1H, d), 7.98(1H, d), 7.90-7.85(3H, m), 7.74(5H, m), | 1009.27 | 1008.41 |
| 21-37 | 7.64(2H, d), 7.60-7.55(6H, m), 7.49-7.231(7H, m), 7.39-7.37(6H, | | |
| | t), 7.31-7.23(8H, m), 7.08-6.97(4H, m), 7.00(1H, t), 6.93(3H, s), | | |
| | 1.69(6H, s) | | |
| Com. | δ = 8.24(1H, d), 7.90-7.85(5H, m), 7.74(5H, m), 7.64(1H, d), | 868.14 | 867.39 |
| 21-38 | 7.57-7.54(5H, m), 7.49-7.23(18H, m), 7.16(1H, d), 7.06(1H, d), | | |
| | 1.69(12H, s) | | |
| Com. | 8 8.24(1H, d), 7.90-7.85(3H, m), 7.74(7H, m), 7.64(1H, d), | 828.07 | 827.36 |
| 21-39 | 7.60(1H, s), 7.57-7.54(4H, m), 7.49-7.23(21H, m), 7.06(1H, d), | | |
| | 1.69(6H, s) | | |
| Com. | δ = 8.24(1H, d), 7.98(1H, d), 7.90-7.85(3H, m), 7.74(3H, m), | 933.17 | 932.38 |
| 21-40 | 7.64(2H, d), 7.57-7.54(5H, m), 7.49-7.23(19H, m), 7.08-6.97(5H, | | |
| | m), 6.83(1H, s), 6.74(2H, d), 1.69(6H, s) | | |
| Com. | δ = 8.24(1H, d), 7.98(1H, d), 7.90-7.85(5H, m), 7.74(1H, d), | 806.02 | 805.33 |
| 21-41 | 7.64(2H, d), 7.57-7.54(4H, m), 7.45-7.23(14H, m), 7.16(1H, d), | | |
| | 7.06(1H, d), 6.97(1H, d), 1.69(12H, s) | | |
| Com. | δ = 8.24(1H, d), 7.90-7.85(5H, m), 7.74(3H, m), 7.64(1H, d), | 959.25 | 958.43 |
| 21-42 | 7.57-7.55(4H, m), 7.49-7.41(4H, m), 7.38-7.33(6H, m), 7.28- | | |
| | 7.23(9H, m), 7.16(2H, d), 7.08-7.00(7H, m), 1.69(12H, s) | | |
| Com. | δ = 8.24-8.22(2H, m), 7.90-7.74(9H, m), 7.64(1H, d), 7.57- | 765.96 | 765.30 |
| 21-43 | 7.55(3H, m), 7.49-7.41(4H, m), 7.38-7.37(3H, m), 7.28-7.23(6H, | | |
| | m), 7.08-6.97(5H, m), 1.69(6H, s) | | |
| Com. | δ = 8.24(1H, d), 8.08(1H, d), 8.02(1H, d), 7.90-7.85(3H, m), | 842.05 | 841.33 |
| 21-44 | 7.74(3H, m), 7.64(2H, d), 7.57-7.23(24H, m), 7.06(1H, d), | | |
| | 6.97(1H, d), 1.69(6H, s) | | |
| Com. | δ = 8.30(1H, d), 8.24(1H, d), 8.19(1H, d), 8.13(1H, d), 7.98(1H, | 931.15 | 930.36 |
| 21-45 | d), 7.90-7.85(4H, m), 7.74(1H, d), 7.64(2H, d), 7.57-7.23(26H, | | |
| | m), 7.06(1H, d), 6.97(1H, d), 1.69(6H, s) | | |
| Com. | δ = 8.24(1H, d), 7.90-7.85(3H, m), 7.74(5H, m), 7.64(1H, d), | 919.18 | 918.40 |
| 21-46 | 7.57-7.23(24H, m), 7.08-7.00(7H, m), 6.93(3H, s), 1.69(6H, s) | | |
| Com. | δ = 8.24(1H, d), 8.10(1H, d), 7.90-7.85(3H, m), 7.74(3H, m), | 919.18 | 918.40 |
| 21-47 | 7.64(1H, d), 7.57-7.23(22H, m), 7.14-7.00(10H, m), 6.93(3H, s), | | |
| | 1.69(6H, s) | | |
| Com. | δ = 8.24(1H, d), 7.98(1H, d), 7.90-7.85(3H, m), 7.74(5H, m), | 1009.27 | 1008.41 |
| 21-48 | 7.64(2H, d), 7.57-7.54(5H, m), 7.49-7.41(7H, m), 7.39-7.00(18H, | | |
| | m), 6.97(1H, m), 6.93(3H, s), 1.69(6H, s) | | |
| Com. | δ = 8.24(1H, d), 7.98(2H, d), 7.90-7.85(3H, m), 7.74(5H, m), | 1099.35 | 1098.42 |
| 21-49 | 7.64(3H, d), 7.57-7.54(6H, m), 7.49-7.41(7H, m), 7.39-7.23(16H, | | |
| | m), 7.06(1H, d), 6.97(2H, d), 6.93(3H, s), 1.69(6H, s) | | |
| Com. | δ = 8.24(1H, d), 7.98(1H, d), 7.90-7.85(3H, m), 7.74(3H, m), | 842.05 | 841.33 |
| 21-50 | 7.64(2H, d), 7.57-7.54(5H, m), 7.49-7.41(4H, m), 7.39-7.23(16H, | | |
| | m), 7.06(1H, d), 6.97(1H, d), 1.69(6H, s) | | |
| Com. | δ = 8.24(1H, d), 7.89(3H, m), 7.74(1H, d), 7.71(1H, s), 7.57(1H, | 675.88 | 675.29 |
| 22-1 | t), 7.55(3H, d), 7.45(2H, m), 7.39-7.23(13H, m), 7.08(4H, d), | | |
| | 7.00(2H, t), 1.69(6H, s) | | |
| Com. | δ = 8.24(1H, d), 7.89(3H, m), 7.78-7.71(4H, m), 7.57-7.54(5H, | 725.94 | 725.31 |
| 22-2 | m), 7.45-7.40(5H, m), 7.38-7.23(11H, m), 7.11(1H, m), 7.08(2H, | | |
| | m), 7.00(1H, t), 1.69(6H, s) | | |
| Com. | δ = 8.24(1H, d), 7.89(3H, m), 7.78-7.71(6H, m), 7.57-7.54(6H, | 776.00 | 775.32 |
| 22-3 | m), 7.45-7.40(8H, m), 7.38-7.37(4H, m), 7.28-7.23(5H, m), 7.11 | | |
| | (2H, s), 1.69(6H, s) | | |
| Com. | δ = 8.24(1H, d), 7.89(3H, m), 7.75-7.71(4H, m), 7.57-7.54(6H, | 751.97 | 751.32 |
| 22-4 | m), 7.49-7.40(5H, m), 7.38-7.37(6H, m), 7.28-7.23(7H, m), | | |
| | 7.08(2H, d), 7.00(1H, t), 1.69(6H, s) | | |
| Com. | δ = 8.24(1H, d), 7.89(3H, m), 7.75-7.71(6H, m), 7.57(1H, t), | 828.07 | 827.36 |
| 22-5 | 7.55(6H, d), 7.49-7.40(8H, m), 7.38-7.37(8H, m), 7.28-7.27(5H, | | |
| | m), 1.69(6H, s) | | |
| Com. | δ = 8.24(1H, d), 7.90-7.86(5H, m), 7.75-7.71(4H, m), 7.57(1H, | 868.14 | 867.39 |
| 22-6 | t), 7.55(6H, d), 7.49-7.41(5H, m), 7.39-7.31(8H, m), 7.28- | | |
| | 7.27(6H, m), 7.16(1H, d), 1.69(12H, s) | | |
| Com. | δ = 8.24(1H, d), 7.98(1H, d), 7.89(3H, m), 7.75-7.74(3H, m), | 842.05 | 841.33 |
| 22-7 | 7.71(1H, s), 7.64(1H, d), 7.57(1H, t), 7.55(6H, d), 7.49-7.41(5H, | | |
| | m), 7.38-7.33(8H, m), 7.28-7.27(6H, m), 6.97(1H, d), 1.69(6H, s) | | |
| Com. | δ = 8.24(1H, d), 8.10(1H, d), 7.98(1H, d), 7.89(3H, m), 7.74(1H, | 842.05 | 841.33 |
| 22-8 | d), 7.71(1H, s), 7.64(1H, d), 7.57-7.55(5H, m), 7.45-7.27(19H, | | |
| | m), 7.14(1H, m), 7.08(2H, d), 6.97(1H, d), 1.69(6H, s) | | |
| Com. | δ = 8.24(1H, d), 8.09(2H, d), 7.89(7H, m), 7.78-7.71(6H, m), | 1136.50 | 1135.51 |
| 22-9 | 7.71(1H, s), 7.60(1H, s), 7.57-7.55(8H, m), 7.49-7.27(21H, m), | | |
| | 1.69(18H, s) | | |
| Com.2 | δ = 8.24(1H, d), 7.89(3H, m), 7.75-7.71(8H, m), 7.57-7.55(6H, | 904.17 | 903.39 |
| 2-10 | m), 7.49-7.27(25H, m), 1.69(6H, s) | | |
| Com. | δ = 8.24(1H, d), 7.89(3H, m), 7.75-7.71(4H, m), 7.60(1H, s), | 751.97 | 751.32 |
| 22-11 | 7.57-7.55(2H, m), 7.49-7.24(18H, m), 7.08(4H, d), 7.00(2H, t), | | |
| | 1.69(6H, s) | | |
| Com. | δ = 8.24(1H, d), 7.89(3H, m), 7.75-7.71(6H, m), 7.60(1H, s), | 828.07 | 827.36 |
| 22-12 | 7.57-7.55(4H, m), 7.49-7.41(8H, m), 7.38-7.24(13H, m), | | |
| | 7.08(2H, d), 7.00(1H, t), 1.69(6H, s) | | |
| Com. | δ = 8.24(1H, d), 7.98(1H, d), 7.89(3H, m), 7.75-7.71(6H, m), | 918.15 | 917.37 |
| 22-13 | 7.64(1H, d), 7.60(1H, s), 7.57-7.54(5H, m), 7.49-7.41(5H, m), | | |
| | 7.39-7.24(17H, m), 6.97(1H, d), 1.69(6H, s) | | |
| Com. | δ = 8.24(1H, d), 7.98(1H, d), 7.89(3H, m), 7.75-7.71(6H, m), | 918.15 | 917.37 |
| 22-14 | 7.60(1H, s), 7.57-7.54(5H, m), 7.49-7.41(9H, m), 7.39-7.24(14H, | | |
| | m), 6.91(1H, d), 1.69(6H, s) | | |
| Com. | δ = 8.95(1H, d), 8.50(1H, d), 8.24-8.20(2H, m), 8.09(1H, d), | 968.21 | 967.38 |
| 22-15 | 7.98(1H, d), 7.89(3H, m), 7.75-7.71(5H, m), 7.60(1H, s), 7.57- | | |
| | 7.54(6H, m), 7.49-7.41(5H, m), 7.39-7.24(16H, m), 6.91(1H, d), | | |
| | 1.69(6H, s) | | |
| Com. | δ = 8.95(1H, d), 8.50(1H, d), 8.24-8.20(2H, m), 8.09(1H, d), | 968.21 | 967.38 |
| 22-16 | 7.98(1H, d), 7.89(3H, m), 7.77-7.71(5H, m), 7.64(1H, d), 7.60 | | |
| | (1H, s), 7.57-7.52(6H, m), 7.49-7.41(5H, m), 7.39-7.27(15H, m), | | |
| | 6.97(1H, d), 1.69(6H, s) | | |
| Com. | δ = 8.24(1H, d), 7.90-7.86(5H, m), 7.75-7.71(6H, m), 7.60(1H, | 944.23 | 943.42 |
| 22-17 | s), 7.57-7.55(7H, m), 7.49-7.41(7H, m), 7.39-7.27(13H, m), | | |
| | 7.16(1H, d), 1.69(12H, s) | | |
| Com. | δ = 8.55(1H, d), 8.24(1H, d), 7.94-7.89(4H, m), 7.75-7.71(6H, | 993.27 | 992.41 |
| 22-18 | m), 7.62-7.60(3H, m), 7.58-7.54(7H, m), 7.50-7.41(9H, m), | | |
| | 7.39-7.27(14H, m), 7.16(1H, t), 1.69(6H, s) | | |
| Com. | δ = 8.24(1H, d), 7.90-7.86(5H, m), 7.74(1H, d), 7.71(1H, s), | 792.04 | 791.36 |
| 22-19 | 7.57-7.55(4H, m), 7.45-7.16(18H, m), 7.08(2H, d), 7.00(1H, t), | | |
| | 1.69(12H, s) | | |
| Com. | δ = 8.24(1H, d), 7.98(1H, d), 7.90-7.86(3H, m), 7.74(1H, d), | 933.17 | 932.38 |
| 22-20 | 7.71(1H, s), 7.64(1H, d), 7.57-7.54(3H, m), 7.45-7.16(18H, m), | | |
| | 7.08-6.97(10H, m), 6.93(3H, s), 1.69(6H, s) | | |

### [Example 8] Manufacture and Evaluation of Organic Light-Emitting Device

An organic light-emitting device was manufactured according to a common method using each of the compounds obtained in Examples as a hole transport layer.

First, an ITO substrate was installed in a substrate folder of a vacuum deposition equipment, 4,4′,4″-tris[2-naphthyl (phenyl) amino]triphenylamine (2-TNATA) was vacuum-deposited on an ITO layer (anode) formed on a glass substrate to form a hole injection layer having a thickness of 10 nm, and then the spirofluorene compound prepared in each of Examples was vacuum-deposited to form a hole transport layer having a thickness of 20 nm.

Subsequently, BD-331 (Idemitsu Kosan Co., Ltd.) was used as a light-emitting dopant, 9,10-bis(2-naphthyl)anthracene (ADN) was used as a host material, a doping concentration was fixed to 4%, and an emissive layer was deposited on the hole transport layer to a thickness of 30 nm.

Subsequently, tris-(8-hydroxyquinoline)aluminum (Alq3) was vacuum-deposited to a thickness of 40 nm as an electron transport layer on the emissive layer. Thereafter, LiF as an alkali metal halide was deposited to a thickness of 0.2 nm, subsequently, Al was deposited to a thickness of 150 nm, and Al/LiF was used as a cathode, thereby manufacturing an organic light-emitting device.

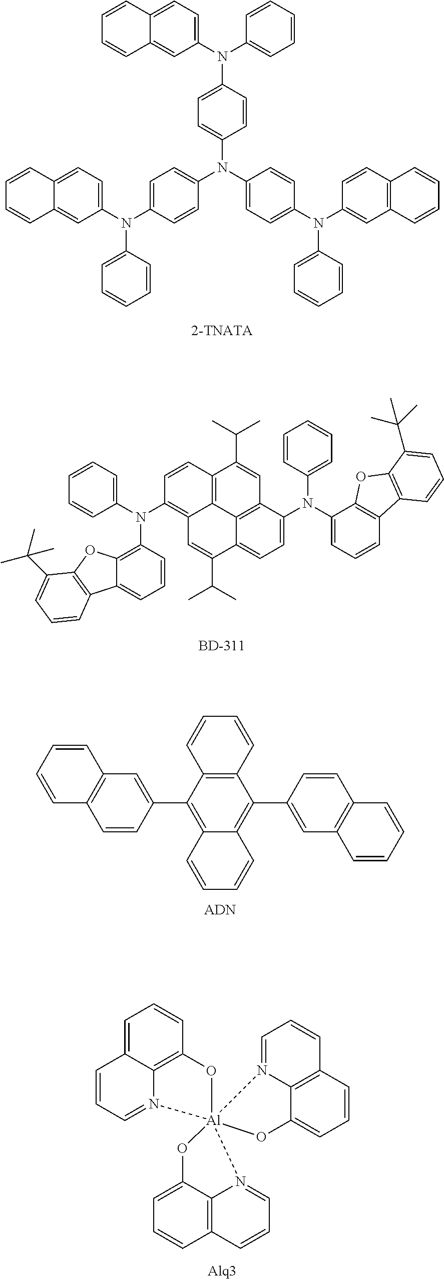

### Comparative Examples 1 to 3

An organic light-emitting device was manufactured in the same manner as that of Example 8, except that the following comparative compound A, comparative compound B, or comparative compound C was used instead of the spirofluorene compound of the present invention as a hole transport material.

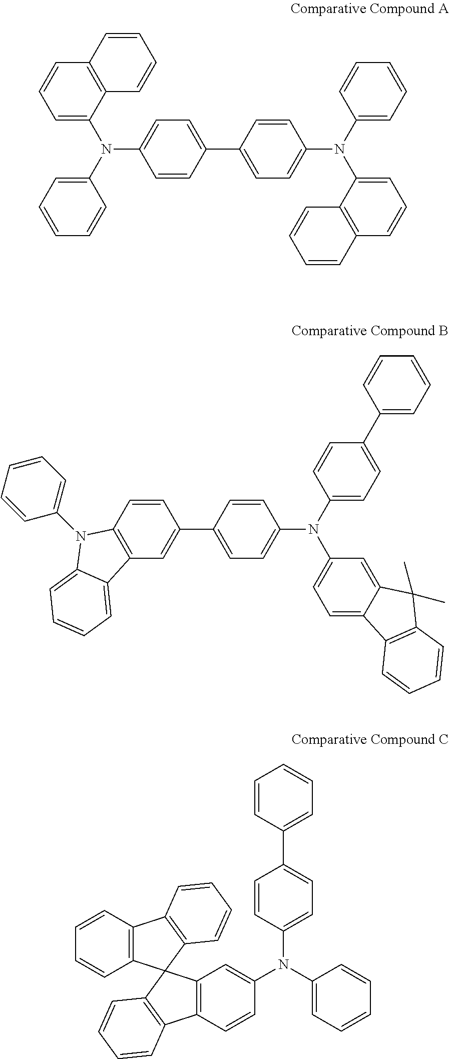 [SMILES: c1ccc(N(c2ccc(-c3ccc(N(c4ccccc4)c4cccc5ccccc45)cc3)cc2)c2cccc3ccccc23)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccccc3)c3ccc4c(c3)C3(c5ccccc5-c5ccccc53)c3ccccc3-4)cc2)cc1]

A forward bias DC voltage was applied to each of the organic light-emitting devices manufactured by Example 8 and Comparative Examples 1 to 3 of the present invention, electroluminescence (EL) characteristics were measured with PR-650 available from Photo Research Inc., and T95 life was measured at a standard luminance of 300 cd/m² by a life measuring equipment manufactured by Mcscience Inc. The measurement results are shown in Table 9. T95 means the time required for the luminance of the light-emitting device to reach 95% of the initial luminance.

TABLE 9

| | Hole transport | Drive | Current | Efficiency | Life |
|---|---|---|---|---|---|
| | material | voltage (V) | (mA/cm²) | (Cd/A) | characteristics |
| | Compound No. | @300 cd/m² | @300 cd/m² | @300 cd/m² | T95 (hr) |
| Example | Com. 21-1 | 4.8 | 6.7 | 5.2 | 90 |
| | Com. 21-2 | 4.6 | 6.9 | 5.6 | 90 |
| | Com. 21-3 | 4.7 | 6.9 | 5.4 | 98 |
| | Com. 21-4 | 4.8 | 5.8 | 5.7 | 115 |
| | Com. 21-5 | 4.8 | 5.9 | 5.7 | 110 |
| | Com. 21-6 | 4.7 | 5.1 | 6.4 | 129 |
| | Com. 21-7 | 4.9 | 5.3 | 6.7 | 123 |
| | Com. 21-8 | 4.8 | 6.0 | 6.2 | 118 |
| | Com. 21-9 | 4.6 | 5.5 | 6.4 | 129 |
| | Com. 21-10 | 4.8 | 5.6 | 6.2 | 108 |
| | Com. 21-11 | 4.8 | 6.1 | 5.1 | 115 |
| | Com. 21-12 | 4.9 | 6.0 | 5.4 | 101 |
| | Com. 21-13 | 4.7 | 6.1 | 5.5 | 107 |
| | Com. 21-14 | 4.7 | 5.1 | 6.3 | 127 |
| | Com. 21-15 | 4.9 | 6.3 | 5.3 | 115 |
| | Com. 21-16 | 4.7 | 5.5 | 6.4 | 127 |
| | Com. 21-17 | 4.7 | 6.3 | 5.2 | 112 |
| | Com. 21-18 | 4.9 | 5.1 | 6.5 | 117 |
| | Com. 21-19 | 4.9 | 5.9 | 6.1 | 101 |
| | Com. 21-20 | 5.1 | 6.8 | 5.0 | 98 |
| | Com. 21-21 | 4.9 | 6.1 | 5.7 | 109 |
| | Com. 21-22 | 4.6 | 6.1 | 5.8 | 102 |
| | Com. 21-23 | 4.7 | 5.8 | 6.2 | 131 |
| | Com. 21-24 | 5.0 | 6.2 | 6.8 | 123 |
| | Com. 21-25 | 4.8 | 6.8 | 5.1 | 115 |
| | Com. 21-26 | 4.9 | 6.8 | 5.3 | 109 |
| | Com. 21-27 | 4.8 | 5.7 | 5.5 | 106 |
| | Com. 21-28 | 4.9 | 5.5 | 5.3 | 112 |
| | Com. 21-29 | 4.6 | 5.8 | 5.6 | 113 |
| | Com. 21-30 | 4.6 | 6.6 | 5.3 | 107 |
| | Com. 21-31 | 4.7 | 6.1 | 5.5 | 110 |
| | Com. 21-32 | 4.7 | 6.0 | 5.9 | 107 |
| | Com. 21-33 | 4.9 | 6.1 | 6.2 | 106 |
| | Com. 21-34 | 4.9 | 6.0 | 5.5 | 104 |
| | Com. 21-35 | 4.8 | 6.6 | 6.3 | 111 |
| | Com. 21-36 | 4.9 | 6.0 | 6.5 | 128 |
| | Com. 21-37 | 4.8 | 6.7 | 5.1 | 100 |
| | Com. 21-38 | 4.9 | 6.8 | 6.3 | 110 |
| | Com. 21-39 | 4.9 | 6.1 | 5.9 | 107 |
| | Com. 21-40 | 5.0 | 6.3 | 6.2 | 106 |
| | Com. 21-41 | 4.7 | 6.1 | 6.3 | 131 |
| | Com. 21-42 | 4.9 | 5.8 | 5.4 | 109 |
| | Com. 21-43 | 5.0 | 5.9 | 5.6 | 113 |
| | Com. 21-44 | 4.9 | 6.4 | 5.1 | 105 |
| | Com. 21-45 | 4.8 | 6.3 | 6.0 | 108 |
| | Com. 21-46 | 4.9 | 7.2 | 4.5 | 98 |
| | Com. 21-47 | 5.0 | 6.3 | 6.1 | 103 |
| | Com. 21-48 | 4.8 | 6.5 | 6.3 | 120 |
| | Com. 21-49 | 4.8 | 5.6 | 5.1 | 100 |
| | Com. 21-50 | 5.0 | 5.7 | 5.2 | 107 |
| | Com. 22-1 | 5.0 | 6.9 | 5.4 | 90 |
| | Com. 22-2 | 5.0 | 6.5 | 5.9 | 100 |
| | Com. 22-3 | 4.8 | 8.2 | 4.2 | 99 |
| | Com. 22-4 | 4.7 | 6.0 | 5.7 | 106 |
| | Com. 22-5 | 4.7 | 5.8 | 5.7 | 101 |
| | Com. 22-6 | 4.8 | 5.7 | 5.5 | 105 |
| | Com. 22-7 | 4.8 | 6.1 | 5.9 | 106 |
| | Com. 22-8 | 4.9 | 6.0 | 5.1 | 107 |
| | Com. 22-9 | 4.9 | 6.5 | 5.8 | 107 |
| | Com. 22-10 | 4.6 | 5.0 | 5.1 | 111 |
| | Com. 22-11 | 4.7 | 6.0 | 5.9 | 107 |
| | Com. 22-12 | 4.9 | 6.1 | 6.2 | 106 |
| | Com. 22-13 | 4.6 | 6.1 | 5.8 | 102 |
| | Com. 22-14 | 5.0 | 6.5 | 5.9 | 100 |
| | Com. 22-15 | 4.9 | 6.0 | 5.1 | 107 |
| | Com. 22-16 | 4.9 | 5.8 | 5.4 | 109 |
| | Com. 22-17 | 4.9 | 5.3 | 6.7 | 123 |
| | Com. 22-18 | 4.9 | 5.1 | 6.5 | 117 |
| | Com. 22-19 | 4.9 | 6.0 | 6.5 | 128 |
| | Com. 22-20 | 4.7 | 6.1 | 5.5 | 107 |
| Comparative | Comparative | 5.4 | 9.6 | 3.1 | 62 |
| Example 1 | Compound A | | | | |
| Comparative | Comparative | 5.5 | 9.5 | 3.2 | 72 |
| Example 2 | Compound B | | | | |
| Comparative | Comparative | 5.1 | 6.9 | 5.1 | 85 |
| Example 3 | Compound C | | | | |

From Table 9, it could be appreciated that the spirofluorene compounds developed in the present invention used as the hole transport materials had light-emitting characteristics superior to those of the hole transport materials according to the related art and also could improve power consumption by inducing an increase in power efficiency through a reduction in drive voltage, and it could be also appreciated that the spirofluorene compounds developed in the present invention were shown to have a remarkable increase in life characteristics.

Therefore, the spirofluorene compound of the present invention is used as a material for forming an organic material layer, such as a hole transport material, such that it is possible to manufacture an organic electroluminescent device exhibiting a low drive voltage, excellent color purity, high emission efficiency, and long-life characteristics. In addition, it is obvious that the same effect may be obtained even when the compounds of the present invention are used in other organic material layers of the organic electroluminescent device, for example, an emissive layer, a hole injection layer, an electron injection layer, an electron transport layer, and the like.

1. A spirofluorene compound represented by the following Chemical Formula 1: 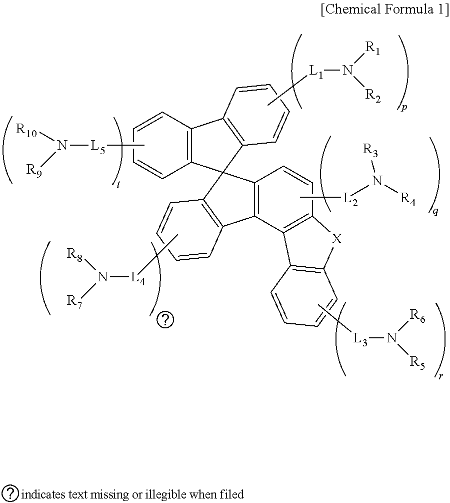
in Chemical Formula 1,
X is CR^{a}R^{b}, NR^{c}, O, S, or SiR^{d}R^{e};
R^{a}, R^{b}, R^{c}, R^{d}, and R^{e} are each independently hydrogen, deuterium, C1-C60 alkyl, C3-C60 cycloalkyl, C6-C60 aryl, or C3-C60 heteroaryl;
L₁ to L₅ are each independently a single bond, C6-C60 arylene, or C3-C60 heteroarylene;
R₁ to R₁₀ are each independently C1-C60 alkyl, C2-C60 alkenyl, C2-C60 alkynyl, C3-C60 cycloalkyl, C2-C60 heterocycloalkyl, C6-C60 aryl, C3-C60 heteroaryl, or -L₁₁-R₁₁;
L₁₁ is C6-C60 arylene or C3-C60 heteroarylene;
R₁₁ is C6-C60 aryl, C3-C60 heteroaryl, or —NR₁₂R₁₃;
R₁₂ and R₁₃ are each independently hydrogen, C1-C60 alkyl, C6-C60 aryl, or C3-C60 heteroaryl;
the arylene and heteroarylene of L₁ to L₅, the alkyl, alkenyl, alkynyl, cycloalkyl, heterocycloalkyl, aryl, and heteroaryl of R₁ to R₁₀, the arylene and heteroarylene of L₁₁, the aryl and heteroaryl of R₁₁, and the alkyl, aryl, and heteroaryl of R₁₂ and R₁₃ may be further substituted by one or more selected from the group consisting of C1-C60 alkyl, halo C1-C60 alkyl, deuterium, halogen, cyano, C3-C60 cycloalkyl, C1-C60 alkoxy, C6-C60 aryl, C6-C60 aryloxy, C6-C60 aryl C1-C60 alkyl, C1-C60 alkyl C6-C60 aryl, C3-C60 heteroaryl, —NR′R″, nitro, and hydroxy;
R′ and R″ are each independently hydrogen, C1-C60 alkyl, C6-C60 aryl, or C3-C60 heteroaryl;
p, r, s, and t are each independently an integer of 0 to 4, and q is an integer of 0 to 2, but p, q, r, s, and t are not simultaneously 0; and
the heteroarylene and heteroaryl contain one or more heteroatoms selected from N, O, S, and Se.

2. The spirofluorene compound of claim 1, wherein X is CR^{a}R^{b}, NR^{c}, O, S, or SiR^{d}R^{e};
R^{a}, R^{b}, R^{c}, R^{d}, and R^{e} are each independently hydrogen, deuterium, C1-C60 alkyl, or C6-C60 aryl;
L₁ to L₅ are each independently a single bond, C6-C60 arylene, or C3-C60 heteroarylene, and the arylene and heteroarylene of L₁ to L₅ may be further substituted by one or more selected from the group consisting of C1-C60 alkyl, C6-C30 aryl, and —NR′R″;
R′ and R″ are each independently C6-C60 aryl or C3-C60 heteroaryl;
R₁ to R₁₀ are each independently C6-C60 aryl, C3-C60 heteroaryl, or -L₁₁-R₁₁;
L₁₁ is C6-C60 arylene or C3-C60 heteroarylene;
R₁₁ is C6-C60 aryl, C3-C60 heteroaryl, or —NR₁₂R₁₃;
R¹² and R¹³ are each independently C6-C60 aryl or C3-C60 heteroaryl;
the aryl and heteroaryl of R₁ to R₁₀, the arylene and heteroarylene of L₁₁, the aryl and heteroaryl of R₁, and the aryl and heteroaryl of R₁₂ and R₁₃ may be further substituted by one or more selected from the group consisting of C1-C60 alkyl, deuterium, C6-C60 aryl, C6-C60 aryl C1-C60 alkyl, C1-C60 alkyl C6-C60 aryl, and C3-C60 heteroaryl; and
p, q, r, s, and t are each independently an integer of 0 to 2, and satisfy 1≤p+q+r+s+t≤10.

3. The spirofluorene compound of claim 2, wherein the spirofluorene compound is represented by any one of the following Chemical Formulas 2 to 6: 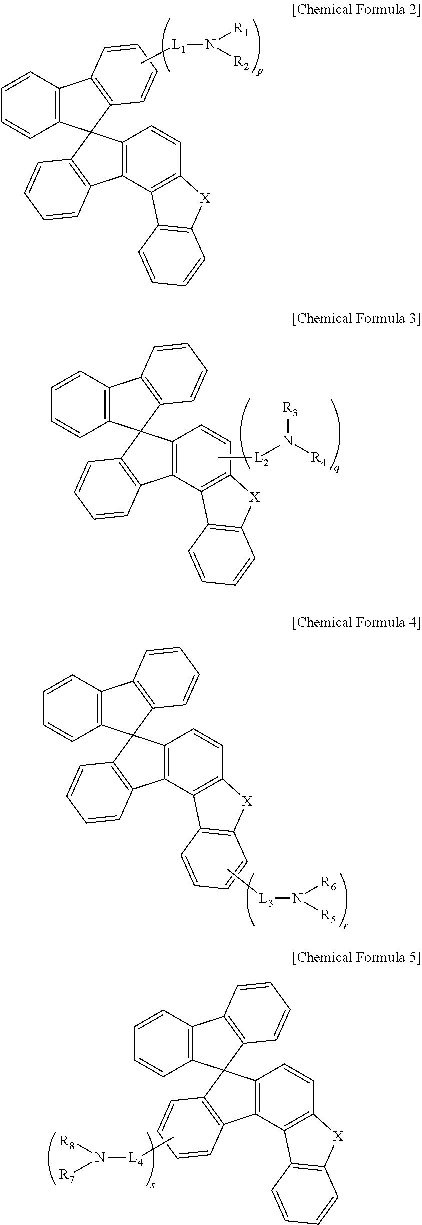 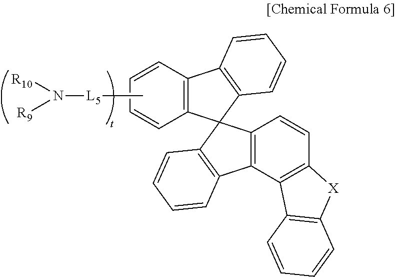
in Chemical Formulas 2 to 6,
X is CR^{a}R^{b}, NR^{c}, O, S, or SiR^{d}R^{e};
R^{a}, R^{b}, R^{c}, R^{d}, and R^{e} are each independently C1-C30 alkyl or C6-C30 aryl;
L₁ to L₅ are each independently a single bond, C6-C30 arylene, or C3-C30 heteroarylene, and the arylene and heteroarylene of L₁ to L₅ may be further substituted by one or more selected from the group consisting of C1-C30 alkyl, C6-C30 aryl, and —NR′R″;
R′ and R″ are each independently C6-C30 aryl or C3-C30 heteroaryl;
R₁ to R₁₀ are each independently C6-C30 aryl, C3-C30 heteroaryl, or -L₁₁-R₁₁;
L₁₁ is C6-C30 arylene or C3-C30 heteroarylene;
R₁₁ is C6-C30 aryl, C3-C30 heteroaryl, or —NR₁₂R₁₃;
R₁₂ and R₁₃ are each independently C6-C30 aryl or C3-C30 heteroaryl;
the aryl and heteroaryl of R₁ to R₁₀, the arylene and heteroarylene of L₁₁, the aryl and heteroaryl of R₁, and the aryl and heteroaryl of R¹² and R¹³ may be further substituted by one or more selected from the group consisting of C1-C30 alkyl, C6-C30 aryl, C6-C30 aryl C1-C30 alkyl, C1-C30 alkyl C6-C30 aryl, and C3-C30 heteroaryl; and
p, q, r, s, and t are each independently an integer of 1 or 2.

4. The spirofluorene compound of claim 3, wherein the spirofluorene compound is represented by any one of the following Chemical Formulas 7 to 13: 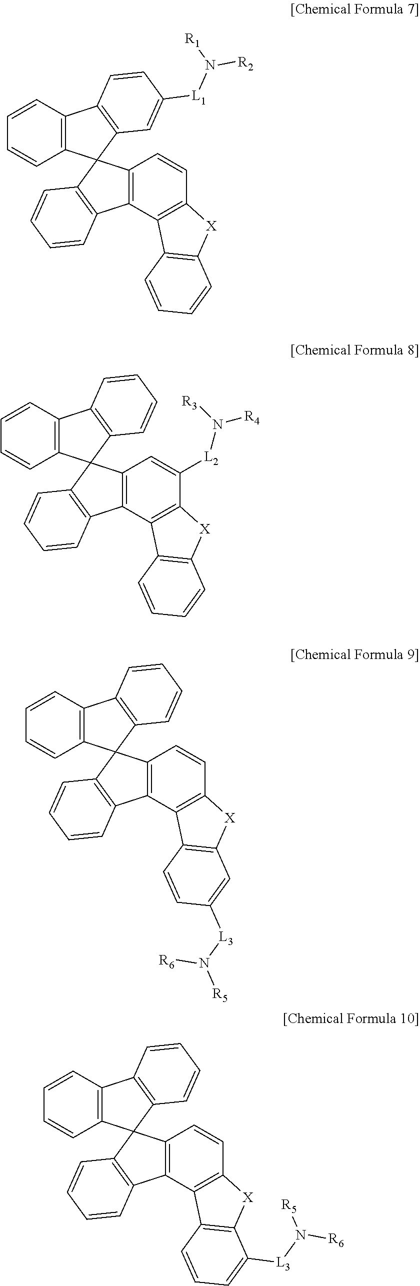 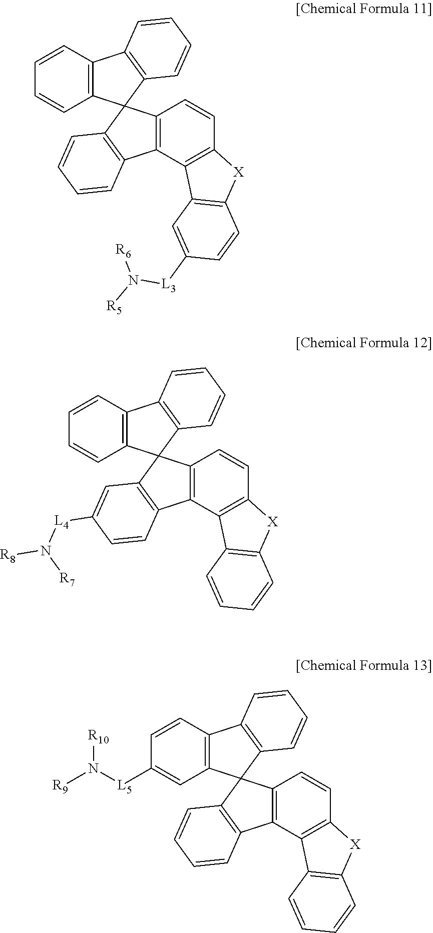
in Chemical Formulas 7 to 13,
X is CR^{a}R^{b}, NR^{c}, or O;
R^{a} and R^{b} are each independently C1-C20 alkyl;
R^{c} is C6-C20 aryl;
L₁ to L₅ are each independently a single bond, C6-C20 arylene, or C3-C20 heteroarylene, and the arylene and heteroarylene of L₁ to L₅ may be further substituted by one or more selected from the group consisting of C1-C20 alkyl, C6-C20 aryl, and —NR′R″;
R′ and R″ are each independently C6-C20 aryl or C3-C20 heteroaryl;
R₁ to R₁₀ are each independently C6-C20 aryl, C3-C20 heteroaryl, or -L₁₁-R₁₁;
L₁₁ is C6-C20 arylene or C3-C20 heteroarylene;
R₁₁ is C6-C20 aryl, C3-C20 heteroaryl, or —NR₁₂R₁₃;
R₁₂ and R₁₃ are each independently C6-C20 aryl or C3-C20 heteroaryl; and
the aryl and heteroaryl of R₁ to R₁₀, the arylene and heteroarylene of L₁₁, the aryl and heteroaryl of R₁₁ and the aryl and heteroaryl of R₁₂ and R₁₃ may be further substituted by one or more selected from the group consisting of C1-C20 alkyl, C6-C20 aryl, C6-C20 aryl C1-C20 alkyl, C1-C20 alkyl C6-C20 aryl, and C3-C20 heteroaryl.

5. The spirofluorene compound of claim 4, wherein L₁ to L₅ are each independently a single bond or selected from the following structures: 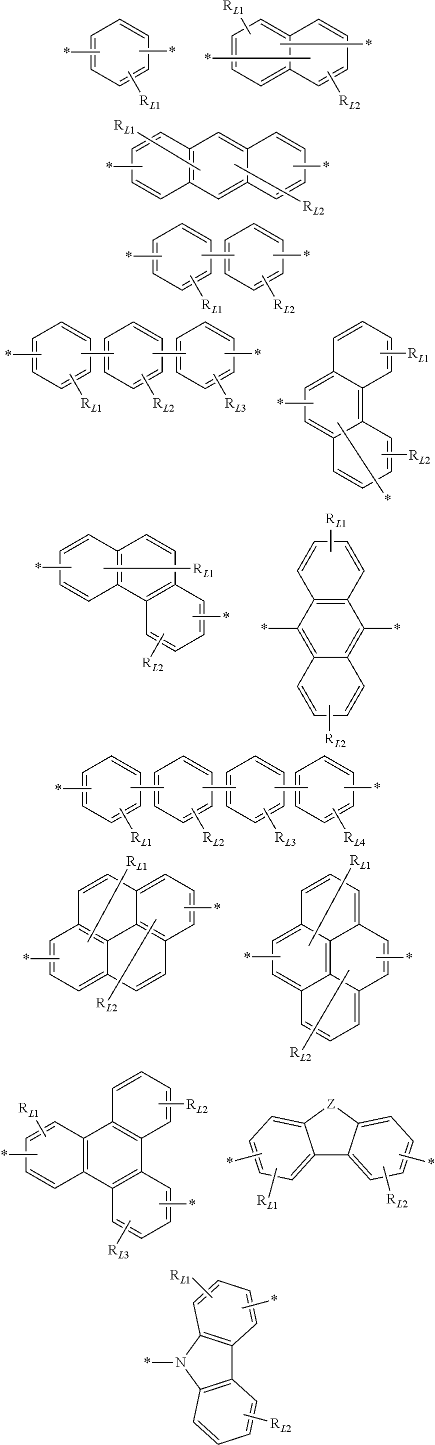
wherein
R_{L1}, R_{L2}, R_{L3}, and R_{L4} are each independently hydrogen, C6-C20 aryl, or NR′R″;
R′ and R″ are each independently C6-C20 aryl or C3-C20 heteroaryl;
Z is CR_{Z1}R_{Z2}, NR_{Z3}, O, or S;
R_{Z1} and R_{Z2} are each independently C1-C20 alkyl or C6-C20 aryl; and
R_{Z3} is C6-C20 aryl.

6. The spirofluorene compound of claim 4, wherein R₁ to R₁₀ are each independently selected from the following structures: 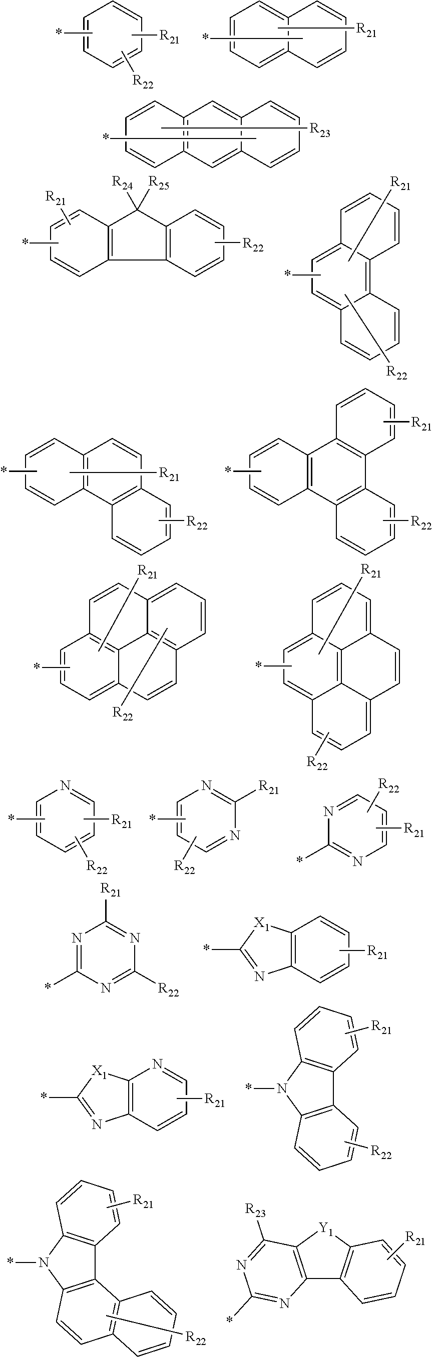 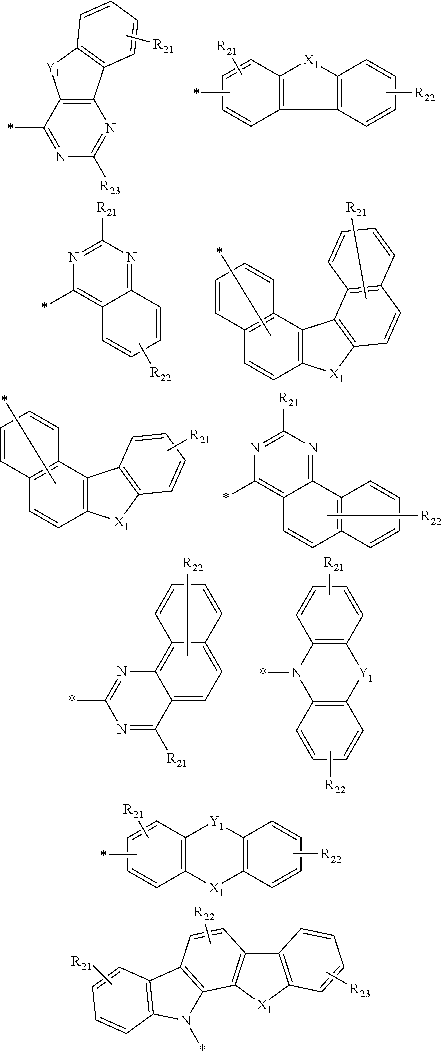
wherein
X₁ is NR₃₁, O, or S;
Y₁ is CR₃₂R₃₃, O, or S;
R₃₁ is C6-C20 aryl or C3-C20 heteroaryl;
R₃₂ and R₃₃ are each independently C1-C20 alkyl, C6-C20 aryl, or C3-C20 heteroaryl;
R₂₁, R₂₂, and R₂₃ are each independently hydrogen, C6-C20 aryl, C3-C20 heteroaryl, or —NR₁₂R₁₃;
R₁₂ and R₁₃ are each independently C6-C20 aryl or C3-C20 heteroaryl;
R₂₄ and R₂₅ are each independently C1-C20 alkyl or C6-C20 aryl; and
the aryl and heteroaryl of R₂₁, R₂₂, and R₂₃ may be further substituted by one or more selected from the group consisting of C1-C20 alkyl and C6-C20 aryl.

7. The spirofluorene compound of claim 1, wherein the spirofluorene compound is selected from the following compounds:

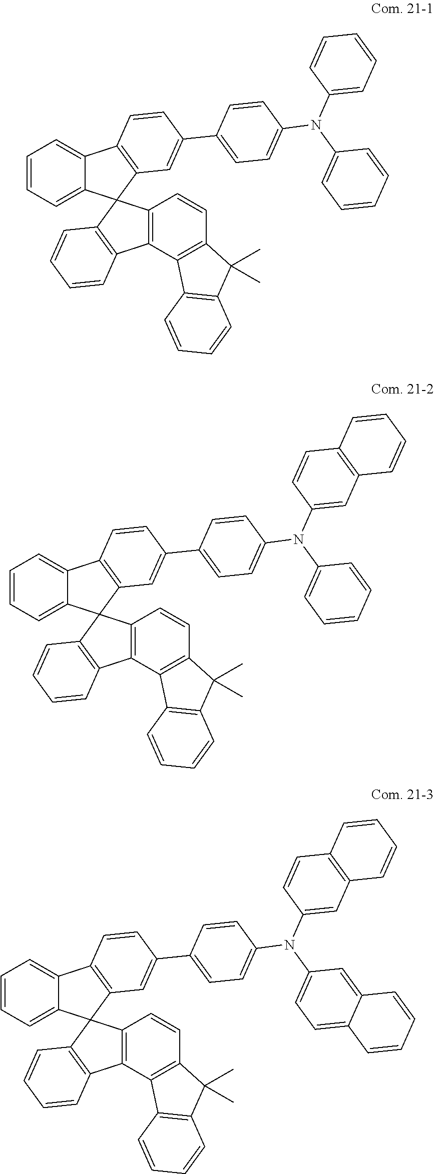 [SMILES: CC1(C)c2ccccc2-c2c1ccc1c2-c2ccccc2C12c1ccccc1-c1ccc(-c3ccc(N(c4ccccc4)c4ccccc4)cc3)cc12 ; CC1(C)c2ccccc2-c2c1ccc1c2-c2ccccc2C12c1ccccc1-c1ccc(-c3ccc(N(c4ccccc4)c4ccc5ccccc5c4)cc3)cc12 ; CC1(C)c2ccccc2-c2c1ccc1c2-c2ccccc2C12c1ccccc1-c1ccc(-c3ccc(N(c4ccc5ccccc5c4)c4ccc5ccccc5c4)cc3)cc12]

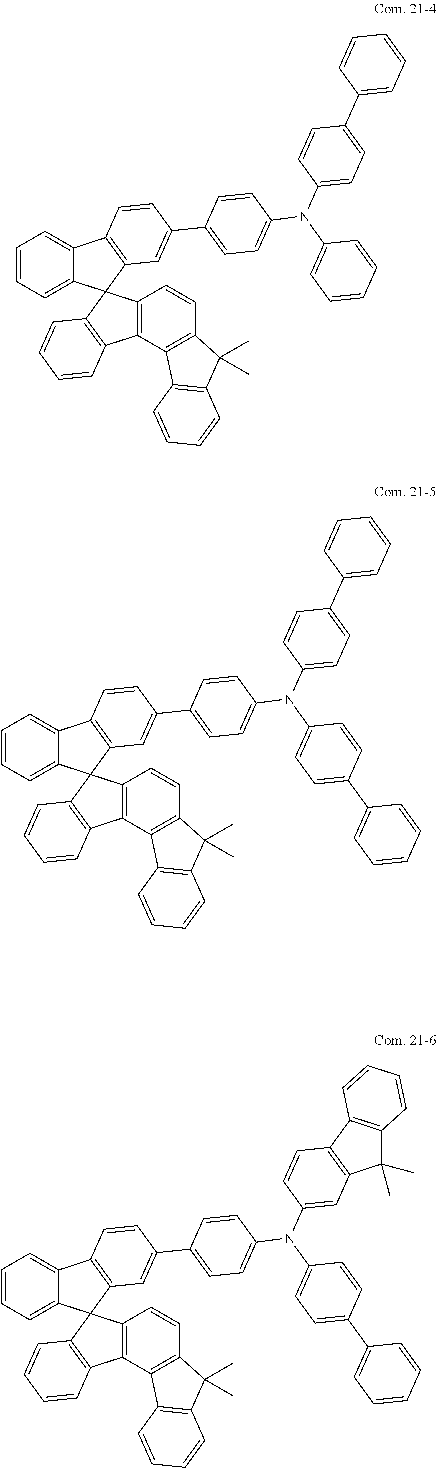 [SMILES: CC1(C)c2ccccc2-c2c1ccc1c2-c2ccccc2C12c1ccccc1-c1ccc(-c3ccc(N(c4ccccc4)c4ccc(-c5ccccc5)cc4)cc3)cc12 ; CC1(C)c2ccccc2-c2c1ccc1c2-c2ccccc2C12c1ccccc1-c1ccc(-c3ccc(N(c4ccc(-c5ccccc5)cc4)c4ccc(-c5ccccc5)cc4)cc3)cc12 ; CC1(C)c2ccccc2-c2ccc(N(c3ccc(-c4ccccc4)cc3)c3ccc(-c4ccc5c(c4)C4(c6ccccc6-5)c5ccccc5-c5c4ccc4c5-c5ccccc5C4(C)C)cc3)cc21]

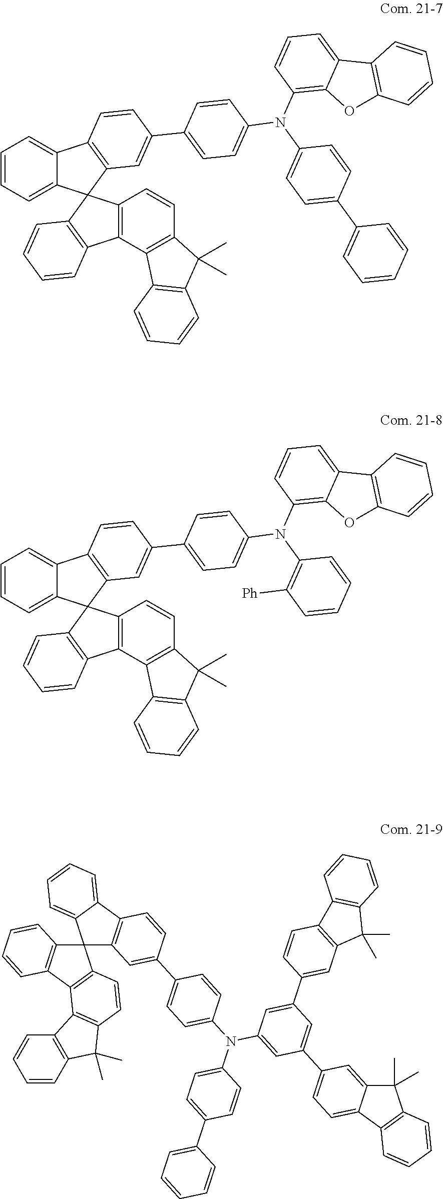 [SMILES: CC1(C)c2ccccc2-c2c1ccc1c2-c2ccccc2C12c1ccccc1-c1ccc(-c3ccc(N(c4ccc(-c5ccccc5)cc4)c4cccc5c4oc4ccccc45)cc3)cc12 ; CC1(C)c2ccccc2-c2c1ccc1c2-c2ccccc2C12c1ccccc1-c1ccc(-c3ccc(N(c4ccccc4-c4ccccc4)c4cccc5c4oc4ccccc45)cc3)cc12 ; CC1(C)c2ccccc2-c2ccc(-c3cc(-c4ccc5c(c4)C(C)(C)c4ccccc4-5)cc(N(c4ccc(-c5ccccc5)cc4)c4ccc(-c5ccc6c(c5)C5(c7ccccc7-6)c6ccccc6-c6c5ccc5c6-c6ccccc6C5(C)C)cc4)c3)cc21]

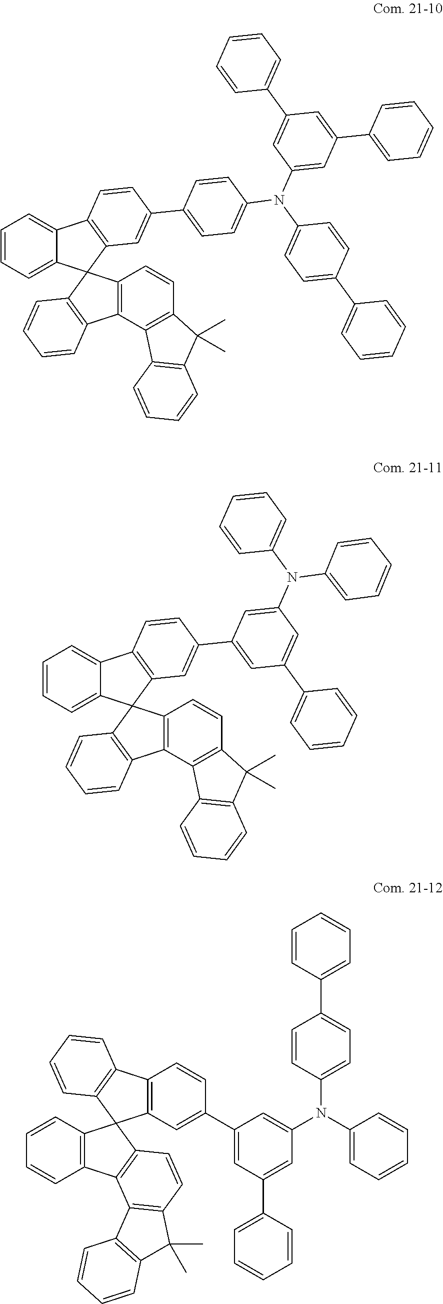 [SMILES: CC1(C)c2ccccc2-c2c1ccc1c2-c2ccccc2C12c1ccccc1-c1ccc(-c3ccc(N(c4ccc(-c5ccccc5)cc4)c4cc(-c5ccccc5)cc(-c5ccccc5)c4)cc3)cc12 ; CC1(C)c2ccccc2-c2c1ccc1c2-c2ccccc2C12c1ccccc1-c1ccc(-c3cc(-c4ccccc4)cc(N(c4ccccc4)c4ccccc4)c3)cc12 ; CC1(C)c2ccccc2-c2c1ccc1c2-c2ccccc2C12c1ccccc1-c1ccc(-c3cc(-c4ccccc4)cc(N(c4ccccc4)c4ccc(-c5ccccc5)cc4)c3)cc12]

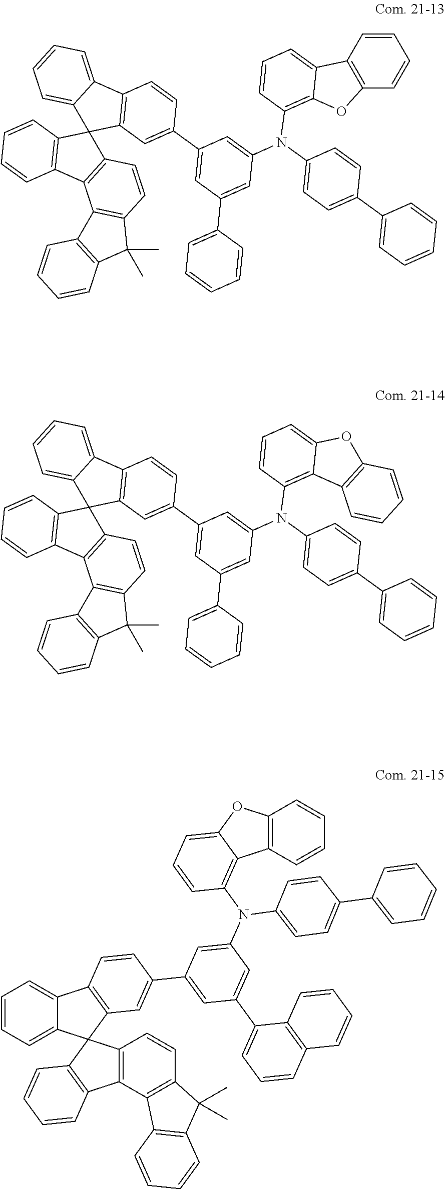 [SMILES: CC1(C)c2ccccc2-c2c1ccc1c2-c2ccccc2C12c1ccccc1-c1ccc(-c3cc(-c4ccccc4)cc(N(c4ccc(-c5ccccc5)cc4)c4cccc5c4oc4ccccc45)c3)cc12 ; CC1(C)c2ccccc2-c2c1ccc1c2-c2ccccc2C12c1ccccc1-c1ccc(-c3cc(-c4ccccc4)cc(N(c4ccc(-c5ccccc5)cc4)c4cccc5oc6ccccc6c45)c3)cc12 ; CC1(C)c2ccccc2-c2c1ccc1c2-c2ccccc2C12c1ccccc1-c1ccc(-c3cc(-c4cccc5ccccc45)cc(N(c4ccc(-c5ccccc5)cc4)c4cccc5oc6ccccc6c45)c3)cc12]

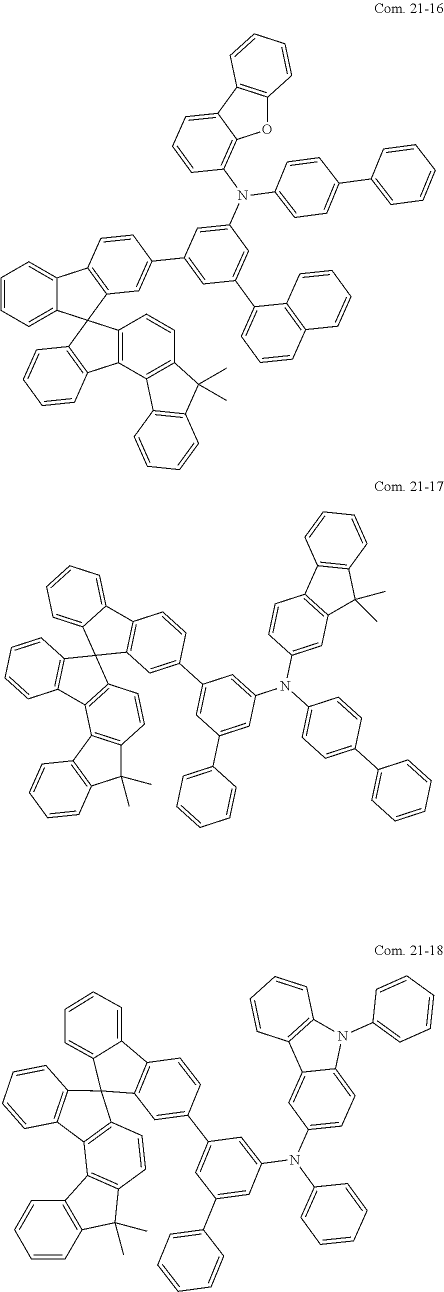 [SMILES: CC1(C)c2ccccc2-c2c1ccc1c2-c2ccccc2C12c1ccccc1-c1ccc(-c3cc(-c4cccc5ccccc45)cc(N(c4ccc(-c5ccccc5)cc4)c4cccc5c4oc4ccccc45)c3)cc12 ; CC1(C)c2ccccc2-c2ccc(N(c3ccc(-c4ccccc4)cc3)c3cc(-c4ccccc4)cc(-c4ccc5c(c4)C4(c6ccccc6-5)c5ccccc5-c5c4ccc4c5-c5ccccc5C4(C)C)c3)cc21 ; CC1(C)c2ccccc2-c2c1ccc1c2-c2ccccc2C12c1ccccc1-c1ccc(-c3cc(-c4ccccc4)cc(N(c4ccccc4)c4ccc5c(c4)c4ccccc4n5-c4ccccc4)c3)cc12]

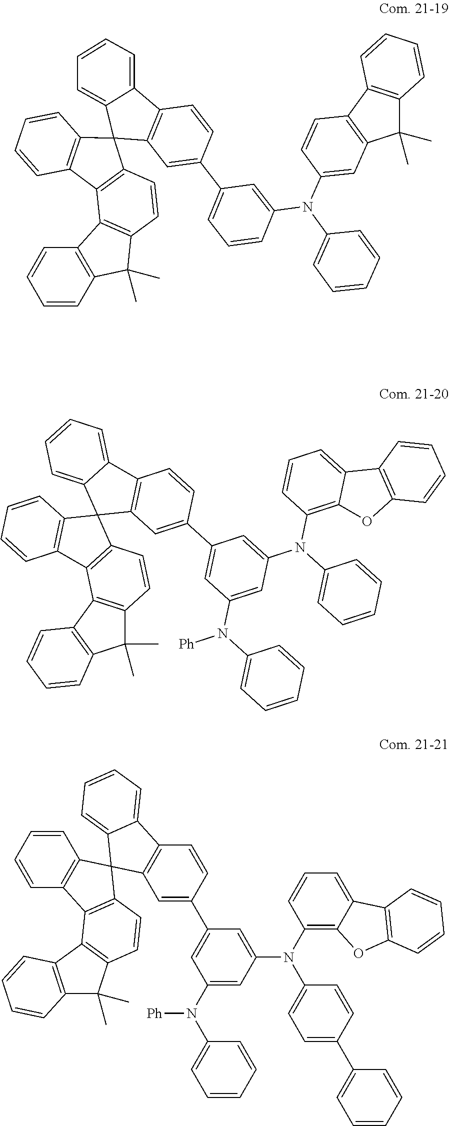 [SMILES: CC1(C)c2ccccc2-c2ccc(N(c3ccccc3)c3cccc(-c4ccc5c(c4)C4(c6ccccc6-5)c5ccccc5-c5c4ccc4c5-c5ccccc5C4(C)C)c3)cc21 ; CC1(C)c2ccccc2-c2c1ccc1c2-c2ccccc2C12c1ccccc1-c1ccc(-c3cc(N(c4ccccc4)c4ccccc4)cc(N(c4ccccc4)c4cccc5c4oc4ccccc45)c3)cc12 ; CC1(C)c2ccccc2-c2c1ccc1c2-c2ccccc2C12c1ccccc1-c1ccc(-c3cc(N(c4ccccc4)c4ccccc4)cc(N(c4ccc(-c5ccccc5)cc4)c4cccc5c4oc4ccccc45)c3)cc12]

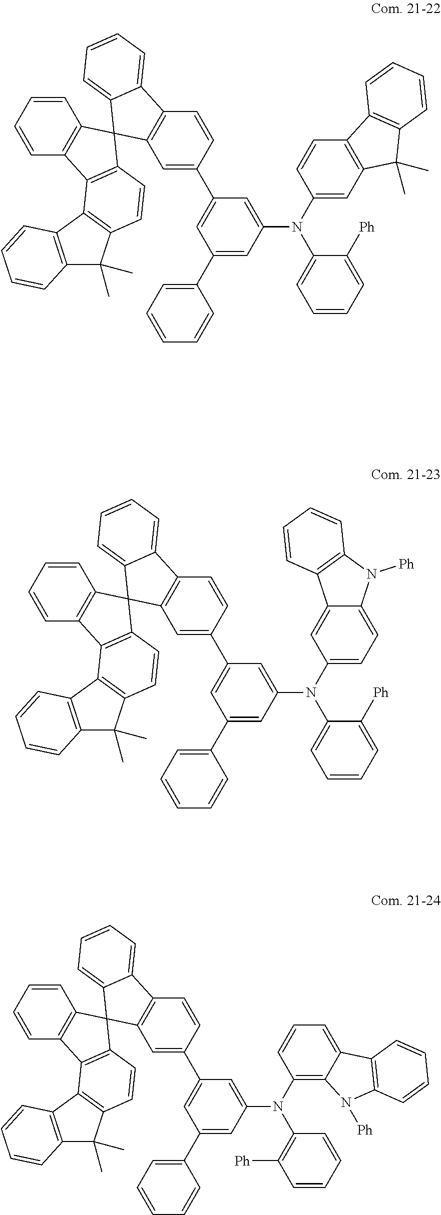 [SMILES: CC1(C)c2ccccc2-c2ccc(N(c3cc(-c4ccccc4)cc(-c4ccc5c(c4)C4(c6ccccc6-5)c5ccccc5-c5c4ccc4c5-c5ccccc5C4(C)C)c3)c3ccccc3-c3ccccc3)cc21 ; CC1(C)c2ccccc2-c2c1ccc1c2-c2ccccc2C12c1ccccc1-c1ccc(-c3cc(-c4ccccc4)cc(N(c4ccc5c(c4)c4ccccc4n5-c4ccccc4)c4ccccc4-c4ccccc4)c3)cc12 ; CC1(C)c2ccccc2-c2c1ccc1c2-c2ccccc2C12c1ccccc1-c1ccc(-c3cc(-c4ccccc4)cc(N(c4ccccc4-c4ccccc4)c4cccc5c6ccccc6n(-c6ccccc6)c45)c3)cc12]

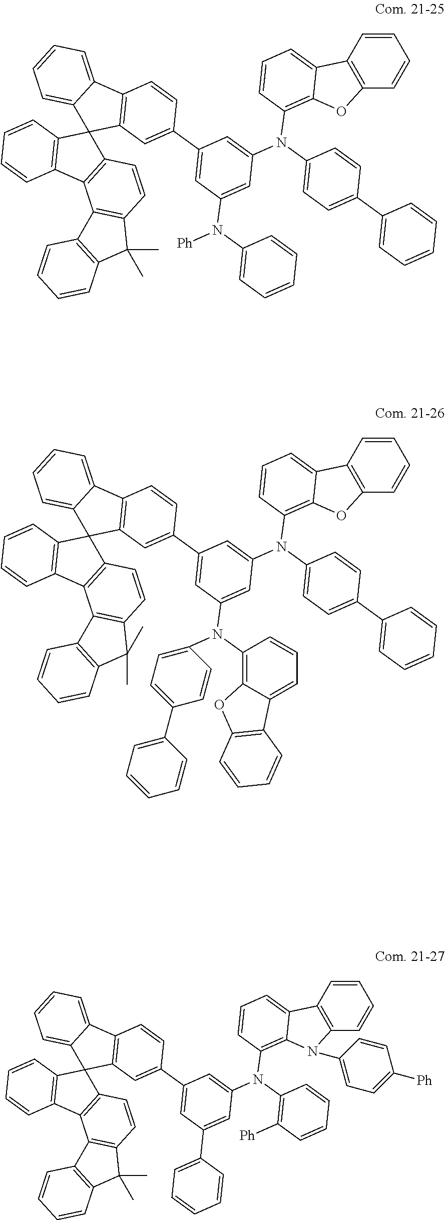 [SMILES: CC1(C)c2ccccc2-c2c1ccc1c2-c2ccccc2C12c1ccccc1-c1ccc(-c3cc(N(c4ccccc4)c4ccccc4)cc(N(c4ccc(-c5ccccc5)cc4)c4cccc5c4oc4ccccc45)c3)cc12 ; CC1(C)c2ccccc2-c2c1ccc1c2-c2ccccc2C12c1ccccc1-c1ccc(-c3cc(N(c4ccc(-c5ccccc5)cc4)c4cccc5c4oc4ccccc45)cc(N(c4ccc(-c5ccccc5)cc4)c4cccc5c4oc4ccccc45)c3)cc12 ; CC1(C)c2ccccc2-c2c1ccc1c2-c2ccccc2C12c1ccccc1-c1ccc(-c3cc(-c4ccccc4)cc(N(c4ccccc4-c4ccccc4)c4cccc5c6ccccc6n(-c6ccc(-c7ccccc7)cc6)c45)c3)cc12]

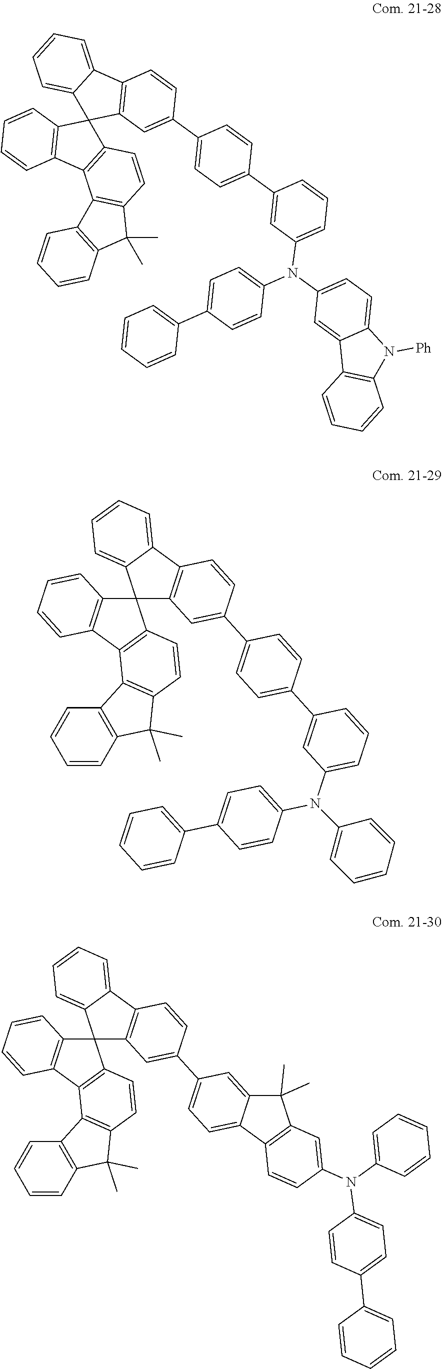 [SMILES: CC1(C)c2ccccc2-c2c1ccc1c2-c2ccccc2C12c1ccccc1-c1ccc(-c3ccc(-c4cccc(N(c5ccc(-c6ccccc6)cc5)c5ccc6c(c5)c5ccccc5n6-c5ccccc5)c4)cc3)cc12 ; CC1(C)c2ccccc2-c2c1ccc1c2-c2ccccc2C12c1ccccc1-c1ccc(-c3ccc(-c4cccc(N(c5ccccc5)c5ccc(-c6ccccc6)cc5)c4)cc3)cc12 ; CC1(C)c2cc(-c3ccc4c(c3)C3(c5ccccc5-4)c4ccccc4-c4c3ccc3c4-c4ccccc4C3(C)C)ccc2-c2ccc(N(c3ccccc3)c3ccc(-c4ccccc4)cc3)cc21]

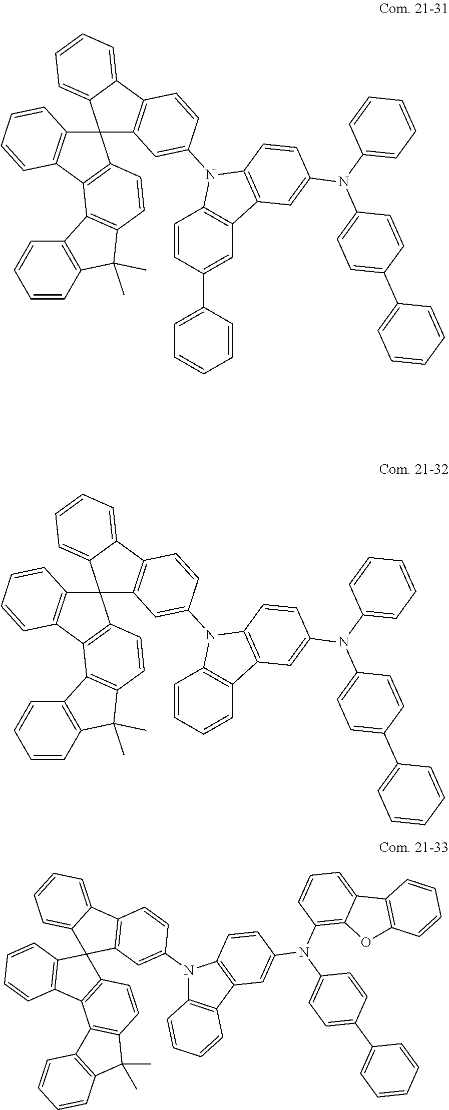 [SMILES: CC1(C)c2ccccc2-c2c1ccc1c2-c2ccccc2C12c1ccccc1-c1ccc(-n3c4ccc(-c5ccccc5)cc4c4cc(N(c5ccccc5)c5ccc(-c6ccccc6)cc5)ccc43)cc12 ; CC1(C)c2ccccc2-c2c1ccc1c2-c2ccccc2C12c1ccccc1-c1ccc(-n3c4ccccc4c4cc(N(c5ccccc5)c5ccc(-c6ccccc6)cc5)ccc43)cc12 ; CC1(C)c2ccccc2-c2c1ccc1c2-c2ccccc2C12c1ccccc1-c1ccc(-n3c4ccccc4c4cc(N(c5ccc(-c6ccccc6)cc5)c5cccc6c5oc5ccccc56)ccc43)cc12]

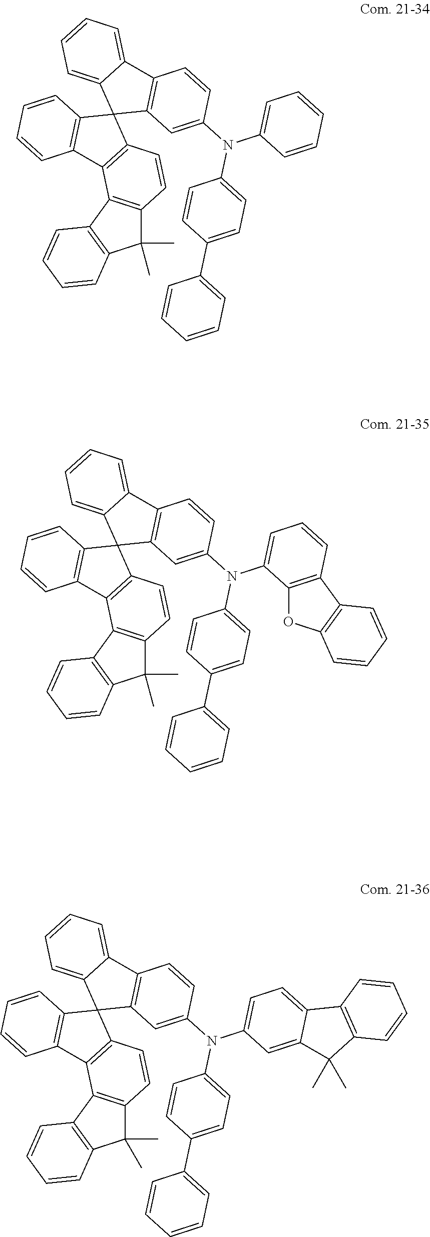 [SMILES: CC1(C)c2ccccc2-c2c1ccc1c2-c2ccccc2C12c1ccccc1-c1ccc(N(c3ccccc3)c3ccc(-c4ccccc4)cc3)cc12 ; CC1(C)c2ccccc2-c2c1ccc1c2-c2ccccc2C12c1ccccc1-c1ccc(N(c3ccc(-c4ccccc4)cc3)c3cccc4c3oc3ccccc34)cc12 ; CC1(C)c2ccccc2-c2ccc(N(c3ccc(-c4ccccc4)cc3)c3ccc4c(c3)C3(c5ccccc5-4)c4ccccc4-c4c3ccc3c4-c4ccccc4C3(C)C)cc21]

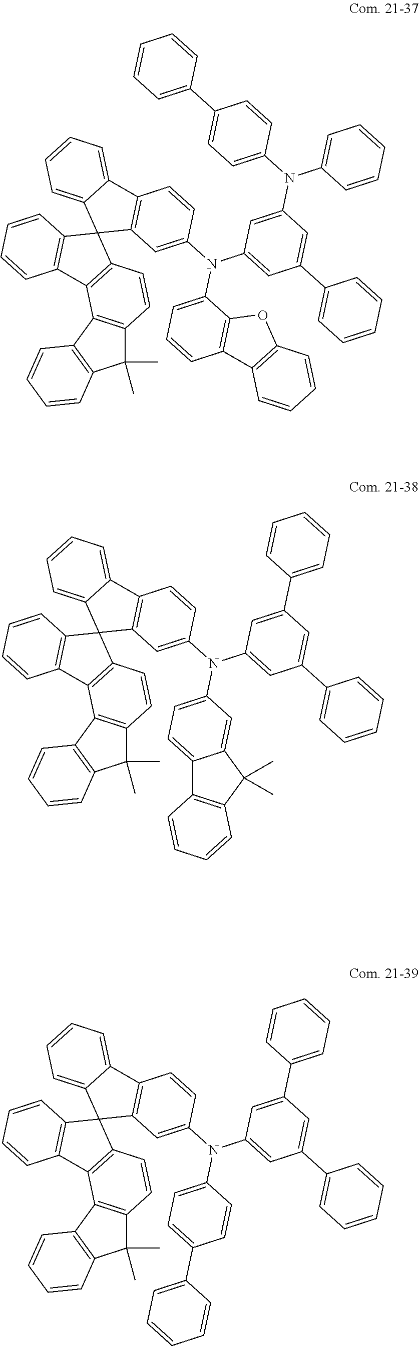 [SMILES: CC1(C)c2ccccc2-c2c1ccc1c2-c2ccccc2C12c1ccccc1-c1ccc(N(c3cc(-c4ccccc4)cc(N(c4ccccc4)c4ccc(-c5ccccc5)cc4)c3)c3cccc4c3oc3ccccc34)cc12 ; CC1(C)c2ccccc2-c2ccc(N(c3cc(-c4ccccc4)cc(-c4ccccc4)c3)c3ccc4c(c3)C3(c5ccccc5-4)c4ccccc4-c4c3ccc3c4-c4ccccc4C3(C)C)cc21 ; CC1(C)c2ccccc2-c2c1ccc1c2-c2ccccc2C12c1ccccc1-c1ccc(N(c3ccc(-c4ccccc4)cc3)c3cc(-c4ccccc4)cc(-c4ccccc4)c3)cc12]

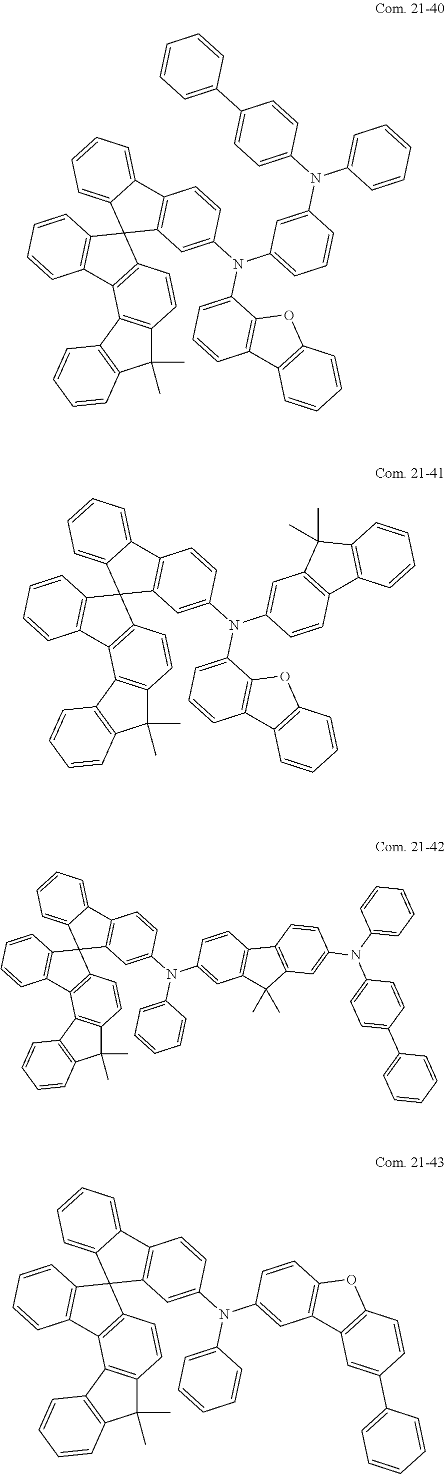 [SMILES: CC1(C)c2ccccc2-c2c1ccc1c2-c2ccccc2C12c1ccccc1-c1ccc(N(c3cccc(N(c4ccccc4)c4ccc(-c5ccccc5)cc4)c3)c3cccc4c3oc3ccccc34)cc12 ; CC1(C)c2ccccc2-c2ccc(N(c3ccc4c(c3)C3(c5ccccc5-4)c4ccccc4-c4c3ccc3c4-c4ccccc4C3(C)C)c3cccc4c3oc3ccccc34)cc21 ; CC1(C)c2cc(N(c3ccccc3)c3ccc(-c4ccccc4)cc3)ccc2-c2ccc(N(c3ccccc3)c3ccc4c(c3)C3(c5ccccc5-4)c4ccccc4-c4c3ccc3c4-c4ccccc4C3(C)C)cc21 ; CC1(C)c2ccccc2-c2c1ccc1c2-c2ccccc2C12c1ccccc1-c1ccc(N(c3ccccc3)c3ccc4oc5ccc(-c6ccccc6)cc5c4c3)cc12]

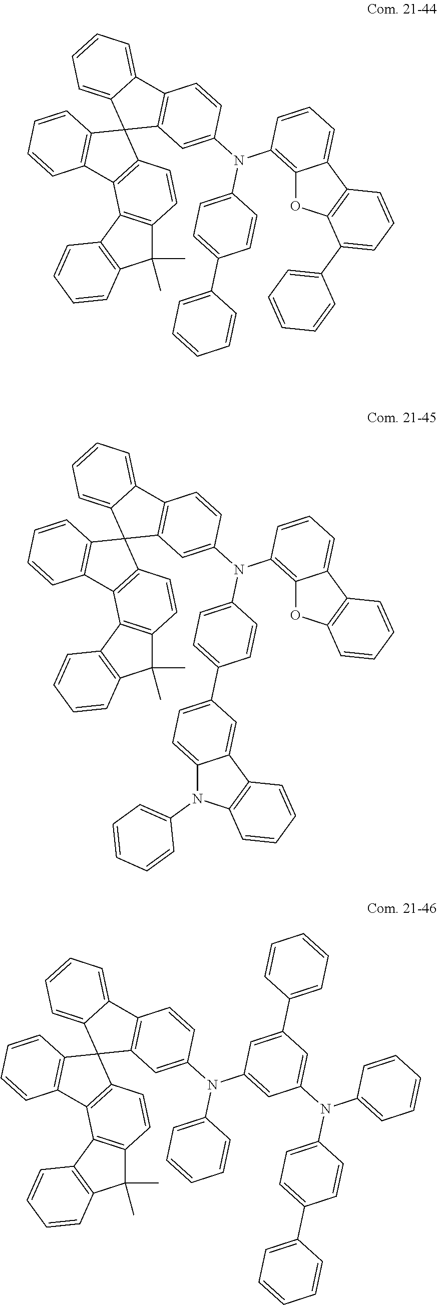 [SMILES: CC1(C)c2ccccc2-c2c1ccc1c2-c2ccccc2C12c1ccccc1-c1ccc(N(c3ccc(-c4ccccc4)cc3)c3cccc4c3oc3c(-c5ccccc5)cccc34)cc12 ; CC1(C)c2ccccc2-c2c1ccc1c2-c2ccccc2C12c1ccccc1-c1ccc(N(c3ccc(-c4ccc5c(c4)c4ccccc4n5-c4ccccc4)cc3)c3cccc4c3oc3ccccc34)cc12 ; CC1(C)c2ccccc2-c2c1ccc1c2-c2ccccc2C12c1ccccc1-c1ccc(N(c3ccccc3)c3cc(-c4ccccc4)cc(N(c4ccccc4)c4ccc(-c5ccccc5)cc4)c3)cc12]

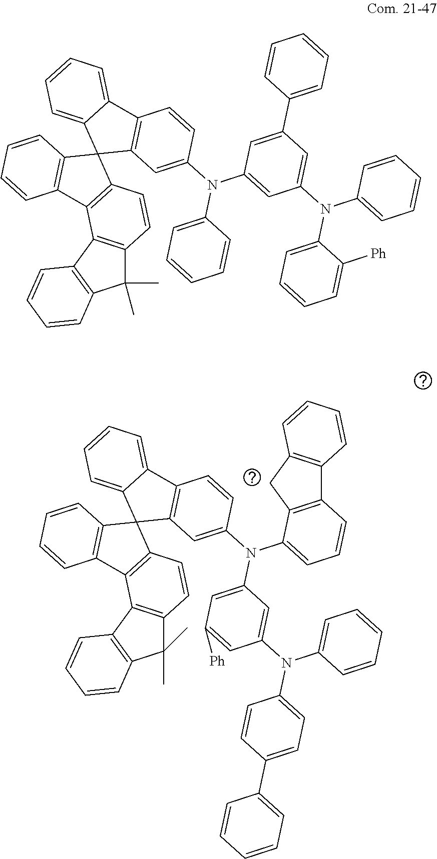 [SMILES: CC1(C)c2ccccc2-c2c1ccc1c2-c2ccccc2C12c1ccccc1-c1ccc(N(c3ccccc3)c3cc(-c4ccccc4)cc(N(c4ccccc4)c4ccccc4-c4ccccc4)c3)cc12 ; CC1(C)c2ccccc2-c2c1ccc1c2-c2ccccc2C12c1ccccc1-c1ccc(N(c3cc(-c4ccccc4)cc(N(c4ccccc4)c4ccc(-c5ccccc5)cc4)c3)c3cccc4c3Cc3ccccc3-4)cc12]

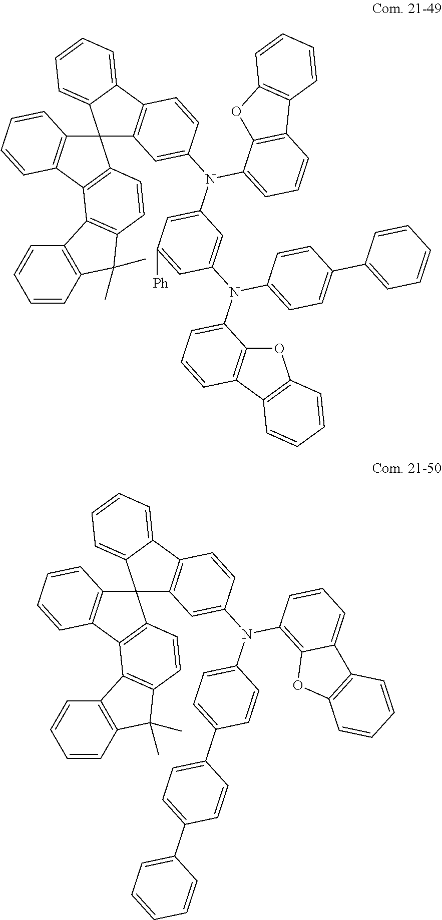 [SMILES: CC1(C)c2ccccc2-c2c1ccc1c2-c2ccccc2C12c1ccccc1-c1ccc(N(c3cc(-c4ccccc4)cc(N(c4ccc(-c5ccccc5)cc4)c4cccc5c4oc4ccccc45)c3)c3cccc4c3oc3ccccc34)cc12 ; CC1(C)c2ccccc2-c2c1ccc1c2-c2ccccc2C12c1ccccc1-c1ccc(N(c3ccc(-c4ccc(-c5ccccc5)cc4)cc3)c3cccc4c3oc3ccccc34)cc12]

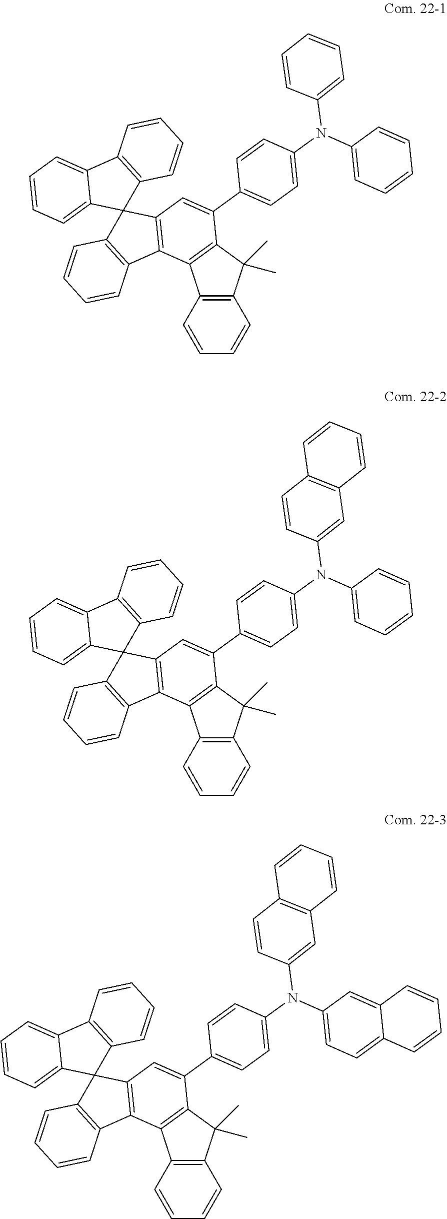 [SMILES: CC1(C)c2ccccc2-c2c3c(cc(-c4ccc(N(c5ccccc5)c5ccccc5)cc4)c21)C1(c2ccccc2-c2ccccc21)c1ccccc1-3 ; CC1(C)c2ccccc2-c2c3c(cc(-c4ccc(N(c5ccccc5)c5ccc6ccccc6c5)cc4)c21)C1(c2ccccc2-c2ccccc21)c1ccccc1-3 ; CC1(C)c2ccccc2-c2c3c(cc(-c4ccc(N(c5ccc6ccccc6c5)c5ccc6ccccc6c5)cc4)c21)C1(c2ccccc2-c2ccccc21)c1ccccc1-3]

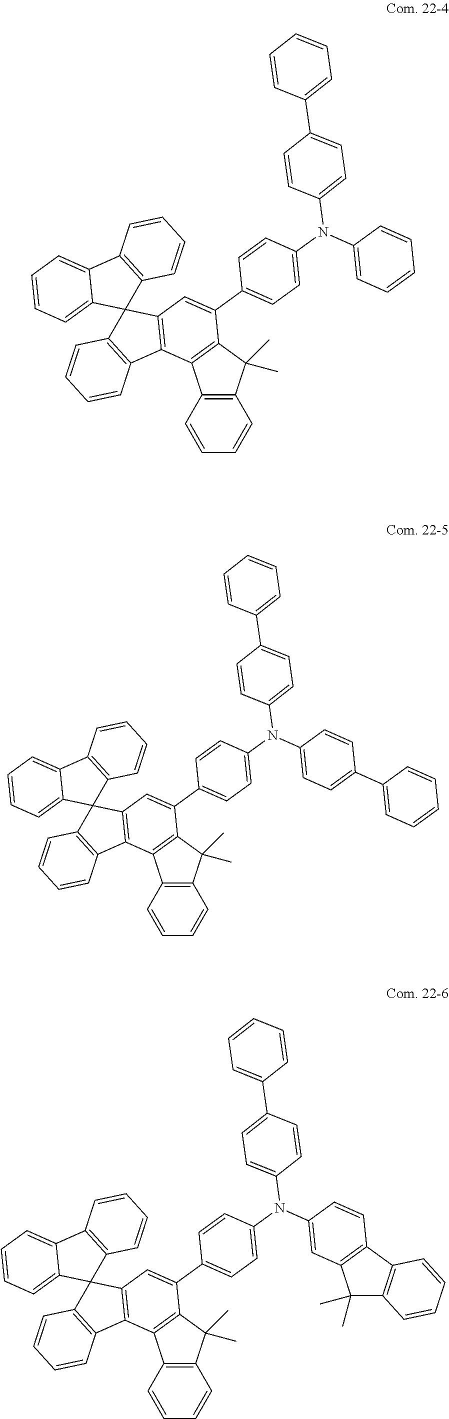 [SMILES: CC1(C)c2ccccc2-c2c3c(cc(-c4ccc(N(c5ccccc5)c5ccc(-c6ccccc6)cc5)cc4)c21)C1(c2ccccc2-c2ccccc21)c1ccccc1-3 ; CC1(C)c2ccccc2-c2c3c(cc(-c4ccc(N(c5ccc(-c6ccccc6)cc5)c5ccc(-c6ccccc6)cc5)cc4)c21)C1(c2ccccc2-c2ccccc21)c1ccccc1-3 ; CC1(C)c2ccccc2-c2ccc(N(c3ccc(-c4ccccc4)cc3)c3ccc(-c4cc5c(c6c4C(C)(C)c4ccccc4-6)-c4ccccc4C54c5ccccc5-c5ccccc54)cc3)cc21]

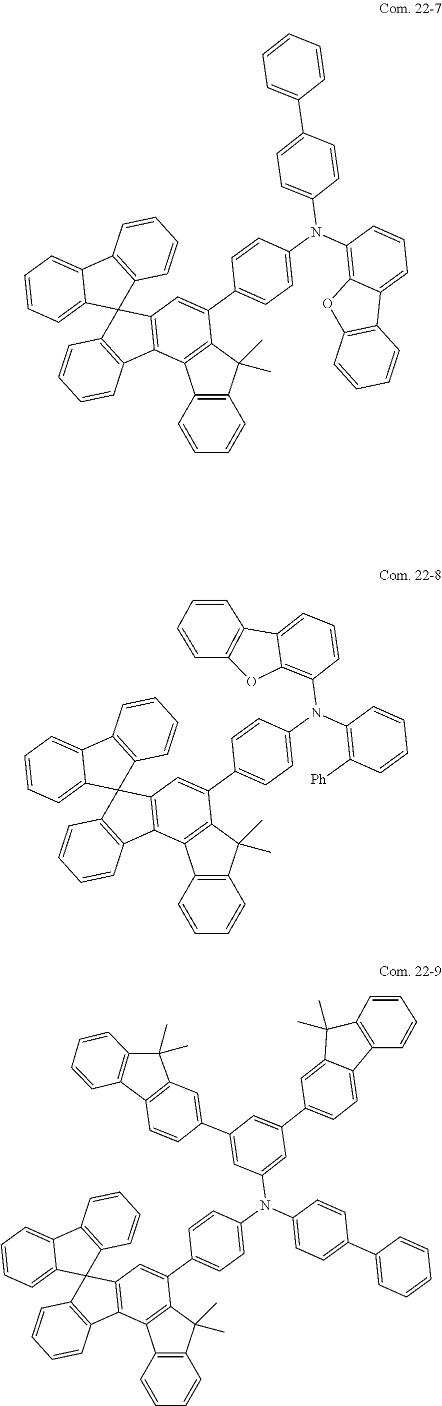 [SMILES: CC1(C)c2ccccc2-c2c3c(cc(-c4ccc(N(c5ccc(-c6ccccc6)cc5)c5cccc6c5oc5ccccc56)cc4)c21)C1(c2ccccc2-c2ccccc21)c1ccccc1-3 ; CC1(C)c2ccccc2-c2c3c(cc(-c4ccc(N(c5ccccc5-c5ccccc5)c5cccc6c5oc5ccccc56)cc4)c21)C1(c2ccccc2-c2ccccc21)c1ccccc1-3 ; CC1(C)c2ccccc2-c2ccc(-c3cc(-c4ccc5c(c4)C(C)(C)c4ccccc4-5)cc(N(c4ccc(-c5ccccc5)cc4)c4ccc(-c5cc6c(c7c5C(C)(C)c5ccccc5-7)-c5ccccc5C65c6ccccc6-c6ccccc65)cc4)c3)cc21]

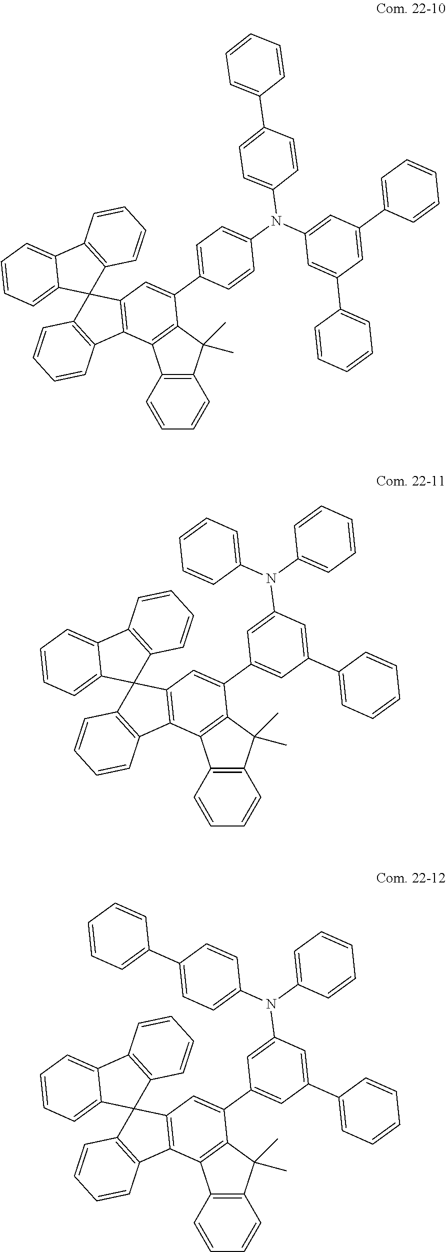 [SMILES: CC1(C)c2ccccc2-c2c3c(cc(-c4ccc(N(c5ccc(-c6ccccc6)cc5)c5cc(-c6ccccc6)cc(-c6ccccc6)c5)cc4)c21)C1(c2ccccc2-c2ccccc21)c1ccccc1-3 ; CC1(C)c2ccccc2-c2c3c(cc(-c4cc(-c5ccccc5)cc(N(c5ccccc5)c5ccccc5)c4)c21)C1(c2ccccc2-c2ccccc21)c1ccccc1-3 ; CC1(C)c2ccccc2-c2c3c(cc(-c4cc(-c5ccccc5)cc(N(c5ccccc5)c5ccc(-c6ccccc6)cc5)c4)c21)C1(c2ccccc2-c2ccccc21)c1ccccc1-3]

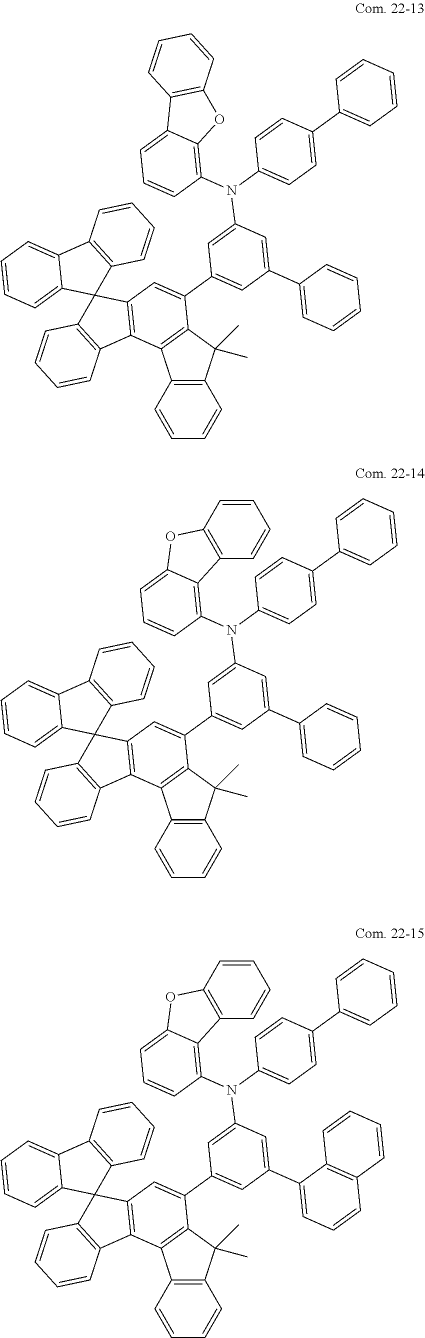 [SMILES: CC1(C)c2ccccc2-c2c3c(cc(-c4cc(-c5ccccc5)cc(N(c5ccc(-c6ccccc6)cc5)c5cccc6c5oc5ccccc56)c4)c21)C1(c2ccccc2-c2ccccc21)c1ccccc1-3 ; CC1(C)c2ccccc2-c2c3c(cc(-c4cc(-c5ccccc5)cc(N(c5ccc(-c6ccccc6)cc5)c5cccc6oc7ccccc7c56)c4)c21)C1(c2ccccc2-c2ccccc21)c1ccccc1-3 ; CC1(C)c2ccccc2-c2c3c(cc(-c4cc(-c5cccc6ccccc56)cc(N(c5ccc(-c6ccccc6)cc5)c5cccc6oc7ccccc7c56)c4)c21)C1(c2ccccc2-c2ccccc21)c1ccccc1-3]

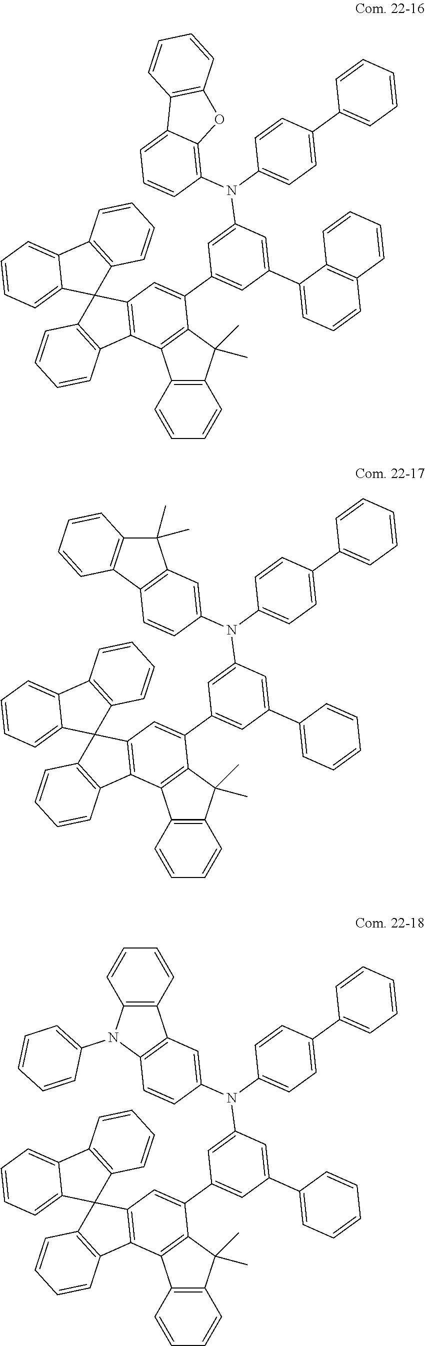 [SMILES: CC1(C)c2ccccc2-c2c3c(cc(-c4cc(-c5cccc6ccccc56)cc(N(c5ccc(-c6ccccc6)cc5)c5cccc6c5oc5ccccc56)c4)c21)C1(c2ccccc2-c2ccccc21)c1ccccc1-3 ; CC1(C)c2ccccc2-c2ccc(N(c3ccc(-c4ccccc4)cc3)c3cc(-c4ccccc4)cc(-c4cc5c(c6c4C(C)(C)c4ccccc4-6)-c4ccccc4C54c5ccccc5-c5ccccc54)c3)cc21 ; CC1(C)c2ccccc2-c2c3c(cc(-c4cc(-c5ccccc5)cc(N(c5ccc(-c6ccccc6)cc5)c5ccc6c(c5)c5ccccc5n6-c5ccccc5)c4)c21)C1(c2ccccc2-c2ccccc21)c1ccccc1-3]

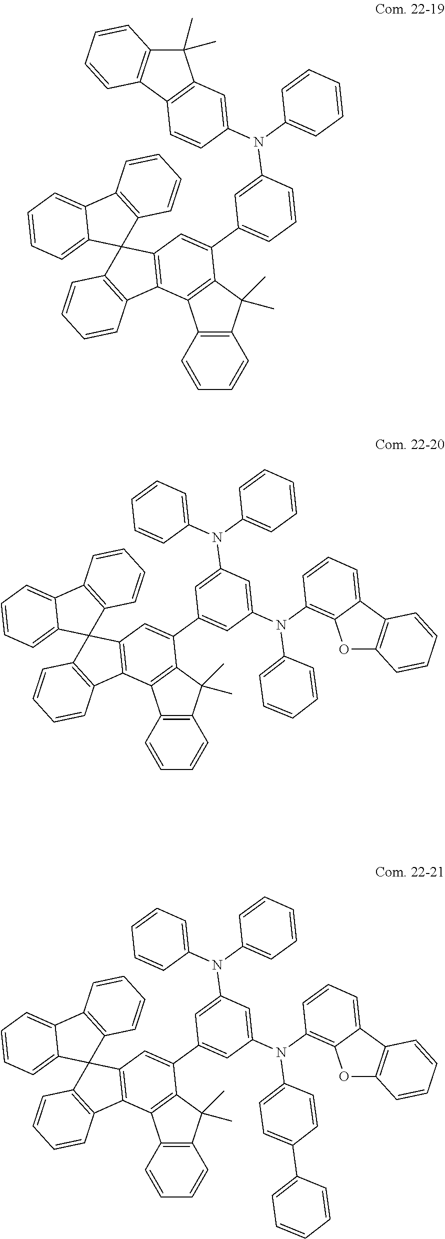 [SMILES: CC1(C)c2ccccc2-c2ccc(N(c3ccccc3)c3cccc(-c4cc5c(c6c4C(C)(C)c4ccccc4-6)-c4ccccc4C54c5ccccc5-c5ccccc54)c3)cc21 ; CC1(C)c2ccccc2-c2c3c(cc(-c4cc(N(c5ccccc5)c5ccccc5)cc(N(c5ccccc5)c5cccc6c5oc5ccccc56)c4)c21)C1(c2ccccc2-c2ccccc21)c1ccccc1-3 ; CC1(C)c2ccccc2-c2c3c(cc(-c4cc(N(c5ccccc5)c5ccccc5)cc(N(c5ccc(-c6ccccc6)cc5)c5cccc6c5oc5ccccc56)c4)c21)C1(c2ccccc2-c2ccccc21)c1ccccc1-3]

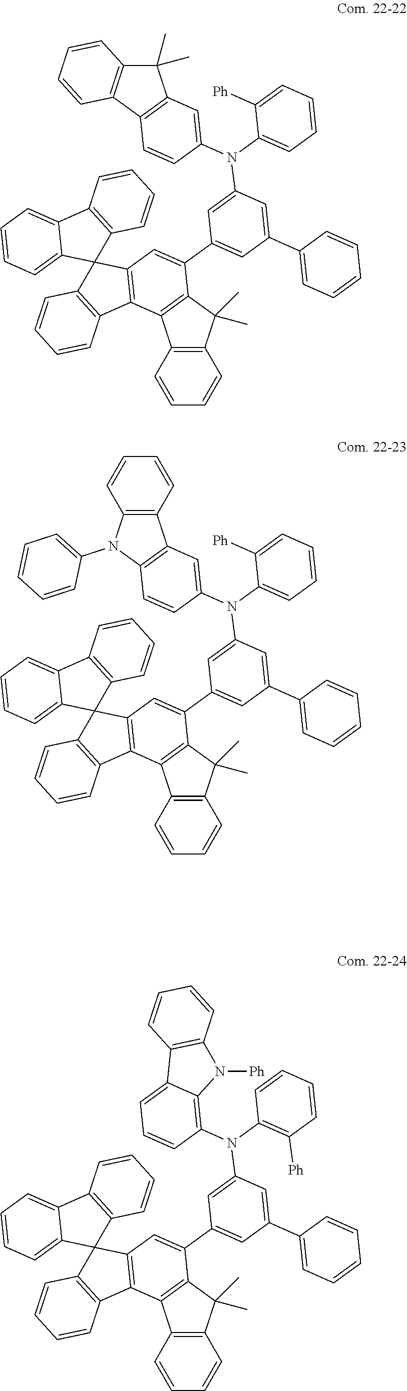 [SMILES: CC1(C)c2ccccc2-c2ccc(N(c3cc(-c4ccccc4)cc(-c4cc5c(c6c4C(C)(C)c4ccccc4-6)-c4ccccc4C54c5ccccc5-c5ccccc54)c3)c3ccccc3-c3ccccc3)cc21 ; CC1(C)c2ccccc2-c2c3c(cc(-c4cc(-c5ccccc5)cc(N(c5ccc6c(c5)c5ccccc5n6-c5ccccc5)c5ccccc5-c5ccccc5)c4)c21)C1(c2ccccc2-c2ccccc21)c1ccccc1-3 ; CC1(C)c2ccccc2-c2c3c(cc(-c4cc(-c5ccccc5)cc(N(c5ccccc5-c5ccccc5)c5cccc6c7ccccc7n(-c7ccccc7)c56)c4)c21)C1(c2ccccc2-c2ccccc21)c1ccccc1-3]

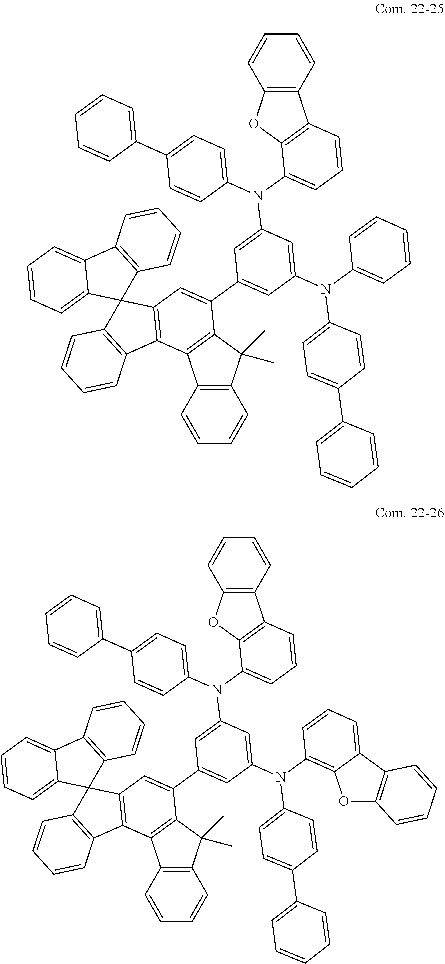 [SMILES: CC1(C)c2ccccc2-c2c3c(cc(-c4cc(N(c5ccccc5)c5ccc(-c6ccccc6)cc5)cc(N(c5ccc(-c6ccccc6)cc5)c5cccc6c5oc5ccccc56)c4)c21)C1(c2ccccc2-c2ccccc21)c1ccccc1-3 ; CC1(C)c2ccccc2-c2c3c(cc(-c4cc(N(c5ccc(-c6ccccc6)cc5)c5cccc6c5oc5ccccc56)cc(N(c5ccc(-c6ccccc6)cc5)c5cccc6c5oc5ccccc56)c4)c21)C1(c2ccccc2-c2ccccc21)c1ccccc1-3]

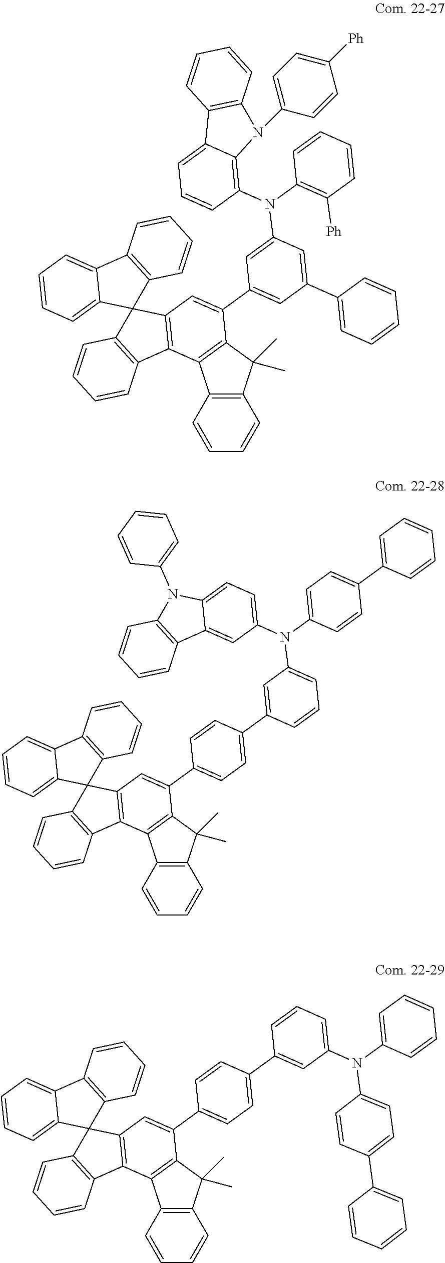 [SMILES: CC1(C)c2ccccc2-c2c3c(cc(-c4cc(-c5ccccc5)cc(N(c5ccccc5-c5ccccc5)c5cccc6c7ccccc7n(-c7ccc(-c8ccccc8)cc7)c56)c4)c21)C1(c2ccccc2-c2ccccc21)c1ccccc1-3 ; CC1(C)c2ccccc2-c2c3c(cc(-c4ccc(-c5cccc(N(c6ccc(-c7ccccc7)cc6)c6ccc7c(c6)c6ccccc6n7-c6ccccc6)c5)cc4)c21)C1(c2ccccc2-c2ccccc21)c1ccccc1-3 ; CC1(C)c2ccccc2-c2c3c(cc(-c4ccc(-c5cccc(N(c6ccccc6)c6ccc(-c7ccccc7)cc6)c5)cc4)c21)C1(c2ccccc2-c2ccccc21)c1ccccc1-3]

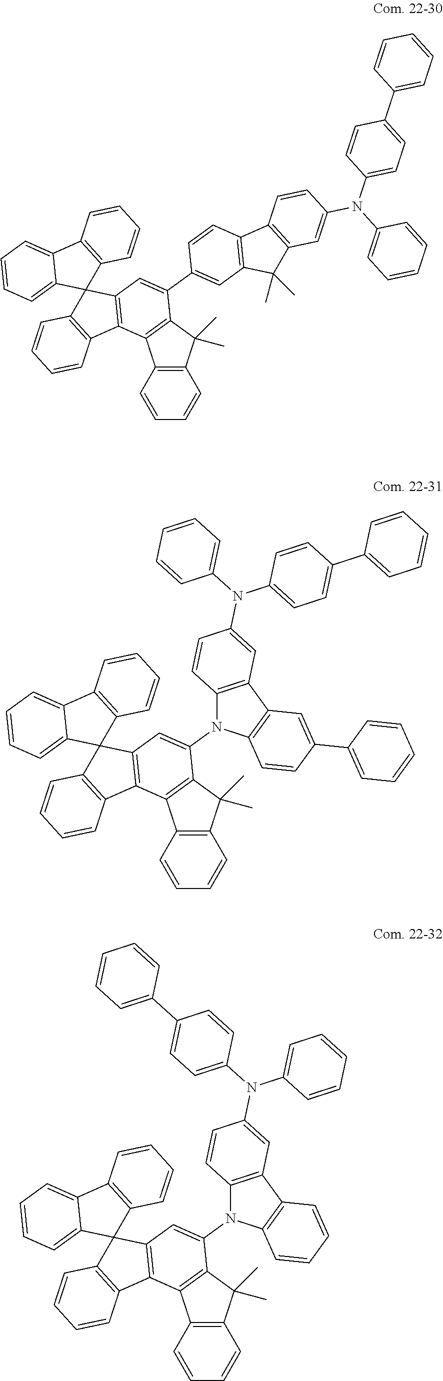 [SMILES: CC1(C)c2cc(-c3cc4c(c5c3C(C)(C)c3ccccc3-5)-c3ccccc3C43c4ccccc4-c4ccccc43)ccc2-c2ccc(N(c3ccccc3)c3ccc(-c4ccccc4)cc3)cc21 ; CC1(C)c2ccccc2-c2c3c(cc(-n4c5ccc(-c6ccccc6)cc5c5cc(N(c6ccccc6)c6ccc(-c7ccccc7)cc6)ccc54)c21)C1(c2ccccc2-c2ccccc21)c1ccccc1-3 ; CC1(C)c2ccccc2-c2c3c(cc(-n4c5ccccc5c5cc(N(c6ccccc6)c6ccc(-c7ccccc7)cc6)ccc54)c21)C1(c2ccccc2-c2ccccc21)c1ccccc1-3]

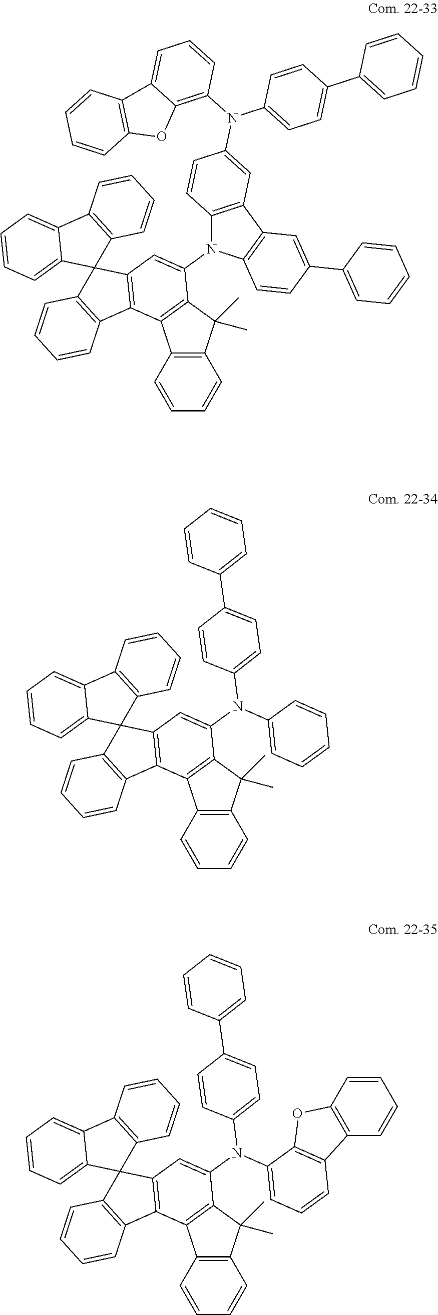 [SMILES: CC1(C)c2ccccc2-c2c3c(cc(-n4c5ccc(-c6ccccc6)cc5c5cc(N(c6ccc(-c7ccccc7)cc6)c6cccc7c6oc6ccccc67)ccc54)c21)C1(c2ccccc2-c2ccccc21)c1ccccc1-3 ; CC1(C)c2ccccc2-c2c3c(cc(N(c4ccccc4)c4ccc(-c5ccccc5)cc4)c21)C1(c2ccccc2-c2ccccc21)c1ccccc1-3 ; CC1(C)c2ccccc2-c2c3c(cc(N(c4ccc(-c5ccccc5)cc4)c4cccc5c4oc4ccccc45)c21)C1(c2ccccc2-c2ccccc21)c1ccccc1-3]

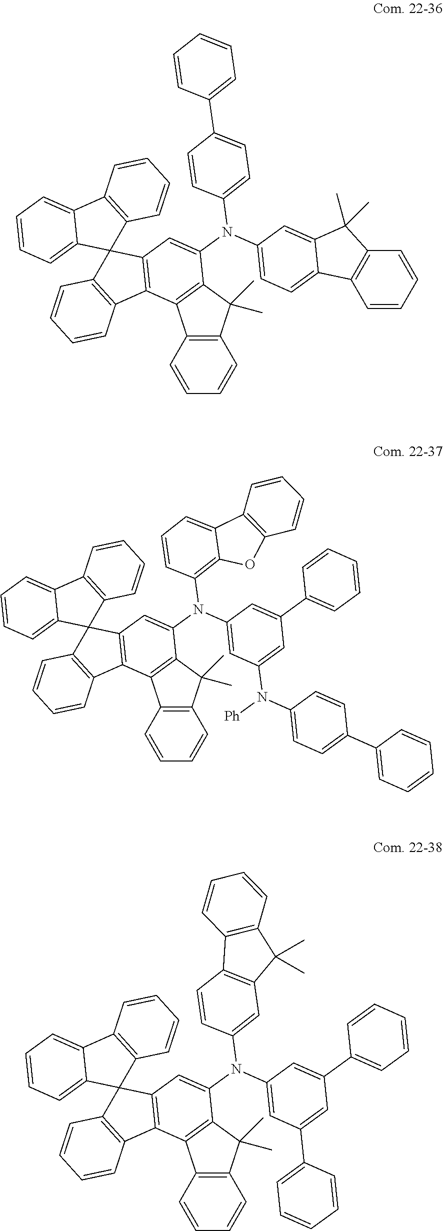 [SMILES: CC1(C)c2ccccc2-c2ccc(N(c3ccc(-c4ccccc4)cc3)c3cc4c(c5c3C(C)(C)c3ccccc3-5)-c3ccccc3C43c4ccccc4-c4ccccc43)cc21 ; CC1(C)c2ccccc2-c2c3c(cc(N(c4cc(-c5ccccc5)cc(N(c5ccccc5)c5ccc(-c6ccccc6)cc5)c4)c4cccc5c4oc4ccccc45)c21)C1(c2ccccc2-c2ccccc21)c1ccccc1-3 ; CC1(C)c2ccccc2-c2ccc(N(c3cc(-c4ccccc4)cc(-c4ccccc4)c3)c3cc4c(c5c3C(C)(C)c3ccccc3-5)-c3ccccc3C43c4ccccc4-c4ccccc43)cc21]

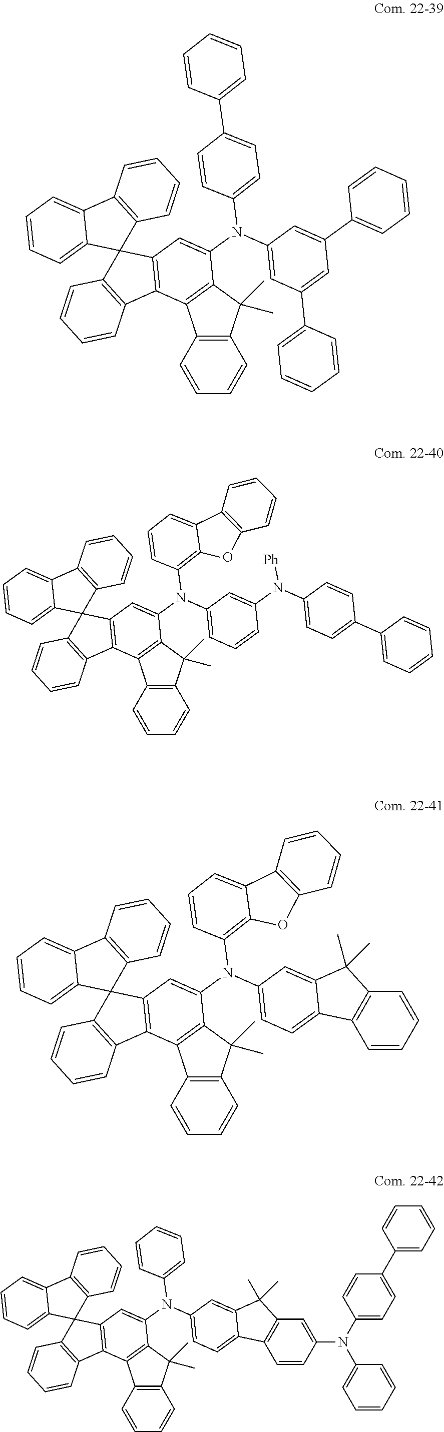 [SMILES: CC1(C)c2ccccc2-c2c3c(cc(N(c4ccc(-c5ccccc5)cc4)c4cc(-c5ccccc5)cc(-c5ccccc5)c4)c21)C1(c2ccccc2-c2ccccc21)c1ccccc1-3 ; CC1(C)c2ccccc2-c2c3c(cc(N(c4cccc(N(c5ccccc5)c5ccc(-c6ccccc6)cc5)c4)c4cccc5c4oc4ccccc45)c21)C1(c2ccccc2-c2ccccc21)c1ccccc1-3 ; CC1(C)c2ccccc2-c2ccc(N(c3cc4c(c5c3C(C)(C)c3ccccc3-5)-c3ccccc3C43c4ccccc4-c4ccccc43)c3cccc4c3oc3ccccc34)cc21 ; CC1(C)c2cc(N(c3ccccc3)c3ccc(-c4ccccc4)cc3)ccc2-c2ccc(N(c3ccccc3)c3cc4c(c5c3C(C)(C)c3ccccc3-5)-c3ccccc3C43c4ccccc4-c4ccccc43)cc21]

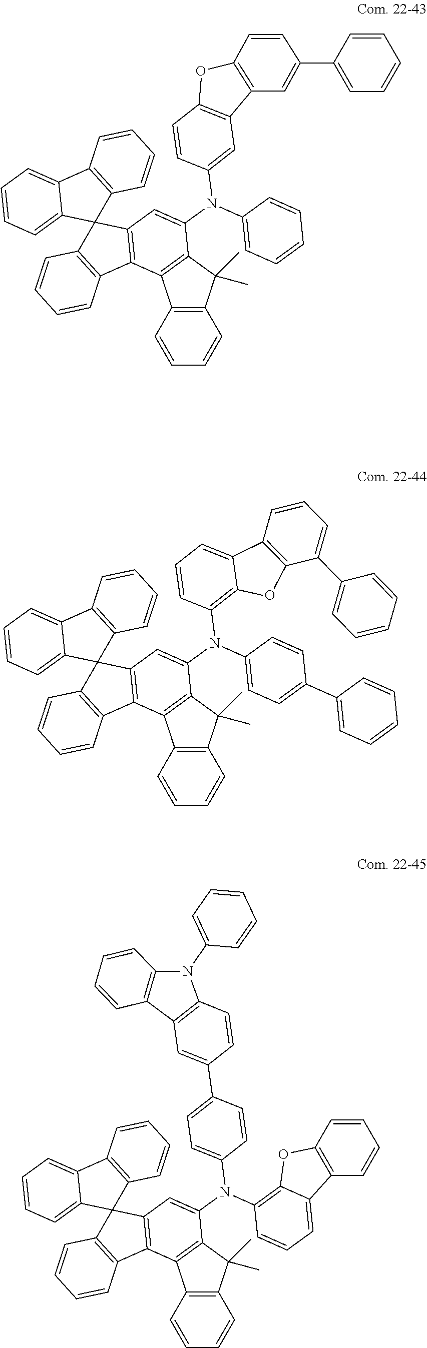 [SMILES: CC1(C)c2ccccc2-c2c3c(cc(N(c4ccccc4)c4ccc5oc6ccc(-c7ccccc7)cc6c5c4)c21)C1(c2ccccc2-c2ccccc21)c1ccccc1-3 ; CC1(C)c2ccccc2-c2c3c(cc(N(c4ccc(-c5ccccc5)cc4)c4cccc5c4oc4c(-c6ccccc6)cccc45)c21)C1(c2ccccc2-c2ccccc21)c1ccccc1-3 ; CC1(C)c2ccccc2-c2c3c(cc(N(c4ccc(-c5ccc6c(c5)c5ccccc5n6-c5ccccc5)cc4)c4cccc5c4oc4ccccc45)c21)C1(c2ccccc2-c2ccccc21)c1ccccc1-3]

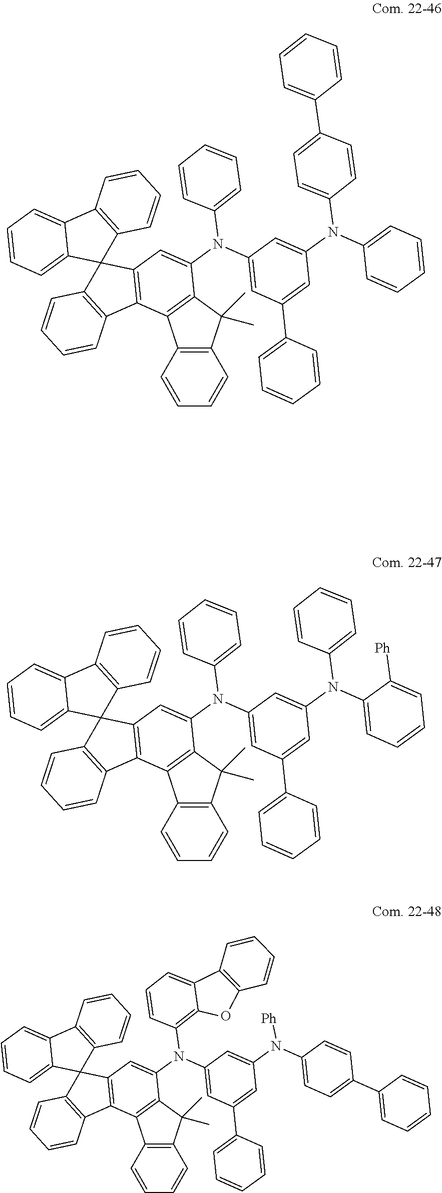 [SMILES: CC1(C)c2ccccc2-c2c3c(cc(N(c4ccccc4)c4cc(-c5ccccc5)cc(N(c5ccccc5)c5ccc(-c6ccccc6)cc5)c4)c21)C1(c2ccccc2-c2ccccc21)c1ccccc1-3 ; CC1(C)c2ccccc2-c2c3c(cc(N(c4ccccc4)c4cc(-c5ccccc5)cc(N(c5ccccc5)c5ccccc5-c5ccccc5)c4)c21)C1(c2ccccc2-c2ccccc21)c1ccccc1-3 ; CC1(C)c2ccccc2-c2c3c(cc(N(c4cc(-c5ccccc5)cc(N(c5ccccc5)c5ccc(-c6ccccc6)cc5)c4)c4cccc5c4oc4ccccc45)c21)C1(c2ccccc2-c2ccccc21)c1ccccc1-3]

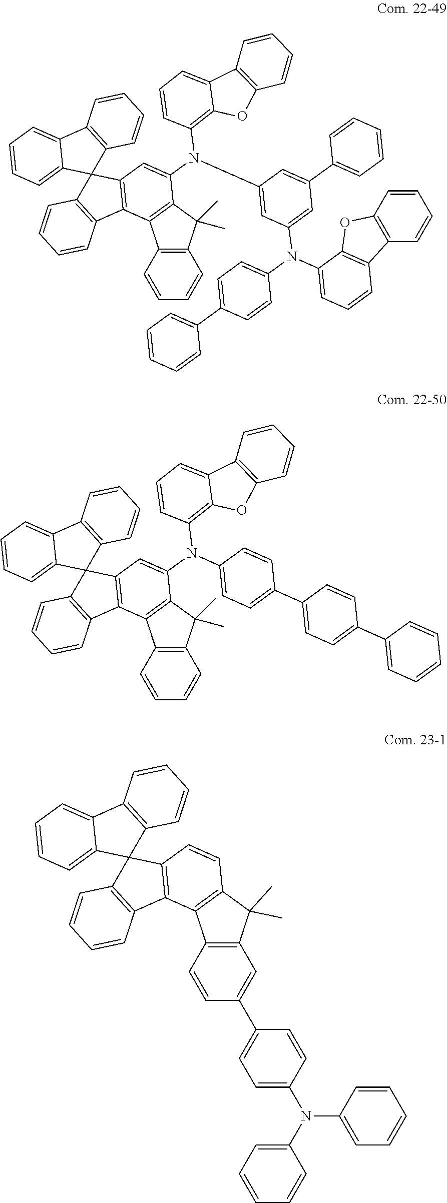 [SMILES: CC1(C)c2ccccc2-c2c3c(cc(N(c4cc(-c5ccccc5)cc(N(c5ccc(-c6ccccc6)cc5)c5cccc6c5oc5ccccc56)c4)c4cccc5c4oc4ccccc45)c21)C1(c2ccccc2-c2ccccc21)c1ccccc1-3 ; CC1(C)c2ccccc2-c2c3c(cc(N(c4ccc(-c5ccc(-c6ccccc6)cc5)cc4)c4cccc5c4oc4ccccc45)c21)C1(c2ccccc2-c2ccccc21)c1ccccc1-3 ; CC1(C)c2cc(-c3ccc(N(c4ccccc4)c4ccccc4)cc3)ccc2-c2c1ccc1c2-c2ccccc2C12c1ccccc1-c1ccccc12]

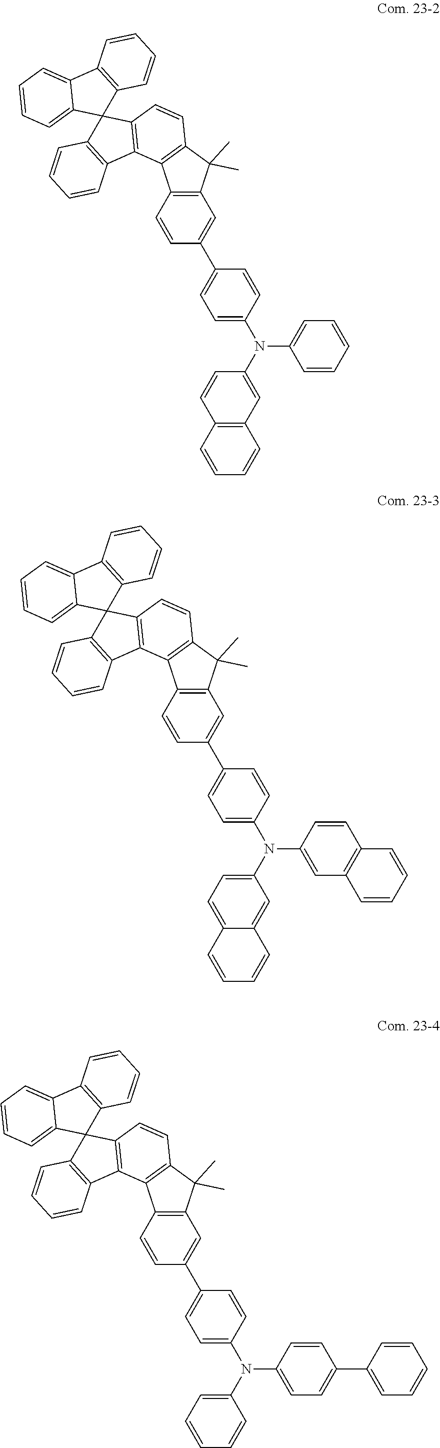 [SMILES: CC1(C)c2cc(-c3ccc(N(c4ccccc4)c4ccc5ccccc5c4)cc3)ccc2-c2c1ccc1c2-c2ccccc2C12c1ccccc1-c1ccccc12 ; CC1(C)c2cc(-c3ccc(N(c4ccc5ccccc5c4)c4ccc5ccccc5c4)cc3)ccc2-c2c1ccc1c2-c2ccccc2C12c1ccccc1-c1ccccc12 ; CC1(C)c2cc(-c3ccc(N(c4ccccc4)c4ccc(-c5ccccc5)cc4)cc3)ccc2-c2c1ccc1c2-c2ccccc2C12c1ccccc1-c1ccccc12]

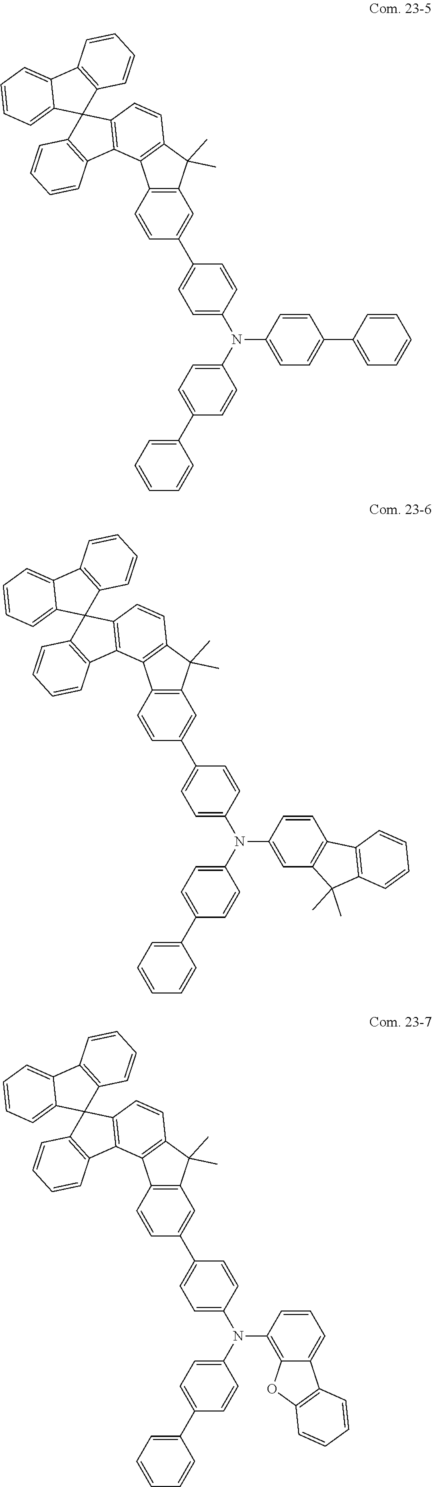 [SMILES: CC1(C)c2cc(-c3ccc(N(c4ccc(-c5ccccc5)cc4)c4ccc(-c5ccccc5)cc4)cc3)ccc2-c2c1ccc1c2-c2ccccc2C12c1ccccc1-c1ccccc12 ; CC1(C)c2ccccc2-c2ccc(N(c3ccc(-c4ccccc4)cc3)c3ccc(-c4ccc5c(c4)C(C)(C)c4ccc6c(c4-5)-c4ccccc4C64c5ccccc5-c5ccccc54)cc3)cc21 ; CC1(C)c2cc(-c3ccc(N(c4ccc(-c5ccccc5)cc4)c4cccc5c4oc4ccccc45)cc3)ccc2-c2c1ccc1c2-c2ccccc2C12c1ccccc1-c1ccccc12]

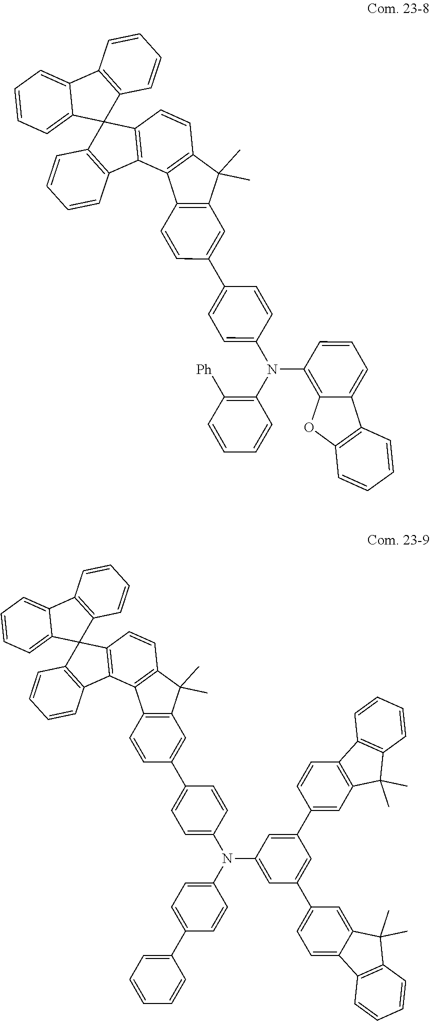 [SMILES: CC1(C)c2cc(-c3ccc(N(c4ccccc4-c4ccccc4)c4cccc5c4oc4ccccc45)cc3)ccc2-c2c1ccc1c2-c2ccccc2C12c1ccccc1-c1ccccc12 ; CC1(C)c2ccccc2-c2ccc(-c3cc(-c4ccc5c(c4)C(C)(C)c4ccccc4-5)cc(N(c4ccc(-c5ccccc5)cc4)c4ccc(-c5ccc6c(c5)C(C)(C)c5ccc7c(c5-6)-c5ccccc5C75c6ccccc6-c6ccccc65)cc4)c3)cc21]

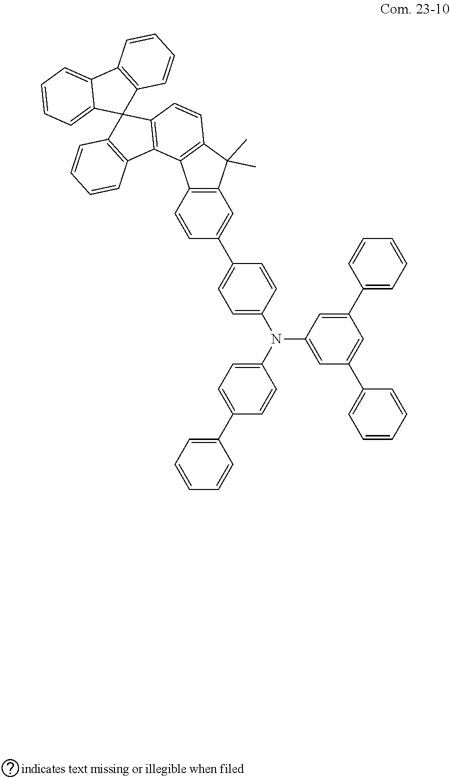 [SMILES: CC1(C)c2cc(-c3ccc(N(c4ccc(-c5ccccc5)cc4)c4cc(-c5ccccc5)cc(-c5ccccc5)c4)cc3)ccc2-c2c1ccc1c2-c2ccccc2C12c1ccccc1-c1ccccc12]

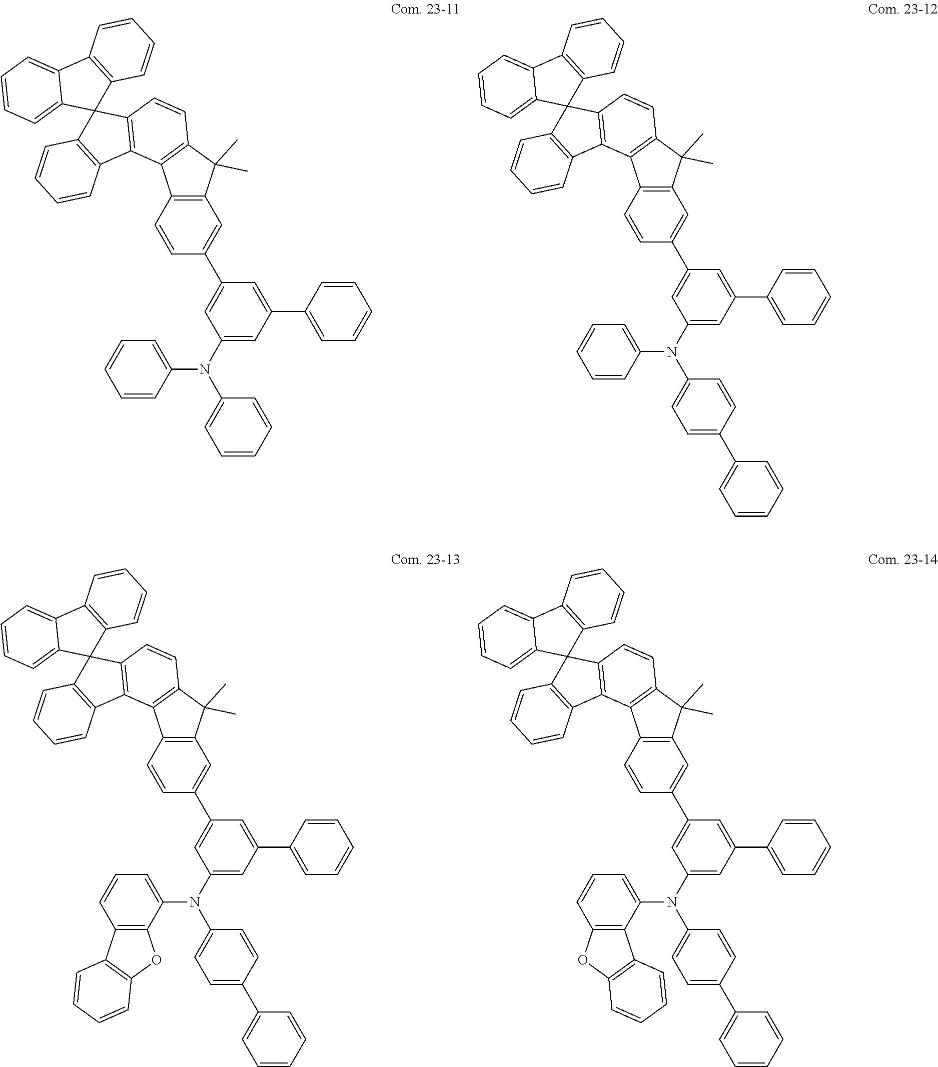 [SMILES: CC1(C)c2cc(-c3cc(-c4ccccc4)cc(N(c4ccccc4)c4ccccc4)c3)ccc2-c2c1ccc1c2-c2ccccc2C12c1ccccc1-c1ccccc12 ; CC1(C)c2cc(-c3cc(-c4ccccc4)cc(N(c4ccccc4)c4ccc(-c5ccccc5)cc4)c3)ccc2-c2c1ccc1c2-c2ccccc2C12c1ccccc1-c1ccccc12 ; CC1(C)c2cc(-c3cc(-c4ccccc4)cc(N(c4ccc(-c5ccccc5)cc4)c4cccc5c4oc4ccccc45)c3)ccc2-c2c1ccc1c2-c2ccccc2C12c1ccccc1-c1ccccc12 ; CC1(C)c2cc(-c3cc(-c4ccccc4)cc(N(c4ccc(-c5ccccc5)cc4)c4cccc5oc6ccccc6c45)c3)ccc2-c2c1ccc1c2-c2ccccc2C12c1ccccc1-c1ccccc12]

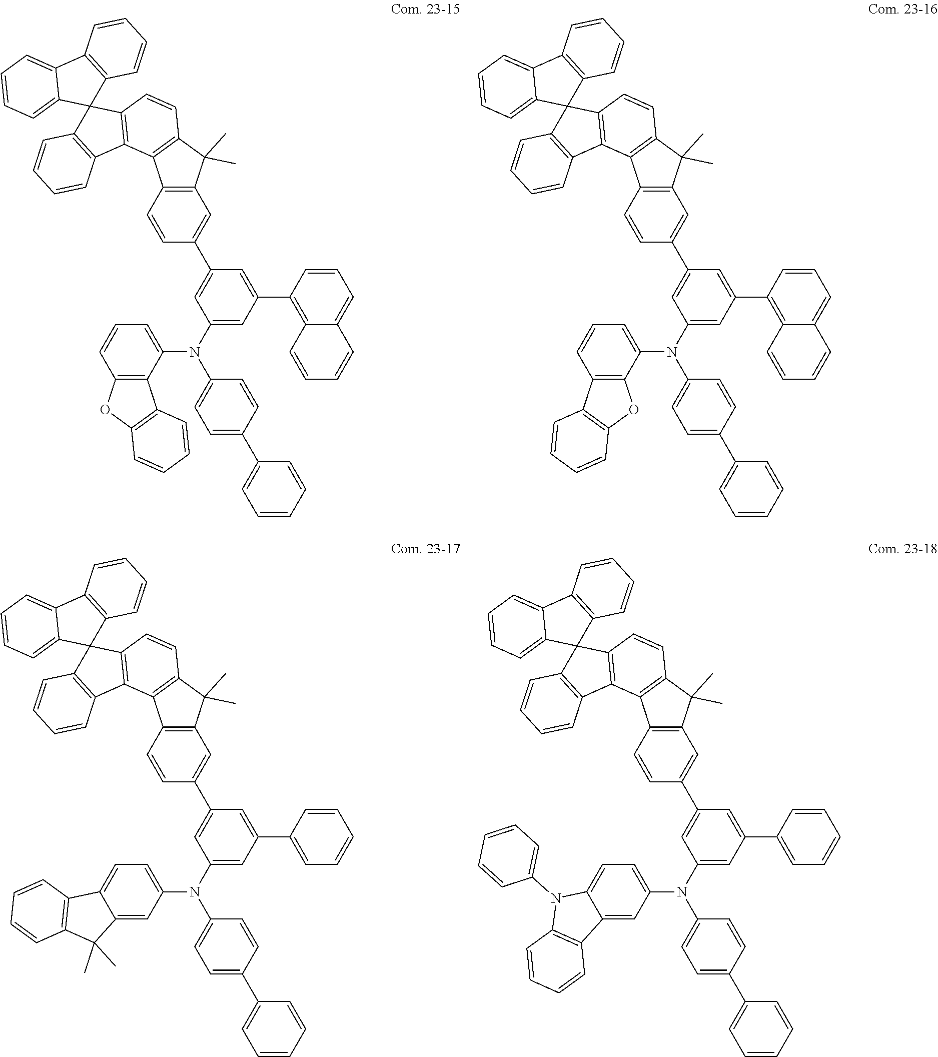

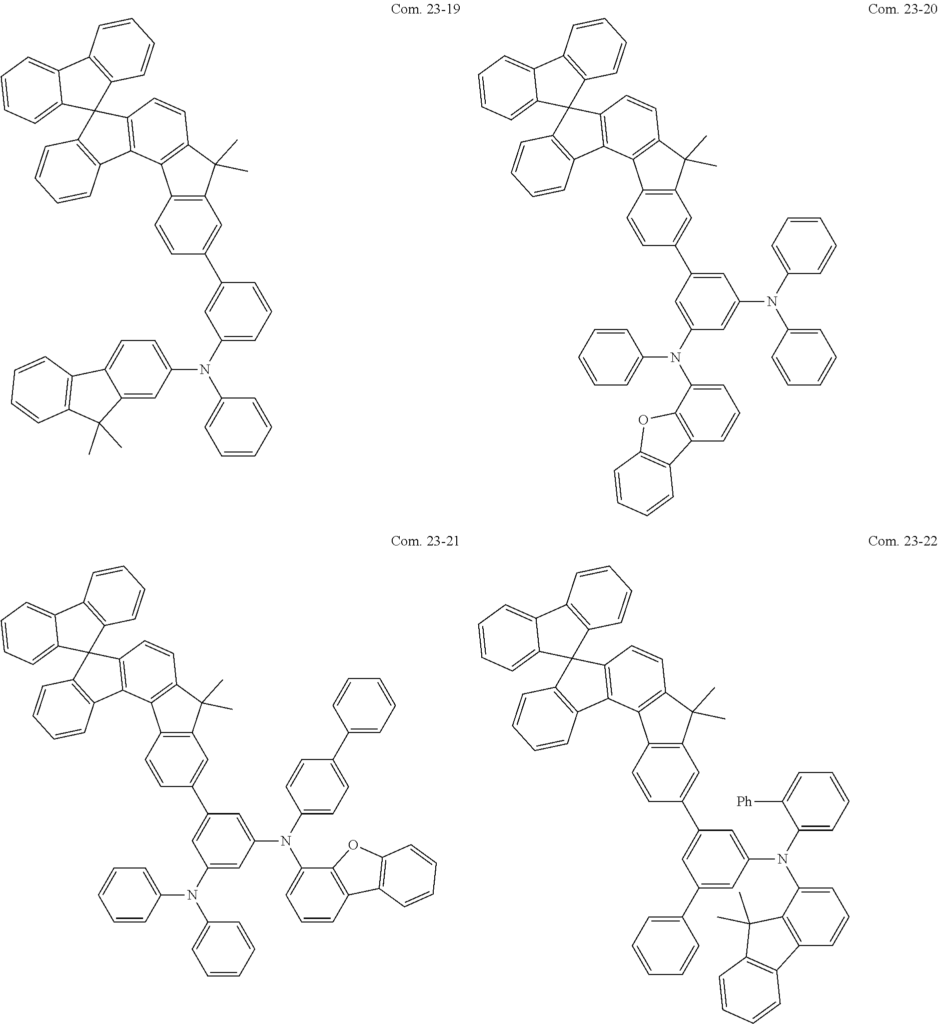 [SMILES: CC1(C)c2ccccc2-c2ccc(N(c3ccccc3)c3cccc(-c4ccc5c(c4)C(C)(C)c4ccc6c(c4-5)-c4ccccc4C64c5ccccc5-c5ccccc54)c3)cc21 ; CC1(C)c2cc(-c3cc(N(c4ccccc4)c4ccccc4)cc(N(c4ccccc4)c4cccc5c4oc4ccccc45)c3)ccc2-c2c1ccc1c2-c2ccccc2C12c1ccccc1-c1ccccc12 ; CC1(C)c2cc(-c3cc(N(c4ccccc4)c4ccccc4)cc(N(c4ccc(-c5ccccc5)cc4)c4cccc5c4oc4ccccc45)c3)ccc2-c2c1ccc1c2-c2ccccc2C12c1ccccc1-c1ccccc12 ; CC1(C)c2cc(-c3cc(-c4ccccc4)cc(N(c4ccccc4-c4ccccc4)c4cccc5c4C(C)(C)c4ccccc4-5)c3)ccc2-c2c1ccc1c2-c2ccccc2C12c1ccccc1-c1ccccc12]

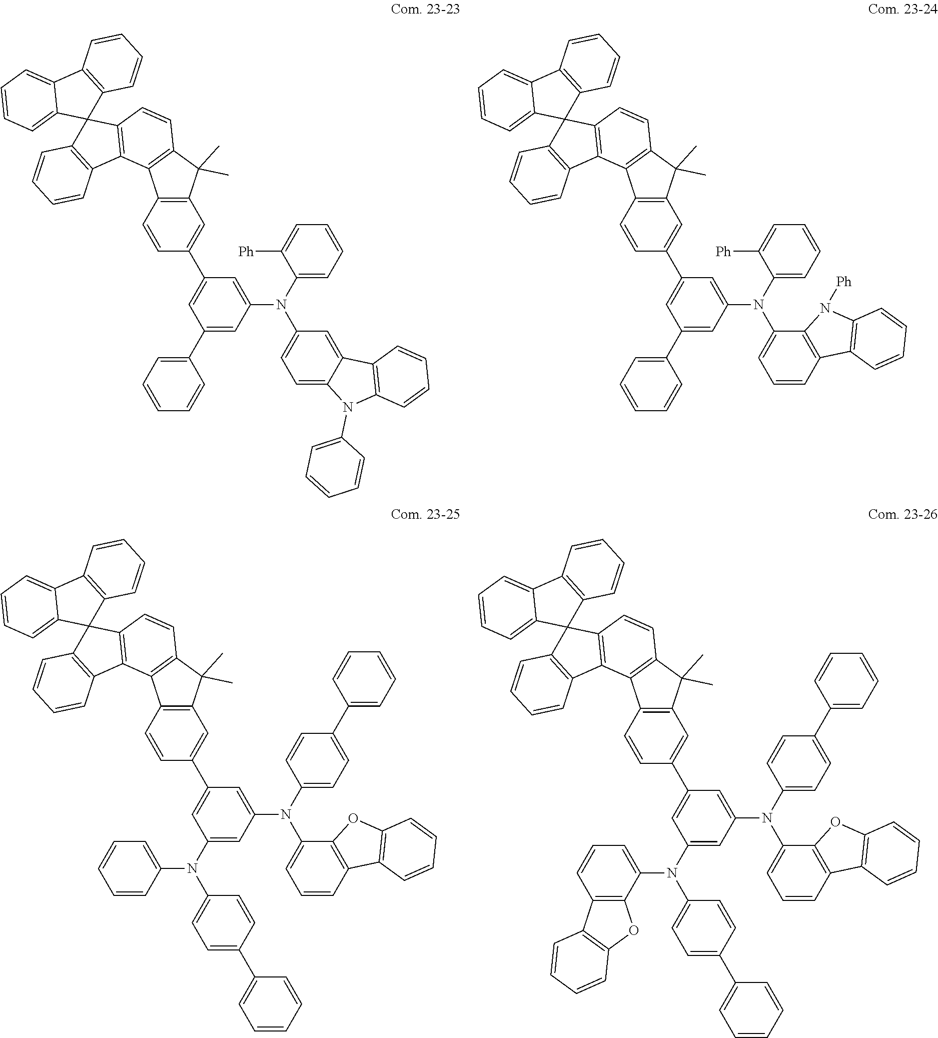

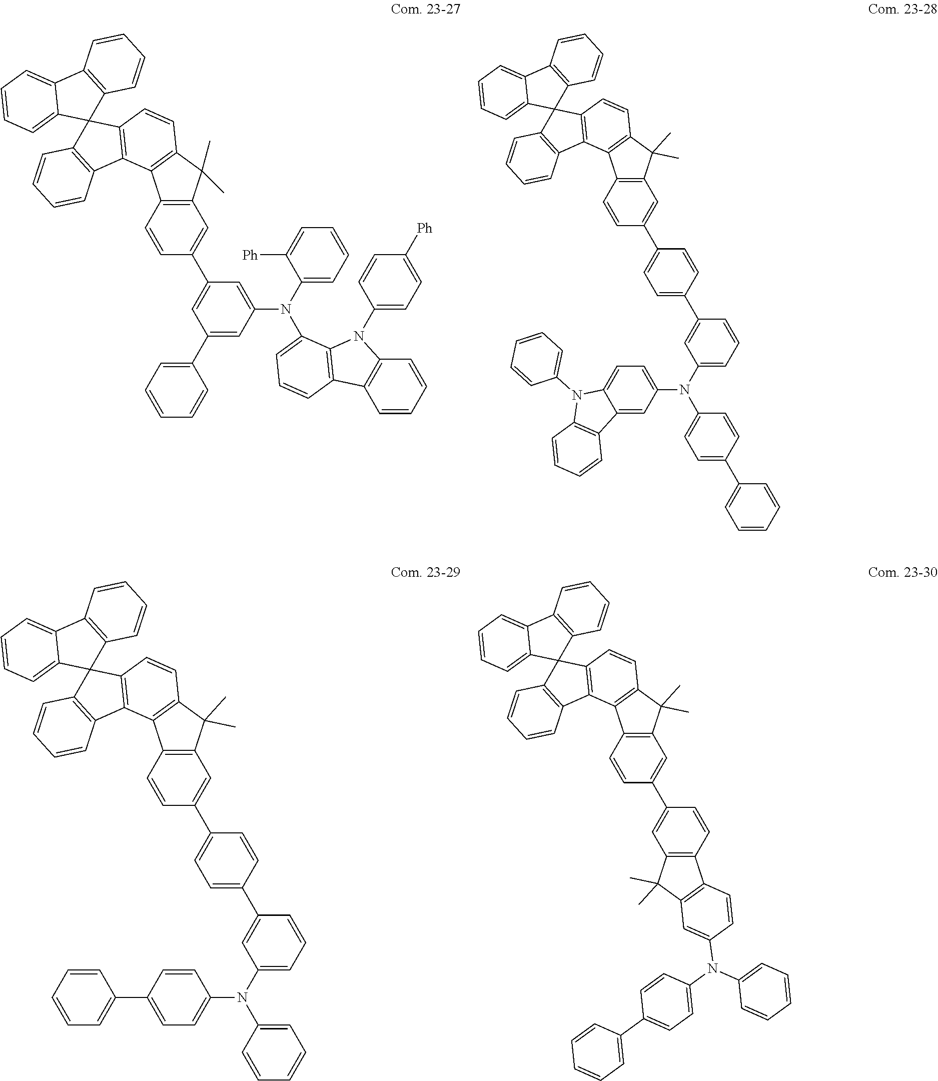

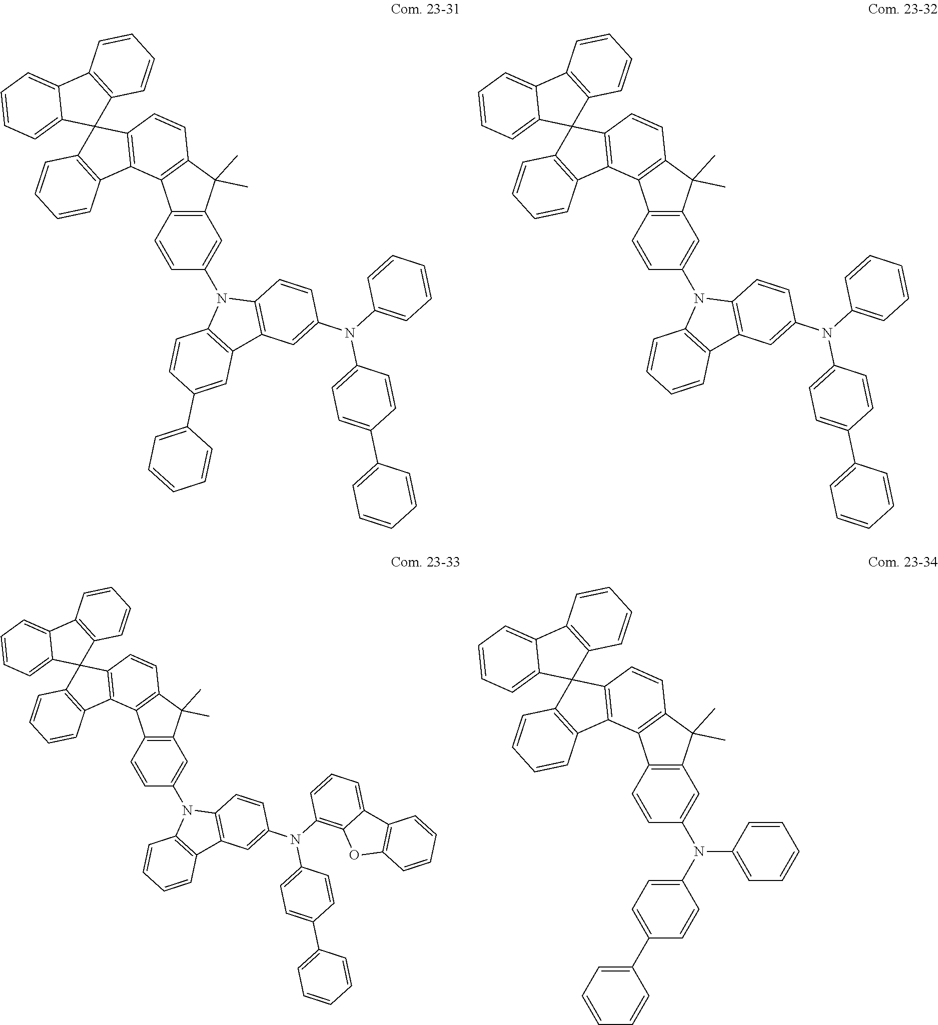 [SMILES: CC1(C)c2cc(-n3c4ccc(-c5ccccc5)cc4c4cc(N(c5ccccc5)c5ccc(-c6ccccc6)cc5)ccc43)ccc2-c2c1ccc1c2-c2ccccc2C12c1ccccc1-c1ccccc12 ; CC1(C)c2cc(-n3c4ccccc4c4cc(N(c5ccccc5)c5ccc(-c6ccccc6)cc5)ccc43)ccc2-c2c1ccc1c2-c2ccccc2C12c1ccccc1-c1ccccc12 ; CC1(C)c2cc(-n3c4ccccc4c4cc(N(c5ccc(-c6ccccc6)cc5)c5cccc6c5oc5ccccc56)ccc43)ccc2-c2c1ccc1c2-c2ccccc2C12c1ccccc1-c1ccccc12 ; CC1(C)c2cc(N(c3ccccc3)c3ccc(-c4ccccc4)cc3)ccc2-c2c1ccc1c2-c2ccccc2C12c1ccccc1-c1ccccc12]

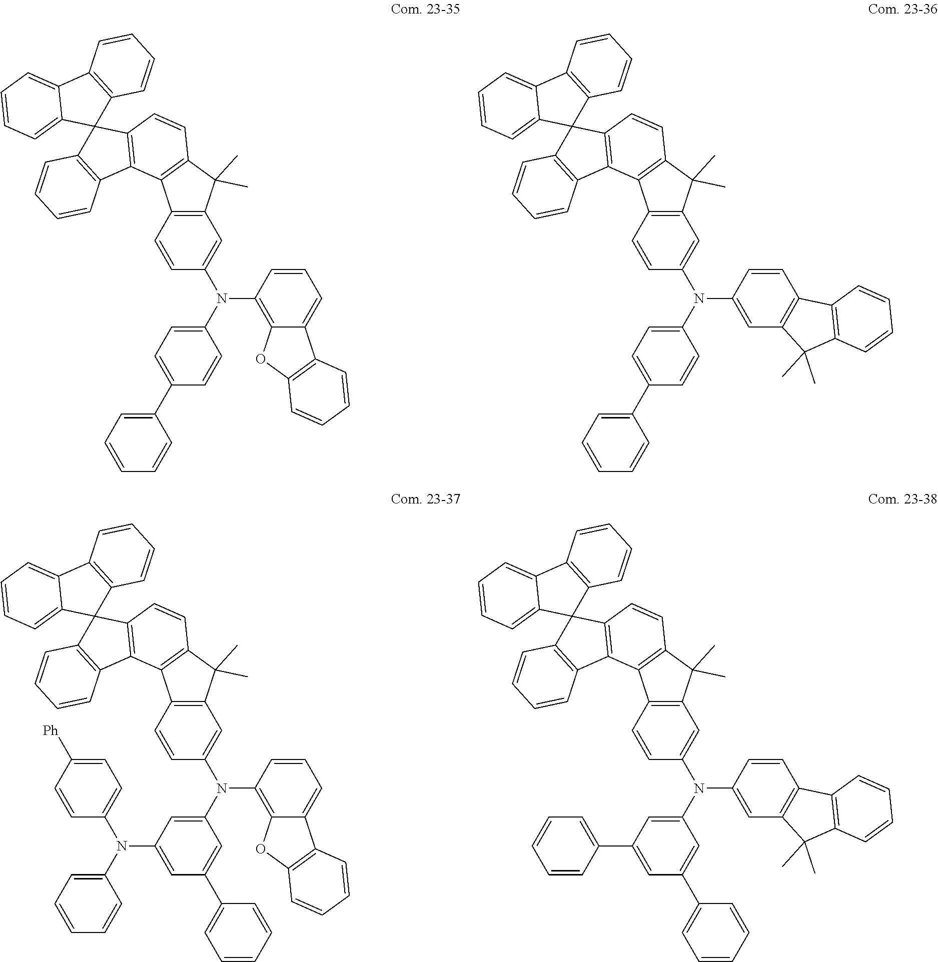 [SMILES: CC1(C)c2cc(N(c3ccc(-c4ccccc4)cc3)c3cccc4c3oc3ccccc34)ccc2-c2c1ccc1c2-c2ccccc2C12c1ccccc1-c1ccccc12 ; CC1(C)c2ccccc2-c2ccc(N(c3ccc(-c4ccccc4)cc3)c3ccc4c(c3)C(C)(C)c3ccc5c(c3-4)-c3ccccc3C53c4ccccc4-c4ccccc43)cc21 ; CC1(C)c2cc(N(c3cc(-c4ccccc4)cc(N(c4ccccc4)c4ccc(-c5ccccc5)cc4)c3)c3cccc4c3oc3ccccc34)ccc2-c2c1ccc1c2-c2ccccc2C12c1ccccc1-c1ccccc12 ; CC1(C)c2ccccc2-c2ccc(N(c3cc(-c4ccccc4)cc(-c4ccccc4)c3)c3ccc4c(c3)C(C)(C)c3ccc5c(c3-4)-c3ccccc3C53c4ccccc4-c4ccccc43)cc21]

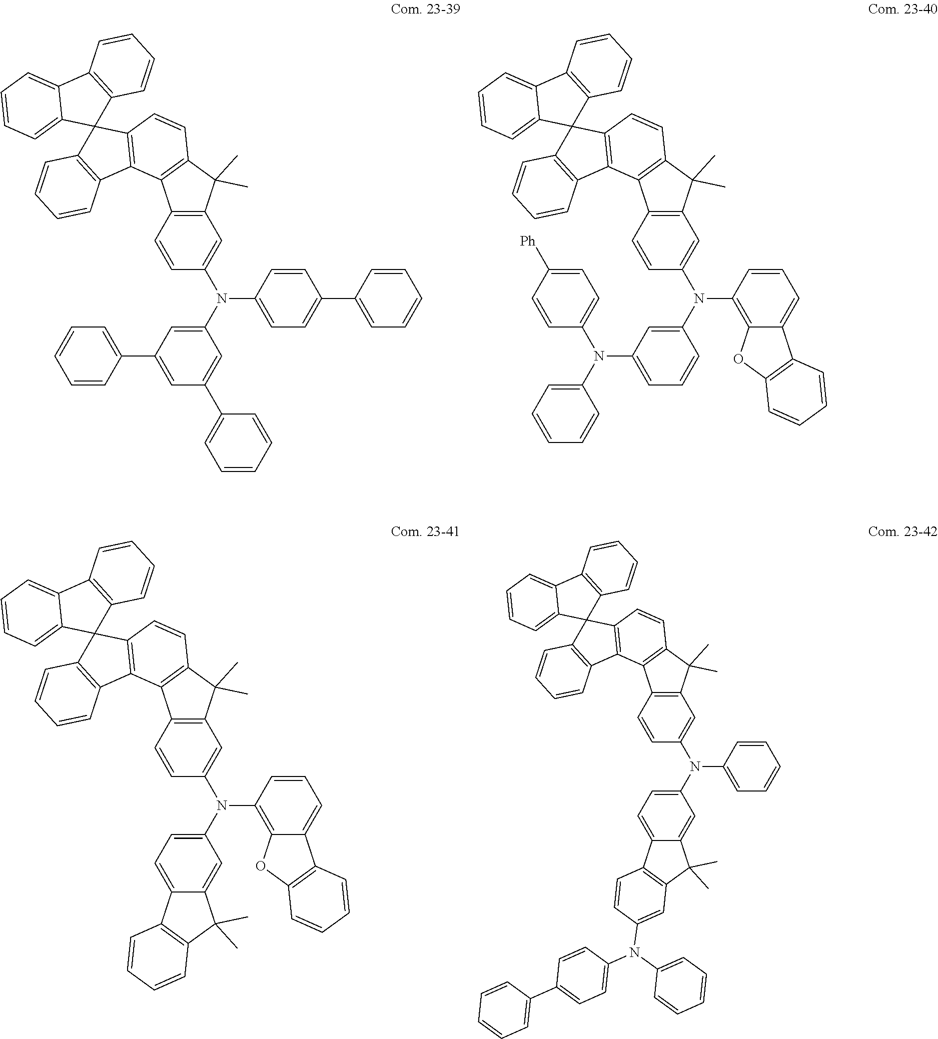 [SMILES: CC1(C)c2cc(N(c3ccc(-c4ccccc4)cc3)c3cc(-c4ccccc4)cc(-c4ccccc4)c3)ccc2-c2c1ccc1c2-c2ccccc2C12c1ccccc1-c1ccccc12 ; CC1(C)c2cc(N(c3cccc(N(c4ccccc4)c4ccc(-c5ccccc5)cc4)c3)c3cccc4c3oc3ccccc34)ccc2-c2c1ccc1c2-c2ccccc2C12c1ccccc1-c1ccccc12 ; CC1(C)c2ccccc2-c2ccc(N(c3ccc4c(c3)C(C)(C)c3ccc5c(c3-4)-c3ccccc3C53c4ccccc4-c4ccccc43)c3cccc4c3oc3ccccc34)cc21 ; CC1(C)c2cc(N(c3ccccc3)c3ccc(-c4ccccc4)cc3)ccc2-c2ccc(N(c3ccccc3)c3ccc4c(c3)C(C)(C)c3ccc5c(c3-4)-c3ccccc3C53c4ccccc4-c4ccccc43)cc21]

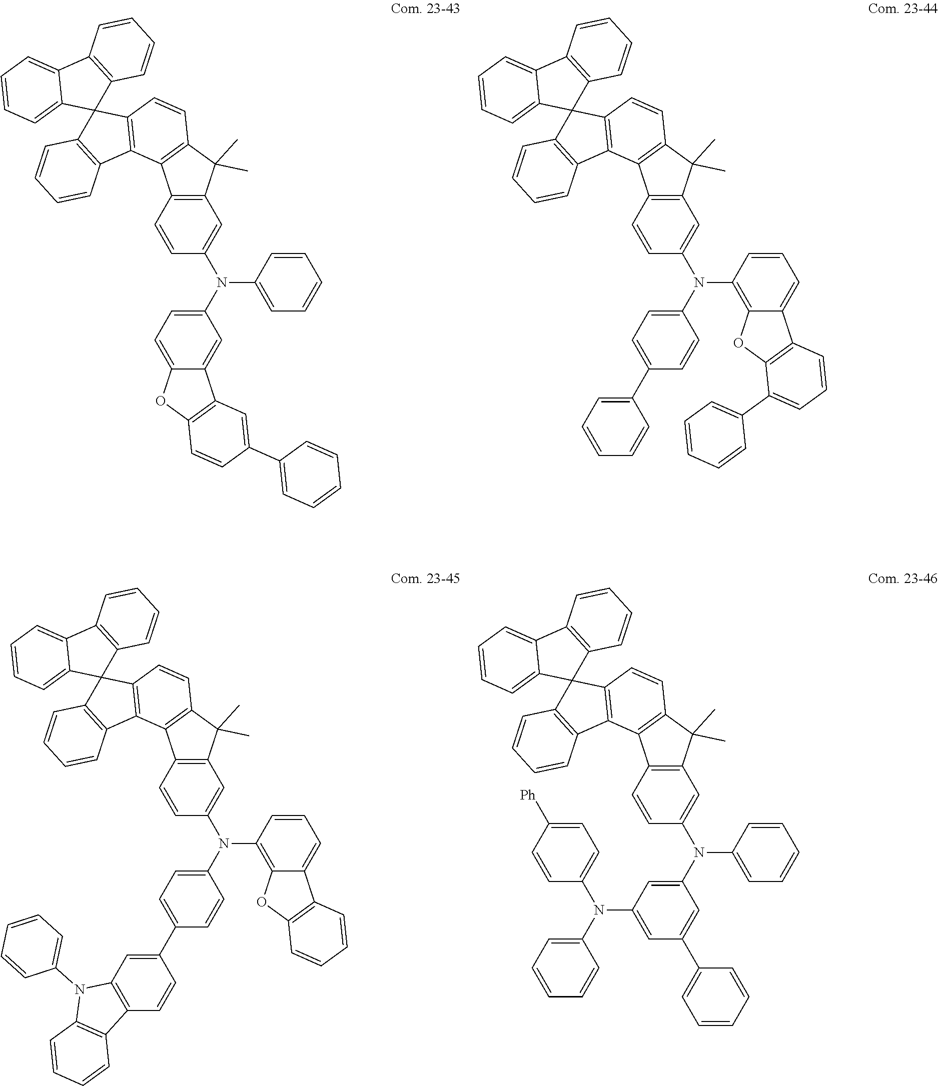 [SMILES: CC1(C)c2cc(N(c3ccccc3)c3ccc4oc5ccc(-c6ccccc6)cc5c4c3)ccc2-c2c1ccc1c2-c2ccccc2C12c1ccccc1-c1ccccc12 ; CC1(C)c2cc(N(c3ccc(-c4ccccc4)cc3)c3cccc4c3oc3c(-c5ccccc5)cccc34)ccc2-c2c1ccc1c2-c2ccccc2C12c1ccccc1-c1ccccc12 ; CC1(C)c2cc(N(c3ccc(-c4ccc5c6ccccc6n(-c6ccccc6)c5c4)cc3)c3cccc4c3oc3ccccc34)ccc2-c2c1ccc1c2-c2ccccc2C12c1ccccc1-c1ccccc12 ; CC1(C)c2cc(N(c3ccccc3)c3cc(-c4ccccc4)cc(N(c4ccccc4)c4ccc(-c5ccccc5)cc4)c3)ccc2-c2c1ccc1c2-c2ccccc2C12c1ccccc1-c1ccccc12]

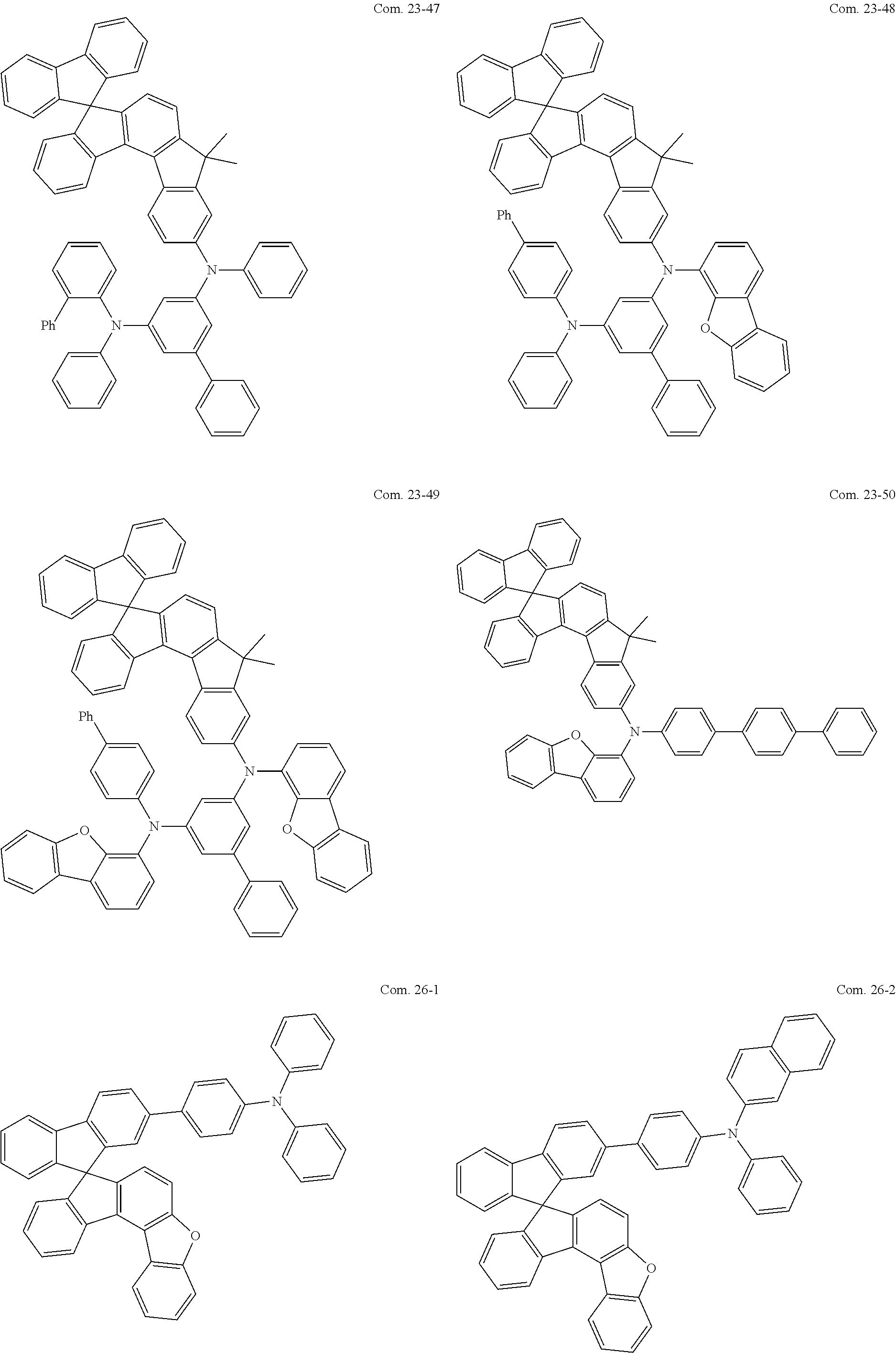

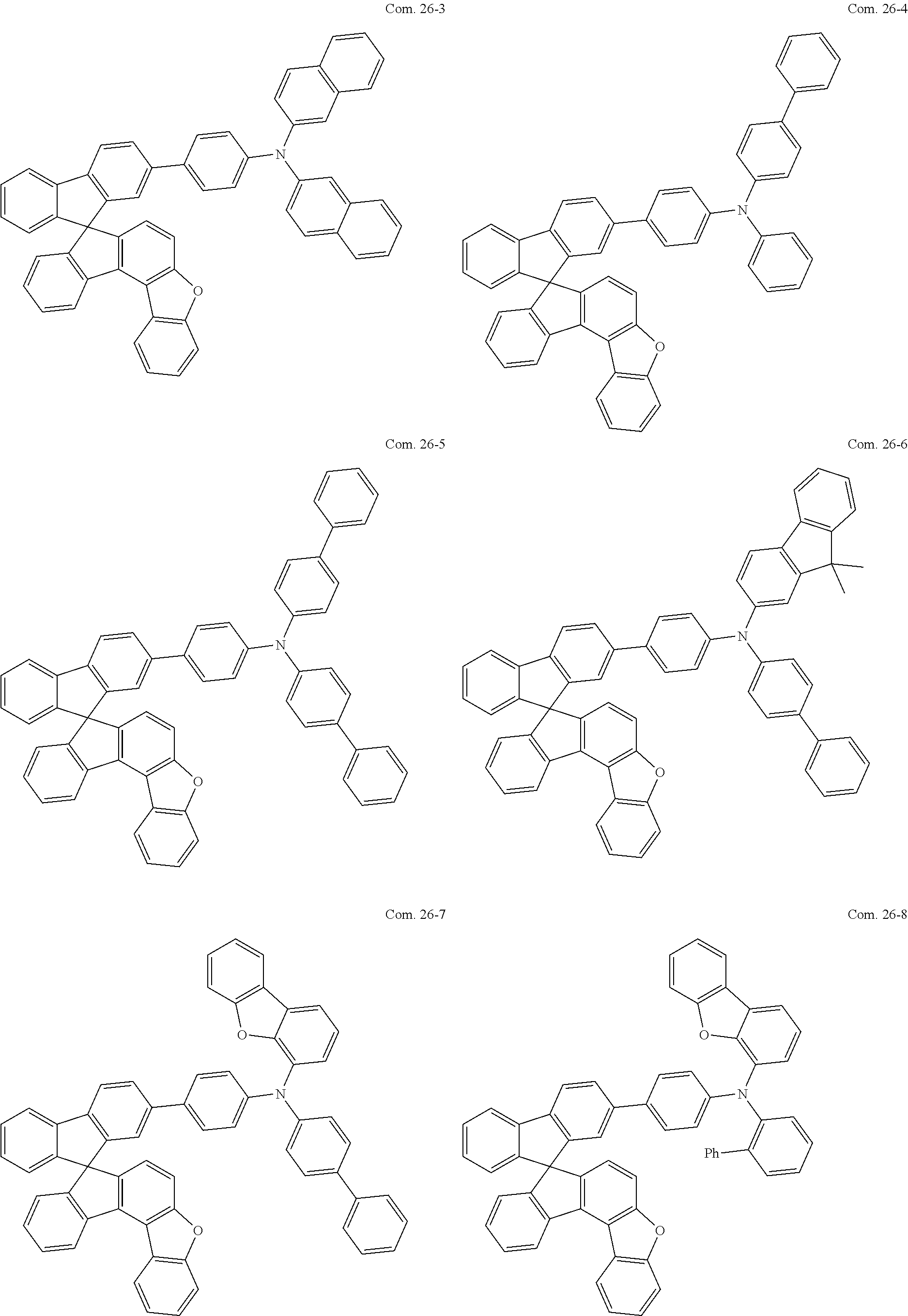

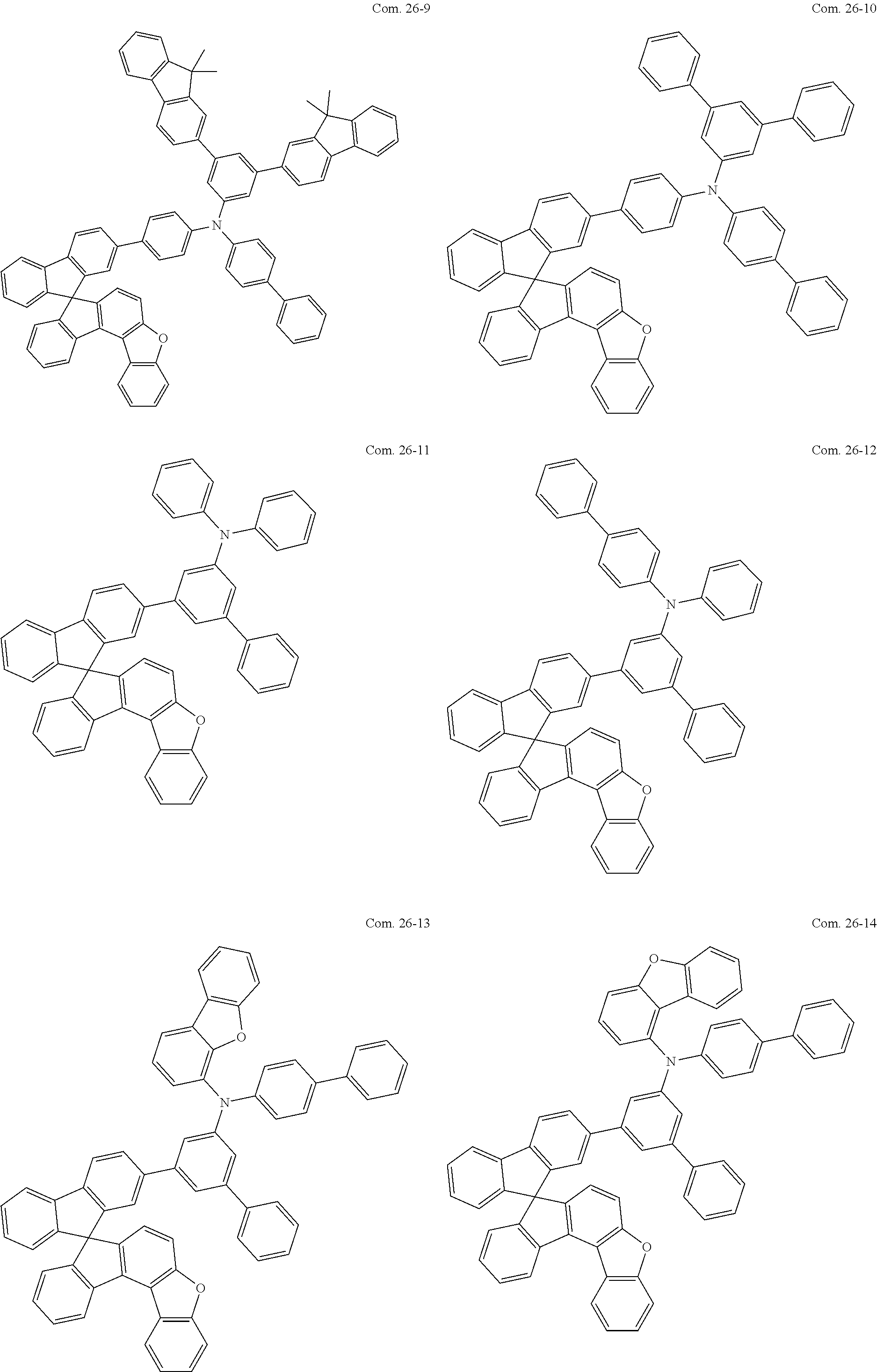

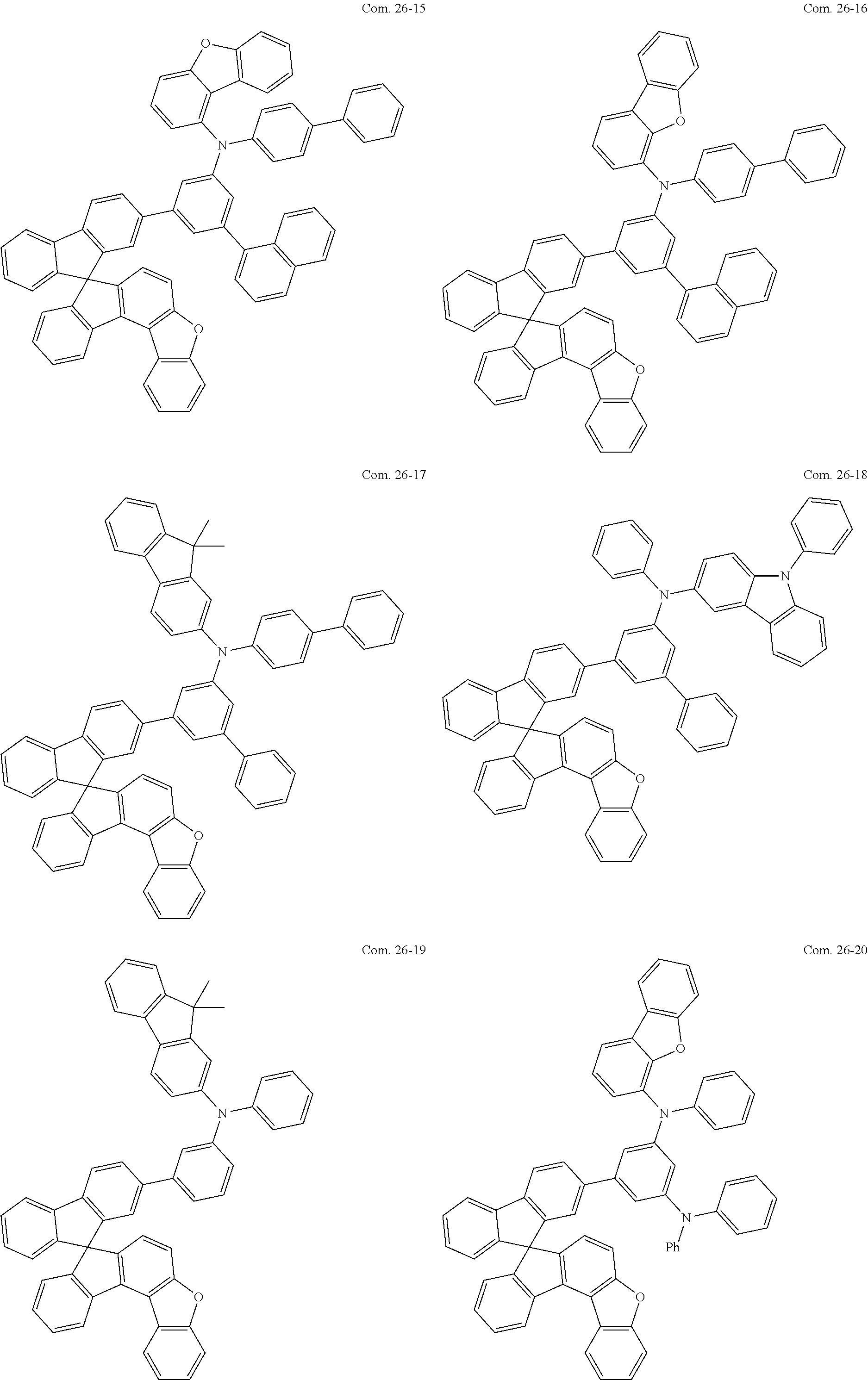

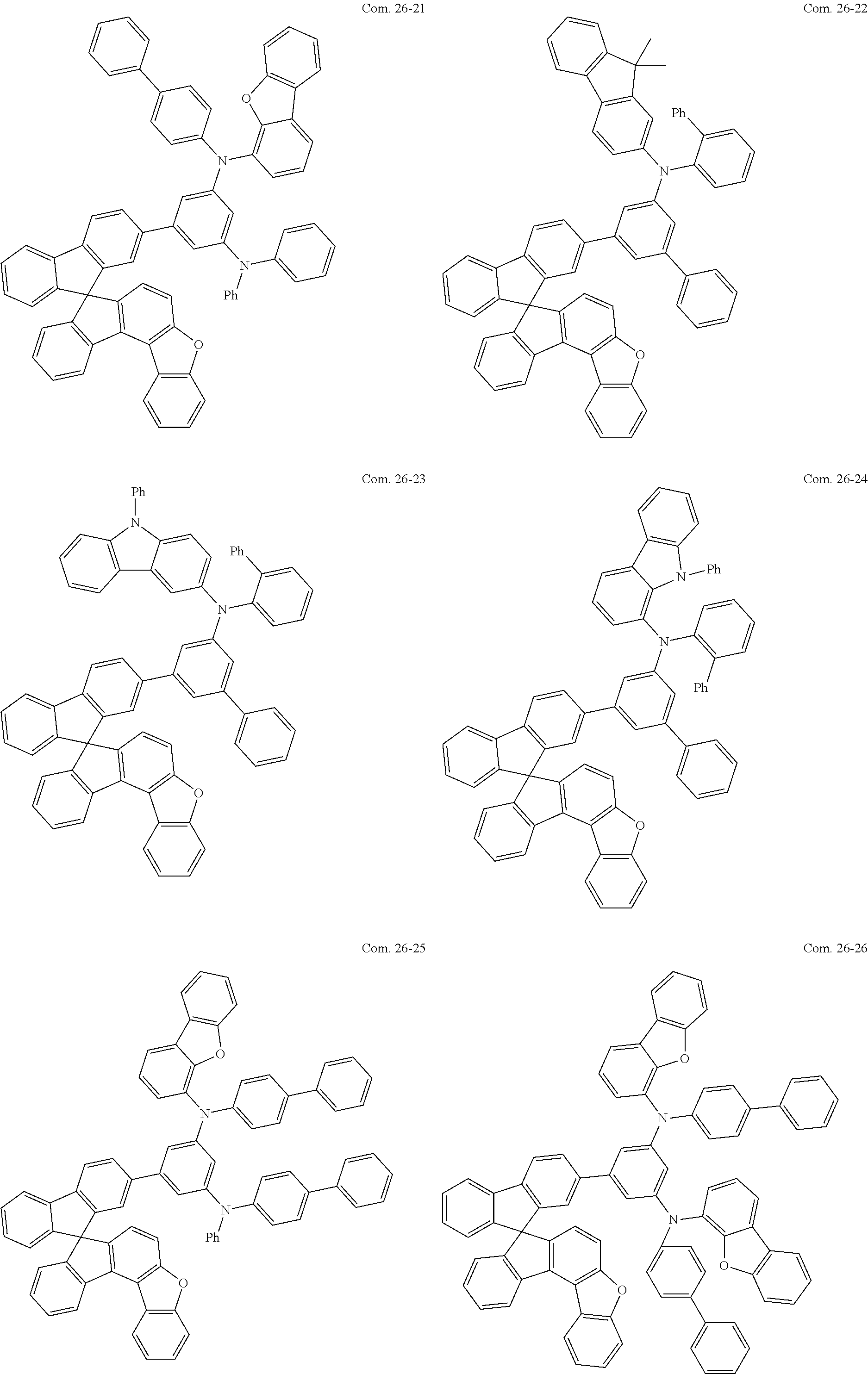

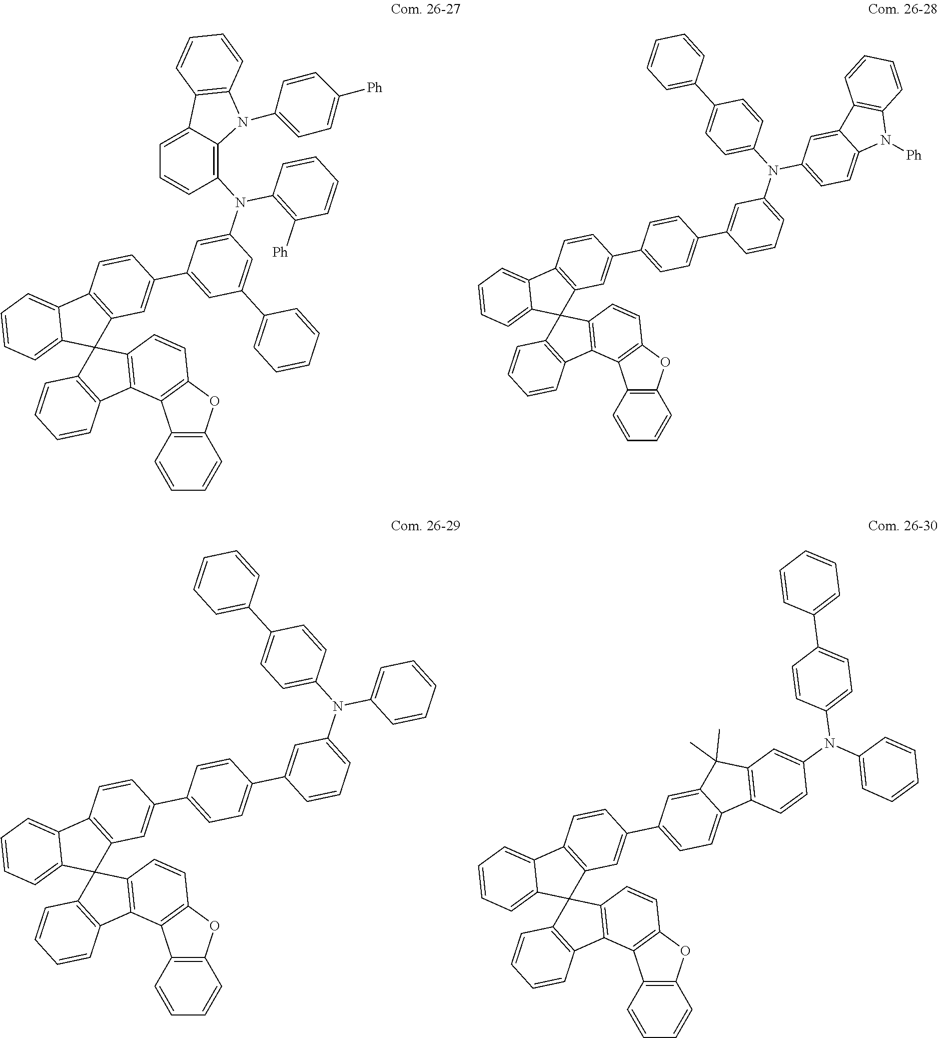 [SMILES: c1ccc(-c2ccc(-n3c4ccccc4c4cccc(N(c5cc(-c6ccccc6)cc(-c6ccc7c(c6)C6(c8ccccc8-7)c7ccccc7-c7c6ccc6oc8ccccc8c76)c5)c5ccccc5-c5ccccc5)c43)cc2)cc1 ; c1ccc(-c2ccc(N(c3cccc(-c4ccc(-c5ccc6c(c5)C5(c7ccccc7-6)c6ccccc6-c6c5ccc5oc7ccccc7c65)cc4)c3)c3ccc4c(c3)c3ccccc3n4-c3ccccc3)cc2)cc1 ; c1ccc(-c2ccc(N(c3ccccc3)c3cccc(-c4ccc(-c5ccc6c(c5)C5(c7ccccc7-6)c6ccccc6-c6c5ccc5oc7ccccc7c65)cc4)c3)cc2)cc1 ; CC1(C)c2cc(-c3ccc4c(c3)C3(c5ccccc5-4)c4ccccc4-c4c3ccc3oc5ccccc5c43)ccc2-c2ccc(N(c3ccccc3)c3ccc(-c4ccccc4)cc3)cc21]

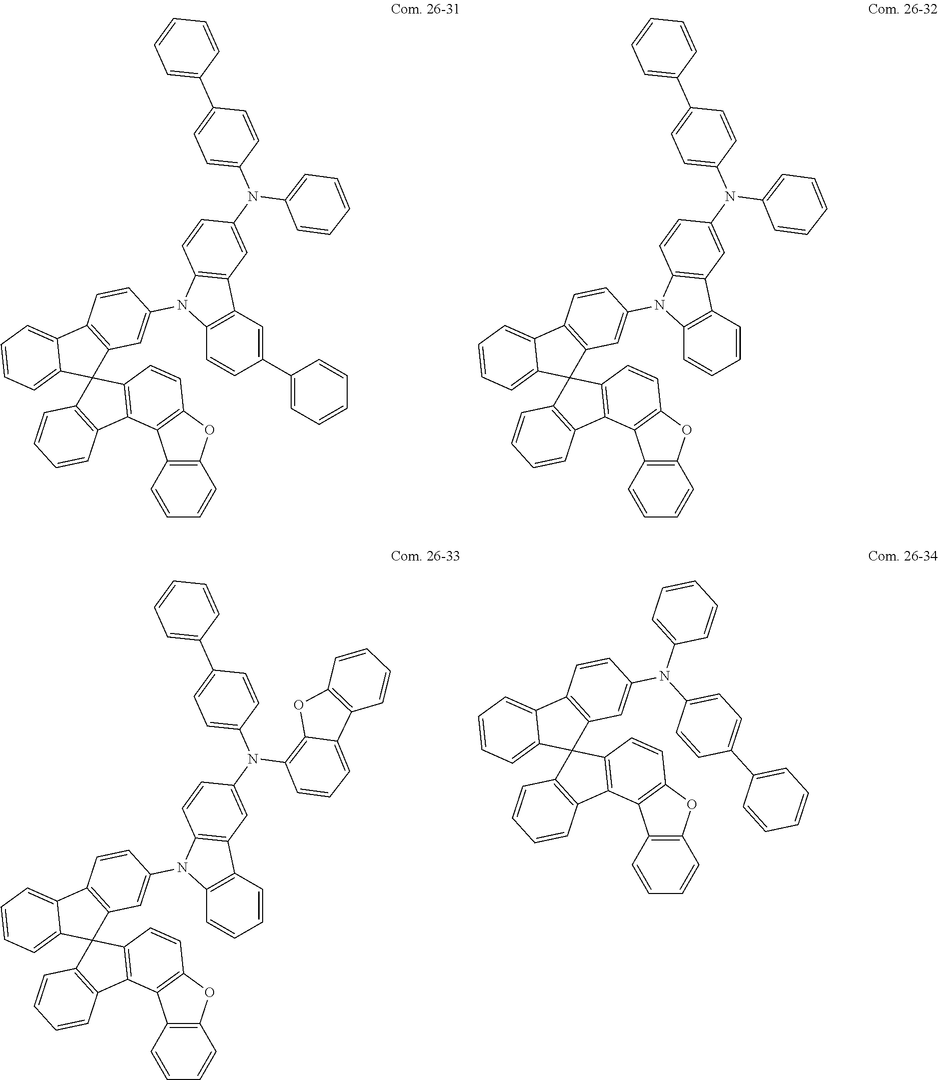 [SMILES: c1ccc(-c2ccc(N(c3ccccc3)c3ccc4c(c3)c3cc(-c5ccccc5)ccc3n4-c3ccc4c(c3)C3(c5ccccc5-4)c4ccccc4-c4c3ccc3oc5ccccc5c43)cc2)cc1 ; c1ccc(-c2ccc(N(c3ccccc3)c3ccc4c(c3)c3ccccc3n4-c3ccc4c(c3)C3(c5ccccc5-4)c4ccccc4-c4c3ccc3oc5ccccc5c43)cc2)cc1 ; c1ccc(-c2ccc(N(c3ccc4c(c3)c3ccccc3n4-c3ccc4c(c3)C3(c5ccccc5-4)c4ccccc4-c4c3ccc3oc5ccccc5c43)c3cccc4c3oc3ccccc34)cc2)cc1 ; c1ccc(-c2ccc(N(c3ccccc3)c3ccc4c(c3)C3(c5ccccc5-4)c4ccccc4-c4c3ccc3oc5ccccc5c43)cc2)cc1]

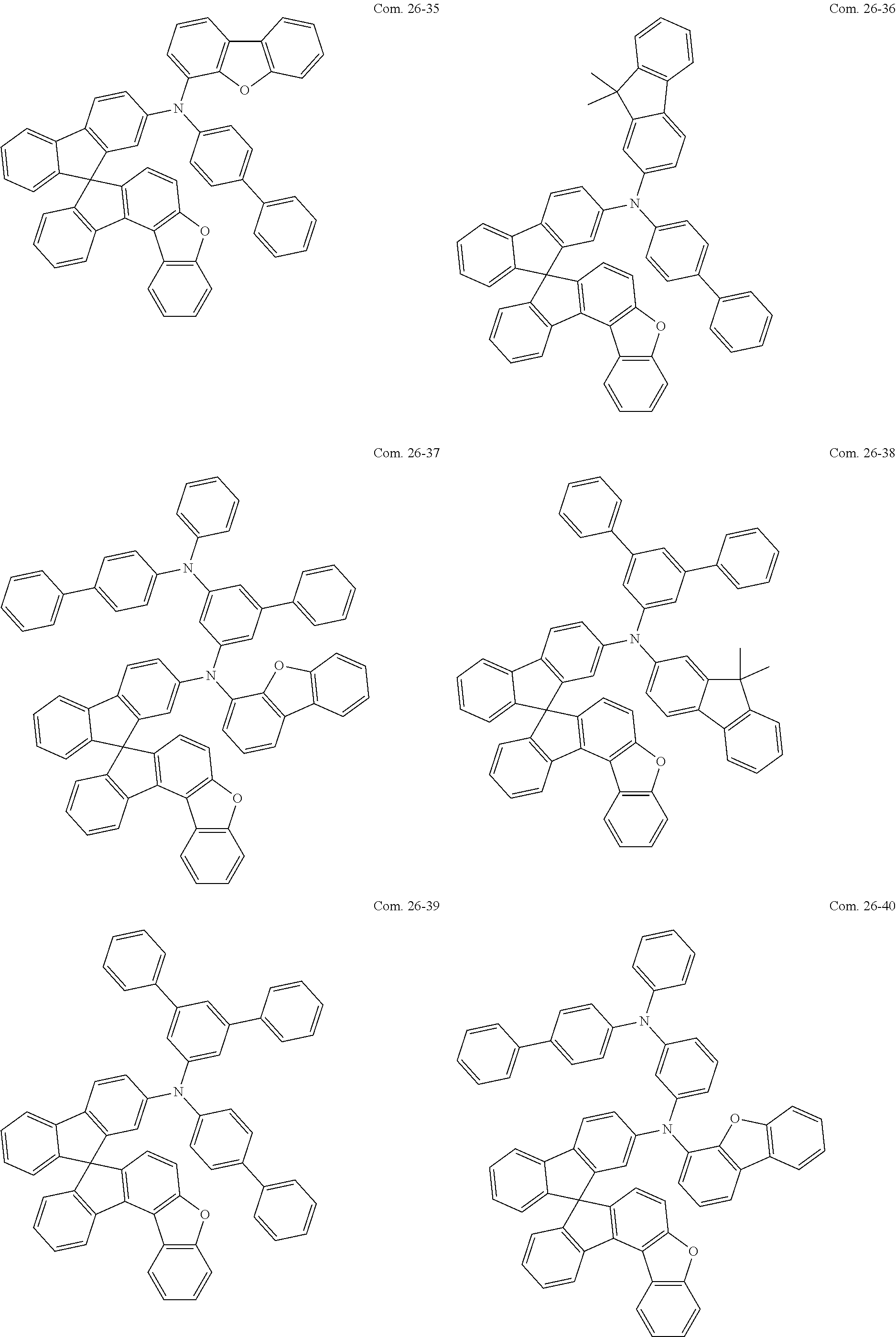

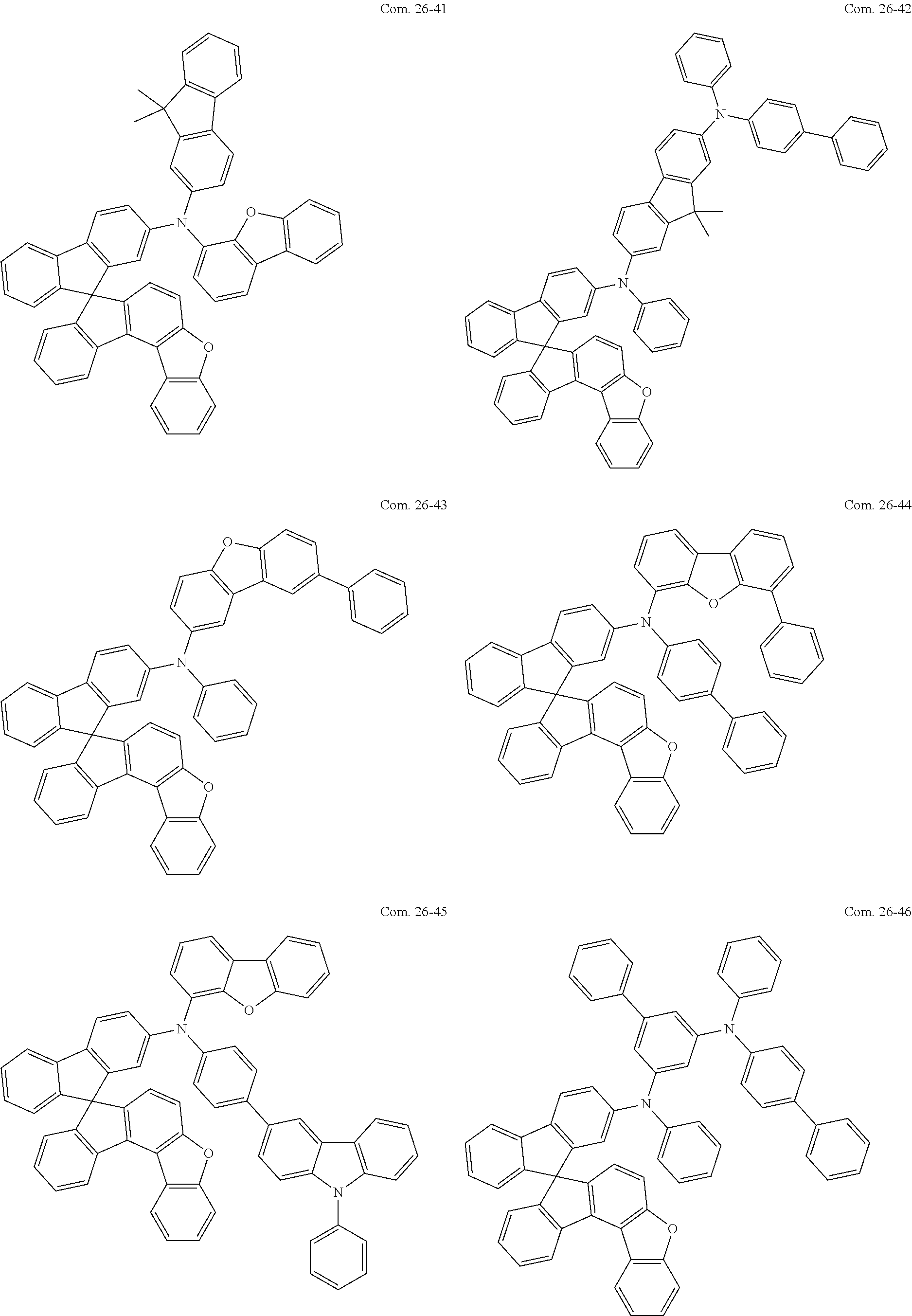

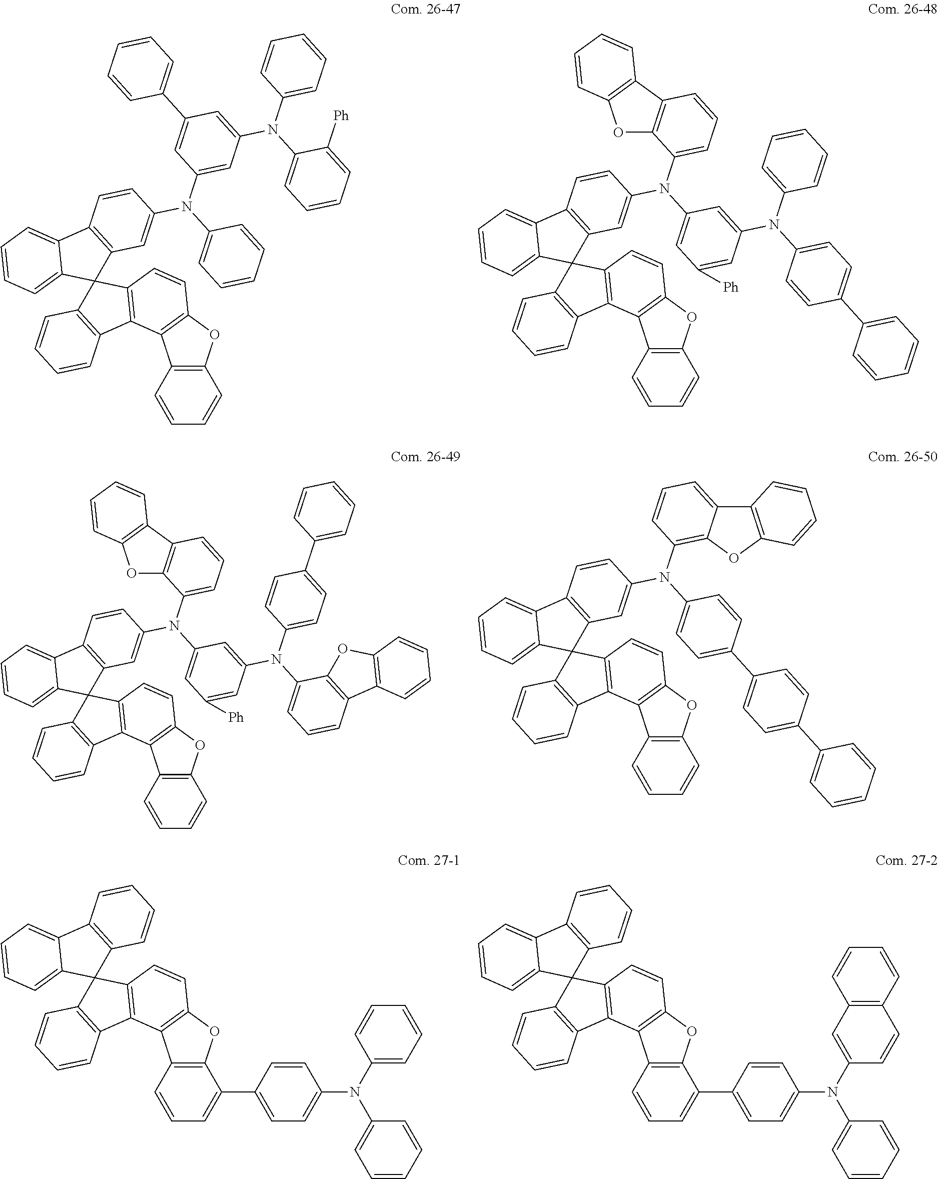

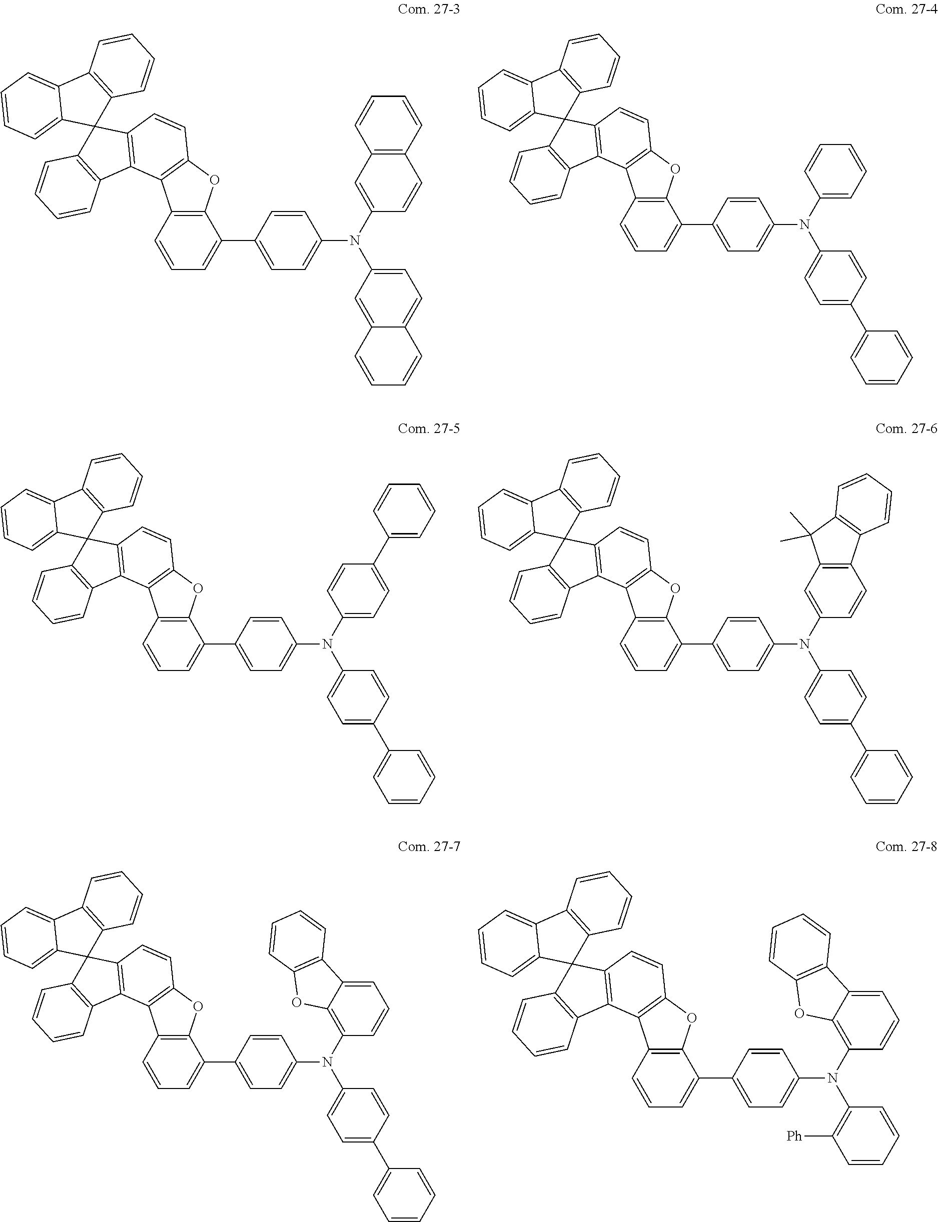

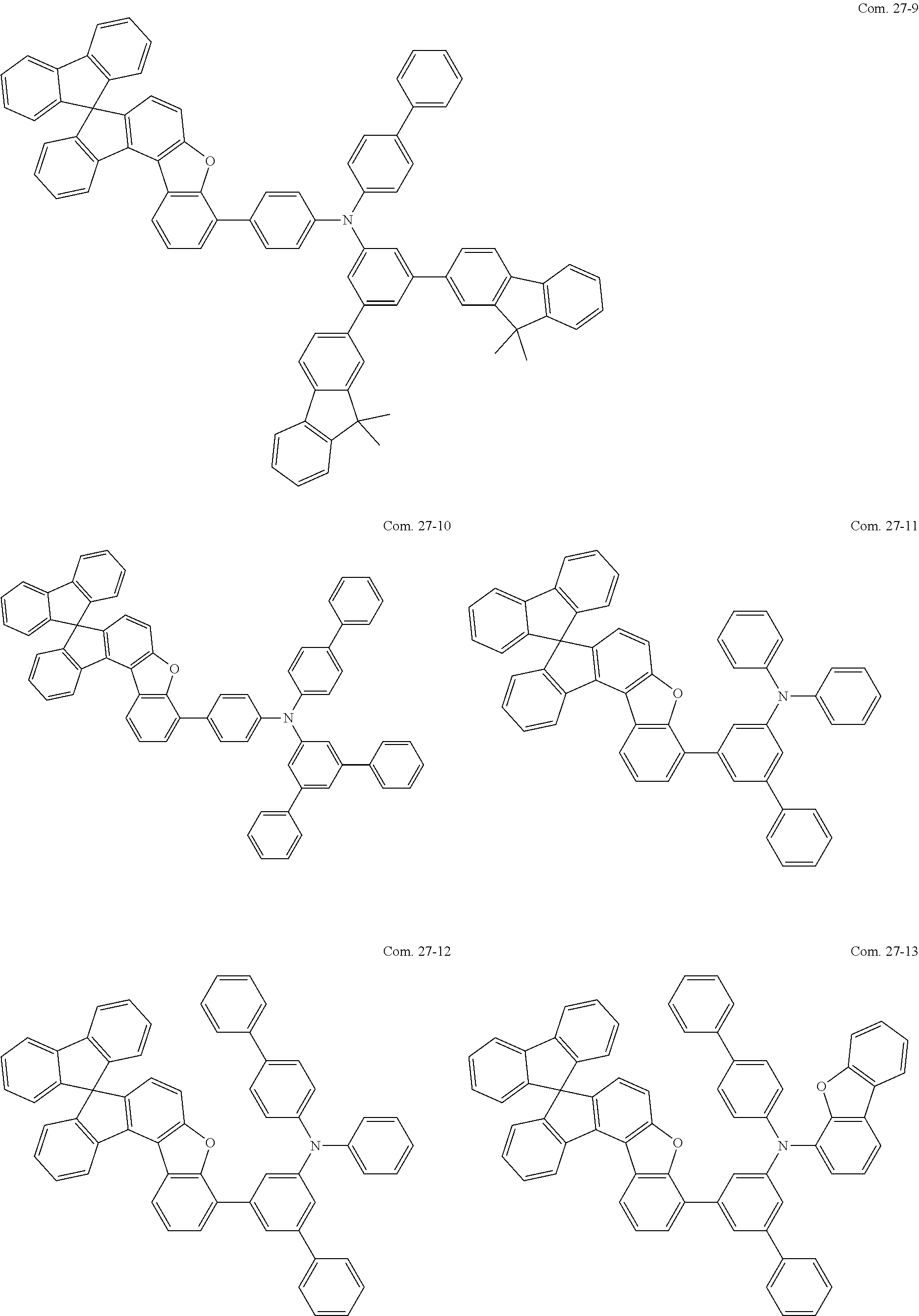

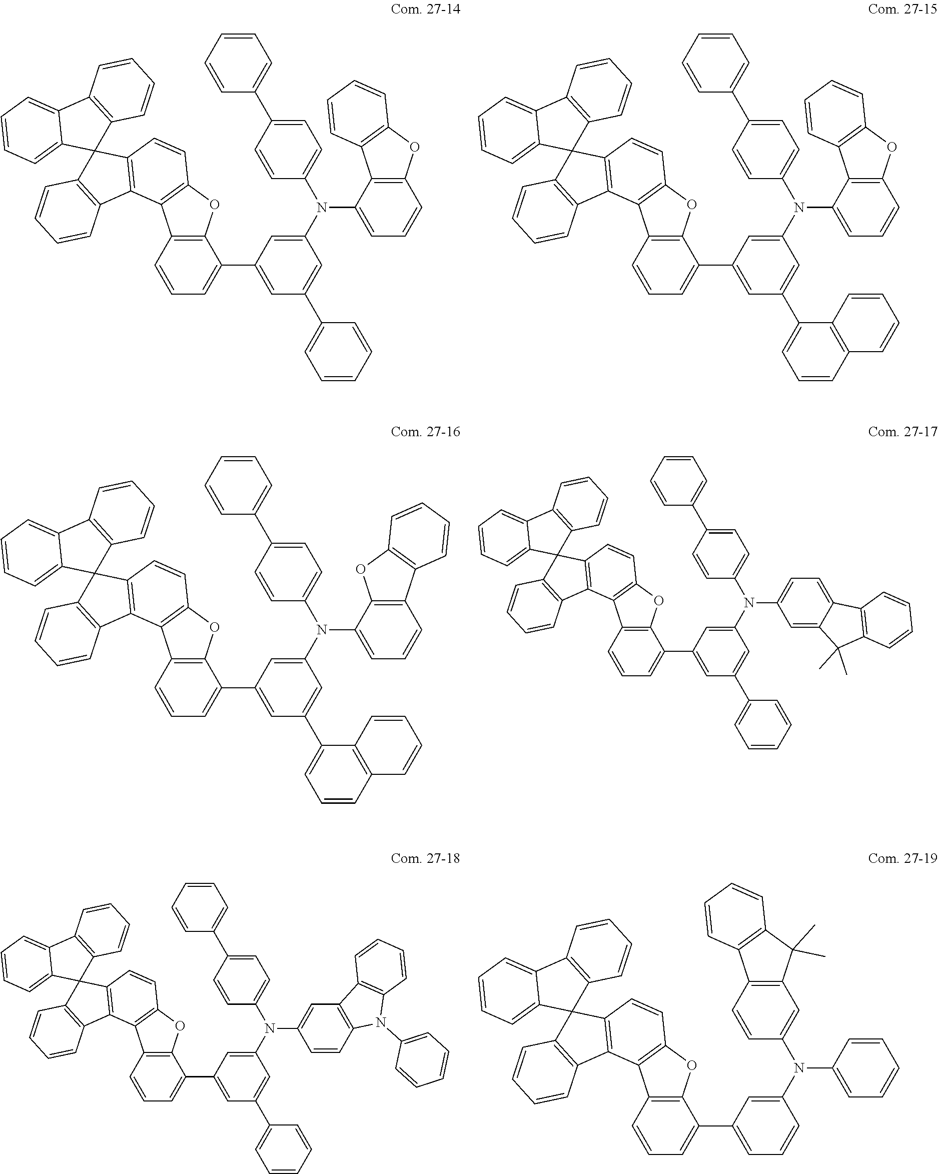

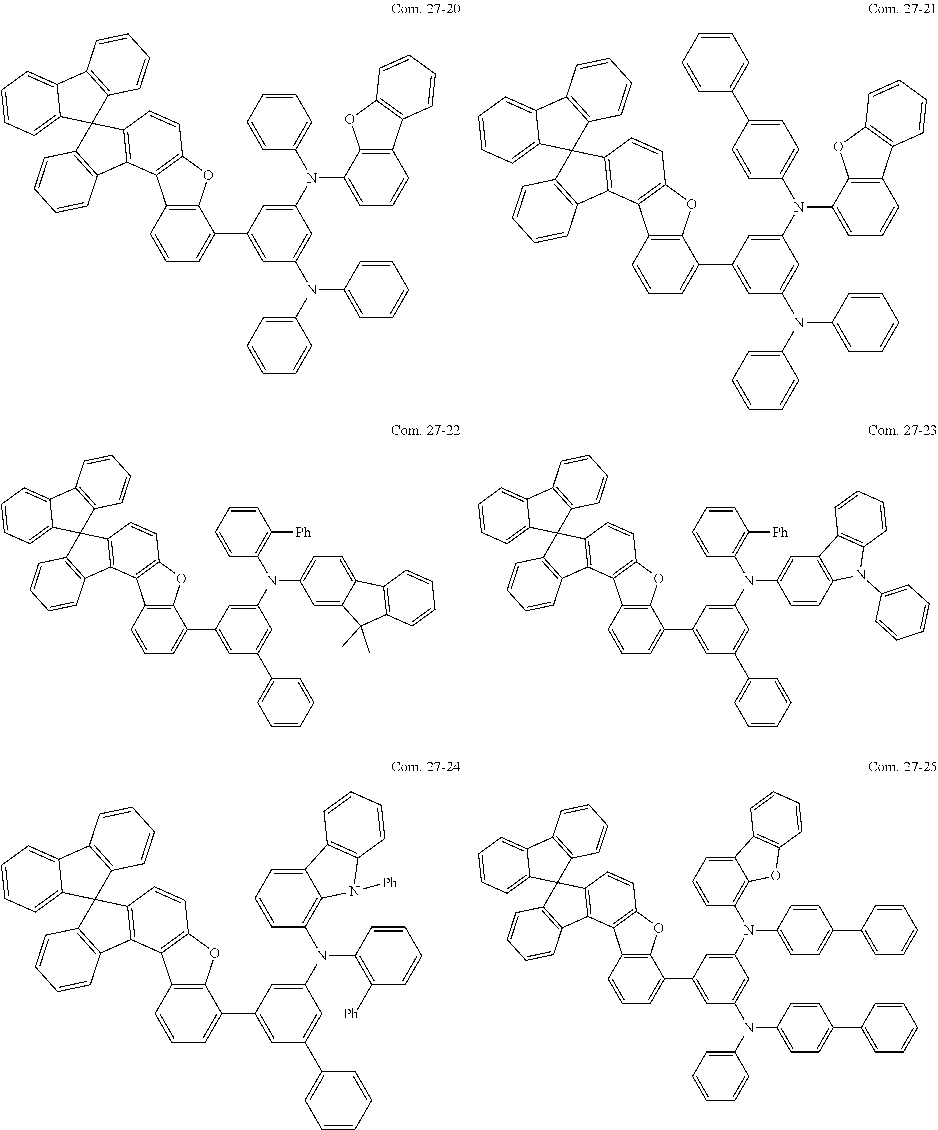

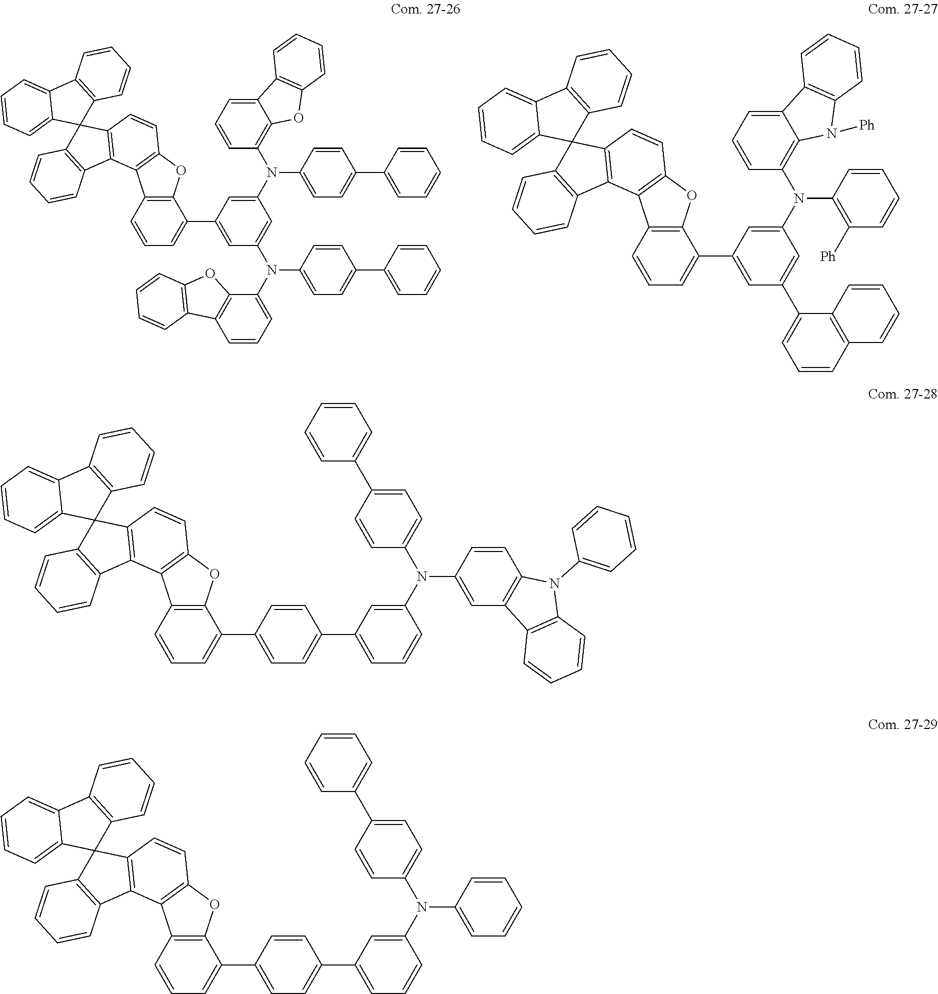

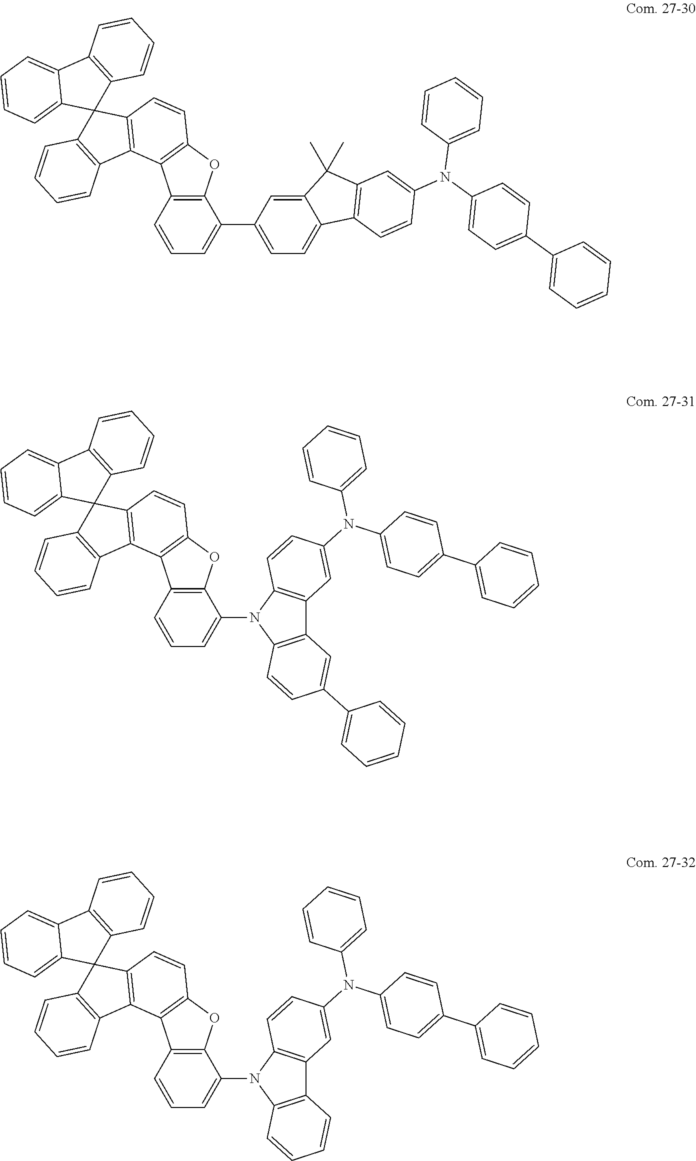 [SMILES: CC1(C)c2cc(-c3cccc4c3oc3ccc5c(c34)-c3ccccc3C53c4ccccc4-c4ccccc43)ccc2-c2ccc(N(c3ccccc3)c3ccc(-c4ccccc4)cc3)cc21 ; c1ccc(-c2ccc(N(c3ccccc3)c3ccc4c(c3)c3cc(-c5ccccc5)ccc3n4-c3cccc4c3oc3ccc5c(c34)-c3ccccc3C53c4ccccc4-c4ccccc43)cc2)cc1 ; c1ccc(-c2ccc(N(c3ccccc3)c3ccc4c(c3)c3ccccc3n4-c3cccc4c3oc3ccc5c(c34)-c3ccccc3C53c4ccccc4-c4ccccc43)cc2)cc1]

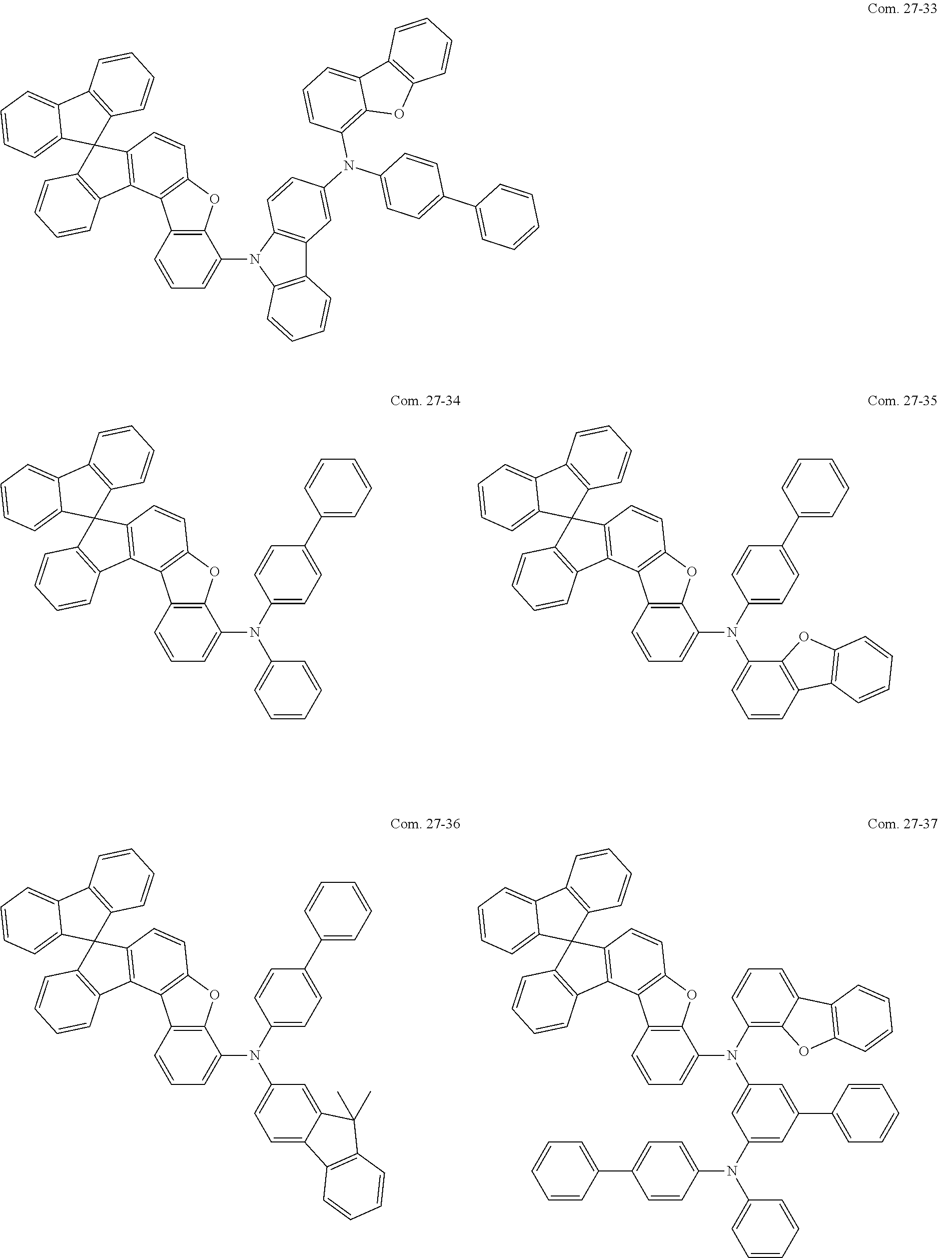

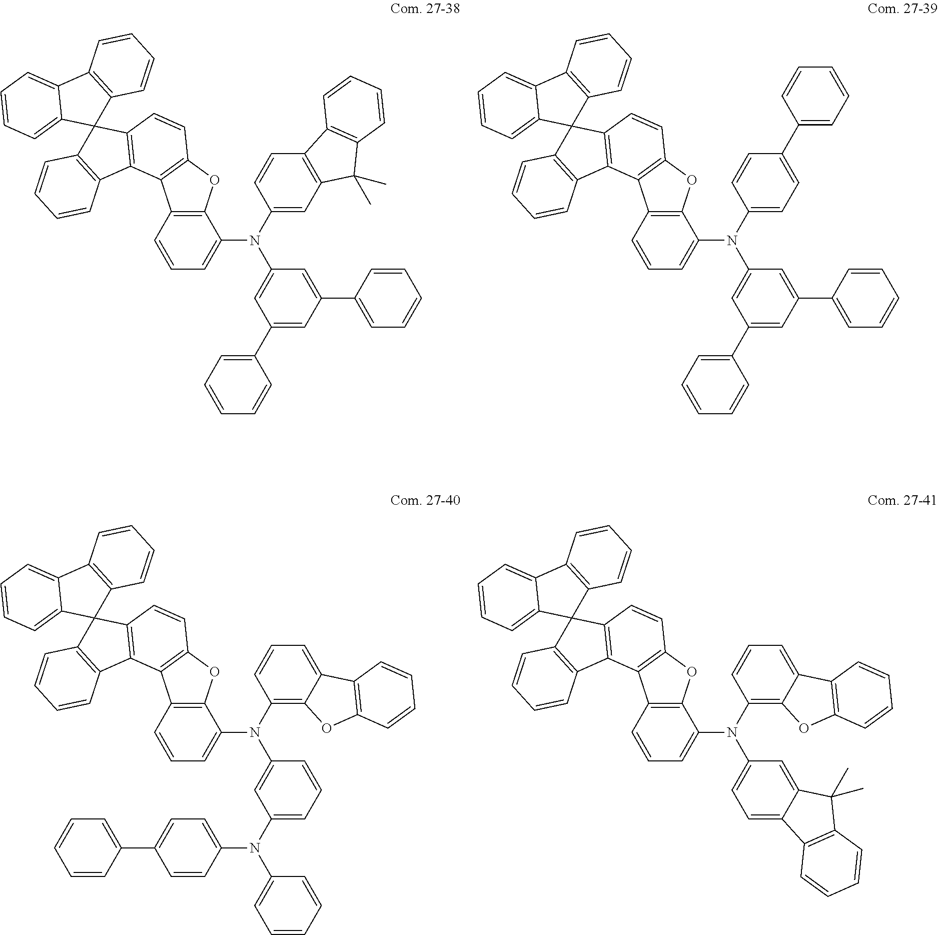 [SMILES: CC1(C)c2ccccc2-c2ccc(N(c3cc(-c4ccccc4)cc(-c4ccccc4)c3)c3cccc4c3oc3ccc5c(c34)-c3ccccc3C53c4ccccc4-c4ccccc43)cc21 ; c1ccc(-c2ccc(N(c3cc(-c4ccccc4)cc(-c4ccccc4)c3)c3cccc4c3oc3ccc5c(c34)-c3ccccc3C53c4ccccc4-c4ccccc43)cc2)cc1 ; c1ccc(-c2ccc(N(c3ccccc3)c3cccc(N(c4cccc5c4oc4ccccc45)c4cccc5c4oc4ccc6c(c45)-c4ccccc4C64c5ccccc5-c5ccccc54)c3)cc2)cc1 ; CC1(C)c2ccccc2-c2ccc(N(c3cccc4c3oc3ccccc34)c3cccc4c3oc3ccc5c(c34)-c3ccccc3C53c4ccccc4-c4ccccc43)cc21]

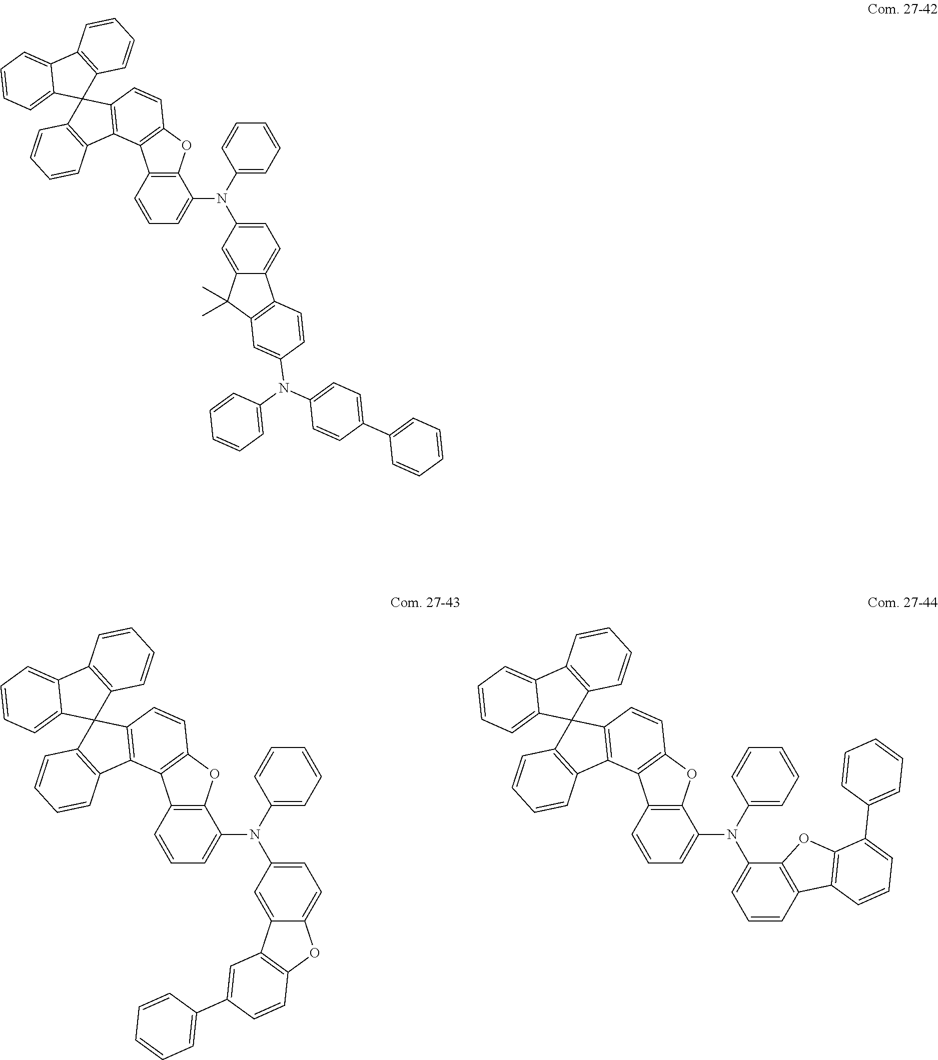 [SMILES: CC1(C)c2cc(N(c3ccccc3)c3ccc(-c4ccccc4)cc3)ccc2-c2ccc(N(c3ccccc3)c3cccc4c3oc3ccc5c(c34)-c3ccccc3C53c4ccccc4-c4ccccc43)cc21 ; c1ccc(-c2ccc3oc4ccc(N(c5ccccc5)c5cccc6c5oc5ccc7c(c56)-c5ccccc5C75c6ccccc6-c6ccccc65)cc4c3c2)cc1 ; c1ccc(-c2cccc3c2oc2c(N(c4ccccc4)c4cccc5c4oc4ccc6c(c45)-c4ccccc4C64c5ccccc5-c5ccccc54)cccc23)cc1]

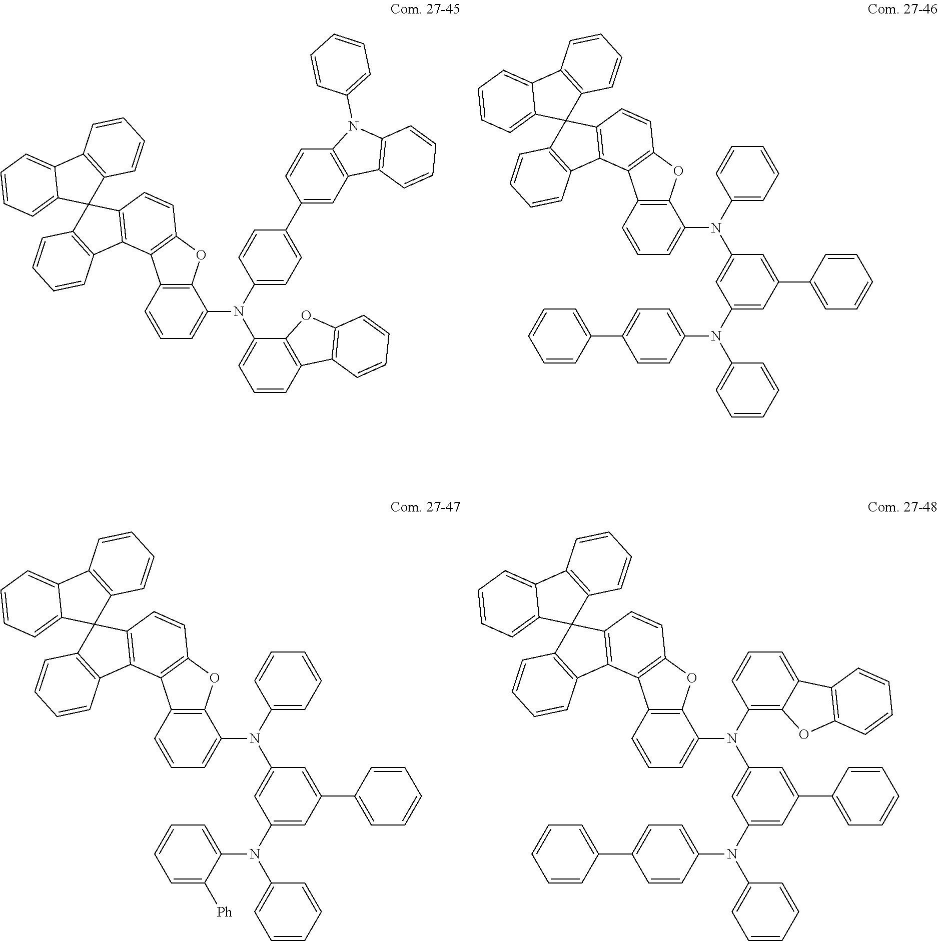 [SMILES: c1ccc(-n2c3ccccc3c3cc(-c4ccc(N(c5cccc6c5oc5ccccc56)c5cccc6c5oc5ccc7c(c56)-c5ccccc5C75c6ccccc6-c6ccccc65)cc4)ccc32)cc1 ; c1ccc(-c2ccc(N(c3ccccc3)c3cc(-c4ccccc4)cc(N(c4ccccc4)c4cccc5c4oc4ccc6c(c45)-c4ccccc4C64c5ccccc5-c5ccccc54)c3)cc2)cc1 ; c1ccc(-c2cc(N(c3ccccc3)c3ccccc3-c3ccccc3)cc(N(c3ccccc3)c3cccc4c3oc3ccc5c(c34)-c3ccccc3C53c4ccccc4-c4ccccc43)c2)cc1 ; c1ccc(-c2ccc(N(c3ccccc3)c3cc(-c4ccccc4)cc(N(c4cccc5c4oc4ccccc45)c4cccc5c4oc4ccc6c(c45)-c4ccccc4C64c5ccccc5-c5ccccc54)c3)cc2)cc1]

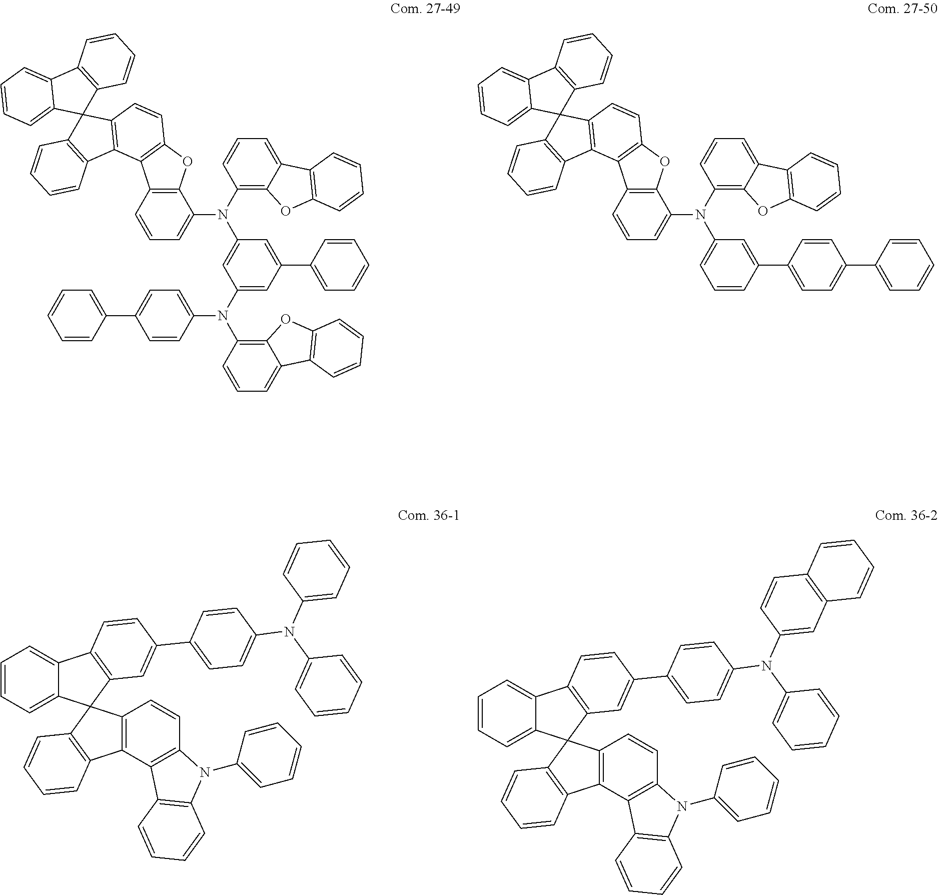 [SMILES: c1ccc(-c2ccc(N(c3cc(-c4ccccc4)cc(N(c4cccc5c4oc4ccccc45)c4cccc5c4oc4ccc6c(c45)-c4ccccc4C64c5ccccc5-c5ccccc54)c3)c3cccc4c3oc3ccccc34)cc2)cc1 ; c1ccc(-c2ccc(-c3cccc(N(c4cccc5c4oc4ccccc45)c4cccc5c4oc4ccc6c(c45)-c4ccccc4C64c5ccccc5-c5ccccc54)c3)cc2)cc1 ; c1ccc(N(c2ccccc2)c2ccc(-c3ccc4c(c3)C3(c5ccccc5-4)c4ccccc4-c4c3ccc3c4c4ccccc4n3-c3ccccc3)cc2)cc1 ; c1ccc(N(c2ccc(-c3ccc4c(c3)C3(c5ccccc5-4)c4ccccc4-c4c3ccc3c4c4ccccc4n3-c3ccccc3)cc2)c2ccc3ccccc3c2)cc1]

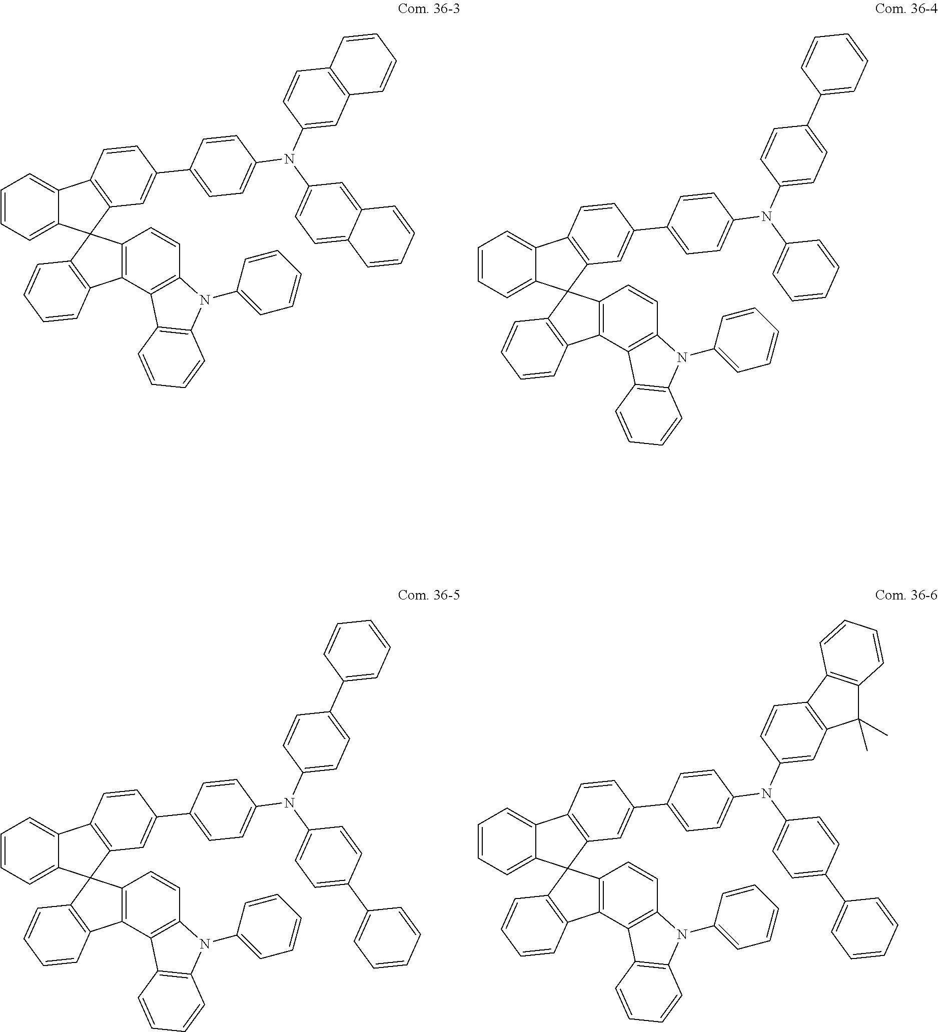 [SMILES: c1ccc(-n2c3ccccc3c3c4c(ccc32)C2(c3ccccc3-c3ccc(-c5ccc(N(c6ccc7ccccc7c6)c6ccc7ccccc7c6)cc5)cc32)c2ccccc2-4)cc1 ; c1ccc(-c2ccc(N(c3ccccc3)c3ccc(-c4ccc5c(c4)C4(c6ccccc6-5)c5ccccc5-c5c4ccc4c5c5ccccc5n4-c4ccccc4)cc3)cc2)cc1 ; c1ccc(-c2ccc(N(c3ccc(-c4ccccc4)cc3)c3ccc(-c4ccc5c(c4)C4(c6ccccc6-5)c5ccccc5-c5c4ccc4c5c5ccccc5n4-c4ccccc4)cc3)cc2)cc1 ; CC1(C)c2ccccc2-c2ccc(N(c3ccc(-c4ccccc4)cc3)c3ccc(-c4ccc5c(c4)C4(c6ccccc6-5)c5ccccc5-c5c4ccc4c5c5ccccc5n4-c4ccccc4)cc3)cc21]

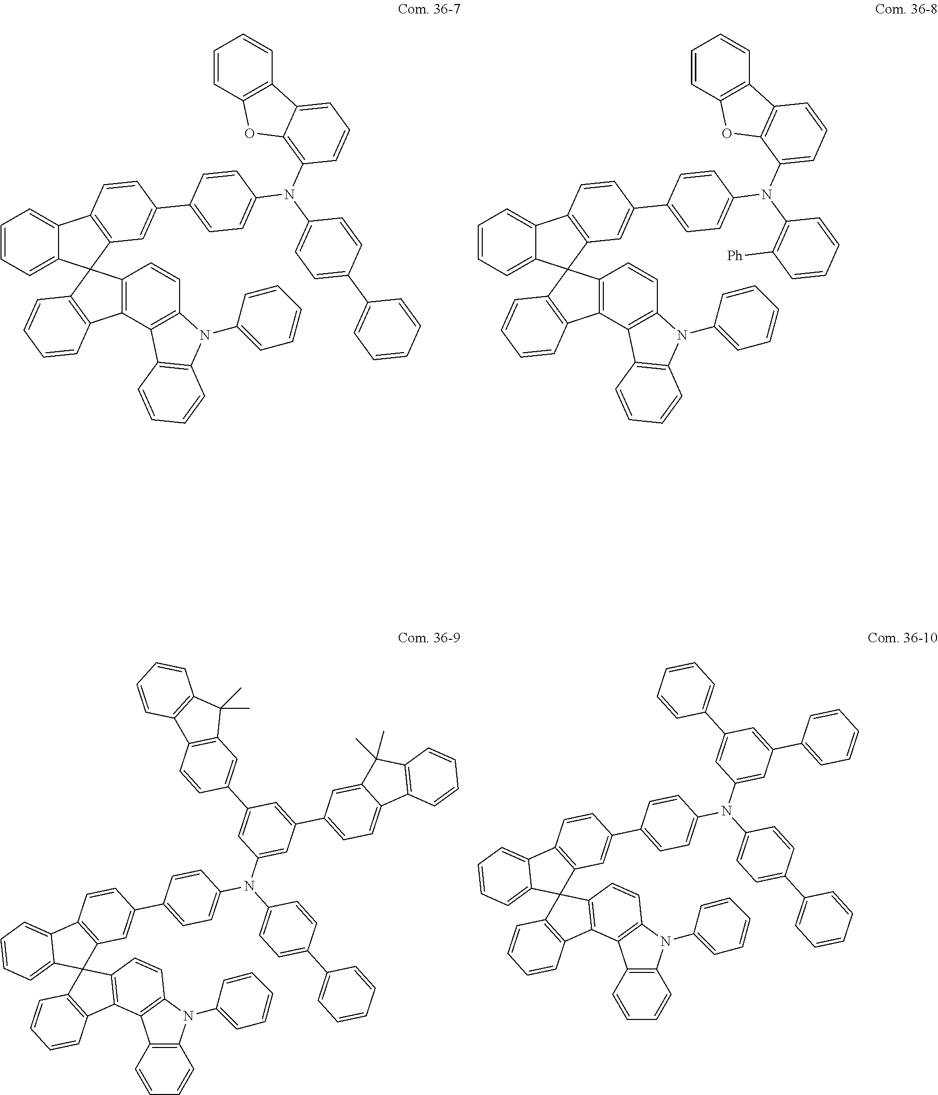

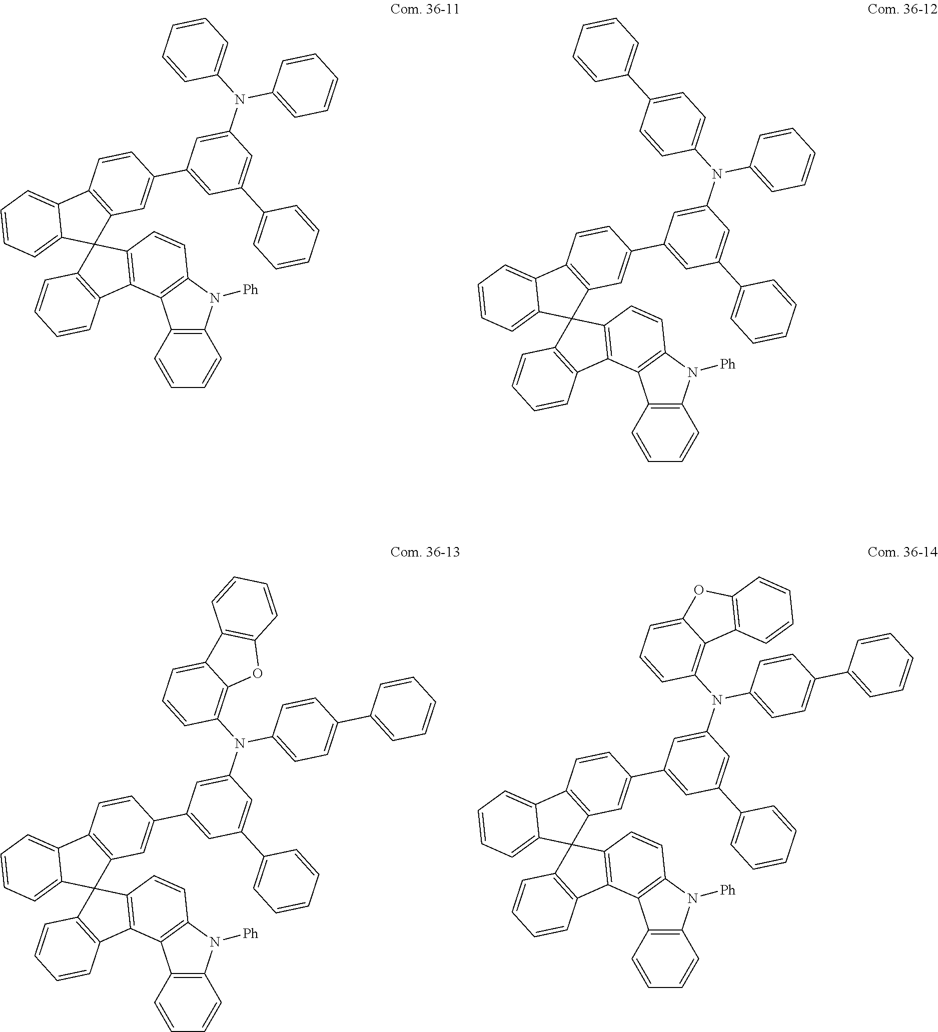 [SMILES: c1ccc(-c2cc(-c3ccc4c(c3)C3(c5ccccc5-4)c4ccccc4-c4c3ccc3c4c4ccccc4n3-c3ccccc3)cc(N(c3ccccc3)c3ccccc3)c2)cc1 ; c1ccc(-c2ccc(N(c3ccccc3)c3cc(-c4ccccc4)cc(-c4ccc5c(c4)C4(c6ccccc6-5)c5ccccc5-c5c4ccc4c5c5ccccc5n4-c4ccccc4)c3)cc2)cc1 ; c1ccc(-c2ccc(N(c3cc(-c4ccccc4)cc(-c4ccc5c(c4)C4(c6ccccc6-5)c5ccccc5-c5c4ccc4c5c5ccccc5n4-c4ccccc4)c3)c3cccc4c3oc3ccccc34)cc2)cc1 ; c1ccc(-c2ccc(N(c3cc(-c4ccccc4)cc(-c4ccc5c(c4)C4(c6ccccc6-5)c5ccccc5-c5c4ccc4c5c5ccccc5n4-c4ccccc4)c3)c3cccc4oc5ccccc5c34)cc2)cc1]

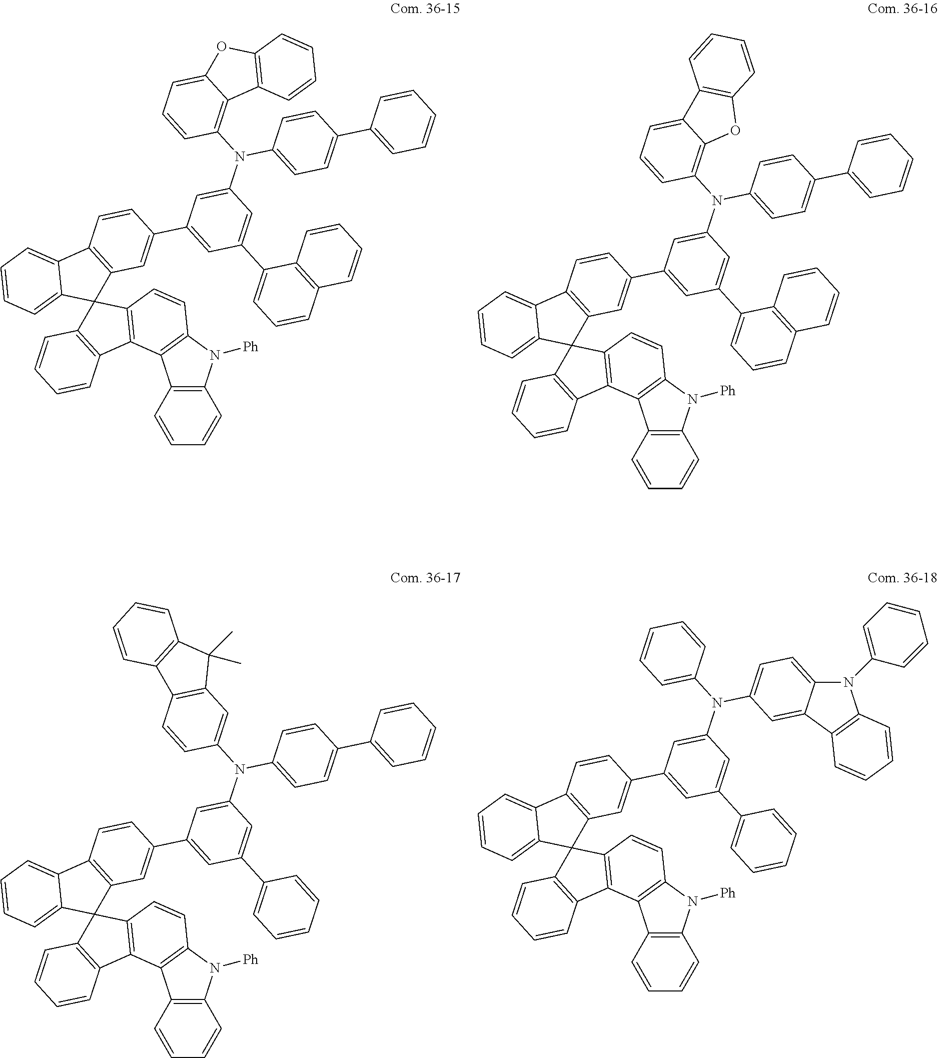

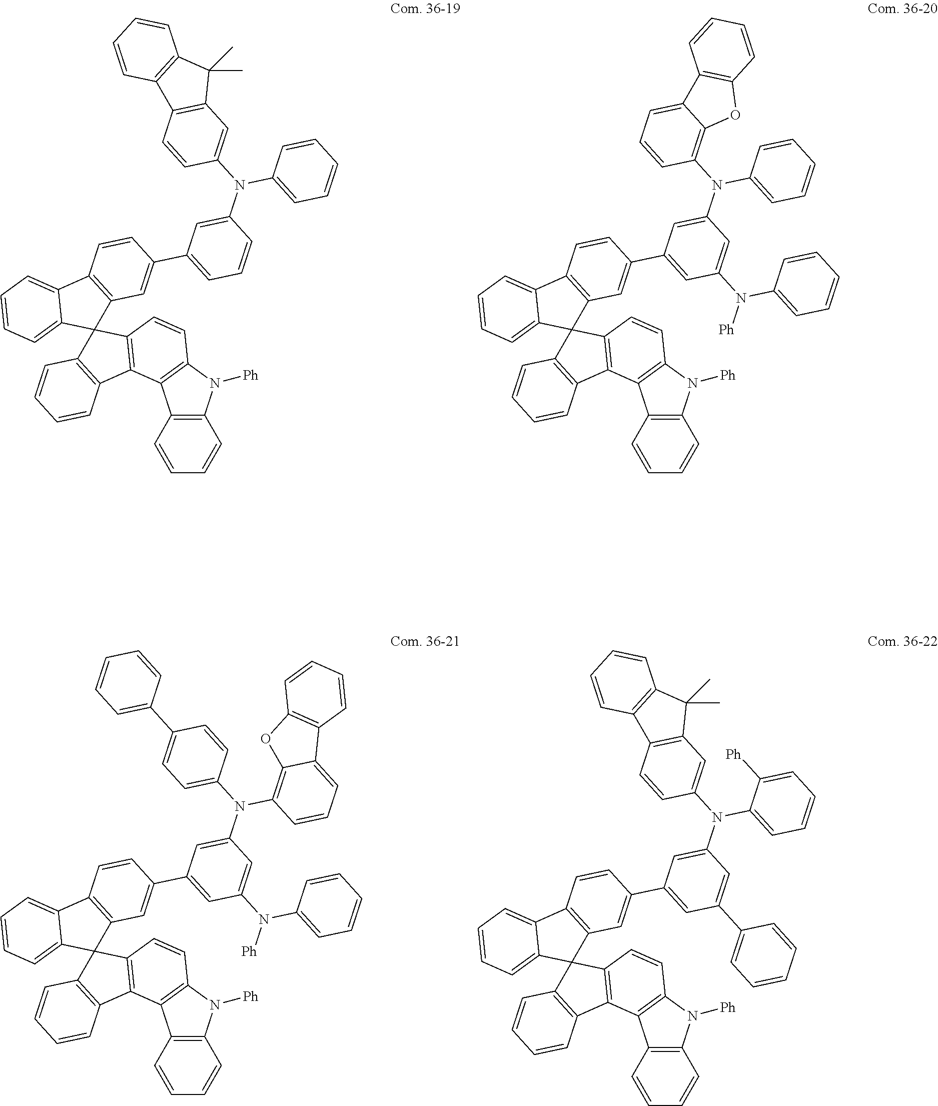

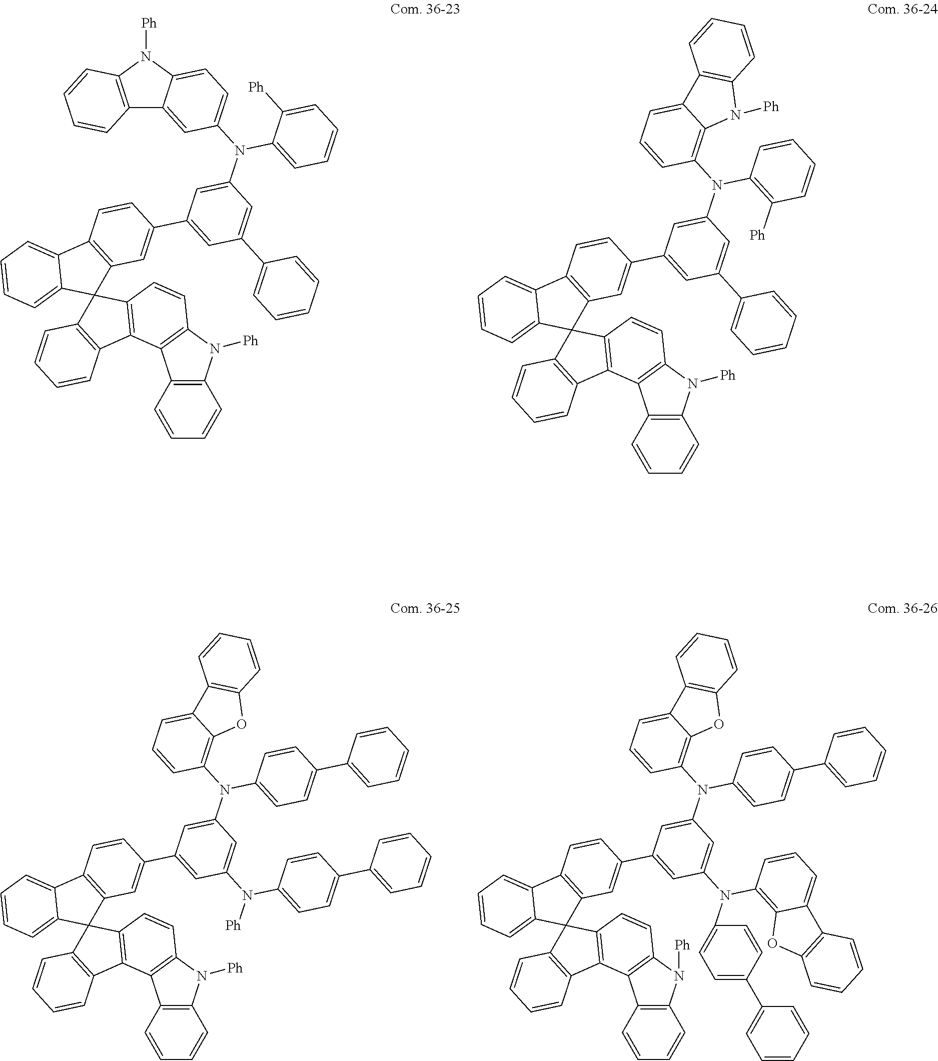

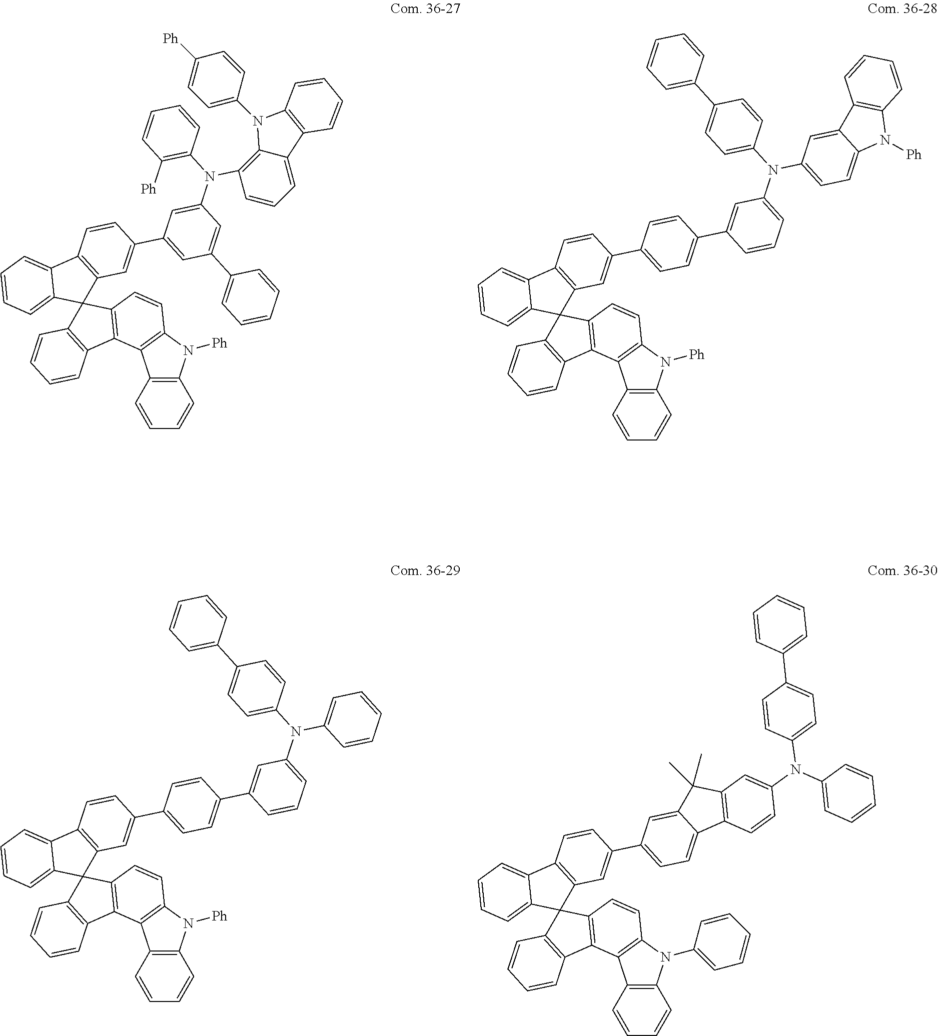

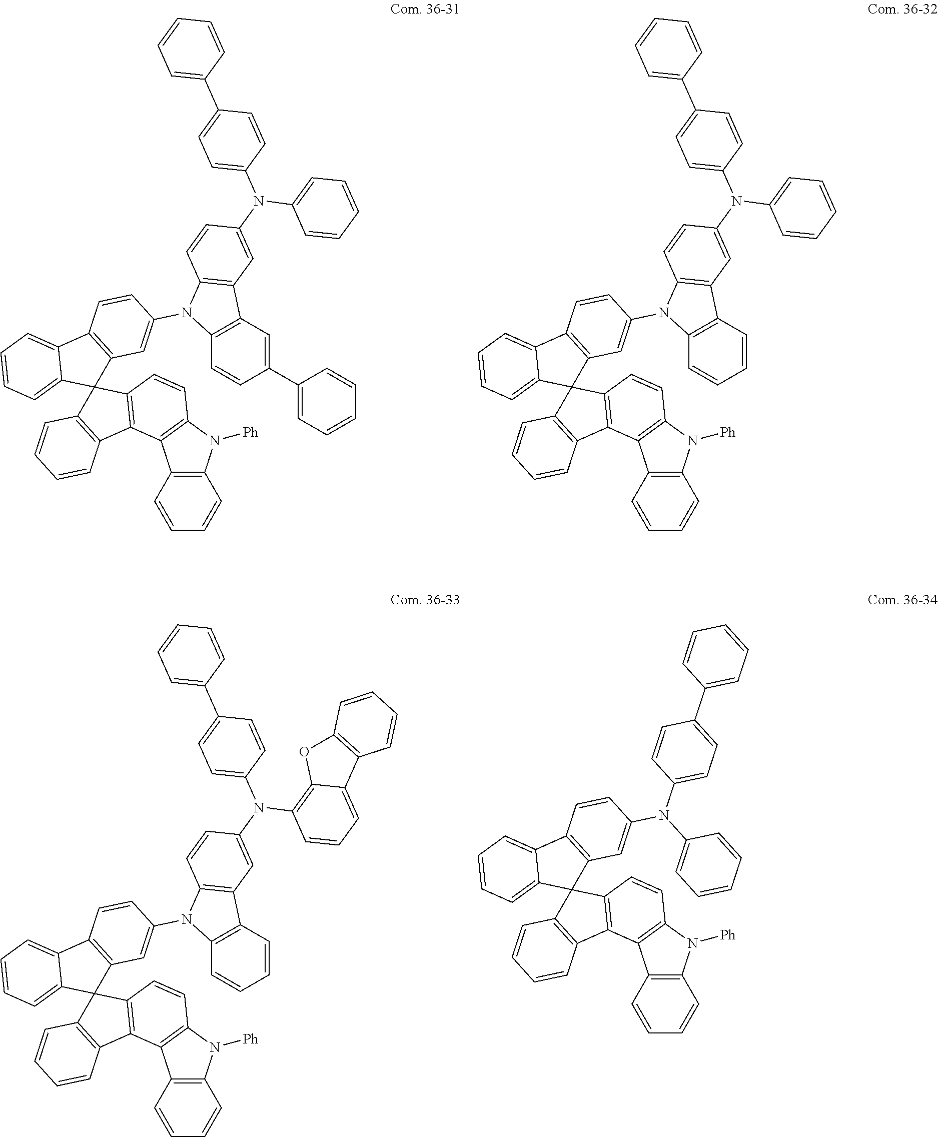 [SMILES: c1ccc(-c2ccc(N(c3ccccc3)c3ccc4c(c3)c3cc(-c5ccccc5)ccc3n4-c3ccc4c(c3)C3(c5ccccc5-4)c4ccccc4-c4c3ccc3c4c4ccccc4n3-c3ccccc3)cc2)cc1 ; c1ccc(-c2ccc(N(c3ccccc3)c3ccc4c(c3)c3ccccc3n4-c3ccc4c(c3)C3(c5ccccc5-4)c4ccccc4-c4c3ccc3c4c4ccccc4n3-c3ccccc3)cc2)cc1 ; c1ccc(-c2ccc(N(c3ccc4c(c3)c3ccccc3n4-c3ccc4c(c3)C3(c5ccccc5-4)c4ccccc4-c4c3ccc3c4c4ccccc4n3-c3ccccc3)c3cccc4c3oc3ccccc34)cc2)cc1 ; c1ccc(-c2ccc(N(c3ccccc3)c3ccc4c(c3)C3(c5ccccc5-4)c4ccccc4-c4c3ccc3c4c4ccccc4n3-c3ccccc3)cc2)cc1]

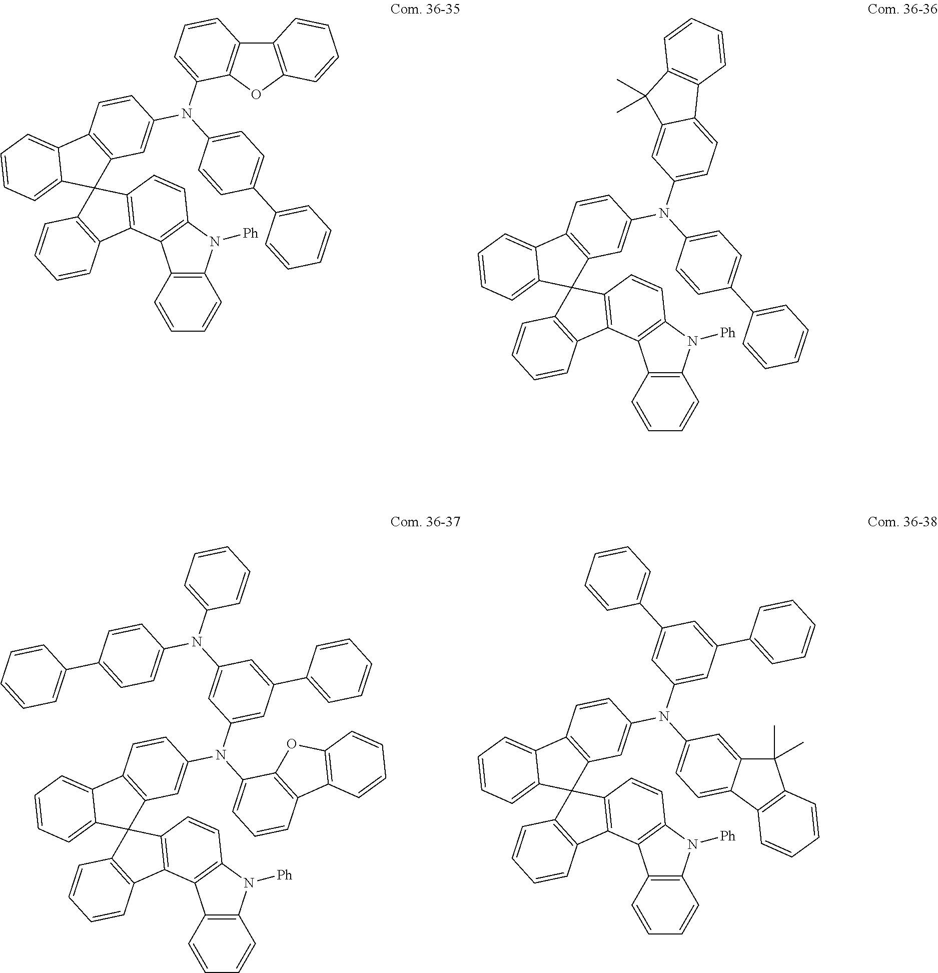 [SMILES: c1ccc(-c2ccc(N(c3ccc4c(c3)C3(c5ccccc5-4)c4ccccc4-c4c3ccc3c4c4ccccc4n3-c3ccccc3)c3cccc4c3oc3ccccc34)cc2)cc1 ; CC1(C)c2ccccc2-c2ccc(N(c3ccc(-c4ccccc4)cc3)c3ccc4c(c3)C3(c5ccccc5-4)c4ccccc4-c4c3ccc3c4c4ccccc4n3-c3ccccc3)cc21 ; c1ccc(-c2ccc(N(c3ccccc3)c3cc(-c4ccccc4)cc(N(c4ccc5c(c4)C4(c6ccccc6-5)c5ccccc5-c5c4ccc4c5c5ccccc5n4-c4ccccc4)c4cccc5c4oc4ccccc45)c3)cc2)cc1 ; CC1(C)c2ccccc2-c2ccc(N(c3cc(-c4ccccc4)cc(-c4ccccc4)c3)c3ccc4c(c3)C3(c5ccccc5-4)c4ccccc4-c4c3ccc3c4c4ccccc4n3-c3ccccc3)cc21]

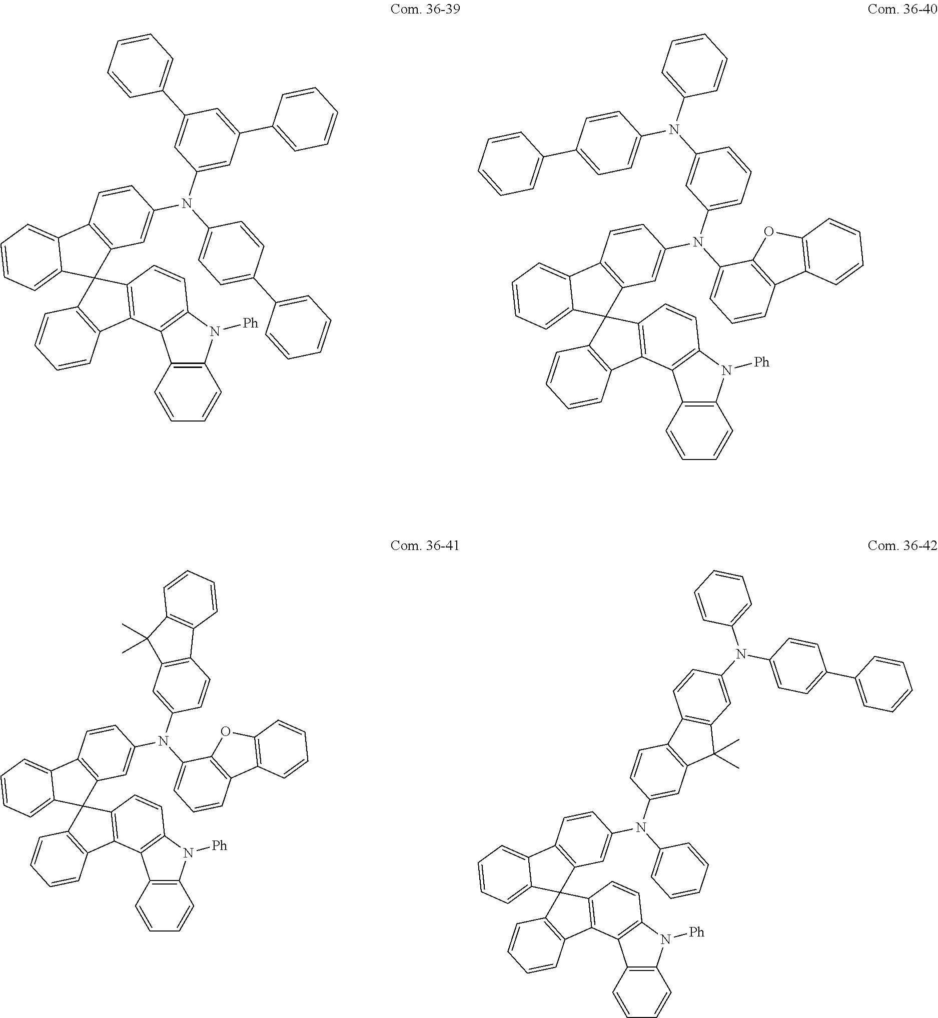 [SMILES: c1ccc(-c2ccc(N(c3cc(-c4ccccc4)cc(-c4ccccc4)c3)c3ccc4c(c3)C3(c5ccccc5-4)c4ccccc4-c4c3ccc3c4c4ccccc4n3-c3ccccc3)cc2)cc1 ; c1ccc(-c2ccc(N(c3ccccc3)c3cccc(N(c4ccc5c(c4)C4(c6ccccc6-5)c5ccccc5-c5c4ccc4c5c5ccccc5n4-c4ccccc4)c4cccc5c4oc4ccccc45)c3)cc2)cc1 ; CC1(C)c2ccccc2-c2ccc(N(c3ccc4c(c3)C3(c5ccccc5-4)c4ccccc4-c4c3ccc3c4c4ccccc4n3-c3ccccc3)c3cccc4c3oc3ccccc34)cc21 ; CC1(C)c2cc(N(c3ccccc3)c3ccc(-c4ccccc4)cc3)ccc2-c2ccc(N(c3ccccc3)c3ccc4c(c3)C3(c5ccccc5-4)c4ccccc4-c4c3ccc3c4c4ccccc4n3-c3ccccc3)cc21]

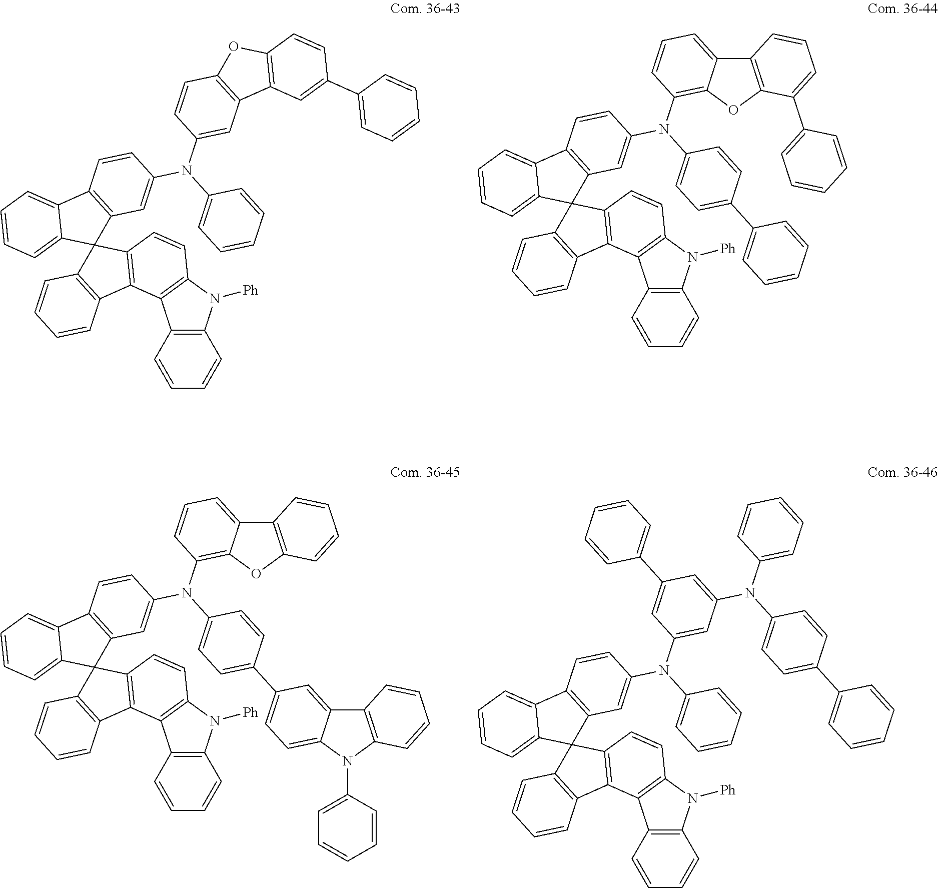 [SMILES: c1ccc(-c2ccc3oc4ccc(N(c5ccccc5)c5ccc6c(c5)C5(c7ccccc7-6)c6ccccc6-c6c5ccc5c6c6ccccc6n5-c5ccccc5)cc4c3c2)cc1 ; c1ccc(-c2ccc(N(c3ccc4c(c3)C3(c5ccccc5-4)c4ccccc4-c4c3ccc3c4c4ccccc4n3-c3ccccc3)c3cccc4c3oc3c(-c5ccccc5)cccc34)cc2)cc1 ; c1ccc(-n2c3ccccc3c3cc(-c4ccc(N(c5ccc6c(c5)C5(c7ccccc7-6)c6ccccc6-c6c5ccc5c6c6ccccc6n5-c5ccccc5)c5cccc6c5oc5ccccc56)cc4)ccc32)cc1 ; c1ccc(-c2ccc(N(c3ccccc3)c3cc(-c4ccccc4)cc(N(c4ccccc4)c4ccc5c(c4)C4(c6ccccc6-5)c5ccccc5-c5c4ccc4c5c5ccccc5n4-c4ccccc4)c3)cc2)cc1]

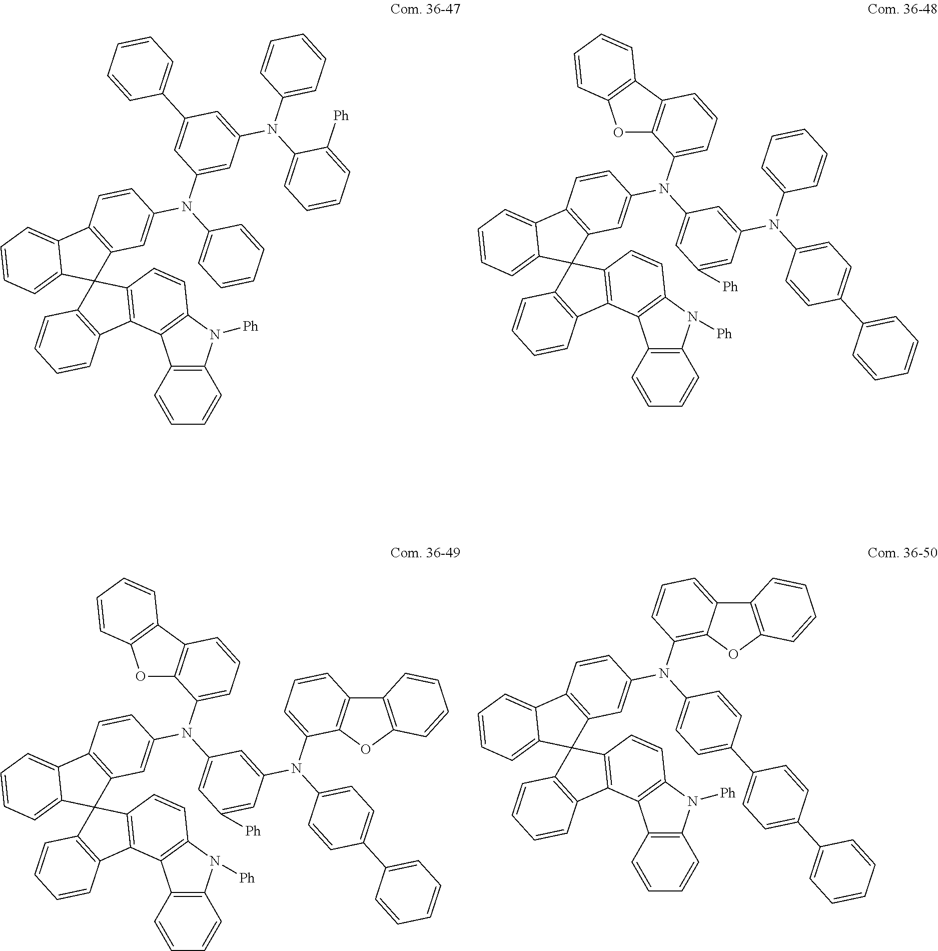

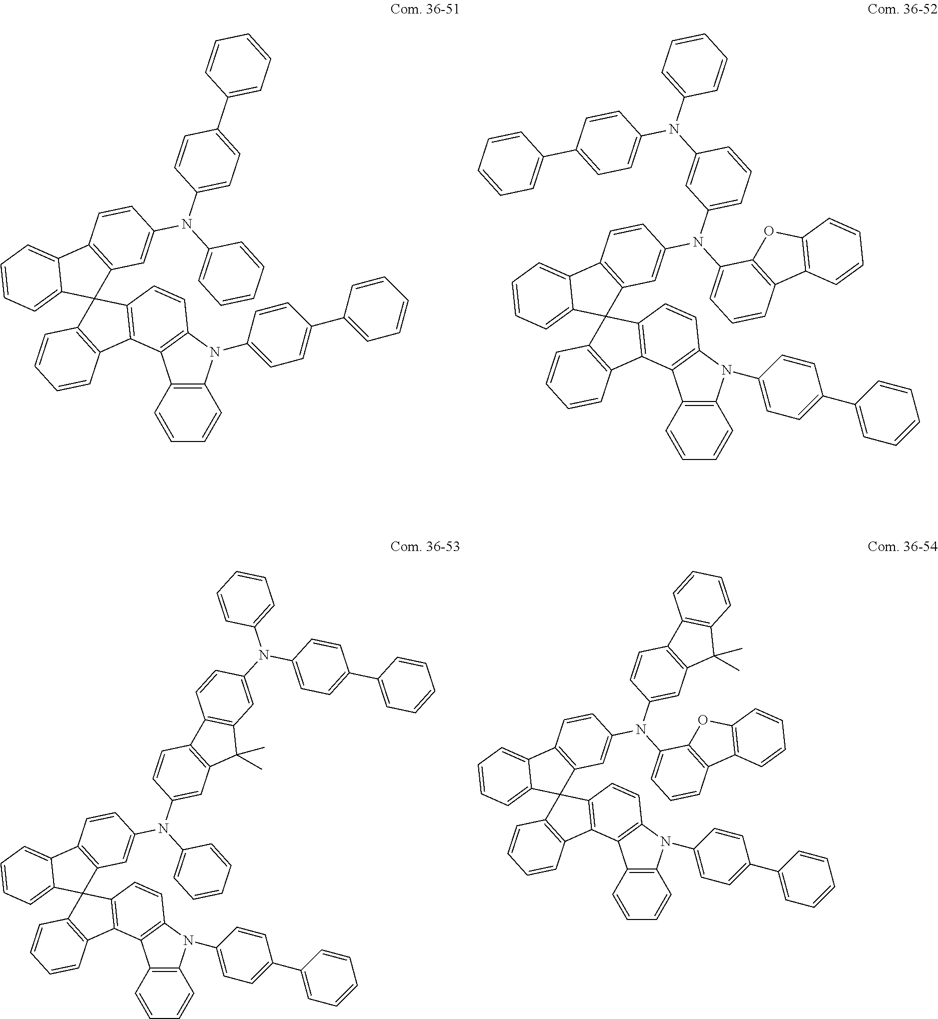

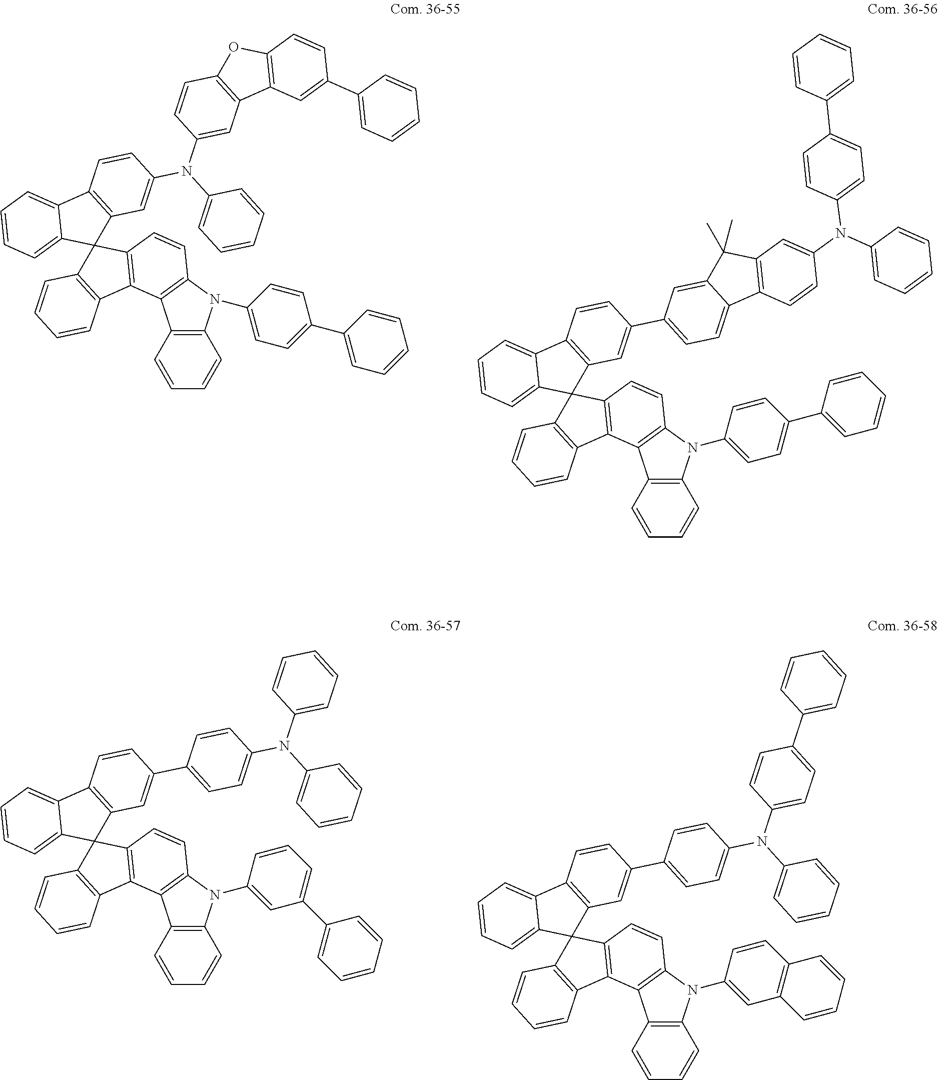 [SMILES: c1ccc(-c2ccc(-n3c4ccccc4c4c5c(ccc43)C3(c4ccccc4-c4ccc(N(c6ccccc6)c6ccc7oc8ccc(-c9ccccc9)cc8c7c6)cc43)c3ccccc3-5)cc2)cc1 ; CC1(C)c2cc(-c3ccc4c(c3)C3(c5ccccc5-4)c4ccccc4-c4c3ccc3c4c4ccccc4n3-c3ccc(-c4ccccc4)cc3)ccc2-c2ccc(N(c3ccccc3)c3ccc(-c4ccccc4)cc3)cc21 ; c1ccc(-c2cccc(-n3c4ccccc4c4c5c(ccc43)C3(c4ccccc4-c4ccc(-c6ccc(N(c7ccccc7)c7ccccc7)cc6)cc43)c3ccccc3-5)c2)cc1 ; c1ccc(-c2ccc(N(c3ccccc3)c3ccc(-c4ccc5c(c4)C4(c6ccccc6-5)c5ccccc5-c5c4ccc4c5c5ccccc5n4-c4ccc5ccccc5c4)cc3)cc2)cc1]

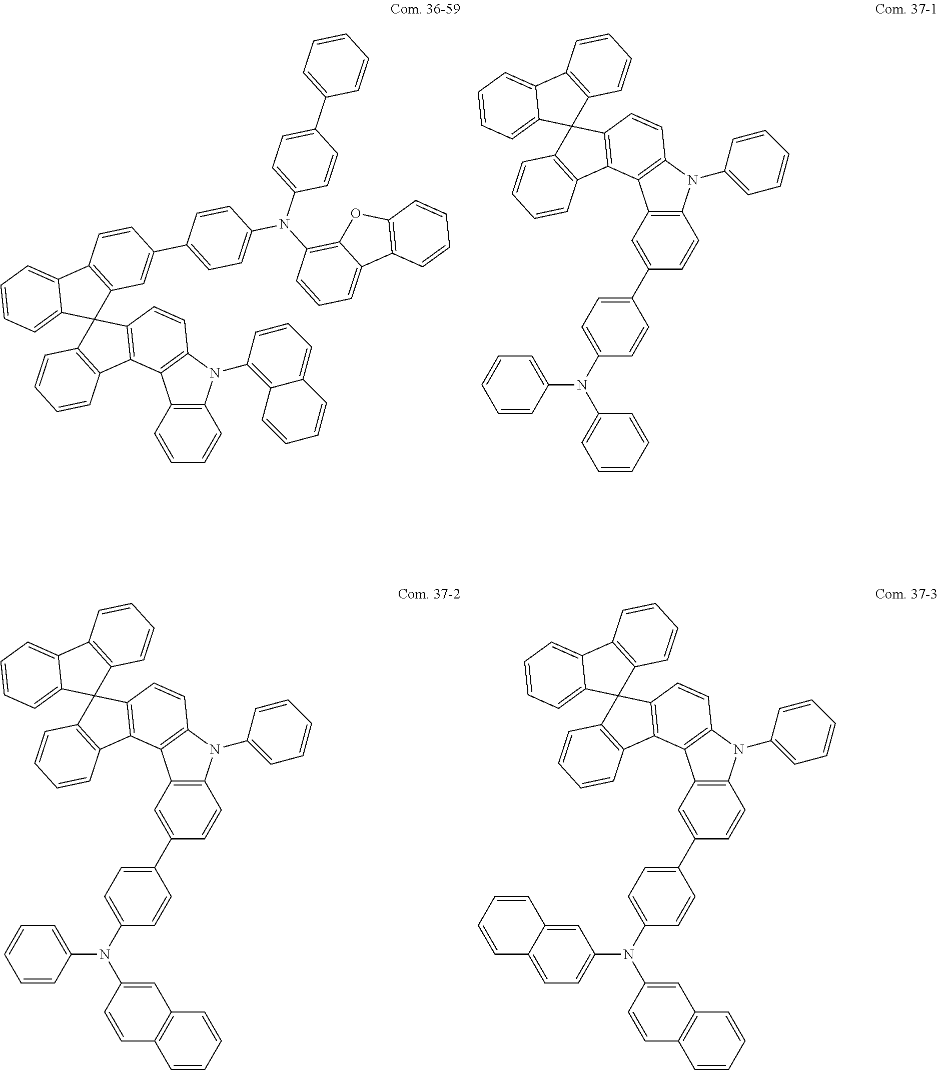 [SMILES: c1ccc(-c2ccc(N(c3ccc(-c4ccc5c(c4)C4(c6ccccc6-5)c5ccccc5-c5c4ccc4c5c5ccccc5n4-c4cccc5ccccc45)cc3)c3cccc4c3oc3ccccc34)cc2)cc1 ; c1ccc(N(c2ccccc2)c2ccc(-c3ccc4c(c3)c3c5c(ccc3n4-c3ccccc3)C3(c4ccccc4-c4ccccc43)c3ccccc3-5)cc2)cc1 ; c1ccc(N(c2ccc(-c3ccc4c(c3)c3c5c(ccc3n4-c3ccccc3)C3(c4ccccc4-c4ccccc43)c3ccccc3-5)cc2)c2ccc3ccccc3c2)cc1 ; c1ccc(-n2c3ccc(-c4ccc(N(c5ccc6ccccc6c5)c5ccc6ccccc6c5)cc4)cc3c3c4c(ccc32)C2(c3ccccc3-c3ccccc32)c2ccccc2-4)cc1]

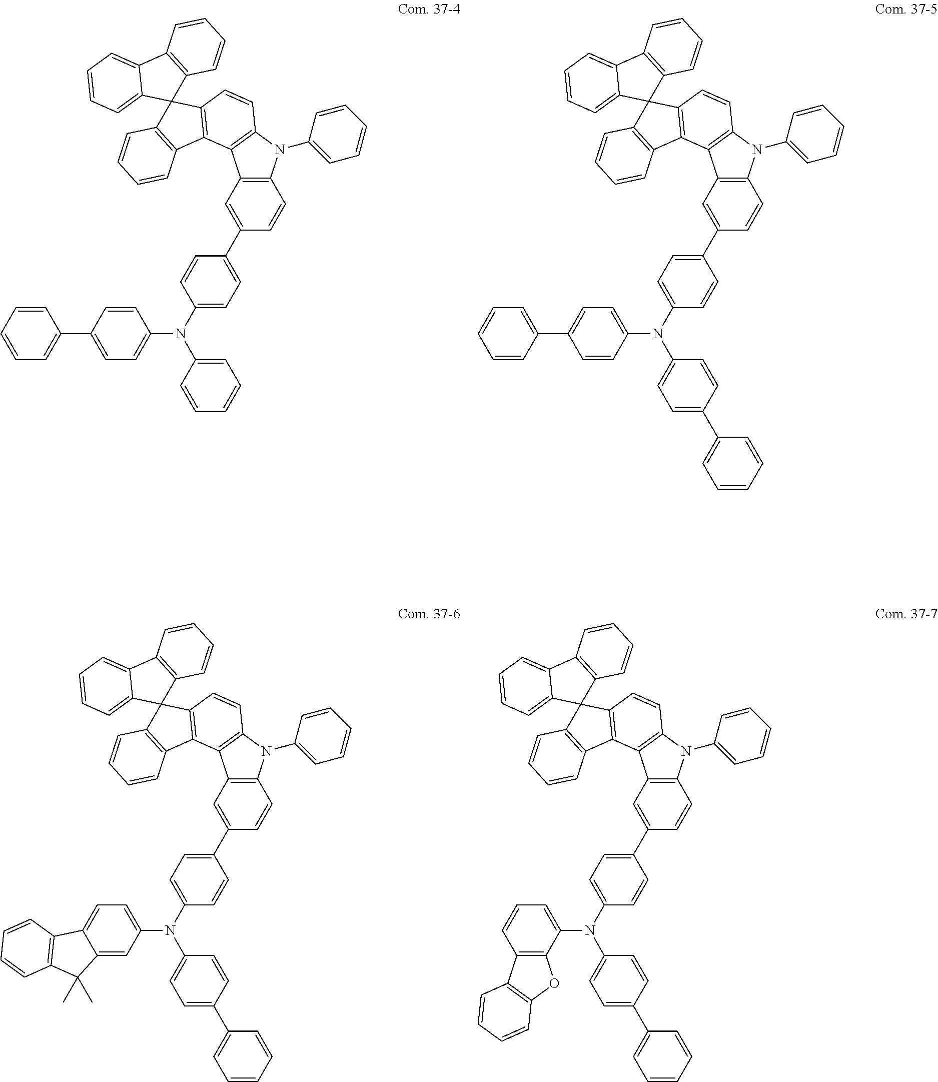 [SMILES: c1ccc(-c2ccc(N(c3ccccc3)c3ccc(-c4ccc5c(c4)c4c6c(ccc4n5-c4ccccc4)C4(c5ccccc5-c5ccccc54)c4ccccc4-6)cc3)cc2)cc1 ; c1ccc(-c2ccc(N(c3ccc(-c4ccccc4)cc3)c3ccc(-c4ccc5c(c4)c4c6c(ccc4n5-c4ccccc4)C4(c5ccccc5-c5ccccc54)c4ccccc4-6)cc3)cc2)cc1 ; CC1(C)c2ccccc2-c2ccc(N(c3ccc(-c4ccccc4)cc3)c3ccc(-c4ccc5c(c4)c4c6c(ccc4n5-c4ccccc4)C4(c5ccccc5-c5ccccc54)c4ccccc4-6)cc3)cc21 ; c1ccc(-c2ccc(N(c3ccc(-c4ccc5c(c4)c4c6c(ccc4n5-c4ccccc4)C4(c5ccccc5-c5ccccc54)c4ccccc4-6)cc3)c3cccc4c3oc3ccccc34)cc2)cc1]

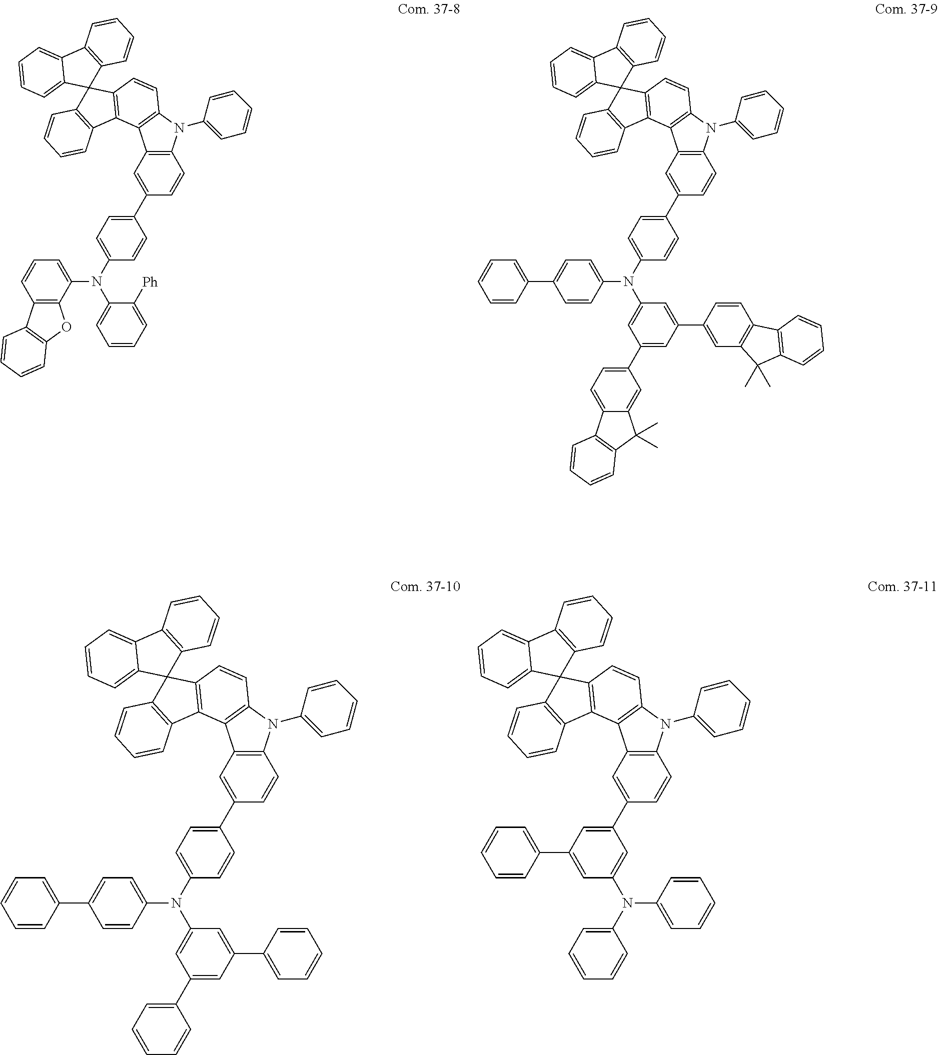

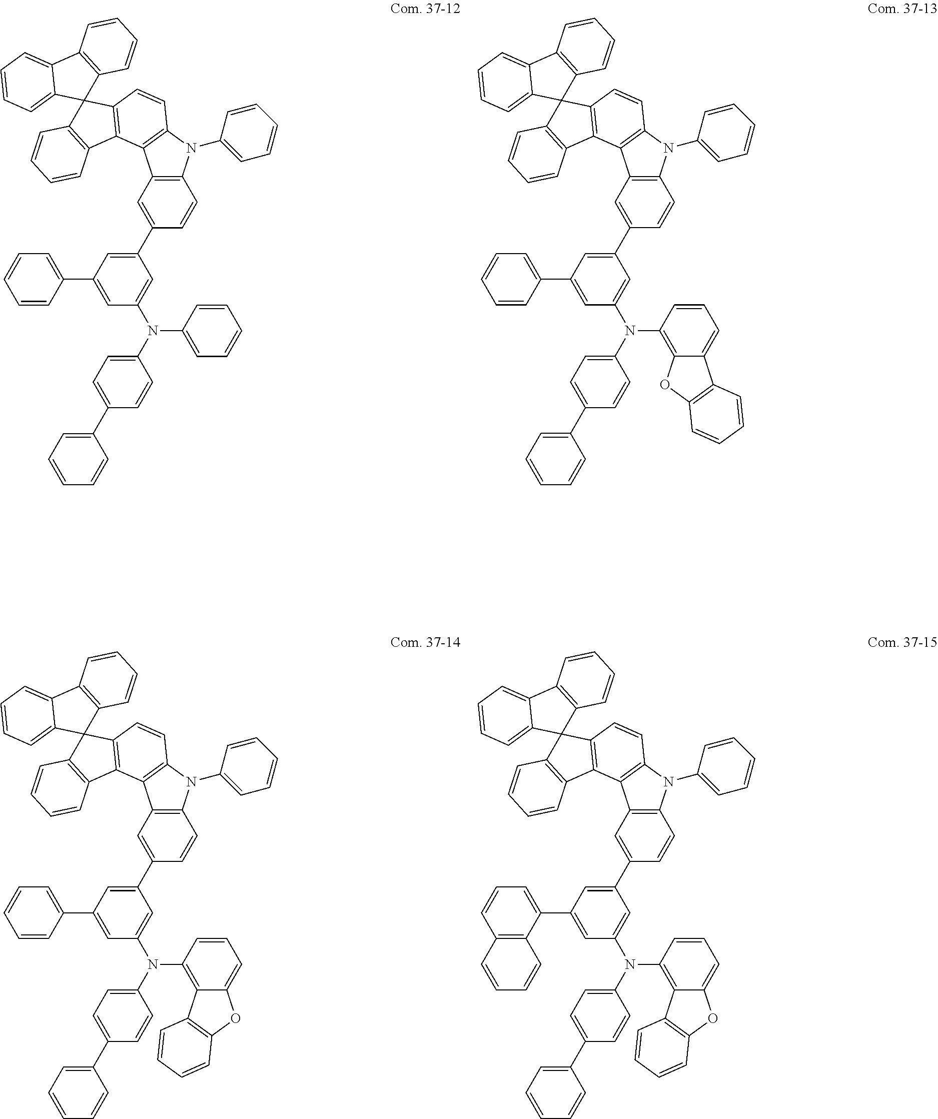

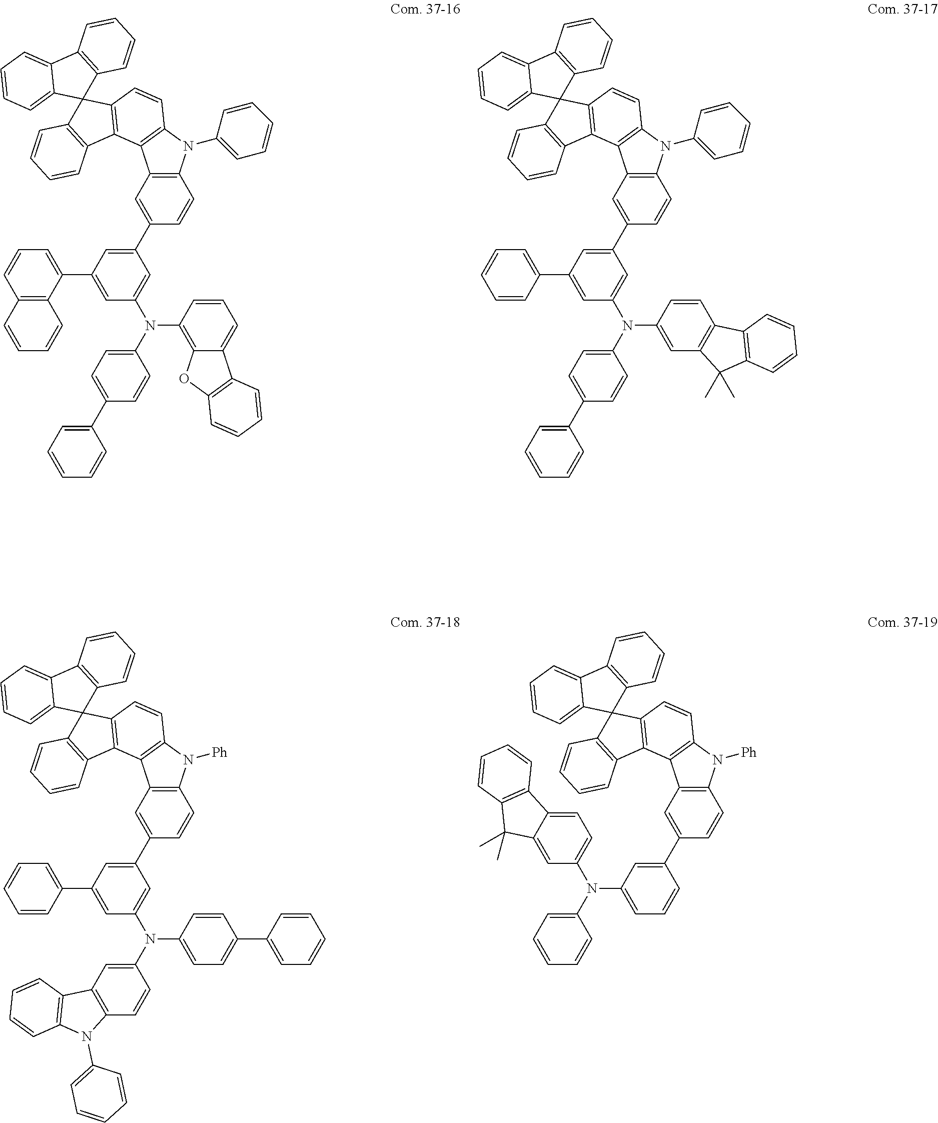

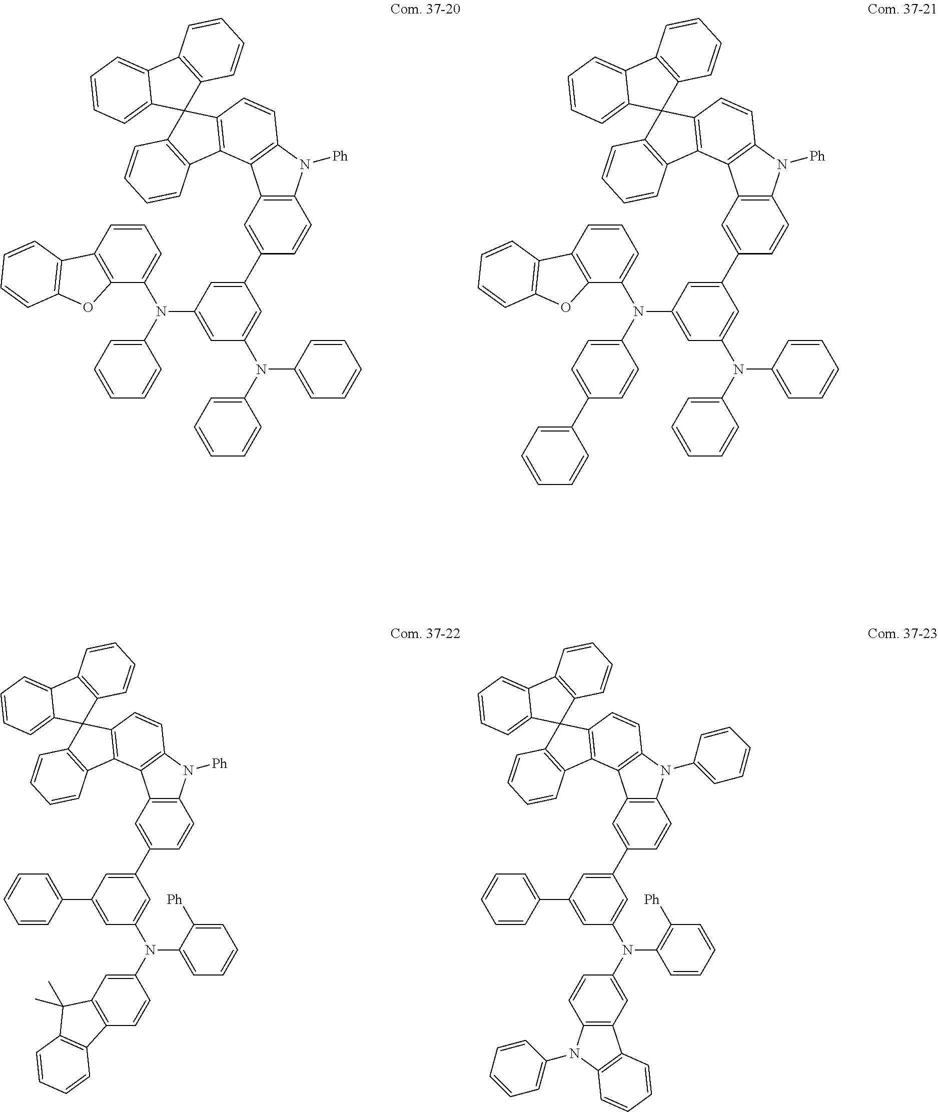

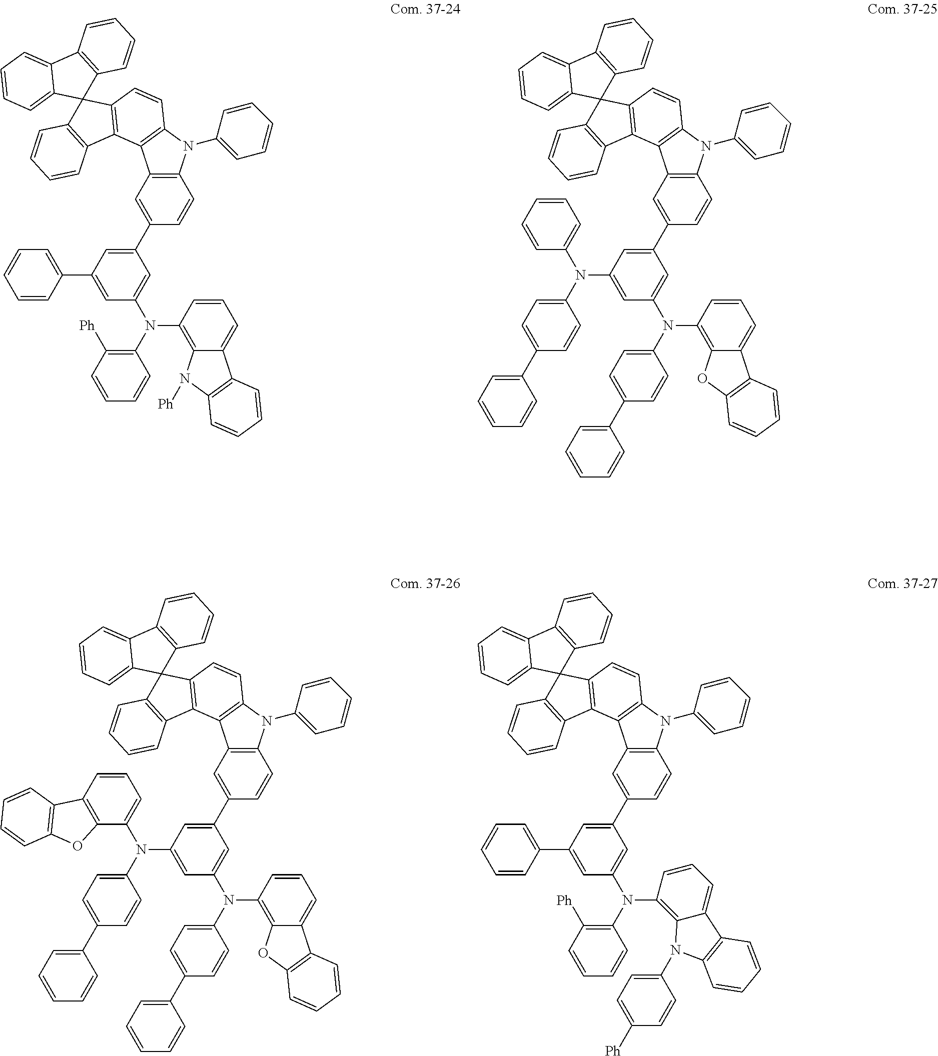

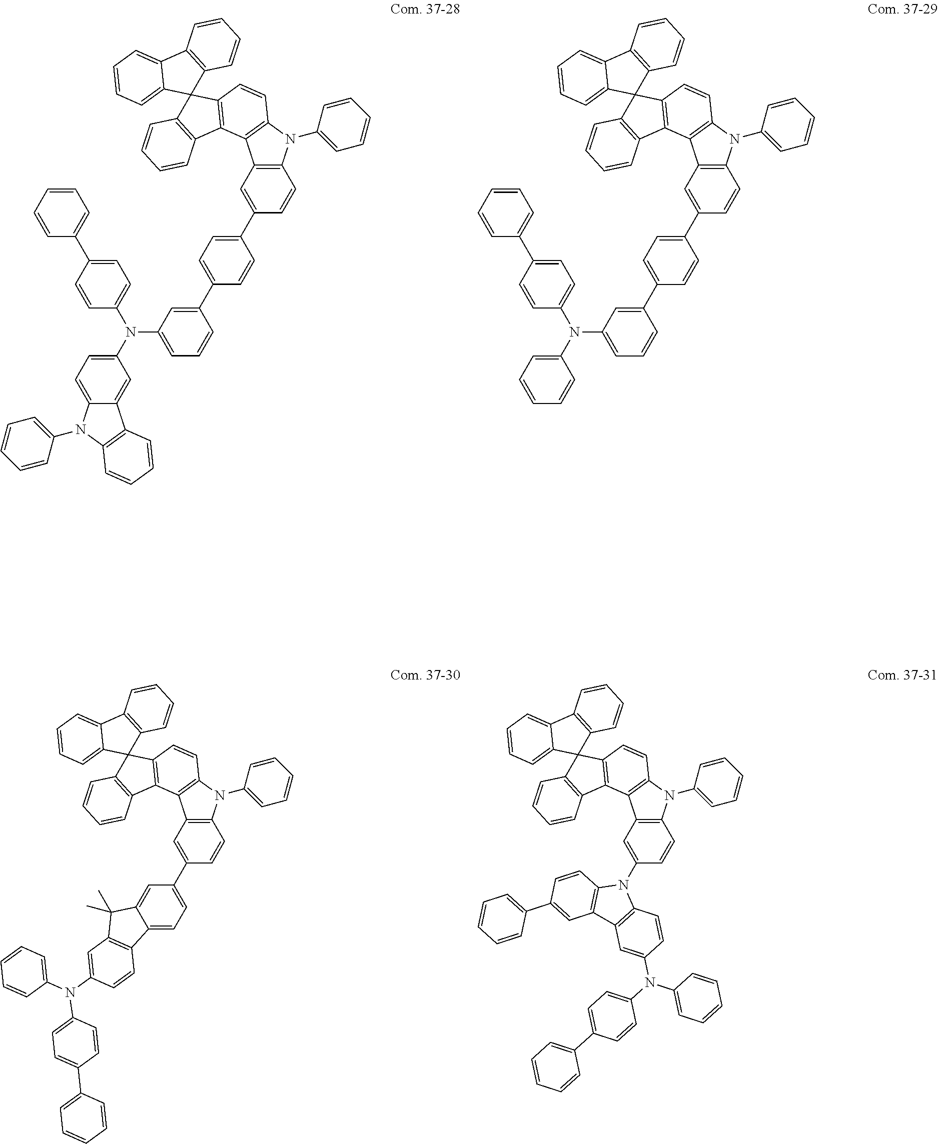

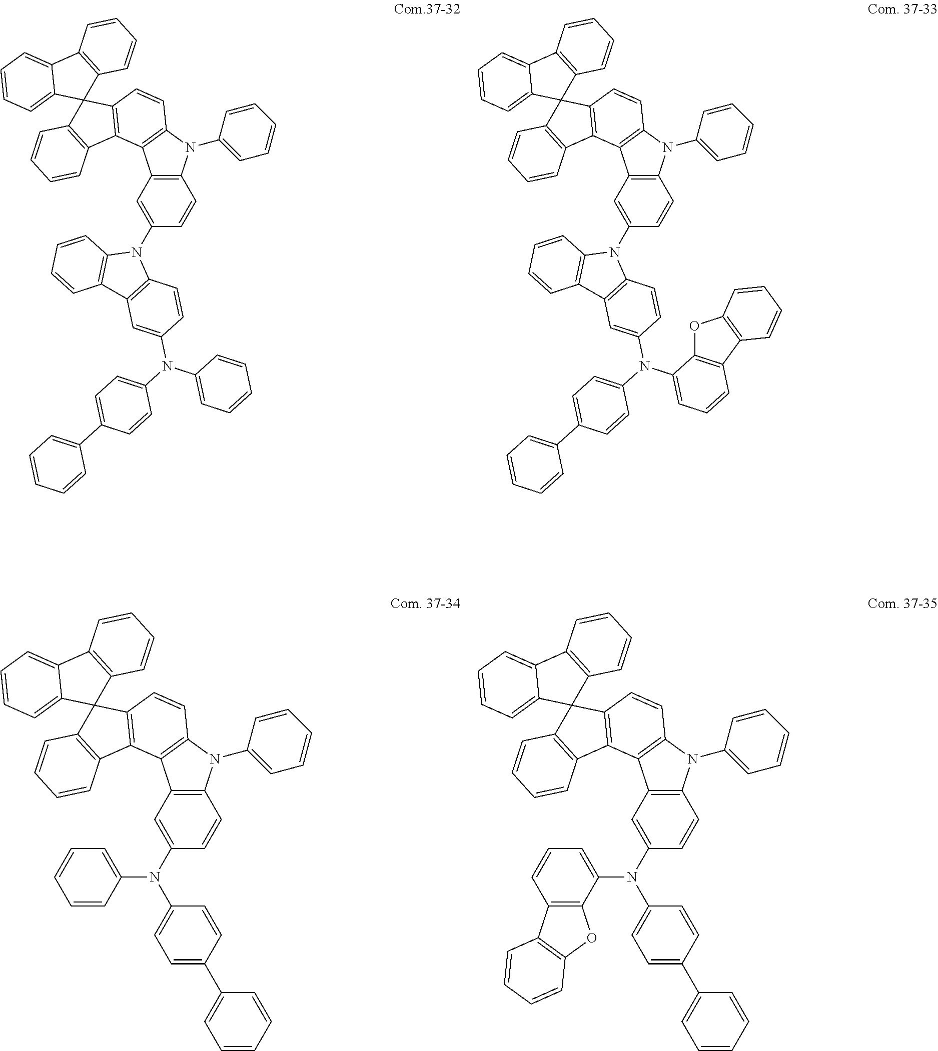 [SMILES: c1ccc(-c2ccc(N(c3ccccc3)c3ccc4c(c3)c3ccccc3n4-c3ccc4c(c3)c3c5c(ccc3n4-c3ccccc3)C3(c4ccccc4-c4ccccc43)c3ccccc3-5)cc2)cc1 ; c1ccc(-c2ccc(N(c3ccc4c(c3)c3ccccc3n4-c3ccc4c(c3)c3c5c(ccc3n4-c3ccccc3)C3(c4ccccc4-c4ccccc43)c3ccccc3-5)c3cccc4c3oc3ccccc34)cc2)cc1 ; c1ccc(-c2ccc(N(c3ccccc3)c3ccc4c(c3)c3c5c(ccc3n4-c3ccccc3)C3(c4ccccc4-c4ccccc43)c3ccccc3-5)cc2)cc1 ; c1ccc(-c2ccc(N(c3ccc4c(c3)c3c5c(ccc3n4-c3ccccc3)C3(c4ccccc4-c4ccccc43)c3ccccc3-5)c3cccc4c3oc3ccccc34)cc2)cc1]

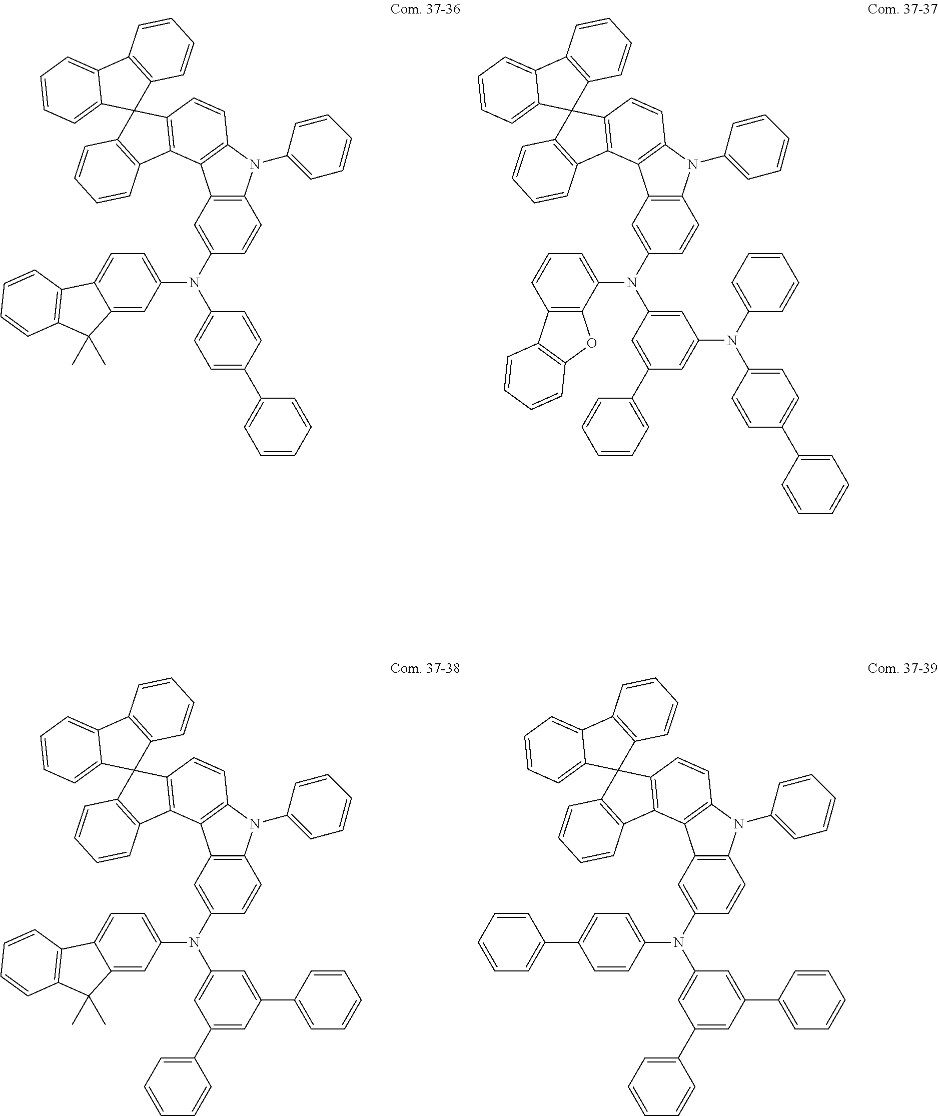

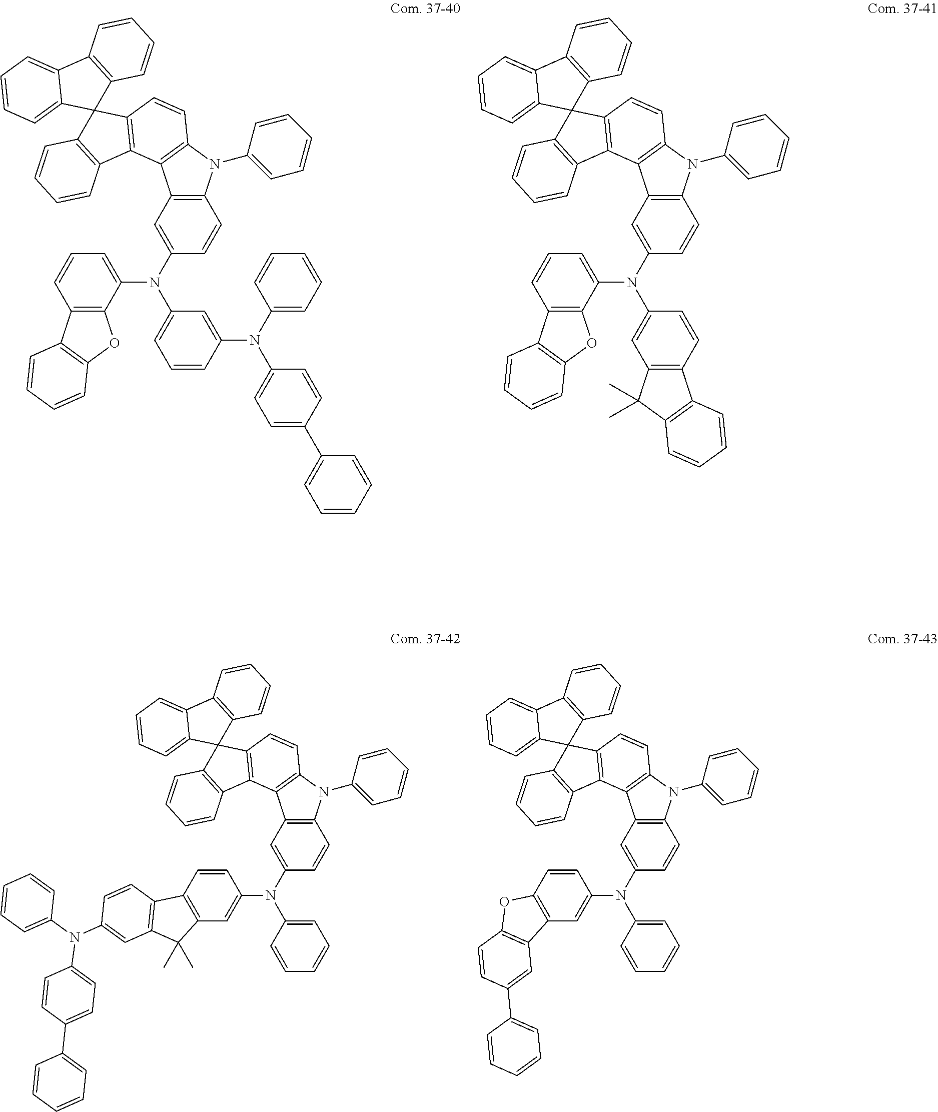 [SMILES: c1ccc(-c2ccc(N(c3ccccc3)c3cccc(N(c4ccc5c(c4)c4c6c(ccc4n5-c4ccccc4)C4(c5ccccc5-c5ccccc54)c4ccccc4-6)c4cccc5c4oc4ccccc45)c3)cc2)cc1 ; CC1(C)c2ccccc2-c2ccc(N(c3ccc4c(c3)c3c5c(ccc3n4-c3ccccc3)C3(c4ccccc4-c4ccccc43)c3ccccc3-5)c3cccc4c3oc3ccccc34)cc21 ; CC1(C)c2cc(N(c3ccccc3)c3ccc(-c4ccccc4)cc3)ccc2-c2ccc(N(c3ccccc3)c3ccc4c(c3)c3c5c(ccc3n4-c3ccccc3)C3(c4ccccc4-c4ccccc43)c3ccccc3-5)cc21 ; c1ccc(-c2ccc3oc4ccc(N(c5ccccc5)c5ccc6c(c5)c5c7c(ccc5n6-c5ccccc5)C5(c6ccccc6-c6ccccc65)c5ccccc5-7)cc4c3c2)cc1]

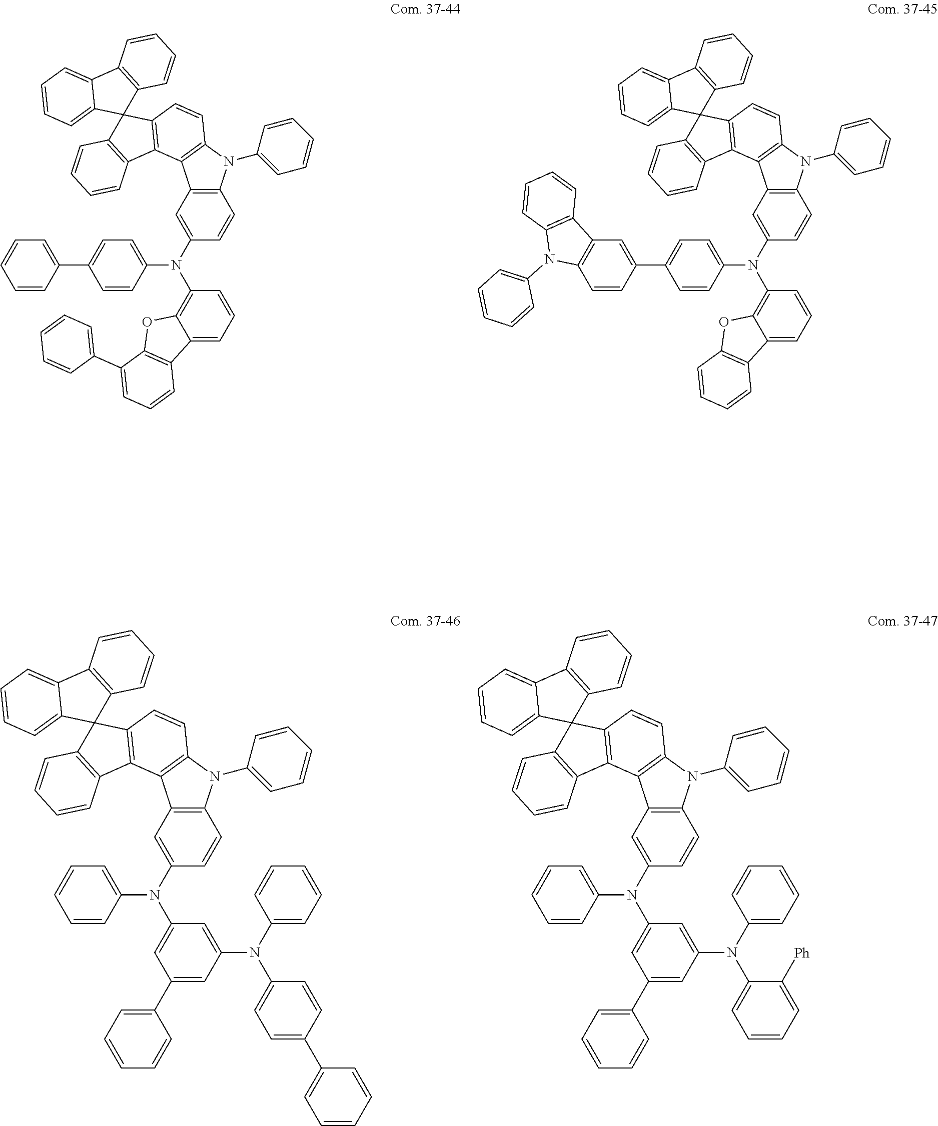

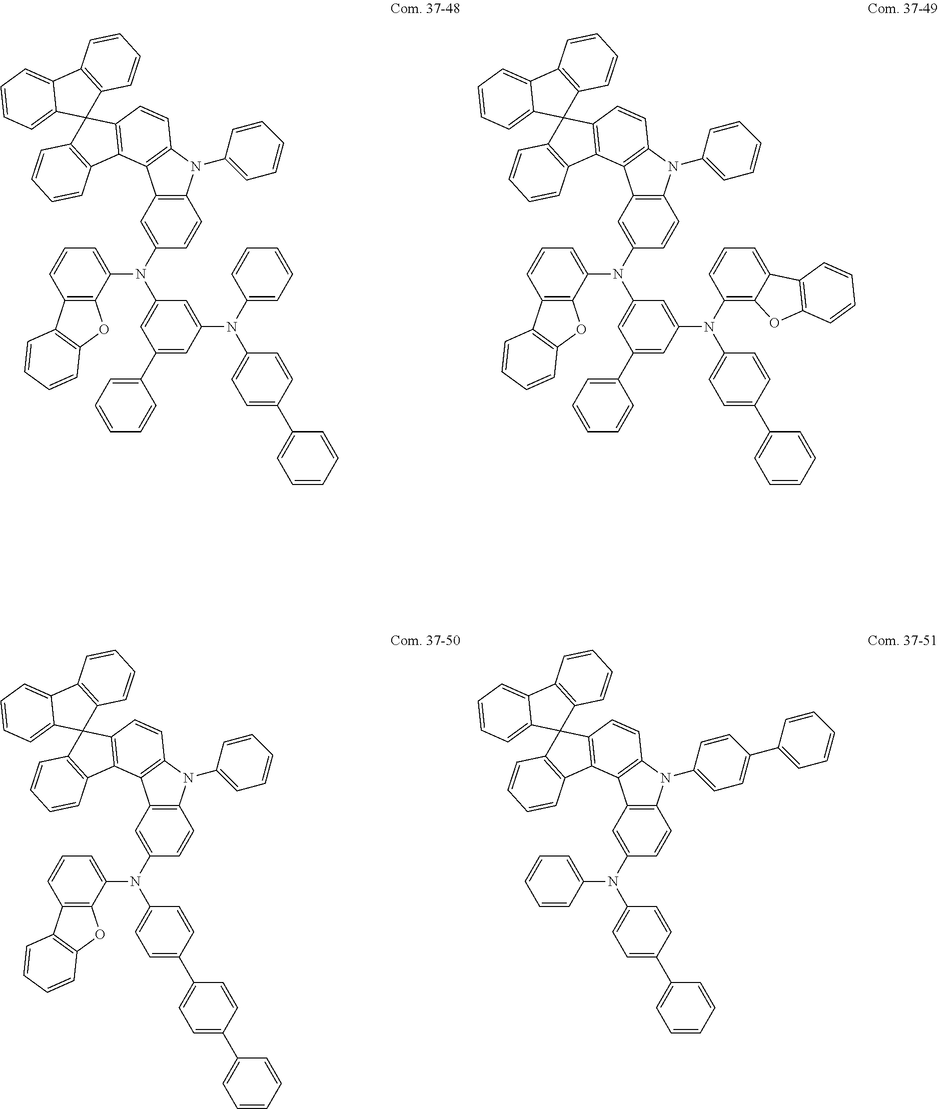

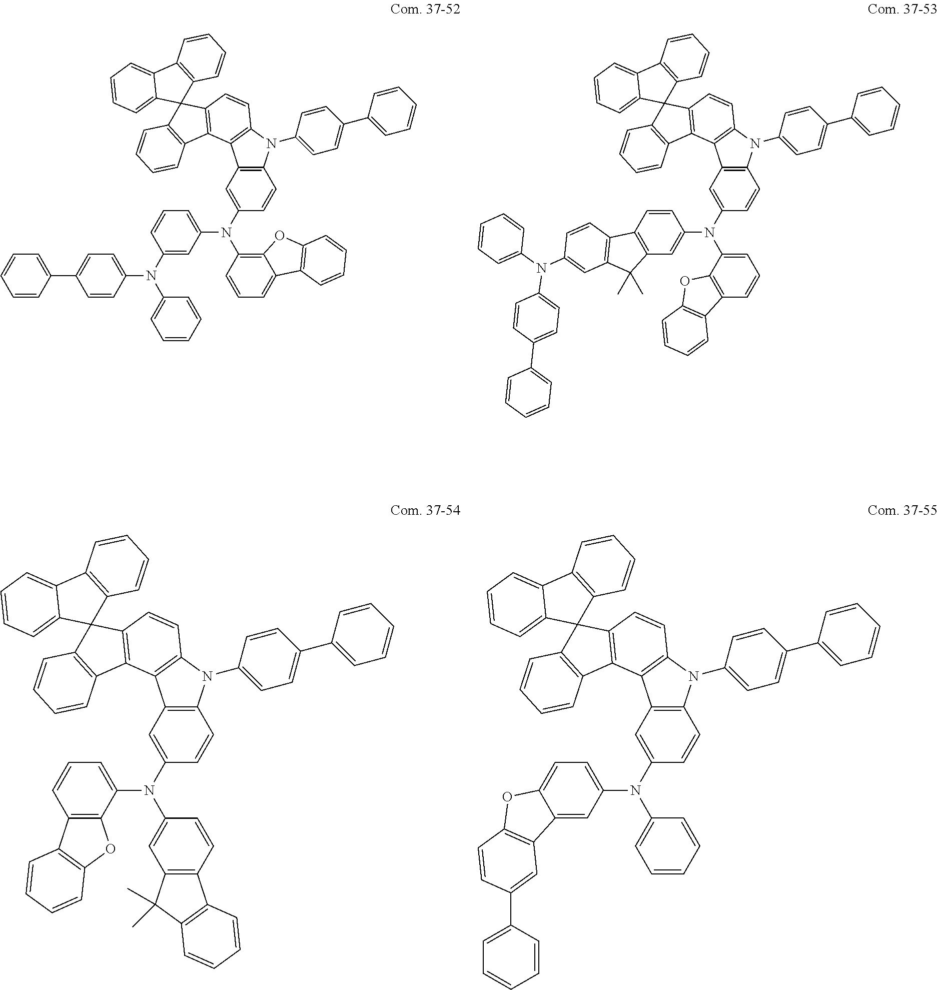

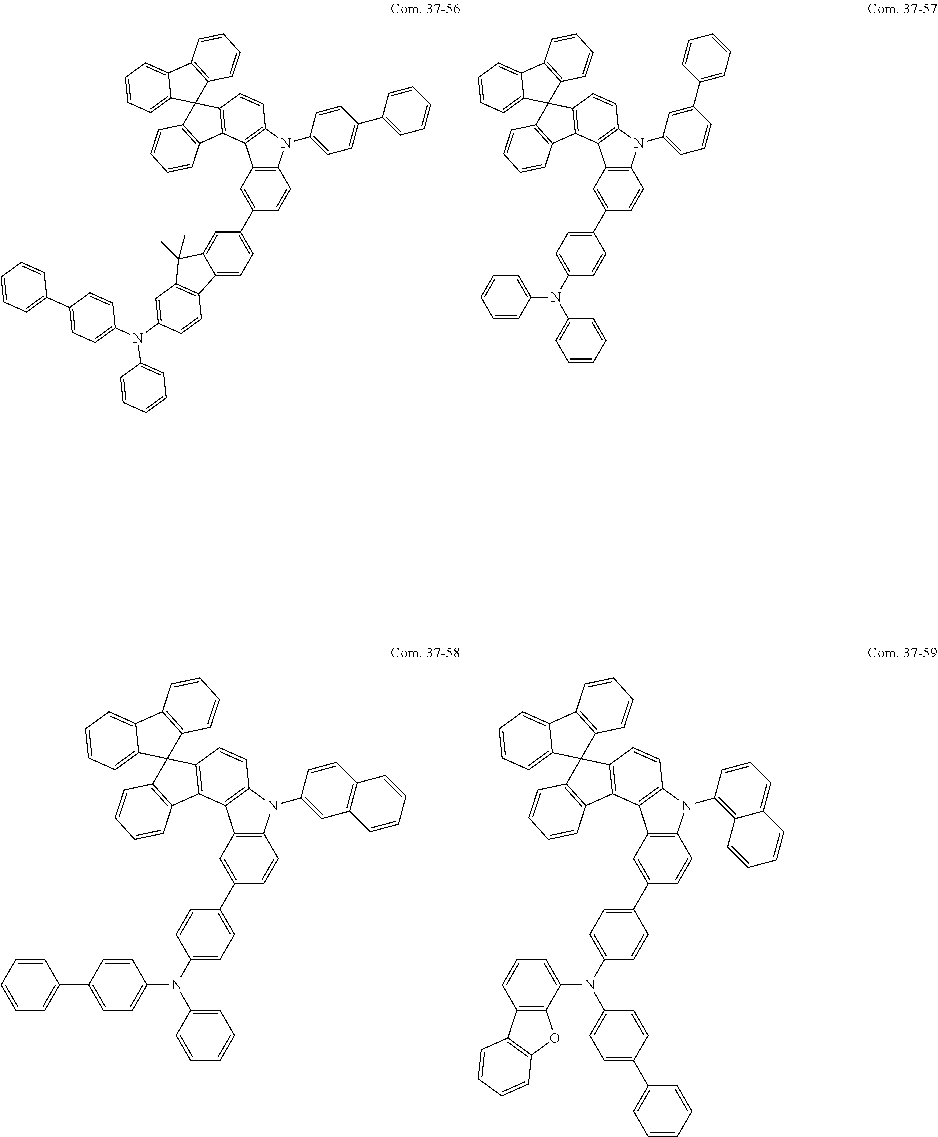 [SMILES: CC1(C)c2cc(-c3ccc4c(c3)c3c5c(ccc3n4-c3ccc(-c4ccccc4)cc3)C3(c4ccccc4-c4ccccc43)c3ccccc3-5)ccc2-c2ccc(N(c3ccccc3)c3ccc(-c4ccccc4)cc3)cc21 ; c1ccc(-c2cccc(-n3c4ccc(-c5ccc(N(c6ccccc6)c6ccccc6)cc5)cc4c4c5c(ccc43)C3(c4ccccc4-c4ccccc43)c3ccccc3-5)c2)cc1 ; c1ccc(-c2ccc(N(c3ccccc3)c3ccc(-c4ccc5c(c4)c4c6c(ccc4n5-c4ccc5ccccc5c4)C4(c5ccccc5-c5ccccc54)c4ccccc4-6)cc3)cc2)cc1 ; c1ccc(-c2ccc(N(c3ccc(-c4ccc5c(c4)c4c6c(ccc4n5-c4cccc5ccccc45)C4(c5ccccc5-c5ccccc54)c4ccccc4-6)cc3)c3cccc4c3oc3ccccc34)cc2)cc1]

8. An organic light-emitting device comprising: an anode; a cathode; and one or more organic material layers provided between the anode and the cathode,
wherein one or more layers of the organic material layers include the spirofluorene compound of claim 1.

9. The organic light-emitting device of claim 8, wherein the organic material layer is at least one layer selected from a hole injection layer, a hole transport layer, an emissive layer, an electron transport layer, and an electron injection layer.
